(19) United States
(12) Patent Application Publication
(10) Pub. No.: US 2024/0175047 A1
(43) Pub. Date: May 30, 2024

(54) Recombinant Vectors Comprising Polycistronic Expression Cassettes and Methods of Use Thereof

(71) Applicant: Alaunos Therapeutics, Inc., Houston, TX (US)

(72) Inventors: Drew Caldwell DENIGER, Houston, TX (US); Lenka Victoria HURTON, Houston, TX (US); Laurence James Neil COOPER, Houston, TX (US); Donghyun JOO, Houston, TX (US); Yaoyao SHI, Houston, TX (US); An LU, Houston, TX (US); Victor CARPIO, Houston, TX (US); Matthew COLLINSON-PAUTZ, Houston, TX (US)

(73) Assignee: Alaunos Therapeutics, Inc., Houston, TX (US)

(21) Appl. No.: 18/547,827

(22) PCT Filed: Feb. 17, 2022
(86) PCT No.: PCT/US2022/070690

§ 371 Date: Aug. 24, 2023

Related U.S. Application Data
(60) Provisional application No. 63/267,421, filed on Feb. 1, 2022.
(60) Provisional application No. 63/260,855, filed on Sep. 2, 2021.
(60) Provisional application No. 63/260,409, filed on Aug. 19, 2021.
(60) Provisional application No. 63/159,434, filed on Mar. 10, 2021.
(60) Provisional application No. 63/153,862, filed on Feb. 25, 2021.

(51) Int. Cl. C12N 15/85; A61K 39/00; C07K 14/54; C07K 14/725; C12N 5/0783

(52) U.S. Cl. CPC C12N 15/85; A61K 39/4611; C07K 14/5443; C07K 14/7051; C12N 5/0636; C07K 2319/03; C07K 2319/50; C12N 2800/90; C12N 2830/50

(57) ABSTRACT

Provided herein are vectors comprising a polycistronic expression casstte comprising a polynucleotide that encodes a TCR alpha chain, a polynucleotide that encodes a TCR beta chain, and a polynucleotide that encodes a cytokine, wherein the polynucleotides are separated by polynucleotide sequences that comprise 2A elements.

### 1. FIELD

The instant disclosure relates to polycistronic vectors comprising at least three cistrons and methods of using the same.

### 2. BACKGROUND

Co-expression of multiple genes in each cell of a population is critical for a wide variety of biomedical applications. A standard strategy for multigene expression is to incorporate the transgenes into multiple vectors and introduce each vector into the cell. However, the use of multiple vectors often produces a substantially heterogeneous population of engineered cells, wherein not all cells express each of the transgenes or do not express each of the transgenes to a similar degree. Such heterogeneity leads to several problems, particularly for therapeutic applications, including e.g., diminished persistence of the desired engineered cell phenotype in vivo, complex manufacturing and purification requirements, and lot-to-lot variability of the engineered cell product.

Given the problems associated with the use of multiple vectors to co-express multiple genes in single cells, there is an unmet need for single polycistronic vectors capable of not only expressing a plurality of transgenes in a single cell, but also of expressing some or all transgenes to a similar degree across a cell population, resulting in an engineered cell population optimized for therapeutic use.

### 3. SUMMARY

The instant disclosure provides recombinant vectors comprising a polycistronic expression cassette comprising a transcriptional regulatory element operably linked to a polycistronic polynucleotide.

Provided herein is a recombinant vector comprising a polycistronic expression cassette, wherein the polycistronic expression cassette comprises a transcriptional regulatory element operably linked to a polycistronic polynucleotide that comprises: a first polynucleotide sequence that encodes a T cell receptor (TCR) alpha chain comprising an alpha chain variable (Vα) region and an alpha chain constant (Cα) region; a second polynucleotide sequence that comprises a first 2A element; a third polynucleotide sequence that encodes a TCR beta chain comprising a beta chain variable (Vβ) region and a beta chain constant (Cβ) region; a fourth polynucleotide sequence that comprises a second 2A element; and a fifth polynucleotide sequence that encodes a fusion protein that comprises IL-15, or a functional fragment or functional variant thereof, and IL-15Rα, or a functional fragment or functional variant thereof.

In some embodiments, either or both of the first 2A element and the second 2A element, independently, is a P2A element, a T2A element, an F2A element, or an E2A element.

In some embodiments, the first 2A element is a P2A element.

In some embodiments, the P2A element comprises a polynucleotide sequence that encodes the amino acid sequence of SEQ ID NO: 18 or 20, or the amino acid sequence of SEQ ID NO: 18 or 20 comprising 1, 2, or 3 amino acid modifications.

In some embodiments, the P2A element comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 19 or 21.

In some embodiments, the second 2A element is a T2A element.

In some embodiments, the T2A element comprises a polynucleotide sequence that encodes the amino acid sequence of SEQ ID NO: 22 or 24, or the amino acid sequence of SEQ ID NO: 22 or 24 comprising 1, 2, or 3 amino acid modifications.

In some embodiments, the T2A element comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 23 or 25.

In some embodiments, either or both of the second polynucleotide sequence and the fourth polynucleotide sequence, independently, encode a furin recognition site.

In some embodiments, the furin recognition site comprises the amino acid sequence of SEQ ID NO: 2 or 4, or the amino acid sequence of SEQ ID NO: 2 or 4 comprising 1, 2, or 3 amino acid modifications.

In some embodiments, the furin recognition site is encoded by the polynucleotide sequence of SEQ ID NO: 3 or 5 or the polynucleotide sequence of SEQ ID NO: 3 or 5 comprising 1, 2, or 3 nucleotide modifications.

In some embodiments, the second polynucleotide sequence comprises a polynucleotide sequence that encodes the amino acid sequence of SEQ ID NO: 10, or the amino acid sequence of SEQ ID NO: 10 comprising 1, 2, or 3 amino acid modifications.

In some embodiments, the second polynucleotide sequence comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 11.

In some embodiments, the fourth polynucleotide sequence comprises a polynucleotide sequence that encodes the amino acid sequence of SEQ ID NO: 12, or the amino acid sequence of SEQ ID NO: 12 comprising 1, 2, or 3 amino acid modifications.

In some embodiments, the fourth polynucleotide sequence comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 13.

In some embodiments, the IL-15, or functional fragment or functional variant thereof, comprises an amino acid sequence at least 90%, 91%, 92%, 93%, 94%, 95%, 96%, 97%, 98%, 99%, or 100% identical to the amino acid sequence of SEQ ID NO: 76.

In some embodiments, the IL-15, or functional fragment or functional variant thereof, is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 77.

In some embodiments, the IL-15Rα, or functional fragment or functional variant thereof, comprises an amino acid sequence at least 90%, 91%, 92%, 93%, 94%, 95%, 96%, 97%, 98%, 99%, or 100% identical to the amino acid sequence of SEQ ID NO: 78.

In some embodiments, the IL-15Rα, or functional fragment or functional variant thereof, is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 79.

In some embodiments, the IL-15, or functional fragment or functional variant thereof, is operably linked to the IL-15Rα, or functional fragment or functional variant thereof, via a peptide linker.

In some embodiments, the peptide linker comprises the amino acid sequence of SEQ ID NO: 81, or the amino acid sequence of SEQ ID NO: 81 comprising 1, 2, or 3 amino acid modifications.

In some embodiments, the peptide linker is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 82.

In some embodiments, the fusion protein is membrane bound.

In some embodiments, the fusion protein comprises an amino acid sequence at least 90%, 91%, 92%, 93%, 94%, 95%, 96%, 97%, 98%, 99%, or 100% identical to the amino acid sequence of SEQ ID NO: 70 or 73.

In some embodiments, the fusion protein is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 71 or 74.

In some embodiments, the Cα region comprises an amino acid sequence at least 90%, 91%, 92%, 93%, 94%, 95%, 96%, 97%, 98%, 99%, or 100% identical to the amino acid sequence of SEQ ID NO: 40-49.

In some embodiments, the Cα region is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 55, 57, or 58.

In some embodiments, the Cβ region comprises an amino acid sequence at least 90%, 91%, 92%, 93%, 94%, 95%, 96%, 97%, 98%, 99%, or 100% identical to the amino acid sequence of SEQ ID NO: 50-54 or 60.

In some embodiments, the Cβ region is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 56 or 59.

In some embodiments, the polycistronic polynucleotide comprises, in order from 5′ to 3′: the first polynucleotide sequence, the second polynucleotide sequence, the third polynucleotide sequence, the fourth polynucleotide sequence, and the fifth polynucleotide sequence.

In some embodiments, the first polynucleotide sequence and the second polynucleotide sequence together comprise a first combination polynucleotide sequence that encodes the amino acid sequence of SEQ ID NO: 160; the third polynucleotide sequence and the fourth polynucleotide sequence together comprise a second combination polynucleotide sequence that encodes the amino acid sequence of SEQ ID NO: 168; or the third polynucleotide sequence, the fourth polynucleotide sequence, and the fifth polynucleotide sequence together comprise a third combination polynucleotide sequence that encodes the amino acid sequence of SEQ ID NO: 161.

In some embodiments, the first combination polynucleotide sequence comprises the polynucleotide sequence of SEQ ID NO: 230; the second combination polynucleotide sequence comprises the polynucleotide sequence of SEQ ID NO: 231; or the third combination polynucleotide sequence comprises the polynucleotide sequence of SEQ ID NO: 232.

In some embodiments, the first polynucleotide sequence and the second polynucleotide sequence together comprise a first combination polynucleotide sequence that encodes the amino acid sequence of SEQ ID NO: 160; and the third polynucleotide sequence, the fourth polynucleotide sequence, and the fifth polynucleotide sequence together comprise a third combination polynucleotide sequence that encodes the amino acid sequence of SEQ ID NO: 161.

In some embodiments, the first polynucleotide sequence and the second polynucleotide sequence together comprise a first combination polynucleotide sequence that encodes the amino acid sequence of SEQ ID NO: 180 or 210; and the third polynucleotide sequence, the fourth polynucleotide sequence, and the fifth polynucleotide sequence together comprise a third combination polynucleotide sequence that encodes the amino acid sequence of SEQ ID NO: 181.

In some embodiments, the first combination polynucleotide sequence comprises the polynucleotide sequence of SEQ ID NO: 230; and the third combination polynucleotide sequence comprises the polynucleotide sequence of SEQ ID NO: 232.

In some embodiments, the first combination polynucleotide sequence comprises the polynucleotide sequence of SEQ ID NO: 250 or 270; and the third combination polynucleotide sequence comprises the polynucleotide sequence of SEQ ID NO: 252.

In some embodiments, the polycistronic polynucleotide comprises, in order from 5′ to 3′: the first polynucleotide sequence, the fourth polynucleotide sequence, the third polynucleotide sequence, the second polynucleotide sequence, and the fifth polynucleotide sequence.

In some embodiments, the first polynucleotide sequence and the fourth polynucleotide sequence together comprise a fourth combination polynucleotide sequence that encodes the amino acid sequence of SEQ ID NO: 162; the third polynucleotide sequence and the second polynucleotide sequence together comprise a fifth combination polynucleotide sequence that encodes the amino acid sequence of SEQ ID NO: 166; or the third polynucleotide sequence, the second polynucleotide sequence, and the fifth polynucleotide sequence together comprise a sixth combination polynucleotide sequence that encodes the amino acid sequence of SEQ ID NO: 163.

In some embodiments, the fourth combination polynucleotide sequence comprises the polynucleotide sequence of SEQ ID NO: 233; the fifth combination polynucleotide sequence comprises the polynucleotide sequence of SEQ ID NO: 234; or the sixth combination polynucleotide sequence comprises the polynucleotide sequence of SEQ ID NO: 235.

In some embodiments, the first polynucleotide sequence and the fourth polynucleotide sequence together comprise a fourth combination polynucleotide sequence that encodes the amino acid sequence of SEQ ID NO: 162; and the third polynucleotide sequence, the second polynucleotide sequence, and the fifth polynucleotide sequence together comprise a sixth combination polynucleotide sequence that encodes the amino acid sequence of SEQ ID NO: 163.

In some embodiments, the first polynucleotide sequence and the fourth polynucleotide sequence together comprise a fourth combination polynucleotide sequence that encodes the amino acid sequence of SEQ ID NO: 182 or 212; and the third polynucleotide sequence, the second polynucleotide sequence, and the fifth polynucleotide sequence together comprise a sixth combination polynucleotide sequence that encodes the amino acid sequence of SEQ ID NO: 183.

In some embodiments, the fourth combination polynucleotide sequence comprises the polynucleotide sequence of SEQ ID NO: 233; and the sixth combination polynucleotide sequence comprises the polynucleotide sequence of SEQ ID NO: 235.

In some embodiments, the fourth combination polynucleotide sequence comprises the polynucleotide sequence of SEQ ID NO: 253 or 273; and the sixth combination polynucleotide sequence comprises the polynucleotide sequence of SEQ ID NO: 255.

In some embodiments, the polycistronic polynucleotide comprises, in order from 5′ to 3′: the first polynucleotide sequence, the second polynucleotide sequence, the fifth polynucleotide sequence, the fourth polynucleotide sequence, and the third polynucleotide sequence.

In some embodiments, the first polynucleotide sequence and the second polynucleotide sequence together comprise a first combination polynucleotide sequence that encodes the amino acid sequence of SEQ ID NO: 160; the first polynucleotide sequence, the second polynucleotide sequence, and the fifth polynucleotide sequence together comprise a seventh combination polynucleotide sequence that encodes the amino acid sequence of SEQ ID NO: 169; the fifth polynucleotide sequence and the fourth polynucleotide sequence together comprise an eighth combination polynucleotide sequence that encodes the amino acid sequence of SEQ ID NO: 173; or the first polynucleotide sequence, the second polynucleotide sequence, the fifth polynucleotide sequence, and the fourth polynucleotide sequence together comprise a ninth combination polynucleotide sequence that encodes the amino acid sequence of SEQ ID NO: 164.

In some embodiments, the first combination polynucleotide sequence comprises the polynucleotide sequence of SEQ ID NO: 230; the seventh combination polynucleotide sequence comprises the polynucleotide sequence of SEQ ID NO: 236; the eighth combination polynucleotide sequence comprises the polynucleotide sequence of SEQ ID NO: 237; or the ninth combination polynucleotide sequence comprises the polynucleotide sequence of SEQ ID NO: 238.

In some embodiments, the first polynucleotide sequence, the second polynucleotide sequence, the fifth polynucleotide sequence, and the fourth polynucleotide sequence together comprise a ninth combination polynucleotide sequence that encodes the amino acid sequence of SEQ ID NO: 164; and the third polynucleotide sequence encodes the amino acid sequence of SEQ ID NO: 50.

In some embodiments, the first polynucleotide sequence, the second polynucleotide sequence, the fifth polynucleotide sequence, and the fourth polynucleotide sequence together comprise a ninth combination polynucleotide sequence that encodes the amino acid sequence of SEQ ID NO: 184 or 214; and the third polynucleotide sequence encodes the amino acid sequence of SEQ ID NO: 51.

In some embodiments, the ninth combination polynucleotide sequence comprises the polynucleotide sequence of SEQ ID NO: 238; and the third polynucleotide sequence comprises the polynucleotide sequence of SEQ ID NO: 59.

In some embodiments, the ninth combination polynucleotide sequence comprises the polynucleotide sequence of SEQ ID NO: 258 or 278; and the third polynucleotide sequence comprises the polynucleotide sequence of SEQ ID NO: 56.

In some embodiments, the polycistronic polynucleotide comprises, in order from 5′ to 3′: the first polynucleotide sequence, the fourth polynucleotide sequence, the fifth polynucleotide sequence, the second polynucleotide sequence, and the third polynucleotide sequence.

In some embodiments, the first polynucleotide sequence and the fourth polynucleotide sequence together comprise a fourth combination polynucleotide sequence that encodes the amino acid sequence of SEQ ID NO: 162; the first polynucleotide sequence, the fourth polynucleotide sequence, and the fifth polynucleotide sequence together comprise a tenth combination polynucleotide sequence that encodes the amino acid sequence of SEQ ID NO: 167; the fifth polynucleotide sequence and the second polynucleotide sequence together comprise an eleventh combination polynucleotide sequence that encodes the amino acid sequence of SEQ ID NO: 172; or the first polynucleotide sequence, the fourth polynucleotide sequence, the fifth polynucleotide sequence, and the second polynucleotide sequence together comprise a twelfth combination polynucleotide sequence that encodes the amino acid sequence of SEQ ID NO: 165.

In some embodiments, the fourth combination polynucleotide sequence comprises the polynucleotide sequence of SEQ ID NO: 233; the tenth combination polynucleotide sequence comprises the polynucleotide sequence of SEQ ID NO: 239; the eleventh combination polynucleotide sequence comprises the polynucleotide sequence of SEQ ID NO: 240; or the twelfth combination polynucleotide sequence comprises the polynucleotide sequence of SEQ ID NO: 241.

In some embodiments, the first polynucleotide sequence, the fourth polynucleotide sequence, the fifth polynucleotide sequence, and the second polynucleotide sequence together comprise a twelfth combination polynucleotide sequence that encodes the amino acid sequence of SEQ ID NO: 165; and the third polynucleotide sequence encodes the amino acid sequence of SEQ ID NO: 50.

In some embodiments, the first polynucleotide sequence, the fourth polynucleotide sequence, the fifth polynucleotide sequence, and the second polynucleotide sequence together comprise a twelfth combination polynucleotide sequence that encodes the amino acid sequence of SEQ ID NO: 185 or 215; and the third polynucleotide sequence encodes the amino acid sequence of SEQ ID NO: 51.

In some embodiments, the twelfth combination polynucleotide sequence comprises the polynucleotide sequence of SEQ ID NO: 241; and the third polynucleotide sequence comprises the polynucleotide sequence of SEQ ID NO: 59.

In some embodiments, the twelfth combination polynucleotide sequence comprises the polynucleotide sequence of SEQ ID NO: 261 or 281; and the third polynucleotide sequence comprises the polynucleotide sequence of SEQ ID NO: 56.

In some embodiments, the polycistronic polynucleotide comprises, in order from 5′ to 3′: the third polynucleotide sequence, the second polynucleotide sequence, the first polynucleotide sequence, the fourth polynucleotide sequence, and the fifth polynucleotide sequence.

In some embodiments, the first polynucleotide sequence and the fourth polynucleotide sequence together comprise a fourth combination polynucleotide sequence that encodes the amino acid sequence of SEQ ID NO: 162; the third polynucleotide sequence and the second polynucleotide sequence together comprise a fifth combination polynucleotide sequence that encodes the amino acid sequence of SEQ ID NO: 166; or the first polynucleotide sequence, the fourth polynucleotide sequence, and the fifth polynucleotide sequence together comprise a tenth combination polynucleotide sequence that encodes the amino acid sequence of SEQ ID NO: 167.

In some embodiments, the fourth combination polynucleotide sequence comprises the polynucleotide sequence of SEQ ID NO: 233; the fifth combination polynucleotide sequence comprises the polynucleotide sequence of SEQ ID NO: 234; or the tenth combination polynucleotide sequence comprises the polynucleotide sequence of SEQ ID NO: 239.

In some embodiments, the third polynucleotide sequence and the second polynucleotide sequence together comprise a fifth combination polynucleotide sequence that encodes the amino acid sequence of SEQ ID NO: 166; and the first polynucleotide sequence, the fourth polynucleotide sequence, and the fifth polynucleotide sequence together comprise a tenth combination polynucleotide sequence that encodes the amino acid sequence of SEQ ID NO: 167.

In some embodiments, the third polynucleotide sequence and the second polynucleotide sequence together comprise a fifth combination polynucleotide sequence that encodes the amino acid sequence of SEQ ID NO: 186; and the first polynucleotide sequence, the fourth polynucleotide sequence, and the fifth polynucleotide sequence together comprise a tenth combination polynucleotide sequence that encodes the amino acid sequence of SEQ ID NO: 187 or 217.

In some embodiments, the fifth combination polynucleotide sequence comprises the polynucleotide sequence of SEQ ID NO: 234; and the tenth combination polynucleotide sequence comprises the polynucleotide sequence of SEQ ID NO: 239.

In some embodiments, the fifth combination polynucleotide sequence comprises the polynucleotide sequence of SEQ ID NO: 254; and the tenth combination polynucleotide sequence comprises the polynucleotide sequence of SEQ ID NO: 259 or 279.

In some embodiments, the polycistronic polynucleotide comprises, in order from 5′ to 3′: the third polynucleotide sequence, the fourth polynucleotide sequence, the first polynucleotide sequence, the second polynucleotide sequence, and the fifth polynucleotide sequence.

In some embodiments, the first polynucleotide sequence and the second polynucleotide sequence together comprise a first combination polynucleotide sequence that encodes the amino acid sequence of SEQ ID NO: 160; the third polynucleotide sequence, and the fourth polynucleotide sequence together comprise a second combination polynucleotide sequence that encodes the amino acid sequence of SEQ ID NO: 168; or the first polynucleotide sequence, the second polynucleotide sequence, and the fifth polynucleotide sequence together comprise a seventh combination polynucleotide sequence that encodes the amino acid sequence of SEQ ID NO: 169.

In some embodiments, the first combination polynucleotide sequence comprises the polynucleotide sequence of SEQ ID NO: 230; the second combination polynucleotide sequence comprises the polynucleotide sequence of SEQ ID NO: 231; or the seventh combination polynucleotide sequence comprises the polynucleotide sequence of SEQ ID NO: 236.

In some embodiments, the third polynucleotide sequence, and the fourth polynucleotide sequence together comprise a second combination polynucleotide sequence that encodes the amino acid sequence of SEQ ID NO: 168; and the first polynucleotide sequence, the second polynucleotide sequence, and the fifth polynucleotide sequence together comprise a seventh combination polynucleotide sequence that encodes the amino acid sequence of SEQ ID NO: 169.

In some embodiments, the third polynucleotide sequence, and the fourth polynucleotide sequence together comprise a second combination polynucleotide sequence that encodes the amino acid sequence of SEQ ID NO: 188; and the first polynucleotide sequence, the second polynucleotide sequence, and the fifth polynucleotide sequence together comprise a seventh combination polynucleotide sequence that encodes the amino acid sequence of SEQ ID NO: 189 or 219.

In some embodiments, the second combination polynucleotide sequence comprises the polynucleotide sequence of SEQ ID NO: 231; and the seventh combination polynucleotide sequence comprises the polynucleotide sequence of SEQ ID NO: 236.

In some embodiments, the second combination polynucleotide sequence comprises the polynucleotide sequence of SEQ ID NO: 251; and the seventh combination polynucleotide sequence comprises the polynucleotide sequence of SEQ ID NO: 256 or 276.

In some embodiments, the polycistronic polynucleotide comprises, in order from 5′ to 3′: the third polynucleotide sequence, the second polynucleotide sequence, the fifth polynucleotide sequence, the fourth polynucleotide sequence, and the first polynucleotide sequence.

In some embodiments, the third polynucleotide sequence and the second polynucleotide sequence together comprise a fifth combination polynucleotide sequence that encodes the amino acid sequence of SEQ ID NO: 166; the third polynucleotide sequence, the second polynucleotide sequence, and the fifth polynucleotide sequence together comprise a sixth combination polynucleotide sequence that encodes the amino acid sequence of SEQ ID NO: 163; the fifth polynucleotide sequence and the fourth polynucleotide sequence together comprise an eighth combination polynucleotide sequence that encodes the amino acid sequence of SEQ ID NO: 173; or the third polynucleotide sequence, the second polynucleotide sequence, the fifth polynucleotide sequence, and the fourth polynucleotide sequence together comprise a thirteenth combination polynucleotide sequence that encodes the amino acid sequence of SEQ ID NO: 170.

In some embodiments, the fifth combination polynucleotide sequence comprises the polynucleotide sequence of SEQ ID NO: 234; the sixth combination polynucleotide sequence comprises the polynucleotide sequence of SEQ ID NO: 235; the eighth combination polynucleotide sequence comprises the polynucleotide sequence of SEQ ID NO: 237; or the thirteenth combination polynucleotide sequence comprises the polynucleotide sequence of SEQ ID NO: 242.

In some embodiments, the third polynucleotide sequence, the second polynucleotide sequence, the fifth polynucleotide sequence, and the fourth polynucleotide sequence together comprise a thirteenth combination polynucleotide sequence that encodes the amino acid sequence of SEQ ID NO: 170; and the first polynucleotide sequence encodes the amino acid sequence of SEQ ID NO: 40.

In some embodiments, the third polynucleotide sequence, the second polynucleotide sequence, the fifth polynucleotide sequence, and the fourth polynucleotide sequence together comprise a thirteenth combination polynucleotide sequence that encodes the amino acid sequence of SEQ ID NO: 190; and the first polynucleotide sequence encodes the amino acid sequence of SEQ ID NO: 41 or 42.

In some embodiments, the thirteenth combination polynucleotide sequence comprises the polynucleotide sequence of SEQ ID NO: 242; and the first polynucleotide sequence comprises the polynucleotide sequence of SEQ ID NO: 57.

In some embodiments, the thirteenth combination polynucleotide sequence comprises the polynucleotide sequence of SEQ ID NO: 262; and the first polynucleotide sequence comprises the polynucleotide sequence of SEQ ID NO: 55 or 58.

In some embodiments, the polycistronic polynucleotide comprises, in order from 5′ to 3′: the third polynucleotide sequence, the fourth polynucleotide sequence, the fifth polynucleotide sequence, the second polynucleotide sequence, and the first polynucleotide sequence.

In some embodiments, the third polynucleotide sequence and the fourth polynucleotide sequence together comprise a second combination polynucleotide sequence that encodes the amino acid sequence of SEQ ID NO: 168; the third polynucleotide sequence, the fourth polynucleotide sequence, and the fifth polynucleotide sequence together comprise a third combination polynucleotide sequence that encodes the amino acid sequence of SEQ ID NO: 161; the fifth polynucleotide sequence and the second polynucleotide sequence together comprise an eleventh combination polynucleotide sequence that encodes the amino acid sequence of SEQ ID NO: 172; or the third polynucleotide sequence, the fourth polynucleotide sequence, the fifth polynucleotide sequence, and the second polynucleotide sequence together comprise a fourteenth combination polynucleotide sequence that encodes the amino acid sequence of SEQ ID NO: 171.

In some embodiments, the second combination polynucleotide sequence comprises the polynucleotide sequence of SEQ ID NO: 231; the third combination polynucleotide sequence comprises the polynucleotide sequence of SEQ ID NO: 232; the eleventh combination polynucleotide sequence comprises the polynucleotide sequence of SEQ ID NO: 240; or the fourteenth combination polynucleotide sequence comprises the polynucleotide sequence of SEQ ID NO: 243.

In some embodiments, the third polynucleotide sequence, the fourth polynucleotide sequence, the fifth polynucleotide sequence, and the second polynucleotide sequence together comprise a fourteenth combination polynucleotide sequence that encodes the amino acid sequence of SEQ ID NO: 171; and the first polynucleotide sequence encodes the amino acid sequence of SEQ ID NO: 40.

In some embodiments, the third polynucleotide sequence, the fourth polynucleotide sequence, the fifth polynucleotide sequence, and the second polynucleotide sequence together comprise a fourteenth combination polynucleotide sequence that encodes the amino acid sequence of SEQ ID NO: 191; and the first polynucleotide sequence encodes the amino acid sequence of SEQ ID NO: 41 or 42.

In some embodiments, the fourteenth combination polynucleotide sequence comprises the polynucleotide sequence of SEQ ID NO: 243; and the first polynucleotide sequence comprises the polynucleotide sequence of SEQ ID NO: 57.

In some embodiments, the fourteenth combination polynucleotide sequence comprises the polynucleotide sequence of SEQ ID NO: 263; and the first polynucleotide sequence comprises the polynucleotide sequence of SEQ ID NO: 55 or 58.

In some embodiments, the polycistronic polynucleotide comprises, in order from 5′ to 3′: the fifth polynucleotide sequence, the second polynucleotide sequence, the first polynucleotide sequence, the fourth polynucleotide sequence, and the third polynucleotide sequence.

In some embodiments, the first polynucleotide sequence and the fourth polynucleotide sequence together comprise a fourth combination polynucleotide sequence that encodes the amino acid sequence of SEQ ID NO: 162; or the fifth polynucleotide sequence and the second polynucleotide sequence together comprise an eleventh combination polynucleotide sequence that encodes the amino acid sequence of SEQ ID NO: 172.

In some embodiments, the fourth combination polynucleotide sequence comprises the polynucleotide sequence of SEQ ID NO: 233; or the eleventh combination polynucleotide sequence comprises the polynucleotide sequence of SEQ ID NO: 240.

In some embodiments, the first polynucleotide sequence and the fourth polynucleotide sequence together comprise a fourth combination polynucleotide sequence that encodes the amino acid sequence of SEQ ID NO: 162; the fifth polynucleotide sequence and the second polynucleotide sequence together comprise an eleventh combination polynucleotide sequence that encodes the amino acid sequence of SEQ ID NO: 172; and the third polynucleotide sequence encodes the amino acid sequence of SEQ ID NO: 50.

In some embodiments, the first polynucleotide sequence and the fourth polynucleotide sequence together comprise a fourth combination polynucleotide sequence that encodes the amino acid sequence of SEQ ID NO: 182 or 212; the fifth polynucleotide sequence and the second polynucleotide sequence together comprise an eleventh combination polynucleotide sequence that encodes the amino acid sequence of SEQ ID NO: 222; and the third polynucleotide sequence encodes the amino acid sequence of SEQ ID NO: 51.

In some embodiments, the fourth combination polynucleotide sequence comprises the polynucleotide sequence of SEQ ID NO: 233; and the eleventh combination polynucleotide sequence comprises the polynucleotide sequence of SEQ ID NO: 240; and the third polynucleotide sequence comprises the polynucleotide sequence of SEQ ID NO: 59.

In some embodiments, the fourth combination polynucleotide sequence comprises the polynucleotide sequence of SEQ ID NO: 253 or 273; the eleventh combination polynucleotide sequence comprises the polynucleotide sequence of SEQ ID NO: 240; and the third polynucleotide sequence comprises the polynucleotide sequence of SEQ ID NO: 56.

In some embodiments, the polycistronic polynucleotide comprises, in order from 5′ to 3′: the fifth polynucleotide sequence, the fourth polynucleotide sequence, the first polynucleotide sequence, the second polynucleotide sequence, and the third polynucleotide sequence.

In some embodiments, the first polynucleotide sequence and the second polynucleotide sequence together comprise a first combination polynucleotide sequence that encodes the amino acid sequence of SEQ ID NO: 160; or the fifth polynucleotide sequence and the fourth polynucleotide sequence together comprise an eighth combination polynucleotide sequence that encodes the amino acid sequence of SEQ ID NO: 173.

In some embodiments, the first combination polynucleotide sequence comprises the polynucleotide sequence of SEQ ID NO: 230; or the eighth combination polynucleotide sequence comprises the polynucleotide sequence of SEQ ID NO: 237.

In some embodiments, the first polynucleotide sequence and the second polynucleotide sequence together comprise a first combination polynucleotide sequence that encodes the amino acid sequence of SEQ ID NO: 160; the fifth polynucleotide sequence and the fourth polynucleotide sequence together comprise an eighth combination polynucleotide sequence that encodes the amino acid sequence of SEQ ID NO: 173; and the third polynucleotide sequence encodes the amino acid sequence of SEQ ID NO: 50.

In some embodiments, the first polynucleotide sequence and the second polynucleotide sequence together comprise a first combination polynucleotide sequence that encodes the amino acid sequence of SEQ ID NO: 180 or 210; the fifth polynucleotide sequence and the fourth polynucleotide sequence together comprise an eighth combination polynucleotide sequence that encodes the amino acid sequence of SEQ ID NO: 223; and the third polynucleotide sequence encodes the amino acid sequence of SEQ ID NO: 51.

In some embodiments, the first combination polynucleotide sequence comprises the polynucleotide sequence of SEQ ID NO: 230; the eighth combination polynucleotide sequence comprises the polynucleotide sequence of SEQ ID NO: 237; and the third polynucleotide sequence comprises the polynucleotide sequence of SEQ ID NO: 59. In some embodiments, the first combination polynucleotide sequence comprises the polynucleotide sequence of SEQ ID NO: 250 or 270; the eighth combination polynucleotide sequence comprises the polynucleotide sequence of SEQ ID NO: 237; and the third polynucleotide sequence comprises the polynucleotide sequence of SEQ ID NO: 56.

In some embodiments, the polycistronic polynucleotide comprises, in order from 5′ to 3′: the fifth polynucleotide sequence, the second polynucleotide sequence, the third polynucleotide sequence, the fourth polynucleotide sequence, and the first polynucleotide sequence.

In some embodiments, the third polynucleotide sequence and the fourth polynucleotide sequence together comprise a second combination polynucleotide sequence that encodes the amino acid sequence of SEQ ID NO: 168; or the fifth polynucleotide sequence and the second polynucleotide sequence together comprise an eleventh combination polynucleotide sequence that encodes the amino acid sequence of SEQ ID NO: 172.

In some embodiments, the second combination polynucleotide sequence comprises the polynucleotide sequence of SEQ ID NO: 231; or the eleventh combination polynucleotide sequence comprises the polynucleotide sequence of SEQ ID NO: 240.

In some embodiments, the third polynucleotide sequence and the fourth polynucleotide sequence together comprise a second combination polynucleotide sequence that encodes the amino acid sequence of SEQ ID NO: 168; the fifth polynucleotide sequence and the second polynucleotide sequence together comprise an eleventh combination polynucleotide sequence that encodes the amino acid sequence of SEQ ID NO: 172; and the first polynucleotide sequence encodes the amino acid sequence of SEQ ID NO: 40.

In some embodiments, the third polynucleotide sequence and the fourth polynucleotide sequence together comprise a second combination polynucleotide sequence that encodes the amino acid sequence of SEQ ID NO: 188; the fifth polynucleotide sequence and the second polynucleotide sequence together comprise an eleventh combination polynucleotide sequence that encodes the amino acid sequence of SEQ ID NO: 222; and the first polynucleotide sequence encodes the amino acid sequence of SEQ ID NO: 41 or 42.

In some embodiments, the second combination polynucleotide sequence comprises the polynucleotide sequence of SEQ ID NO: 231; the eleventh combination polynucleotide sequence comprises the polynucleotide sequence of SEQ ID NO: 240; and the first polynucleotide sequence comprises the polynucleotide sequence of SEQ ID NO: 57.

In some embodiments, the second combination polynucleotide sequence comprises the polynucleotide sequence of SEQ ID NO: 251; the eleventh combination polynucleotide sequence comprises the polynucleotide sequence of SEQ ID NO: 240; and the first polynucleotide sequence comprises the polynucleotide sequence of SEQ ID NO: 55 or 58.

In some embodiments, the polycistronic polynucleotide comprises, in order from 5′ to 3′: the fifth polynucleotide sequence, the fourth polynucleotide sequence, the third polynucleotide sequence, the second polynucleotide sequence, and the first polynucleotide sequence.

In some embodiments, the third polynucleotide sequence and the second polynucleotide sequence together comprise a fifth combination polynucleotide sequence that encodes the amino acid sequence of SEQ ID NO: 166; or the fifth polynucleotide sequence and the fourth polynucleotide sequence together comprise an eighth combination polynucleotide sequence that encodes the amino acid sequence of SEQ ID NO: 173.

In some embodiments, the fifth combination polynucleotide sequence comprises the polynucleotide sequence of SEQ ID NO: 234; or the eighth combination polynucleotide sequence comprises the polynucleotide sequence of SEQ ID NO: 237.

In some embodiments, the third polynucleotide sequence and the second polynucleotide sequence together comprise a fifth combination polynucleotide sequence that encodes the amino acid sequence of SEQ ID NO: 166; the fifth polynucleotide sequence and the fourth polynucleotide sequence together comprise an eighth combination polynucleotide sequence that encodes the amino acid sequence of SEQ ID NO: 173; and the first polynucleotide sequence encodes the amino acid sequence of SEQ ID NO: 40.

In some embodiments, the third polynucleotide sequence and the second polynucleotide sequence together comprise a fifth combination polynucleotide sequence that encodes the amino acid sequence of SEQ ID NO: 186; the fifth polynucleotide sequence and the fourth polynucleotide sequence together comprise an eighth combination polynucleotide sequence that encodes the amino acid sequence of SEQ ID NO: 223; and the first polynucleotide sequence encodes the amino acid sequence of SEQ ID NO: 41 or 42.

In some embodiments, the fifth combination polynucleotide sequence comprises the polynucleotide sequence of SEQ ID NO: 234; the eighth combination polynucleotide sequence comprises the polynucleotide sequence of SEQ ID NO: 237; and the first polynucleotide sequence comprises the polynucleotide sequence of SEQ ID NO: 57.

In some embodiments, the fifth combination polynucleotide sequence comprises the polynucleotide sequence of SEQ ID NO: 254; the eighth combination polynucleotide sequence comprises the polynucleotide sequence of SEQ ID NO: 237; and the first polynucleotide sequence comprises the polynucleotide sequence of SEQ ID NO: 55 or 58.

In some embodiments, the polycistronic polynucleotide further comprises a sixth polynucleotide sequence that comprises a third 2A element; and a seventh polynucleotide sequence that comprises a marker protein.

In some embodiments, the third 2A element is a P2A element, a T2A element, an F2A element, or an E2A element.

In some embodiments, the marker protein comprises domain III of HER1, or a functional fragment or functional variant thereof, an N-terminal portion of domain IV of HER1; and a transmembrane domain of CD28, or a functional fragment or functional variant thereof.

In some embodiments, the domain III of HER1, or a functional fragment or functional variant thereof, comprises an amino acid sequence at least 90%, 91%, 92%, 93%, 94%, 95%, 96%, 97%, 98%, 99%, or 100% identical to the amino acid sequence of SEQ ID NO: 104.

In some embodiments, the N-terminal portion of domain IV of HER1 comprises amino acids 1-40, 1-39, 1-38, 1-37, 1-36, 1-35, 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, or 1-10 of SEQ ID NO: 105.

In some embodiments, the N-terminal portion of domain IV of HER1 comprises amino acids 1-21 of SEQ ID NO: 105.

In some embodiments, the N-terminal portion of domain IV of HER1 comprises the amino acid sequence of SEQ ID NO: 106, or the amino acid sequence of SEQ ID NO: 106, comprising 1, 2, or 3 amino acid modifications.

In some embodiments, the transmembrane region of CD28 comprises the amino acid sequence of SEQ ID NO: 107, or the amino acid sequence of SEQ ID NO: 107, comprising 1, 2, or 3 amino acid modifications.

In some embodiments, the marker protein comprises an amino acid sequence at least 90%, 91%, 92%, 93%, 94%, 95%, 96%, 97%, 98%, 99%, or 100% identical to the amino acid sequence of SEQ ID NO: 100, 103, or 112.

In some embodiments, the Vα region comprises complementarity determining region 1α (CDR1α), CDR2α, and CDR3α, comprising the amino acid sequences of SEQ ID NO: 1001+10n, 1002+10n, and 1003+10n, respectively, wherein n is an integer from 0 to 79. In some embodiments, n=0.

In some embodiments, the Vβ region comprises CDR1β, CDR2β, and CDR3β, comprising the amino acid sequences of SEQ ID NO: 2001+10n, 2002+10n, and 2003+10n, respectively, wherein n is an integer from 0 to 79. In some embodiments, n=0.

In some embodiments, the Vα region comprises the CDR1α, CDR2α, and CDR3α from a Vα region comprising the amino acid sequence of SEQ ID NO: 1004+10n, 1005+10n, 1006+10n, or 1007+10n, wherein n is an integer from 0 to 79. In some embodiments, n=0.

In some embodiments, the Vβ region comprises the CDR1β, CDR2β, and CDR3β from a Vβ region comprising the amino acid sequence of SEQ ID NO: 2004+10n, 2005+10n, 2006+10n, or 2007+10n, wherein n is an integer from 0 to 79. In some embodiments, n=0.

In some embodiments, the Vα region comprises an amino acid sequence at least 90%, 91%, 92%, 93%, 94%, 95%, 96%, 97%, 98%, 99%, or 100% identical to the amino acid sequence of SEQ ID NO: 1004+10n, 1005+10n, 1006+10n, or 1007+10n, wherein n is an integer from 0 to 79. In some embodiments, n=0.

In some embodiments, the Vβ region comprises an amino acid sequence at least 90%, 91%, 92%, 93%, 94%, 95%, 96%, 97%, 98%, 99%, or 100% identical to the amino acid sequence of SEQ ID NO: 2004+10n, 2005+10n, 2006+10n, or 2007+10n, wherein n is an integer from 0 to 79. In some embodiments, n=0.

In some embodiments, the Vα region comprises an amino acid sequence at least 90%, 91%, 92%, 93%, 94%, 95%, 96%, 97%, 98%, 99%, or 100% identical to the amino acid sequence of SEQ ID NO: 1004+10n, wherein the Vβ region comprises an amino acid sequence at least 90%, 91%, 92%, 93%, 94%, 95%, 96%, 97%, 98%, 99%, or 100% identical to the amino acid sequence of SEQ ID NO: 2004+10n, and wherein n is an integer from 0 to 79; wherein the Vα region comprises an amino acid sequence at least 90%, 91%, 92%, 93%, 94%, 95%, 96%, 97%, 98%, 99%, or 100% identical to the amino acid sequence of SEQ ID NO: 1005+10n, wherein the Vβ region comprises an amino acid sequence at least 90%, 91%, 92%, 93%, 94%, 95%, 96%, 97%, 98%, 99%, or 100% identical to the amino acid sequence of SEQ ID NO: 2005+10n, and wherein n is an integer from 0 to 79; wherein the Vα region comprises an amino acid sequence at least 90%, 91%, 92%, 93%, 94%, 95%, 96%, 97%, 98%, 99%, or 100% identical to the amino acid sequence of SEQ ID NO: 1006+10n, wherein the Vβ region comprises an amino acid sequence at least 90%, 91%, 92%, 93%, 94%, 95%, 96%, 97%, 98%, 99%, or 100% identical to the amino acid sequence of SEQ ID NO: 2006+10n, and wherein n is an integer from 0 to 79; wherein the Vα region comprises an amino acid sequence at least 90%, 91%, 92%, 93%, 94%, 95%, 96%, 97%, 98%, 99%, or 100% identical to the amino acid sequence of SEQ ID NO: 1007+10n, wherein the Vβ region comprises an amino acid sequence at least 90%, 91%, 92%, 93%, 94%, 95%, 96%, 97%, 98%, 99%, or 100% identical to the amino acid sequence of SEQ ID NO: 2007+10n, wherein n is an integer from 0 to 79. In some embodiments, n=0.

In some embodiments, the TCR alpha chain comprises an amino acid sequence at least 90%, 91%, 92%, 93%, 94%, 95%, 96%, 97%, 98%, 99%, or 100% identical to the amino acid sequence of SEQ ID NO: 1008+10n, wherein n is an integer from 0 to 79. In some embodiments, the TCR alpha chain comprises an amino acid sequence at least 90%, 91%, 92%, 93%, 94%, 95%, 96%, 97%, 98%, 99%, or 100% identical to the amino acid sequence of SEQ ID NO: 1009+10n, wherein n is an integer from 0 to 79.

In some embodiments, the TCR alpha chain comprises an amino acid sequence at least 90%, 91%, 92%, 93%, 94%, 95%, 96%, 97%, 98%, 99%, or 100% identical to the amino acid sequence of SEQ ID NO: 1010+10n, wherein n is an integer from 0 to 79.

In some embodiments, the TCR beta chain comprises an amino acid sequence at least 90%, 91%, 92%, 93%, 94%, 95%, 96%, 97%, 98%, 99%, or 100% identical to the amino acid sequence of SEQ ID NO: 2008+10n, wherein n is an integer from 0 to 79. In some embodiments, n=0.

In some embodiments, the TCR beta chain comprises an amino acid sequence at least 90%, 91%, 92%, 93%, 94%, 95%, 96%, 97%, 98%, 99%, or 100% identical to the amino acid sequence of SEQ ID NO: 2009+10n, wherein n is an integer from 0 to 79.

In some embodiments, the TCR beta chain comprises an amino acid sequence at least 90%, 91%, 92%, 93%, 94%, 95%, 96%, 97%, 98%, 99%, or 100% identical to the amino acid sequence of SEQ ID NO: 2010+10n, wherein n is an integer from 0 to 79.

In some embodiments, transcriptional regulatory element comprises a promoter.

In some embodiments, the promoter is a human elongation factor 1-alpha (hEF-1α) hybrid promoter.

In some embodiments, the promoter comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO:150.

In some embodiments, the recombinant vector further comprises a polyA sequence at the 3′ end of the polycistronic expression cassette.

In some embodiments, the polyA sequence comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO:151.

In some embodiments, the recombinant vector further comprises a Left inverted terminal repeat (ITR) and a Right ITR, wherein the Left ITR and the Right ITR flank the polycistronic expression cassette.

In some embodiments, the recombinant vector comprises, in order from 5′ to 3′: the Left ITR; the transcriptional regulatory element; the first polynucleotide sequence; the second polynucleotide sequence; the third polynucleotide sequence; the fourth polynucleotide sequence; the fifth polynucleotide sequence; and the Right ITR.

In some embodiments, the recombinant vector is a non-viral vector.

In some embodiments, the non-viral vector is a plasmid.

In some embodiments, the recombinant vector is a viral vector.

In some embodiments, the recombinant vector is a polynucleotide.

Also provided herein is a polynucleotide encoding an amino acid sequence at least 90%, 91%, 92%, 93%, 94%, 95%, 96%, 97%, 98%, 99%, or 100% identical to an amino acid sequence selected from the group consisting of SEQ ID NOs: 161, 163, 164, 165, 167, 169, 170, and 171.

Also provided herein is a polynucleotide comprising a polynucleotide sequence at least 75%, 80%, 85%, 90%, 91%, 92%, 93%, 94%, 95%, 96%, 97%, 98%, 99%, or 100% identical to a polynucleotide sequence selected from the group consisting of SEQ ID NOs: 232, 235, 236, 238, 239, 241, 242, and 243.

Also provided herein is a population of cells that comprises any recombinant vector provided herein, or any polynucleotide provided herein.

In some embodiments, the recombinant vector or the polynucleotide is integrated into the genome of the population of cells.

In some embodiments, the cells are immune effector cells.

In some embodiments, the immune effector cells are selected from the group consisting of T cells, natural killer (NK) cells, B cells, mast cells, and myeloid-derived phagocytes.

In some embodiments, the immune effector cells are T cells.

In some embodiments, the T cells are selected from the group consisting of naïve T cells (CD4+ or CD8+); killer CD8+ T cells; cytotoxic CD4+ T cells; helper CD4+ T cells; CD4+ T cells corresponding to Th1, Th2, Th9, Th17, Th22, follicular helper (Th), regulatory (Treg) lineages; tumor infiltrating lymphocytes (TILs); and memory T cells (central memory, effector memory, stem cell memory, stem cell-like memory).

In some embodiments, the population of cells comprises alpha/beta T cells, gamma/delta T cells, or natural killer T (NKT) cells.

In some embodiments, the population of cells comprises CD4⁺ T cells, CD8⁺ T cells, or both CD4⁺ T cells and CD8⁺ T cells.

In some embodiments, the cells are ex vivo.

In some embodiments, the cells are human.

In some embodiments, the population of cells are T cells that comprise more than 5%, 10%, 15%, 20%, 25%, 30%, 35%, 40%, 45% or 50% CD45RA+CD45RO−CD62L+CD95+ cells.

In some embodiments, the population of cells are T cells that comprise more than 5%, 10%, 15%, 20%, 25%, 30%, 35%, 40%, 45% or 50% CD45RA+CD45RO+CD62L+CD95+ cells.

Also provided herein is a population of cells comprising a polycistronic expression cassette comprising a first cistron comprising a polynucleotide sequence that encodes a fusion protein that comprises IL-15, or a functional fragment or functional variant thereof, and IL-15Rα, or a functional fragment or functional variant thereof, a second cistron comprising a polynucleotide sequence that encodes a TCR beta chain comprising a Vβ region and a Cβ region; and a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO−CD62L+CD95+ cells.

Also provided herein is a population of cells comprising a polycistronic expression cassette comprising a first cistron comprising a polynucleotide sequence that encodes a fusion protein that comprises IL-15, or a functional fragment or functional variant thereof, and IL-15Rα, or a functional fragment or functional variant thereof, a second cistron comprising a polynucleotide sequence that encodes a TCR beta chain comprising a Vβ region and a Cβ region; and a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO+CD62L+CD95+ cells.

Also provided herein is a method of producing a population of engineered cells, comprising introducing into a population of cells any recombinant vector provided herein, and a DNA transposase or a polynucleotide encoding a DNA transposase; and culturing the population of cells under conditions wherein the transposase integrates the polycistronic expression cassette into the genome of the population of cells, thereby producing the population of engineered cells.

In some embodiments, the Left ITR and the Right ITR are ITRs of a DNA transposon selected from the group consisting of a Sleeping Beauty transposon, a piggyBac transposon, a TcBuster transposon, and a Tol2 transposon.

In some embodiments, the DNA transposon is the Sleeping Beauty transposon.

In some embodiments, the transposase is a Sleeping Beauty transposase.

In some embodiments, the Sleeping Beauty transposase is selected from the group consisting of SB11, SB10, SB100X, hSB110, and hSB81.

In some embodiments, the Sleeping Beauty transposase is SB11.

In some embodiments, the SB11 comprises an amino acid sequence at least 90%, 91%, 92%, 93%, 94%, 95%, 96%, 97%, 98%, 99%, or 100% identical to the amino acid sequence of SEQ ID NO: 300.

In some embodiments, the SB11 is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 301.

In some embodiments, the polynucleotide encoding the DNA transposase is a DNA vector or an RNA vector.

In some embodiments, the Left ITR comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 290 or 291; and the Right ITR comprises a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 292, 293 or 294.

In some embodiments, the recombinant vector, and the DNA transposase or polynucleotide encoding the DNA transposase, are introduced to the population of cells using electroporation, sonication, calcium phosphate precipitation, lipofection, particle bombardment, microinjection, or mechanical deformation by passage through a microfluidic device, or a colloidal dispersion system.

In some embodiments, the recombinant vector, and the DNA transposase or polynucleotide encoding the DNA transposase, are introduced to the population of cells using electroporation.

In some embodiments, the method is completed within 30 days, 25 days, 20 days, 15 days, 14 days, 10 days, 7 days, 6 days, 5 days, 4 days, 3 days, 2 days, or 1 day.

In some embodiments, wherein the method is completed in less than 30 days, 25 days, 20 days, 15 days, 14 days, 10 days, 7 days, 6 days, 5 days, 4 days, 3 days, 2 days, or 1 day.

In some embodiments, the population of cells is cryopreserved and thawed before introduction of the recombinant vector and the DNA transposase or polynucleotide encoding the DNA transposase.

In some embodiments, the population of cells is rested before introduction of the recombinant vector and the DNA transposase or polynucleotide encoding the DNA transposase.

In some embodiments, the population of cells is not rested before introduction of the recombinant vector and the DNA transposase or polynucleotide encoding the DNA transposase.

In some embodiments, the population of cells comprises expanded human ex vivo cells.

In some embodiments, the population of cells is not activated ex vivo.

In some embodiments, the population of cells comprises T cells.

Also provided herein is a method of treating cancer in a subject in need thereof comprising administering to the subject a therapeutically effective amount of any of the populations of cells provided herein, thereby treating the cancer.

In some embodiments, the cancer is selected from lung, cholangiocarcinoma, pancreatic, colorectal, gynecological, and ovarian cancer.

Also provided herein is a method of treating an autoimmune disease or disorder in a subject in need thereof comprising administering to the subject a therapeutically effective amount any of the populations of cells provided herein, thereby treating the autoimmune disease or disorder.

### 4. BRIEF DESCRIPTION OF THE DRAWINGS

The accompanying drawings which are incorporated in and constitute a part of this specification, illustrate exemplary embodiments of the present disclosure and, together with the description, serve to explain principles of the present disclosure.

FIG. 1 is a set of schematics of the structures of TCRα (A), TCRβ (B), and mbIL15 (15), shown from N terminus (left) to C terminus (right).

FIG. 2A is a set of schematics of the ORFs of tricistronic Cassettes APBT15, ATBP15, AP15 TB, AT15PB, BPAT15, BTAP15, BP15TA, and BT15PA. FIG. 2B is a set of schematics of the ORFs of control Cassettes 15, APB, and BPA.

FIG. 3 is a schematic diagram depicting double transposition and single transposition approaches using a Sleeping Beauty transposon/transposase system to generate T cells expressing TCRα/TCRβ and mbIL15.

FIG. 4 is a set of 2-parameter flow plots showing transgene co-expression as assessed after electroporation and overnight incubation for each of Groups 1-14.

FIG. 5A is a set of 2-parameter flow plots showing representative TCR transgene expression in CD3+ cells after overnight incubation for each of Groups 1-14. FIG. 5B provides TCR expression data from three donors presented as % mTCR+ cells out of CD3+ cells.

FIG. 6A-6C shows TCR and mbIL15 expression after first phase expansion (Day 13). FIG. 6A provides representative TCR and mbIL15 expression data from each of Groups 1-14. FIG. 6B provides TCR expression data from three donors presented as % mTCR+ cells out of CD3+ cells. FIG. 6C provides TCR and mbIL15 co-expression data from three donors presented as % TCR+mbIL15+ cells out of CD3+ cells.

FIG. 7A-7B shows total numbers of TCR+ and TCR+mbIL15+ cells after first phase expansion (Day 13). FIG. 7A provides TCR expression data from three donors presented as total number of mTCR+ T cells. FIG. 7B provides total number of TCR+mbIL15+ T cells from three donors.

FIG. 8A-8B shows cell viability after electroporation (Day 1; FIG. 8A) and after first phase expansion (Day 13; FIG. 8B) for each of Groups 1-14.

FIG. 9A-9B shows specific induction of activation marker, 4-1BB, after overnight co-culture of transposed T cells from each of Groups 1-14 after first phase expansion (Day 13) with wild-type or mutant neoantigen pulsed T2 cells. Data is presented as % 4-1BB positive cells of CD8+ cells at increasing concentrations of neoantigen peptide.

FIG. 10 shows phosphorylated STAT5 levels in transposed CD3+ T cells from each of Groups 1-14 after first phase expansion (Day 13). Isotype negative control and IL-15 treated positive control was included for comparison. (dTp=double transposed with separate mbIL15 and TCR vectors).

FIG. 11 shows apoptosis levels in transposed T cells from each of Groups 2-14 after being expanded for 13 days and then activated for 9 days with CD3/CD28 Dynabeads® (ThermoFisher).

FIG. 12 is a set of schematics illustrating the differences between the S version and N version of the TCR only and mbIL15 TCR constructs shown from N terminus (left) to C terminus (right).

FIG. 13A-13B shows TCR expression on CD3+ cells (FIG. 13A) the day after electroporation (Day 1) and after the first phase expansion prior to enrichment (Day 11 Pre-enrichment) as well as (FIG. 13B) after the first phase expansion following enrichment (Day 11 Post-enrichment) and at the end of the second phase expansion. Data from four donors are presented.

FIG. 14A-14B shows TCR and mbIL15 co-expression on CD3+ T cells (FIG. 14A) the day after electroporation (Day 1) and after the first phase expansion prior to enrichment (Day 11 Pre-enrichment) as well as after the first phase expansion following enrichment (Day 11 Post-enrichment) and at the end of the second phase expansion. Data from four donors are presented.

FIG. 15 is a set of 2-parameter flow plots showing representative TCR and mbIL15 transgenes expression in CD3+ cells after the second phase expansion.

FIG. 16A-16B shows the fold expansion of cells (FIG. 16A) after the first expansion phase and (FIG. 16B) after the second expansion phase. Data from four donors are presented.

FIG. 17A-17B shows the absolute count of TCR expressing cells in culture (FIG. 17A) after the first expansion phase and (FIG. 17B) after the second expansion phase. Data from four donors are presented.

FIG. 18 shows phosphorylated STAT5 levels after second phase expansion (Day 27) in CD3+ T cells transposed with different versions of polycistronic plasmids encoding TCR001. Some containing non-cysteine substituted TCR constant regions (N version) or that are optionally further codon-optimized (NU version). Non-transposed (NT)=NT (Group 2.1); BPA (Group 2.2); BPA-N (Group 2.3); AP15 TB (Group 2.4); AP15 TB-N (Group 2.5); AP15 TB-NU (Group 2.6); BP15TA (Group 2.7); BP15TA-N (Group 2.8); and BP15TA-NU (Group 2.9).

FIG. 19A-19B shows functional data from transposed T cells co-cultured with neoantigen pulsed dendritic cells. FIG. 19A shows specific induction of activation marker, 4-1BB, after overnight co-culture of transposed T cells from each of Groups 2.1-2.9 after second phase expansion (Day 27) with wild-type or mutant neoantigen peptide pulsed dendritic cells. Data is presented as % 4-1BB positive cells of CD8+ cells at increasing concentrations of neoantigen peptide. FIG. 19B shows interferon-7 (IFN-γ) secretion after overnight co-culture of transposed T cells from each of Groups 2.1-2.9 after second phase expansion (Day 27) with wild-type or mutant neoantigen pulsed dendritic cells.

FIG. 20A-20B shows TCR expression and cell survival after 4 weeks of long-term cytokine withdrawal (LTWD) incubation in transposed cells from each of Groups 2.2-2.9. FIG. 20A shows the expression of mTCR detected on CD3+ gated population with mouse TCR beta antibody and FIG. 20B shows cell survival as the percent of live cells recovered relative to initial input number of cells at the beginning of the LTWD.

FIG. 21A-21B shows specific induction of activation marker, 4-1BB, after overnight co-culture of transposed T cells from each of Groups 2.2-2.9 after 4 weeks of LTWD incubation with wild-type or mutant neoantigen (10 μg/ml) pulsed dendritic cells.

FIG. 22A-22B shows IFN-γ secretion after overnight co-culture of transposed T cells from each of Groups 2.2-2.9 after 4 weeks of LTWD incubation with wild-type or mutant neoantigen (10 μg/ml) pulsed dendritic cells.

FIG. 23A-23C is a set of pie charts showing the mean frequency of live CD3⁺ T cell memory and effector subsets at day 11 post-expansion (FIG. 23A), day 22 post-expansion (FIG. 23B), and after 4 weeks of LTWD culture (FIG. 23C) in cells transposed with the tested plasmids (Groups 2.2-2.9).

FIG. 24 is a set of 2-parameter flow plots showing representative TCR and mbIL15 transgene co-expression in CD3+ cells after overnight incubation (Day 1), after first phase expansion (Day 11, pre- and post-enrichment) and after second phase expansion (Day 22) for each of Groups 3.2-3.4 expressing TCR001+/−mbIL15 (BPA-N, AP15 TB-NU, and BP15TA-NU).

FIG. 25A-25C shows TCR+ population changes during the first expansion phase (Day 1 vs. Day 11 pre-enrichment) for cells transposed with various TCRs+/−mbIL15 (Groups 3.1-3.30). Each graph presents data from a separate TCR; TCR only=BPA-N, TCR with mbIL15=AP15 TB-NU or BP15TA-NU.

FIG. 26A-26C shows TCR+ population changes during the second expansion phase (Day 11 post-enrichment vs. Day 22) for cells transposed with various TCRs+/−mbIL15 (Groups 3.1-3.30). Each graph presents data from a separate TCR; TCR only=BPA-N, TCR with mbIL15=AP15 TB-NU or BP15TA-NU.

FIG. 27A-27C shows TCR+/mbIL15+ population changes during the first expansion phase (Day 1 vs. Day 11 pre-enrichment) for cells transposed with various TCRs+/−mbIL15 (Groups 3.1-3.30). Each graph presents data from a separate TCR; TCR only=BPA-N, TCR with mbIL15=AP15 TB-NU or BP15TA-NU.

FIG. 28A-28C shows TCR+/mbIL15+ population changes during the second expansion phase (Day 11 post-enrichment vs. Day 22) for cells transposed with various TCRs+/−mbIL15 (Groups 3.1-3.30). Each graph presents data from a separate TCR; TCR only=BPA-N, TCR with mbIL15=AP15 TB-NU or BP15TA-NU.

FIG. 29A-29I shows specific induction of activation marker, 4-1BB, after overnight co-culture of transposed T cells from each of Groups 3.1-3.30 after second phase expansion (Day 27) with wild-type (WT) or mutant (Mut) neoantigen pulsed dendritic cells. Data is presented as % 4-1BB positive cells of total CD3+, CD4+ or CD8+ T cells at increasing concentrations of neoantigen peptide. NT=non-transposed; TCR only=BPA-N, TCR with mbIL15=AP15 TB-NU or BP15TA-NU.

FIG. 30A-30I shows IFN-γ secretion after overnight co-culture of transposed T cells from each of Groups 3.1-3.30 after second phase expansion (Day 27) with wild-type (WT) or mutant (Mut) neoantigen pulsed dendritic cells. Data is presented as IFN-γ level (pg/mL) at increasing concentrations of neoantigen peptide. NT=non-transposed; TCR only=BPA-N, TCR with mbIL15=AP15 TB-NU or BP15TA-NU.

FIG. 31 shows the specific lysis of negative control (Mut+HLA−) tumor cell line AU565 and target tumor cell line TYK-nu (Mut+HLA+) by T cells expressing TCR001+/−mbIL15. NT=non-transposed; TCR001 only=BPA-N, TCR001 with mbIL15=AP15 TB-NU or BP15TA-NU.

FIG. 32A-32B shows the specific lysis of a tumor cell line by T cells expressing (FIG. 32A) TCR022+/−mbIL15 or (FIG. 32B) TCR075+/−mbIL15. Tumor cell line was transfected with the appropriate HLA-expression plasmid and pulsed with either wild type (WT) or mutant (Mut) peptides and co-cultured with T cells. NT=non-transposed; TCR only=BPA-N, TCR with mbIL15=AP15 TB-NU or BP15TA-NU.

FIG. 33 shows TCR+ population for cells transposed with various TCRs+/−mbIL15 (Groups 3.1-3.30) after long-term cytokine withdrawal (LTWD). TCR only=BPA-N, TCR with mbIL15=AP15 TB-NU or BP15TA-NU.

FIG. 34A-34C shows cell survival for cells transposed with various TCRs+/−mbIL15 (Groups 3.1-3.30) after long-term cytokine withdrawal (LTWD). BPA-N (IL2)=TCR only cultured with IL2, NT=non-transposed, BPA-N=TCR only, TCR with mbIL15=AP15 TB-NU or BP15TA-NU.

FIG. 35A-35I shows specific induction of activation marker, 4-1BB, after overnight co-culture of cells transposed with various TCRs+/−mbIL15 (Groups 3.1-3.30) after long-term cytokine withdrawal (LTWD) with wild-type or mutant neoantigen pulsed dendritic cells. Data are presented as % 4-1BB+ of either CD3+, CD4+, or CD8+ T cells. BPA-N (IL2)=TCR only cultured with IL2, TCR with mbIL15=AP15 TB-NU or BP15TA-NU.

FIG. 36A-36C shows IFN-γ secretion after overnight co-culture of cells transposed with various TCRs+/−mbIL15 (Groups 3.1-3.30) after long-term cytokine withdrawal (LTWD) with wild-type or mutant neoantigen pulsed dendritic cells. BPA-N (IL2)=TCR only cultured with IL2, TCR with mbIL15=AP15 TB-NU or BP15TA-NU.

FIG. 37A-37I shows a comparison of 4-1BB induction in cells transposed with various TCRs+mbIL15 (Groups 3.1-3.30) pre- and post-LTWD culture after overnight co-culture with wild-type or mutant neoantigen pulsed dendritic cells. Data are presented as % 4-1BB+ of either CD3+, CD4+, or CD8+ T cells.

FIG. 38 is a set of representative pie charts showing the mean frequency of live CD3⁺ T cell memory and effector subsets at day 11 post-expansion of cells transposed with TCR001 expressed from either BPA-N or with mbIL15 from either AP15 TB-NU or BP15TA-NU.

FIG. 39 is a set of representative pie charts showing the mean frequency of live CD3⁺ T cell memory and effector subsets at day 22 post-expansion of cells transposed with TCR001 expressed from either BPA-N or with mbIL15 from either AP15 TB-NU or BP15TA-NU.

FIG. 40A-40E is a set of pie charts showing the mean frequency of live CD3⁺ T cell memory and effector subsets of in cells transposed with the tested plasmids (Groups 3.1-3.30) after 4 weeks of LTWD culture.

### 5. DETAILED DESCRIPTION

The instant disclosure provides recombinant polycistronic nucleic acid vectors comprising at least three cistrons, wherein the first cistron encodes an α chain of an artificial T-cell receptor (TCR), the second cistron encodes a β chain of an artificial TCR, and the third cistron encodes a fusion protein that comprises IL-15 and IL-15Rα (e.g., mbIL15), or a functional fragment or functional variant thereof. In some embodiments, the polycistronic nucleic acid further comprises a fourth cistron that encodes a marker protein (e.g., HER1t). In some embodiments, the cistrons are separated by polynucleotide sequence that comprise 2A elements. Also provided are immune effector cells comprising these vectors, immune effector cells engineered ex vivo utilizing the vectors to express the three proteins encoded by the vectors, pharmaceutical compositions comprising these vectors or engineered immune effector cells made utilizing these vectors, and methods of treating a subject using these vectors or engineered immune effector cells made utilizing these vectors.

The polycistronic vectors described herein are particularly useful in methods of manufacturing populations of engineered cells (e.g., immune effector cells) that are substantially homogeneous compared to the prior art systems that utilized at least two vectors for the expression of three proteins.

### 5.1 Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the value or range remain within the intended meaning of the recited value or range.

As used herein, the terms “T cell receptor” and “TCR” are used interchangeably and refer to molecules comprising CDRs or variable regions from as T cell receptors. Examples of TCRs include, but are not limited to, full-length TCRs, antigen-binding fragments of TCRs, soluble TCRs lacking transmembrane and cytoplasmic regions, single-chain TCRs containing variable regions of TCRs attached by a flexible linker, TCR chains linked by an engineered disulfide bond, single TCR variable domains, single peptide-MHC-specific TCRs, multi-specific TCRs (including bispecific TCRs), TCR fusions, TCRs comprising co-stimulatory regions, human TCRs, humanized TCRs, chimeric TCRs, recombinantly produced TCRs, and synthetic TCRs. In certain embodiments, the TCR is a full-length TCR comprising a full-length α chain and a full-length β chain. In certain embodiments, the TCR is a soluble TCR lacking transmembrane and/or cytoplasmic region(s). In certain embodiments, the TCR is a single-chain TCR (scTCR) comprising Vα and Vβ linked by a peptide linker, such as a scTCR having a structure as described in PCT Publication No.: WO 2003/020763, WO 2004/033685, or WO 2011/044186, each of which is incorporated by reference herein in its entirety. In certain embodiments, the TCR comprises a transmembrane region. In certain embodiments, the TCR comprises a co-stimulatory signaling region.

As used herein, the term “full-length TCR” refers to a TCR comprising a dimer of a first and a second polypeptide chain, each of which comprises a TCR variable region and a TCR constant region comprising a TCR transmembrane region and a TCR cytoplasmic region. In certain embodiments, the full-length TCR comprises one or two unmodified TCR chains, e.g., unmodified a or PTCR chains. In certain embodiments, the full-length TCR comprises one or two altered TCR chains, such as chimeric TCR chains and/or TCR chains comprising one or more amino acid substitutions, insertions, or deletions relative to an unmodified TCR chain. In certain embodiments, the full-length TCR comprises a mature, full-length TCR α chain and a mature, full-length TCR β chain.

As used herein, the term “TCR variable region” refers to the portion of a mature TCR polypeptide chain (e.g., a TCR α chain or β chain) which is not encoded by the TRAC gene for TCR α chains, either the TRBC1 or TRBC2 genes for TCR β chains, or the TRDC gene for TCR δ chains. In some embodiments, the TCR variable region of a TCR α chain encompasses all amino acids of a mature TCR α chain polypeptide which are encoded by a TRAV and/or TRAJ gene, and the TCR variable region of a TCR β chain encompasses all amino acids of a mature TCR β chain polypeptide which are encoded by a TRBV, TRBD, and/or TRBJ gene (see, e.g., Lefranc and Lefranc, (2001) “T cell receptor FactsBook.” Academic Press, ISBN 0-12-441352-8, which is incorporated by reference herein in its entirety). TCR variable regions generally comprise framework regions (FR) 1, 2, 3, and 4 and complementarity determining regions (CDR) 1, 2, and 3.

As used herein, the terms “α chain variable region” and “Vα” are used interchangeably and refer to the variable region of a TCR α chain.

As used herein, the terms “β chain variable region” and “Vβ” are used interchangeably and refer to the variable region of a TCR β chain.

As used herein in the context of a TCR, the term “CDR” or “complementarity determining region” means the noncontiguous antigen combining sites found within the variable regions of a TCR chain (e.g., an α chain or a β chain). These regions have been described in Lefranc, (1999) The Immunologist 7: 132-136; Lefranc et al., (1999) Nucleic Acids Res 27: 209-212; Lefranc (2001) “T cell receptor FactsBook.” Academic Press, ISBN 0-12-441352-8; Lefranc et al., (2003) Dev Comp Immunol. 27(1):55-77; and in Kabat et al., (1991) “Sequences of protein of immunological interest,” each of which is herein incorporated by reference in its entirety. In certain embodiments, CDRs are determined according to the IMGT numbering system described in Lefranc (1999) supra. In certain embodiments, CDRs are defined according to the Kabat numbering system described in Kabat supra. In certain embodiments, CDRs are defined empirically, e.g., based upon a structural analysis of the interaction of a TCR with a cognate antigen (e.g., a peptide or a peptide-MHC complex). In certain embodiments, the α chain and β chain CDRs of a TCR are defined according to different conventions (e.g., according to the Kabat or IMGT numbering systems, or empirically based upon structural analysis).

As used herein, the term “framework amino acid residues” refers to those amino acids in the framework region of a TCR chain (e.g., an α chain or a β chain). The term “framework region” or “FR” as used herein includes the amino acid residues that are part of the TCR variable region, but are not part of the CDRs.

As used herein, the term “constant region” with respect to a TCR refers to the portion of a TCR that is encoded by the TRAC gene (for TCR α chains) or either the TRBC1 or TRBC2 gene (for TCR β chains), optionally lacking all or a portion of a transmembrane region and/or all or a portion of a cytoplasmic region. In certain embodiments, a TCR constant region lacks a transmembrane region and a cytoplasmic region. A TCR constant region does not include amino acids encoded by a TRAV, TRAJ, TRBV, TRBD, TRBJ, TRDV, TRDD, TRDJ, TRGV, or TRGJ gene (see, e.g., “T cell receptor FactsBook,” supra).

As used herein, the terms “major histocompatibility complex” and “MHC” are used interchangeably and refer to an MHC class I molecule and/or an MHC class II molecule.

As used herein, the term “MHC class I” refers to a dimer of an MHC class I α chain and a β2 microglobulin chain and the term “MHC class II” refers to a dimer of an MHC class II a chain and an MHC class II β chain.

As used herein, the terms “human leukocyte antigen” and “HLA” are used interchangeably and can also refer to the proteins encoded by the MHC genes. HLA-A, HLA-B, HLA-C, HLA-E, HLA-F, and HLA-G refer to major and minor gene products of MHC class I genes. HLA-DP, HLA-DQ, and HLA-DR refer to gene products of MHC class I genes, which are expressed on antigen-presenting cells, B cells, and T cells.

As used herein, the term “peptide-MHC complex” refers to an MHC molecule (MHC class I or MHC class II) with a peptide bound in the art-recognized peptide binding pocket of the MHC. In some embodiments, the MHC molecule is a membrane-bound protein expressed on the cell surface. In some embodiments, the MHC molecule is a soluble protein lacking transmembrane or cytoplasmic regions.

As used herein, the term “extracellular” with respect to a recombinant transmembrane protein refers to the portion or portions of the recombinant transmembrane protein that are located outside of a cell.

As used herein, the term “transmembrane” with respect to a recombinant transmembrane protein refers to the portion or portions of the recombinant transmembrane protein that are embedded in the plasma membrane of a cell.

As used herein, the term “cytoplasmic” with respect to a recombinant transmembrane protein refers to the portion or portions of the recombinant transmembrane protein that are located in the cytoplasm of a cell.

As used herein, the term “co-stimulatory signaling region” refers to the intracellular portion of a co-stimulatory molecule that is responsible for mediating intracellular signaling events.

“Binding affinity” generally refers to the strength of the sum total of non-covalent interactions between a single binding site of a molecule (e.g., a TCR) and its binding partner (e.g., a peptide-MHC complex). Unless indicated otherwise, as used herein, “binding affinity” refers to intrinsic binding affinity which reflects a 1:1 interaction between members of a binding pair (e.g., a TCR and a peptide-MHC complex). The affinity of a molecule X for its partner Y can generally be represented by the dissociation constant (K_{D}). Affinity can be measured and/or expressed in a number of ways known in the art, including, but not limited to, equilibrium dissociation constant (K_{D}) and equilibrium association constant (K_{A}). The K_{D} is calculated from the quotient of k_{off}/kₒₙ, whereas K_{A} is calculated from the quotient of kₒₙ/k_{off}. Kₒₙ refers to the association rate constant and k_{off} refers to the dissociation rate constant. The kₒₙ and k_{off} can be determined by techniques known to one of ordinary skill in the art, such as use of BIAcore® or KinExA. As used herein, a “lower affinity” refers to a larger K_{D}.

“Avidity” generally refers to the affinity of binding molecule (e.g., a TCR) and its binding partner (e.g., a peptide-MHC complex). Binding molecules described herein are able to bind antigen via two (or more) sites in which the multiple interactions synergize to enhance the “apparent” affinity. Avidity is the measure of the strength of binding between the binding molecule described herein (e.g., a TCR) and the pertinent antigens (e.g., a peptide-MHC complex). Avidity is related to both the affinity between an antigenic determinant and its antigen binding site on the antigen-binding molecule and the number of pertinent binding sites present on the antigen-binding molecules.

For example, “specifically binds to” may be used to refer to the ability of a TCR to preferentially bind to a particular antigen (e.g., a specific peptide or a specific peptide-MHC complex combination) as such binding is understood by one skilled in the art. For example, a TCR that specifically binds to an antigen can bind to other antigens, generally with lower affinity as determined by, e.g., BIAcore®, or other immunoassays known in the art (see, e.g., Savage et al., (1999) Immunity. 10(4):485-92, which is incorporated by reference herein in its entirety). In a specific embodiment, a TCR that specifically binds to an antigen binds to the antigen with an association constant (Kₐ) that is at least 2-fold, 5-fold, 10-fold, 50-fold, 100-fold, 500-fold, 1,000-fold, 5,000-fold, or 10,000-fold greater than the Kₐ when the TCR binds to another antigen.

As used herein, an “epitope” is a term in the art and refers to a localized region of an antigen (e.g., a peptide or a peptide-MHC complex) to which a TCR can bind. In certain embodiments, the epitope to which a TCR binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), flow cytometry analysis, mutagenesis mapping (e.g., site-directed mutagenesis mapping), and/or structural modeling. For X-ray crystallography, crystallization may be accomplished using any of the known methods in the art (e.g., Giege R et al., (1994) Acta Crystallogr D Biol Crystallogr 50(Pt 4): 339-350; McPherson A, (1990) Eur J Biochem 189: 1-23; Chayen N E, (1997) Structure 5: 1269-1274; McPherson A, (1976) J Biol Chem 251: 6300-6303, each of which is herein incorporated by reference in its entirety). TCR:antigen crystals may be studied using well-known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H. W., et al.; U.S. 2004/0014194); and BUSTER (Bricogne G, (1993) Acta Crystallogr D Biol Crystallogr 49(Pt 1): 37-60; Bricogne G, (1997) Meth Enzymol 276A: 361-423, ed Carter C W; and Roversi P et al., (2000) Acta Crystallogr D Biol Crystallogr 56(Pt 10): 1316-1323), each of which is herein incorporated by reference in its entirety. Mutagenesis mapping studies may be accomplished using any method known to one of skill in the art. See, e.g., Champe M et al., (1995)J Biol Chem 270: 1388-1394 and Cunningham B C & Wells J A, (1989) Science 244: 1081-1085, each of which is herein incorporated by reference in its entirety, for a description of mutagenesis techniques, including alanine scanning mutagenesis techniques. In a specific embodiment, the epitope of an antigen is determined using alanine scanning mutagenesis studies. In a specific embodiment, the epitope of an antigen is determined using hydrogen/deuterium exchange coupled with mass spectrometry. In certain embodiments, the antigen is a peptide-MHC complex. In certain embodiments, the antigen is a peptide presented by an MHC molecule.

As used herein, the terms “treat,” “treating,” and “treatment” refer to therapeutic or preventative measures described herein. In some embodiments, the methods of“treatment” employ administration of a TCR or a cell expressing a TCR to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In one embodiment, the subject is a human or non-human mammal. In one embodiment, the subject is a human.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87: 2264-2268, modified as in Karlin S & Altschul S F, (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody.

As used herein, the term “cistron” refers to a polynucleotide sequence from which a transgene product can be produced.

As used herein, the term “polycistronic vector” refers to a polynucleotide vector that comprises a polycistronic expression cassette.

As used herein, the term “polycistronic expression cassette” refers to a polynucleotide sequence wherein the expression of three or more transgenes is regulated by common transcriptional regulatory elements (e.g., a common promoter) and can simultaneously express three or more separate proteins from the same mRNA. Exemplary polycistronic vectors, without limitation, include tricistronic vectors (containing three cistrons) and tetracistronic vectors (containing four cistrons).

As used herein, the term “polycistronic polynucleotide” refers to a polynucleotide that comprises three or more cistrons.

As used herein, the term “transcriptional regulatory element” refers to a polynucleotide sequence that mediates regulation of transcription of another polynucleotide sequence. Exemplary transcriptional regulatory elements include, but are not limited to, promoters and enhancers.

As used herein, a “furin recognition site” refers to an amino acid sequence, or a nucleotide sequence encoding the amino acid sequence, which can be cleaved by the furin enzyme. The furin enzyme is also known as PACE. In some embodiments, the furin recognition site comprises the amino acid sequence RXXR (SEQ ID NO: 1), wherein X at position 2 is any amino acid and X at position 3 is arginine or lysine. In some embodiments, the furin recognition site comprises the sequences shown below in Table 1.

TABLE 1
Amino acid sequences of exemplary
furin recognition sites and
polynucleotide sequences encoding same.

| | | SEQ |
|---|---|---|
| Description | Sequence | ID NO: |
| Furin recognition site | RAKR | 2 |
| Furin recognition site | CGGGCGAAACGC | 3 |
| polynucleotide coding | | |
| sequence | | |
| Furin recognition site | RAKRSGSG | 4 |
| (alternative) | | |
| Furin recognition site | CGGGCGAAACGC | 5 |
| (alternative) | TCTGGAAGCGGA | |
| polynucleotide coding | | |
| sequence | | |

In some embodiments, the furin recognition site comprises an amino acid sequence that is identical to the amino acid sequence of SEQ ID NO: 2 or 4, or comprises 1, 2, or 3 amino acid modifications, relative to SEQ ID NO: 2 or 4; or is encoded by a polynucleotide sequence 75%, 80%, 85%, 90%, 95%, 96%, 97%, 98%, 99%, or 100% identical to the polynucleotide sequence of SEQ ID NO: 3 or 5. In some embodiments, when positioned in a vector between a first polynucleotide sequence encoding a first protein and a second polynucleotide sequence encoding a second protein, the furin recognition site is capable of mediating the cleavage (via furin) of the first protein from the second protein, resulting in two distinct polypeptides from the same mRNA molecule.

Responsive to recognition of the furin recognition site by the furin enzyme, the furin enzyme induces cleavage of a given polypeptide on the C-terminal side of the furin recognition site or a portion thereof. Accordingly, polypeptides produced by furin-mediated cleavage at a furin recognition site may retain all or a portion of the furin recognition site on their C-terminus. For example, the C-terminus of a first polypeptide of the present disclosure may comprise the amino acid sequence RAKR (SEQ ID NO: 2) or RA.

As used herein, a “2A element” refers to a polynucleotide sequence which, when expressed in an mRNA, can induce ribosomal skipping during translation of the mRNA in a cell. Thus, two separate polypeptides may be produced from a single mRNA molecule. An amino acid sequence encoded by a 2A element is referred to as a “self-cleaving peptide.” 2A elements may be viral in origin. Exemplary 2A elements include T2A elements, P2A elements, E2A elements, and F2A elements.

As used herein, the term “P2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 19, or 21; (ii) encodes the amino acid sequence of SEQ ID NO: 18, or 20; or (iii) encodes the amino acid sequence of SEQ ID NO: 18, or 20, comprising 1, 2, or 3 amino acid modifications. In some embodiments, when positioned in a vector between a first polynucleotide sequence encoding a first protein and a second polynucleotide sequence encoding a second protein, the P2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the P2A element, e.g., such that the penultimate residue (e.g., glycine) becomes the C-terminal residue of the first protein and the ultimate residue (e.g., proline) becomes the N-terminal residue of the second protein. In some embodiments, the P2A element additionally comprises, at its 5′ end, a polynucleotide sequence that encodes a furin recognition site, e.g., RAKR (SEQ ID NO: 2). In some embodiments, the P2A element additionally comprises, at its 5′ end, a polynucleotide sequence that encodes a furin recognition site, e.g., RAKRSGSG (SEQ ID NO: 4), and the P2A element can be termed an “fP2A element.” In some embodiments, a fP2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 11; (ii) encodes the amino acid sequence of SEQ ID NO: 10; or (iii) encodes the amino acid sequence of SEQ ID NO: 10, comprising 1, 2, or 3 amino acid modifications. In some embodiments, the P2A element additionally comprises, at its 5′ end, a polynucleotide sequence that encodes a GSG (e.g., SEQ ID Nos: 20 and 21).

As used herein, the term “T2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 23, or 25; (ii) encodes the amino acid sequence of SEQ ID NO: 22, or 24; or (iii) encodes the amino acid sequence of SEQ ID NO: 22, or 24, comprising 1, 2, or 3 amino acid modifications. In some embodiments, when positioned in a vector between a first polynucleotide sequence encoding a first protein and a second polynucleotide sequence encoding a second protein, the T2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the T2A element, e.g., such that the penultimate residue (e.g., glycine) becomes the C-terminal residue of the first protein and the ultimate residue (e.g., proline) becomes the N-terminal residue of the second protein. In some embodiments, the T2A element additionally comprises, at its 5′ end, a polynucleotide sequence that encodes a furin recognition site, e.g., RAKR (SEQ ID NO: 2). In some embodiments, the T2A element additionally comprises, at its 5′ end, a polynucleotide sequence that encodes a furin recognition site, e.g., RAKRSGSG (SEQ ID NO: 4), and the T2A element can be termed an “fT2A element.” In some embodiments, an fT2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 13; (ii) encodes the amino acid sequence of SEQ ID NO: 12; or (iii) encodes the amino acid sequence of SEQ ID NO: 12, comprising 1, 2, or 3 amino acid modifications. In some embodiments, the T2A element additionally comprises, at its 5′ end, a polynucleotide sequence that encodes a GSG (e.g., SEQ ID Nos: 24 and 25).

As used herein, the term “F2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 27, or 29; (ii) encodes the amino acid sequence of SEQ ID NO: 26, or 28; or (iii) encodes the amino acid sequence of SEQ ID NO: 26, or 28, comprising 1, 2, or 3 amino acid modifications. In some embodiments, when positioned in a vector between a first polynucleotide sequence encoding a first protein and a second polynucleotide sequence encoding a second protein, the F2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the F2A element, e.g., such that the penultimate residue (e.g., glycine) becomes the C-terminal residue of the first protein and the ultimate residue (e.g., proline) becomes the N-terminal residue of the second protein. In some embodiments, the F2A element additionally comprises, at its 5′ end, a polynucleotide sequence that encodes a furin recognition site, e.g., RAKR (SEQ ID NO: 2). In some embodiments, the F2A element additionally comprises, at its 5′ end, a polynucleotide sequence that encodes a furin recognition site, e.g., RAKRSGSG (SEQ ID NO: 4), and the F2A element can be termed an “f2A element.” In some embodiments, a fF2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 15; (ii) encodes the amino acid sequence of SEQ ID NO: 14; or (iii) encodes the amino acid sequence of SEQ ID NO: 14, comprising 1, 2, or 3 amino acid modifications. In some embodiments, the F2A element additionally comprises, at its 5′ end, a polynucleotide sequence that encodes a GSG (e.g., SEQ ID Nos: 28 and 29).

As used herein, the term “E2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 31, or 33; (ii) encodes the amino acid sequence of SEQ ID NO: 30, or 32; or (iii) encodes the amino acid sequence of SEQ ID NO: 30, or 32, comprising 1, 2, or 3 amino acid modifications. In some embodiments, when positioned in a vector between a first polynucleotide sequence encoding a first protein and a second polynucleotide sequence encoding a second protein, the E2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the E2A element, e.g., such that the penultimate residue (e.g., glycine) becomes the C-terminal residue of the first protein and the ultimate residue (e.g., proline) becomes the N-terminal residue of the second protein. In some embodiments, the E2A element additionally comprises, at its 5′ end, a polynucleotide sequence that encodes a furin recognition site, e.g., RAKR (SEQ ID NO: 2). In some embodiments, the E2A element additionally comprises, at its 5′ end, a polynucleotide sequence that encodes a furin recognition site, e.g., RAKRSGSG (SEQ ID NO: 4), and the E2A element can be termed an “fE2A element.” In some embodiments, a fE2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 17; (ii) encodes the amino acid sequence of SEQ ID NO: 16; or (iii) encodes the amino acid sequence of SEQ ID NO: 16, comprising 1, 2, or 3 amino acid modifications. In some embodiments, the E2A element additionally comprises, at its 5′ end, a polynucleotide sequence that encodes a GSG (e.g., SEQ ID Nos: 32 and 33).

Examples of 2A elements comprising furin recognition sites at their N-terminal/5′ ends are found below in Table 2. The 2A sites themselves are broken out in Table 3.

TABLE 2
Polynucleotide sequences of exemplary furin-2A elements and
amino acid sequences of translations thereof.

| | | SEQ |
|---|---|---|
| Description | Sequence | ID NO: |
| Translation of Furin-P2A element | RAKRSGSGATNFSLLKQAGDVEENPGP | 10 |
| Furin-P2A element | CGGGCGAAACGCTCTGGAAGCGGAGCGACCAATTTCAGCCTGC | 11 |
| | TGAAGCAGGCGGGCGATGTGGAGGAGAACCCTGGCCCA | |
| Translation of Furin-T2A element | RAKRSGSGEGRGSLLTCGDVEENPGP | 12 |
| Furin-T2A element | CGGGCGAAACGCTCTGGAAGCGGAGAGGGCAGAGGAAGTCTTC | 13 |
| | TAACATGCGGTGACGTGGAGGAGAATCCCGGCCCT | |
| Translation of Furin-F2A element | RAKRSGSGVKQTLNFDLLKLAGDVESNPGP | 14 |
| Furin-F2A element | CGGGCGAAACGCTCTGGAAGCGGAGTGAAGCAGACCCTGAATT | 15 |
| | TCGACCTGCTGAAGCTGGCCGGGGACGTGGAGAGCAACCCTGG | |
| | CCCC | |
| Translation of Furin-E2A element | RAKRSGSGQCTNYALLKLAGDVESNPGP | 16 |
| Furin-E2A element | CGGGCGAAACGCTCTGGAAGCGGACAGTGTACTAATTATGCTC | 17 |
| | TCTTGAAATTGGCTGGAGATGTTGAGAGCAACCCAGGTCCC | |

TABLE 3
Amino acid and polynucleotide sequences of exemplary 2A elements.

| | | SEQ |
|---|---|---|
| Description | Amino Acid Sequence | ID NO: |
| P2A (exemplary amino acid | ATNFSLLKQAGDVEENPGP | 18 |
| sequence) | | |
| P2A (exemplary nucleotide | GCGACCAATTTCAGCCTGCTGAAGCAGGCGGGCGATGTGGAGG | 19 |
| sequence) | AGAACCCTGGCCCA | |
| P2A (with flanking residues) | GSGATNFSLLKQAGDVEENPGP | 20 |
| (exemplary amino acid sequence) | | |
| P2A (with flanking residues) | GGCTCCGGAGCGACCAATTTCAGCCTGCTGAAGCAGGCGGGCG | 21 |
| (exemplary nucleotide sequence) | ATGTGGAGGAGAACCCTGGCCCA | |
| T2A (exemplary amino acid | EGRGSLLTCGDVEENPGP | 22 |
| sequence) | | |
| T2A (exemplary nucleotide | GAGGGCAGAGGAAGTCTTCTAACATGCGGTGACGTGGAGGAGA | 23 |
| sequence) | ATCCCGGCCCT | |
| T2A (with flanking residues) | GSGEGRGSLLTCGDVEENPGP | 24 |
| (exemplary amino acid sequence) | | |
| T2A (with flanking residues) | GGCTCCGGAGAGGGCAGAGGAAGTCTTCTAACATGCGGTGACG | 25 |
| (exemplary nucleotide sequence) | TGGAGGAGAATCCCGGCCCT | |
| F2A (exemplary amino acid | VKQTLNFDLLKLAGDVESNPGP | 26 |
| sequence) | | |
| F2A (exemplary nucleotide | GTGAAGCAGACCCTGAATTTCGACCTGCTGAAGCTGGCCGGGG | 27 |
| sequence) | ACGTGGAGAGCAACCCTGGCCCC | |
| F2A (with flanking residues) | GSGVKQTLNFDLLKLAGDVESNPGP | 28 |
| (exemplary amino acid sequence) | | |
| F2A (with flanking residues) | GGCTCCGGAGTGAAGCAGACCCTGAATTTCGACCTGCTGAAGC | 29 |
| (exemplary nucleotide sequence) | TGGCCGGGGACGTGGAGAGCAACCCTGGCCCC | |
| E2A (exemplary amino acid | QCTNYALLKLAGDVESNPGP | 30 |
| sequence) | | |
| E2A (exemplary nucleotide | CAGTGTACTAATTATGCTCTCTTGAAATTGGCTGGAGATGTTG | 31 |
| sequence) | AGAGCAACCCAGGTCCC | |
| E2A (with flanking residues) | GSGQCTNYALLKLAGDVESNPGP | 32 |
| (exemplary amino acid sequence) | | |
| E2A (with flanking residues) | GGCTCCGGACAGTGTACTAATTATGCTCTCTTGAAATTGGCTG | 33 |
| (exemplary nucleotide sequence) | GAGATGTTGAGAGCAACCCAGGTCCC | |

As used herein, the terms “inverted terminal repeat,” “ITR,” “inverted repeat/direct repeat,” and “IR/DR” are used interchangeably and refer to a polynucleotide sequence, e.g., of about 230 nucleotides (e.g., 220, 221, 222, 223, 224, 225, 226, 227, 228, 229, 230, 231, 232, 233, 234, 235, 236, 237, 238, 239, or 240 nucleotides), flanking (e.g., with or without an intervening polynucleotide sequence) one end of an expression cassette (e.g., a polycistronic expression cassette) that can be cleaved by a transposase polypeptide when used in combination with a corresponding, e.g., reverse-complementary (e.g., perfectly or imperfectly reverse-complementary) polynucleotide sequence, e.g., of about 230 nucleotides (e.g., 220, 221, 222, 223, 224, 225, 226, 227, 228, 229, 230, 231, 232, 233, 234, 235, 236, 237, 238, 239, or 240 nucleotides), flanking (e.g., with or without an intervening polynucleotide sequence) the opposite end of the expression cassette (e.g., a polycistronic expression cassette) (e.g., as described in Cui et al., J. Mol. Biol. 2002; 318(5):1221-35, the contents of which are incorporated by reference in their entirety herein). In some embodiments, an ITR, e.g., an ITR of a DNA transposon (e.g., a Sleeping Beauty transposon, a piggyBac transposon, a TcBuster transposon, and a Tol2 transposon) contains two direct repeats (“DRs”), e.g., imperfect direct repeats, e.g., of about 30 nucleotides (e.g., 25, 26, 27, 28, 29, 30, 31, 32, 33, 34, or 35 nucleotides), located at each end of the ITR. The terms “ITR” and “DR,” when used in reference to a single- or double-stranded DNA vector, refer to the DNA sequence of the sense strand. A transposase polypeptide may recognize the sense strand and/or the antisense strand of DNA.

As used herein, the term “Left ITR,” when used in reference to a linear single- or double-stranded DNA vector, refers to the ITR positioned 5′ of the polycistronic expression cassette. As used herein, the term “Right ITR,” when used in reference to a linear single- or double-stranded DNA vector, refers to the ITR positioned 3′ of the polycistronic expression cassette. When a circular vector is used, the Left ITR is closer than the Right ITR to the 5′ end of the polycistronic expression cassette, and the Right ITR is closer than the Left ITR to the 3′ end of the polycistronic expression cassette.

As used herein, the term “operably linked” refers to a linkage of polynucleotide sequence elements or amino acid sequence elements in a functional relationship. For example, a polynucleotide sequence is operably linked when it is placed into a functional relationship with another polynucleotide sequence. In some embodiments, a transcription regulatory polynucleotide sequence e.g., a promoter, enhancer, or other expression control element is operably linked to a polynucleotide sequence that encodes a protein if it affects the transcription of the polynucleotide sequence that encodes the protein.

The term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “protein” and “polypeptide” are used interchangeably herein and refer to a polymer of amino acids connected by one or more peptide bonds. As used herein, “amino acid sequence” refers to the information describing the relative order and identity of amino acid residues which make up a polypeptide.

The term “functional variant” as used herein in reference to a protein or polypeptide refers to a protein that comprises at least one amino acid modification (e.g., a substitution, deletion, addition) compared to the amino acid sequence of a reference protein, that retains at least one particular function. In some embodiments, the reference protein is a wild type protein. For example, a functional variant of an IL-15 protein can refer to an IL-15 protein comprising an amino acid substitution compared to a wild type IL-15 protein that retains the ability to bind the IL-15 receptor α chain (IL-15Rα). Not all functions of the reference wild type protein need be retained by the functional variant of the protein. In some instances, one or more functions are selectively reduced or eliminated.

The term “functional fragment” as used herein in reference to a protein or polypeptide refers to a fragment of a reference protein that retains at least one particular function. For example, a functional fragment of an IL-15 protein can refer to a fragment of the protein that retains the ability to specifically bind IL-15Rα. Not all functions of the reference protein need be retained by a functional fragment of the protein. In some instances, one or more functions are selectively reduced or eliminated.

As used herein, the term “modification,” with reference to a polynucleotide sequence, refers to a polynucleotide sequence that comprises at least one substitution, alteration, inversion, addition, or deletion of nucleotide compared to a reference polynucleotide sequence. As used herein, the term “modification,” with reference to an amino acid sequence, refers to an amino acid sequence that comprises at least one substitution, alteration, inversion, addition, or deletion of an amino acid residue compared to a reference amino acid sequence.

As used herein, the term “derived from,” with reference to a polynucleotide sequence, refers to a polynucleotide sequence that has at least 85% sequence identity to a reference naturally occurring nucleic acid sequence from which it is derived. The term “derived from,” with reference to an amino acid sequence, refers to an amino acid sequence that has at least 85% sequence identity to a reference naturally occurring amino acid sequence from which it is derived. The term “derived from” as used herein does not denote any specific process or method for obtaining the polynucleotide or amino acid sequence. For example, the polynucleotide or amino acid sequence can be chemically synthesized.

As used herein, the term “linked to” refers to covalent or noncovalent binding between two molecules or moieties. The skilled worker will appreciate that when a first molecule or moiety is linked to a second molecule or moiety, the linkage need not be direct, but instead, can be via an intervening molecule or moiety.

As used herein, the term “cytokine” refers to a molecule that mediates and/or regulates a biological or cellular function or process (e.g., immunity, inflammation, and hematopoiesis). As used herein, cytokines include, but are not limited to, lymphokines, chemokines, monokines, and interleukins. The term cytokine as used herein also encompasses functional variants and functional variants of wild type cytokines.

As used herein, the term “marker protein” or “marker polypeptide” are used interchangeably and refer to a protein or polypeptide that can be expressed on the surface of a cell, which can be utilized to mark or deplete cells expressing the marker protein or polypeptide. In some embodiments, depletion of cells expressing the marker protein or polypeptide is performed through the administration of a molecule that specifically binds the marker protein or polypeptide (e.g., an antibody that mediates antibody dependent cellular cytotoxicity).

As used herein, the term “immune effector cell” refers to a cell that is involved in the promotion of an immune effector function. Examples of immune effector cells include, but are not limited to, T cells (e.g., alpha/beta T cells and gamma/delta T cells, CD4⁺ T cells, CD8⁺ T cells, natural killer T (NKT) cells), natural killer (NK) cells, B cells, mast cells, and myeloid-derived phagocytes.

As used herein, the term, “immune stem cell” refers to a cell that is pluripotent and can differentiate into one or more types of immune cells, including immune effector cells. Immune stem cells include, but are not limited to, bone marrow stem cells, hematopoietic stem cells, embryonic stem cells, induced pluripotent stem cells, umbilical blood stem cells, lymphocyte progenitor cells, stem cell memory T cells, and stem cell memory-like T cells. In certain embodiments, the immune stem cell is isolated and/or enriched from adult and fetal bone marrow, umbilical cord blood, or peripheral blood.

As used herein, the term “immune effector function” refers to a specialized function of an immune effector cell. The effector function of any given immune effector cell can be different. For example, an effector function of a CD8+ T cell is cytolytic activity, and an effector function of a CD4+ T cell is secretion of a cytokine.

### 5.2 T Cell Receptors

In one aspect, the instant disclosure provides TCRs that can be expressed via a polycistronic expression cassette of the present disclosure. In certain embodiments, the TCR comprises a T cell receptor (TCR) alpha chain comprising an alpha chain variable (Vα) region and an alpha chain constant (Cα) region and a TCR beta chain comprising a beta chain variable (Vβ) region and a beta chain constant (Cβ). The amino acid sequences of constant domains comprised in the TCRs disclosed herein are shown in Tables 4 and 5 below.

TABLE 4
Amino acid sequences of TCR Cα regions.

| | | SEQ |
|---|---|---|
| Description | Sequence | ID NO: |
| Cα (murine, | XIQNPEPAVYQLKDPRSQDSTLCLFTDFDSQINVPKTMESGTFITDKXVLDM | 40 |
| degenerate) | KAMDSKSNGAIAWSNQTSFTCQDIFKETNATYPSSDVPCDATLTEKSFETDM | |
| | NLNFQNLXVXXLRILLLKVAGFNLLMTLRLWSS | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cα (murine, | NNNATCCAGAATCCCGAGCCTGCGGTGTACCAGCTGAAGGACCCCCGCTCTC | 57 |
| degenerate) | AGGATAGCACACTGTGCCTGTTCACCGACTTTGATAGCCAGATCAACGTGCC | |
| (exemplary | TAAAACAATGGAGTCCGGCACCTTCATCACCGACAAGNNNGTGCTGGATATG | |
| nucleotide | AAAGCGATGGACTCCAAGTCTAACGGCGCGATCGCGTGGTCCAATCAGACAT | |
| sequence) | CTTTCACCTGCCAGGATATCTTCAAGGAGACAAACGCGACCTATCCTTCCTC | |
| | TGACGTGCCATGTGATGCGACACTGACCGAGAAGAGCTTCGAGACAGACATG | |
| | AACCTGAATTTTCAGAATCTGNNNGTCNNNNNNCTGAGAATCCTGCTGCTGA | |
| | AGGTGGCGGGCTTTAATCTGCTGATGACACTGCGGCTGTGGAGTTCC | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Val, | |
| | Leu, Ile, Pro, Phe, Met, or Trp; | |
| | NNN at positions 340-342 make up a codon that encodes Met, Ala, Val, | |
| | Leu, Ile, Pro, Phe, or Trp; | |
| | NNN at positions 343-345 make up a codon that encodes Gly, Ala, Val, | |
| | Leu, Ile, Pro, Phe, Met, or Trp | |
| Cα (murine, | NIQNPEPAVYQLKDPRSQDSTLCLFTDFDSQINVPKTMESGTFITDKCVLDM | 41 |
| cysteine- and | KAMDSKSNGAIAWSNQTSFTCQDIFKETNATYPSSDVPCDATLTEKSFETDM | |
| LIV-substituted) | NLNFQNLLVIVLRILLLKVAGFNLLMTLRLWSS | |
| Cα (murine, | AACATCCAGAATCCCGAGCCTGCGGTGTACCAGCTGAAGGACCCCCGCTCTC | 55 |
| cysteine- | AGGATAGCACACTGTGCCTGTTCACCGACTTTGATAGCCAGATCAACGTGCC | |
| and LIV- | TAAAACAATGGAGTCCGGCACCTTCATCACCGACAAGTGCGTGCTGGATATG | |
| substituted) | AAAGCGATGGACTCCAAGTCTAACGGCGCGATCGCGTGGTCCAATCAGACAT | |
| (exemplary | CTTTCACCTGCCAGGATATCTTCAAGGAGACAAACGCGACCTATCCTTCCTC | |
| nucleotide | TGACGTGCCATGTGATGCGACACTGACCGAGAAGAGCTTCGAGACAGACATG | |
| sequence) | AACCTGAATTTTCAGAATCTGCTGGTCATCGTGCTGAGAATCCTGCTGCTGA | |
| | AGGTGGCGGGCTTTAATCTGCTGATGACACTGCGGCTGTGGAGTTCC | |
| Cα (murine, LIV | NIQNPEPAVYQLKDPRSQDSTLCLFTDFDSQINVPKTMESGTFITDKTVLDM | 42 |
| substituted) | KAMDSKSNGAIAWSNQTSFTCQDIFKETNATYPSSDVPCDATLTEKSFETDM | |
| | NLNFQNLLVIVLRILLLKVAGFNLLMTLRLWSS | |
| Cα (murine, LIV | AACATCCAGAATCCCGAGCCTGCGGTGTACCAGCTGAAGGACCCCCGCTCTC | 58 |
| substituted) | AGGATAGCACACTGTGCCTGTTCACCGACTTTGATAGCCAGATCAACGTGCC | |
| (exemplary | TAAAACAATGGAGTCCGGCACCTTCATCACCGACAAGACCGTGCTGGATATG | |
| nucleotide | AAAGCGATGGACTCCAAGTCTAACGGCGCGATCGCGTGGTCCAATCAGACAT | |
| sequence) | CTTTCACCTGCCAGGATATCTTCAAGGAGACAAACGCGACCTATCCTTCCTC | |
| | TGACGTGCCATGTGATGCGACACTGACCGAGAAGAGCTTCGAGACAGACATG | |
| | AACCTGAATTTTCAGAATCTGCTGGTCATCGTGCTGAGAATCCTGCTGCTGA | |
| | AGGTGGCGGGCTTTAATCTGCTGATGACACTGCGGCTGTGGAGTTCC | |
| Cα (murine, | NIQNPEPAVYQLKDPRSQDSTLCLFTDFDSQINVPKTMESGTFITDKCVLDM | 43 |
| cysteine- | KAMDSKSNGAIAWSNQTSFTCQDIFKETNATYPSSDVPCDATLTEKSFETDM | |
| substituted) | NLNFQNLSVMGLRILLLKVAGFNLLMTLRLWSS | |
| Cα (murine, | NIQNPEPAVYQLKDPRSQDSTLCLFTDFDSQINVPKTMESGTFITDKTVLDM | 44 |
| wild type) | KAMDSKSNGAIAWSNQTSFTCQDIFKETNATYPSSDVPCDATLTEKSFETDM | |
| | NLNFQNLSVMGLRILLLKVAGFNLLMTLRLWSS | |
| Cα (human, | XIQNPDPAVYQLRDSKSSDKSVCLFTDFDSQTNVSQSKDSDVYITDKXVLDM | 45 |
| degenerate) | RSMDFKSNSAVAWSNKSDFACANAFNNSIIPEDTFFPSPESSCDVKLVEKSF | |
| | ETDTNLNFQNLXVXXFRILLLKVAGFNLLMTLRLWSS | |
| | X at position 1 is Asn, Asp, His, or Tyr | |
| | X at position 48 is Thr or Cys; | |
| | X at position 116 is Ser, Ala, Val, Leu, Ile, Pro, Phe, Met, or Trp; | |
| | X at position 118 is Met, Ala, Val, Leu, Ile, Pro, Phe, or Trp; | |
| | X at position 119 is Gly, Ala, Val, Leu, Ile, Pro, Phe, Met, or Trp | |
| Cα (human, | XIQNPDPAVYQLRDSKSSDKSVCLFTDFDSQTNVSQSKDSDVYITDKCVLDM | 46 |
| cysteine- | RSMDFKSNSAVAWSNKSDFACANAFNNSIIPEDTFFPSPESSCDVKLVEKSF | |
| and LIV- | ETDTNLNFQNLLVIVFRILLLKVAGFNLLMTLRLWSS | |
| substituted; | X at position 1 is Asn, Asp, His, or Tyr | |
| degenerate | | |
| at position 1) | | |
| Cα (human, LIV- | XIQNPDPAVYQLRDSKSSDKSVCLFTDFDSQTNVSQSKDSDVYITDKTVLDM | 47 |
| substituted; | RSMDFKSNSAVAWSNKSDFACANAFNNSIIPEDTFFPSPESSCDVKLVEKSF | |
| degenerate at | ETDTNLNFQNLLVIVFRILLLKVAGFNLLMTLRLWSS | |
| position 1) | X at position 1 is Asn, Asp, His, or Tyr | |
| Cα (human, | XIQNPDPAVYQLRDSKSSDKSVCLFTDFDSQTNVSQSKDSDVYITDKCVLDM | 48 |
| cysteine- | RSMDFKSNSAVAWSNKSDFACANAFNNSIIPEDTFFPSPESSCDVKLVEKSF | |
| substituted; | ETDTNLNFQNLSVIGFRILLLKVAGFNLLMTLRLWSS | |
| degenerate at | X at position 1 is Asn, Asp, His, or Tyr | |
| position 1) | | |
| Cα (human, | XIQNPDPAVYQLRDSKSSDKSVCLFTDFDSQTNVSQSKDSDVYITDKTVLDM | 49 |
| wild type; | RSMDFKSNSAVAWSNKSDFACANAFNNSIIPEDTFFPSPESSCDVKLVEKSF | |
| degenerate at | ETDTNLNFQNLSVIGFRILLLKVAGFNLLMTLRLWSS | |
| position 1) | X at position 1 is Asn, Asp, His, or Tyr | |

TABLE 5
Amino acid sequences of TCR Cβ regions.

| | | SEQ |
|---|---|---|
| Description | Sequence | ID NO: |
| Cβ (murine, | EDLRNVTPPKVSLFEPSKAEIANKQKATLVCLARGFFPDHVELSWWVNGKEV | 50 |
| degenerate) | HSGVXTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| | X at position 57 is Ser or Cys | |
| Cβ (murine, | GAGGACCTGAGGAACGTGACCCCACCTAAAGTGAGCCTGTTCGAGCCATCCA | 59 |
| degenerate) | AGGCGGAGATCGCGAATAAGCAGAAAGCGACCCTGGTGTGCCTGGCGAGGGG | |
| (exemplary nucleotide | CTTCTTTCCCGATCACGTGGAGCTGTCCTGGTGGGTGAACGGCAAAGAGGTG | |
| sequence) | CACTCTGGCGTGNNNACAGACCCTCAGGCGTACAAGGAGAGCAATTACTCCT | |
| | ATTGTCTGTCTAGCAGACTGAGGGTGAGCGCGACCTTTTGGCACAACCCCCG | |
| | GAATCACTTCCGCTGCCAGGTGCAGTTTCACGGCCTGTCCGAGGAGGATAAA | |
| | TGGCCTGAGGGCTCTCCAAAGCCCGTGACACAGAATATCAGCGCGGAGGCGT | |
| | GGGGAAGAGCGGACTGTGGCATTACAAGCGCGTCCTATCAGCAGGGCGTGCT | |
| | GTCCGCGACCATCCTGTACGAGATTCTGCTGGGCAAGGCGACACTGTATGCG | |
| | GTGCTGGTGTCCACCCTGGTGGTCATGGCGATGGTGAAGAGGAAAAACTCT | |
| | NNN at positions 169-171 make up a codon that encodes Ser or Cys | |
| Cβ (murine, cysteine- | EDLRNVTPPKVSLFEPSKAEIANKQKATLVCLARGFFPDHVELSWWVNGKEV | 51 |
| substituted) | HSGVCTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| Cβ (murine, cysteine- | GAGGACCTGAGGAACGTGACCCCACCTAAAGTGAGCCTGTTCGAGCCATCCA | 56 |
| substituted) | AGGCGGAGATCGCGAATAAGCAGAAAGCGACCCTGGTGTGCCTGGCGAGGGG | |
| (exemplary nucleotide | CTTCTTTCCCGATCACGTGGAGCTGTCCTGGTGGGTGAACGGCAAAGAGGTG | |
| sequence) | CACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCG | |
| | GAATCACTTCCGCTGCCAGGTGCAGTTTCACGGCCTGTCCGAGGAGGATAAA | |
| | TGGCCTGAGGGCTCTCCAAAGCCCGTGACACAGAATATCAGCGCGGAGGCGT | |
| | GGGGAAGAGCGGACTGTGGCATTACAAGCGCGTCCTATCAGCAGGGCGTGCT | |
| | GTCCGCGACCATCCTGTACGAGATTCTGCTGGGCAAGGCGACACTGTATGCG | |
| | GTGCTGGTGTCCACCCTGGTGGTCATGGCGATGGTGAAGAGGAAAAACTCT | |
| Cβ (murine, wild type) | EDLRNVTPPKVSLFEPSKAEIANKQKATLVCLARGFFPDHVELSWWVNGKEV | 52 |
| | HSGVSTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| Cβ (human, | EDLKNVFPPEVAVFEPSEAEISHTQKATLVCLATGFYPDHVELSWWVNGKEV | 53 |
| degenerate) | HSGVXTDPQPLKEQPALNDSRYCLSSRLRVSATFWQNPRNHFRCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |
| | X at position 57 is Ser or Cys | |
| Cβ (human, cysteine- | EDLKNVFPPEVAVFEPSEAEISHTQKATLVCLATGFYPDHVELSWWVNGKEV | 54 |
| substituted) | HSGVCTDPQPLKEQPALNDSRYCLSSRLRVSATFWQNPRNHERCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |
| Cβ (human, wild type) | EDLKNVFPPEVAVFEPSEAEISHTQKATLVCLATGFYPDHVELSWWVNGKEV | 60 |
| | HSGVSTDPQPLKEQPALNDSRYCLSSRLRVSATFWQNPRNHFRCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |

As used herein, “LIV-substituted” refers to a Cα sequence disclosed herein which, relative to SEQ ID NO: 40, comprises a leucine residue at position 112, an isoleucine residue at position 114, and a valine residue at position 115. See, for example, SEQ ID Nos: 41 and 42. In some embodiments, and independent of the LIV-substitutions a Cα sequence disclosed herein can comprise a cysteine at position 48, replacing the threonine residue. (Compare SEQ ID Nos: 40-44). In some embodiments, the Cβ sequence disclosed herein has a substitution of the serine at residue 57 with cysteine. This is shown in SEQ ID Nos: 50 and 51.

Tumor Protein p53 (also referred to as “p53”) acts as a tumor suppressor by, for example, regulating cell division. In some embodiments, wild type full-length p53 has the amino acid sequence of SEQ ID NO: 340, shown below.

(SEQ ID NO: 340)
MEEPQSDPSVEPPLSQETFSDLWKLLPENNVLSPLPSQAMDDLMLSPD
DIEQWFTEDPGPDEAPRMPEAAPPVAPAPAAPTPAAPAPAPSWPLSSS
VPSQKTYQGSYGFRLGFLHSGTAKSVTCTYSPALNKMFCQLAKTCPVQ
LWVDSTPPPGTRVRAMAIYKQSQHMTEVVRRCPHHERCSDSDGLAPPQ
HLIRVEGNLRVEYLDDRNTFRHSVVVPYEPPEVGSDCTTIHYNYMCNS
SCMGGMNRRPILTIITLEDSSGNLLGRNSFEVRVCACPGRDRRTEEEN
LRKKGEPHHELPPGSTKRALPNNTSSSPQPKKKPLDGEYFTLQIRGRE
RFEMFRELNEALELKDAQAGKEPGGSRAHSSHLKSKKGQSTSRHKKLM
EKTEGPDSD

Kirsten rat sarcoma viral oncogene homolog (KRAS), also referred to as GTPase Kras, V-Ki-Ras2 Kirsten rat sarcoma viral oncogene, or KRAS2, is a member of the small GTPase superfamily. There are two transcript variants of KRAS: KRAS variant A and KRAS variant B. Hereinafter, references to “KRAS” (mutated or unmutated) refer to both variant A and variant B, unless specified otherwise. In some embodiments, wild type KRAS variant A has the amino acid sequence of SEQ ID NO: 341 and wild type KRAS variant B has the amino acid sequence of SEQ ID NO: 342, both shown below.

(SEQ ID NO: 341)
MTEYKLVVVGAGGVGKSALTIQLIQNHFVDEYDPTIEDSYRKQVVIDG
ETCLLDILDTAGQEEYSAMRDQYMRTGEGFLCVFAINNTKSFEDIHHY
REQIKRVKDSEDVPMVLVGNKCDLPSRTVDTKQAQDLARSYGIPFIET
SAKTRQRVEDAFYTLVREIRQYRLKKISKEEKTPGCVKIKKCIIM
(SEQ ID NO: 342)
MTEYKLVVVGAGGVGKSALTIQLIQNHFVDEYDPTIEDSYRKQVVIDG
ETCLLDILDTAGQEEYSAMRDQYMRTGEGFLCVFAINNTKSFEDIHHY
REQIKRVKDSEDVPMVLVGNKCDLPSRTVDTKQAQDLARSYGIPFIET
SAKTRQGVDDAFYTLVREIRKHKEKMSKDGKKKKKKSKTKCVIM

EGFR (also referred to as ERBB1 or HER1) is a transmembrane glycoprotein that belongs to the receptor tyrosine kinase (RTK) super-family of cell surface receptors, which mediate cell signaling by extra-cellular growth factors. Examples of wild type (WT), unmutated human EGFR amino acid sequences include those disclosed in GenBank Accession Nos. NP_001 333826.1 (isoform e precursor), NP_001333827.1 (isoform f precursor), NP_001333828.1 (isoform g precursor), NP_001333829.1 (isoform h precursor), NP_001333870.1 (isoform i precursor), NP_005219.2 (isoform a precursor), NP_958439.1 (isoform b precursor), NP_958440.1 (isoform c precursor), and NP_95844l.1 (isoform d precursor). In some embodiments, wild type EGFR has the amino acid sequence of SEQ ID NO: 343

(SEQ ID NO: 343)
MRPSGTAGAALLALLAALCPASRALEEKKVCQGTSNKLTQLGTFEDHF
LSLQRMFNNCEVVLGNLEITYVQRNYDLSFLKTIQEVAGYVLIALNTV
ERIPLENLQIIRGNMYYENSYALAVLSNYDANKTGLKELPMRNLQEIL
HGAVRFSNNPALCNVESIQWRDIVSSDFLSNMSMDFQNHLGSCQKCDP
SCPNGSCWGAGEENCQKLTKIICAQQCSGRCRGKSPSDCCHNQCAAGC
TGPRESDCLVCRKFRDEATCKDTCPPLMLYNPTTYQMDVNPEGKYSFG
ATCVKKCPRNYVVTDHGSCVRACGADSYEMEEDGVRKCKKCEGPCRKV
CNGIGIGEFKDSLSINATNIKHFKNCTSISGDLHILPVAFRGDSFTHT
PPLDPQELDILKTVKEITGFLLIQAWPENRTDLHAFENLEIIRGRTKQ
HGQFSLAVVSLNITSLGLRSLKEISDGDVIISGNKNLCYANTINWKKL
FGTSGQKTKIISNRGENSCKATGQVCHALCSPEGCWGPEPRDCVSCRN
VSRGRECVDKCNLLEGEPREFVENSECIQCHPECLPQAMNITCTGRGP
DNCIQCAHYIDGPHCVKTCPAGVMGENNTLVWKYADAGHVCHLCHPNC
TYGCTGPGLEGCPTNGPKIPSIATGMVGALLLLLVVALGIGLFMRRRH
IVRKRTLRRLLQERELVEPLTPSGEAPNQALLRILKETEFKKIKVLGS
GAFGTVYKGLWIPEGEKVKIPVAIKELREATSPKANKEILDEAYVMAS
VDNPHVCRLLGICLTSTVQLITQLMPFGCLLDYVREHKDNIGSQYLLN
WCVQIAKGMNYLEDRRLVHRDLAARNVLVKTPQHVKITDFGLAKLLGA
EEKEYHAEGGKVPIKWMALESILHRIYTHQSDVWSYGVTVWELMTFGS
KPYDGIPASEISSILEKGERLPQPPICTIDVYMIMVKCWMIDADSRPK
FRELIIEFSKMARDPQRYLVIQGDERMHLPSPTDSNFYRALMDEEDMD
DVVDADEYLIPQQGFFSSPSTSRTPLLSSLSATSNNSTVACIDRNGLQ
SCPIKEDSFLQRYSSDPTGALTEDSIDDTFLPVPEYINQSVPKRPAGS
VQNPVYHNQPLNPAPSRDPHYQDPHSTAVGNPEYLNTVQPTCVNSTFD
SPAHWAQKGSHQISLDNPDYQQDFFPKEAKPNGIFKGSTAENAEYLRV
APQSSEFIGA

The amino acid sequences of exemplary TCRs are set forth in Table 6 herein.

TABLE 6A
Amino acid sequences of TCR001.

| | | SEQ |
|---|---|---|
| Description | Sequence | ID NO: |
| CDR1α | NYSPAY | 1001 |
| CDR2α | IRENEKE | 1002 |
| CDR3α | ALDIYPHDMR | 1003 |
| Vα without signal | QKIEQNSEALNIQEGKTATLTCNYTNYSPAYLQWYRQDPGRGPVFLLLIREN | 1004 |
| peptide (SignalP) | EKEKRKERLKVTFDTTLKQSLFHITASQPADSATYLCALDIYPHDMRFGAGT | |
| | RLTVKP | |
| Vα without signal | SQKIEQNSEALNIQEGKTATLTCNYTNYSPAYLQWYRQDPGRGPVFLLLIRE | 1005 |
| peptide (IMGT) | NEKEKRKERLKVTFDTTLKQSLFHITASQPADSATYLCALDIYPHDMRFGAG | |
| | TRLTVKP | |
| Vα | MXSFLGGVLLILWLQVDWVKSQKIEQNSEALNIQEGKTATLTCNYTNYSPAY | 1006 |
| | LQWYRQDPGRGPVFLLLIRENEKEKRKERLKVTFDTTLKQSLFHITASQPAD | 1007 |
| | SATYLCALDIYPHDMRFGAGTRLTVKP | |
| | (X = any amino acid) | |
| α chain with WT signal | MESFLGGVLLILWLQVDWVKSQKIEQNSEALNIQEGKTATLTCNYTNYSPAY | 1008 |
| peptide, Cα | LQWYRQDPGRGPVFLLLIRENEKEKRKERLKVTFDTTLKQSLFHITASQPAD | |
| (substituted) | SATYLCALDIYPHDMRFGAGTRLTVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α | MASFLGGVLLILWLQVDWVKSQKIEQNSEALNIQEGKTATLTCNYTNYSPAY | 1009 |
| chain with | LQWYRQDPGRGPVFLLLIRENEKEKRKERLKVTFDTTLKQSLFHITASQPAD | |
| alternative signal | SATYLCALDIYPHDMRFGAGTRLTVKPNIQNPEPAVYQLKDPRSQDSTLCLF | |
| peptide, Cα | TDFDSQINVPKTMESGTFITDKCVLDMKAMDSKSNGAIAWSNQTSFTCQDIF | |
| (substituted) | KETNATYPSSDVPCDATLTEKSFETDMNLNFQNLLVIVLRILLLKVAGFNLL | |
| | MTLRLWSS | |
| α chain with | MHSFLGGVLLILWLQVDWVKSQKIEQNSEALNIQEGKTATLTCNYTNYSPAY | 1010 |
| alternative signal | LQWYRQDPGRGPVFLLLIRENEKEKRKERLKVTFDTTLKQSLFHITASQPAD | |
| peptide, Cα | SATYLCALDIYPHDMRFGAGTRLTVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1ß | SGHAT | 2001 |
| CDR2ß | FQNNGV | 2002 |
| CDR3ß | ASSLDPGDTGELF | 2003 |
| Vß without signal | GVAQSPRYKIIEKRQSVAFWCNPISGHATLYWYQQILGQGPKLLIQFQNNGV | 2004 |
| peptide (SignalP) | VDDSQLPKDRFSAERLKGVDSTLKIQPAKLEDSAVYLCASSLDPGDTGELFF | |
| | GEGSRLTVL | |
| Vß without signal | EAGVAQSPRYKIIEKRQSVAFWCNPISGHATLYWYQQILGQGPKLLIQFQNN | 2005 |
| peptide (IMGT) | GVVDDSQLPKDRFSAERLKGVDSTLKIQPAKLEDSAVYLCASSLDPGDTGEL | |
| | FFGEGSRLTVL | |
| Vß | MXTRLLCWAALCLLGAELTEAGVAQSPRYKIIEKRQSVAFWCNPISGHATLY | 2006 |
| | WYQQILGQGPKLLIQFQNNGVVDDSQLPKDRFSAERLKGVDSTLKIQPAKLE | 2007 |
| | DSAVYLCASSLDPGDTGELFFGEGSRLTVL | |
| | (X = any amino acid) | |
| ß chain with WT signal | MGTRLLCWAALCLLGAELTEAGVAQSPRYKIIEKRQSVAFWCNPISGHATLY | 2008 |
| peptide, Cβ | WYQQILGQGPKLLIQFQNNGVVDDSQLPKDRFSAERLKGVDSTLKIQPAKLE | |
| (substituted) | DSAVYLCASSLDPGDTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain with alternative | MATRLLCWAALCLLGAELTEAGVAQSPRYKIIEKRQSVAFWCNPISGHATLY | 2009 |
| signal peptide, Cβ | WYQQILGQGPKLLIQFQNNGVVDDSQLPKDRFSAERLKGVDSTLKIQPAKLE | |
| (substituted) | DSAVYLCASSLDPGDTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain with alternative | MHTRLLCWAALCLLGAELTEAGVAQSPRYKIIEKRQSVAFWCNPISGHATLY | 2010 |
| signal peptide, Cβ | WYQQILGQGPKLLIQFQNNGVVDDSQLPKDRFSAERLKGVDSTLKIQPAKLE | |
| (substituted) | DSAVYLCASSLDPGDTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR001 interacts with and/or is specific for a peptide from the tumor protein p53 (p53). In some embodiments, the peptide is from a neoantigen of p53 and has the amino acid change R175H (in which position 175 of the p53 protein is mutated from Arg to His). In some embodiments, TCR001 interacts with and/or is specific for the neoantigen in the context of HLA-A*02:01, as described in International Publication No. WO 2019/067243, incorporated herein by reference in its entirety.

TABLE 6B
Amino acid sequences of TCR002.

| | | SEQ |
|---|---|---|
| Description | Sequence | ID NO: |
| CDR1α | DSASNY | 1011 |
| CDR2α | IRSNVGE | 1012 |
| CDR3α | AASKSAIMVVLQTSSSL | 1013 |
| Vα without signal | ENVEQHPSTLSVQEGDSAVIKCTYSDSASNYFPWYKQELGKGPQLIIDIRSN | 1014 |
| peptide (SignalP) | VGEKKDQRIAVTLNKTAKHFSLHITETQPEDSAVYFCAASKSAIMVVLQTSS | |
| | SLELALCLLSSQV | |
| Vα without signal | GENVEQHPSTLSVQEGDSAVIKCTYSDSASNYFPWYKQELGKGPQLIIDIRS | 1015 |
| peptide (IMGT) | NVGEKKDQRIAVTLNKTAKHFSLHITETQPEDSAVYFCAASKSAIMVVLQTS | |
| | SSLELALCLLSSQV | |
| Vα | MXSIRAVFIFLWLQLDLVNGENVEQHPSTLSVQEGDSAVIKCTYSDSASNYF | 1016 |
| | PWYKQELGKGPQLIIDIRSNVGEKKDQRIAVTLNKTAKHFSLHITETQPEDS | |
| | AVYFCAASKSAIMVVLQTSSSLELALCLLSSQV | 1017 |
| | (X = any amino acid) | |
| α chain with WT signal | MTSIRAVFIFLWLQLDLVNGENVEQHPSTLSVQEGDSAVIKCTYSDSASNYF | 1018 |
| peptide, Cα | PWYKQELGKGPQLIIDIRSNVGEKKDQRIAVTLNKTAKHFSLHITETQPEDS | |
| (substituted) | AVYFCAASKSAIMVVLQTSSSLELALCLLSSQVNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain with | MASIRAVFIFLWLQLDLVNGENVEQHPSTLSVQEGDSAVIKCTYSDSASNYF | 1019 |
| alternative signal | PWYKQELGKGPQLIIDIRSNVGEKKDQRIAVTLNKTAKHFSLHITETQPEDS | |
| peptide, Cα | AVYFCAASKSAIMVVLQTSSSLELALCLLSSQVNIQNPEPAVYQLKDPRSQD | |
| (substituted) | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain with | MHSIRAVFIFLWLQLDLVNGENVEQHPSTLSVQEGDSAVIKCTYSDSASNYF | 1020 |
| alternative signal | PWYKQELGKGPQLIIDIRSNVGEKKDQRIAVTLNKTAKHFSLHITETQPEDS | |
| peptide, Cα | AVYFCAASKSAIMVVLQTSSSLELALCLLSSQVNIQNPEPAVYQLKDPRSQD | |
| (substituted) | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| CDR1ß | MNHEY | 2011 |
| CDR2ß | SMNVEV | 2012 |
| CDR3ß | ASSIQQGADTQY | 2013 |
| Vß without signal | QVTQNPRYLITVTGKKLTVTCSQNMNHEYMSWYRQDPGLGLRQIYYSMNVEV | 2014 |
| peptide (SignalP) | TDKGDVPEGYKVSRKEKRNFPLILESPSPNQTSLYFCASSIQQGADTQYFGP | |
| | GTRLTVL | |
| Vß without signal | EAQVTQNPRYLITVTGKKLTVTCSQNMNHEYMSWYRQDPGLGLRQIYYSMNV | 2015 |
| peptide (IMGT) | EVTDKGDVPEGYKVSRKEKRNFPLILESPSPNQTSLYFCASSIQQGADTQYF | |
| | GPGTRLTVL | |
| Vß | MXPQLLGYVVLCLLGAGPLEAQVTQNPRYLITVTGKKLTVTCSQNMNHEYMS | 2016 |
| | WYRQDPGLGLRQIYYSMNVEVTDKGDVPEGYKVSRKEKRNFPLILESPSPNQ | |
| | TSLYFCASSIQQGADTQYFGPGTRLTVL | 2017 |
| | (X = any amino acid) | |
| ß chain with WT signal | MGPQLLGYVVLCLLGAGPLEAQVTQNPRYLITVTGKKLTVTCSQNMNHEYMS | 2018 |
| peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain with alternative | MAPQLLGYVVLCLLGAGPLEAQVTQNPRYLITVTGKKLTVTCSQNMNHEYMS | 2019 |
| signal peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain with alternative | MHPQLLGYVVLCLLGAGPLEAQVTQNPRYLITVTGKKLTVTCSQNMNHEYMS | 2020 |
| signal peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR002 interacts with and/or is specific for a peptide from p53. In some embodiments, the peptide is from a neoantigen of p53. In some embodiments, the neoantigen has the amino acid change R175H relative to the wild type p53 sequence. In some embodiments, TCR002 interacts with the neoantigen in the context of HLA-A*02:01, as described in International Publication No. WO 2019/067243, incorporated herein by reference in its entirety.

TABLE 6C
Amino acid sequences of TCR003.

| | | SEQ |
|---|---|---|
| Description | Sequence | ID NO: |
| CDR1α | NSAFQY | 1021 |
| CDR2α | TYSSGN | 1022 |
| CDR3α | AMSGLKEDSSYKLI | 1023 |
| Vα w/o signal peptide | QQKEVEQDPGPLSVPEGAIVSLNCTYSNSAFQYFMWYRQYSRKGPELLMYTY | 1024 |
| (SignalP) | SSGNKEDGRFTAQVDKSSKYISLFIRDSQPSDSATYLCAMSGLKEDSSYKLI | |
| | FGSGTRLLVRP | |
| Vα w/o signal peptide | QKEVEQDPGPLSVPEGAIVSLNCTYSNSAFQYFMWYRQYSRKGPELLMYTYS | 1025 |
| (IMGT) | SGNKEDGRFTAQVDKSSKYISLFIRDSQPSDSATYLCAMSGLKEDSSYKLIF | |
| | GSGTRLLVRP | |
| Vα | MXKSLRVLLVILWLQLSWVWSQQKEVEQDPGPLSVPEGAIVSLNCTYSNSAF | 1026 |
| | QYFMWYRQYSRKGPELLMYTYSSGNKEDGRFTAQVDKSSKYISLFIRDSQPS | 1027 |
| | DSATYLCAMSGLKEDSSYKLIFGSGTRLLVRP | |
| | (X = any amino acid) | |
| α chain w/WT signal | MMKSLRVLLVILWLQLSWVWSQQKEVEQDPGPLSVPEGAIVSLNCTYSNSAF | 1028 |
| peptide, Co | QYFMWYRQYSRKGPELLMYTYSSGNKEDGRFTAQVDKSSKYISLFIRDSQPS | |
| (substituted) | DSATYLCAMSGLKEDSSYKLIFGSGTRLLVRPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain w/alternative | MAKSLRVLLVILWLQLSWVWSQQKEVEQDPGPLSVPEGAIVSINCTYSNSAF | 1029 |
| signal peptide, Cα | QYFMWYRQYSRKGPELLMYTYSSGNKEDGRFTAQVDKSSKYISLFIRDSQPS | |
| (substituted) | DSATYLCAMSGLKEDSSYKLIFGSGTRLLVRPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| a chain w/alternative | MHKSLRVLLVILWLQLSWVWSQQKEVEQDPGPLSVPEGAIVSLNCTYSNSAF | 1030 |
| signal peptide, Cα | QYFMWYRQYSRKGPELLMYTYSSGNKEDGRFTAQVDKSSKYISLFIRDSQPS | |
| (substituted) | DSATYLCAMSGLKEDSSYKLIFGSGTRLLVRPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1ß | MNHEY | 2021 |
| CDR2ß | SMNVEV | 2022 |
| CDR3ß | ASSIQQGADTQY | 2023 |
| Vß w/o signal peptide | QVTQNPRYLITVTGKKLTVTCSQNMNHEYMSWYRQDPGLGLRQIYYSMNVEV | 2024 |
| (SignalP) | TDKGDVPEGYKVSRKEKRNFPLILESPSPNQTSLYFCASSIQQGADTQYFGP | |
| | GTRLTVL | |
| Vß w/o signal peptide | EAQVTQNPRYLITVTGKKLTVTCSQNMNHEYMSWYRQDPGLGLRQIYYSMNV | 2025 |
| (IMGT) | EVTDKGDVPEGYKVSRKEKRNFPLILESPSPNQTSLYFCASSIQQGADTQYF | |
| | GPGTRLTVL | |
| Vß | MXPQLLGYVVLCLLGAGPLEAQVTQNPRYLITVTGKKLTVTCSQNMNHEYMS | 2026 |
| | WYRQDPGLGLRQIYYSMNVEVTDKGDVPEGYKVSRKEKRNFPLILESPSPNQ | 2027 |
| | TSLYFCASSIQQGADTQYFGPGTRLTVL | |
| | (X = any amino acid) | |
| ß chain w/WT signal | MGPQLLGYVVLCLLGAGPLEAQVTQNPRYLITVTGKKLTVTCSQNMNHEYMS | 2028 |
| peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MAPQLLGYVVLCLLGAGPLEAQVTQNPRYLITVTGKKLTVTCSQNMNHEYMS | 2029 |
| signal peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MHPQLLGYVVLCLLGAGPLEAQVTQNPRYLITVTGKKLTVTCSQNMNHEYMS | 2030 |
| signal peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR003 interacts with and/or is specific for a peptide from p53. In some embodiments, the peptide is from a neoantigen of p53. In some embodiments, the neoantigen has the amino acid change R175H relative to the wild type p53 sequence. In some embodiments, TCR003 interacts with the neoantigen in the context of HLA-A*02:01, as described in International Publication No. WO 2020/264269, incorporated herein by reference in its entirety.

TABLE 6D
Amino acid sequences of TCR004.

| | | SEQ |
|---|---|---|
| Description | Sequence | ID NO: |
| CDR1α | NSASQS | 1031 |
| CDR2α | VYSSGN | 1032 |
| CDR3α | VVQPGGYQKVT | 1033 |
| Vα w/o signal peptide | QRKEVEQDPGPFNVPEGATVAFNCTYSNSASQSFFWYRQDCRKEPKLLMSVY | 1034 |
| (SignalP) | SSGNEDGRFTAQLNRASQYISLLIRDSKLSDSATYLCVVQPGGYQKVTFGTG | |
| | TKLQVIP | |
| Vα w/o signal peptide | RKEVEQDPGPFNVPEGATVAFNCTYSNSASQSFFWYRQDCRKEPKLLMSVYS | 1035 |
| (IMGT) | SGNEDGRFTAQLNRASQYISLLIRDSKLSDSATYLCVVQPGGYQKVTFGTGT | |
| | KLQVIP | |
| Vα | MXSLRVLLVILWLQLSWVWSQRKEVEQDPGPFNVPEGATVAFNCTYSNSASQ | 1036 |
| | SFFWYRQDCRKEPKLLMSVYSSGNEDGRFTAQLNRASQYISLLIRDSKLSDS | 1037 |
| | ATYLCVVQPGGYQKVTFGTGTKLQVIP | |
| | (X = any amino acid) | |
| α chain w/WT signal | MISLRVLLVILWLQLSWVWSQRKEVEQDPGPFNVPEGATVAFNCTYSNSASQ | 1038 |
| peptide, Cα | SFFWYRQDCRKEPKLLMSVYSSGNEDGRFTAQLNRASQYISLLIRDSKLSDS | |
| (substituted) | ATYLCVVQPGGYQKVTFGTGTKLQVIPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MASLRVLLVILWLQLSWVWSQRKEVEQDPGPFNVPEGATVAFNCTYSNSASQ | 1039 |
| signal peptide, Cα | SFFWYRQDCRKEPKLLMSVYSSGNEDGRFTAQLNRASQYISLLIRDSKLSDS | |
| (substituted) | ATYLCVVQPGGYQKVTFGTGTKLQVIPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MHSLRVLLVILWLQLSWVWSQRKEVEQDPGPFNVPEGATVAFNCTYSNSASQ | 1040 |
| signal peptide, Cα | SFFWYRQDCRKEPKLLMSVYSSGNEDGRFTAQLNRASQYISLLIRDSKLSDS | |
| (substituted) | ATYLCVVQPGGYQKVTFGTGTKLQVIPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1ß | MNHNS | 2031 |
| CDR2ß | SASEGT | 2032 |
| CDR3ß | ASSEGLWQVGDEQY | 2033 |
| Vß w/o signal peptide | GVTQTPKFQVLKTGQSMTLQCAQDMNHNSMYWYRQDPGMGLRLIYYSASEGT | 2034 |
| (SignalP) | TDKGEVPNGYNVSRLNKREFSLRLESAAPSQTSVYFCASSEGLWQVGDEQYF | |
| | GPGTRLTVT | |
| Vß w/o signal peptide | NAGVTQTPKFQVLKTGQSMTLQCAQDMNHNSMYWYRQDPGMGLRLIYYSASE | 2035 |
| (IMGT) | GTTDKGEVPNGYNVSRLNKREFSLRLESAAPSQTSVYFCASSEGLWQVGDEQ | |
| | YFGPGTRLTVT | |
| Vß | MXIGLLCCVAFSLLWASPVNAGVTQTPKFQVLKTGQSMTLQCAQDMNHNSMY | 2036 |
| | WYRQDPGMGLRLIYYSASEGTTDKGEVPNGYNVSRLNKREFSLRLESAAPSQ | 2037 |
| | TSVYFCASSEGLWQVGDEQYFGPGTRLTVT | |
| | (X = any amino acid) | |
| ß chain w/WT signal | MSIGLLCCVAFSLLWASPVNAGVTQTPKFQVLKTGQSMTLQCAQDMNHNSMY | 2038 |
| peptide, Cβ | WYRQDPGMGLRLIYYSASEGTTDKGEVPNGYNVSRLNKREFSLRLESAAPSQ | |
| (substituted) | TSVYFCASSEGLWQVGDEQYFGPGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MAIGLLCCVAFSLLWASPVNAGVTQTPKFQVLKTGQSMTLQCAQDMNHNSMY | 2039 |
| signal peptide, Cβ | WYRQDPGMGLRLIYYSASEGTTDKGEVPNGYNVSRLNKREFSLRLESAAPSQ | |
| (substituted) | TSVYFCASSEGLWQVGDEQYFGPGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MHIGLLCCVAFSLLWASPVNAGVTQTPKFQVLKTGQSMTLQCAQDMNHNSMY | 2040 |
| signal peptide, Cβ | WYRQDPGMGLRLIYYSASEGTTDKGEVPNGYNVSRLNKREFSLRLESAAPSQ | |
| (substituted) | TSVYFCASSEGLWQVGDEQYFGPGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR004 interacts with and/or is specific for p53. In some embodiments, the peptide is from a neoantigen of p53. In some embodiments, the neoantigen has the amino acid change R175H relative to the wild type p53 sequence. In some embodiments, TCR004 interacts with the neoantigen in the context of HLA-A*02:01, as described in

TABLE 6E
Amino acid sequences of TCR005.

| | | SEQ |
|---|---|---|
| Description | Sequence | ID NO: |
| CDR1α | TSENNYY | 1041 |
| CDR2α | QEAYKQQN | 1042 |
| CDR3α | AFMGYSGAGSYQLT | 1043 |
| Vα w/o signal peptide | QTVTQSQPEMSVQEAETVTLSCTYDTSENNYYLFWYKQPPSRQMILVIRQEA | 1044 |
| (SignalP) | YKQQNATENRFSVNFQKAAKSFSLKISDSQLGDTAMYFCAFMGYSGAGSYQL | |
| | TFGKGTKLSVIP | |
| Vα w/o signal peptide | AQTVTQSQPEMSVQEAETVTLSCTYDTSENNYYLFWYKQPPSRQMILVIRQE | 1045 |
| (IMGT) | AYKQQNATENRFSVNFQKAAKSFSLKISDSQLGDTAMYFCAFMGYSGAGSYQ | |
| | LTFGKGTKLSVIP | |
| Vα | MXRVSLLWAVVVSTCLESGMAQTVTQSQPEMSVQEAETVTLSCTYDTSENNY | 1046 |
| | YLFWYKQPPSRQMILVIRQEAYKQQNATENRFSVNFQKAAKSFSLKISDSQL | 1047 |
| | GDTAMYFCAFMGYSGAGSYQLTFGKGTKLSVIP | |
| | (X = any amino acid) | |
| α chain w/WT signal | MTRVSLLWAVVVSTCLESGMAQTVTQSQPEMSVQEAETVTLSCTYDTSENNY | 1048 |
| peptide, Cα | YLFWYKQPPSRQMILVIRQEAYKQQNATENRFSVNFQKAAKSFSLKISDSQL | |
| (substituted) | GDTAMYFCAFMGYSGAGSYQLTFGKGTKLSVIPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MARVSLLWAVVVSTCLESGMAQTVTQSQPEMSVQEAETVTLSCTYDTSENNY | 1049 |
| signal peptide, Cα | YLFWYKQPPSRQMILVIRQEAYKQQNATENRFSVNFQKAAKSFSLKISDSQL | |
| (substituted) | GDTAMYFCAFMGYSGAGSYQLTFGKGTKLSVIPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MHRVSLLWAVVVSTCLESGMAQTVTQSQPEMSVQEAETVTLSCTYDTSENNY | 1050 |
| signal peptide, Cα | YLFWYKQPPSRQMILVIRQEAYKQQNATENRFSVNFQKAAKSFSLKISDSQL | |
| (substituted) | GDTAMYFCAFMGYSGAGSYQLTFGKGTKLSVIPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| CDR1ß | ENHRY | 2041 |
| CDR2ß | SYGVKD | 2042 |
| CDR3ß | AISELVTGDSPLH | 2043 |
| Vß w/o signal peptide | GITQSPRHKVTETGTPVTLRCHQTENHRYMYWYRQDPGHGLRLIHYSYGVKD | 2044 |
| (SignalP) | TDKGEVSDGYSVSRSKTEDFLLTLESATSSQTSVYFCAISELVTGDSPLHFG | |
| | NGTRLTVT | |
| Vß w/o signal peptide | DAGITQSPRHKVTETGTPVTLRCHQTENHRYMYWYRQDPGHGLRLIHYSYGV | 2045 |
| (IMGT) | KDTDKGEVSDGYSVSRSKTEDFLLTLESATSSQTSVYFCAISELVTGDSPLH | |
| | FGNGTRLTVT | |
| Vß | MXTRLFFYVALCLLWTGHMDAGITQSPRHKVTETGTPVTLRCHQTENHRYMY | 2046 |
| | WYRQDPGHGLRLIHYSYGVKDTDKGEVSDGYSVSRSKTEDELLTLESATSSQ | 2047 |
| | TSVYFCAISELVTGDSPLHFGNGTRLTVT | |
| | (X = any amino acid) | |
| ß chain w/WT signal | MGTRLFFYVALCLLWTGHMDAGITQSPRHKVTETGTPVTLRCHQTENHRYMY | 2048 |
| peptide, Cβ | WYRQDPGHGLRLIHYSYGVKDTDKGEVSDGYSVSRSKTEDELLTLESATSSQ | |
| (substituted) | TSVYFCAISELVTGDSPLHFGNGTRLTVTEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MATRLFFYVALCLLWTGHMDAGITQSPRHKVTETGTPVTLRCHQTENHRYMY | 2049 |
| signal peptide, Cβ | WYRQDPGHGLRLIHYSYGVKDTDKGEVSDGYSVSRSKTEDFLLTLESATSSQ | |
| (substituted) | TSVYFCAISELVTGDSPLHFGNGTRLTVTEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MHTRLFFYVALCLLWTGHMDAGITQSPRHKVTETGTPVTLRCHQTENHRYMY | 2050 |
| signal peptide, Cβ | WYRQDPGHGLRLIHYSYGVKDTDKGEVSDGYSVSRSKTEDFLLTLESATSSQ | |
| (substituted) | TSVYFCAISELVTGDSPLHFGNGTRLTVTEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR005 interacts with and/or is specific for p53. In some embodiments, the peptide is from a neoantigen of p53. In some embodiments, the neoantigen has the amino acid change R175H relative to the wild type p53 sequence. In some embodiments, TCR005 interacts with the neoantigen in the context of HLA-A*02:01, as described in

TABLE 6F
Amino acid sequences of TCR006.

| | | SEQ |
|---|---|---|
| Description | Sequence | ID NO: |
| CDR1α | TISGNEY | 1051 |
| CDRαa | GLKNN | 1052 |
| CDR3α | IVRGSPGAGGTSYGKLT | 1053 |
| Vα w/o signal peptide | KTTQPPSMDCAEGRAANLPCNHSTISGNEYVYWYRQIHSQGPQYIIHGLKNN | 1054 |
| (SignalP) | ETNEMASLIITEDRKSSTLILPHATLRDTAVYYCIVRGSPGAGGTSYGKLTF | |
| | GQGTILTVHP | |
| Vα w/o signal peptide | DAKTTQPPSMDCAEGRAANLPCNHSTISGNEYVYWYRQIHSQGPQYIIHGLK | 1055 |
| (IMGT) | NNETNEMASLIITEDRKSSTLILPHATLRDTAVYYCIVRGSPGAGGTSYGKL | |
| | TFGQGTILTVHP | |
| Vα | MXLVARVTVFLTFGTIIDAKTTQPPSMDCAEGRAANLPCNHSTISGNEYVYW | 1056 |
| | YRQIHSQGPQYIIHGLKNNETNEMASLIITEDRKSSTLILPHATLRDTAVYY | 1057 |
| | CIVRGSPGAGGTSYGKLTFGQGTILTVHP | |
| | (X = any amino acid) | |
| α chain w/WT signal | MRLVARVTVFLTFGTIIDAKTTQPPSMDCAEGRAANLPCNHSTISGNEYVYW | 1058 |
| peptide, Cα | YRQIHSQGPQYIIHGLKNNETNEMASLIITEDRKSSTLILPHATLRDTAVYY | |
| (substituted) | CIVRGSPGAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MALVARVTVFLTFGTIIDAKTTQPPSMDCAEGRAANLPCNHSTISGNEYVYW | 1059 |
| signal peptide, Cα | YRQIHSQGPQYIIHGLKNNETNEMASLIITEDRKSSTLILPHATLRDTAVYY | |
| (substituted) | CIVRGSPGAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MHLVARVTVFLTFGTIIDAKTTQPPSMDCAEGRAANLPCNHSTISGNEYVYW | 1060 |
| signal peptide, Cα | YRQIHSQGPQYIIHGLKNNETNEMASLIITEDRKSSTLILPHATLRDTAVYY | |
| (substituted) | CIVRGSPGAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1ß | LNHDA | 2051 |
| CDR2ß | SQIVND | 2052 |
| CDR3ß | ASSIRTEAF | 2053 |
| Vß w/o signal peptide | GITQSPKYLFRKEGQNVTLSCEQNLNHDAMYWYRQDPGQGLRLIYYSQIVND | 2054 |
| (SignalP) | FQKGDIAEGYSVSREKKESFPLTVTSAQKNPTAFYLCASSIRTEAFFGQGTR | |
| | LTVV | |
| Vß w/o signal peptide | DGGITQSPKYLFRKEGQNVTLSCEQNLNHDAMYWYRQDPGQGLRLIYYSQIV | 2055 |
| (IMGT) | NDFQKGDIAEGYSVSREKKESFPLTVTSAQKNPTAFYLCASSIRTEAFFGQG | |
| | TRLTVV | |
| Vß | MXNQVLCCVVLCFLGANTVDGGITQSPKYLFRKEGQNVTLSCEQNLNHDAMY | 2056 |
| | WYRQDPGQGLRLIYYSQIVNDFQKGDIAEGYSVSREKKESFPLTVTSAQKNP | 2057 |
| | TAFYLCASSIRTEAFFGQGTRLTVV | |
| | (X = any amino acid) | |
| ß chain w/WT signal | MSNQVLCCVVLCFLGANTVDGGITQSPKYLFRKEGQNVTLSCEQNLNHDAMY | 2058 |
| peptide, Cβ | WYRQDPGQGLRLIYYSQIVNDFQKGDIAEGYSVSREKKESFPLTVTSAQKNP | |
| (substituted) | TAFYLCASSIRTEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| ß chain w/alternative | MANQVLCCVVLCFLGANTVDGGITQSPKYLFRKEGQNVTLSCEQNLNHDAMY | 2059 |
| signal peptide, Cβ | WYRQDPGQGLRLIYYSQIVNDFQKGDIAEGYSVSREKKESFPLTVTSAQKNP | |
| (substituted) | TAFYLCASSIRTEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| ß chain w/alternative | MHNQVLCCVVLCFLGANTVDGGITQSPKYLFRKEGQNVTLSCEQNLNHDAMY | 2060 |
| signal peptide, Cβ | WYRQDPGQGLRLIYYSQIVNDFQKGDIAEGYSVSREKKESFPLTVTSAQKNP | |
| (substituted) | TAFYLCASSIRTEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR006 interacts with and/or is specific for p53. In some embodiments, the peptide is from a neoantigen of p53. In some embodiments, the neoantigen has the amino acid change R175H relative to the wild type p53 sequence. In some embodiments, TCR006 interacts with the neoantigen in the context of HLA-DRB1*13:01, as described in International Publication No. WO 2020/264269, incorporated herein by reference in its entirety.

TABLE 6G
Amino acid sequences of TCR007.

| | | SEQ |
|---|---|---|
| Description | Sequence | ID NO: |
| CDR1α | TTSDR | 1061 |
| CDR2α | LLSNGAV | 1062 |
| CDR3α | AVAHMDSNYQLI | 1063 |
| Vα w/o signal peptide | ELKVEQNPLFLSMQEGKNYTIYCNYSTTSDRLYWYRQDPGKSLESLFVLLSN | 1064 |
| (SignalP) | GAVKQEGRLMASLDTKARLSTLHITAAVHDLSATYFCAVAHMDSNYQLIWGA | |
| | GTKLIIKP | |
| Vα w/o signal peptide | ELKVEQNPLFLSMQEGKNYTIYCNYSTTSDRLYWYRQDPGKSLESLFVLLSN | 1065 |
| (IMGT) | GAVKQEGRLMASLDTKARLSTLHITAAVHDLSATYFCAVAHMDSNYQLIWGA | |
| | GTKLIIKP | |
| Vα | MXKLLAMILWLQLDRLSGELKVEQNPLFLSMQEGKNYTIYCNYSTTSDRLYW | 1066 |
| | YRQDPGKSLESLFVLLSNGAVKQEGRLMASLDTKARLSTLHITAAVHDLSAT | 1067 |
| | YFCAVAHMDSNYQLIWGAGTKLIIKP | |
| | (X = any amino acid) | |
| α chain w/WT signal | MKKLLAMILWLQLDRLSGELKVEQNPLFLSMQEGKNYTIYCNYSTTSDRLYW | 1068 |
| peptide, Cα | YRQDPGKSLESLFVLLSNGAVKQEGRLMASLDTKARLSTLHITAAVHDLSAT | |
| (substituted) | YFCAVAHMDSNYQLIWGAGTKLIIKPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MAKLLAMILWLQLDRLSGELKVEQNPLFLSMQEGKNYTIYCNYSTTSDRLYW | 1069 |
| signal peptide, Cα | YRQDPGKSLESLFVLLSNGAVKQEGRLMASLDTKARLSTLHITAAVHDLSAT | |
| (substituted) | YFCAVAHMDSNYQLIWGAGTKLIIKPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MHKLLAMILWLQLDRLSGELKVEQNPLFLSMQEGKNYTIYCNYSTTSDRLYW | 1070 |
| signal peptide, Cα | YRQDPGKSLESLFVLLSNGAVKQEGRLMASLDTKARLSTLHITAAVHDLSAT | |
| (substituted) | YFCAVAHMDSNYQLIWGAGTKLIIKPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1ß | MNHEY | 2061 |
| CDR2ß | SVGEGT | 2062 |
| CDR3ß | ASSYAGLAAPREQF | 2063 |
| Vß w/o signal peptide | GVTQTPKFRVLKTGQSMTLLCAQDMNHEYMYWYRQDPGMGLRLIHYSVGEGT | 2064 |
| (SignalP) | TAKGEVPDGYNVSRLKKQNFLLGLESAAPSQTSVYFCASSYAGLAAPREQFF | |
| | GPGTRLTVL | |
| Vß w/o signal peptide | NAGVTQTPKFRVLKTGQSMTLLCAQDMNHEYMYWYRQDPGMGLRLIHYSVGE | 2065 |
| (IMGT) | GTTAKGEVPDGYNVSRLKKQNFLLGLESAAPSQTSVYFCASSYAGLAAPREQ | |
| | FFGPGTRLTVL | |
| Vß | MXLGLLCCGAFSLLWAGPVNAGVTQTPKFRVLKTGQSMTLLCAQDMNHEYMY | 2066 |
| | WYRQDPGMGLRLIHYSVGEGTTAKGEVPDGYNVSRLKKQNFLLGLESAAPSQ | 2067 |
| | TSVYFCASSYAGLAAPREQFFGPGTRLTVL | |
| | (X = any amino acid) | |
| ß chain w/WT signal | MSLGLLCCGAFSLLWAGPVNAGVTQTPKFRVLKTGQSMTLLCAQDMNHEYMY | 2068 |
| peptide, Cβ | WYRQDPGMGLRLIHYSVGEGTTAKGEVPDGYNVSRLKKQNFLLGLESAAPSQ | |
| (substituted) | TSVYFCASSYAGLAAPREQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MALGLLCCGAFSLLWAGPVNAGVTQTPKFRVLKTGQSMTLLCAQDMNHEYMY | 2069 |
| signal peptide, Cβ | WYRQDPGMGLRLIHYSVGEGTTAKGEVPDGYNVSRLKKQNFLLGLESAAPSQ | |
| (substituted) | TSVYFCASSYAGLAAPREQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MHLGLLCCGAFSLLWAGPVNAGVTQTPKFRVLKTGQSMTLLCAQDMNHEYMY | 2070 |
| signal peptide, Cβ | WYRQDPGMGLRLIHYSVGEGTTAKGEVPDGYNVSRLKKQNFLLGLESAAPSQ | |
| (substituted) | TSVYFCASSYAGLAAPREQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR007 interacts with and/or is specific for p53. In some embodiments, the peptide is from a neoantigen of p53. In some embodiments, the neoantigen has the amino acid change R175H relative to the wild type p53 sequence. In some embodiments, TCR007 interacts with the neoantigen in the context of HLA-DRB1*13:01, as described in International Publication No. WO 2020/264269, incorporated herein by reference in its entirety.

TABLE 6H
Amino acid sequences of TCR008.

| | | SEQ |
|---|---|---|
| Description | Sequence | ID NO: |
| CDR1α | TISGNEY | 1071 |
| CDR2α | GLKNN | 1072 |
| CDR3α | IVRARANAGGTSYGKLT | 1073 |
| Vα w/o signal peptide | KTTQPPSMDCAEGRAANLPCNHSTISGNEYVYWYRQIHSQGPQYIIHGLKNN | 1074 |
| (SignalP) | ETNEMASLIITEDRKSSTLILPHATLRDTAVYYCIVRARANAGGTSYGKLTF | |
| | GQGTILTVHP | |
| Vα w/o signal peptide | DAKTTQPPSMDCAEGRAANLPCNHSTISGNEYVYWYRQIHSQGPQYIIHGLK | 1075 |
| (IMGT) | NNETNEMASLIITEDRKSSTLILPHATLRDTAVYYCIVRARANAGGTSYGKL | |
| | TFGQGTILTVHP | |
| Vα | MXLVARVTVFLTFGTIIDAKTTQPPSMDCAEGRAANLPCNHSTISGNEYVYW | 1076 |
| | YRQIHSQGPQYIIHGLKNNETNEMASLIITEDRKSSTLILPHATLRDTAVYY | 1077 |
| | CIVRARANAGGTSYGKLTFGQGTILTVHP | |
| | (X = any amino acid) | |
| α chain w/WT signal | MRLVARVTVFLTFGTIIDAKTTQPPSMDCAEGRAANLPCNHSTISGNEYVYW | 1078 |
| peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MALVARVTVFLTFGTIIDAKTTQPPSMDCAEGRAANLPCNHSTISGNEYVYW | 1079 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MHLVARVTVFLTFGTIIDAKTTQPPSMDCAEGRAANLPCNHSTISGNEYVYW | 1080 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1ß | LNHDA | 2071 |
| CDR2ß | SQIVND | 2072 |
| CDR3ß | ASLQFNEQF | 2073 |
| Vß w/o signal peptide | GITQSPKYLFRKEGQNVTLSCEQNLNHDAMYWYRQDPGQGLRLIYYSQIVND | 2074 |
| (SignalP) | FQKGDIAEGYSVSREKKESFPLTVTSAQKNPTAFYLCASLQFNEQFFGPGTR | |
| | LTVL | |
| Vß w/o signal peptide | DGGITQSPKYLFRKEGQNVTLSCEQNLNHDAMYWYRQDPGQGLRLIYYSQIV | 2075 |
| (IMGT) | NDFQKGDIAEGYSVSREKKESFPLTVTSAQKNPTAFYLCASLQFNEQFFGPG | |
| | TRLTVL | |
| Vß | MXMSNQVLCCVVLCFLGANTVDGGITQSPKYLFRKEGQNVTLSCEQNLNHDA | 2076 |
| | MYWYRQDPGQGLRLIYYSQIVNDFQKGDIAEGYSVSREKKESFPLTVTSAQK | |
| | NPTAFYLCASLQFNEQFFGPGTRLTVL | |
| | (X = any amino acid) | |
| Vß (alternative) | MXNQVLCCVVLCFLGANTVDGGITQSPKYLFRKEGQNVTLSCEQNINHDAMY | 2077 |
| | WYRQDPGQGLRLIYYSQIVNDFQKGDIAEGYSVSREKKESFPLTVTSAQKNP | |
| | TAFYLCASLQFNEQFFGPGTRLTVL | |
| | (X = any amino acid) | |
| ß chain w/WT signal | MSMSNQVLCCVVLCFLGANTVDGGITQSPKYLFRKEGQNVTLSCEQNLNHDA | 2078 |
| peptide, Cβ | MYWYRQDPGQGLRLIYYSQIVNDFQKGDIAEGYSVSREKKESFPLTVTSAQK | |
| (substituted) | NPTAFYLCASLQFNEQFFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| ß chain w/alternative | MAMSNQVLCCVVLCFLGANTVDGGITQSPKYLFRKEGQNVTLSCEQNLNHDA | 2079 |
| signal peptide, Cβ | MYWYRQDPGQGLRLIYYSQIVNDFQKGDIAEGYSVSREKKESFPLTVTSAQK | |
| (substituted) | NPTAFYLCASLQFNEQFFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| ß chain w/alternative | MHMSNQVLCCVVLCFLGANTVDGGITQSPKYLFRKEGQNVTLSCEQNLNHDA | 2080 |
| signal peptide, Cβ | MYWYRQDPGQGLRLIYYSQIVNDFQKGDIAEGYSVSREKKESFPLTVTSAQK | |
| (substituted) | NPTAFYLCASLQFNEQFFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR008 interacts with and/or is specific for p53. In some embodiments, the peptide is from a neoantigen of p53. In some embodiments, the neoantigen has the amino acid change R175H relative to the wild type p53 sequence. In some embodiments, TCR008 interacts with the neoantigen in the context of HLA-DRB1*13:01, as described in International Publication No. WO 2020/264269, incorporated herein by reference in its entirety.

TABLE 6I
Amino acid sequences of TCR009.

| | | SEQ |
|---|---|---|
| Description | Sequence | ID NO: |
| CDR1α | SSNFYA | 1081 |
| CDR2α | MTLNGDE | 1082 |
| CDR3α | ALITGGGNKLT | 1083 |
| Va w/o signal peptide | ILNVEQSPQSLHVQEGDSTNFTCSFPSSNFYALHWYRWETAKSPEALFVMTL | 1084 |
| (SignalP) | NGDEKKKGRISATLNTKEGYSYLYIKGSQPEDSATYLCALITGGGNKLTFGT | |
| | GTQLKVEL | |
| Vα w/o signal peptide | ILNVEQSPQSLHVQEGDSTNFTCSFPSSNFYALHWYRWETAKSPEALFVMTL | 1085 |
| (IMGT) | NGDEKKKGRISATLNTKEGYSYLYIKGSQPEDSATYLCALITGGGNKLTFGT | |
| | GTQLKVEL | |
| Vα | MXKNPLAAPLLILWFHLDCVSSILNVEQSPQSLHVQEGDSTNFTCSFPSSNF | 1086 |
| | YALHWYRWETAKSPEALFVMTLNGDEKKKGRISATLNTKEGYSYLYIKGSQP | 1087 |
| | EDSATYLCALITGGGNKLTFGTGTQLKVEL | |
| | (X = any amino acid) | |
| α chain w/WT signal | MEKNPLAAPLLILWFHLDCVSSILNVEQSPQSLHVQEGDSTNFTCSFPSSNF | 1088 |
| peptide, Cα | YALHWYRWETAKSPEALFVMTLNGDEKKKGRISATLNTKEGYSYLYIKGSQP | |
| (substituted) | EDSATYLCALITGGGNKLTFGTGTQLKVELNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain w/alternative | MAKNPLAAPLLILWFHLDCVSSILNVEQSPQSLHVQEGDSTNFTCSFPSSNE | 1089 |
| signal peptide, Cα | YALHWYRWETAKSPEALFVMTLNGDEKKKGRISATLNTKEGYSYLYIKGSQP | |
| (substituted) | EDSATYLCALITGGGNKLTFGTGTQLKVELNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| a chain w/alternative | MHKNPLAAPLLILWFHLDCVSSILNVEQSPQSLHVQEGDSTNFTCSFPSSNF | 1090 |
| signal peptide, Cα | YALHWYRWETAKSPEALFVMTLNGDEKKKGRISATLNTKEGYSYLYIKGSQP | |
| (substituted) | EDSATYLCALITGGGNKLTFGTGTQLKVELNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1ß | MNHEY | 2081 |
| CDR2ß | SVGEGT | 2082 |
| CDR3ß | ASRLQGWNSPLH | 2083 |
| Vß w/o signal peptide | GVTQTPKFRVLKTGQSMTLLCAQDMNHEYMYWYRQDPGMGLRLIHYSVGEGT | 2084 |
| (SignalP) | TAKGEVPDGYNVSRLKKQNFLLGLESAAPSQTSVYFCASRLQGWNSPLHEGN | |
| | GTRLTVT | |
| Vß w/o signal peptide | NAGVTQTPKFRVLKTGQSMTLLCAQDMNHEYMYWYRQDPGMGLRLIHYSVGE | 2085 |
| (IMGT) | GTTAKGEVPDGYNVSRLKKQNFLLGLESAAPSQTSVYFCASRLQGWNSPLHF | |
| | GNGTRLTVT | |
| Vß | MXLGLLCCGAFSLLWAGPVNAGVTQTPKFRVLKTGQSMTLLCAQDMNHEYMY | 2086 |
| | WYRQDPGMGLRLIHYSVGEGTTAKGEVPDGYNVSRLKKQNFLLGLESAAPSQ | 2087 |
| | TSVYFCASRLQGWNSPLHFGNGTRLTVT | |
| | (X = any amino acid) | |
| ß chain w/WT signal | MSLGLLCCGAFSLLWAGPVNAGVTQTPKFRVLKTGQSMTLLCAQDMNHEYMY | 2088 |
| peptide, Cβ | WYRQDPGMGLRLIHYSVGEGTTAKGEVPDGYNVSRLKKQNFLLGLESAAPSQ | |
| (substituted) | TSVYFCASRLQGWNSPLHFGNGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MALGLLCCGAFSLLWAGPVNAGVTQTPKFRVLKTGQSMTLLCAQDMNHEYMY | 2089 |
| signal peptide, Cβ | WYRQDPGMGLRLIHYSVGEGTTAKGEVPDGYNVSRLKKQNFLLGLESAAPSQ | |
| (substituted) | TSVYFCASRLQGWNSPLHFGNGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MHLGLLCCGAFSLLWAGPVNAGVTQTPKFRVLKTGQSMTLLCAQDMNHEYMY | 2090 |
| signal peptide, Cβ | WYRQDPGMGLRLIHYSVGEGTTAKGEVPDGYNVSRLKKQNFLLGLESAAPSQ | |
| (substituted) | TSVYFCASRLQGWNSPLHFGNGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR009 interacts with and/or is specific for p53. In some embodiments, the peptide is from a neoantigen of p53. In some embodiments, the neoantigen has the amino acid change R175H relative to the wild type p53 sequence. In some embodiments, TCR009 interacts with the neoantigen in the context of HLA-DRB1*13:01, as described in

TABLE 6J
Amino acid sequences of TCR010.

| | | SEQ |
|---|---|---|
| | | ID |
| Description | Sequence | NO: |
| CDR1α | TTLSN | 1091 |
| CDR2α | LVKSGEV | 1092 |
| CDR3α | AGPGGAGSYQLT | 1093 |
| Vα w/o signal peptide | QQVMQIPQYQHVQEGEDFTTYCNSSTTLSNIQWYKQRPGGHPVFLIQLVKSG | 1094 |
| (SignalP) | EVKKQKRLTFQFGEAKKNSSLHITATQTTDVGTYFCAGPGGAGSYQLTFGKG | |
| | TKLSVIP | |
| Vα w/o signal peptide | GQQVMQIPQYQHVQEGEDFTTYCNSSTTLSNIQWYKQRPGGHPVFLIQLVKS | 1095 |
| (IMGT) | GEVKKQKRLTFQFGEAKKNSSLHITATQTTDVGTYFCAGPGGAGSYQLTFGK | |
| | GTKLSVIP | |
| Vα | MXLITSMLVLWMQLSQVNGQQVMQIPQYQHVQEGEDFTTYCNSSTTLSNIQW | 1096 |
| | YKQRPGGHPVFLIQLVKSGEVKKQKRLTFQFGEAKKNSSLHITATQTTDVGT | 1097 |
| | YFCAGPGGAGSYQLTFGKGTKLSVIP | |
| | (X = any amino acid) | |
| α chain w/WT signal | MLLITSMLVLWMQLSQVNGQQVMQIPQYQHVQEGEDFTTYCNSSTTLSNIQW | 1098 |
| peptide, Cα | YKQRPGGHPVFLIQLVKSGEVKKQKRLTFQFGEAKKNSSLHITATQTTDVGT | |
| (substituted) | YFCAGPGGAGSYQLTFGKGTKLSVIPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| α chain w/alternative | MALITSMLVLWMQLSQVNGQQVMQIPQYQHVQEGEDFTTYCNSSTTLSNIQW | 1099 |
| signal peptide, Cα | YKQRPGGHPVFLIQLVKSGEVKKQKRLTFQFGEAKKNSSLHITATQTTDVGT | |
| (substituted) | YFCAGPGGAGSYQLTFGKGTKLSVIPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| α chain w/alternative | MHLITSMLVLWMQLSQVNGQQVMQIPQYQHVQEGEDFTTYCNSSTTLSNIQW | 1100 |
| signal peptide, Cα | YKQRPGGHPVFLIQLVKSGEVKKQKRLTFQFGEAKKNSSLHITATQTTDVGT | |
| (substituted) | YFCAGPGGAGSYQLTFGKGTKLSVIPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| CDR1β | MNHEY | 2091 |
| CDR2β | SMNVEV | 2092 |
| CDR3β | ASSPFVVIGQINEQY | 2093 |
| Vβ w/o signal peptide | QVTQNPRYLITVTGKKLTVTCSQNMNHEYMSWYRQDPGLGLRQIYYSMNVEV | 2094 |
| (SignalP) | TDKGDVPEGYKVSRKEKRNFPLILESPSPNQTSLYFCASSPFVVIGQINEQY | |
| | FGPGTRLTVT | |
| Vβ w/o signal peptide | EAQVTQNPRYLITVTGKKLTVTCSQNMNHEYMSWYRQDPGLGLRQIYYSMNV | 2095 |
| (IMGT) | EVTDKGDVPEGYKVSRKEKRNFPLILESPSPNQTSLYFCASSPFVVIGQINE | |
| | QYFGPGTRLTVT | |
| Vβ | MXPQLLGYVVLCLLGAGPLEAQVTQNPRYLITVTGKKLTVTCSQNMNHEYMS | 2096 |
| | WYRQDPGLGLRQIYYSMNVEVTDKGDVPEGYKVSRKEKRNFPLILESPSPNQ | 2097 |
| | TSLYFCASSPFVVIGQINEQYFGPGTRLTVT | |
| | (X = any amino acid) | |
| β chain w/WT signal | MGPQLLGYVVLCLLGAGPLEAQVTQNPRYLITVTGKKLTVTCSQNMNHEYMS | 2098 |
| peptide, Cβ | WYRQDPGLGLRQIYYSMNVEVTDKGDVPEGYKVSRKEKRNFPLILESPSPNQ | |
| (substituted) | TSLYFCASSPFVVIGQINEQYFGPGTRLTVTEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/alternative | MAPQLLGYVVLCLLGAGPLEAQVTQNPRYLITVTGKKLTVTCSQNMNHEYMS | 2099 |
| signal peptide, Cβ | WYRQDPGLGLRQIYYSMNVEVTDKGDVPEGYKVSRKEKRNFPLILESPSPNQ | |
| (substituted) | TSLYFCASSPFVVIGQINEQYFGPGTRLTVTEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/alternative | MHPQLLGYVVLCLLGAGPLEAQVTQNPRYLITVTGKKLTVTCSQNMNHEYMS | 2100 |
| signal peptide, Cβ | WYRQDPGLGLRQIYYSMNVEVTDKGDVPEGYKVSRKEKRNFPLILESPSPNQ | |
| (substituted) | TSLYFCASSPFVVIGQINEQYFGPGTRLTVTEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |

In some embodiments, TCR010 interacts with and/or is specific for p53. In some embodiments, the peptide is from a neoantigen of p53. In some embodiments, the neoantigen has the amino acid change R175H relative to the wild type p53 sequence. In some embodiments, TCR010 interacts with the neoantigen in the context of HLA-DRB1*13:01, as described in International Publication No. WO 2020/264269, incorporated herein by reference in its entirety.

TABLE 6K
Amino acid sequences of TCR011.

| | | SEQ |
|---|---|---|
| | | ID |
| Description | Sequence | NO: |
| CDR1α | TISGNEY | 1101 |
| CDR2α | GLKNN | 1102 |
| CDR3α | IVRARANAGGTSYGKLT | 1103 |
| Vα w/o signal peptide | KTTQPPSMDCAEGRAANLPCNHSTISGNEYVYWYRQIHSQGPQYIIHGLKNN | 1104 |
| (SignalP) | ETNEMASLIITEDRKSSTLILPHATLRDTAVYYCIVRARANAGGTSYGKLTF | |
| | GQGTILTVHP | |
| Vα w/o signal peptide | DAKTTQPPSMDCAEGRAANLPCNHSTISGNEYVYWYRQIHSQGPQYIIHGLK | 1105 |
| (IMGT) | NNETNEMASLIITEDRKSSTLILPHATLRDTAVYYCIVRARANAGGTSYGKL | |
| | TFGQGTILTVHP | |
| Vα | MXLVARVTVFLTFGTIIDAKTTQPPSMDCAEGRAANLPCNHSTISGNEYVYW | 1106 |
| | YRQIHSQGPQYIIHGLKNNETNEMASLIITEDRKSSTLILPHATLRDTAVYY | 1107 |
| | CIVRARANAGGTSYGKLTFGQGTILTVHP | |
| | (X = any amino acid) | |
| α chain w/WT signal | MRLVARVTVFLTFGTIIDAKTTQPPSMDCAEGRAANLPCNHSTISGNEYVYW | 1108 |
| peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain w/alternative | MALVARVTVFLTFGTIIDAKTTQPPSMDCAEGRAANLPCNHSTISGNEYVYW | 1109 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain w/alternative | MHLVARVTVFLTFGTIIDAKTTQPPSMDCAEGRAANLPCNHSTISGNEYVYW | 1110 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| CDR1β | LNHDA | 2101 |
| CDR2β | SQIVND | 2102 |
| CDR3B | ATRTGNEAF | 2103 |
| Vβ w/o signal peptide | GITQSPKYLFRKEGQNVTLSCEQNLNHDAMYWYRQDPGQGLRLIYYSQIVND | 2104 |
| (SignalP) | FQKGDIAEGYSVSREKKESFPLTVTSAQKNPTAFYLCATRTGNEAFFGQGTR | |
| | LTVV | |
| Vβ w/o signal peptide | DGGITQSPKYLFRKEGQNVTLSCEQNLNHDAMYWYRQDPGQGLRLIYYSQIV | 2105 |
| (IMGT) | NDFQKGDIAEGYSVSREKKESFPLTVTSAQKNPTAFYLCATRTGNEAFFGQG | |
| | TRLTVV | |
| Vβ | MXNQVLCCVVLCFLGANTVDGGITQSPKYLFRKEGQNVTLSCEQNLNHDAMY | 2106 |
| | WYRQDPGQGLRLIYYSQIVNDFQKGDIAEGYSVSREKKESFPLTVTSAQKNP | 2107 |
| | TAFYLCATRTGNEAFFGQGTRLTVV | |
| | (X = any amino acid) | |
| β chain w/WT signal | MSNQVLCCVVLCFLGANTVDGGITQSPKYLFRKEGQNVTLSCEQNLNHDAMY | 2108 |
| peptide, Cβ | WYRQDPGQGLRLIYYSQIVNDFQKGDIAEGYSVSREKKESFPLTVTSAQKNP | |
| (substituted) | TAFYLCATRTGNEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain w/alternative | MANQVLCCVVLCFLGANTVDGGITQSPKYLFRKEGQNVTLSCEQNLNHDAMY | 2109 |
| signal peptide, Cβ | WYRQDPGQGLRLIYYSQIVNDFQKGDIAEGYSVSREKKESFPLTVTSAQKNP | |
| (substituted) | TAFYLCATRTGNEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain w/alternative | MHNQVLCCVVLCFLGANTVDGGITQSPKYLFRKEGQNVTLSCEQNLNHDAMY | 2110 |
| signal peptide, Cβ | WYRQDPGQGLRLIYYSQIVNDFQKGDIAEGYSVSREKKESFPLTVTSAQKNP | |
| (substituted) | TAFYLCATRTGNEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR011 interacts with and/or is specific for p53. In some embodiments, the peptide is from a neoantigen of p53. In some embodiments, the neoantigen has the amino acid change R175H relative to the wild type p53 sequence. In some embodiments, TCR011 interacts with the neoantigen in the context of HLA-DRB1*13:01, as described in International Publication No. WO 2020/264269, incorporated herein by reference in its entirety.

TABLE 6L
Amino acid sequences of TCR012.

| | | SEQ |
|---|---|---|
| | | ID |
| Description | Sequence | NO: |
| CDR1α | VSNAYN | 1111 |
| CDR2α | GSKP | 1112 |
| CDR3α | AVEDRRRTALI | 1113 |
| Vα w/o signal peptide | KDQVFQPSTVASSEGAVVEIFCNHSVSNAYNFFWYLHFPGCAPRLLVKGSKP | 1114 |
| (SignalP) | SQQGRYNMTYERFSSSLLILQVREADAAVYYCAVEDRRRTALIFGKGTTLSV | |
| | SS | |
| Vα w/o signal peptide | KDQVFQPSTVASSEGAVVEIFCNHSVSNAYNFFWYLHFPGCAPRLLVKGSKP | 1115 |
| (IMGT) | SQQGRYNMTYERFSSSLLILQVREADAAVYYCAVEDRRRTALIFGKGTTLSV | |
| | SS | |
| Vα | MXLQSTLGAVWLGLLLNSLWKVAESKDQVFQPSTVASSEGAVVEIFCNHSVS | 1116 |
| | NAYNFFWYLHFPGCAPRLLVKGSKPSQQGRYNMTYERFSSSLLILQVREADA | 1117 |
| | AVYYCAVEDRRRTALIFGKGTTLSVSS | |
| | (X = any amino acid) | |
| α chain w/WT signal | MALQSTLGAVWLGLLLNSLWKVAESKDQVFQPSTVASSEGAVVEIFCNHSVS | 1118 |
| peptide, Cα | NAYNFFWYLHFPGCAPRLLVKGSKPSQQGRYNMTYERFSSSLLILQVREADA | |
| | AVYYCAVEDRRRTALIFGKGTTLSVSSNIQNPEPAVYQLKDPRSQDSTLCLF | |
| (substituted) | TDFDSQINVPKTMESGTFITDKCVLDMKAMDSKSNGAIAWSNQTSFTCQDIF | 1119 |
| | KETNATYPSSDVPCDATLTEKSFETDMNLNFQNLLVIVLRILLLKVAGENLL | |
| | MTLRLWSS | |
| α chain w/alternative | MHLQSTLGAVWLGLLLNSLWKVAESKDQVFQPSTVASSEGAVVEIFCNHSVS | 1120 |
| signal peptide, Cα | NAYNFFWYLHFPGCAPRLLVKGSKPSQQGRYNMTYERFSSSLLILQVREADA | |
| (substituted) | AVYYCAVEDRRRTALIFGKGTTLSVSSNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| CDR1β | SNHLY | 2111 |
| CDR2β | FYNNEI | 2112 |
| CDR3B | ASSEYQSQSNEQF | 2113 |
| Vβ w/o signal peptide | EPEVTQTPSHQVTQMGQEVILRCVPISNHLYFYWYRQILGQKVEFLVSFYNN | 2114 |
| (SignalP) | EISEKSEIFDDQFSVERPDGSNFTLKIRSTKLEDSAMYFCASSEYQSQSNEQ | |
| | FFGPGTRLTVL | |
| Vβ w/o signal peptide | EPEVTQTPSHQVTQMGQEVILRCVPISNHLYFYWYRQILGQKVEFLVSFYNN | 2115 |
| (IMGT) | EISEKSEIFDDQFSVERPDGSNFTLKIRSTKLEDSAMYFCASSEYQSQSNEQ | |
| | FFGPGTRLTVL | |
| Vβ | MXTWLVCWAIFSLLKAGLTEPEVTQTPSHQVTQMGQEVILRCVPISNHLYFY | 2116 |
| | WYRQILGQKVEFLVSFYNNEISEKSEIFDDQFSVERPDGSNFTLKIRSTKLE | 2117 |
| | DSAMYFCASSEYQSQSNEQFFGPGTRLTVL | |
| | (X = any amino acid) | |
| β chain w/WT signal | MDTWLVCWAIFSLLKAGLTEPEVTQTPSHQVTQMGQEVILRCVPISNHLYFY | 2118 |
| peptide, Cβ | WYRQILGQKVEFLVSFYNNEISEKSEIFDDQFSVERPDGSNFTLKIRSTKLE | |
| (substituted) | DSAMYFCASSEYQSQSNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MATWLVCWAIFSLLKAGLTEPEVTQTPSHQVTQMGQEVILRCVPISNHLYFY | 2119 |
| signal peptide, Cβ | WYRQILGQKVEFLVSFYNNEISEKSEIFDDQFSVERPDGSNFTLKIRSTKLE | |
| (substituted) | DSAMYFCASSEYQSQSNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MHTWLVCWAIFSLLKAGLTEPEVTQTPSHQVTQMGQEVILRCVPISNHLYFY | 2120 |
| signal peptide, Cβ | WYRQILGQKVEFLVSFYNNEISEKSEIFDDQFSVERPDGSNFTLKIRSTKLE | |
| (substituted) | DSAMYFCASSEYQSQSNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR012 interacts with and/or is specific for p53. In some embodiments, the peptide is from a neoantigen of p53. In some embodiments, the neoantigen has the amino acid change R175H relative to the wild type p53 sequence. In some embodiments, TCR012 interacts with the neoantigen in the context of HLA-DRB1*13:01, as described in International Publication No. WO 2020/264269, incorporated herein by reference in its entirety.

TABLE 6M
Amino acid sequences of TCR013.

| | | SEQ |
|---|---|---|
| | | ID |
| Description | Sequence | NO: |
| CDR1α | TISGTDY | 1121 |
| CDR2α | GLTSN | 1122 |
| CDR3α | ILRDNNARLM | 1123 |
| Vα w/o signal peptide | KTTQPNSMESNEEEPVHLPCNHSTISGTDYIHWYRQLPSQGPEYVIHGLTSN | 1124 |
| (SignalP) | VNNRMASLAIAEDRKSSTLILHRATLRDAAVYYCILRDNNARLMFGDGTQLV | |
| | VKP | |
| Vα w/o signal peptide | DAKTTQPNSMESNEEEPVHLPCNHSTISGTDYIHWYRQLPSQGPEYVIHGLT | 1125 |
| (IMGT) | SNVNNRMASLAIAEDRKSSTLILHRATLRDAAVYYCILRDNNARLMFGDGTQ | |
| | LVVKP | |
| Vα | MXLVTSITVLLSLGIMGDAKTTQPNSMESNEEEPVHLPCNHSTISGTDYIHW | 1126 |
| | YRQLPSQGPEYVIHGLTSNVNNRMASLAIAEDRKSSTLILHRATLRDAAVYY | 1127 |
| | CILRDNNARLMFGDGTQLVVKP | |
| | (X = any amino acid) | |
| α chain w/WT signal | MKLVTSITVLLSLGIMGDAKTTQPNSMESNEEEPVHLPCNHSTISGTDYIHW | 1128 |
| peptide, Cα | YRQLPSQGPEYVIHGLTSNVNNRMASLAIAEDRKSSTLILHRATLRDAAVYY | |
| (substituted) | CILRDNNARLMFGDGTQLVVKPNIQNPEPAVYQLKDPRSQDSTLCLFTDFDS | |
| | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGENLLMTLRL | |
| | WSS | |
| α chain w/alternative | MALVTSITVLLSLGIMGDAKTTQPNSMESNEEEPVHLPCNHSTISGTDYIHW | 1129 |
| signal peptide, Cα | YRQLPSQGPEYVIHGLTSNVNNRMASLAIAEDRKSSTLILHRATLRDAAVYY | |
| (substituted) | CILRDNNARLMFGDGTQLVVKPNIQNPEPAVYQLKDPRSQDSTLCLFTDEDS | |
| | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGENLLMTLRL | |
| | WSS | |
| α chain w/alternative | MHLVTSITVLLSLGIMGDAKTTQPNSMESNEEEPVHLPCNHSTISGTDYIHW | 1130 |
| signal peptide, Cα | YRQLPSQGPEYVIHGLTSNVNNRMASLAIAEDRKSSTLILHRATLRDAAVYY | |
| (substituted) | CILRDNNARLMFGDGTQLVVKPNIQNPEPAVYQLKDPRSQDSTLCLFTDEDS | |
| | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGENLLMTLRL | |
| | WSS | |
| CDR1β | MNHEY | 2121 |
| CDR2β | SMNVEV | 2122 |
| CDR3β | ASGLVGFNQPQH | 2123 |
| Vβ w/o signal peptide | QVTQNPRYLITVTGKKLTVTCSQNMNHEYMSWYRQDPGLGLRQIYYSMNVEV | 2124 |
| (SignalP) | TDKGDVPEGYKVSRKEKRNFPLILESPSPNQTSLYFCASGLVGFNQPQHFGD | |
| | GTRLSIL | |
| Vβ w/o signal peptide | EAQVTQNPRYLITVTGKKLTVTCSQNMNHEYMSWYRQDPGLGLRQIYYSMNV | 2125 |
| (IMGT) | EVTDKGDVPEGYKVSRKEKRNFPLILESPSPNQTSLYFCASGLVGFNQPQHE | |
| | GDGTRLSIL | |
| Vβ | MXPQLLGYVVLCLLGAGPLEAQVTQNPRYLITVTGKKLTVTCSQNMNHEYMS | 2126 |
| | WYRQDPGLGLRQIYYSMNVEVTDKGDVPEGYKVSRKEKRNFPLILESPSPNQ | 2127 |
| | TSLYFCASGLVGFNQPQHFGDGTRLSIL | |
| | (X = any amino acid) | |
| β chain w/WT signal | MGPQLLGYVVLCLLGAGPLEAQVTQNPRYLITVTGKKLTVTCSQNMNHEYMS | 2128 |
| peptide, Cβ | WYRQDPGLGLRQIYYSMNVEVTDKGDVPEGYKVSRKEKRNFPLILESPSPNQ | |
| (substituted) | TSLYFCASGLVGFNQPQHFGDGTRLSILEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAPQLLGYVVLCLLGAGPLEAQVTQNPRYLITVTGKKLTVTCSQNMNHEYMS | 2129 |
| signal peptide, Cβ | WYRQDPGLGLRQIYYSMNVEVTDKGDVPEGYKVSRKEKRNFPLILESPSPNQ | |
| (substituted) | TSLYFCASGLVGFNQPQHFGDGTRLSILEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHPQLLGYVVLCLLGAGPLEAQVTQNPRYLITVTGKKLTVTCSQNMNHEYMS | 2130 |
| signal peptide, Cβ | WYRQDPGLGLRQIYYSMNVEVTDKGDVPEGYKVSRKEKRNFPLILESPSPNQ | |
| (substituted) | TSLYFCASGLVGFNQPQHFGDGTRLSILEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR013 interacts with and/or is specific for p53. In some embodiments, the peptide is from a neoantigen of p53. In some embodiments, the neoantigen has the amino acid change R175H relative to the wild type p53 sequence. In some embodiments, TCR013 interacts with the neoantigen in the context of HLA-DRB1*13:01, as described in International Publication No. WO 2020/264269, incorporated herein by reference in its entirety.

TABLE 6N
Amino acid sequences of TCR014

| | | SEQ |
|---|---|---|
| | | ID |
| Description | Sequence | NO: |
| CDR1α | VSGNPY | 1131 |
| CDR2α | YITGDNLV | 1132 |
| CDR3α | AVRDGSATSGTYKYI | 1133 |
| Vα w/o signal peptide | QSVAQPEDQVNVAEGNPLTVKCTYSVSGNPYLFWYVQYPNRGLQFLLKYITG | 1134 |
| (SignalP) | DNLVKGSYGFEAEFNKSQTSFHLKKPSALVSDSALYFCAVRDGSATSGTYKY | |
| | IFGTGTRLKVLA | |
| Vα w/o signal peptide | AQSVAQPEDQVNVAEGNPLTVKCTYSVSGNPYLFWYVQYPNRGLQFLLKYIT | 1135 |
| (IMGT) | GDNLVKGSYGFEAEFNKSQTSFHLKKPSALVSDSALYFCAVRDGSATSGTYK | |
| | YIFGTGTRLKVLA | |
| Vα | MXSAPISMLAMLFTLSGLRAQSVAQPEDQVNVAEGNPLTVKCTYSVSGNPYL | 1136 |
| | FWYVQYPNRGLQFLLKYITGDNLVKGSYGFEAEFNKSQTSFHLKKPSALVSD | 1137 |
| | SALYFCAVRDGSATSGTYKYIFGTGTRLKVLA | |
| | (X = any amino acid) | |
| α chain w/WT signal | MASAPISMLAMLFTLSGLRAQSVAQPEDQVNVAEGNPLTVKCTYSVSGNPYL | 1138 |
| peptide, Cα | FWYVQYPNRGLQFLLKYITGDNLVKGSYGFEAEFNKSQTSFHLKKPSALVSD | |
| | SALYFCAVRDGSATSGTYKYIFGTGTRLKVLANIQNPEPAVYQLKDPRSQDS | |
| (substituted) | TLCLFTDFDSQINVPKTMESGTFITDKCVLDMKAMDSKSNGAIAWSNQTSFT | 1139 |
| | CQDIFKETNATYPSSDVPCDATLTEKSFETDMNLNFQNLLVIVLRILLLKVA | |
| | GFNLLMTLRLWSS | |
| α chain w/alternative | MHSAPISMLAMLFTLSGLRAQSVAQPEDQVNVAEGNPLTVKCTYSVSGNPYL | 1140 |
| signal peptide, Cα | FWYVQYPNRGLQFLLKYITGDNLVKGSYGFEAEFNKSQTSFHLKKPSALVSD | |
| (substituted) | SALYFCAVRDGSATSGTYKYIFGTGTRLKVLANIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1β | SEHNR | 2131 |
| CDR2β | FQNEAQ | 2132 |
| CDR3β | ASSPGLAYEQY | 2133 |
| Vβ w/o signal peptide | DTGVSQDPRHKITKRGQNVTFRCDPISEHNRLYWYRQTLGQGPEFLTYFQNE | 2134 |
| (SignalP) | AQLEKSRLLSDRFSAERPKGSFSTLEIQRTEQGDSAMYLCASSPGLAYEQYF | |
| | GPGTRLTVT | |
| Vβ w/o signal peptide | DTGVSQDPRHKITKRGQNVTFRCDPISEHNRLYWYRQTLGQGPEFLTYFQNE | 2135 |
| (IMGT) | AQLEKSRLLSDRESAERPKGSESTLEIQRTEQGDSAMYLCASSPGLAYEQYF | |
| | GPGTRLTVT | |
| Vβ | MXTSLLCWMALCLLGADHADTGVSQDPRHKITKRGQNVTFRCDPISEHNRLY | 2136 |
| | WYRQTLGQGPEFLTYFQNEAQLEKSRLLSDRFSAERPKGSFSTLEIQRTEQG | 2137 |
| | DSAMYLCASSPGLAYEQYFGPGTRLTVT | |
| | (X = any amino acid) | |
| β chain w/WT signal | MGTSLLCWMALCLLGADHADTGVSQDPRHKITKRGQNVTFRCDPISEHNRLY | 2138 |
| peptide, Cβ | WYRQTLGQGPEFLTYFQNEAQLEKSRLLSDRESAERPKGSESTLEIQRTEQG | |
| (substituted) | DSAMYLCASSPGLAYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MATSLLCWMALCLLGADHADTGVSQDPRHKITKRGQNVTFRCDPISEHNRLY | 2139 |
| signal peptide, Cβ | WYRQTLGQGPEFLTYFQNEAQLEKSRLLSDRESAERPKGSESTLEIQRTEQG | |
| (substituted) | DSAMYLCASSPGLAYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHTSLLCWMALCLLGADHADTGVSQDPRHKITKRGQNVTFRCDPISEHNRLY | 2140 |
| signal peptide, Cβ | WYRQTLGQGPEFLTYFQNEAQLEKSRLLSDRESAERPKGSESTLEIQRTEQG | |
| (substituted) | DSAMYLCASSPGLAYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR014 interacts with and/or is specific for p53. In some embodiments, the peptide is from a neoantigen of p53. In some embodiments, the neoantigen has the amino acid change Y220C relative to the wild type p53 sequence. In some embodiments, TCR014 interacts with the neoantigen in the context of HLA-A*02:01, as described in International Publication No. WO 2020/264269, incorporated herein by reference in its entirety.

TABLE 6O
Amino acid sequences of TCR015.

| | | SEQ |
|---|---|---|
| | | ID |
| Description | Sequence | NO: |
| CDR1α | DRGSQS | 1141 |
| CDR2α | IYSNGD | 1142 |
| CDR3α | AWNSGGSNYKLT | 1143 |
| Vα w/o signal peptide | QQKEVEQNSGPLSVPEGAIASLNCTYSDRGSQSFFWYRQYSGKSPELIMFIY | 1144 |
| (SignalP) | SNGDKEDGRFTAQLNKASQYVSLLIRDSQPSDSATYLCAWNSGGSNYKLTFG | |
| | KGTLLTVNP | |
| Vα w/o signal peptide | QKEVEQNSGPLSVPEGAIASLNCTYSDRGSQSFFWYRQYSGKSPELIMFIYS | 1145 |
| (IMGT) | NGDKEDGRFTAQLNKASQYVSLLIRDSQPSDSATYLCAWNSGGSNYKLTFGK | |
| | GTLLTVNP | |
| Vα | MXSLRVLLVILWLQLSWVWSQQKEVEQNSGPLSVPEGAIASLNCTYSDRGSQ | 1146 |
| | SFFWYRQYSGKSPELIMFIYSNGDKEDGRFTAQLNKASQYVSLLIRDSQPSD | 1147 |
| | SATYLCAWNSGGSNYKLTFGKGTLLTVNP | |
| | (X = any amino acid) | |
| α chain w/WT signal | MKSLRVLLVILWLQLSWVWSQQKEVEQNSGPLSVPEGAIASLNCTYSDRGSQ | 1148 |
| peptide, Cα | SFFWYRQYSGKSPELIMFIYSNGDKEDGRFTAQLNKASQYVSLLIRDSQPSD | |
| (substituted) | SATYLCAWNSGGSNYKLTFGKGTLLTVNPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain w/alternative | MASLRVLLVILWLQLSWVWSQQKEVEQNSGPLSVPEGAIASLNCTYSDRGSQ | 1149 |
| signal peptide, Cα | SFFWYRQYSGKSPELIMFIYSNGDKEDGRFTAQLNKASQYVSLLIRDSQPSD | |
| (substituted) | SATYLCAWNSGGSNYKLTFGKGTLLTVNPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain w/alternative | MHSLRVLLVILWLQLSWVWSQQKEVEQNSGPLSVPEGAIASLNCTYSDRGSQ | 1150 |
| signal peptide, Cα | SFFWYRQYSGKSPELIMFIYSNGDKEDGRFTAQLNKASQYVSLLIRDSQPSD | |
| (substituted) | SATYLCAWNSGGSNYKLTFGKGTLLTVNPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| CDR1β | MNHEY | 2141 |
| CDR2β | SVGEGT | 2142 |
| CDR3β | ASSYSQAWGQPQH | 2143 |
| Vβ w/o signal peptide | GVTQTPKFRVLKTGQSMTLLCAQDMNHEYMYWYRQDPGMGLRLIHYSVGEGT | 2144 |
| (SignalP) | TAKGEVPDGYNVSRLKKQNFLLGLESAAPSQTSVYFCASSYSQAWGQPQHFG | |
| | DGTRLSIL | |
| Vβ w/o signal peptide | NAGVTQTPKFRVLKTGQSMTLLCAQDMNHEYMYWYRQDPGMGLRLIHYSVGE | 2145 |
| (IMGT) | GTTAKGEVPDGYNVSRLKKQNFLLGLESAAPSQTSVYFCASSYSQAWGQPQH | |
| | FGDGTRLSIL | |
| Vβ | MXLGLLCCGAFSLLWAGPVNAGVTQTPKFRVLKTGQSMTLLCAQDMNHEYMY | 2146 |
| | WYRQDPGMGLRLIHYSVGEGTTAKGEVPDGYNVSRLKKQNFLLGLESAAPSQ | 2147 |
| | TSVYFCASSYSQAWGQPQHFGDGTRLSIL | |
| | (X = any amino acid) | |
| β chain w/WT signal | MSLGLLCCGAFSLLWAGPVNAGVTQTPKFRVLKTGQSMTLLCAQDMNHEYMY | 2148 |
| peptide, Cβ | WYRQDPGMGLRLIHYSVGEGTTAKGEVPDGYNVSRLKKQNFLLGLESAAPSQ | |
| (substituted) | TSVYFCASSYSQAWGQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MALGLLCCGAFSLLWAGPVNAGVTQTPKFRVLKTGQSMTLLCAQDMNHEYMY | 2149 |
| signal peptide, Cβ | WYRQDPGMGLRLIHYSVGEGTTAKGEVPDGYNVSRLKKQNFLLGLESAAPSQ | |
| (substituted) | TSVYFCASSYSQAWGQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MHLGLLCCGAFSLLWAGPVNAGVTQTPKFRVLKTGQSMTLLCAQDMNHEYMY | 2150 |
| signal peptide, Cβ | WYRQDPGMGLRLIHYSVGEGTTAKGEVPDGYNVSRLKKQNFLLGLESAAPSQ | |
| (substituted) | TSVYFCASSYSQAWGQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR015 interacts with and/or is specific for p53. In some embodiments, the peptide is from a neoantigen of p53. In some embodiments, the neoantigen has the amino acid change Y220C relative to the wild type p53 sequence. In some embodiments, TCR015 interacts with the neoantigen in the context of HLA-DRB1*04:01:01, as described in

TABLE 6P
Amino acid sequences of TCR016.

| | | SEQ |
|---|---|---|
| | | ID |
| Description | Sequence | NO: |
| CDR1α | VSGNPY | 1151 |
| CDR2α | YITGDNLV | 1152 |
| CDR3α | AVRVWDYKLS | 1153 |
| Vα w/o signal peptide | QSVAQPEDQVNVAEGNPLTVKCTYSVSGNPYLFWYVQYPNRGLQFLLKYITG | 1154 |
| (SignalP) | DNLVKGSYGFEAEFNKSQTSFHLKKPSALVSDSALYFCAVRVWDYKLSFGAG | |
| | TTVTVRA | |
| Vα w/o signal peptide | AQSVAQPEDQVNVAEGNPLTVKCTYSVSGNPYLFWYVQYPNRGLQFLLKYIT | 1155 |
| (IMGT) | GDNLVKGSYGFEAEFNKSQTSFHLKKPSALVSDSALYFCAVRVWDYKLSFGA | |
| | GTTVTVRA | |
| Vα | MXSAPISMLAMLFTLSGLRAQSVAQPEDQVNVAEGNPLTVKCTYSVSGNPYL | 1156 |
| | FWYVQYPNRGLQFLLKYITGDNLVKGSYGFEAEFNKSQTSFHLKKPSALVSD | 1157 |
| | SALYFCAVRVWDYKLSFGAGTTVTVRA | |
| | (X = any amino acid) | |
| α chain w/WT signal | MASAPISMLAMLFTLSGLRAQSVAQPEDQVNVAEGNPLTVKCTYSVSGNPYL | 1158 |
| peptide, Cα | FWYVQYPNRGLQFLLKYITGDNLVKGSYGFEAEFNKSQTSFHLKKPSALVSD | |
| | SALYFCAVRVWDYKLSFGAGTTVTVRANIQNPEPAVYQLKDPRSQDSTLCLF | |
| (substituted) | TDFDSQINVPKTMESGTFITDKCVLDMKAMDSKSNGAIAWSNQTSFTCQDIF | 1159 |
| | KETNATYPSSDVPCDATLTEKSFETDMNLNFQNLLVIVLRILLLKVAGENLL | |
| | MTLRLWSS | |
| α chain w/alternative | MHSAPISMLAMLFTLSGLRAQSVAQPEDQVNVAEGNPLTVKCTYSVSGNPYL | 1160 |
| signal peptide, Cα | FWYVQYPNRGLQFLLKYITGDNLVKGSYGFEAEFNKSQTSFHLKKPSALVSD | |
| (substituted) | SALYFCAVRVWDYKLSFGAGTTVTVRANIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| CDR1β | LNHDA | 2151 |
| CDR2β | SQIVND | 2152 |
| CDR3β | ASSISAGGDGYT | 2153 |
| Vβ w/o signal peptide | GITQSPKYLFRKEGQNVTLSCEQNLNHDAMYWYRQDPGQGLRLIYYSQIVND | 2154 |
| (SignalP) | FQKGDIAEGYSVSREKKESFPLTVTSAQKNPTAFYLCASSISAGGDGYTFGS | |
| | GTRLTVV | |
| Vβ w/o signal peptide | DGGITQSPKYLFRKEGQNVTLSCEQNLNHDAMYWYRQDPGQGLRLIYYSQIV | 2155 |
| (IMGT) | NDFQKGDIAEGYSVSREKKESFPLTVTSAQKNPTAFYLCASSISAGGDGYTF | |
| | GSGTRLTVV | |
| Vβ | MXNQVLCCVVLCFLGANTVDGGITQSPKYLFRKEGQNVTLSCEQNLNHDAMY | 2156 |
| | WYRQDPGQGLRLIYYSQIVNDFQKGDIAEGYSVSREKKESFPLTVTSAQKNP | 2157 |
| | TAFYLCASSISAGGDGYTFGSGTRLTVV | |
| | (X = any amino acid) | |
| β chain w/WT signal | MSNQVLCCVVLCFLGANTVDGGITQSPKYLFRKEGQNVTLSCEQNLNHDAMY | 2158 |
| peptide, Cβ | WYRQDPGQGLRLIYYSQIVNDFQKGDIAEGYSVSREKKESFPLTVTSAQKNP | |
| (substituted) | TAFYLCASSISAGGDGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MANQVLCCVVLCFLGANTVDGGITQSPKYLFRKEGQNVTLSCEQNLNHDAMY | 2159 |
| signal peptide, Cβ | WYRQDPGQGLRLIYYSQIVNDFQKGDIAEGYSVSREKKESFPLTVTSAQKNP | |
| (substituted) | TAFYLCASSISAGGDGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHNQVLCCVVLCFLGANTVDGGITQSPKYLFRKEGQNVTLSCEQNLNHDAMY | 2160 |
| signal peptide, Cβ | WYRQDPGQGLRLIYYSQIVNDFQKGDIAEGYSVSREKKESFPLTVTSAQKNP | |
| (substituted) | TAFYLCASSISAGGDGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR016 interacts with and/or is specific for p53. In some embodiments, the peptide is from a neoantigen of p53. In some embodiments, the neoantigen has the amino acid change Y220C relative to the wild type p53 sequence. In some embodiments, TCR016 interacts with the neoantigen in the context of HLA-DRB3*02:02, as described in International Publication No. WO 2019/067243, incorporated herein by reference in its entirety.

TABLE 6Q
Amino acid sequences of TCR017.

| | | SEQ |
|---|---|---|
| | | ID |
| Description | Sequence | NO: |
| CDR1α | TSGENG | 1161 |
| CDR2α | NVLDGL | 1162 |
| CDR3α | AVKWTGGFKTI | 1163 |
| Vα w/o signal peptide | QNIDQPTEMTATEGAIVQINCTYQTSGENGLFWYQQHAGEAPTFLSYNVLDG | 1164 |
| (SignalP) | LEEKGRESSFLSRSKGYSYLLLKELQMKDSASYLCAVKWTGGFKTIFGAGTR | |
| | LFVKA | |
| Vα w/o signal peptide | GQNIDQPTEMTATEGAIVQINCTYQTSGENGLFWYQQHAGEAPTFLSYNVLD | 1165 |
| (IMGT) | GLEEKGRESSFLSRSKGYSYLLLKELQMKDSASYLCAVKWTGGFKTIFGAGT | |
| | RLFVKA | |
| Vα | MXGVFLLYVSMKMGGTTGQNIDQPTEMTATEGAIVQINCTYQTSGENGLFWY | 1166 |
| | QQHAGEAPTFLSYNVLDGLEEKGRESSFLSRSKGYSYLLLKELQMKDSASYL | 1167 |
| | CAVKWTGGFKTIFGAGTRLFVKA | |
| | (X = any amino acid) | |
| α chain w/WT signal | MWGVFLLYVSMKMGGTTGQNIDQPTEMTATEGAIVQINCTYQTSGENGLFWY | 1168 |
| peptide, Cα | QQHAGEAPTFLSYNVLDGLEEKGRESSFLSRSKGYSYLLLKELQMKDSASYL | |
| (substituted) | CAVKWTGGFKTIFGAGTRLFVKANIQNPEPAVYQLKDPRSQDSTLCLFTDED | |
| | SQINVPKTMESGTFITDKCVLDMKAMDSKSNGAIAWSNQTSFTCQDIFKETN | |
| | ATYPSSDVPCDATLTEKSFETDMNLNFQNLLVIVLRILLLKVAGENLLMTLR | |
| | LWSS | |
| α chain w/alternative | MAGVFLLYVSMKMGGTTGQNIDQPTEMTATEGAIVQINCTYQTSGENGLFWY | 1169 |
| signal peptide, Cα | QQHAGEAPTFLSYNVLDGLEEKGRESSFLSRSKGYSYLLLKELQMKDSASYL | |
| (substituted) | CAVKWTGGFKTIFGAGTRLFVKANIQNPEPAVYQLKDPRSQDSTLCLFTDED | |
| | SQINVPKTMESGTFITDKCVLDMKAMDSKSNGAIAWSNQTSFTCQDIFKETN | |
| | ATYPSSDVPCDATLTEKSFETDMNLNFQNLLVIVLRILLLKVAGENLLMTLR | |
| | LWSS | |
| α chain w/alternative | MHGVFLLYVSMKMGGTTGQNIDQPTEMTATEGAIVQINCTYQTSGENGLFWY | 1170 |
| signal peptide, Cα | QQHAGEAPTFLSYNVLDGLEEKGRESSFLSRSKGYSYLLLKELQMKDSASYL | |
| (substituted) | CAVKWTGGFKTIFGAGTRLFVKANIQNPEPAVYQLKDPRSQDSTLCLFTDED | |
| | SQINVPKTMESGTFITDKCVLDMKAMDSKSNGAIAWSNQTSFTCQDIFKETN | |
| | ATYPSSDVPCDATLTEKSFETDMNLNFQNLLVIVLRILLLKVAGENLLMTLR | |
| | LWSS | |
| CDR1β | MNHEY | 2161 |
| CDR2β | SVGAGI | 2162 |
| CDR3B | ASSYRESHYGYT | 2163 |
| Vβ w/o signal peptide | GVTQTPKFQVLKTGQSMTLQCAQDMNHEYMSWYRQDPGMGLRLIHYSVGAGI | 2164 |
| (SignalP) | TDQGEVPNGYNVSRSTTEDFPLRLLSAAPSQTSVYFCASSYRESHYGYTFGS | |
| | GTRLTVV | |
| Vβ w/o signal peptide | NAGVTQTPKFQVLKTGQSMTLQCAQDMNHEYMSWYRQDPGMGLRLIHYSVGA | 2165 |
| (IMGT) | GITDQGEVPNGYNVSRSTTEDFPLRLLSAAPSQTSVYFCASSYRESHYGYTF | |
| | GSGTRLTVV | |
| Vβ | MXIGLLCCAALSLLWAGPVNAGVTQTPKFQVLKTGQSMTLQCAQDMNHEYMS | 2166 |
| | WYRQDPGMGLRLIHYSVGAGITDQGEVPNGYNVSRSTTEDFPLRLLSAAPSQ | 2167 |
| | TSVYFCASSYRESHYGYTFGSGTRLTVV | |
| | (X = any amino acid) | |
| β chain w/WT signal | MSIGLLCCAALSLLWAGPVNAGVTQTPKFQVLKTGQSMTLQCAQDMNHEYMS | 2168 |
| peptide, Cβ | WYRQDPGMGLRLIHYSVGAGITDQGEVPNGYNVSRSTTEDEPLRLLSAAPSQ | |
| (substituted) | TSVYFCASSYRESHYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAIGLLCCAALSLLWAGPVNAGVTQTPKFQVLKTGQSMTLQCAQDMNHEYMS | 2169 |
| signal peptide, Cβ | WYRQDPGMGLRLIHYSVGAGITDQGEVPNGYNVSRSTTEDFPLRLLSAAPSQ | |
| (substituted) | TSVYFCASSYRESHYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHIGLLCCAALSLLWAGPVNAGVTQTPKFQVLKTGQSMTLQCAQDMNHEYMS | 2170 |
| signal peptide, Cβ | WYRQDPGMGLRLIHYSVGAGITDQGEVPNGYNVSRSTTEDFPLRLLSAAPSQ | |
| (substituted) | TSVYFCASSYRESHYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR017 interacts with and/or is specific for p53. In some embodiments, the peptide is from a neoantigen of p53. In some embodiments, the neoantigen has the amino acid change G245S relative to the wild type p53 sequence. In some embodiments, TCR017 interacts with the neoantigen in the context of HLA-DRB3*02:02, as described in

TABLE 6R
Amino acid sequences of TCR018.

| | | SEQ |
|---|---|---|
| | | ID |
| Description | Sequence | NO: |
| CDR1α | YGGTVN | 1171 |
| CDR2α | YFSGDPLV | 1172 |
| CDR3α | AVKGDYKLS | 1173 |
| Vα w/o signal peptide | QSVSQHNHHVILSEAASLELGCNYSYGGTVNLFWYVQYPGQHLQLLLKYFSG | 1174 |
| (SignalP) | DPLVKGIKGFEAEFIKSKFSFNLRKPSVQWSDTAEYFCAVKGDYKLSFGAGT | |
| | TVTVRA | |
| Vα w/o signal peptide | AQSVSQHNHHVILSEAASLELGCNYSYGGTVNLFWYVQYPGQHLQLLLKYFS | 1175 |
| (IMGT) | GDPLVKGIKGFEAEFIKSKFSFNLRKPSVQWSDTAEYFCAVKGDYKLSFGAG | |
| | TTVTVRA | |
| Vα | MXLLLIPVLGMIFALRDARAQSVSQHNHHVILSEAASLELGCNYSYGGTVNL | 1176 |
| | FWYVQYPGQHLQLLLKYFSGDPLVKGIKGFEAEFIKSKFSFNLRKPSVQWSD | 1177 |
| | TAEYFCAVKGDYKLSFGAGTTVTVRA | |
| | (X = any amino acid) | |
| α chain w/WT signal | MLLLLIPVLGMIFALRDARAQSVSQHNHHVILSEAASLELGCNYSYGGTVNL | 1178 |
| peptide, Cα | FWYVQYPGQHLQLLLKYFSGDPLVKGIKGFEAEFIKSKFSFNLRKPSVQWSD | |
| (substituted) | TAEYFCAVKGDYKLSFGAGTTVTVRANIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| α chain w/alternative | MALLLIPVLGMIFALRDARAQSVSQHNHHVILSEAASLELGCNYSYGGTVNL | 1179 |
| signal peptide, Cα | FWYVQYPGQHLQLLLKYFSGDPLVKGIKGFEAEFIKSKFSFNLRKPSVQWSD | |
| (substituted) | TAEYFCAVKGDYKLSFGAGTTVTVRANIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| α chain w/alternative | MHLLLIPVLGMIFALRDARAQSVSQHNHHVILSEAASLELGCNYSYGGTVNL | 1180 |
| signal peptide, Cα | FWYVQYPGQHLQLLLKYFSGDPLVKGIKGFEAEFIKSKESENLRKPSVQWSD | |
| (substituted) | TAEYFCAVKGDYKLSFGAGTTVTVRANIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| CDR1β | SGHAT | 2171 |
| CDR2β | FQNNGV | 2172 |
| CDR3β | ASSLVNTEAF | 2173 |
| Vβ w/o signal peptide | GVAQSPRYKIIEKRQSVAFWCNPISGHATLYWYQQILGQGPKLLIQFQNNGV | 2174 |
| (SignalP) | VDDSQLPKDRFSAERLKGVDSTLKIQPAKLEDSAVYLCASSLVNTEAFFGQG | |
| | TRLTVV | |
| Vβ w/o signal peptide | EAGVAQSPRYKIIEKRQSVAFWCNPISGHATLYWYQQILGQGPKLLIQFQNN | 2175 |
| (IMGT) | GVVDDSQLPKDRFSAERLKGVDSTLKIQPAKLEDSAVYLCASSLVNTEAFFG | |
| | QGTRLTVV | |
| Vβ | MXTRLLCWAALCLLGAELTEAGVAQSPRYKIIEKRQSVAFWCNPISGHATLY | 2176 |
| | WYQQILGQGPKLLIQFQNNGVVDDSQLPKDRFSAERLKGVDSTLKIQPAKLE | 2177 |
| | DSAVYLCASSLVNTEAFFGQGTRLTVV | |
| | (X = any amino acid) | |
| β chain w/WT signal | MGTRLLCWAALCLLGAELTEAGVAQSPRYKIIEKRQSVAFWCNPISGHATLY | 2178 |
| peptide, Cβ | WYQQILGQGPKLLIQFQNNGVVDDSQLPKDRFSAERLKGVDSTLKIQPAKLE | |
| (substituted) | DSAVYLCASSLVNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain w/alternative | MATRLLCWAALCLLGAELTEAGVAQSPRYKIIEKRQSVAFWCNPISGHATLY | 2179 |
| signal peptide, Cβ | WYQQILGQGPKLLIQFQNNGVVDDSQLPKDRFSAERLKGVDSTLKIQPAKLE | |
| (substituted) | DSAVYLCASSLVNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain w/alternative | MHTRLLCWAALCLLGAELTEAGVAQSPRYKIIEKRQSVAFWCNPISGHATLY | 2180 |
| signal peptide, Cβ | WYQQILGQGPKLLIQFQNNGVVDDSQLPKDRFSAERLKGVDSTLKIQPAKLE | |
| (substituted) | DSAVYLCASSLVNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR018 interacts with and/or is specific for p53. In some embodiments, the peptide is from a neoantigen of p53. In some embodiments, the neoantigen has the amino acid change G245S relative to the wild type p53 sequence. In some embodiments, TCR018 interacts with the neoantigen in the context of HLA-DRB3*02:02, as described in

TABLE 6S
Amino acid sequences of TCR019.

| | | SEQ |
|---|---|---|
| | | ID |
| Description | Sequence | NO: |
| CDR1α | TRDTTYY | 1181 |
| CDR2α | RNSFDEQN | 1182 |
| CDR3α | ALSEGGSNYKLT | 1183 |
| Vα w/o signal peptide | QKVTQAQTEISVVEKEDVTLDCVYETRDTTYYLFWYKQPPSGELVFLIRRNS | 1184 |
| (SignalP) | FDEQNEISGRYSWNFQKSTSSENFTITASQVVDSAVYFCALSEGGSNYKLTF | |
| | GKGTLLTVNP | |
| Vα w/o signal peptide | AQKVTQAQTEISVVEKEDVTLDCVYETRDTTYYLFWYKQPPSGELVFLIRRN | 1185 |
| (IMGT) | SFDEQNEISGRYSWNFQKSTSSENFTITASQVVDSAVYFCALSEGGSNYKLT | |
| | FGKGTLLTVNP | |
| Vα | MXTASLLRAVIASICVVSSMAQKVTQAQTEISVVEKEDVTLDCVYETRDTTY | 1186 |
| | YLFWYKQPPSGELVFLIRRNSFDEQNEISGRYSWNFQKSTSSENFTITASQV | 1187 |
| | VDSAVYFCALSEGGSNYKLTFGKGTLLTVNP | |
| | (X = any amino acid) | |
| α chain w/WT signal | MLTASLLRAVIASICVVSSMAQKVTQAQTEISVVEKEDVTLDCVYETRDTTY | 1188 |
| peptide, Cα | YLFWYKQPPSGELVFLIRRNSFDEQNEISGRYSWNFQKSTSSENFTITASQV | |
| (substituted) | VDSAVYFCALSEGGSNYKLTFGKGTLLTVNPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain w/alternative | MATASLLRAVIASICVVSSMAQKVTQAQTEISVVEKEDVTLDCVYETRDTTY | 1189 |
| signal peptide, Cα | YLFWYKQPPSGELVFLIRRNSFDEQNEISGRYSWNFQKSTSSENFTITASQV | |
| (substituted) | VDSAVYFCALSEGGSNYKLTFGKGTLLTVNPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain w/alternative | MHTASLLRAVIASICVVSSMAQKVTQAQTEISVVEKEDVTLDCVYETRDTTY | 1190 |
| signal peptide, Cα | YLFWYKQPPSGELVFLIRRNSFDEQNEISGRYSWNFQKSTSSENFTITASQV | |
| (substituted) | VDSAVYFCALSEGGSNYKLTFGKGTLLTVNPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| CDR1β | LNHNV | 2181 |
| CDR2β | YYDKDF | 2182 |
| CDR3β | ATSRELRGNEQF | 2183 |
| Vβ w/o signal peptide | DAMVIQNPRYQVTQFGKPVTLSCSQTLNHNVMYWYQQKSSQAPKLLFHYYDK | 2184 |
| (SignalP) | DENNEADTPDNFQSRRPNTSFCFLDIRSPGLGDAAMYLCATSRELRGNEQFF | |
| | GPGTRLTVL | |
| Vβ w/o signal peptide | DAMVIQNPRYQVTQFGKPVTLSCSQTLNHNVMYWYQQKSSQAPKLLFHYYDK | 2185 |
| (IMGT) | DENNEADTPDNFQSRRPNTSFCFLDIRSPGLGDAAMYLCATSRELRGNEQFF | |
| | GPGTRLTVL | |
| Vβ | MXPGLLHWMALCLLGTGHGDAMVIQNPRYQVTQFGKPVTLSCSQTLNHNVMY | 2186 |
| | WYQQKSSQAPKLLFHYYDKDENNEADTPDNFQSRRPNTSFCFLDIRSPGLGD | 2187 |
| | AAMYLCATSRELRGNEQFFGPGTRLTVL | |
| | (X = any amino acid) | |
| β chain w/WT signal | MGPGLLHWMALCLLGTGHGDAMVIQNPRYQVTQFGKPVTLSCSQTLNHNVMY | 2188 |
| peptide, Cβ | WYQQKSSQAPKLLFHYYDKDENNEADTPDNFQSRRPNTSFCFLDIRSPGLGD | |
| (substituted) | AAMYLCATSRELRGNEQFFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAPGLLHWMALCLLGTGHGDAMVIQNPRYQVTQFGKPVTLSCSQTLNHNVMY | 2189 |
| signal peptide, Cf | WYQQKSSQAPKLLFHYYDKDENNEADTPDNFQSRRPNTSFCFLDIRSPGLGD | |
| (substituted) | AAMYLCATSRELRGNEQFFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHPGLLHWMALCLLGTGHGDAMVIQNPRYQVTQFGKPVTLSCSQTLNHNVMY | 2190 |
| signal peptide, Cβ | WYQQKSSQAPKLLFHYYDKDENNEADTPDNFQSRRPNTSFCFLDIRSPGLGD | |
| (substituted) | AAMYLCATSRELRGNEQFFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR019 interacts with and/or is specific for p53. In some embodiments, the peptide is from a neoantigen of p53. In some embodiments, the neoantigen has the amino acid change G245S relative to the wild type p53 sequence. In some embodiments, TCR019 interacts with the neoantigen in the context of HLA-DRB3*02:02, as described in

TABLE 6T
Amino acid sequences of TCR020.

| | | SEQ |
|---|---|---|
| | | ID |
| Description | Sequence | NO: |
| CDR1α | DRGSQS | 1191 |
| CDR2α | IYSNGD | 1192 |
| CDR3α | AVNDAGNMLT | 1193 |
| Vα without signal | QQKEVEQNSGPLSVPEGAIASLNCTYSDRGSQSFFWYRQYSGKSPELIMFIY | 1194 |
| peptide (SignalP) | SNGDKEDGRFTAQLNKASQYVSLLIRDSQPSDSATYLCAVNDAGNMLTFGGG | |
| | TRLMVKP | |
| Vα without signal | QKEVEQNSGPLSVPEGAIASLNCTYSDRGSQSFFWYRQYSGKSPELIMFIYS | 1195 |
| peptide (IMGT) | NGDKEDGRFTAQLNKASQYVSLLIRDSQPSDSATYLCAVNDAGNMLTFGGGT | |
| | RLMVKP | |
| Vα | MXSLRVLLVILWLQLSWVWSQQKEVEQNSGPLSVPEGAIASLNCTYSDRGSQ | 1196 |
| | SFFWYRQYSGKSPELIMFIYSNGDKEDGRFTAQLNKASQYVSLLIRDSQPSD | 1197 |
| | SATYLCAVNDAGNMLTFGGGTRLMVKP | |
| | (X = any amino acid) | |
| α chain with WT signal | MKSLRVLLVILWLQLSWVWSQQKEVEQNSGPLSVPEGAIASLNCTYSDRGSQ | 1198 |
| peptide, Cα | SFFWYRQYSGKSPELIMFIYSNGDKEDGRETAQLNKASQYVSLLIRDSQPSD | |
| (substituted) | SATYLCAVNDAGNMLTFGGGTRLMVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain with | MASLRVLLVILWLQLSWVWSQQKEVEQNSGPLSVPEGAIASLNCTYSDRGSQ | 1199 |
| alternative signal | SFFWYRQYSGKSPELIMFIYSNGDKEDGRFTAQLNKASQYVSLLIRDSQPSD | |
| peptide, Cα | SATYLCAVNDAGNMLTFGGGTRLMVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| 8 | MHSLRVLLVILWLQLSWVWSQQKEVEQNSGPLSVPEGAIASLNCTYSDRGSQ | 1200 |
| chain with | SFFWYRQYSGKSPELIMFIYSNGDKEDGRFTAQLNKASQYVSLLIRDSQPSD | |
| alternative signal | SATYLCAVNDAGNMLTFGGGTRLMVKPNIQNPEPAVYQLKDPRSQDSTLCLF | |
| peptide, Cα | TDFDSQINVPKTMESGTFITDKCVLDMKAMDSKSNGAIAWSNQTSFTCQDIF | |
| (substituted) | KETNATYPSSDVPCDATLTEKSFETDMNLNFQNLLVIVLRILLLKVAGENLL | |
| | MTLRLWSS | |
| CDR1β | DFQATT | 2191 |
| CDR2β | SNEGSKA | 2192 |
| CDR3β | SAAGQANTEAF | 2193 |
| Vβ without signal | GSGLGAVVSQHPSWVICKSGTSVKIECRSLDFQATTMFWYRQFPKQSLMLMA | 2194 |
| peptide (SignalP) | TSNEGSKATYEQGVEKDKFLINHASLTLSTLTVTSAHPEDSSFYICSAAGQA | |
| | NTEAFFGQGTRLTVV | |
| Vβ without signal | GAVVSQHPSWVICKSGTSVKIECRSLDFQATTMFWYRQFPKQSLMLMATSNE | 2195 |
| peptide (IMGT) | GSKATYEQGVEKDKFLINHASLTLSTLTVTSAHPEDSSFYICSAAGQANTEA | |
| | FFGQGTRLTVV | |
| Vβ | MXLLLLLLGPGISLLLPGSLAGSGLGAVVSQHPSWVICKSGTSVKIECRSLD | 2196 |
| | FQATTMFWYRQFPKQSLMLMATSNEGSKATYEQGVEKDKFLINHASLTLSTL | 2197 |
| | TVTSAHPEDSSFYICSAAGQANTEAFFGQGTRLTVV | |
| | (X = any amino acid) | |
| β chain with WT signal | MLLLLLLLGPGISLLLPGSLAGSGLGAVVSQHPSWVICKSGTSVKIECRSLD | 2198 |
| peptide, Cβ | FQATTMFWYRQFPKQSLMLMATSNEGSKATYEQGVEKDKFLINHASLTLSTL | |
| (substituted) | TVTSAHPEDSSFYICSAAGQANTEAFFGQGTRLTVVEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MALLLLLLGPGISLLLPGSLAGSGLGAVVSQHPSWVICKSGTSVKIECRSLD | 2199 |
| signal peptide, Cβ | FQATTMFWYRQFPKQSLMLMATSNEGSKATYEQGVEKDKFLINHASLTLSTL | |
| (substituted) | TVTSAHPEDSSFYICSAAGQANTEAFFGQGTRLTVVEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MHLLLLLLGPGISLLLPGSLAGSGLGAVVSQHPSWVICKSGTSVKIECRSLD | 2200 |
| signal peptide, Cβ | FQATTMFWYRQFPKQSLMLMATSNEGSKATYEQGVEKDKFLINHASLTLSTL | |
| (substituted) | TVTSAHPEDSSFYICSAAGQANTEAFFGQGTRLTVVEDLRNVTPPKVSLEEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |

In some embodiments, TCR020 interacts with and/or is specific for p53. In some embodiments, the peptide is from a neoantigen of p53. In some embodiments, the neoantigen has the amino acid change G245S relative to the wild type p53 sequence. In some embodiments, TCR020 interacts with the neoantigen in the context of HLA-DRB3*02:02, as described in International Publication No. WO 2019/067243, incorporated herein by reference in its entirety.

TABLE 6U
Amino acid sequences of TCR021.

| | | SEQ |
|---|---|---|
| | | ID |
| Description | Sequence | NO: |
| CDR1α | TRDTTYY | 1201 |
| CDR2α | RNSFDEQN | 1202 |
| CDR3α | ALSEVDSGNTPLV | 1203 |
| Vα without signal | QKVTQAQTEISVVEKEDVTLDCVYETRDTTYYLFWYKQPPSGELVFLIRRNS | 1204 |
| peptide (SignalP) | FDEQNEISGRYSWNFQKSTSSFNFTITASQVVDSAVYFCALSEVDSGNTPLV | |
| | FGKGTRLSVIA | |
| Vα without signal | AQKVTQAQTEISVVEKEDVTLDCVYETRDTTYYLFWYKQPPSGELVFLIRRN | 1205 |
| peptide (IMGT) | SFDEQNEISGRYSWNFQKSTSSENFTITASQVVDSAVYFCALSEVDSGNTPL | |
| | VFGKGTRLSVIA | |
| Vα | MXTASLLRAVIASICVVSSMAQKVTQAQTEISVVEKEDVTLDCVYETRDTTY | 1206 |
| | YLFWYKQPPSGELVFLIRRNSFDEQNEISGRYSWNFQKSTSSENFTITASQV | 1207 |
| | VDSAVYFCALSEVDSGNTPLVFGKGTRLSVIA | |
| | (X = any amino acid) | |
| α chain with WT signal | MLTASLLRAVIASICVVSSMAQKVTQAQTEISVVEKEDVTLDCVYETRDTTY | 1208 |
| peptide, Cα | YLFWYKQPPSGELVFLIRRNSFDEQNEISGRYSWNFQKSTSSENFTITASQV | |
| (substituted) | VDSAVYFCALSEVDSGNTPLVFGKGTRLSVIANIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α | MATASLLRAVIASICVVSSMAQKVTQAQTEISVVEKEDVTLDCVYETRDTTY | 1209 |
| chain with | YLFWYKQPPSGELVFLIRRNSFDEQNEISGRYSWNFQKSTSSENFTITASQV | |
| alternative signal | VDSAVYFCALSEVDSGNTPLVFGKGTRLSVIANIQNPEPAVYQLKDPRSQDS | |
| peptide, Cα | TLCLFTDFDSQINVPKTMESGTFITDKCVLDMKAMDSKSNGAIAWSNQTSFT | |
| (substituted) | CQDIFKETNATYPSSDVPCDATLTEKSFETDMNLNFQNLLVIVLRILLLKVA | |
| | GFNLLMTLRLWSS | |
| α | MHTASLLRAVIASICVVSSMAQKVTQAQTEISVVEKEDVTLDCVYETRDTTY | 1210 |
| chain with | YLFWYKQPPSGELVFLIRRNSFDEQNEISGRYSWNFQKSTSSENFTITASQV | |
| alternative signal | VDSAVYFCALSEVDSGNTPLVFGKGTRLSVIANIQNPEPAVYQLKDPRSQDS | |
| peptide, Cα | TLCLFTDFDSQINVPKTMESGTFITDKCVLDMKAMDSKSNGAIAWSNQTSFT | |
| (substituted) | CQDIFKETNATYPSSDVPCDATLTEKSFETDMNLNFQNLLVIVLRILLLKVA | |
| | GFNLLMTLRLWSS | |
| CDR1β | SGDLS | 2201 |
| CDR2β | YYNGEE | 2202 |
| CDR3β | ASSVGSSSSTDTQY | 2203 |
| VB without signal | GVTQTPKHLITATGQRVTLRCSPRSGDLSVYWYQQSLDQGLQFLIQYYNGEE | 2204 |
| peptide (SignalP) | RAKGNILERFSAQQFPDLHSELNLSSLELGDSALYFCASSVGSSSSTDTQYF | |
| | GPGTRLTVL | |
| Vβ without signal | DSGVTQTPKHLITATGQRVTLRCSPRSGDLSVYWYQQSLDQGLQFLIQYYNG | 2205 |
| peptide (IMGT) | EERAKGNILERFSAQQFPDLHSELNLSSLELGDSALYFCASSVGSSSSTDTQ | |
| | YFGPGTRLTVL | |
| Vβ | MXHFRLLCCVAFCLLGAGPVDSGVTQTPKHLITATGQRVTLRCSPRSGDLSV | 2206 |
| | YWYQQSLDQGLQFLIQYYNGEERAKGNILERFSAQQFPDLHSELNLSSLELG | 2207 |
| | DSALYFCASSVGSSSSTDTQYFGPGTRLTVL | |
| | (X = any amino acid) | |
| β chain with WT signal | MGFRLLCCVAFCLLGAGPVDSGVTQTPKHLITATGQRVTLRCSPRSGDLSVY | 2208 |
| peptide, Cβ | WYQQSLDQGLQFLIQYYNGEERAKGNILERFSAQQFPDLHSELNLSSLELGD | |
| (substituted) | SALYFCASSVGSSSSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MAFRLLCCVAFCLLGAGPVDSGVTQTPKHLITATGQRVTLRCSPRSGDLSVY | 2209 |
| signal peptide, Cβ | WYQQSLDQGLQFLIQYYNGEERAKGNILERFSAQQFPDLHSELNLSSLELGD | |
| (substituted) | SALYFCASSVGSSSSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHFRLLCCVAFCLLGAGPVDSGVTQTPKHLITATGQRVTLRCSPRSGDLSVY | 2210 |
| signal peptide, Cβ | WYQQSLDQGLQFLIQYYNGEERAKGNILERFSAQQFPDLHSELNLSSLELGD | |
| (substituted) | SALYFCASSVGSSSSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR021 interacts with and/or is specific for p53. In some embodiments, the peptide is from a neoantigen of p53. In some embodiments, the neoantigen has the amino acid change R248Q relative to the wild type p53 sequence. In some embodiments, TCR021 interacts with the neoantigen in the context of HLA-A*02:01, as described in International Publication No. WO 2019/067243, incorporated herein by reference in its entirety.

TABLE 6V
Amino acid sequences of TCR022.

| | | SEQ |
|---|---|---|
| | | ID |
| Description | Sequence | NO: |
| CDR1α | NTAFDY | 1771 |
| CDR2α | IRPDVSE | 1772 |
| CDR3α | AAEAGNHRGSTLGRLY | 1773 |
| Vα w/o signal peptide | QQKEKSDQQQVKQSPQSLIVQKGGISIINCAYENTAFDYFPWYQQFPGKGPA | 1774 |
| (SignalP) | LLIAIRPDVSEKKEGRFTISFNKSAKQFSLHIMDSQPGDSATYFCAAEAGNH | |
| | RGSTLGRLYFGRGTQLTVWP | |
| Vα w/o signal peptide | QQQVKQSPQSLIVQKGGISIINCAYENTAFDYFPWYQQFPGKGPALLIAIRP | 1775 |
| (IMGT) | DVSEKKEGRFTISFNKSAKQFSLHIMDSQPGDSATYFCAAEAGNHRGSTLGR | |
| | LYFGRGTQLTVWP | |
| Vα | MXKILGASFLVLWLQLCWVSGQQKEKSDQQQVKQSPQSLIVQKGGISIINCA | 1776 |
| | YENTAFDYFPWYQQFPGKGPALLIAIRPDVSEKKEGRFTISFNKSAKQFSLH | 1777 |
| | IMDSQPGDSATYFCAAEAGNHRGSTLGRLYFGRGTQLTVWP | |
| | (X = any amino acid) | |
| α chain with WT signal | MDKILGASFLVLWLQLCWVSGQQKEKSDQQQVKQSPQSLIVQKGGISIINCA | 1778 |
| peptide, Cα | YENTAFDYFPWYQQFPGKGPALLIAIRPDVSEKKEGRFTISENKSAKQFSLH | |
| (substituted) | IMDSQPGDSATYFCAAEAGNHRGSTLGRLYFGRGTQLTVWPNIQNPEPAVYQ | |
| | LKDPRSQDSTLCLFTDFDSQINVPKTMESGTFITDKCVLDMKAMDSKSNGAI | |
| | AWSNQTSFTCQDIFKETNATYPSSDVPCDATLTEKSFETDMNLNFQNLLVIV | |
| | LRILLLKVAGENLLMTLRLWSS | |
| α | MAKILGASFLVLWLQLCWVSGQQKEKSDQQQVKQSPQSLIVQKGGISIINCA | 1779 |
| chain with | YENTAFDYFPWYQQFPGKGPALLIAIRPDVSEKKEGRFTISENKSAKQFSLH | |
| alternative signal | IMDSQPGDSATYFCAAEAGNHRGSTLGRLYFGRGTQLTVWPNIQNPEPAVYQ | |
| peptide, Cα | LKDPRSQDSTLCLFTDFDSQINVPKTMESGTFITDKCVLDMKAMDSKSNGAI | |
| (substituted) | AWSNQTSFTCQDIFKETNATYPSSDVPCDATLTEKSFETDMNLNFQNLLVIV | |
| | LRILLLKVAGENLLMTLRLWSS | |
| α | MHKILGASFLVLWLQLCWVSGQQKEKSDQQQVKQSPQSLIVQKGGISIINCA | 1780 |
| chain with | YENTAFDYFPWYQQFPGKGPALLIAIRPDVSEKKEGRFTISFNKSAKQFSLH | |
| alternative signal | IMDSQPGDSATYFCAAEAGNHRGSTLGRLYFGRGTQLTVWPNIQNPEPAVYQ | |
| peptide, Cα | LKDPRSQDSTLCLFTDFDSQINVPKTMESGTFITDKCVLDMKAMDSKSNGAI | |
| (substituted) | AWSNQTSFTCQDIFKETNATYPSSDVPCDATLTEKSFETDMNLNFQNLLVIV | |
| | LRILLLKVAGENLLMTLRLWSS | |
| CDR1β | SGHRS | 2771 |
| CDR2β | YFSETQ | 2772 |
| CDR3β | ASSLAAGGYFNEQF | 2773 |
| Vβ w/o signal peptide | GVTQTPRYLIKTRGQQVTLSCSPISGHRSVSWYQQTPGQGLQFLFEYESETQ | 2774 |
| (SignalP) | RNKGNFPGRFSGRQFSNSRSEMNVSTLELGDSALYLCASSLAAGGYFNEQFF | |
| | GPGTRLTVL | |
| Vβ w/o signal peptide | KAGVTQTPRYLIKTRGQQVTLSCSPISGHRSVSWYQQTPGQGLQFLFEYFSE | 2775 |
| (IMGT) | TQRNKGNFPGRFSGRQFSNSRSEMNVSTLELGDSALYLCASSLAAGGYFNEQ | |
| | FFGPGTRLTVL | |
| Vβ | MXSRLLCWVLLCLLGAGPVKAGVTQTPRYLIKTRGQQVTLSCSPISGHRSVS | 2776 |
| | WYQQTPGQGLQFLFEYFSETQRNKGNFPGRESGRQFSNSRSEMNVSTLELGD | 2777 |
| | SALYLCASSLAAGGYFNEQFFGPGTRLTVL | |
| | (X = any amino acid) | |
| β chain with WT signal | MGSRLLCWVLLCLLGAGPVKAGVTQTPRYLIKTRGQQVTLSCSPISGHRSVS | 2778 |
| peptide, Cβ | WYQQTPGQGLQFLFEYFSETQRNKGNFPGRFSGRQFSNSRSEMNVSTLELGD | |
| (substituted) | SALYLCASSLAAGGYFNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MASRLLCWVLLCLLGAGPVKAGVTQTPRYLIKTRGQQVTLSCSPISGHRSVS | 2779 |
| signal peptide, Cβ | WYQQTPGQGLQFLFEYFSETQRNKGNFPGRFSGRQFSNSRSEMNVSTLELGD | |
| (substituted) | SALYLCASSLAAGGYFNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHSRLLCWVLLCLLGAGPVKAGVTQTPRYLIKTRGQQVTLSCSPISGHRSVS | 2780 |
| signal peptide, Cβ | WYQQTPGQGLQFLFEYFSETQRNKGNFPGRESGRQFSNSRSEMNVSTLELGD | |
| (substituted) | SALYLCASSLAAGGYFNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR022 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR022 interacts with the neoantigen in the context of HLA-A*11:01, as described in International Publication No. WO 2021/163434, incorporated herein by reference in its entirety.

TABLE 6W
Amino acid sequences of TCR023.

| | | SEQ |
|---|---|---|
| Descrip- | | ID |
| tion | Sequence | NO: |
| CDR1α | NSAFQY | 1221 |
| CDR2α | TYSSGN | 1222 |
| CDR3α | AMTSPYNNNDMR | 1223 |
| Vα | QQKEVEQDPGPLSVPEGAIVSLNCTYSNSAFQY | 1224 |
| without | FMWYRQYSRKGPELLMYTYSSGNKEDGRFTAQV | |
| signal | DKSSKYISLFIRDSQPSDSATYLCAMTSPYNNN | |
| peptide | DMRFGAGTRLTVKP | |
| (SignalP) | | |
| Vα | QKEVEQDPGPLSVPEGAIVSLNCTYSNSAFQYF | 1225 |
| without | MWYRQYSRKGPELLMYTYSSGNKEDGRFTAQVD | |
| signal | KSSKYISLFIRDSQPSDSATYLCAMTSPYNNND | |
| peptide | MRFGAGTRLTVKP | |
| (IMGT) | | |
| Vα | MXKSLRVLLVILWLQLSWVWSQQKEVEQDPGPL | 1226 |
| | SVPEGAIVSINCTYSNSAFQYFMWYRQYSRKGP | |
| | ELLMYTYSSGNKEDGRFTAQVDKSSKYISLFIR | |
| | DSQPSDSATYLCAMTSPYNNNDMRFGAGTRLTV | |
| | KP | |
| | (X = any amino acid) | 1227 |
| α chain | MCKSLRVLLVILWLQLSWVWSQQKEVEQDPGPL | 1228 |
| with WT | SVPEGAIVSLNCTYSNSAFQYFMWYRQYSRKGP | |
| signal | ELLMYTYSSGNKEDGRFTAQVDKSSKYISLFIR | |
| peptide, | DSQPSDSATYLCAMTSPYNNNDMRFGAGTRLTV | |
| Cα (sub- | KPNIQNPEPAVYQLKDPRSQDSTLCLFTDFDSQ | |
| stituted) | INVPKTMESGTFITDKCVLDMKAMDSKSNGAIA | |
| | WSNQTSFTCQDIFKETNATYPSSDVPCDATLTE | |
| | KSFETDMNLNFQNLLVIVLRILLLKVAGFNLLM | |
| | TLRLWSS | |
| α chain | MAKSLRVLLVILWLQLSWVWSQQKEVEQDPGPL | 1229 |
| with al- | SVPEGAIVSLNCTYSNSAFQYFMWYRQYSRKGP | |
| ternative | ELLMYTYSSGNKEDGRFTAQVDKSSKYISLFIR | |
| signal | DSQPSDSATYLCAMTSPYNNNDMRFGAGTRLTV | |
| peptide, | KPNIQNPEPAVYQLKDPRSQDSTLCLFTDFDSQ | |
| Cα (sub- | INVPKTMESGTFITDKCVLDMKAMDSKSNGAIA | |
| stituted) | WSNQTSFTCQDIFKETNATYPSSDVPCDATLTE | |
| | KSFETDMNLNFQNLLVIVLRILLLKVAGFNLLM | |
| | TLRLWSS | |
| α chain | MHKSLRVLLVILWLQLSWVWSQQKEVEQDPGPL | 1230 |
| with al- | SVPEGAIVSLNCTYSNSAFQYFMWYRQYSRKGP | |
| ternative | ELLMYTYSSGNKEDGRFTAQVDKSSKYISLFIR | |
| signal | DSQPSDSATYLCAMTSPYNNNDMRFGAGTRLTV | |
| peptide, | KPNIQNPEPAVYQLKDPRSQDSTLCLFTDFDSQ | |
| Cα (sub- | INVPKTMESGTFITDKCVLDMKAMDSKSNGAIA | |
| stituted) | WSNQTSFTCQDIFKETNATYPSSDVPCDATLTE | |
| | KSFETDMNLNFQNLLVIVLRILLLKVAGFNLLM | |
| | TLRLWSS | |
| CDR1ß | DFQATT | 2221 |
| CDR2ß | SNEGSKA | 2222 |
| CDR3ß | SGGLEEAARQFI | 2223 |
| Vß | GSGLGAVVSQHPSWVICKSGTSVKIECRSLDFQ | 2224 |
| without | ATTMFWYRQFPKQSLMLMATSNEGSKATYEQGV | |
| signal | EKDKFLINHASLTLSTLTVTSAHPEDSSFYICS | |
| peptide | GGLEEAARQFIGPGTRLTVL | |
| (SignalP) | | |
| Vß | GAVVSQHPSWVICKSGTSVKIECRSLDFQATTM | 2225 |
| without | FWYRQFPKQSLMLMATSNEGSKATYEQGVEKDK | |
| signal | FLINHASLTLSTLTVTSAHPEDSSFYICSGGLE | |
| peptide | EAARQFIGPGTRLTVL | |
| (IMGT) | | |
| Vß | MXLLLLLLGPGISLLLPGSLAGSGLGAVVSQHP | 2226 |
| | SWVICKSGTSVKIECRSLDFQATTMFWYRQFPK | |
| | QSLMLMATSNEGSKATYEQGVEKDKFLINHASL | |
| | TLSTLTVTSAHPEDSSFYICSGGLEEAARQFIG | |
| | PGTRLTVL | |
| | (X = any amino acid) | 2227 |
| ß chain | MLLLLLLLGPGISLLLPGSLAGSGLGAVVSQHP | 2228 |
| with WT | SWVICKSGTSVKIECRSLDFQATTMFWYRQFPK | |
| signal | QSLMLMATSNEGSKATYEQGVEKDKFLINHASL | |
| peptide, | TLSTLTVTSAHPEDSSFYICSGGLEEAARQFIG | |
| Cß (sub- | PGTRLTVLEDLRNVTPPKVSLFEPSKAEIANKQ | |
| stituted) | KATLVCLARGFFPDHVELSWWVNGKEVHSGVCT | |
| | DPQAYKESNYSYCLSSRLRVSATFWHNPRNHFR | |
| | CQVQFHGLSEEDKWPEGSPKPVTQNISAEAWGR | |
| | ADCGITSASYQQGVLSATILYEILLGKATLYAV | |
| | LVSTLVVMAMVKRKNS | |
| ß chain | MALLLLLLGPGISLLLPGSLAGSGLGAVVSQHP | 2229 |
| with al- | SWVICKSGTSVKIECRSLDFQATTMFWYRQFPK | |
| ternative | QSLMLMATSNEGSKATYEQGVEKDKFLINHASL | |
| signal | TLSTLTVTSAHPEDSSFYICSGGLEEAARQFIG | |
| peptide, | PGTRLTVLEDLRNVTPPKVSLFEPSKAEIANKQ | |
| Cß (sub- | KATLVCLARGFFPDHVELSWWVNGKEVHSGVCT | |
| stituted) | DPQAYKESNYSYCLSSRLRVSATFWHNPRNHFR | |
| | CQVQFHGLSEEDKWPEGSPKPVTQNISAEAWGR | |
| | ADCGITSASYQQGVLSATILYEILLGKATLYAV | |
| | LVSTLVVMAMVKRKNS | |
| ß chain | MHLLLLLLGPGISLLLPGSLAGSGLGAVVSQHP | 2230 |
| with al- | SWVICKSGTSVKIECRSLDFQATTMFWYRQFPK | |
| ternative | QSLMLMATSNEGSKATYEQGVEKDKFLINHASL | |
| signal | TLSTLTVTSAHPEDSSFYICSGGLEEAARQFIG | |
| peptide, | PGTRLTVLEDLRNVTPPKVSLFEPSKAEIANKQ | |
| Cß (sub- | KATLVCLARGFFPDHVELSWWVNGKEVHSGVCT | |
| stituted) | DPQAYKESNYSYCLSSRLRVSATFWHNPRNHFR | |
| | CQVQFHGLSEEDKWPEGSPKPVTQNISAEAWGR | |
| | ADCGITSASYQQGVLSATILYEILLGKATLYAV | |
| | LVSTLVVMAMVKRKNS | |

In some embodiments, TCR023 interacts with and/or is specific for p53. In some embodiments, the peptide is from a neoantigen of p53. In some embodiments, the neoantigen has the amino acid change R248Q relative to the wild type p53 sequence. In some embodiments, TCR023 interacts with the neoantigen in the context of HLA-A*02:01, as described in

TABLE 6X
Amino acid sequences of TCR024.

| | | SEQ |
|---|---|---|
| Descrip- | | ID |
| tion | Sequence | NO: |
| CDR1α | NSAFQY | 1231 |
| CDR2α | TYSSGN | 1232 |
| CDR3α | AMTSPYNNNDMR | 1233 |
| Vα | QQKEVEQDPGPLSVPEGAIVSLNCTYSNSAFQY | 1234 |
| without | FMWYRQYSRKGPELLMYTYSSGNKEDGRFTAQV | |
| signal | DKSSKYISLFIRDSQPSDSATYLCAMTSPYNNN | |
| peptide | DMRFGAGTRLTVKP | |
| (SignalP) | | |
| Vα | QKEVEQDPGPLSVPEGAIVSLNCTYSNSAFQYF | 1235 |
| without | MWYRQYSRKGPELLMYTYSSGNKEDGRFTAQVD | |
| signal | KSSKYISLFIRDSQPSDSATYLCAMTSPYNNND | |
| peptide | MRFGAGTRLTVKP | |
| (IMGT) | | |
| Vα | MXKSLRVLLVILWLQLSWVWSQQKEVEQDPGPL | 1236 |
| | SVPEGAIVSLNCTYSNSAFQYFMWYRQYSRKGP | |
| | ELLMYTYSSGNKEDGRFTAQVDKSSKYISLFIR | |
| | DSQPSDSATYLCAMTSPYNNNDMRFGAGTRLTV | |
| | KP | |
| | (X = any amino acid) | 1237 |
| α chain | MCKSLRVLLVILWLQLSWVWSQQKEVEQDPGPL | 1238 |
| with WT | SVPEGAIVSLNCTYSNSAFQYFMWYRQYSRKGP | |
| signal | ELLMYTYSSGNKEDGRFTAQVDKSSKYISLFIR | |
| peptide, | DSQPSDSATYLCAMTSPYNNNDMRFGAGTRLTV | |
| Cα (sub- | KPNIQNPEPAVYQLKDPRSQDSTLCLFTDFDSQ | |
| stituted) | INVPKTMESGTFITDKCVLDMKAMDSKSNGAIA | |
| | WSNQTSFTCQDIFKETNATYPSSDVPCDATLTE | |
| | KSFETDMNLNFQNLLVIVLRILLLKVAGFNLLM | |
| | TLRLWSS | |
| α chain | MAKSLRVLLVILWLQLSWVWSQQKEVEQDPGPL | 1239 |
| with al- | SVPEGAIVSLNCTYSNSAFQYFMWYRQYSRKGP | |
| ternative | ELLMYTYSSGNKEDGRFTAQVDKSSKYISLFIR | |
| signal | DSQPSDSATYLCAMTSPYNNNDMRFGAGTRLTV | |
| peptide, | KPNIQNPEPAVYQLKDPRSQDSTLCLFTDFDSQ | |
| Cα (subs- | INVPKTMESGTFITDKCVLDMKAMDSKSNGAIA | |
| tituted) | WSNQTSFTCQDIFKETNATYPSSDVPCDATLTE | |
| | KSFETDMNLNFQNLLVIVLRILLLKVAGFNLLM | |
| | TLRLWSS | |
| α chain | MHKSLRVLLVILWLQLSWVWSQQKEVEQDPGPL | 1240 |
| with al- | SVPEGAIVSLNCTYSNSAFQYFMWYRQYSRKGP | |
| ternative | ELLMYTYSSGNKEDGRFTAQVDKSSKYISLFIR | |
| signal | DSQPSDSATYLCAMTSPYNNNDMRFGAGTRLTV | |
| peptide, | KPNIQNPEPAVYQLKDPRSQDSTLCLFTDFDSQ | |
| Cα (sub- | INVPKTMESGTFITDKCVLDMKAMDSKSNGAIA | |
| stituted) | WSNQTSFTCQDIFKETNATYPSSDVPCDATLTE | |
| | KSFETDMNLNFQNLLVIVLRILLLKVAGFNLLM | |
| | TLRLWSS | |
| CDR1ß | DFQATT | 2231 |
| CDR2ß | SNEGSKA | 2232 |
| CDR3ß | SGGLEEAARQFI | 2233 |
| Vß | AVVSQHPSRVICKSGTSVKIECRSLDFQATTMF | 2234 |
| without | WYRQFPKQSLMLMATSNEGSKATYEQGVEKDKF | |
| signal | LINHASLTLSTLTVTSAHPEDSSFYICSGGLEE | |
| peptide | AARQFIGPGTRLTVL | |
| (SignalP) | | |
| Vß | GAVVSQHPSRVICKSGTSVKIECRSLDFQATTM | 2235 |
| without | FWYRQFPKQSLMLMATSNEGSKATYEQGVEKDK | |
| signal | FLINHASLTLSTLTVTSAHPEDSSFYICSGGLE | |
| peptide | EAARQFIGPGTRLTVL | |
| (IMGT) | | |
| Vß | MXLLLLLLGPAGSGLGAVVSQHPSRVICKSGTS | 2236 |
| | VKIECRSLDFQATTMFWYRQFPKQSLMLMATSN | |
| | EGSKATYEQGVEKDKFLINHASLTLSTLTVTSA | |
| | HPEDSSFYICSGGLEEAARQFIGPGTRLTVL | |
| | (X = any amino acid) | 2237 |
| ß chain | MLLLLLLLGPAGSGLGAVVSQHPSRVICKSGTS | 2238 |
| with WT | VKIECRSLDFQATTMFWYRQFPKQSLMLMATSN | |
| signal | AEGSKTYEQGVEKDKFLINHASLTLSTLTVTSA | |
| peptide, | HPEDSSFYICSGGLEEAARQFIGPGTRLTVLED | |
| Cß (sub- | LRNVTPPKVSLFEPSKAEIANKQKATLVCLARG | |
| stituted) | FFPDHVELSWWVNGKEVHSGVCTDPQAYKESNY | |
| | SYCLSSRLRVSATFWHNPRNHFRCQVQFHGLSE | |
| | EDKWPEGSPKPVTQNISAEAWGRADCGITSASY | |
| | QQGVLSATILYEILLGKATLYAVLVSTLVVMAM | |
| | VKRKNS | |
| ß chain | MALLLLLLGPAGSGLGAVVSQHPSRVICKSGTS | 2239 |
| with al- | VKIECRSLDFQATTMFWYRQFPKQSLMLMATSN | |
| ternative | EGSKATYEQGVEKDKFLINHASLTLSTLTVTSA | |
| signal | HPEDSSFYICSGGLEEAARQFIGPGTRLTVLED | |
| peptide, | LRNVTPPKVSLFEPSKAEIANKQKATLVCLARG | |
| Cß (sub- | FFPDHVELSWWVNGKEVHSGVCTDPQAYKESNY | |
| stituted) | SYCLSSRLRVSATFWHNPRNHFRCQVQFHGLSE | |
| | EDKWPEGSPKPVTQNISAEAWGRADCGITSASY | |
| | QQGVLSATILYEILLGKATLYAVLVSTLVVMAM | |
| | VKRKNS | |
| ß chain | MHLLLLLLGPAGSGLGAVVSQHPSRVICKSGTS | 2240 |
| with al- | VKIECRSLDFQATTMFWYRQFPKQSLMLMATSN | |
| ternative | EGSKATYEQGVEKDKFLINHASLTLSTLTVTSA | |
| signal | HPEDSSFYICSGGLEEAARQFIGPGTRLTVLED | |
| peptide, | LRNVTPPKVSLFEPSKAEIANKQKATLVCLARG | |
| Cß (sub- | FFPDHVELSWWVNGKEVHSGVCTDPQAYKESNY | |
| stituted) | SYCLSSRLRVSATFWHNPRNHFRCQVQFHGLSE | |
| | EDKWPEGSPKPVTQNISAEAWGRADCGITSASY | |
| | QQGVLSATILYEILLGKATLYAVLVSTLVVMAM | |
| | VKRKNS | |

In some embodiments, TCR024 interacts with and/or is specific for p53. In some embodiments, the peptide is from a neoantigen of p53. In some embodiments, the neoantigen has the amino acid change R248Q relative to the wild type p53 sequence. In some embodiments, TCR024 interacts with the neoantigen in the context of HLA-A*02:01, as described in International Publication No. WO 2019/067243, incorporated herein by reference in its entirety.

TABLE 6Y
Amino acid sequences of TCR025.

| | | SEQ |
|---|---|---|
| Descrip- | | ID |
| tion | Sequence | NO: |
| CDR1α | TISGNEY | 1241 |
| CDR2α | GLKNN | 1242 |
| CDR3α | IVPNDYKLS | 1243 |
| Vα | KTTQPPSMDCAEGRAANLPCNHSTISGNEYVYW | 1244 |
| without | YRQIHSQGPQYIIHGLKNNETNEMASLIITEDR | |
| signal | KSSTLILPHATLRDTAVYYCIVPNDYKLSFGAG | |
| peptide | TTVTVRA | |
| (SignalP) | | |
| Vα | DAKTTQPPSMDCAEGRAANLPCNHSTISGNEYV | 1245 |
| without | YWYRQIHSQGPQYIIHGLKNNETNEMASLIITE | |
| signal | DRKSSTLILPHATLRDTAVYYCIVPNDYKLSFG | |
| peptide | AGTTVTVRA | |
| (IMGT) | | |
| Vα | MXLVARVTVFLTFGTIIDAKTTQPPSMDCAEGR | 1246 |
| | AANLPCNHSTISGNEYVYWYRQIHSQGPQYIIH | |
| | GLKNNETNEMASLIITEDRKSSTLILPHATLRD | |
| | TAVYYCIVPNDYKLSFGAGTTVTVRA | |
| | (X = any amino acid) | 1247 |
| α chain | MRLVARVTVFLTFGTIIDAKTTQPPSMDCAEGR | 1248 |
| with WT | AANLPCNHSTISGNEYVYWYRQIHSQGPQYIIH | |
| signal | GLKNNETNEMASLIITEDRKSSTLILPHATLRD | |
| peptide, | TAVYYCIVPNDYKLSFGAGTTVTVRANIQNPEP | |
| Cα (sub- | AVYQLKDPRSQDSTLCLFTDFDSQINVPKTMES | |
| stituted) | GTFITDKCVLDMKAMDSKSNGAIAWSNQTSFTC | |
| | YQDIFKETNATPSSDVPCDATLTEKSFETDMNL | |
| | NFQNLLVIVLRILLLKVAGFNLLMTLRLWSS | |
| α chain | MALVARVTVFLTFGTIIDAKTTQPPSMDCAEGR | 1249 |
| with al- | AANLPCNHSTISGNEYVYWYRQIHSQGPQYIIH | |
| ternative | GLKNNETNEMASLIITEDRKSSTLILPHATLRD | |
| signal | TAVYYCIVPNDYKLSFGAGTTVTVRANIQNPEP | |
| peptide, | AVYQLKDPRSQDSTLCLFTDFDSQINVPKTMES | |
| Cα (sub- | GTFITDKCVLDMKAMDSKSNGAIAWSNQTSFTC | |
| stituted) | QDIFKETNATYPSSDVPCDATLTEKSFETDMNL | |
| | SNFQNLLVIVLRILLLKVAGFNLLMTLRLWS | |
| α chain | MHLVARVTVFLTFGTIIDAKTTQPPSMDCAEGR | 1250 |
| with al- | AANLPCNHSTISGNEYVYWYRQIHSQGPQYIIH | |
| ternative | GLKNNETNEMASLIITEDRKSSTLILPHATLRD | |
| signal | TAVYYCIVPNDYKLSFGAGTTVTVRANIQNPEP | |
| peptide, | AVYQLKDPRSQDSTLCLFTDFDSQINVPKTMES | |
| Cα (sub- | GTFITDKCVLDMKAMDSKSNGAIAWSNQTSFTC | |
| stituted) | QDIFKETNATYPSSDVPCDATLTEKSFETDMNL | |
| | NFQNLLVIVLRILLLKVAGFNLLMTLRLWSS | |
| CDR1ß | MDHEN | 2241 |
| CDR2ß | SYDVKM | 2242 |
| CDR3ß | ASSFGTGSIQETQY | 2243 |
| Vß | SRYLVKRTGEKVFLECVQDMDHENMFWYRQDPG | 2244 |
| without | LGLRLIYFSYDVKMKEKGDIPEGYSVSREKKER | |
| signal | FSLILESASTNQTSMYLCASSFGTGSIQETQYF | |
| peptide | GPGTRLLVL | |
| (SignalP) | | |
| Vß | DVKVTQSSRYLVKRTGEKVFLECVQDMDHENMF | 2245 |
| without | WYRQDPGLGLRLIYFSYDVKMKEKGDIPEGYSV | |
| signal | SREKKERFSLILESASTNQTSMYLCASSFGTGS | |
| peptide | IQETQYFGPGTRLLVL | |
| (IMGT) | | |
| Vß | MXIRLLCRVAFCFLAVGLVDVKVTQSSRYLVKR | 2246 |
| | TGEKVFLECVQDMDHENMFWYRQDPGLGLRLIY | |
| | KMFSYDVKEKGDIPEGYSVSREKKERFSLILES | |
| | TASTNQSMYLCASSFGTGSIQETQYFGPGTRLL | |
| | VL | |
| | (X = any amino acid) | 2247 |
| ß chain | MGIRLLCRVAFCFLAVGLVDVKVTQSSRYLVKR | 2248 |
| with WT | TGEKVFLECVQDMDHENMFWYRQDPGLGLRLIY | |
| signal | FSYDVKMKEKGDIPEGYSVSREKKERFSLILES | |
| peptide, | ASTNQTSMYLCASSFGTGSIQETQYFGPGTRLL | |
| Cß (sub- | VLEDLRNVTPPKVSLFEPSKAEIANKQKATLVC | |
| stituted) | ALRGFFPDHVELSWWVNGKEVHSGVCTDPQAYK | |
| | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MAIRLLCRVAFCFLAVGLVDVKVTQSSRYLVKR | 2249 |
| with al- | TGEKVFLECVQDMDHENMFWYRQDPGLGLRLIY | |
| ternative | FSYDVKMKEKGDIPEGYSVSREKKERFSLILES | |
| signal | ASTNQTSMYLCASSFGTGSIQETQYFGPGTRLL | |
| peptide, | VLEDLRNVTPPKVSLFEPSKAEIANKQKATLVC | |
| Cß (sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MHIRLLCRVAFCFLAVGLVDVKVTQSSRYLVKR | 2250 |
| with al- | TGEKVFLECVQDMDHENMFWYRQDPGLGLRLIY | |
| ternative | FSYDVKMKEKGDIPEGYSVSREKKERFSLILES | |
| signal | ASTNQTSMYLCASSFGTGSIQETQYFGPGTRLL | |
| peptide, | VLEDLRNVTPPKVSLFEPSKAEIANKQKATLVC | |
| Cß (sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |

In some embodiments, TCR025 interacts with and/or is specific for p53. In some embodiments, the peptide is from a neoantigen of p53. In some embodiments, the neoantigen has the amino acid change R248Q relative to the wild type p53 sequence. In some embodiments, TCR025 interacts with the neoantigen in the context of HLA-A*02:01, as described in International Publication No. WO 2019/067243, incorporated herein by reference in its entirety.

TABLE 6Z
Amino acid sequences of TCR026

| | | SEQ |
|---|---|---|
| Descrip- | | ID |
| tion | Sequence | NO: |
| CDR1α | ATGYPS | 1251 |
| CDR2α | ATKADDK | 1252 |
| CDR3α | ALNPNAGGTSYGKLT | 1253 |
| Vα | NSVTQMEGPVTLSEEAFLTINCTYTATGYPSLF | 1254 |
| without | WYVQYPGEGLQLLLKATKADDKGSNKGFEATYR | |
| signal | KETTSFHLEKGSVQVSDSAVYFCALNPNAGGTS | |
| peptide | YGKLTFGQGTILTVHP | |
| (SignalP) | | |
| Vα | GNSVTQMEGPVTLSEEAFLTINCTYTATGYPSL | 1255 |
| without | FWYVQYPGEGLQLLLKATKADDKGSNKGFEATY | |
| signal | RKETTSFHLEKGSVQVSDSAVYFCALNPNAGGT | |
| peptide | SYGKLTFGQGTILTVHP | |
| (IMGT) | | |
| Vα | MXYSPGLVSLILLLLGRTRGNSVTQMEGPVTLS | 1256 |
| | EEAFLTINCTYTATGYPSLFWYVQYPGEGLQLL | |
| | LKATKADDKGSNKGFEATYRKETTSFHLEKGSV | |
| | QVSDSAVYFCALNPNAGGTSYGKLTFGQGTILT | |
| | VHP | |
| | (X = any amino acid) | 1257 |
| α chain | MNYSPGLVSLILLLLGRTRGNSVTQMEGPVTLS | 1258 |
| with WT | EEAFLTINCTYTATGYPSLFWYVQYPGEGLQLL | |
| signal | LKATKADDKGSNKGFEATYRKETTSFHLEKGSV | |
| peptide, | QVSDSAVYFCALNPNAGGTSYGKLTFGQGTILT | |
| Cα (sub- | VHPNIQNPEPAVYQLKDPRSQDSTLCLFTDFDS | |
| stituted) | QINVPKTMESGTFITDKCVLDMKAMDSKSNGAI | |
| | AWSNQTSFTCQDIFKETNATYPSSDVPCDATLT | |
| | EKSFETDMNLNFQNLLVIVLRILLLKVAGFNLL | |
| | MTLRLWSS | |
| α chain | MAYSPGLVSLILLLLGRTRGNSVTQMEGPVTLS | 1259 |
| with al- | EEAFLTINCTYTATGYPSLFWYVQYPGEGLQLL | |
| ternative | LKATKADDKGSNKGFEATYRKETTSFHLEKGSV | |
| signal | QVSDSAVYFCALNPNAGGTSYGKLTFGQGTILT | |
| peptide, | VHPNIQNPEPAVYQLKDPRSQDSTLCLFTDFDS | |
| Cα (sub- | QINVPKTMESGTFITDKCVLDMKAMDSKSNGAI | |
| stituted) | AWSNQTSFTCQDIFKETNATYPSSDVPCDATLT | |
| | EKSFETDMNLNFQNLLVIVLRILLLKVAGFNLL | |
| | MTLRLWSS | |
| α chain | MHYSPGLVSLILLLLGRTRGNSVTQMEGPVTLS | 1260 |
| with al- | EEAFLTINCTYTATGYPSLFWYVQYPGEGLQLL | |
| ternative | LKATKADDKGSNKGFEATYRKETTSFHLEKGSV | |
| signal | QVSDSAVYFCALNPNAGGTSYGKLTFGQGTILT | |
| peptide, | VHPNIQNPEPAVYQLKDPRSQDSTLCLFTDFDS | |
| Cα (sub- | QINVPKTMESGTFITDKCVLDMKAMDSKSNGAI | |
| stituted) | AWSNQTSFTCQDIFKETNATYPSSDVPCDATLT | |
| | EKSFETDMNLNFQNLLVIVLRILLLKVAGFNLL | |
| | MTLRLWSS | |
| CDR1ß | SGHTS | 2251 |
| CDR2ß | YDEGEE | 2252 |
| CDR3ß | ASSSPGATSGGANTGELF | 2253 |
| ßV | GVTQSPTHLIKTRGQQATLRCSPISGHTSVYWY | 2254 |
| without | QQALGLGLQFLLWYDEGEERNRGNFPPRFSGRQ | |
| signal | FPNYSSELNVNALELEDSALYLCASSSPGATSG | |
| peptide | GANTGELFFGEGSRLTVL | |
| (SignalP) | | |
| Vß | EAGVTQSPTHLIKTRGQQATLRCSPISGHTSVY | 2255 |
| without | WYQQALGLGLQFLLWYDEGEERNRGNFPPRFSG | |
| signal | RQFPNYSSELNVNALELEDSALYLCASSSPGAT | |
| peptide | SGGANTGELFFGEGSRLTVL | |
| (IMGT) | | |
| Vß | MXPRLLFWALLCLLGTGPVEAGVTQSPTHLIKT | 2256 |
| | RGQQATLRCSPISGHTSVYWYQQALGLGLQFLL | |
| | YDWEGEERNRGNFPPRFSGRQFPNYSSELNVNA | |
| | LELEDSALYLCASSSPGATSGGANTGELFFGEG | |
| | SRLTVL | |
| | (X = any amino acid) | 2257 |
| ß chain | MGPRLLFWALLCLLGTGPVEAGVTQSPTHLIKT | 2258 |
| with WT | RGQQATLRCSPISGHTSVYWYQQALGLGLQFLL | |
| signal | WYDEGEERNRGNFPPRFSGRQFPNYSSELNVNA | |
| peptide, | LELEDSALYLCASSSPGATSGGANTGELFFGEG | |
| Cß (sub- | SRLTVLEDLRNVTPPKVSLFEPSKAEIANKQKA | |
| stituted) | TLVCLARGFFPDHVELSWWVNGKEVHSGVCTDP | |
| | QAYKESNYSYCLSSRLRVSATFWHNPRNHFRCQ | |
| | VQFHGLSEEDKWPEGSPKPVTQNISAEAWGRAD | |
| | CGITSASYQQGVLSATILYEILLGKATLYAVLV | |
| | STLVVMAMVKRKNS | |
| ß chain | MAPRLLFWALLCLLGTGPVEAGVTQSPTHLIKT | 2259 |
| with al- | RGQQATLRCSPISGHTSVYWYQQALGLGLQFLL | |
| ternative | WYDEGEERNRGNFPPRFSGRQFPNYSSELNVNA | |
| signal | LELEDSALYLCASSSPGATSGGANTGELFFGEG | |
| peptide, | SRLTVLEDLRNVTPPKVSLFEPSKAEIANKQKA | |
| Cß (sub- | TLVCLARGFFPDHVELSWWVNGKEVHSGVCTDP | |
| stituted) | QAYKESNYSYCLSSRLRVSATFWHNPRNHFRCQ | |
| | VQFHGLSEEDKWPEGSPKPVTQNISAEAWGRAD | |
| | CGITSASYQQGVLSATILYEILLGKATLYAVLV | |
| | STLVVMAMVKRKNS | |
| ß chain | MHPRLLFWALLCLLGTGPVEAGVTQSPTHLIKT | 2260 |
| with al- | RGQQATLRCSPISGHTSVYWYQQALGLGLQFLL | |
| ternative | WYDEGEERNRGNFPPRFSGRQFPNYSSELNVNA | |
| signal | LELEDSALYLCASSSPGATSGGANTGELFFGEG | |
| peptide, | SRLTVLEDLRNVTPPKVSLFEPSKAEIANKQKA | |
| Cß (sub- | TLVCLARGFFPDHVELSWWVNGKEVHSGVCTDP | |
| stituted) | QAYKESNYSYCLSSRLRVSATFWHNPRNHFRCQ | |
| | VQFHGLSEEDKWPEGSPKPVTQNISAEAWGRAD | |
| | CGITSASYQQGVLSATILYEILLGKATLYAVLV | |
| | STLVVMAMVKRKNS | |

In some embodiments, TCR026 interacts with and/or is specific for p53. In some embodiments, the peptide is from a neoantigen of p53. In some embodiments, the neoantigen has the amino acid change R248Q relative to the wild type p53 sequence. In some embodiments, TCR026 interacts with the neoantigen in the context of HLA-A*02:01, as described in International Publication No. WO 2019/067243, incorporated herein by reference in its entirety.

TABLE 6AA
Amino acid sequences of TCR027.

| | | SEQ |
|---|---|---|
| Descrip- | | ID |
| tion | Sequence | NO: |
| CDR1α | DSSSTY | 1261 |
| CDR2α | IFSNMDM | 1262 |
| CDR3α | AEIPRDSGGGADGLT | 1263 |
| Vα | EDVEQSLFLSVREGDSSVINCTYTDSSSTYLYW | 1264 |
| without | YKQEPGAGLQLLTYIFSNMDMKQDQRLTVLLNK | |
| signal | KDKHLSLRIADTQTGDSAIYFCAEIPRDSGGGA | |
| peptide | DGLTFGKGTHLIIQP | |
| (SignalP) | | |
| Vα | GEDVEQSLFLSVREGDSSVINCTYTDSSSTYLY | 1265 |
| without | WYKQEPGAGLQLLTYIFSNMDMKQDQRLTVLLN | |
| signal | KKDKHLSLRIADTQTGDSAIYFCAEIPRDSGGG | |
| peptide | ADGLTFGKGTHLIIQP | |
| (IMGT) | | |
| Vα | MXTFAGFSFLFLWLQLDCMSRGEDVEQSLFLSV | 1266 |
| | REGDSSVINCTYTDSSSTYLYWYKQEPGAGLQL | |
| | LTYIFSNMDMKQDQRLTVLLNKKDKHLSLRIAD | |
| | TQTGDSAIYFCAEIPRDSGGGADGLTFGKGTHL | |
| | IIQP | |
| | (X = any amino acid) | 1267 |
| α chain | MKTFAGFSFLFLWLQLDCMSRGEDVEQSLFLSV | 1268 |
| with WT | REGDSSVINCTYTDSSSTYLYWYKQEPGAGLQL | |
| signal | LTYIFSNMDMKQDQRLTVLLNKKDKHLSLRIAD | |
| peptide, | TQTGDSAIYFCAEIPRDSGGGADGLTFGKGTHL | |
| Cα (sub- | IIQPNIQNPEPAVYQLKDPRSQDSTLCLFTDFD | |
| stituted) | SQINVPKTMESGTFITDKCVLDMKAMDSKSNGA | |
| | IAWSNQTSFTCQDIFKETNATYPSSDVPCDATL | |
| | TEKSFETDMNLNFQNLLVIVLRILLLKVAGFNL | |
| | LMTLRLWSS | |
| α chain | MATFAGFSFLFLWLQLDCMSRGEDVEQSLFLSV | 1269 |
| with al- | REGDSSVINCTYTDSSSTYLYWYKQEPGAGLQL | |
| ternative | LTYIFSNMDMKQDQRLTVLLNKKDKHLSLRIAD | |
| signal | TQTGDSAIYFCAEIPRDSGGGADGLTFGKGTHL | |
| peptide, | IIQPNIQNPEPAVYQLKDPRSQDSTLCLFTDFD | |
| Cα (sub- | SQINVPKTMESGTFITDKCVLDMKAMDSKSNGA | |
| stituted) | IAWSNQTSFTCQDIFKETNATYPSSDVPCDATL | |
| | TEKSFETDMNLNFQNLLVIVLRILLLKVAGFNL | |
| | LMTLRLWSS | |
| α chain | MHTFAGFSFLFLWLQLDCMSRGEDVEQSLFLSV | 1270 |
| with al- | REGDSSVINCTYTDSSSTYLYWYKQEPGAGLQL | |
| ternative | LTYIFSNMDMKQDQRLTVLLNKKDKHLSLRIAD | |
| signal | TQTGDSAIYFCAEIPRDSGGGADGLTFGKGTHL | |
| peptide, | IIQPNIQNPEPAVYQLKDPRSQDSTLCLFTDFD | |
| Cα (sub- | SQINVPKTMESGTFITDKCVLDMKAMDSKSNGA | |
| stituted) | IAWSNQTSFTCQDIFKETNATYPSSDVPCDATL | |
| | TEKSFETDMNLNFQNLLVIVLRILLLKVAGFNL | |
| | LMTLRLWSS | |
| CDR1ß | DFQATT | 2261 |
| CDR2ß | SNEGSKA | 2262 |
| CDR3ß | SARDLQRSYEQY | 2263 |
| Vß | GSGLGAVVSQHPSWVICKSGTSVKIECRSLDFQ | 2264 |
| without | ATTMFWYRQFPKQSLMLMATSNEGSKATYEQGV | |
| signal | EKDKFLINHASLTLSTLTVTSAHPEDSSFYICS | |
| peptide | ARDLQRSYEQYFGPGTRLTVT | |
| (SignalP) | | |
| Vß | GAVVSQHPSWVICKSGTSVKIECRSLDFQATTM | 2265 |
| without | FWYRQFPKQSLMLMATSNEGSKATYEQGVEKDK | |
| signal | FLINHASLTLSTLTVTSAHPEDSSFYICSARDL | |
| peptide | QRSYEQYFGPGTRLTVT | |
| (IMGT) | | |
| Vß | MXLLLLLLGPGISLLLPGSLAGSGLGAVVSQHP | 2266 |
| | SWVICKSGTSVKIECRSLDFQATTMFWYRQFPK | |
| | QSLMLMATSNEGSKATYEQGVEKDKFLINHASL | |
| | TLSTLTVTSAHPEDSSFYICSARDLQRSYEQYF | |
| | GPGTRLTVT | |
| | (X = any amino acid) | 2267 |
| ß chain | MLLLLLLLGPGISLLLPGSLAGSGLGAVVSQHP | 2268 |
| with WT | SWVICKSGTSVKIECRSLDFQATTMFWYRQFPK | |
| signal | QSLMLMATSNEGSKATYEQGVEKDKFLINHASL | |
| peptide, | TLSTLTVTSAHPEDSSFYICSARDLQRSYEQYF | |
| Cß (sub- | GPGTRLTVTEDLRNVTPPKVSLFEPSKAEIANK | |
| stituted) | QKATLVCLARGFFPDHVELSWWVNGKEVHSGVC | |
| | TDPQAYKESNYSYCLSSRLRVSATFWHNPRNHF | |
| | RCQVQFHGLSEEDKWPEGSPKPVTQNISAEAWG | |
| | RADCGITSASYQQGVLSATILYEILLGKATLYA | |
| | VLVSTLVVMAMVKRKNS | |
| ß chain | MALLLLLLGPGISLLLPGSLAGSGLGAVVSQHP | 2269 |
| with al- | SWVICKSGTSVKIECRSLDFQATTMFWYRQFPK | |
| ternative | QSLMLMATSNEGSKATYEQGVEKDKFLINHASL | |
| signal | TLSTLTVTSAHPEDSSFYICSARDLQRSYEQYF | |
| peptide, | GPGTRLTVTEDLRNVTPPKVSLFEPSKAEIANK | |
| Cß (sub- | QKATLVCLARGFFPDHVELSWWVNGKEVHSGVC | |
| stituted) | TDPQAYKESNYSYCLSSRLRVSATFWHNPRNHF | |
| | RCQVQFHGLSEEDKWPEGSPKPVTQNISAEAWG | |
| | RADCGITSASYQQGVLSATILYEILLGKATLYA | |
| | VLVSTLVVMAMVKRKNS | |
| ß chain | MHLLLLLLGPGISLLLPGSLAGSGLGAVVSQHP | 2270 |
| with al- | SWVICKSGTSVKIECRSLDFQATTMFWYRQFPK | |
| ternative | QSLMLMATSNEGSKATYEQGVEKDKFLINHASL | |
| signal | TLSTLTVTSAHPEDSSFYICSARDLQRSYEQYF | |
| peptide, | GPGTRLTVTEDLRNVTPPKVSLFEPSKAEIANK | |
| Cß (sub- | QKATLVCLARGFFPDHVELSWWVNGKEVHSGVC | |
| stituted) | TDPQAYKESNYSYCLSSRLRVSATFWHNPRNHF | |
| | RCQVQFHGLSEEDKWPEGSPKPVTQNISAEAWG | |
| | RADCGITSASYQQGVLSATILYEILLGKATLYA | |
| | VLVSTLVVMAMVKRKNS | |

In some embodiments, TCR027 interacts with and/or is specific for p53. In some embodiments, the peptide is from a neoantigen of p53. In some embodiments, the neoantigen has the amino acid change R248Q relative to the wild type p53 sequence. In some embodiments, TCR027 interacts with the neoantigen in the context of HLA-A*02:01, as described in International Publication No. WO 2019/067243, incorporated herein by reference in its entirety.

TABLE 6AB
Amino acid sequences of TCR028.

| | | SEQ |
|---|---|---|
| Descrip- | | ID |
| tion | Sequence | NO: |
| CDR1α | DSSSTY | 1271 |
| CDR2α | IFSNMDM | 1272 |
| CDR3α | AEIPRDSGGGADGLT | 1273 |
| Vα | EDVEQSLFLSVREGDSSVINCTYTDSSSTYLYW | 1274 |
| without | YKQEPGAGLQLLTYIFSNMDMKQDQRLTVLLNK | |
| signal | KDKHLSLRIADTQTGDSAIYFCAEIPRDSGGGA | |
| peptide | DGLTFGKGTHLIIQP | |
| (SignalP) | | |
| Vα | GEDVEQSLFLSVREGDSSVINCTYTDSSSTYLY | 1275 |
| without | WYKQEPGAGLQLLTYIFSNMDMKQDQRLTVLLN | |
| signal | KKDKHLSLRIADTQTGDSAIYFCAEIPRDSGGG | |
| peptide | ADGLTFGKGTHLIIQP | |
| (IMGT) | | |
| Vα | MXTFAGFSFLFLWLQLDCMSRGEDVEQSLFLSV | 1276 |
| | REGDSSVINCTYTDSSSTYLYWYKQEPGAGLQL | |
| | LTYIFSNMDMKQDQRLTVLLNKKDKHLSLRIAD | |
| | TQTGDSAIYFCAEIPRDSGGGADGLTFGKGTHL | |
| | IIQP | |
| | (X = any amino acid) | 1277 |
| α chain | MKTFAGFSFLFLWLQLDCMSRGEDVEQSLFLSV | 1278 |
| with WT | REGDSSVINCTYTDSSSTYLYWYKQEPGAGLQL | |
| signal | LTYIFSNMDMKQDQRLTVLLNKKDKHLSLRIAD | |
| peptide, | TQTGDSAIYFCAEIPRDSGGGADGLTFGKGTHL | |
| Cα (sub- | IIQPNIQNPEPAVYQLKDPRSQDSTLCLFTDFD | |
| stituted) | SQINVPKTMESGTFITDKCVLDMKAMDSKSNGA | |
| | IAWSNQTSFTCQDIFKETNATYPSSDVPCDATL | |
| | TEKSFETDMNLNFQNLLVIVLRILLLKVAGFNL | |
| | LMTLRLWSS | |
| α chain | MATFAGFSFLFLWLQLDCMSRGEDVEQSLFLSV | 1279 |
| with al- | REGDSSVINCTYTDSSSTYLYWYKQEPGAGLQL | |
| ternative | LTYIFSNMDMKQDQRLTVLLNKKDKHLSLRIAD | |
| signal | TQTGDSAIYFCAEIPRDSGGGADGLTFGKGTHL | |
| peptide, | IIQPNIQNPEPAVYQLKDPRSQDSTLCLFTDFD | |
| Cα (sub- | SQINVPKTMESGTFITDKCVLDMKAMDSKSNGA | |
| stituted) | IAWSNQTSFTCQDIFKETNATYPSSDVPCDATL | |
| | TEKSFETDMNLNFQNLLVIVLRILLLKVAGFNL | |
| | LMTLRLWSS | |
| α chain | MHTFAGFSFLFLWLQLDCMSRGEDVEQSLFLSV | 1280 |
| with al- | REGDSSVINCTYTDSSSTYLYWYKQEPGAGLQL | |
| ternative | LTYIFSNMDMKQDQRLTVLLNKKDKHLSLRIAD | |
| signal | TQTGDSAIYFCAEIPRDSGGGADGLTFGKGTHL | |
| peptide, | IIQPNIQNPEPAVYQLKDPRSQDSTLCLFTDFD | |
| Cα (sub- | SQINVPKTMESGTFITDKCVLDMKAMDSKSNGA | |
| stituted) | IAWSNQTSFTCQDIFKETNATYPSSDVPCDATL | |
| | TEKSFETDMNLNFQNLLVIVLRILLLKVAGFNL | |
| | LMTLRLWSS | |
| CDR1ß | DFQATT | 2271 |
| CDR2ß | SNEGSKA | 2272 |
| CDR3ß | SARDLQRSYEQY | 2273 |
| Vß | AVVSQHPSRVICKSGTSVKIECRSLDFQATTMF | 2274 |
| without | WYRQFPKQSLMLMATSNEGSKATYEQGVEKDKF | |
| signal | LINHASLTLSTLTVTSAHPEDSSFYICSARDLQ | |
| peptide | RSYEQYFGPGTRLTVT | |
| (SignalP) | | |
| Vß | GAVVSQHPSRVICKSGTSVKIECRSLDFQATTM | 2275 |
| without | FWYRQFPKQSLMLMATSNEGSKATYEQGVEKDK | |
| signal | FLINHASLTLSTLTVTSAHPEDSSFYICSARDL | |
| peptide | QRSYEQYFGPGTRLTVT | |
| (IMGT) | | |
| Vß | MXLLLLLLGPAGSGLGAVVSQHPSRVICKSGTS | 2276 |
| | VKIECRSLDFQATTMFWYRQFPKQSLMLMATSN | |
| | EGSKATYEQGVEKDKFLINHASLTLSTLTVTSA | |
| | SHPEDSFYICSARDLQRSYEQYFGPGTRLTVT | |
| | (X = any amino acid) | 2277 |
| ß chain | MLLLLLLLGPAGSGLGAVVSQHPSRVICKSGTS | 2278 |
| with WT | VKIECRSLDFQATTMFWYRQFPKQSLMLMATSN | |
| signal | EGSKATYEQGVEKDKFLINHASLTLSTLTVTSA | |
| peptide, | HPEDSSFYICSARDLQRSYEQYFGPGTRLTVTE | |
| Cß (sub- | DLRNVTPPKVSLFEPSKAEIANKQKATLVCLAR | |
| stituted) | GFFPDHVELSWWVNGKEVHSGVCTDPQAYKESN | |
| | YSYCLSSRLRVSATFWHNPRNHFRCQVQFHGLS | |
| | EEDKWPEGSPKPVTQNISAEAWGRADCGITSAS | |
| | YQQGVLSATILYEILLGKATLYAVLVSTLVVMA | |
| | MVKRKNS | |
| ß chain | MALLLLLLGPAGSGLGAVVSQHPSRVICKSGTS | 2279 |
| with al- | VKIECRSLDFQATTMFWYRQFPKQSLMLMATSN | |
| ternative | EGSKATYEQGVEKDKFLINHASLTLSTLTVTSA | |
| signal | HPEDSSFYICSARDLQRSYEQYFGPGTRLTVTE | |
| peptide, | DLRNVTPPKVSLFEPSKAEIANKQKATLVCLAR | |
| Cß (sub- | GFFPDHVELSWWVNGKEVHSGVCTDPQAYKESN | |
| stituted) | YSYCLSSRLRVSATFWHNPRNHFRCQVQFHGLS | |
| | EEDKWPEGSPKPVTQNISAEAWGRADCGITSAS | |
| | YQQGVLSATILYEILLGKATLYAVLVSTLVVMA | |
| | MVKRKNS | |
| ß chain | MHLLLLLLGPAGSGLGAVVSQHPSRVICKSGTS | 2280 |
| with al- | VKIECRSLDFQATTMFWYRQFPKQSLMLMATSN | |
| ternative | EGSKATYEQGVEKDKFLINHASLTLSTLTVTSA | |
| signal | HPEDSSFYICSARDLQRSYEQYFGPGTRLTVTE | |
| peptide, | DLRNVTPPKVSLFEPSKAEIANKQKATLVCLAR | |
| Cß (sub- | GFFPDHVELSWWVNGKEVHSGVCTDPQAYKESN | |
| stituted) | YSYCLSSRLRVSATFWHNPRNHFRCQVQFHGLS | |
| | EEDKWPEGSPKPVTQNISAEAWGRADCGITSAS | |
| | YQQGVLSATILYEILLGKATLYAVLVSTLVVMA | |
| | MVKRKNS | |

In some embodiments, TCR028 interacts with and/or is specific for p53. In some embodiments, the peptide is from a neoantigen of p53. In some embodiments, the neoantigen has the amino acid change R248Q relative to the wild type p53 sequence. In some embodiments, TCR028 interacts with the neoantigen in the context of HLA-A*02:01, as described in International Publication No. WO 2019/067243, incorporated herein by reference in its entirety.

TABLE 6AC
Amino acid sequences of TCR029.

| | | SEQ |
|---|---|---|
| Descrip- | | ID |
| tion | Sequence | NO: |
| CDR1α | NSASDY | 1281 |
| CDR2α | IRSNMDK | 1282 |
| CDR3α | AEPVGGLNSGYALN | 1283 |
| Vα | ESVGLHLPTLSVQEGDNSIINCAYSNSASDYFI | 1284 |
| without | WYKQESGKGPQFIIDIRSNMDKRQGQRVTVLLN | |
| signal | KTVKHLSLQIAATQPGDSAVYFCAEPVGGLNSG | |
| peptide | YALNFGKGTSLLVTP | |
| (SignalP) | | |
| Vα | GESVGLHLPTLSVQEGDNSIINCAYSNSASDYF | 1285 |
| without | IWYKQESGKGPQFIIDIRSNMDKRQGQRVTVLL | |
| signal | NKTVKHLSLQIAATQPGDSAVYFCAEPVGGLNS | |
| peptide | GYALNFGKGTSLLVTP | |
| (IMGT) | | |
| Vα | MXGIRALFMYLWLQLDWVSRGESVGLHLPTLSV | 1286 |
| | QEGDNSIINCAYSNSASDYFIWYKQESGKGPQF | |
| | IIDIRSNMDKRQGQRVTVLLNKTVKHLSLQIAA | |
| | TQPGDSAVYFCAEPVGGLNSGYALNFGKGTSLL | |
| | VTP | |
| | (X = any amino acid) | 1287 |
| α chain | MAGIRALFMYLWLQLDWVSRGESVGLHLPTLSV | 1288 |
| with WT | QEGDNSIINCAYSNSASDYFIWYKQESGKGPQF | |
| signal | IIDIRSNMDKRQGQRVTVLLNKTVKHLSLQIAA | |
| peptide, | TQPGD | |
| Cα (sub- | SAVYFCAEPVGGLNSGYALNFGKGTSLLVTPNI | 1289 |
| stituted) | QNPEPAVYQLKDPRSQDSTLCLFTDFDSQINVP | |
| | KTMESGTFITDKCVLDMKAMDSKSNGAIAWSNQ | |
| | TSFTCQDIFKETNATYPSSDVPCDATLTEKSFE | |
| | TDMNLNFQNLLVIVLRILLLKVAGFNLLMTLRL | |
| | WSS | |
| α chain | MHGIRALFMYLWLQLDWVSRGESVGLHLPTLSV | 1290 |
| with al- | QEGDNSIINCAYSNSASDYFIWYKQESGKGPQF | |
| ternative | IIDIRSNMDKRQGQRVTVLLNKTVKHLSLQIAA | |
| signal | TQPGDSAVYFCAEPVGGLNSGYALNFGKGTSLL | |
| peptide, | VTPNIQNPEPAVYQLKDPRSQDSTLCLFTDFDS | |
| Cα (sub- | QINVPKTMESGTFITDKCVLDMKAMDSKSNGAI | |
| stituted) | AWSNQTSFTCQDIFKETNATYPSSDVPCDATLT | |
| | EKSFETDMNLNFQNLLVIVLRILLLKVAGFNLL | |
| | MTLRLWSS | |
| CDR1ß | SGHKS | 2281 |
| CDR2ß | QYYEKEE | 2282 |
| CDR3ß | ASSGGRTSGAYEQF | 2283 |
| Vß | GVTQSPTHLIKTRGQQVTLRCSPISGHKSVSWY | 2284 |
| without | QQVLGQGPQFIFQYYEKEERGRGNFPDRFSARQ | |
| signal | FPNYSSELNVNALLLGDSALYLCASSGGRTSGA | |
| peptide | YEQFFGPGTRLTVL | |
| (SignalP) | | |
| Vß | DAGVTQSPTHLIKTRGQQVTLRCSPISGHKSVS | 2285 |
| without | WYQQVLGQGPQFIFQYYEKEERGRGNFPDRFSA | |
| signal | RQFPNYSSELNVNALLLGDSALYLCASSGGRTS | |
| peptide | GAYEQFFGPGTRLTVL | |
| (IMGT) | | |
| Vß | MXPGLLCWVLLCLLGAGPVDAGVTQSPTHLIKT | 2286 |
| | RGQQVTLRCSPISGHKSVSWYQQVLGQGPQFIF | |
| | QYYEKEERGRGNFPDRFSARQFPNYSSELNVNA | |
| | LLLGDSALYLCASSGGRTSGAYEQFFGPGTRLT | |
| | VL | |
| | (X = any amino acid) | 2287 |
| ß chain | MGPGLLCWVLLCLLGAGPVDAGVTQSPTHLIKT | 2288 |
| with WT | RGQQVTLRCSPISGHKSVSWYQQVLGQGPQFIF | |
| signal | QYYEKEERGRGNFPDRESARQFPNYSSELNVNA | |
| peptide, | LLLGDSALYLCASSGGRTSGAYEQFFGPGTRLT | |
| Cß (sub- | VLEDLRNVTPPKVSLFEPSKAEIANKQKATLVC | |
| stituted) | LARGFFPDHVELSWWVNGKEVHSGVCTDPQAYK | |
| | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MAPGLLCWVLLCLLGAGPVDAGVTQSPTHLIKT | 2289 |
| with al- | RGQQVTLRCSPISGHKSVSWYQQVLGQGPQFIF | |
| ternative | QYYEKEERGRGNFPDRFSARQFPNYSSELNVNA | |
| signal | LLLGDSALYLCASSGGRTSGAYEQFFGPGTRLT | |
| peptide, | VLEDLRNVTPPKVSLFEPSKAEIANKQKATLVC | |
| Cß (sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MHPGLLCWVLLCLLGAGPVDAGVTQSPTHLIKT | 2290 |
| with al- | RGQQVTLRCSPISGHKSVSWYQQVLGQGPQFIF | |
| ternative | QYYEKEERGRGNFPDRFSARQFPNYSSELNVNA | |
| signal | LLLGDSALYLCASSGGRTSGAYEQFFGPGTRLT | |
| peptide, | VLEDLRNVTPPKVSLFEPSKAEIANKQKATLVC | |
| Cß (sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |

In some embodiments, TCR029 interacts with and/or is specific for p53. In some embodiments, the peptide is from a neoantigen of p53. In some embodiments, the neoantigen has the amino acid change R248Q relative to the wild type p53 sequence. In some embodiments, TCR029 interacts with the neoantigen in the context of HLA-A*02:01, as described in

TABLE 6AD
Amino acid sequences of TCR030.

| | | SEQ |
|---|---|---|
| Descrip- | | ID |
| tion | Sequence | NO: |
| CDR1α | VSGLRG | 1291 |
| CDR2α | LYSAGEE | 1292 |
| CDR3α | AVTAHRGSTLGRLY | 1293 |
| Vα | EDQVTQSPEALRLQEGESSSLNCSYTVSGLRGL | 1294 |
| without | FWYRQDPGKGPEFLFTLYSAGEEKEKERLKATL | |
| signal | TKKESFLHITAPKPEDSATYLCAVTAHRGSTLG | |
| peptide | RLYFGRGTQLTVWP | |
| (SignalP) | | |
| Vα | EDQVTQSPEALRLQEGESSSLNCSYTVSGLRGL | 1295 |
| without | FWYRQDPGKGPEFLFTLYSAGEEKEKERLKATL | |
| signal | TKKESFLHITAPKPEDSATYLCAVTAHRGSTLG | |
| peptide | RLYFGRGTQLTVWP | |
| (IMGT) | | |
| Vα | MXKMLECAFIVLWLQLGWLSGEDQVTQSPEALR | 1296 |
| | LQEGESSSLNCSYTVSGLRGLFWYRQDPGKGPE | |
| | FLFTLYSAGEEKEKERLKATLTKKESFLHITAP | |
| | KPEDSATYLCAVTAHRGSTLGRLYFGRGTQLTV | |
| | WP | |
| | (X = any amino acid) | 1297 |
| α chain | MEKMLECAFIVLWLQLGWLSGEDQVTQSPEALR | 1298 |
| with WT | LQEGESSSLNCSYTVSGLRGLFWYRQDPGKGPE | |
| signal | FLFTLYSAGEEKEKERLKATLTKKESFLHITAP | |
| peptide, | KPEDSATYLCAVTAHRGSTLGRLYFGRGTQLTV | |
| Cα (sub- | WPNIQNPEPAVYQLKDPRSQDSTLCLFTDFDSQ | |
| stituted) | INVPKTMESGTFITDKCVLDMKAMDSKSNGAIA | |
| | WSNQTSFTCQDIFKETNATYPSSDVPCDATLTE | |
| | KSFETDMNLNFQNLLVIVLRILLLKVAGFNLLM | |
| | TLRLWSS | |
| α chain | MAKMLECAFIVLWLQLGWLSGEDQVTQSPEALR | 1299 |
| with al- | LQEGESSSLNCSYTVSGLRGLFWYRQDPGKGPE | |
| ternative | FLFTLYSAGEEKEKERLKATLTKKESFLHITAP | |
| signal | KPEDSATYLCAVTAHRGSTLGRLYFGRGTQLTV | |
| peptide, | WPNIQNPEPAVYQLKDPRSQDSTLCLFTDFDSQ | |
| Cα (sub- | INVPKTMESGTFITDKCVLDMKAMDSKSNGAIA | |
| stituted) | WSNQTSFTCQDIFKETNATYPSSDVPCDATLTE | |
| | KSFETDMNLNFQNLLVIVLRILLLKVAGFNLLM | |
| | TLRLWSS | |
| α chain | MHKMLECAFIVLWLQLGWLSGEDQVTQSPEALR | 1300 |
| with al- | LQEGESSSLNCSYTVSGLRGLFWYRQDPGKGPE | |
| ternative | FLFTLYSAGEEKEKERLKATLTKKESFLHITAP | |
| signal | KPEDSATYLCAVTAHRGSTLGRLYFGRGTQLTV | |
| peptide, | WPNIQNPEPAVYQLKDPRSQDSTLCLFTDFDSQ | |
| Cα (sub- | INVPKTMESGTFITDKCVLDMKAMDSKSNGAIA | |
| stituted) | WSNQTSFTCQDIFKETNATYPSSDVPCDATLTE | |
| | KSFETDMNLNFQNLLVIVLRILLLKVAGFNLLM | |
| | TLRLWSS | |
| CDR1ß | SGHDT | 2291 |
| CDR2ß | YYEEEE | 2292 |
| CDR3ß | ASSRRGGAYNEQF | 2293 |
| Vß | GVTQSPTHLIKTRGQQVTLRCSPKSGHDTVSWY | 2294 |
| without | QQALGQGPQFIFQYYEEEERQRGNFPDRFSGHQ | |
| signal | FPNYSSELNVNALLLGDSALYLCASSRRGGAYN | |
| peptide | EQFFGPGTRLTVL | |
| (SignalP) | | |
| Vß | DAGVTQSPTHLIKTRGQQVTLRCSPKSGHDTVS | 2295 |
| without | WYQQALGQGPQFIFQYYEEEERQRGNFPDRFSG | |
| signal | HQFPNYSSELNVNALLLGDSALYLCASSRRGGA | |
| peptide | YNEQFFGPGTRLTVL | |
| (IMGT) | | |
| Vß | MXPGLLCWALLCLLGAGLVDAGVTQSPTHLIKT | 2296 |
| | RGQQVTLRCSPKSGHDTVSWYQQALGQGPQFIF | |
| | QYYEEEERQRGNFPDRFSGHQFPNYSSELNVNA | |
| | LLLGDSALYLCASSRRGGAYNEQFFGPGTRLTV | |
| | L | |
| | (X = any amino acid) | 2297 |
| ß chain | MGPGLLCWALLCLLGAGLVDAGVTQSPTHLIKT | 2298 |
| with WT | RGQQVTLRCSPKSGHDTVSWYQQALGQGPQFIF | |
| signal | QYYEEEERQRGNFPDRFSGHQFPNYSSELNVNA | |
| peptide, | LLLGDSALYLCASSRRGGAYNEQFFGPGTRLTV | |
| Cß (sub- | LEDLRNVTPPKVSLFEPSKAEIANKQKATLVCL | |
| stituted) | ARGFFPDHVELSWWVNGKEVHSGVCTDPQAYKE | |
| | SNYSYCLSSRLRVSATFWHNPRNHFRCQVQFHG | |
| | LSEEDKWPEGSPKPVTQNISAEAWGRADCGITS | |
| | ASYQQGVLSATILYEILLGKATLYAVLVSTLVV | |
| | MAMVKRKNS | |
| ß chain | MAPGLLCWALLCLLGAGLVDAGVTQSPTHLIKT | 2299 |
| with al- | RGQQVTLRCSPKSGHDTVSWYQQALGQGPQFIF | |
| ternative | QYYEEEERQRGNFPDRESGHQFPNYSSELNVNA | |
| signal | LLLGDSALYLCASSRRGGAYNEQFFGPGTRLTV | |
| peptide, | LEDLRNVTPPKVSLFEPSKAEIANKQKATLVCL | |
| Cß(sub- | ARGFFPDHVELSWWVNGKEVHSGVCTDPQAYKE | |
| stituted) | SNYSYCLSSRLRVSATFWHNPRNHFRCQVQFHG | |
| | LSEEDKWPEGSPKPVTQNISAEAWGRADCGITS | |
| | ASYQQGVLSATILYEILLGKATLYAVLVSTLVV | |
| | MAMVKRKNS | |
| ß chain | MHPGLLCWALLCLLGAGLVDAGVTQSPTHLIKT | 2300 |
| with al- | RGQQVTLRCSPKSGHDTVSWYQQALGQGPQFIF | |
| ternative | QYYEEEERQRGNFPDRESGHQFPNYSSELNVNA | |
| signal | LLLGDSALYLCASSRRGGAYNEQFFGPGTRLTV | |
| peptide, | LEDLRNVTPPKVSLFEPSKAEIANKQKATLVCL | |
| Cß (sub- | ARGFFPDHVELSWWVNGKEVHSGVCTDPQAYKE | |
| stituted) | SNYSYCLSSRLRVSATFWHNPRNHFRCQVQFHG | |
| | LSEEDKWPEGSPKPVTQNISAEAWGRADCGITS | |
| | ASYQQGVLSATILYEILLGKATLYAVLVSTLVV | |
| | MAMVKRKNS | |

In some embodiments, TCR030 interacts with and/or is specific for p53. In some embodiments, the peptide is from a neoantigen of p53. In some embodiments, the neoantigen has the amino acid change R248Q relative to the wild type p53 sequence. In some embodiments, TCR030 interacts with the neoantigen in the context of HLA-A*02:01, as described in International Publication No. WO 2019/067243, incorporated herein by reference in its entirety.

TABLE 6AE
Amino acid sequences of TCR031.

| | | SEQ |
|---|---|---|
| Descrip- | | ID |
| tion | Sequence | NO: |
| CDR1α | SSNFYA | 1301 |
| CDR2α | MTLNGDE | 1302 |
| CDR3α | ASVGGGADGLT | 1303 |
| Vα | ILNVEQSPQSLHVQEGDSTNFTCSFPSSNFYAL | 1304 |
| without | HWYRWETAKSPEALFVMTLNGDEKKKGRISATL | |
| signal | NTKEGYSYLYIKGSQPEDSATYLCASVGGGADG | |
| peptide | LTFGKGTHLIIQP | |
| (SignalP) | | |
| Vα | ILNVEQSPQSLHVQEGDSTNFTCSFPSSNFYAL | 1305 |
| without | HWYRWETAKSPEALFVMTLNGDEKKKGRISATL | |
| signal | NTKEGYSYLYIKGSQPEDSATYLCASVGGGADG | |
| peptide | LTFGKGTHLIIQP | |
| (IMGT) | | |
| Vα | MXKNPLAAPLLILWFHLDCVSSILNVEQSPQSL | 1306 |
| | HVQEGDSTNFTCSFPSSNFYALHWYRWETAKSP | |
| | EALFVMTLNGDEKKKGRISATLNTKEGYSYLYI | |
| | KGSQPEDSATYLCASVGGGADGLTFGKGTHLII | |
| | QP | |
| | (X = any amino acid) | 1307 |
| α chain | MEKNPLAAPLLILWFHLDCVSSILNVEQSPQSL | 1308 |
| with WT | HVQEGDSTNFTCSFPSSNFYALHWYRWETAKSP | |
| signal | EALFVMTLNGDEKKKGRISATLNTKEGYSYLYI | |
| peptide, | KGSQPEDSATYLCASVGGGADGLTFGKGTHLII | |
| Cα (sub- | QPNIQNPEPAVYQLKDPRSQDSTLCLFTDFDSQ | |
| stituted) | INVPKTMESGTFITDKCVLDMKAMDSKSNGAIA | |
| | WSNQTSFTCQDIFKETNATYPSSDVPCDATLTE | |
| | KSFETDMNLNFQNLLVIVLRILLLKVAGFNLLM | |
| | TLRLWSS | |
| α chain | MAKNPLAAPLLILWFHLDCVSSILNVEQSPQSL | 1309 |
| with al- | HVQEGDSTNFTCSFPSSNFYALHWYRWETAKSP | |
| ternative | EALFVMTLNGDEKKKGRISATLNTKEGYSYLYI | |
| signal | KGSQPEDSATYLCASVGGGADGLTFGKGTHLII | |
| peptide, | QPNIQNPEPAVYQLKDPRSQDSTLCLFTDFDSQ | |
| Cα (sub- | INVPKTMESGTFITDKCVLDMKAMDSKSNGAIA | |
| stituted) | WSNQTSFTCQDIFKETNATYPSSDVPCDATLTE | |
| | KSFETDMNLNFQNLLVIVLRILLLKVAGFNLLM | |
| | TLRLWSS | |
| α chain | MHKNPLAAPLLILWFHLDCVSSILNVEQSPQSL | 1310 |
| with al- | HVQEGDSTNFTCSFPSSNFYALHWYRWETAKSP | |
| ternative | EALFVMTLNGDEKKKGRISATLNTKEGYSYLYI | |
| signal | KGSQPEDSATYLCASVGGGADGLTFGKGTHLII | |
| peptide, | QPNIQNPEPAVYQLKDPRSQDSTLCLFTDFDSQ | |
| Cα (sub- | INVPKTMESGTFITDKCVLDMKAMDSKSNGAIA | |
| stituted) | WSNQTSFTCQDIFKETNATYPSSDVPCDATLTE | |
| | KSFETDMNLNFQNLLVIVLRILLLKVAGFNLLM | |
| | TLRLWSS | |
| CDR1ß | SGHTA | 2301 |
| CDR2ß | FQGNSA | 2302 |
| CDR3ß | ASTWDRGSYNEQF | 2303 |
| Vß | GVSQSPSNKVTEKGKDVELRCDPISGHTALYWY | 2304 |
| without | RQSLGQGLEFLIYFQGNSAPDKSGLPSDRFSAE | |
| signal | RTGGSVSTLTIQRTQQEDSAVYLCASTWDRGSY | |
| peptide | NEQFFGPGTRLTVL | |
| (SignalP) | | |
| Vß | GAGVSQSPSNKVTEKGKDVELRCDPISGHTALY | 2305 |
| without | WYRQSLGQGLEFLIYFQGNSAPDKSGLPSDRFS | |
| signal | AERTGGSVSTLTIQRTQQEDSAVYLCASTWDRG | |
| peptide | SYNEQFFGPGTRLTVL | |
| (IMGT) | | |
| Vß | MXTRLLFWVAFCLLGADHTGAGVSQSPSNKVTE | 2306 |
| | KGKDVELRCDPISGHTALYWYRQSLGQGLEFLI | |
| | YFQGNSAPDKSGLPSDRFSAERTGGSVSTLTIQ | |
| | RTQQEDSAVYLCASTWDRGSYNEQFFGPGTRLT | |
| | VL | |
| | (X = any amino acid) | 2307 |
| ß chain | MGTRLLFWVAFCLLGADHTGAGVSQSPSNKVTE | 2308 |
| with WT | KGKDVELRCDPISGHTALYWYRQSLGQGLEFLI | |
| signal | YFQGNSAPDKSGLPSDRFSAERTGGSVSTLTIQ | |
| peptide, | RTQQEDSAVYLCASTWDRGSYNEQFFGPGTRLT | |
| Cß (sub- | VLEDLRNVTPPKVSLFEPSKAEIANKQKATLVC | |
| stituted) | LARGFFPDHVELSWWVNGKEVHSGVCTDPQAYK | |
| | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MATRLLFWVAFCLLGADHTGAGVSQSPSNKVTE | 2309 |
| with al- | KGKDVELRCDPISGHTALYWYRQSLGQGLEFLI | |
| ternative | YFQGNSAPDKSGLPSDRFSAERTGGSVSTLTIQ | |
| signal | RTQQEDSAVYLCASTWDRGSYNEQFFGPGTRLT | |
| peptide, | VLEDLRNVTPPKVSLFEPSKAEIANKQKATLVC | |
| (Cß sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MHTRLLFWVAFCLLGADHTGAGVSQSPSNKVTE | 2310 |
| with al- | KGKDVELRCDPISGHTALYWYRQSLGQGLEFLI | |
| ternative | YFQGNSAPDKSGLPSDRFSAERTGGSVSTLTIQ | |
| signal | RTQQEDSAVYLCASTWDRGSYNEQFFGPGTRLT | |
| peptide, | VLEDLRNVTPPKVSLFEPSKAEIANKQKATLVC | |
| Cß (sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |

In some embodiments, TCR031 interacts with and/or is specific for p53. In some embodiments, the peptide is from a neoantigen of p53. In some embodiments, the neoantigen has the amino acid change R248Q relative to the wild type p53 sequence. In some embodiments, TCR031 interacts with the neoantigen in the context of HLA-A*02:01, as described in

TABLE 6AF
Amino acid sequences of TCR032.

| | | SEQ |
|---|---|---|
| Descrip- | | ID |
| tion | Sequence | NO: |
| CDR1α | NSMFDY | 1311 |
| CDR2α | ISSIKDK | 1312 |
| CDR3α | AANTGNQFY | 1313 |
| Vα | QQKNDDQQVKQNSPSLSVQEGRISILNCDYTNS | 1314 |
| without | MFDYFLWYKKYPAEGPTFLISISSIKDKNEDGR | |
| signal | FTVFLNKSAKHLSLHIVPSQPGDSAVYFCAANT | |
| peptide | GNQFYFGTGTSLTVIP | |
| (SignalP) | | |
| Vα | DQQVKQNSPSLSVQEGRISILNCDYTNSMFDYF | 1315 |
| without | LWYKKYPAEGPTFLISISSIKDKNEDGRFTVFL | |
| signal | NKSAKHLSLHIVPSQPGDSAVYFCAANTGNQFY | |
| peptide) | FGTGTSLTVIP | |
| (IMGT | | |
| Vα | MXMLLGASVLILWLQPDWVNSQQKNDDQQVKQN | 1316 |
| | SPSLSVQEGRISILNCDYTNSMFDYFLWYKKYP | |
| | AEGPTFLISISSIKDKNEDGRFTVFLNKSAKHL | |
| | SLHIVPSQPGDSAVYFCAANTGNQFYFGTGTSL | |
| | TVIP | |
| | (X = any amino acid) | 1317 |
| α chain | MAMLLGASVLILWLQPDWVNSQQKNDDQQVKQN | 1318 |
| with WT | SPSLSVQEGRISILNCDYTNSMFDYFLWYKKYP | |
| signal | AEGPTFLISISSIKDKNEDGRFTVFLNKSAKHL | |
| peptide, | SLHIVPSQPGDSAVYFCAANTGNQFYFGTGTSL | |
| Cα (sub- | TVIPNIQNPEPAVYQLKDPRSQDS | |
| stituted) | TLCLFTDFDSQINVPKTMESGTFITDKCVLDMK | 1319 |
| | AMDSKSNGAIAWSNQTSFTCQDIFKETNATYPS | |
| | SDVPCDATLTEKSFETDMNLNFQNLLVIVLRIL | |
| | LLKVAGFNLLMTLRLWSS | |
| α chain | MHMLLGASVLILWLQPDWVNSQQKNDDQQVKQN | 1320 |
| with al- | SPSLSVQEGRISILNCDYTNSMFDYFLWYKKYP | |
| ternative | AEGPTFLISISSIKDKNEDGRFTVFLNKSAKHL | |
| signal | SLHIVPSQPGDSAVYFCAANTGNQFYFGTGTSL | |
| peptide, | TVIPNIQNPEPAVYQLKDPRSQDSTLCLFTDFD | |
| Cα (sub- | SQINVPKTMESGTFITDKCVLDMKAMDSKSNGA | |
| stituted) | IAWSNQTSFTCQDIFKETNATYPSSDVPCDATL | |
| | TEKSFETDMNLNFQNLLVIVLRILLLKVAGFNL | |
| | LMTLRLWSS | |
| CDR1ß | SGHAT | 2311 |
| CDR2ß | FQNNGV | 2312 |
| CDR3ß | ASSHLAGEFYNEQF | 2313 |
| Vß | GVAQSPRYKIIEKRQSVAFWCNPISGHATLYWY | 2314 |
| without | QQILGQGPKLLIQFQNNGVVDDSQLPKDRFSAE | |
| signal | RLKGVDSTLKIQPAKLEDSAVYLCASSHLAGEF | |
| peptide | YNEQFFGPGTRLTVL | |
| (SignalP) | | |
| Vß | EAGVAQSPRYKIIEKRQSVAFWCNPISGHATLY | 2315 |
| without | WYQQILGQGPKLLIQFQNNGVVDDSQLPKDRFS | |
| signal | AERLKGVDSTLKIQPAKLEDSAVYLCASSHLAG | |
| peptide | EFYNEQFFGPGTRLTVL | |
| (IMGT) | | |
| Vß | MXTRLLCWAALCLLGAELTEAGVAQSPRYKIIE | 2316 |
| | KRQSVAFWCNPISGHATLYWYQQILGQGPKLLI | |
| | QFQNNGVVDDSQLPKDRFSAERLKGVDSTLKIQ | |
| | PAKLEDSAVYLCASSHLAGEFYNEQFFGPGTRL | |
| | TVL | |
| | (X = any amino acid) | 2317 |
| ß chain | MGTRLLCWAALCLLGAELTEAGVAQSPRYKIIE | 2318 |
| with WT | KRQSVAFWCNPISGHATLYWYQQILGQGPKLLI | |
| signal | QFQNNGVVDDSQLPKDRFSAERLKGVDSTLKIQ | |
| peptide, | PAKLEDSAVYLCASSHLAGEFYNEQFFGPGTRL | |
| Cß (sub- | TVLEDLRNVTPPKVSLFEPSKAEIANKQKATLV | |
| stituted) | LCARGFFPDHVELSWWVNGKEVHSGVCTDPQAY | |
| | KESNYSYCLSSRLRVSATFWHNPRNHFRCQVQF | |
| | HGLSEEDKWPEGSPKPVTQNISAEAWGRADCGI | |
| | TSASYQQGVLSATILYEILLGKATLYAVLVSTL | |
| | VVMAMVKRKNS | |
| ß chain | MATRLLCWAALCLLGAELTEAGVAQSPRYKIIE | 2319 |
| with al- | KRQSVAFWCNPISGHATLYWYQQILGQGPKLLI | |
| ternative | QFQNNGVVDDSQLPKDRFSAERLKGVDSTLKIQ | |
| signal | PAKLEDSAVYLCASSHLAGEFYNEQFFGPGTRL | |
| peptide, | TVLEDLRNVTPPKVSLFEPSKAEIANKQKATLV | |
| Cß (sub- | CLARGFFPDHVELSWWVNGKEVHSGVCTDPQAY | |
| stituted) | KESNYSYCLSSRLRVSATFWHNPRNHFRCQVQF | |
| | HGLSEEDKWPEGSPKPVTQNISAEAWGRADCGI | |
| | TSASYQQGVLSATILYEILLGKATLYAVLVSTL | |
| | VVMAMVKRKNS | |
| ß chain | MHTRLLCWAALCLLGAELTEAGVAQSPRYKIIE | 2320 |
| with al- | KRQSVAFWCNPISGHATLYWYQQILGQGPKLLI | |
| ternative | QFQNNGVVDDSQLPKDRFSAERLKGVDSTLKIQ | |
| signal | PAKLEDSAVYLCASSHLAGEFYNEQFFGPGTRL | |
| peptide, | TVLEDLRNVTPPKVSLFEPSKAEIANKQKATLV | |
| Cß (sub- | CLARGFFPDHVELSWWVNGKEVHSGVCTDPQAY | |
| stituted) | KESNYSYCLSSRLRVSATFWHNPRNHFRCQVQF | |
| | HGLSEEDKWPEGSPKPVTQNISAEAWGRADCGI | |
| | TSASYQQGVLSATILYEILLGKATLYAVLVSTL | |
| | VVMAMVKRKNS | |

In some embodiments, TCR032 interacts with and/or is specific for p53. In some embodiments, the peptide is from a neoantigen of p53. In some embodiments, the neoantigen has the amino acid change R248Q relative to the wild type p53 sequence. In some embodiments, TCR032 interacts with the neoantigen in the context of HLA-A*02:01, as described in International Publication No. WO 2019/067243, incorporated herein by reference in its entirety.

TABLE 6AG
Amino acid sequences of TCR033.

| | | SEQ |
|---|---|---|
| Descrip- | | ID |
| tion | Sequence | NO: |
| CDR1α | TSGFYG | 1321 |
| CDR2α | NALDGL | 1322 |
| CDR3α | AFAYGQNFV | 1323 |
| Vα | QSLEQPSEVTAVEGAIVQINCTYQTSGFYGLSW | 1324 |
| without | YQQHDGGAPTFLSYNALDGLEETGRESSFLSRS | |
| signal | DSYGYLLLQELQMKDSASYFCAFAYGQNFVFGP | |
| peptide | GTRLSVLP | |
| (SignalP) | | |
| Vα | GQSLEQPSEVTAVEGAIVQINCTYQTSGFYGLS | 1325 |
| without | WYQQHDGGAPTFLSYNALDGLEETGRFSSFLSR | |
| signal | SDSYGYLLLQELQMKDSASYFCAFAYGQNFVFG | |
| peptide | PGTRLSVLP | |
| (IMGT) | | |
| Vα | MXGAFLLYVSMKMGGTAGQSLEQPSEVTAVEGA | 1326 |
| | IVQINCTYQTSGFYGLSWYQQHDGGAPTFLSYN | |
| | ALDGLEETGRESSFLSRSDSYGYLLLQELQMKD | |
| | SASYFCAFAYGQNFVFGPGTRLSVLP | |
| | (X = any amino acid) | 1327 |
| α chain | MWGAFLLYVSMKMGGTAGQSLEQPSEVTAVEGA | 1328 |
| with WT | IVQINCTYQTSGFYGLSWYQQHDGGAPTFLSYN | |
| signal | ALDGLEETGRFSSFLSRSDSYGYLLLQELQMKD | |
| peptide, | SASYFCAFAYGQNFVFGPGTRLSVLPNIQNPEP | |
| Cα (sub- | AVYQLKDPRSQDSTLCLFTDFDSQINVPKTMES | |
| stituted) | GTFITDKCVLDMKAMDSKSNGAIAWSNQTSFTC | |
| | QDIFKETNATYPSSDVPCDATLTEKSFETDMNL | |
| | NFQNLLVIVLRILLLKVAGFNLLMTLRLWSS | |
| α chain | MAGAFLLYVSMKMGGTAGQSLEQPSEVTAVEGA | 1329 |
| with al- | IVQINCTYQTSGFYGLSWYQQHDGGAPTFLSYN | |
| ternative | ALDGLEETGRESSFLSRSDSYGYLLLQELQMKD | |
| signal | SASYFCAFAYGQNFVFGPGTRLSVLPNIQNPEP | |
| peptide, | AVYQLKDPRSQDSTLCLFTDFDSQINVPKTMES | |
| Cα (sub- | GTFITDKCVLDMKAMDSKSNGAIAWSNQTSFTC | |
| stituted) | QDIFKETNATYPSSDVPCDATLTEKSFETDMNL | |
| | NFQNLLVIVLRILLLKVAGFNLLMTLRLWSS | |
| α chain | MHGAFLLYVSMKMGGTAGQSLEQPSEVTAVEGA | 1330 |
| with al- | IVQINCTYQTSGFYGLSWYQQHDGGAPTFLSYN | |
| ternative | ALDGLEETGRESSFLSRSDSYGYLLLQELQMKD | |
| signal | SASYFCAFAYGQNFVFGPGTRLSVLPNIQNPEP | |
| peptide, | AVYQLKDPRSQDSTLCLFTDFDSQINVPKTMES | |
| Cα (sub- | GTFITDKCVLDMKAMDSKSNGAIAWSNQTSFTC | |
| stituted) | QDIFKETNATYPSSDVPCDATLTEKSFETDMNL | |
| | NFQNLLVIVLRILLLKVAGFNLLMTLRLWSS | |
| CDR1ß | SGDLS | 2321 |
| CDR2ß | YYNGEE | 2322 |
| CDR3ß | ASSPLGDSGNTIY | 2323 |
| Vß | GVTQTPKHLITATGQRVTLRCSPRSGDLSVYWY | 2324 |
| without | QQSLDQGLQFLIQYYNGEERAKGNILERFSAQQ | |
| signal | FPDLHSELNLSSLELGDSALYFCASSPLGDSGN | |
| peptide | TIYFGEGSWLTVV | |
| (SignalP) | | |
| Vß | DSGVTQTPKHLITATGQRVTLRCSPRSGDLSVY | 2325 |
| without | WYQQSLDQGLQFLIQYYNGEERAKGNILERFSA | |
| signal | QQFPDLHSELNLSSLELGDSALYFCASSPLGDS | |
| peptide | GNTIYFGEGSWLTVV | |
| (IMGT) | | |
| Vß | MXFRLLCCVAFCLLGAGPVDSGVTQTPKHLITA | 2326 |
| | TGQRVTLRCSPRSGDLSVYWYQQSLDQGLQFLI | |
| | QYYNGEERAKGNILERFSAQQFPDLHSELNLSS | |
| | LELGDSALYFCASSPLGDSGNTIYFGEGSWLTV | |
| | V | |
| | (X = any amino acid) | 2327 |
| ß chain | MGFRLLCCVAFCLLGAGPVDSGVTQTPKHLITA | 2328 |
| with WT | TGQRVTLRCSPRSGDLSVYWYQQSLDQGLQFLI | |
| signal | QYYNGEERAKGNILERFSAQQFPDLHSELNLSS | |
| peptide, | LELGDSALYFCASSPLGDSGNTIYFGEGSWLTV | |
| Cß (sub- | VEDLRNVTPPKVSLFEPSKAEIANKQKATLVCL | |
| stituted) | ARGFFPDHVELSWWVNGKEVHSGVCTDPQAYKE | |
| | SNYSYCLSSRLRVSATFWHNPRNHFRCQVQFHG | |
| | LSEEDKWPEGSPKPVTQNISAEAWGRADCGITS | |
| | ASYQQGVLSATILYEILLGKATLYAVLVSTLVV | |
| | MAMVKRKNS | |
| ß chain | MAFRLLCCVAFCLLGAGPVDSGVTQTPKHLITA | 2329 |
| with al- | TGQRVTLRCSPRSGDLSVYWYQQSLDQGLQFLI | |
| ternative | QYYNGEERAKGNILERFSAQQFPDLHSELNLSS | |
| signal | LELGDSALYFCASSPLGDSGNTIYFGEGSWLTV | |
| peptide, | VEDLRNVTPPKVSLFEPSKAEIANKQKATLVCL | |
| Cß (sub- | ARGFFPDHVELSWWVNGKEVHSGVCTDPQAYKE | |
| stituted) | SNYSYCLSSRLRVSATFWHNPRNHFRCQVQFHG | |
| | LSEEDKWPEGSPKPVTQNISAEAWGRADCGITS | |
| | ASYQQGVLSATILYEILLGKATLYAVLVSTLVV | |
| | MAMVKRKNS | |
| ß chain | MHFRLLCCVAFCLLGAGPVDSGVTQTPKHLITA | 2330 |
| with al- | TGQRVTLRCSPRSGDLSVYWYQQSLDQGLQFLI | |
| ternative | QYYNGEERAKGNILERFSAQQFPDLHSELNLSS | |
| signal | LELGDSALYFCASSPLGDSGNTIYFGEGSWLTV | |
| peptide, | VEDLRNVTPPKVSLFEPSKAEIANKQKATLVCL | |
| Cß (sub- | ARGFFPDHVELSWWVNGKEVHSGVCTDPQAYKE | |
| stituted) | SNYSYCLSSRLRVSATFWHNPRNHFRCQVQFHG | |
| | LSEEDKWPEGSPKPVTQNISAEAWGRADCGITS | |
| | ASYQQGVLSATILYEILLGKATLYAVLVSTLVV | |
| | MAMVKRKNS | |

In some embodiments, TCR034 interacts with and/or is specific for p53. In some embodiments, the peptide is from a neoantigen of p53. In some embodiments, the neoantigen has the amino acid change R248Q relative to the wild type p53 sequence. In some embodiments, TCR034 interacts with the neoantigen in the context of HLA-A*02:01, as described in

TABLE 6AH
Amino acid sequences of TCR034.

| | | SEQ |
|---|---|---|
| Descrip- | | ID |
| tion | Sequence | NO: |
| CDR1α | TSINN | 1331 |
| CDR2α | IRSNERE | 1332 |
| CDR3α | ATDAWNNDMR | 1333 |
| Vα | QQGEEDPQALSIQEGENATMNCSYKTSINNLQW | 1334 |
| without | YRQNSGRGLVHLILIRSNEREKHSGRLRVTLDT | |
| signal | SKKSSSLLITASRAADTASYFCATDAWNNDMRF | |
| peptide | GAGTRLTVKP | |
| (SignalP) | | |
| Vα | SQQGEEDPQALSIQEGENATMNCSYKTSINNLQ | 1335 |
| without | WYRQNSGRGLVHLILIRSNEREKHSGRLRVTLD | |
| signal | TSKKSSSLLITASRAADTASYFCATDAWNNDMR | |
| peptide | FGAGTRLTVKP | |
| (IMGT) | | |
| Vα | MXTLLGVSLVILWLQLARVNSQQGEEDPQALSI | 1336 |
| | QEGENATMNCSYKTSINNLQWYRQNSGRGLVHL | |
| | ILIRSNEREKHSGRLRVTLDTSKKSSSLLITAS | |
| | RAADTASYFCATDAWNNDMRFGAGTRLTVKP | |
| | (X = any amino acid) | 1337 |
| α chain | METLLGVSLVILWLQLARVNSQQGEEDPQALSI | 1338 |
| with WT | QEGENATMNCSYKTSINNLQWYRQNSGRGLVHL | |
| signal | ILIRSNEREKHSGRLRVTLDTSKKSSSLLITAS | |
| peptide, | RAADTASYFCATDAWNNDMRFGAGTRLTVKPNI | |
| Cα (sub- | QNPEPAVYQLKDPRSQDSTLCLFTDFDSQINVP | |
| stituted) | KTMESGTFITDKCVLDMKAMDSKSNGAIAWSNQ | |
| | TSFTCQDIFKETNATYPSSDVPCDATLTEKSFE | |
| | TDMNLNFQNLLVIVLRILLLKVAGFNLLMTLRL | |
| | WSS | |
| α chain | MATLLGVSLVILWLQLARVNSQQGEEDPQALSI | 1339 |
| with al- | QEGENATMNCSYKTSINNLQWYRQNSGRGLVHL | |
| ternative | ILIRSNEREKHSGRLRVTLDTSKKSSSLLITAS | |
| signal | RAADTASYFCATDAWNNDMRFGAGTRLTVKPNI | |
| peptide, | QNPEPAVYQLKDPRSQDSTLCLFTDFDSQINVP | |
| Cα (sub- | KTMESGTFITDKCVLDMKAMDSKSNGAIAWSNQ | |
| stituted) | TSFTCQDIFKETNATYPSSDVPCDATLTEKSFE | |
| | TDMNLNFQNLLVIVLRILLLKVAGFNLLMTLRL | |
| | WSS | |
| α chain | MHTLLGVSLVILWLQLARVNSQQGEEDPQALSI | 1340 |
| with al- | QEGENATMNCSYKTSINNLQWYRQNSGRGLVHL | |
| ternative | ILIRSNEREKHSGRLRVTLDTSKKSSSLLITAS | |
| signal | RAADTASYFCATDAWNNDMRFGAGTRLTVKPNI | |
| peptide, | QNPEPAVYQLKDPRSQDSTLCLFTDFDSQINVP | |
| Cα (sub- | KTMESGTFITDKCVLDMKAMDSKSNGAIAWSNQ | |
| stituted) | TSFTCQDIFKETNATYPSSDVPCDATLTEKSFE | |
| | TDMNLNFQNLLVIVLRILLLKVAGFNLLMTLRL | |
| | WSS | |
| CDR1ß | MNHNS | 2331 |
| CDR2ß | SASEG | 2332 |
| CDR3ß | ASSESQGNTEAF | 2333 |
| Vß | GVTQTPKFQVLKTGQSMTLQCAQDMNHNSMYWY | 2334 |
| without | RQDPGMGLRLIYYSASEGTTDKGEVPNGYNVSR | |
| signal | LNKREFSLRLESAAPSQTSVYFCASSESQGNTE | |
| peptide | AFFGQGTRLTVV | |
| (SignalP) | | |
| Vß | NAGVTQTPKFQVLKTGQSMTLQCAQDMNHNSMY | 2335 |
| without | WYRQDPGMGLRLIYYSASEGTTDKGEVPNGYNV | |
| signal | SRLNKREFSLRLESAAPSQTSVYFCASSESQGN | |
| peptide | TEAFFGQGTRLTVV | |
| (IMGT) | | |
| Vß | MXIGLLCCVAFSLLWASPVNAGVTQTPKFQVLK | 2336 |
| | TGQSMTLQCAQDMNHNSMYWYRQDPGMGLRLIY | |
| | YSASEGTTDKGEVPNGYNVSRLNKREFSLRLES | |
| | AAPSQTSVYFCASSESQGNTEAFFGQGTRLTVV | |
| | (X = any amino acid) | 2337 |
| ß chain | MSIGLLCCVAFSLLWASPVNAGVTQTPKFQVLK | 2338 |
| with WT | TGQSMTLQCAQDMNHNSMYWYRQDPGMGLRLIY | |
| signal | YSASEGTTDKGEVPNGYNVSRLNKREFSLRLES | |
| peptide, | AAPSQTSVYFCASSESQGNTEAFFGQGTRLTVV | |
| Cß (sub- | EDLRNVTPPKVSLFEPSKAEIANKQKATLVCLA | |
| stituted) | RGFFPDHVELSWWVNGKEVHSGVCTDPQAYKES | |
| | NYSYCLSSRLRVSATFWHNPRNHFRCQVQFHGL | |
| | SEEDKWPEGSPKPVTQNISAEAWGRADCGITSA | |
| | SYQQGVLSATILYEILLGKATLYAVLVSTLVVM | |
| | AMVKRKNS | |
| ß chain | MAIGLLCCVAFSLLWASPVNAGVTQTPKFQVLK | 2339 |
| with al- | TGQSMTLQCAQDMNHNSMYWYRQDPGMGLRLIY | |
| ternative | YSASEGTTDKGEVPNGYNVSRLNKREFSLRLES | |
| signal | AAPSQTSVYFCASSESQGNTEAFFGQGTRLTVV | |
| peptide, | EDLRNVTPPKVSLFEPSKAEIANKQKATLVCLA | |
| Cß (sub- | RGFFPDHVELSWWVNGKEVHSGVCTDPQAYKES | |
| stituted) | NYSYCLSSRLRVSATFWHNPRNHFRCQVQFHGL | |
| | SEEDKWPEGSPKPVTQNISAEAWGRADCGITSA | |
| | SYQQGVLSATILYEILLGKATLYAVLVSTLVVM | |
| | AMVKRKNS | |
| ß chain | MHIGLLCCVAFSLLWASPVNAGVTQTPKFQVLK | 2340 |
| with WT | TGQSMTLQCAQDMNHNSMYWYRQDPGMGLRLIY | |
| signal | YSASEGTTDKGEVPNGYNVSRLNKREFSLRLES | |
| peptide, | AAPSQTSVYFCASSESQGNTEAFFGQGTRLTVV | |
| Cß (sub- | EDLRNVTPPKVSLFEPSKAEIANKQKATLVCLA | |
| stituted) | RGFFPDHVELSWWVNGKEVHSGVCTDPQAYKES | |
| | NYSYCLSSRLRVSATFWHNPRNHFRCQVQFHGL | |
| | SEEDKWPEGSPKPVTQNISAEAWGRADCGITSA | |
| | SYQQGVLSATILYEILLGKATLYAVLVSTLVVM | |
| | AMVKRKNS | |

In some embodiments, TCR034 interacts with and/or is specific for p53. In some embodiments, the peptide is from a neoantigen of p53. In some embodiments, the neoantigen has the amino acid change R248Q relative to the wild type p53 sequence. In some embodiments, TCR034 interacts with the neoantigen in the context of HLA-A*02:01, as described in International Publication No. WO 2019/067243, incorporated herein by reference in its entirety.

TABLE 6AI
Amino acid sequences of TCR035.

| | | SEQ |
|---|---|---|
| Descrip- | | ID |
| tion | Sequence | NO: |
| CDR1α | VSPFSN | 1341 |
| CDR2α | MTFSENT | 1342 |
| CDR3α | VVSSYKII | 1343 |
| Vα | KNQVEQSPQSLIILEGKNCTLQCNYTVSPFSNL | 1344 |
| without | RWYKQDTGRGPVSLTIMTFSENTKSNGRYTATL | |
| signal | DADTKQSSLHITASQLSDSASYICVVSSYKIIF | |
| peptide | GTGTRLHVFP | |
| (SignalP) | | |
| Vα | KNQVEQSPQSLIILEGKNCTLQCNYTVSPFSNL | 1345 |
| without | RWYKQDTGRGPVSLTIMTFSENTKSNGRYTATL | |
| signal | DADTKQSSLHITASQLSDSASYICVVSSYKIIF | |
| peptide | GTGTRLHVFP | |
| (IMGT) | | |
| Vα | MXKHLTTFLVILWLYFYRGNGKNQVEQSPQSLI | 1346 |
| | ILEGKNCTLQCNYTVSPFSNLRWYKQDTGRGPV | |
| | SLTIMTFSENTKSNGRYTATLDADTKQSSLHIT | |
| | ASQLSDSASYICVVSSYKIIFGTGTRLHVFP | |
| | (X = any amino acid) | 1347 |
| α chain | MKKHLTTFLVILWLYFYRGNGKNQVEQSPQSLI | 1348 |
| with WT | ILEGKNCTLQCNYTVSPFSNLRWYKQDTGRGPV | |
| signal | SLTIMTFSENTKSNGRYTATLDADTKQSSLHIT | |
| peptide, | ASQLSDSASYICVVSSYKIIFGTGTRLHVFPNI | |
| Cα (sub- | QNPEPAVYQLKDPRSQDSTLCLFTDFDSQINVP | |
| stituted) | KTMESGTFITDKCVLDMKAMDSKSNGAIAWSNQ | |
| | TSFTCQDIFKETNATYPSSDVPCDATLTEKSFE | |
| | TDMNLNFQNLLVIVLRILLLKVAGFNLLMTLRL | |
| | WSS | |
| α chain | MAKHLTTFLVILWLYFYRGNGKNQVEQSPQSLI | 1349 |
| with al- | ILEGKNCTLQCNYTVSPFSNLRWYKQDTGRGPV | |
| ternative | SLTIMTFSENTKSNGRYTATLDADTKQSSLHIT | |
| signal | ASQLSDSASYICVVSSYKIIFGTGTRLHVFPNI | |
| peptide, | QNPEPAVYQLKDPRSQDSTLCLFTDFDSQINVP | |
| Cα (sub- | KTMESGTFITDKCVLDMKAMDSKSNGAIAWSNQ | |
| stituted) | TSFTCQDIFKETNATYPSSDVPCDATLTEKSFE | |
| | TDMNLNFQNLLVIVLRILLLKVAGFNLLMTLRL | |
| | WSS | |
| α chain | MHKHLTTFLVILWLYFYRGNGKNQVEQSPQSLI | 1350 |
| with al- | ILEGKNCTLQCNYTVSPFSNLRWYKQDTGRGPV | |
| ternative | SLTIMTFSENTKSNGRYTATLDADTKQSSLHIT | |
| signal | ASQLSDSASYICVVSSYKIIFGTGTRLHVFPNI | |
| peptide, | QNPEPAVYQLKDPRSQDSTLCLFTDFDSQINVP | |
| Cα (sub- | KTMESGTFITDKCVLDMKAMDSKSNGAIAWSNQ | |
| stituted) | TSFTCQDIFKETNATYPSSDVPCDATLTEKSFE | |
| | TDMNLNFQNLLVIVLRILLLKVAGFNLLMTLRL | |
| | WSS | |
| CDR1ß | SGHTA | 2341 |
| CDR2ß | FQGNSA | 2342 |
| CDR3ß | ASSPIQGENSPLH | 2343 |
| Vß | GVSQSPSNKVTEKGKDVELRCDPISGHTALYWY | 2344 |
| without | RQRLGQGLEFLIYFQGNSAPDKSGLPSDRFSAE | |
| signal | RTGESVSTLTIQRTQQEDSAVYLCASSPIQGEN | |
| peptide | SPLHFGNGTRLTVT | |
| (SignalP) | | |
| Vß | GAGVSQSPSNKVTEKGKDVELRCDPISGHTALY | 2345 |
| without | WYRQRLGQGLEFLIYFQGNSAPDKSGLPSDRES | |
| signal | AERTGESVSTLTIQRTQQEDSAVYLCASSPIQG | |
| peptide | ENSPLHFGNGTRLTVT | |
| (IMGT) | | |
| Vß | MXTRLLFWVAFCLLGAYHTGAGVSQSPSNKVTE | 2346 |
| | KGKDVELRCDPISGHTALYWYRQRLGQGLEFLI | |
| | YFQGNSAPDKSGLPSDRESAERTGESVSTLTIQ | |
| | RTQQEDSAVYLCASSPIQGENSPLHFGNGTRLT | |
| | VT | |
| | (X = any amino acid) | 2347 |
| ß chain | MGTRLLFWVAFCLLGAYHTGAGVSQSPSNKVTE | 2348 |
| with WT | KGKDVELRCDPISGHTALYWYRQRLGQGLEFLI | |
| signal | YFQGNSAPDKSGLPSDRFSAERTGESVSTLTIQ | |
| peptide, | RTQQEDSAVYLCASSPIQGENSPLHFGNGTRLT | |
| Cß (sub- | VTEDLRNVTPPKVSLFEPSKAEIANKQKATLVC | |
| stituted) | LARGFFPDHVELSWWVNGKEVHSGVCTDPQAYK | |
| | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MATRLLFWVAFCLLGAYHTGAGVSQSPSNKVTE | 2349 |
| with al- | KGKDVELRCDPISGHTALYWYRQRLGQGLEFLI | |
| ternative | YFQGNSAPDKSGLPSDRFSAERTGESVSTLTIQ | |
| signal | RTQQEDSAVYLCASSPIQGENSPLHFGNGTRLT | |
| peptide, | VTEDLRNVTPPKVSLFEPSKAEIANKQKATLVC | |
| Cß (sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |
| ß chain | MHTRLLFWVAFCLLGAYHTGAGVSQSPSNKVTE | 2350 |
| with al- | KGKDVELRCDPISGHTALYWYRQRLGQGLEFLI | |
| ternative | YFQGNSAPDKSGLPSDRFSAERTGESVSTLTIQ | |
| signal | RTQQEDSAVYLCASSPIQGENSPLHFGNGTRLT | |
| peptide, | VTEDLRNVTPPKVSLFEPSKAEIANKQKATLVC | |
| Cß (sub- | LARGFFPDHVELSWWVNGKEVHSGVCTDPQAYK | |
| stituted) | ESNYSYCLSSRLRVSATFWHNPRNHFRCQVQFH | |
| | GLSEEDKWPEGSPKPVTQNISAEAWGRADCGIT | |
| | SASYQQGVLSATILYEILLGKATLYAVLVSTLV | |
| | VMAMVKRKNS | |

In some embodiments, TCR035 interacts with and/or is specific for p53. In some embodiments, the peptide is from a neoantigen of p53. In some embodiments, the neoantigen has the amino acid change R248Q relative to the wild type p53 sequence. In some embodiments, TCR035 interacts with the neoantigen in the context of HLA-A*02:01, as described in

TABLE 6AJ
Amino acid sequences of TCR036.

| | | SEQ |
|---|---|---|
| | | ID |
| Description | Sequence | NO: |
| CDR1α | VSPFSN | 1351 |
| CDR2α | MTFSENT | 1352 |
| CDR3α | VVSSYKLI | 1353 |
| Vα without signal | KNQVEQSPQSLIILEGKNCTLQCNYTVSPFSNLRWYKQDTGRGPVSLTIMTF | 1354 |
| peptide (SignalP) | SENTKSNGRYTATLDADTKQSSLHITASQLSDSASYICVVSSYKLIFGTGTR | |
| | LQVFP | |
| Vα without signal | KNQVEQSPQSLIILEGKNCTLQCNYTVSPFSNLRWYKQDTGRGPVSLTIMTF | 1355 |
| peptide (IMGT) | SENTKSNGRYTATLDADTKQSSLHITASQLSDSASYICVVSSYKLIFGTGTR | |
| | LQVFP | |
| Vα | MXKHLTTFLVILWLYFYRGNGKNQVEQSPQSLIILEGKNCTLQCNYTVSPFS | 1356 |
| | NLRWYKQDTGRGPVSLTIMTFSENTKSNGRYTATLDADTKQSSLHITASQLS | 1357 |
| | DSASYICVVSSYKLIFGTGTRLQVFP | |
| | (X = any amino acid) | |
| α chain with WT signal | MKKHLTTFLVILWLYFYRGNGKNQVEQSPQSLIILEGKNCTLQCNYTVSPFS | 1358 |
| peptide, Cα | NLRWYKQDTGRGPVSLTIMTFSENTKSNGRYTATLDADTKQSSLHITASQLS | |
| (substituted) | DSASYICVVSSYKLIFGTGTRLQVFPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| α | MAKHLTTFLVILWLYFYRGNGKNQVEQSPQSLIILEGKNCTLQCNYTVSPFS | 1359 |
| chain with | NLRWYKQDTGRGPVSLTIMTFSENTKSNGRYTATLDADTKQSSLHITASQLS | |
| alternative signal | DSASYICVVSSYKLIFGTGTRLQVFPNIQNPEPAVYQLKDPRSQDSTLCLFT | |
| peptide, Cα | DFDSQINVPKTMESGTFITDKCVLDMKAMDSKSNGAIAWSNQTSFTCQDIFK | |
| (substituted) | ETNATYPSSDVPCDATLTEKSFETDMNLNFQNLLVIVLRILLLKVAGENLLM | |
| | TLRLWSS | |
| α | MHKHLTTFLVILWLYFYRGNGKNQVEQSPQSLIILEGKNCTLQCNYTVSPFS | 1360 |
| chain with | NLRWYKQDTGRGPVSLTIMTFSENTKSNGRYTATLDADTKQSSLHITASQLS | |
| alternative signal | DSASYICVVSSYKLIFGTGTRLQVFPNIQNPEPAVYQLKDPRSQDSTLCLFT | |
| peptide, Cα | DFDSQINVPKTMESGTFITDKCVLDMKAMDSKSNGAIAWSNQTSFTCQDIFK | |
| (substituted) | ETNATYPSSDVPCDATLTEKSFETDMNLNFQNLLVIVLRILLLKVAGENLLM | |
| | TLRLWSS | |
| CDR1β | SGHTA | 2351 |
| CDR2β | FQGNSA | 2352 |
| CDR3β | ASSPIQGENSPLH | 2353 |
| Vβ without signal | GVSQSPSNKVTEKGKDVELRCDPISGHTALYWYRQRLGQGLEFLIYFQGNSA | 2354 |
| peptide (SignalP) | PDKSGLPSDRFSAERTGESVSTLTIQRTQQEDSAVYLCASSPIQGENSPLHF | |
| | GNGTRLTVT | |
| Vβ without signal | GAGVSQSPSNKVTEKGKDVELRCDPISGHTALYWYRQRLGQGLEFLIYFQGN | 2355 |
| peptide (IMGT) | SAPDKSGLPSDRFSAERTGESVSTLTIQRTQQEDSAVYLCASSPIQGENSPL | |
| | HFGNGTRLTVT | |
| Vβ | MXTRLLFWVAFCLLGAYHTGAGVSQSPSNKVTEKGKDVELRCDPISGHTALY | 2356 |
| | WYRQRLGQGLEFLIYFQGNSAPDKSGLPSDRFSAERTGESVSTLTIQRTQQE | 2357 |
| | DSAVYLCASSPIQGENSPLHFGNGTRLTVT | |
| | (X = any amino acid) | |
| β chain with WT signal | MGTRLLFWVAFCLLGAYHTGAGVSQSPSNKVTEKGKDVELRCDPISGHTALY | 2358 |
| peptide, Cβ | WYRQRLGQGLEFLIYFQGNSAPDKSGLPSDRESAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLFWVAFCLLGAYHTGAGVSQSPSNKVTEKGKDVELRCDPISGHTALY | 2359 |
| signal peptide, Cβ | WYRQRLGQGLEFLIYFQGNSAPDKSGLPSDRESAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLFWVAFCLLGAYHTGAGVSQSPSNKVTEKGKDVELRCDPISGHTALY | 2360 |
| signal peptide, Cβ | WYRQRLGQGLEFLIYFQGNSAPDKSGLPSDRESAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR036 interacts with and/or is specific for p53. In some embodiments, the peptide is from a neoantigen of p53. In some embodiments, the neoantigen has the amino acid change R248Q relative to the wild type p53 sequence. In some embodiments, TCR036 interacts with the neoantigen in the context of HLA-A*02:01, as described in International Publication No. WO 2019/067243, incorporated herein by reference in its entirety.

TABLE 6AK
Amino acid sequences of TCR037.

| | | SEQ |
|---|---|---|
| | | ID |
| Description | Sequence | NO: |
| CDR1α | SSVSVY | 1361 |
| CDR2α | YLSGSTLV | 1362 |
| CDR3α | AVSKGTGAQKLV | 1363 |
| Vα without signal | QSVTQLDSQVPVFEEAPVELRCNYSSSVSVYLFWYVQYPNQGLQLLLKYLSG | 1364 |
| peptide (SignalP) | STLVESINGFEAEFNKSQTSFHLRKPSVHISDTAEYFCAVSKGTGAQKLVFG | |
| | QGTRLTINP | |
| Vα without signal | AQSVTQLDSQVPVFEEAPVELRCNYSSSVSVYLFWYVQYPNQGLQLLLKYLS | 1365 |
| peptide (IMGT) | GSTLVESINGFEAEFNKSQTSFHLRKPSVHISDTAEYFCAVSKGTGAQKLVF | |
| | GQGTRLTINP | |
| Vα | MXLLLVPAFQVIFTLGGTRAQSVTQLDSQVPVFEEAPVELRCNYSSSVSVYL | 1366 |
| | FWYVQYPNQGLQLLLKYLSGSTLVESINGFEAEFNKSQTSFHLRKPSVHISD | 1367 |
| | TAEYFCAVSKGTGAQKLVFGQGTRLTINP | |
| | (X = any amino acid) | |
| α chain with WT signal | MLLLLVPAFQVIFTLGGTRAQSVTQLDSQVPVFEEAPVELRCNYSSSVSVYL | 1368 |
| peptide, Cα | FWYVQYPNQGLQLLLKYLSGSTLVESINGFEAEFNKSQTSFHLRKPSVHISD | |
| (substituted) | TAEYFCAVSKGTGAQKLVFGQGTRLTINPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MALLLVPAFQVIFTLGGTRAQSVTQLDSQVPVFEEAPVELRCNYSSSVSVYL | 1369 |
| alternative signal | FWYVQYPNQGLQLLLKYLSGSTLVESINGFEAEFNKSQTSFHLRKPSVHISD | |
| peptide, Cα | TAEYFCAVSKGTGAQKLVFGQGTRLTINPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MHLLLVPAFQVIFTLGGTRAQSVTQLDSQVPVFEEAPVELRCNYSSSVSVYL | 1370 |
| alternative signal | FWYVQYPNQGLQLLLKYLSGSTLVESINGFEAEFNKSQTSFHLRKPSVHISD | |
| peptide, Cα | TAEYFCAVSKGTGAQKLVFGQGTRLTINPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| CDR1β | LNHDA | 2361 |
| CDR2β | SQIVND | 2362 |
| CDR3β | ASEAF | 2363 |
| Vβ without signal | GITQSPKYLFRKEGQNVTLSCEQNLNHDAMYWYRQDPGQGLRLIYYSQIVND | 2364 |
| peptide (SignalP) | FQKGDIAEGYSVSREKKESFPLIVTSAQKNPTASYLCASEAFFGQGTRLTVV | 2365 |
| Vβ without signal | DGGITQSPKYLFRKEGQNVTLSCEQNLNHDAMYWYRQDPGQGLRLIYYSQIV | |
| peptide (IMGT) | NDFQKGDIAEGYSVSREKKESFPLIVTSAQKNPTASYLCASEAFFGQGTRLT | |
| | VV | |
| Vβ | MXNQVLCCVVLCFLGANTVDGGITQSPKYLFRKEGQNVTLSCEQNLNHDAMY | 2366 |
| | WYRQDPGQGLRLIYYSQIVNDFQKGDIAEGYSVSREKKESFPLIVTSAQKNP | 2367 |
| | TASYLCASEAFFGQGTRLTVV | |
| | (X = any amino acid) | |
| β chain with WT signal | MSNQVLCCVVLCFLGANTVDGGITQSPKYLFRKEGQNVTLSCEQNLNHDAMY | 2368 |
| peptide, Cβ | WYRQDPGQGLRLIYYSQIVNDFQKGDIAEGYSVSREKKESFPLIVTSAQKNP | |
| (substituted) | TASYLCASEAFFGQGTRLTVVEDLRNVTPPKVSLFEPSKAEIANKQKATLVC | |
| | LARGFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSASYQ | |
| | QGVLSATILYEILLGKATLYAVLVSTLVVMAMVKRKNS | |
| β chain with alternative | MANQVLCCVVLCFLGANTVDGGITQSPKYLFRKEGQNVTLSCEQNLNHDAMY | 2369 |
| signal peptide, Cβ | WYRQDPGQGLRLIYYSQIVNDFQKGDIAEGYSVSREKKESFPLIVTSAQKNP | |
| (substituted) | TASYLCASEAFFGQGTRLTVVEDLRNVTPPKVSLFEPSKAEIANKQKATLVC | |
| | LARGFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSASYQ | |
| | QGVLSATILYEILLGKATLYAVLVSTLVVMAMVKRKNS | |
| β chain with alternative | MHNQVLCCVVLCFLGANTVDGGITQSPKYLFRKEGQNVTLSCEQNLNHDAMY | 2370 |
| signal peptide, Cβ | WYRQDPGQGLRLIYYSQIVNDFQKGDIAEGYSVSREKKESFPLIVTSAQKNP | |
| (substituted) | TASYLCASEAFFGQGTRLTVVEDLRNVTPPKVSLFEPSKAEIANKQKATLVC | |
| | LARGFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSASYQ | |
| | QGVLSATILYEILLGKATLYAVLVSTLVVMAMVKRKNS | |

In some embodiments, TCR037 interacts with and/or is specific for p53. In some embodiments, the peptide is from a neoantigen of p53. In some embodiments, the neoantigen has the amino acid change R248Q relative to the wild type p53 sequence. In some embodiments, TCR037 interacts with the neoantigen in the context of HLA-A*02:01, as described in

TABLE 6AL
Amino acid sequences of TCR038.

| | | SEQ |
|---|---|---|
| | | ID |
| Description | Sequence | NO: |
| CDR1α | TISGTDY | 1371 |
| CDR2α | GLTSN | 1372 |
| CDR3α | ILASGAGSYQLT | 1373 |
| Vα without signal | KTTQPNSMESNEEEPVHLPCNHSTISGTDYIHWYRQLPSQGPEYVIHGLTSN | 1374 |
| peptide (SignalP) | VNNRMASLAIAEDRKSSTLILHRATLRDAAVYYCILASGAGSYQLTFGKGTK | |
| | LSVIP | |
| Vα without signal | DAKTTQPNSMESNEEEPVHLPCNHSTISGTDYIHWYRQLPSQGPEYVIHGLT | 1375 |
| peptide (IMGT) | SNVNNRMASLAIAEDRKSSTLILHRATLRDAAVYYCILASGAGSYQLTFGKG | |
| | TKLSVIP | |
| Vα | MXLVTSITVLLSLGIMGDAKTTQPNSMESNEEEPVHLPCNHSTISGTDYIHW | 1376 |
| | YRQLPSQGPEYVIHGLTSNVNNRMASLAIAEDRKSSTLILHRATLRDAAVYY | 1377 |
| | CILASGAGSYQLTFGKGTKLSVIP | |
| | (X = any amino acid) | |
| α chain with WT signal | MKLVTSITVLLSLGIMGDAKTTQPNSMESNEEEPVHLPCNHSTISGTDYIHW | 1378 |
| peptide, Cα | YRQLPSQGPEYVIHGLTSNVNNRMASLAIAEDRKSSTLILHRATLRDAAVYY | |
| (substituted) | CILASGAGSYQLTFGKGTKLSVIPNIQNPEPAVYQLKDPRSQDSTLCLFTDF | |
| | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α | MALVTSITVLLSLGIMGDAKTTQPNSMESNEEEPVHLPCNHSTISGTDYIHW | 1379 |
| chain with | YRQLPSQGPEYVIHGLTSNVNNRMASLAIAEDRKSSTLILHRATLRDAAVYY | |
| alternative signal | CILASGAGSYQLTFGKGTKLSVIPNIQNPEPAVYQLKDPRSQDSTLCLFTDF | |
| peptide, Cα | DSQINVPKTMESGTFITDKCVLDMKAMDSKSNGAIAWSNQTSFTCQDIFKET | |
| (substituted) | NATYPSSDVPCDATLTEKSFETDMNLNFQNLLVIVLRILLLKVAGENLLMTL | |
| | RLWSS | |
| α chain with | MHLVTSITVLLSLGIMGDAKTTQPNSMESNEEEPVHLPCNHSTISGTDYIHW | 1380 |
| alternative signal | YRQLPSQGPEYVIHGLTSNVNNRMASLAIAEDRKSSTLILHRATLRDAAVYY | |
| peptide, Cα | CILASGAGSYQLTFGKGTKLSVIPNIQNPEPAVYQLKDPRSQDSTLCLFTDE | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| CDR1β | LGHD | 2371 |
| CDR2β | YNNKEL | 2372 |
| CDR3β | ASRTIGYNTEAF | 2373 |
| Vβ without signal | QTPKYLVTQMGNDKSIKCEQNLGHDTMYWYKQDSKKFLKIMFSYNNKELIIN | 2374 |
| peptide (SignalP) | ETVPNRFSPKSPDKAHLNLHINSLELGDSAVYFCASRTIGYNTEAFFGQGTR | |
| | LTVV | |
| Vβ without signal | DTAVSQTPKYLVTQMGNDKSIKCEQNLGHDTMYWYKQDSKKFLKIMFSYNNK | 2375 |
| peptide (IMGT) | ELIINETVPNRFSPKSPDKAHLNLHINSLELGDSAVYFCASRTIGYNTEAFF | |
| | GQGTRLTVV | |
| Vβ | MXCRLLCCVVFCLLQAGPLDTAVSQTPKYLVTQMGNDKSIKCEQNLGHDTMY | 2376 |
| | WYKQDSKKFLKIMFSYNNKELIINETVPNRESPKSPDKAHLNLHINSLELGD | 2377 |
| | SAVYFCASRTIGYNTEAFFGQGTRLTVV | |
| | (X = any amino acid) | |
| β chain with WT signal | MGCRLLCCVVFCLLQAGPLDTAVSQTPKYLVTQMGNDKSIKCEQNLGHDTMY | 2378 |
| peptide, Cβ | WYKQDSKKFLKIMFSYNNKELIINETVPNRFSPKSPDKAHLNLHINSLELGD | |
| (substituted) | SAVYFCASRTIGYNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MACRLLCCVVFCLLQAGPLDTAVSQTPKYLVTQMGNDKSIKCEQNLGHDTMY | 2379 |
| signal peptide, Cβ | WYKQDSKKFLKIMFSYNNKELIINETVPNRFSPKSPDKAHLNLHINSLELGD | |
| (substituted) | SAVYFCASRTIGYNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHCRLLCCVVFCLLQAGPLDTAVSQTPKYLVTQMGNDKSIKCEQNLGHDTMY | 2380 |
| signal peptide, Cβ | WYKQDSKKFLKIMFSYNNKELIINETVPNRFSPKSPDKAHLNLHINSLELGD | |
| (substituted) | SAVYFCASRTIGYNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR038 interacts with and/or is specific for p53. In some embodiments, the peptide is from a neoantigen of p53. In some embodiments, the neoantigen has the amino acid change R248Q relative to the wild type p53 sequence. In some embodiments, TCR038 interacts with the neoantigen in the context of HLA-A*02:01, as described in International Publication No. WO 2019/067243, incorporated herein by reference in its entirety.

TABLE 6AM
Amino acid sequences of TCR039.

| | | SEQ |
|---|---|---|
| | | ID |
| Description | Sequence | NO: |
| CDR1α | VGISA | 1381 |
| CDR2α | LSSGK | 1382 |
| CDR3α | AALSYNTDKLI | 1383 |
| Vα without signal | AKNEVEQSPQNLTAQEGEFITINCSYSVGISALHWLQQHPGGGIVSLEMLSS | 1384 |
| peptide (SignalP) | GKKKHGRLIATINIQEKHSSLHITASHPRDSAVYICAALSYNTDKLIFGTGT | |
| | RLQVFP | |
| Vα without signal | KNEVEQSPQNLTAQEGEFITINCSYSVGISALHWLQQHPGGGIVSLEMLSSG | 1385 |
| peptide (IMGT) | KKKHGRLIATINIQEKHSSLHITASHPRDSAVYICAALSYNTDKLIFGTGTR | |
| | LQVFP | |
| Vα | MXKIRQFLLAILWLQLSCVSAAKNEVEQSPQNLTAQEGEFITINCSYSVGIS | 1386 |
| | ALHWLQQHPGGGIVSLFMLSSGKKKHGRLIATINIQEKHSSLHITASHPRDS | 1387 |
| | AVYICAALSYNTDKLIFGTGTRLQVFP | |
| | (X = any amino acid) | |
| α chain with WT signal | MKKIRQFLLAILWLQLSCVSAAKNEVEQSPQNLTAQEGEFITINCSYSVGIS | 1388 |
| peptide, Cα | ALHWLQQHPGGGIVSLFMLSSGKKKHGRLIATINIQEKHSSLHITASHPRDS | |
| (substituted) | AVYICAALSYNTDKLIFGTGTRLQVFPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain with | MAKIRQFLLAILWLQLSCVSAAKNEVEQSPQNLTAQEGEFITINCSYSVGIS | 1389 |
| alternative signal | ALHWLQQHPGGGIVSLFMLSSGKKKHGRLIATINIQEKHSSLHITASHPRDS | |
| peptide, Cα | AVYICAALSYNTDKLIFGTGTRLQVFPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain with | MHKIRQFLLAILWLQLSCVSAAKNEVEQSPQNLTAQEGEFITINCSYSVGIS | 1390 |
| alternative signal | ALHWLQQHPGGGIVSLFMLSSGKKKHGRLIATINIQEKHSSLHITASHPRDS | |
| peptide, Cα | AVYICAALSYNTDKLIFGTGTRLQVFPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| CDR1β | SGHTA | 2381 |
| CDR2β | FQGTGA | 2382 |
| CDR3β | ASSLSGLLQETQY | 2383 |
| Vβ without signal | GVSQTPSNKVTEKGKYVELRCDPISGHTALYWYRQSLGQGPEFLIYFQGTGA | 2384 |
| peptide (SignalP) | ADDSGLPNDRFFAVRPEGSVSTLKIQRTERGDSAVYLCASSLSGLLQETQYF | |
| | GPGTRLLVL | |
| Vβ without signal | GAGVSQTPSNKVTEKGKYVELRCDPISGHTALYWYRQSLGQGPEFLIYFQGT | 2385 |
| peptide (IMGT) | GAADDSGLPNDRFFAVRPEGSVSTLKIQRTERGDSAVYLCASSLSGLLQETQ | |
| | YFGPGTRLLVL | |
| Vβ | MXTRLLCWAALCLLGADHTGAGVSQTPSNKVTEKGKYVELRCDPISGHTALY | 2386 |
| | WYRQSLGQGPEFLIYFQGTGAADDSGLPNDRFFAVRPEGSVSTLKIQRTERG | 2387 |
| | DSAVYLCASSLSGLLQETQYFGPGTRLLVL | |
| | (X = any amino acid) | |
| β chain with WT signal | MGTRLLCWAALCLLGADHTGAGVSQTPSNKVTEKGKYVELRCDPISGHTALY | 2388 |
| peptide, Cβ | WYRQSLGQGPEFLIYFQGTGAADDSGLPNDRFFAVRPEGSVSTLKIQRTERG | |
| (substituted) | DSAVYLCASSLSGLLQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGADHTGAGVSQTPSNKVTEKGKYVELRCDPISGHTALY | 2389 |
| signal peptide, Cβ | WYRQSLGQGPEFLIYFQGTGAADDSGLPNDRFFAVRPEGSVSTLKIQRTERG | |
| (substituted) | DSAVYLCASSLSGLLQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGADHTGAGVSQTPSNKVTEKGKYVELRCDPISGHTALY | 2390 |
| signal peptide, Cβ | WYRQSLGQGPEFLIYFQGTGAADDSGLPNDRFFAVRPEGSVSTLKIQRTERG | |
| (substituted) | DSAVYLCASSLSGLLQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR039 interacts with and/or is specific for p53. In some embodiments, the peptide is from a neoantigen of p53. In some embodiments, the neoantigen has the amino acid change R248Q relative to the wild type p53 sequence. In some embodiments, TCR039 interacts with the neoantigen in the context of HLA-A*02:01, as described in

TABLE 6AN
Amino acid sequences of TCR040.

| | | SEQ |
|---|---|---|
| | | ID |
| Description | Sequence | NO: |
| CDR1α | ATGYPS | 1391 |
| CDR2α | ATKADDK | 1392 |
| CDR3α | ALSHTGSSNTGKLI | 1393 |
| Vα without signal | NSVTQMEGPVTLSEEAFLTINCTYTATGYPSLFWYVQYPGEGLQLLLKATKA | 1394 |
| peptide (SignalP) | DDKGSNKGFEATYRKETTSFHLEKGSVQVSDSAVYFCALSHTGSSNTGKLIF | |
| | GQGTRLQVKP | |
| Vα without signal | GNSVTQMEGPVTLSEEAFLTINCTYTATGYPSLFWYVQYPGEGLQLLLKATK | 1395 |
| peptide (IMGT) | ADDKGSNKGFEATYRKETTSFHLEKGSVQVSDSAVYFCALSHTGSSNTGKLI | |
| | FGQGTRLQVKP | |
| Vα | MXYSPGLVSLILLLLGRTRGNSVTQMEGPVTLSEEAFLTINCTYTATGYPSL | 1396 |
| | FWYVQYPGEGLQLLLKATKADDKGSNKGFEATYRKETTSFHLEKGSVQVSDS | 1397 |
| | AVYFCALSHTGSSNTGKLIFGQGTRLQVKP | |
| | (X = any amino acid) | |
| α chain with WT signal | MNYSPGLVSLILLLLGRTRGNSVTQMEGPVTLSEEAFLTINCTYTATGYPSL | 1398 |
| peptide, Cα | FWYVQYPGEGLQLLLKATKADDKGSNKGFEATYRKETTSFHLEKGSVQVSDS | |
| (substituted) | AVYFCALSHTGSSNTGKLIFGQGTRLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAYSPGLVSLILLLLGRTRGNSVTQMEGPVTLSEEAFLTINCTYTATGYPSL | 1399 |
| alternative signal | FWYVQYPGEGLQLLLKATKADDKGSNKGFEATYRKETTSFHLEKGSVQVSDS | |
| peptide, Cα | AVYFCALSHTGSSNTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α | MHYSPGLVSLILLLLGRTRGNSVTQMEGPVTLSEEAFLTINCTYTATGYPSL | 1400 |
| chain with | FWYVQYPGEGLQLLLKATKADDKGSNKGFEATYRKETTSFHLEKGSVQVSDS | |
| alternative signal | AVYFCALSHTGSSNTGKLIFGQGTRLQVKPNIQNPEPAVYQLKDPRSQDSTL | |
| peptide, Cα | CLFTDFDSQINVPKTMESGTFITDKCVLDMKAMDSKSNGAIAWSNQTSFTCQ | |
| (substituted) | DIFKETNATYPSSDVPCDATLTEKSFETDMNLNFQNLLVIVLRILLLKVAGF | |
| | NLLMTLRLWSS | |
| CDR1β | SGHAT | 2391 |
| CDR2β | FQNNGV | 2392 |
| CDR3β | ASSTGGGRHQPQH | 2393 |
| Vβ without signal | GVAQSPRYKIIEKRQSVAFWCNPISGHATLYWYQQILGQGPKLLIQFQNNGV | 2394 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2395 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2396 |
| | WYQQILGQGPKLLIQFQNNGVVDDSQLPKDRFSAERLKGVDSTLKIQPAKLE | 2397 |
| | DSAVYLCASSTGGGRHQPQHFGDGTRLSIL | |
| | (X = any amino acid) | |
| β chain with WT signal | MGTRLLCWAALCLLGAELTEAGVAQSPRYKIIEKRQSVAFWCNPISGHATLY | 2398 |
| peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2399 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2400 |
| signal peptide, Cβ | WYQQILGQGPKLLIQFQNNGVVDDSQLPKDRESAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR040 interacts with and/or is specific for p53. In some embodiments, the peptide is from a neoantigen of p53. In some embodiments, the neoantigen has the amino acid change R248Q relative to the wild type p53 sequence. In some embodiments, TCR040 interacts with the neoantigen in the context of HLA-A*02:01, as described in International Publication No. WO 2019/067243, incorporated herein by reference in its entirety.

TABLE 6AO
Amino acid sequences of TCR041.

| | | SEQ |
|---|---|---|
| | | ID |
| Description | Sequence | NO: |
| CDR1α | ATGYPS | 1401 |
| CDR2α | ATKADDK | 1402 |
| CDR3α | ALSQTGSSKTGKLI | 1403 |
| Vα without signal | NSVTQMEGPVTLSEEAFLTINCTYTATGYPSLFWYVQYPGEGLQLLLKATKA | 1404 |
| peptide (SignalP) | DDKGSNKGFEATYRKETTSFHLEKGSVQVSDSAVYFCALSQTGSSKTGKLIF | |
| | GQGTRLQVKP | |
| Vα without signal | GNSVTQMEGPVTLSEEAFLTINCTYTATGYPSLFWYVQYPGEGLQLLLKATK | 1405 |
| peptide (IMGT) | ADDKGSNKGFEATYRKETTSFHLEKGSVQVSDSAVYFCALSQTGSSKTGKLI | |
| | FGQGTRLQVKP | |
| Vα | MXYSPGLVSLILLLLGRTRGNSVTQMEGPVTLSEEAFLTINCTYTATGYPSL | 1406 |
| | FWYVQYPGEGLQLLLKATKADDKGSNKGFEATYRKETTSFHLEKGSVQVSDS | 1407 |
| | AVYFCALSQTGSSKTGKLIFGQGTRLQVKP | |
| | (X = any amino acid) | |
| α chain with WT signal | MNYSPGLVSLILLLLGRTRGNSVTQMEGPVTLSEEAFLTINCTYTATGYPSL | 1408 |
| peptide, Cα | FWYVQYPGEGLQLLLKATKADDKGSNKGFEATYRKETTSFHLEKGSVQVSDS | |
| (substituted) | AVYFCALSQTGSSKTGKLIFGQGTRLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α | MAYSPGLVSLILLLLGRTRGNSVTQMEGPVTLSEEAFLTINCTYTATGYPSL | 1409 |
| chain with | FWYVQYPGEGLQLLLKATKADDKGSNKGFEATYRKETTSFHLEKGSVQVSDS | |
| alternative signal | AVYFCALSQTGSSKTGKLIFGQGTRLQVKPNIQNPEPAVYQLKDPRSQDSTL | |
| peptide, Cα | CLFTDFDSQINVPKTMESGTFITDKCVLDMKAMDSKSNGAIAWSNQTSFTCQ | |
| (substituted) | DIFKETNATYPSSDVPCDATLTEKSFETDMNLNFQNLLVIVLRILLLKVAGF | |
| | NLLMTLRLWSS | |
| α chain with | MHYSPGLVSLILLLLGRTRGNSVTQMEGPVTLSEEAFLTINCTYTATGYPSL | 1410 |
| alternative signal | FWYVQYPGEGLQLLLKATKADDKGSNKGFEATYRKETTSFHLEKGSVQVSDS | |
| peptide, Cα | AVYFCALSQTGSSKTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | SGHAT | 2401 |
| CDR2β | FQNNGV | 2402 |
| CDR3β | ASSTGGGRHQPQH | 2403 |
| Vβ without signal | GVAQSPRYKIIEKRQSVAFWCNPISGHATLYWYQQILGQGPKLLIQFQNNGV | 2404 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2405 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2406 |
| | WYQQILGQGPKLLIQFQNNGVVDDSQLPKDRFSAERLKGVDSTLKIQPAKLE | 2407 |
| | DSAVYLCASSTGGGRHQPQHFGDGTRLSIL | |
| | (X = any amino acid) | |
| β chain with WT signal | MGTRLLCWAALCLLGAELTEAGVAQSPRYKIIEKRQSVAFWCNPISGHATLY | 2408 |
| peptide, Cβ | WYQQILGQGPKLLIQFQNNGVVDDSQLPKDRESAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2409 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2410 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR041 interacts with and/or is specific for p53. In some embodiments, the peptide is from a neoantigen of p53. In some embodiments, the neoantigen has the amino acid change R248Q relative to the wild type p53 sequence. In some embodiments, TCR041 interacts with the neoantigen in the context of HLA-A*02:01, as described in

TABLE 6AP
Amino acid sequences of TCR042.

| | | SEQ |
|---|---|---|
| | | ID |
| Description | Sequence | NO: |
| CDR1α | ATGYPS | 1411 |
| CDR2α | ATKADDK | 1412 |
| CDR3α | ALSQTGSSNTGKLI | 1413 |
| Vα without signal | NSVTQMEGPVTLSEEAFLTINCTYTATGYPSLFWYVQYPGEGLQLLLKATKA | 1414 |
| peptide (SignalP) | DDKGSNKGFEATYRKETTSFHLEKGSVQVSDSAVYFCALSQTGSSNTGKLIF | |
| | GQGTRLQVKP | |
| Vα without signal | GNSVTQMEGPVTLSEEAFLTINCTYTATGYPSLFWYVQYPGEGLQLLLKATK | 1415 |
| peptide (IMGT) | ADDKGSNKGFEATYRKETTSFHLEKGSVQVSDSAVYFCALSQTGSSNTGKLI | |
| | FGQGTRLQVKP | |
| Vα | MXYSPGLVSLILLLLGRTRGNSVTQMEGPVTLSEEAFLTINCTYTATGYPSL | 1416 |
| | FWYVQYPGEGLQLLLKATKADDKGSNKGFEATYRKETTSFHLEKGSVQVSDS | 1417 |
| | AVYFCALSQTGSSNTGKLIFGQGTRLQVKP | |
| | (X = any amino acid) | |
| α chain with WT signal | MNYSPGLVSLILLLLGRTRGNSVTQMEGPVTLSEEAFLTINCTYTATGYPSL | 1418 |
| peptide, Cα | FWYVQYPGEGLQLLLKATKADDKGSNKGFEATYRKETTSFHLEKGSVQVSDS | |
| (substituted) | AVYFCALSQTGSSNTGKLIFGQGTRLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAYSPGLVSLILLLLGRTRGNSVTQMEGPVTLSEEAFLTINCTYTATGYPSL | 1419 |
| alternative signal | FWYVQYPGEGLQLLLKATKADDKGSNKGFEATYRKETTSFHLEKGSVQVSDS | |
| peptide, Cα | AVYFCALSQTGSSNTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHYSPGLVSLILLLLGRTRGNSVTQMEGPVTLSEEAFLTINCTYTATGYPSL | 1420 |
| alternative signal | FWYVQYPGEGLQLLLKATKADDKGSNKGFEATYRKETTSFHLEKGSVQVSDS | |
| peptide, Cα | AVYFCALSQTGSSNTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | SGHAT | 2411 |
| CDR2β | FQNNGV | 2412 |
| CDR3β | ASSTGGGRHQPQH | 2413 |
| Vβ without signal | GVAQSPRYKIIEKRQSVAFWCNPISGHATLYWYQQILGQGPKLLIQFQNNGV | 2414 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2415 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2416 |
| | WYQQILGQGPKLLIQFQNNGVVDDSQLPKDRFSAERLKGVDSTLKIQPAKLE | 2417 |
| | DSAVYLCASSTGGGRHQPQHFGDGTRLSIL | |
| | (X = any amino acid) | |
| β chain with WT signal | MGTRLLCWAALCLLGAELTEAGVAQSPRYKIIEKRQSVAFWCNPISGHATLY | 2418 |
| peptide, Cβ | WYQQILGQGPKLLIQFQNNGVVDDSQLPKDRESAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2419 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2420 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR042 interacts with and/or is specific for p53. In some embodiments, the peptide is from a neoantigen of p53. In some embodiments, the neoantigen has the amino acid change R248Q relative to the wild type p53 sequence. In some embodiments, TCR042 interacts with the neoantigen in the context of HLA-A*02:01, as described in International Publication No. WO 2019/067243, incorporated herein by reference in its entirety.

TABLE 6AQ
Amino acid sequences of TCR043.

| | | SEQ |
|---|---|---|
| | | ID |
| Description | Sequence | NO: |
| CDR1α | ATGYPS | 1421 |
| CDR2α | ATKADDK | 1422 |
| CDR3α | ALSTTGSSNTGKLI | 1423 |
| Vα without signal | NSVTQMEGPVTLSEEAFLTINCTYTATGYPSLFWYVQYPGEGLQLLLKATKA | 1424 |
| peptide (SignalP) | DDKGSNKGFEATYRKETTSFHLEKGSVQVSDSAVYFCALSTTGSSNTGKLIF | |
| | GQGTTLQVKP | |
| Vα without signal | GNSVTQMEGPVTLSEEAFLTINCTYTATGYPSLFWYVQYPGEGLQLLLKATK | 1425 |
| peptide (IMGT) | ADDKGSNKGFEATYRKETTSFHLEKGSVQVSDSAVYFCALSTTGSSNTGKLI | |
| | FGQGTTLQVKP | |
| Vα | MXYSPGLVSLILLLLGRTRGNSVTQMEGPVTLSEEAFLTINCTYTATGYPSL | 1426 |
| | FWYVQYPGEGLQLLLKATKADDKGSNKGFEATYRKETTSFHLEKGSVQVSDS | 1427 |
| | AVYFCALSTTGSSNTGKLIFGQGTTLQVKP | |
| | (X = any amino acid) | |
| α chain with WT signal | MNYSPGLVSLILLLLGRTRGNSVTQMEGPVTLSEEAFLTINCTYTATGYPSL | 1428 |
| peptide, Cα | FWYVQYPGEGLQLLLKATKADDKGSNKGFEATYRKETTSFHLEKGSVQVSDS | |
| (substituted) | AVYFCALSTTGSSNTGKLIFGQGTTLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGE | |
| | NLLMTLRLWSS | |
| α chain with | MAYSPGLVSLILLLLGRTRGNSVTQMEGPVTLSEEAFLTINCTYTATGYPSL | 1429 |
| alternative signal | FWYVQYPGEGLQLLLKATKADDKGSNKGFEATYRKETTSFHLEKGSVQVSDS | |
| peptide, Cα | AVYFCALSTTGSSNTGKLIFGQGTTLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHYSPGLVSLILLLLGRTRGNSVTQMEGPVTLSEEAFLTINCTYTATGYPSL | 1430 |
| alternative signal | FWYVQYPGEGLQLLLKATKADDKGSNKGFEATYRKETTSFHLEKGSVQVSDS | |
| peptide, Cα | AVYFCALSTTGSSNTGKLIFGQGTTLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGE | |
| | NLLMTLRLWSS | |
| CDR1β | SGHAT | 2421 |
| CDR2β | FQNNGV | 2422 |
| CDR3B | ASSTGGGRHQPQH | 2423 |
| Vβ without signal | GVAQSPRYKIIEKRQSVAFWCNPISGHATLYWYQQILGQGPKLLIQFQNNGV | 2424 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2425 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2426 |
| | WYQQILGQGPKLLIQFQNNGVVDDSQLPKDRFSAERLKGVDSTLKIQPAKLE | |
| | DSAVYLCASSTGGGRHQPQHFGDGTRLSIL | 2427 |
| | (X = any amino acid) | |
| β chain with WT signal | MGTRLLCWAALCLLGAELTEAGVAQSPRYKIIEKRQSVAFWCNPISGHATLY | 2428 |
| peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2429 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2430 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR043 interacts with and/or is specific for p53. In some embodiments, the peptide is from a neoantigen of p53. In some embodiments, the neoantigen has the amino acid change R248Q relative to the wild type p53 sequence. In some embodiments, TCR043 interacts with the neoantigen in the context of HLA-A*02:01, as described in

TABLE 6AR
Amino acid sequences of TCR044.

| | | SEQ |
|---|---|---|
| | | ID |
| Description | Sequence | NO: |
| CDR1α | DRGSQS | 1431 |
| CDR2α | IYSNGD | 1432 |
| CDR3α | AVSWYSTLT | 1433 |
| Vα without signal | QQKEVEQNSGPLSVPEGAIASLNCTYSDRGSQSFFWYRQYSGKSPELIMFIY | 1434 |
| peptide (SignalP) | SNGDKEDGRFTAQLNKASQYVSLLIRDSQPSDSATYLCAVSWYSTLTFGKGT | |
| | MLLVSP | |
| Vα without signal | QKEVEQNSGPLSVPEGAIASLNCTYSDRGSQSFFWYRQYSGKSPELIMFIYS | 1435 |
| peptide (IMGT) | NGDKEDGRFTAQLNKASQYVSLLIRDSQPSDSATYLCAVSWYSTLTFGKGTM | |
| | LLVSP | |
| Vα | MXSLRVLLVILWLQLSWVWSQQKEVEQNSGPLSVPEGAIASLNCTYSDRGSQ | 1436 |
| | SFFWYRQYSGKSPELIMFIYSNGDKEDGRFTAQLNKASQYVSLLIRDSQPSD | 1437 |
| | SATYLCAVSWYSTLTFGKGTMLLVSP | |
| | (X = any amino acid) | |
| α chain with WT signal | MKSLRVLLVILWLQLSWVWSQQKEVEQNSGPLSVPEGAIASLNCTYSDRGSQ | 1438 |
| peptide, Cα | SFFWYRQYSGKSPELIMFIYSNGDKEDGRFTAQLNKASQYVSLLIRDSQPSD | |
| (substituted) | SATYLCAVSWYSTLTFGKGTMLLVSPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGENLLM | |
| | TLRLWSS | |
| α | MASLRVLLVILWLQLSWVWSQQKEVEQNSGPLSVPEGAIASLNCTYSDRGSQ | 1439 |
| chain with | SFFWYRQYSGKSPELIMFIYSNGDKEDGRFTAQLNKASQYVSLLIRDSQPSD | |
| alternative signal | SATYLCAVSWYSTLTFGKGTMLLVSPNIQNPEPAVYQLKDPRSQDSTLCLFT | |
| peptide, Cα | DFDSQINVPKTMESGTFITDKCVLDMKAMDSKSNGAIAWSNQTSFTCQDIFK | |
| (substituted) | ETNATYPSSDVPCDATLTEKSFETDMNLNFQNLLVIVLRILLLKVAGENLLM | |
| | TLRLWSS | |
| α | MHSLRVLLVILWLQLSWVWSQQKEVEQNSGPLSVPEGAIASLNCTYSDRGSQ | 1440 |
| chain with | SFFWYRQYSGKSPELIMFIYSNGDKEDGRFTAQLNKASQYVSLLIRDSQPSD | |
| alternative signal | SATYLCAVSWYSTLTFGKGTMLLVSPNIQNPEPAVYQLKDPRSQDSTLCLFT | |
| peptide, Cα | DFDSQINVPKTMESGTFITDKCVLDMKAMDSKSNGAIAWSNQTSFTCQDIFK | |
| (substituted) | ETNATYPSSDVPCDATLTEKSFETDMNLNFQNLLVIVLRILLLKVAGENLLM | |
| | TLRLWSS | |
| CDR1β | WSHSY | 2431 |
| CDR2β | SAAADI | 2432 |
| CDR3B | ASSGSRTDTQY | 2433 |
| Vβ without signal | GITQSPRYKITETGRQVTLMCHQTWSHSYMFWYRQDLGHGLRLIYYSAAADI | 2434 |
| peptide (SignalP) | TDKGEVPDGYVVSRSKTENFPLTLESATRSQTSVYFCASSGSRTDTQYFGPG | |
| | TRLTVL | |
| Vβ without signal | DAGITQSPRYKITETGRQVTLMCHQTWSHSYMFWYRQDLGHGLRLIYYSAAA | 2435 |
| peptide (IMGT) | DITDKGEVPDGYVVSRSKTENFPLTLESATRSQTSVYFCASSGSRTDTQYFG | |
| | PGTRLTVL | |
| Vβ | MXTRLFFYVALCLLWAGHRDAGITQSPRYKITETGRQVTLMCHQTWSHSYMF | 2436 |
| | WYRQDLGHGLRLIYYSAAADITDKGEVPDGYVVSRSKTENFPLTLESATRSQ | 2437 |
| | TSVYFCASSGSRTDTQYFGPGTRLTVL | |
| | (X = any amino acid) | |
| β chain with WT signal | MGTRLFFYVALCLLWAGHRDAGITQSPRYKITETGRQVTLMCHQTWSHSYMF | 2438 |
| peptide, Cβ | WYRQDLGHGLRLIYYSAAADITDKGEVPDGYVVSRSKTENFPLTLESATRSQ | |
| (substituted) | TSVYFCASSGSRTDTQYFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MATRLFFYVALCLLWAGHRDAGITQSPRYKITETGRQVTLMCHQTWSHSYMF | 2439 |
| signal peptide, Cβ | WYRQDLGHGLRLIYYSAAADITDKGEVPDGYVVSRSKTENFPLTLESATRSQ | |
| (substituted) | TSVYFCASSGSRTDTQYFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MHTRLFFYVALCLLWAGHRDAGITQSPRYKITETGRQVTLMCHQTWSHSYMF | 2440 |
| signal peptide, Cβ | WYRQDLGHGLRLIYYSAAADITDKGEVPDGYVVSRSKTENFPLTLESATRSQ | |
| (substituted) | TSVYFCASSGSRTDTQYFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR044 interacts with and/or is specific for p53. In some embodiments, the peptide is from a neoantigen of p53. In some embodiments, the neoantigen has the amino acid change R248Q relative to the wild type p53 sequence. In some embodiments, TCR044 interacts with the neoantigen in the context of HLA-A*02:01, as described in International Publication No. WO 2019/067243, incorporated herein by reference in its entirety.

TABLE 6AS
Amino acid sequences of TCR045.

| | | SEQ |
|---|---|---|
| | | ID |
| Description | Sequence | NO: |
| CDR1α | SVFSS | 1441 |
| CDR2α | VVTGGEV | 1442 |
| CDR3α | AGEFAGNQFY | 1443 |
| Vα without signal | QLLEQSPQFLSIQEGENLTVYCNSSSVFSSLQWYRQEPGEGPVLLVTVVTGG | 1444 |
| peptide (SignalP) | EVKKLKRLTFQFGDARKDSSLHITAAQPGDTGLYLCAGEFAGNQFYFGTGTS | |
| | LTVIP | |
| Vα without signal | TQLLEQSPQFLSIQEGENLTVYCNSSSVFSSLQWYRQEPGEGPVLLVTVVTG | 1445 |
| peptide (IMGT) | GEVKKLKRLTFQFGDARKDSSLHITAAQPGDTGLYLCAGEFAGNQFYFGTGT | |
| | SLTVIP | |
| Vα | MXLKFSVSILWIQLAWVSTQLLEQSPQFLSIQEGENLTVYCNSSSVESSLQW | 1446 |
| | YRQEPGEGPVLLVTVVTGGEVKKLKRLTFQFGDARKDSSLHITAAQPGDTGL | 1447 |
| | YLCAGEFAGNQFYFGTGTSLTVIP | |
| | (X = any amino acid) | |
| α chain with WT signal | MVLKFSVSILWIQLAWVSTQLLEQSPQFLSIQEGENLTVYCNSSSVFSSLQW | 1448 |
| peptide, Cα | YRQEPGEGPVLLVTVVTGGEVKKLKRLTFQFGDARKDSSLHITAAQPGDTGL | |
| (substituted) | YLCAGEFAGNQFYFGTGTSLTVIPNIQNPEPAVYQLKDPRSQDSTLCLFTDE | |
| | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α chain with | MALKFSVSILWIQLAWVSTQLLEQSPQFLSIQEGENLTVYCNSSSVESSLQW | 1449 |
| alternative signal | YRQEPGEGPVLLVTVVTGGEVKKLKRLTFQFGDARKDSSLHITAAQPGDTGL | |
| peptide, Cα | YLCAGEFAGNQFYFGTGTSLTVIPNIQNPEPAVYQLKDPRSQDSTLCLFTDE | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α | MHLKFSVSILWIQLAWVSTQLLEQSPQFLSIQEGENLTVYCNSSSVFSSLQW | 1450 |
| chain with | YRQEPGEGPVLLVTVVTGGEVKKLKRLTFQFGDARKDSSLHITAAQPGDTGL | |
| alternative signal | YLCAGEFAGNQFYFGTGTSLTVIPNIQNPEPAVYQLKDPRSQDSTLCLFTDF | |
| peptide, Cα | DSQINVPKTMESGTFITDKCVLDMKAMDSKSNGAIAWSNQTSFTCQDIFKET | |
| (substituted) | NATYPSSDVPCDATLTEKSFETDMNLNFQNLLVIVLRILLLKVAGENLLMTL | |
| | RLWSS | |
| CDR1β | MGHRA | 2441 |
| CDR2β | YSYEKL | 2442 |
| CDR3B | ASSQVGLTYEQY | 2443 |
| Vβ without signal | EVTQTPKHLVMGMTNKKSLKCEQHMGHRAMYWYKQKAKKPPELMFVYSYEKL | 2444 |
| peptide (SignalP) | SINESVPSRESPECPNSSLLNLHLHALQPEDSALYLCASSQVGLTYEQYFGP | |
| | GTRLTVT | |
| Vβ without signal | DTEVTQTPKHLVMGMTNKKSLKCEQHMGHRAMYWYKQKAKKPPELMFVYSYE | 2445 |
| peptide (IMGT) | KLSINESVPSRESPECPNSSLLNLHLHALQPEDSALYLCASSQVGLTYEQYF | |
| | GPGTRLTVT | |
| Vβ | MXCRLLCCAVLCLLGAVPIDTEVTQTPKHLVMGMTNKKSLKCEQHMGHRAMY | 2446 |
| | WYKQKAKKPPELMFVYSYEKLSINESVPSRESPECPNSSLLNLHLHALQPED | 2447 |
| | SALYLCASSQVGLTYEQYFGPGTRLTVT | |
| | (X = any amino acid) | |
| β chain with WT signal | MGCRLLCCAVLCLLGAVPIDTEVTQTPKHLVMGMTNKKSLKCEQHMGHRAMY | 2448 |
| peptide, Cβ | WYKQKAKKPPELMFVYSYEKLSINESVPSRESPECPNSSLLNLHLHALQPED | |
| (substituted) | SALYLCASSQVGLTYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MACRLLCCAVLCLLGAVPIDTEVTQTPKHLVMGMTNKKSLKCEQHMGHRAMY | 2449 |
| signal peptide, Cβ | WYKQKAKKPPELMFVYSYEKLSINESVPSRESPECPNSSLLNLHLHALQPED | |
| (substituted) | SALYLCASSQVGLTYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHCRLLCCAVLCLLGAVPIDTEVTQTPKHLVMGMTNKKSLKCEQHMGHRAMY | 2450 |
| signal peptide, Cβ | WYKQKAKKPPELMFVYSYEKLSINESVPSRESPECPNSSLLNLHLHALQPED | |
| (substituted) | SALYLCASSQVGLTYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR045 interacts with and/or is specific for p53. In some embodiments, the peptide is from a neoantigen of p53. In some embodiments, the neoantigen has the amino acid change R248Q relative to the wild type p53 sequence. In some embodiments, TCR045 interacts with the neoantigen in the context of HLA-A*02:01, as described in

TABLE 6AT
Amino acid sequences of TCR046.

| | | SEQ |
|---|---|---|
| | | ID |
| Description | Sequence | NO: |
| CDR1α | VSGNPY | 1451 |
| CDR2α | YITGDNLV | 1452 |
| CDR3α | AVRDNSGGSNYKLT | 1453 |
| Vα without signal | QSVAQPEDQVNVAEGNPLTVKCTYSVSGNPYLFWYVQYPNRGLQFLLKYITG | 1454 |
| peptide (SignalP) | DNLVKGSYGFEAEFNKSQTSFHLKKPSALVSDSALYFCAVRDNSGGSNYKLT | |
| | FGKGTLLTVNP | |
| Vα without signal | AQSVAQPEDQVNVAEGNPLTVKCTYSVSGNPYLFWYVQYPNRGLQFLLKYIT | 1455 |
| peptide (IMGT) | GDNLVKGSYGFEAEFNKSQTSFHLKKPSALVSDSALYFCAVRDNSGGSNYKL | |
| | TFGKGTLLTVNP | |
| Vα | MXSAPISMLAMLFTLSGLRAQSVAQPEDQVNVAEGNPLTVKCTYSVSGNPYL | 1456 |
| | FWYVQYPNRGLQFLLKYITGDNLVKGSYGFEAEFNKSQTSFHLKKPSALVSD | 1457 |
| | SALYFCAVRDNSGGSNYKLTFGKGTLLTVNP | |
| | (X = any amino acid) | |
| α chain with WT signal | MASAPISMLAMLFTLSGLRAQSVAQPEDQVNVAEGNPLTVKCTYSVSGNPYL | 1458 |
| peptide, Cα | FWYVQYPNRGLQFLLKYITGDNLVKGSYGFEAEFNKSQTSFHLKKPSALVSD | |
| | SALYFCAVRDNSGGSNYKLTFGKGTLLTVNPNIQNPEPAVYQLKDPRSQDST | |
| (substituted) | LCLFTDFDSQINVPKTMESGTFITDKCVLDMKAMDSKSNGAIAWSNQTSFTC | 1459 |
| | QDIFKETNATYPSSDVPCDATLTEKSFETDMNLNFQNLLVIVLRILLLKVAG | |
| | FNLLMTLRLWSS | |
| α | MHSAPISMLAMLFTLSGLRAQSVAQPEDQVNVAEGNPLTVKCTYSVSGNPYL | 1460 |
| chain with | FWYVQYPNRGLQFLLKYITGDNLVKGSYGFEAEFNKSQTSFHLKKPSALVSD | |
| alternative signal | SALYFCAVRDNSGGSNYKLTFGKGTLLTVNPNIQNPEPAVYQLKDPRSQDST | |
| peptide, Cα | LCLFTDFDSQINVPKTMESGTFITDKCVLDMKAMDSKSNGAIAWSNQTSFTC | |
| (substituted) | QDIFKETNATYPSSDVPCDATLTEKSFETDMNLNFQNLLVIVLRILLLKVAG | |
| | FNLLMTLRLWSS | |
| CDR1β | SGHAT | 2451 |
| CDR2β | FQNNGV | 2452 |
| CDR3β | ASSLGQGQTQY | 2453 |
| Vβ without signal | GVAQSPRYKIIEKRQSVAFWCNPISGHATLYWYQQILGQGPKLLIQFQNNGV | 2454 |
| peptide (SignalP) | VDDSQLPKDRFSAERLKGVDSTLKIQPAKLEDSAVYLCASSLGQGQTQYFGP | |
| | GTRLLVL | |
| Vβ without signal | EAGVAQSPRYKIIEKRQSVAFWCNPISGHATLYWYQQILGQGPKLLIQFQNN | 2455 |
| peptide (IMGT) | GVVDDSQLPKDRFSAERLKGVDSTLKIQPAKLEDSAVYLCASSLGQGQTQYF | |
| | GPGTRLLVL | |
| Vβ | MXTRLLCWAALCLLGAELTEAGVAQSPRYKIIEKRQSVAFWCNPISGHATLY | 2456 |
| | WYQQILGQGPKLLIQFQNNGVVDDSQLPKDRFSAERLKGVDSTLKIQPAKLE | 2457 |
| | DSAVYLCASSLGQGQTQYFGPGTRLLVL | |
| | (X = any amino acid) | |
| β chain with WT signal | MGTRLLCWAALCLLGAELTEAGVAQSPRYKIIEKRQSVAFWCNPISGHATLY | 2458 |
| peptide, Cβ | WYQQILGQGPKLLIQFQNNGVVDDSQLPKDRFSAERLKGVDSTLKIQPAKLE | |
| (substituted) | DSAVYLCASSLGQGQTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2459 |
| signal peptide, Cβ | WYQQILGQGPKLLIQFQNNGVVDDSQLPKDRFSAERLKGVDSTLKIQPAKLE | |
| (substituted) | DSAVYLCASSLGQGQTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2460 |
| signal peptide, Cβ | WYQQILGQGPKLLIQFQNNGVVDDSQLPKDRESAERLKGVDSTLKIQPAKLE | |
| (substituted) | DSAVYLCASSLGQGQTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR046 interacts with and/or is specific for p53. In some embodiments, the peptide is from a neoantigen of p53. In some embodiments, the neoantigen has the amino acid change R248Q relative to the wild type p53 sequence. In some embodiments, TCR046 interacts with the neoantigen in the context of HLA-A*02:01, as described in International Publication No. WO 2019/067243, incorporated herein by reference in its entirety.

TABLE 6AU
Amino acid sequences of TCR047.

| | | SEQ |
|---|---|---|
| | | ID |
| Description | Sequence | NO: |
| CDR1α | SSVSVY | 1461 |
| CDR2α | YLSGSTLV | 1462 |
| CDR3α | AVRGSSGTYKYI | 1463 |
| Vα without signal | QSVTQLDSQVPVFEEAPVELRCNYSSSVSVYLFWYVQYPNQGLQLLLKYLSG | 1464 |
| peptide (SignalP) | STLVESINGFEAEFNKSQTSFHLRKPSVHISDTAEYFCAVRGSSGTYKYIFG | |
| | TGTRLKVLA | |
| Vα without signal | AQSVTQLDSQVPVFEEAPVELRCNYSSSVSVYLFWYVQYPNQGLQLLLKYLS | 1465 |
| peptide (IMGT) | GSTLVESINGFEAEFNKSQTSFHLRKPSVHISDTAEYFCAVRGSSGTYKYIF | |
| | GTGTRLKVLA | |
| Vα | MXLLLVPAFQVIFTLGGTRAQSVTQLDSQVPVFEEAPVELRCNYSSSVSVYL | 1466 |
| | FWYVQYPNQGLQLLLKYLSGSTLVESINGFEAEFNKSQTSFHLRKPSVHISD | 1467 |
| | TAEYFCAVRGSSGTYKYIFGTGTRLKVLA | |
| | (X = any amino acid) | |
| α chain with WT signal | MLLLLVPAFQVIFTLGGTRAQSVTQLDSQVPVFEEAPVELRCNYSSSVSVYL | 1468 |
| peptide, Cα | FWYVQYPNQGLQLLLKYLSGSTLVESINGFEAEFNKSQTSFHLRKPSVHISD | |
| (substituted) | TAEYFCAVRGSSGTYKYIFGTGTRLKVLANIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α | MALLLVPAFQVIFTLGGTRAQSVTQLDSQVPVFEEAPVELRCNYSSSVSVYL | 1469 |
| chain with | FWYVQYPNQGLQLLLKYLSGSTLVESINGFEAEFNKSQTSFHLRKPSVHISD | |
| alternative signal | TAEYFCAVRGSSGTYKYIFGTGTRLKVLANIQNPEPAVYQLKDPRSQDSTLC | |
| peptide, Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| α chain with | MHLLLVPAFQVIFTLGGTRAQSVTQLDSQVPVFEEAPVELRCNYSSSVSVYL | 1470 |
| alternative signal | FWYVQYPNQGLQLLLKYLSGSTLVESINGFEAEFNKSQTSFHLRKPSVHISD | |
| peptide, Cα | TAEYFCAVRGSSGTYKYIFGTGTRLKVLANIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| CDR1β | MDHEN | 2461 |
| CDR2β | SYDVKM | 2462 |
| CDR3β | ASKGDQNTEAF | 2463 |
| Vβ without signal | SRYLVKRTGEKVFLECVQDMDHENMFWYRQDPGLGLRLIYFSYDVKMKEKGD | 2464 |
| peptide (SignalP) | IPEGYSVSREKKERFSLILESASTNQTSMYLCASKGDQNTEAFFGQGTRLTV | |
| | V | |
| Vβ without signal | DVKVTQSSRYLVKRTGEKVFLECVQDMDHENMFWYRQDPGLGLRLIYFSYDV | 2465 |
| peptide (IMGT) | KMKEKGDIPEGYSVSREKKERFSLILESASTNQTSMYLCASKGDQNTEAFFG | |
| | QGTRLTVV | |
| Vβ | MXIRLLCRVAFCFLAVGLVDVKVTQSSRYLVKRTGEKVFLECVQDMDHENMF | 2466 |
| | WYRQDPGLGLRLIYFSYDVKMKEKGDIPEGYSVSREKKERFSLILESASTNQ | 2467 |
| | TSMYLCASKGDQNTEAFFGQGTRLTVV | |
| | (X = any amino acid) | |
| β chain with WT signal | MGIRLLCRVAFCFLAVGLVDVKVTQSSRYLVKRTGEKVFLECVQDMDHENME | 2468 |
| peptide, Cβ | WYRQDPGLGLRLIYFSYDVKMKEKGDIPEGYSVSREKKERFSLILESASTNQ | |
| (substituted) | TSMYLCASKGDQNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MAIRLLCRVAFCFLAVGLVDVKVTQSSRYLVKRTGEKVFLECVQDMDHENME | 2469 |
| signal peptide, Cβ | WYRQDPGLGLRLIYFSYDVKMKEKGDIPEGYSVSREKKERFSLILESASTNQ | |
| (substituted) | TSMYLCASKGDQNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MHIRLLCRVAFCFLAVGLVDVKVTQSSRYLVKRTGEKVFLECVQDMDHENME | 2470 |
| signal peptide, Cβ | WYRQDPGLGLRLIYFSYDVKMKEKGDIPEGYSVSREKKERFSLILESASTNQ | |
| (substituted) | TSMYLCASKGDQNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHERCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR047 interacts with and/or is specific for p53. In some embodiments, the peptide is from a neoantigen of p53. In some embodiments, the neoantigen has the amino acid change R248Q relative to the wild type p53 sequence. In some embodiments, TCR047 interacts with the neoantigen in the context of HLA-A*02:01, as described in

TABLE 6AV
Amino acid sequences of TCR048.

| | | SEQ |
|---|---|---|
| | | ID |
| Description | Sequence | NO: |
| CDR1α | TSGENG | 1471 |
| CDR2α | NVLDGL | 1472 |
| CDR3α | AVRDLQTGANNLF | 1473 |
| Vα without signal | QNIDQPTEMTATEGAIVQINCTYQTSGENGLFWYQQHAGEAPTFLSYNVLDG | 1474 |
| peptide (SignalP) | LEEKGRFSSFLSRSKGYSYLLLKELQMKDSASYLCAVRDLQTGANNLFFGTG | |
| | TRLTVIP | |
| Vα without signal | GQNIDQPTEMTATEGAIVQINCTYQTSGFNGLFWYQQHAGEAPTFLSYNVLD | 1475 |
| peptide (IMGT) | GLEEKGRFSSFLSRSKGYSYLLLKELQMKDSASYLCAVRDLQTGANNLFFGT | |
| | GTRLTVIP | |
| Vα | MXGVFLLYVSMKMGGTTGQNIDQPTEMTATEGAIVQINCTYQTSGENGLFWY | 1476 |
| | QQHAGEAPTFLSYNVLDGLEEKGRESSFLSRSKGYSYLLLKELQMKDSASYL | 1477 |
| | CAVRDLQTGANNLFFGTGTRLTVIP | |
| | (X = any amino acid) | |
| α chain with WT signal | MWGVFLLYVSMKMGGTTGQNIDQPTEMTATEGAIVQINCTYQTSGENGLEWY | 1478 |
| peptide, Cα | QQHAGEAPTFLSYNVLDGLEEKGRESSFLSRSKGYSYLLLKELQMKDSASYL | |
| (substituted) | CAVRDLQTGANNLFFGTGTRLTVIPNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α | MAGVFLLYVSMKMGGTTGQNIDQPTEMTATEGAIVQINCTYQTSGENGLFWY | 1479 |
| chain with | QQHAGEAPTFLSYNVLDGLEEKGRESSFLSRSKGYSYLLLKELQMKDSASYL | |
| alternative signal | CAVRDLQTGANNLFFGTGTRLTVIPNIQNPEPAVYQLKDPRSQDSTLCLFTD | |
| peptide, Cα | FDSQINVPKTMESGTFITDKCVLDMKAMDSKSNGAIAWSNQTSFTCQDIFKE | |
| (substituted) | TNATYPSSDVPCDATLTEKSFETDMNLNFQNLLVIVLRILLLKVAGENLLMT | |
| | LRLWSS | |
| α | MHGVFLLYVSMKMGGTTGQNIDQPTEMTATEGAIVQINCTYQTSGENGLFWY | 1480 |
| chain with | QQHAGEAPTFLSYNVLDGLEEKGRFSSFLSRSKGYSYLLLKELQMKDSASYL | |
| alternative signal | CAVRDLQTGANNLFFGTGTRLTVIPNIQNPEPAVYQLKDPRSQDSTLCLFTD | |
| peptide, Cα | FDSQINVPKTMESGTFITDKCVLDMKAMDSKSNGAIAWSNQTSFTCQDIFKE | |
| (substituted) | TNATYPSSDVPCDATLTEKSFETDMNLNFQNLLVIVLRILLLKVAGENLLMT | |
| | LRLWSS | |
| CDR1β | MDHEN | 2471 |
| CDR2B | SYDVKM | 2472 |
| CDR3β | ASSLTFGTTEAF | 2473 |
| Vβ without signal | SRYLVKRTGEKVFLECVQDMDHENMFWYRQDPGLGLRLIYFSYDVKMKEKGD | 2474 |
| peptide (SignalP) | IPEGYSVSREKKERFSLILESASTNQTSMYLCASSLTFGTTEAFFGQGTRLT | |
| | VV | |
| Vβ without signal | DVKVTQSSRYLVKRTGEKVFLECVQDMDHENMFWYRQDPGLGLRLIYFSYDV | 2475 |
| peptide (IMGT) | KMKEKGDIPEGYSVSREKKERFSLILESASTNQTSMYLCASSLTFGTTEAFF | |
| | GQGTRLTVV | |
| Vβ | MXIRLLCRVAFCFLAVGLVDVKVTQSSRYLVKRTGEKVFLECVQDMDHENME | 2476 |
| | WYRQDPGLGLRLIYFSYDVKMKEKGDIPEGYSVSREKKERFSLILESASTNQ | 2477 |
| | TSMYLCASSLTFGTTEAFFGQGTRLTVV | |
| | (X = any amino acid) | |
| β chain with WT signal | MGIRLLCRVAFCFLAVGLVDVKVTQSSRYLVKRTGEKVFLECVQDMDHENMF | 2478 |
| peptide, Cβ | WYRQDPGLGLRLIYFSYDVKMKEKGDIPEGYSVSREKKERFSLILESASTNQ | |
| (substituted) | TSMYLCASSLTFGTTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MAIRLLCRVAFCFLAVGLVDVKVTQSSRYLVKRTGEKVFLECVQDMDHENMF | 2479 |
| signal peptide, Cβ | WYRQDPGLGLRLIYFSYDVKMKEKGDIPEGYSVSREKKERFSLILESASTNQ | |
| (substituted) | TSMYLCASSLTFGTTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHIRLLCRVAFCFLAVGLVDVKVTQSSRYLVKRTGEKVFLECVQDMDHENME | 2480 |
| signal peptide, Cβ | WYRQDPGLGLRLIYFSYDVKMKEKGDIPEGYSVSREKKERFSLILESASTNQ | |
| (substituted) | TSMYLCASSLTFGTTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR048 interacts with and/or is specific for p53. In some embodiments, the peptide is from a neoantigen of p53. In some embodiments, the neoantigen has the amino acid change R248Q relative to the wild type p53 sequence. In some embodiments, TCR048 interacts with the neoantigen in the context of HLA-A*02:01, as described in International Publication No. WO 2019/067243, incorporated herein by reference in its entirety.

TABLE 6AW
Amino acid sequences of TCR049.

| | | SEQ |
|---|---|---|
| | | ID |
| Description | Sequence | NO: |
| CDR1α | TSGFNG | 1481 |
| CDR2α | NVLDGL | 1482 |
| CDR3α | AFYYGGSQGNLI | 1483 |
| Vα without signal | QNIDQPTEMTATEGAIVQINCTYQTSGFNGLFWYQQHAGEAPTFLSYNVLDG | 1484 |
| peptide (SignalP) | LEEKGRESSFLSRSKGYSYLLLKELQMKDSASYLCAFYYGGSQGNLIFGKGT | |
| | KLSVKP | |
| Vα without signal | GQNIDQPTEMTATEGAIVQINCTYQTSGFNGLFWYQQHAGEAPTFLSYNVLD | 1485 |
| peptide (IMGT) | GLEEKGRFSSFLSRSKGYSYLLLKELQMKDSASYLCAFYYGGSQGNLIFGKG | |
| | TKLSVKP | |
| Vα | MXGVFLLYVSMKMGGTTGQNIDQPTEMTATEGAIVQINCTYQTSGENGLFWY | 1486 |
| | QQHAGEAPTFLSYNVLDGLEEKGRESSFLSRSKGYSYLLLKELQMKDSASYL | 1487 |
| | CAFYYGGSQGNLIFGKGTKLSVKP | |
| | (X = any amino acid) | |
| α chain with WT signal | MWGVFLLYVSMKMGGTTGQNIDQPTEMTATEGAIVQINCTYQTSGENGLEWY | 1488 |
| peptide, Cα | QQHAGEAPTFLSYNVLDGLEEKGRESSFLSRSKGYSYLLLKELQMKDSASYL | |
| (substituted) | CAFYYGGSQGNLIFGKGTKLSVKPNIQNPEPAVYQLKDPRSQDSTLCLFTDF | |
| | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α chain with | MAGVFLLYVSMKMGGTTGQNIDQPTEMTATEGAIVQINCTYQTSGFNGLFWY | 1489 |
| alternative signal | QQHAGEAPTFLSYNVLDGLEEKGRESSFLSRSKGYSYLLLKELQMKDSASYL | |
| peptide, Cα | CAFYYGGSQGNLIFGKGTKLSVKPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α chain with | MHGVFLLYVSMKMGGTTGQNIDQPTEMTATEGAIVQINCTYQTSGENGLFWY | 1490 |
| alternative signal | QQHAGEAPTFLSYNVLDGLEEKGRESSFLSRSKGYSYLLLKELQMKDSASYL | |
| peptide, Cα | CAFYYGGSQGNLIFGKGTKLSVKPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| CDR1ß | SGHVS | 2481 |
| CDR2ß | FQNEAQ | 2482 |
| CDR3ß | ASSFGSGSTDTQY | 2483 |
| Vß without signal | GVSQSPRYKVAKRGQDVALRCDPISGHVSLFWYQQALGQGPEFLTYFQNEAQ | 2484 |
| peptide (SignalP) | LDKSGLPSDRFFAERPEGSVSTLKIQRTQQEDSAVYLCASSFGSGSTDTQYF | |
| | GPGTRLTVL | |
| Vß without signal | GAGVSQSPRYKVAKRGQDVALRCDPISGHVSLFWYQQALGQGPEFLTYFQNE | 2485 |
| peptide (IMGT) | AQLDKSGLPSDRFFAERPEGSVSTLKIQRTQQEDSAVYLCASSFGSGSTDTQ | |
| | YFGPGTRLTVL | |
| Vß | MXTRLLCWVVLGFLGTDHTGAGVSQSPRYKVAKRGQDVALRCDPISGHVSLF | 2486 |
| | WYQQALGQGPEFLTYFQNEAQLDKSGLPSDRFFAERPEGSVSTLKIQRTQQE | 2487 |
| | DSAVYLCASSFGSGSTDTQYFGPGTRLTVL | |
| | (X = any amino acid) | |
| ß chain with WT signal | MGTRLLCWVVLGFLGTDHTGAGVSQSPRYKVAKRGQDVALRCDPISGHVSLF | 2488 |
| peptide, Cß | WYQQALGQGPEFLTYFQNEAQLDKSGLPSDRFFAERPEGSVSTLKIQRTQQE | |
| (substituted) | DSAVYLCASSFGSGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain with alternative | MATRLLCWVVLGFLGTDHTGAGVSQSPRYKVAKRGQDVALRCDPISGHVSLF | 2489 |
| signal peptide, Cß | WYQQALGQGPEFLTYFQNEAQLDKSGLPSDRFFAERPEGSVSTLKIQRTQQE | |
| (substituted) | DSAVYLCASSFGSGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain with alternative | MHTRLLCWVVLGFLGTDHTGAGVSQSPRYKVAKRGQDVALRCDPISGHVSLF | 2490 |
| signal peptide, Cß | WYQQALGQGPEFLTYFQNEAQLDKSGLPSDRFFAERPEGSVSTLKIQRTQQE | |
| (substituted) | DSAVYLCASSFGSGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR049 interacts with and/or is specific for p53. In some embodiments, the peptide is from a neoantigen of p53. In some embodiments, the neoantigen has the amino acid change R248W relative to the wild type p53 sequence. In some embodiments, TCR049 interacts with the neoantigen in the context of HLA-A*68:01, as described in

TABLE 6AX
Amino acid sequences of TCR050.

| | | SEQ |
|---|---|---|
| | | ID |
| Description | Sequence | NO: |
| CDR1α | VTNFRS | 1491 |
| CDR2α | LTSSGIE | 1492 |
| CDR3α | AGQNYGGSQGNLI | 1493 |
| Vα without signal | EDKVVQSPLSLVVHEGDTVTLNCSYEVTNFRSLLWYKQEKKAPTFLEMLTSS | 1494 |
| peptide (SignalP) | GIEKKSGRLSSILDKKELSSILNITATQTGDSAIYLCAGQNYGGSQGNLIFG | |
| | KGTKLSVKP | |
| Vα without signal | EDKVVQSPLSLVVHEGDTVTLNCSYEVTNERSLLWYKQEKKAPTFLFMLTSS | 1495 |
| peptide (IMGT) | GIEKKSGRLSSILDKKELSSILNITATQTGDSAIYLCAGQNYGGSQGNLIFG | |
| | KGTKLSVKP | |
| Vα | MXKCPQALLAIFWLLLSWVSSEDKVVQSPLSLVVHEGDTVTLNCSYEVTNER | 1496 |
| | SLLWYKQEKKAPTFLFMLTSSGIEKKSGRLSSILDKKELSSILNITATQTGD | 1497 |
| | SAIYLCAGQNYGGSQGNLIFGKGTKLSVKP | |
| | (X = any amino acid) | |
| α chain with WT signal | MMKCPQALLAIFWLLLSWVSSEDKVVQSPLSLVVHEGDTVTLNCSYEVTNER | 1498 |
| peptide, Cα | SLLWYKQEKKAPTFLFMLTSSGIEKKSGRLSSILDKKELSSILNITATQTGD | |
| (substituted) | SAIYLCAGQNYGGSQGNLIFGKGTKLSVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGE | |
| | NLLMTLRLWSS | |
| α chain with | MAKCPQALLAIFWLLLSWVSSEDKVVQSPLSLVVHEGDTVTLNCSYEVTNER | 1499 |
| alternative signal | SLLWYKQEKKAPTFLFMLTSSGIEKKSGRLSSILDKKELSSILNITATQTGD | |
| peptide, Cα | SAIYLCAGQNYGGSQGNLIFGKGTKLSVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHKCPQALLAIFWLLLSWVSSEDKVVQSPLSLVVHEGDTVTLNCSYEVTNER | 1500 |
| alternative signal | SLLWYKQEKKAPTFLFMLTSSGIEKKSGRLSSILDKKELSSILNITATQTGD | |
| peptide, Cα | SAIYLCAGQNYGGSQGNLIFGKGTKLSVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1ß | SNHLY | 2491 |
| CDR2ß | FYNNE | 2492 |
| CDR3ß | ASRDPAYEQY | 2493 |
| Vß without signal | EPEVTQTPSHQVTQMGQEVILRCVPISNHLYFYWYRQILGQKVEFLVSFYNN | 2494 |
| peptide (SignalP) | EISEKSEIFDDQFSVERPDGSNFTLKIRSTKLEDSAMYFCASRDPAYEQYFG | |
| | PGTRLTVT | |
| Vß without signal | EPEVTQTPSHQVTQMGQEVILRCVPISNHLYFYWYRQILGQKVEFLVSFYNN | 2495 |
| peptide (IMGT) | EISEKSEIFDDQFSVERPDGSNFTLKIRSTKLEDSAMYFCASRDPAYEQYFG | |
| | PGTRLTVT | |
| Vß | MXTWLVCWAIFSLLKAGLTEPEVTQTPSHQVTQMGQEVILRCVPISNHLYFY | 2496 |
| | WYRQILGQKVEFLVSFYNNEISEKSEIFDDQFSVERPDGSNFTLKIRSTKLE | 2497 |
| | DSAMYFCASRDPAYEQYFGPGTRLTVT | |
| | (X = any amino acid) | |
| ß chain with WT signal | MDTWLVCWAIFSLLKAGLTEPEVTQTPSHQVTQMGQEVILRCVPISNHLYFY | 2498 |
| peptide, Cß | WYRQILGQKVEFLVSFYNNEISEKSEIFDDQFSVERPDGSNFTLKIRSTKLE | |
| (substituted) | DSAMYFCASRDPAYEQYFGPGTRLTVTEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| ß chain with alternative | MATWLVCWAIFSLLKAGLTEPEVTQTPSHQVTQMGQEVILRCVPISNHLYFY | 2499 |
| signal peptide, Cß | WYRQILGQKVEFLVSFYNNEISEKSEIFDDQFSVERPDGSNFTLKIRSTKLE | |
| (substituted) | DSAMYFCASRDPAYEQYFGPGTRLTVTEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| ß chain with alternative | MHTWLVCWAIFSLLKAGLTEPEVTQTPSHQVTQMGQEVILRCVPISNHLYFY | 2500 |
| signal peptide, Cß | WYRQILGQKVEFLVSFYNNEISEKSEIFDDQFSVERPDGSNFTLKIRSTKLE | |
| (substituted) | DSAMYFCASRDPAYEQYFGPGTRLTVTEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR050 interacts with and/or is specific for p53. In some embodiments, the peptide is from a neoantigen of p53. In some embodiments, the neoantigen has the amino acid change R248W relative to the wild type p53 sequence. In some embodiments, TCR050 interacts with the neoantigen in the context of HLA-A*02:01, as described in International Publication No. WO 2019/067243, incorporated herein by reference in its entirety.

TABLE 6AY
Amino acid sequences of TCR051.

| | | SEQ |
|---|---|---|
| | | ID |
| Description | Sequence | NO: |
| CDR1α | DRGSQS | 1501 |
| CDR2α | IYSNGD | 1502 |
| CDR3α | AVTLCGGYNKLI | 1503 |
| Vα without signal | QQKEVEQNSGPLSVPEGAIASLNCTYSDRGSQSFFWYRQYSGKSPELIMFIY | 1504 |
| peptide (SignalP) | SNGDKEDGRFTAQLNKASQYVSLLIRDSQPSDSATYLCAVTLCGGYNKLIFG | |
| | AGTRLAVHP | |
| Vα without signal | QKEVEQNSGPLSVPEGAIASLNCTYSDRGSQSFFWYRQYSGKSPELIMFIYS | 1505 |
| peptide (IMGT) | NGDKEDGRFTAQLNKASQYVSLLIRDSQPSDSATYLCAVTLCGGYNKLIFGA | |
| | GTRLAVHP | |
| Vα | MXSLRVLLVILWLQLSWVWSQQKEVEQNSGPLSVPEGAIASLNCTYSDRGSQ | 1506 |
| | SFFWYRQYSGKSPELIMFIYSNGDKEDGRFTAQLNKASQYVSLLIRDSQPSD | 1507 |
| | SATYLCAVTLCGGYNKLI FGAGTRLAVHP | |
| | (X = any amino acid) | |
| α chain with WT signal | MKSLRVLLVILWLQLSWVWSQQKEVEQNSGPLSVPEGAIASLNCTYSDRGSQ | 1508 |
| peptide, Cα | SFFWYRQYSGKSPELIMFIYSNGDKEDGRFTAQLNKASQYVSLLIRDSQPSD | |
| (substituted) | SATYLCAVTLCGGYNKLIFGAGTRLAVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MASLRVLLVILWLQLSWVWSQQKEVEQNSGPLSVPEGAIASLNCTYSDRGSQ | 1509 |
| alternative signal | SFFWYRQYSGKSPELIMFIYSNGDKEDGRFTAQLNKASQYVSLLIRDSQPSD | |
| peptide, Ca | SATYLCAVTLCGGYNKLIFGAGTRLAVHPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MHSLRVLLVILWLQLSWVWSQQKEVEQNSGPLSVPEGAIASLNCTYSDRGSQ | 1510 |
| alternative signal | SFFWYRQYSGKSPELIMFIYSNGDKEDGRFTAQLNKASQYVSLLIRDSQPSD | |
| peptide, Ca | SATYLCAVTLCGGYNKLIFGAGTRLAVHPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| CDR1ß | LNHDA | 2501 |
| CDR2ß | SQIVND | 2502 |
| CDR3ß | ASSSRDYEQY | 2503 |
| Vß without signal | GITQSPKYLFRKEGQNVTLSCEQNLNHDAMYWYRQDPGQGLRLIYYSQIVND | 2504 |
| peptide (SignalP) | FQKGDIVEGYSVSREKKESFPLTVTSAQKNPTAFYLCASSSRDYEQYFGPGT | |
| | RLTVT | |
| Vß without signal | DGGITQSPKYLFRKEGQNVTLSCEQNLNHDAMYWYRQDPGQGLRLIYYSQIV | 2505 |
| peptide (IMGT) | NDFQKGDIVEGYSVSREKKESFPLTVTSAQKNPTAFYLCASSSRDYEQYFGP | |
| | GTRLTVI | |
| Vß | MXNQVLCCVVLCFLGANTVDGGITQSPKYLFRKEGQNVTLSCEQNLNHDAMY | 2506 |
| | WYRQDPGQGLRLIYYSQIVNDFQKGDIVEGYSVSREKKESFPLTVTSAQKNP | 2507 |
| | TAFYLCASSSRDYEQYFGPGTRLTVT | |
| | (X = any amino acid) | |
| ß chain with WT signal | MSNQVLCCVVLCFLGANTVDGGITQSPKYLFRKEGQNVTLSCEQNLNHDAMY | 2508 |
| peptide, Cß | WYRQDPGQGLRLIYYSQIVNDFQKGDIVEGYSVSREKKESFPLTVTSAQKNP | |
| (substituted) | TAFYLCASSSRDYEQYFGPGTRLTVTEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain with alternative | MANQVLCCVVLCFLGANTVDGGITQSPKYLFRKEGQNVTLSCEQNLNHDAMY | 2509 |
| signal peptide, Cß | WYRQDPGQGLRLIYYSQIVNDFQKGDIVEGYSVSREKKESFPLTVTSAQKNP | |
| (substituted) | TAFYLCASSSRDYEQYFGPGTRLTVTEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain with alternative | MHNQVLCCVVLCFLGANTVDGGITQSPKYLFRKEGQNVTLSCEQNLNHDAMY | 2510 |
| signal peptide, Cß | WYRQDPGQGLRLIYYSQIVNDFQKGDIVEGYSVSREKKESFPLTVTSAQKNP | |
| (substituted) | TAFYLCASSSRDYEQYFGPGTRLTVTEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR051 interacts with and/or is specific for p53. In some embodiments, the peptide is from a neoantigen of p53. In some embodiments, the neoantigen has the amino acid change R248W relative to the wild type p53 sequence. In some embodiments, TCR051 interacts with the neoantigen in the context of HLA-DPA1*03:01/DPB1*02:01:02, as described in International Publication No. WO 2019/067243, incorporated herein by reference in its entirety.

TABLE 6AZ
Amino acid sequences of TCR052.

| | | SEQ |
|---|---|---|
| | | ID |
| Description | Sequence | NO: |
| CDR1α | SSVSVY | 1511 |
| CDR2α | YLSGSTLV | 1512 |
| CDR3α | AVSDLVRDDKII | 1513 |
| Vα without signal | QSVTQLDSQVPVFEEAPVELRCNYSSSVSVYLFWYVQYPNQGLQLLLKYLSG | 1514 |
| peptide (SignalP) | STLVESINGFEAEFNKSQTSFHLRKPSVHISDTAEYFCAVSDLVRDDKIIFG | |
| | KGTRLHILP | |
| Vα without signal | AQSVTQLDSQVPVFEEAPVELRCNYSSSVSVYLFWYVQYPNQGLQLLLKYLS | 1515 |
| peptide (IMGT) | GSTLVESINGFEAEFNKSQTSFHLRKPSVHISDTAEYFCAVSDLVRDDKIIF | |
| | GKGTRLHILP | |
| Vα | MXLLLVPAFQVIFTLGGTRAQSVTQLDSQVPVFEEAPVELRCNYSSSVSVYL | 1516 |
| | FWYVQYPNQGLQLLLKYLSGSTLVESINGFEAEFNKSQTSFHLRKPSVHISD | 1517 |
| | TAEYFCAVSDLVRDDKIIFGKGTRLHILP | |
| | (X = any amino acid) | |
| α chain with WT signal | MLLLLVPAFQVIFTLGGTRAQSVTQLDSQVPVFEEAPVELRCNYSSSVSVYL | 1518 |
| peptide, Cα | FWYVQYPNQGLQLLLKYLSGSTLVESINGFEAEFNKSQTSFHLRKPSVHISD | |
| (substituted) | TAEYFCAVSDLVRDDKIIFGKGTRLHILPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MALLLVPAFQVIFTLGGTRAQSVTQLDSQVPVFEEAPVELRCNYSSSVSVYL | 1519 |
| alternative signal | FWYVQYPNQGLQLLLKYLSGSTLVESINGFEAEFNKSQTSFHLRKPSVHISD | |
| peptide, Cα | TAEYFCAVSDLVRDDKIIFGKGTRLHILPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MHLLLVPAFQVIFTLGGTRAQSVTQLDSQVPVFEEAPVELRCNYSSSVSVYL | 1520 |
| alternative signal | FWYVQYPNQGLQLLLKYLSGSTLVESINGFEAEFNKSQTSFHLRKPSVHISD | |
| peptide, Cα | TAEYFCAVSDLVRDDKIIFGKGTRLHILPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| CDR1ß | MNHNS | 2511 |
| CDR2ß | SASEGT | 2512 |
| CDR3ß | ASIGGFEAF | 2513 |
| Vß without signal | GVTQTPKFQVLKTGQSMTLQCAQDMNHNSMYWYRQDPGMGLRLIYYSASEGT | 2514 |
| peptide (SignalP) | TDKGEVPNGYNVSRLNKREFSLRLESAAPSQTSVYFCASIGGFEAFFGQGTR | |
| | LTVV | |
| Vß without signal | NAGVTQTPKFQVLKTGQSMTLQCAQDMNHNSMYWYRQDPGMGLRLIYYSASE | 2515 |
| peptide (IMGT) | GTTDKGEVPNGYNVSRLNKREFSLRLESAAPSQTSVYFCASIGGFEAFFGQG | |
| | TRLTVV | |
| Vß | MXIGLLCCVAFSLLWASPVNAGVTQTPKFQVLKTGQSMTLQCAQDMNHNSMY | 2516 |
| | WYRQDPGMGLRLIYYSASEGTTDKGEVPNGYNVSRLNKREFSLRLESAAPSQ | 2517 |
| | TSVYFCASIGGFEAFFGQGTRLTVV | |
| | (X = any amino acid) | |
| ß chain with WT signal | MSIGLLCCVAFSLLWASPVNAGVTQTPKFQVLKTGQSMTLQCAQDMNHNSMY | 2518 |
| peptide, Cß | WYRQDPGMGLRLIYYSASEGTTDKGEVPNGYNVSRLNKREFSLRLESAAPSQ | |
| (substituted) | TSVYFCASIGGFEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| ß chain with alternative | MAIGLLCCVAFSLLWASPVNAGVTQTPKFQVLKTGQSMTLQCAQDMNHNSMY | 2519 |
| signal peptide, Cß | WYRQDPGMGLRLIYYSASEGTTDKGEVPNGYNVSRLNKREFSLRLESAAPSQ | |
| (substituted) | TSVYFCASIGGFEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| ß chain with alternative | MHIGLLCCVAFSLLWASPVNAGVTQTPKFQVLKTGQSMTLQCAQDMNHNSMY | 2520 |
| signal peptide, Cß | WYRQDPGMGLRLIYYSASEGTTDKGEVPNGYNVSRLNKREFSLRLESAAPSQ | |
| (substituted) | TSVYFCASIGGFEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR052 interacts with and/or is specific for p53. In some embodiments, the peptide is from a neoantigen of p53. In some embodiments, the neoantigen has the amino acid change R248W relative to the wild type p53 sequence. In some embodiments, TCR052 interacts with the neoantigen in the context of HLA-A*68:01, as described in International Publication No. WO 2019/067243, incorporated herein by reference in its entirety.

TABLE 6BA
Amino acid sequences of TCR053.

| | | SEQ |
|---|---|---|
| | | ID |
| Description | Sequence | NO: |
| CDR1α | TSGENG | 1521 |
| CDR2α | NVLDGL | 1522 |
| CDR3α | AVYPGGSQGNLI | 1523 |
| Vα without signal | QNIDQPTEMTATEGAIVQINCTYQTSGENGLFWYQQHAGEAPTFLSYNVLDG | 1524 |
| peptide (SignalP) | LEEKGRESSFLSRSKGYSYLLLKELQMKDSASYLCAVYPGGSQGNLIFGKGT | |
| | KLSVKP | |
| Vα without signal | GQNIDQPTEMTATEGAIVQINCTYQTSGENGLFWYQQHAGEAPTFLSYNVLD | 1525 |
| peptide (IMGT) | GLEEKGRESSFLSRSKGYSYLLLKELQMKDSASYLCAVYPGGSQGNLIFGKG | |
| | TKLSVKP | |
| Vα | MXGVFLLYVSMKMGGTTGQNIDQPTEMTATEGAIVQINCTYQTSGENGLFWY | 1526 |
| | QQHAGEAPTFLSYNVLDGLEEKGRESSFLSRSKGYSYLLLKELQMKDSASYL | 1527 |
| | CAVYPGGSQGNLIFGKGTKLSVKP | |
| | (X = any amino acid) | |
| α chain with WT signal | MWGVFLLYVSMKMGGTTGQNIDQPTEMTATEGAIVQINCTYQTSGENGLFWY | 1528 |
| peptide, Cα | QQHAGEAPTFLSYNVLDGLEEKGRESSFLSRSKGYSYLLLKELQMKDSASYL | |
| (substituted) | CAVYPGGSQGNLIFGKGTKLSVKPNIQNPEPAVYQLKDPRSQDSTLCLFTDE | |
| | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α chain with | MAGVFLLYVSMKMGGTTGQNIDQPTEMTATEGAIVQINCTYQTSGENGLEWY | 1529 |
| alternative signal | QQHAGEAPTFLSYNVLDGLEEKGRESSFLSRSKGYSYLLLKELQMKDSASYL | |
| peptide, Cα | CAVYPGGSQGNLIFGKGTKLSVKPNIQNPEPAVYQLKDPRSQDSTLCLFTDE | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α chain with | MHGVFLLYVSMKMGGTTGQNIDQPTEMTATEGAIVQINCTYQTSGENGLFWY | 1530 |
| alternative signal | QQHAGEAPTFLSYNVLDGLEEKGRESSFLSRSKGYSYLLLKELQMKDSASYL | |
| peptide, Cα | CAVYPGGSQGNLIFGKGTKLSVKPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| CDR1ß | SGHAT | 2521 |
| CDR2ß | FQNNGV | 2522 |
| CDR3ß | ASSLGTGSTDTQY | 2523 |
| Vß without signal | GVAQSPRYKIIEKRQSVAFWCNPISGHATLYWYQQILGQGPKLLIQFQNNGV | 2524 |
| peptide (SignalP) | VDDSQLPKDRFSAERLKGVDSTLKIQPAKLEDSAVYLCASSLGTGSTDTQYF | |
| | GPGTRLTVL | |
| Vß without signal | EAGVAQSPRYKIIEKRQSVAFWCNPISGHATLYWYQQILGQGPKLLIQFQNN | 2525 |
| peptide (IMGT) | GVVDDSQLPKDRFSAERLKGVDSTLKIQPAKLEDSAVYLCASSLGTGSTDTQ | |
| | YFGPGTRLTVL | |
| Vß | MXTRLLCWAALCLLGAELTEAGVAQSPRYKIIEKRQSVAFWCNPISGHATLY | 2526 |
| | WYQQILGQGPKLLIQFQNNGVVDDSQLPKDRFSAERLKGVDSTLKIQPAKLE | 2527 |
| | DSAVYLCASSLGTGSTDTQYFGPGTRLTVL | |
| | (X = any amino acid) | |
| ß chain with WT signal | MGTRLLCWAALCLLGAELTEAGVAQSPRYKIIEKRQSVAFWCNPISGHATLY | 2528 |
| peptide, Cß | WYQQILGQGPKLLIQFQNNGVVDDSQLPKDRFSAERLKGVDSTLKIQPAKLE | |
| (substituted) | DSAVYLCASSLGTGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain with alternative | MATRLLCWAALCLLGAELTEAGVAQSPRYKIIEKRQSVAFWCNPISGHATLY | 2529 |
| signal peptide, Cß | WYQQILGQGPKLLIQFQNNGVVDDSQLPKDRFSAERLKGVDSTLKIQPAKLE | |
| (substituted) | DSAVYLCASSLGTGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain with alternative | MHTRLLCWAALCLLGAELTEAGVAQSPRYKIIEKRQSVAFWCNPISGHATLY | 2530 |
| signal peptide, Cß | WYQQILGQGPKLLIQFQNNGVVDDSQLPKDRFSAERLKGVDSTLKIQPAKLE | |
| (substituted) | DSAVYLCASSLGTGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR053 interacts with and/or is specific for p53. In some embodiments, the peptide is from a neoantigen of p53. In some embodiments, the neoantigen has the amino acid change R248W relative to the wild type p53 sequence. In some embodiments, TCR053 interacts with the neoantigen in the context of HLA-A*68:01, as described in

TABLE 6BB
Amino acid sequences of TCR054.

| | | SEQ |
|---|---|---|
| | | ID |
| Description | Sequence | NO: |
| CDR1α | DRGSQS | 1531 |
| CDR2α | IYSNGD | 1532 |
| CDR3α | AVTLSGGYNKLI | 1533 |
| Vα w/o signal peptide | QQKEVEQNSGPLSVPEGAIASLNCTYSDRGSQSFFWYRQYSGKSPELIMFIY | 1534 |
| (SignalP) | SNGDKEDGRFTAQLNKASQYVSLLIRDSQPSDSATYLCAVTLSGGYNKLIFG | |
| | AGTRLAVHP | |
| Vα w/o signal peptide | QKEVEQNSGPLSVPEGAIASLNCTYSDRGSQSFFWYRQYSGKSPELIMFIYS | 1535 |
| (IMGT) | NGDKEDGRFTAQLNKASQYVSLLIRDSQPSDSATYLCAVTLSGGYNKLIFGA | |
| | GTRLAVHP | |
| Vα | MXSLRVLLVILWLQLSWVWSQQKEVEQNSGPLSVPEGAIASLNCTYSDRGSQ | 1536 |
| | SFFWYRQYSGKSPELIMFIYSNGDKEDGRFTAQLNKASQYVSLLIRDSQPSD | 1537 |
| | SATYLCAVTLSGGYNKLIFGAGTRLAVHP | |
| | (X = any amino acid) | |
| α chain w/WT signal | MKSLRVLLVILWLQLSWVWSQQKEVEQNSGPLSVPEGAIASLNCTYSDRGSQ | 1538 |
| peptide, Cα | SFFWYRQYSGKSPELIMFIYSNGDKEDGRFTAQLNKASQYVSLLIRDSQPSD | |
| (substituted) | SATYLCAVTLSGGYNKLIFGAGTRLAVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain w/alternative | MASLRVLLVILWLQLSWVWSQQKEVEQNSGPLSVPEGAIASLNCTYSDRGSQ | 1539 |
| signal peptide, Cα | SFFWYRQYSGKSPELIMFIYSNGDKEDGRFTAQLNKASQYVSLLIRDSQPSD | |
| (substituted) | SATYLCAVTLSGGYNKLIFGAGTRLAVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain w/alternative | MHSLRVLLVILWLQLSWVWSQQKEVEQNSGPLSVPEGAIASLNCTYSDRGSQ | 1540 |
| signal peptide, Cα | SFFWYRQYSGKSPELIMFIYSNGDKEDGRFTAQLNKASQYVSLLIRDSQPSD | |
| (substituted) | SATYLCAVTLSGGYNKLIFGAGTRLAVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| CDR1β | LNHDA | 2531 |
| CDR2β | SQIVND | 2532 |
| CDR3β | ASSSRDYEQY | 2533 |
| Vβ w/o signal peptide | GITQSPKYLFRKEGQNVTLSCEQNLNHDAMYWYRQDPGQGLRLIYYSQIVND | 2534 |
| (SignalP) | FQKGDIVEGYSVSREKKESFPLTVTSAQKNPTAFYLCASSSRDYEQYFGPGT | |
| | RLTVT | |
| Vβ w/o signal peptide | DGGITQSPKYLFRKEGQNVTLSCEQNLNHDAMYWYRQDPGQGLRLIYYSQIV | 2535 |
| (IMGT) | NDFQKGDIVEGYSVSREKKESFPLTVTSAQKNPTAFYLCASSSRDYEQYFGP | |
| | GTRLTVT | |
| Vβ | MXNQVLCCVVLCFLGANTVDGGITQSPKYLFRKEGQNVTLSCEQNINHDAMY | 2536 |
| | WYRQDPGQGLRLIYYSQIVNDFQKGDIVEGYSVSREKKESFPLTVTSAQKNP | 2537 |
| | TAFYLCASSSRDYEQYFGPGTRLTVT | |
| | (X = any amino acid) | |
| β chain w/WT signal | MSNQVLCCVVLCFLGANTVDGGITQSPKYLFRKEGQNVTLSCEQNLNHDAMY | 2538 |
| peptide, Cβ | WYRQDPGQGLRLIYYSQIVNDFQKGDIVEGYSVSREKKESFPLTVTSAQKNP | |
| (substituted) | TAFYLCASSSRDYEQYFGPGTRLTVTEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain w/alternative | MANQVLCCVVLCFLGANTVDGGITQSPKYLFRKEGQNVTLSCEQNLNHDAMY | 2539 |
| signal peptide, Cβ | WYRQDPGQGLRLIYYSQIVNDFQKGDIVEGYSVSREKKESFPLTVTSAQKNP | |
| (substituted) | TAFYLCASSSRDYEQYFGPGTRLTVTEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain w/alternative | MHNQVLCCVVLCFLGANTVDGGITQSPKYLFRKEGQNVTLSCEQNLNHDAMY | 2540 |
| signal peptide, Cβ | WYRQDPGQGLRLIYYSQIVNDFQKGDIVEGYSVSREKKESFPLTVTSAQKNP | |
| (substituted) | TAFYLCASSSRDYEQYFGPGTRLTVTEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR054 interacts with and/or is specific for p53. In some embodiments, the peptide is from a neoantigen of p53. In some embodiments, the neoantigen has the amino acid change R248W relative to the wild type p53 sequence. In some embodiments, TCR054 interacts with the neoantigen in the context of DPA1*01:03/DBP1*02:01 as described in International Publication No. WO 2019/067243, incorporated herein by reference in its entirety.

TABLE 6BC
Amino acid sequences of TCR055.

| | | SEQ |
|---|---|---|
| | | ID |
| Description | Sequence | NO: |
| CDR1α | NSASDY | 1541 |
| CDR2α | IRSNMDK | 1542 |
| CDR3α | AEYIQGAQKLV | 1543 |
| Vα without signal | ESVGLHLPTLSVQEGDNSIINCAYSNSASDYFIWYKQESGKGPQFIIDIRSN | 1544 |
| peptide (SignalP) | MDKRQGQRVTVLLNKTVKHLSLQIAATQPGDSAVYFCAEYIQGAQKLVFGQG | |
| | TRLTINP | |
| Vα without signal | GESVGLHLPTLSVQEGDNSIINCAYSNSASDYFIWYKQESGKGPQFIIDIRS | 1545 |
| peptide (IMGT) | NMDKRQGQRVTVLLNKTVKHLSLQIAATQPGDSAVYFCAEYIQGAQKLVFGQ | |
| | GTRLTINP | |
| Vα | MXGIRALFMYLWLQLDWVSRGESVGLHLPTLSVQEGDNSIINCAYSNSASDY | 1546 |
| | FIWYKQESGKGPQFIIDIRSNMDKRQGQRVTVLLNKTVKHLSLQIAATQPGD | 1547 |
| | SAVYFCAEYIQGAQKLVFGQGTRLTINP | |
| | (X = any amino acid) | |
| α chain with WT signal | MAGIRALFMYLWLQLDWVSRGESVGLHLPTLSVQEGDNSIINCAYSNSASDY | 1548 |
| peptide, Cα | FIWYKQESGKGPQFIIDIRSNMDKRQGQRVTVLLNKTVKHLSLQIAATQPGD | |
| (substituted) | SAVYFCAEYIQGAQKLVFGQGTRLTINPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α | MAGIRALFMYLWLQLDWVSRGESVGLHLPTLSVQEGDNSIINCAYSNSASDY | 1549 |
| chain with | FIWYKQESGKGPQFIIDIRSNMDKRQGQRVTVLLNKTVKHLSLQIAATQPGD | |
| alternative signal | SAVYFCAEYIQGAQKLVFGQGTRLTINPNIQNPEPAVYQLKDPRSQDSTLCL | |
| peptide, Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| α chain with | MHGIRALFMYLWLQLDWVSRGESVGLHLPTLSVQEGDNSIINCAYSNSASDY | 1550 |
| alternative signal | FIWYKQESGKGPQFIIDIRSNMDKRQGQRVTVLLNKTVKHLSLQIAATQPGD | |
| peptide, Cα | SAVYFCAEYIQGAQKLVFGQGTRLTINPNIQNPEPAVYQLKDPRSQDSTLCL | |
| (substituted) | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| CDR1β | LGHNA | 2541 |
| CDR2β | YSLEER | 2542 |
| CDR3β | ASSQEDNEQF | 2543 |
| Vβ without signal | ELVPMETGVTQTPRHLVMGMTNKKSLKCEQHLGHNAMYWYKQSAKKPLELMF | 2544 |
| peptide (SignalP) | VYSLEERVENNSVPSRFSPECPNSSHLFLHLHTLQPEDSALYLCASSQEDNE | |
| | QFFGPGTRLTVL | |
| Vβ without signal | ETGVTQTPRHLVMGMTNKKSLKCEQHLGHNAMYWYKQSAKKPLELMFVYSLE | 2545 |
| peptide (IMGT) | ERVENNSVPSRESPECPNSSHLFLHLHTLQPEDSALYLCASSQEDNEQFFGP | |
| | GTRLTVL | |
| Vβ | MXCRLLCCAVLCLLGAGELVPMETGVTQTPRHLVMGMTNKKSLKCEQHLGHN | 2546 |
| | AMYWYKQSAKKPLELMFVYSLEERVENNSVPSRFSPECPNSSHLFLHLHTLQ | 2547 |
| | PEDSALYLCASSQEDNEQFFGPGTRLTVL | |
| | (X = any amino acid) | |
| β chain with WT signal | MGCRLLCCAVLCLLGAGELVPMETGVTQTPRHLVMGMTNKKSLKCEQHLGHN | 2548 |
| peptide, Cβ | AMYWYKQSAKKPLELMFVYSLEERVENNSVPSRESPECPNSSHLFLHLHTLQ | |
| (substituted) | PEDSALYLCASSQEDNEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain with alternative | MACRLLCCAVLCLLGAGELVPMETGVTQTPRHLVMGMTNKKSLKCEQHLGHN | 2549 |
| signal peptide, Cβ | AMYWYKQSAKKPLELMFVYSLEERVENNSVPSRFSPECPNSSHLFLHLHTLQ | |
| (substituted) | PEDSALYLCASSQEDNEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain with alternative | MHCRLLCCAVLCLLGAGELVPMETGVTQTPRHLVMGMTNKKSLKCEQHLGHN | 2550 |
| signal peptide, Cβ | AMYWYKQSAKKPLELMFVYSLEERVENNSVPSRFSPECPNSSHLFLHLHTLQ | |
| (substituted) | PEDSALYLCASSQEDNEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR055 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR055 interacts with the neoantigen in the context of HLA-C*01:02, as described in International Publication No. WO 2021/163477, incorporated herein by reference in its entirety.

TABLE 6BD
Amino acid sequences of TCR056.

| | | SEQ |
|---|---|---|
| | | ID |
| Description | Sequence | NO: |
| CDR1α | DRGSQS | 1551 |
| CDR2α | IYSNGD | 1552 |
| CDR3α | AVNPPVKTSYDKVI | 1553 |
| Vα without signal | QQKEVEQNSGPLSVPEGAIASLNCTYSDRGSQSFFWYRQYSGKSPELIMFIY | 1554 |
| peptide (SignalP) | SNGDKEDGRFTAQLNKASQYVSLLIRDSQPSDSATYLCAVNPPVKTSYDKVI | |
| | FGPGTSLSVIP | |
| Vα without signal | QKEVEQNSGPLSVPEGAIASLNCTYSDRGSQSFFWYRQYSGKSPELIMFIYS | 1555 |
| peptide (IMGT) | NGDKEDGRFTAQLNKASQYVSLLIRDSQPSDSATYLCAVNPPVKTSYDKVIF | |
| | GPGTSLSVIP | |
| Vα | MXSLRVLLVILWLQLSWVWSQQKEVEQNSGPLSVPEGAIASLNCTYSDRGSQ | 1556 |
| | SFFWYRQYSGKSPELIMFIYSNGDKEDGRFTAQLNKASQYVSLLIRDSQPSD | 1557 |
| | SATYLCAVNPPVKTSYDKVIFGPGTSLSVIP | |
| | (X = any amino acid) | |
| α chain with WT signal | MKSLRVLLVILWLQLSWVWSQQKEVEQNSGPLSVPEGAIASLNCTYSDRGSQ | 1558 |
| peptide, Cα | SFFWYRQYSGKSPELIMFIYSNGDKEDGRFTAQLNKASQYVSLLIRDSQPSD | |
| (substituted) | SATYLCAVNPPVKTSYDKVIFGPGTSLSVIPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain with | MASLRVLLVILWLQLSWVWSQQKEVEQNSGPLSVPEGAIASLNCTYSDRGSQ | 1559 |
| alternative signal | SFFWYRQYSGKSPELIMFIYSNGDKEDGRFTAQLNKASQYVSLLIRDSQPSD | |
| peptide, Cα | SATYLCAVNPPVKTSYDKVIFGPGTSLSVIPNIQNPEPAVYQLKDPRSQDST | |
| (substituted) | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α | MHSLRVLLVILWLQLSWVWSQQKEVEQNSGPLSVPEGAIASLNCTYSDRGSQ | 1560 |
| chain with | SFFWYRQYSGKSPELIMFIYSNGDKEDGRFTAQLNKASQYVSLLIRDSQPSD | |
| alternative signal | SATYLCAVNPPVKTSYDKVIFGPGTSLSVIPNIQNPEPAVYQLKDPRSQDST | |
| peptide, Cα | LCLFTDFDSQINVPKTMESGTFITDKCVLDMKAMDSKSNGAIAWSNQTSFTC | |
| (substituted) | QDIFKETNATYPSSDVPCDATLTEKSFETDMNLNFQNLLVIVLRILLLKVAG | |
| | FNLLMTLRLWSS | |
| CDR1β | SGHVS | 2551 |
| CDR2β | FNYEA | 2552 |
| CDR3β | ASSHREPHTGELF | 2553 |
| Vβ without signal | GVSQSPRYKVTKRGQDVALRCDPISGHVSLYWYRQALGQGPEFLTYFNYEAQ | 2554 |
| peptide (SignalP) | QDKSGLPNDRFSAERPEGSISTLTIQRTEQRDSAMYRCASSHREPHTGELFF | |
| | GEGSRLTVL | |
| Vβ without signal | GAGVSQSPRYKVTKRGQDVALRCDPISGHVSLYWYRQALGQGPEFLTYENYE | 2555 |
| peptide (IMGT) | AQQDKSGLPNDRFSAERPEGSISTLTIQRTEQRDSAMYRCASSHREPHTGEL | |
| | FFGEGSRLTVL | |
| Vβ | MXTSLLCWVVLGFLGTDHTGAGVSQSPRYKVTKRGQDVALRCDPISGHVSLY | 2556 |
| | WYRQALGQGPEFLTYFNYEAQQDKSGLPNDRFSAERPEGSISTLTIQRTEQR | 2557 |
| | DSAMYRCASSHREPHTGELFFGEGSRLTVL | |
| | (X = any amino acid) | |
| β chain with WT signal | MGTSLLCWVVLGFLGTDHTGAGVSQSPRYKVTKRGQDVALRCDPISGHVSLY | 2558 |
| peptide, Cβ | WYRQALGQGPEFLTYFNYEAQQDKSGLPNDRESAERPEGSISTLTIQRTEQR | |
| (substituted) | DSAMYRCASSHREPHTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATSLLCWVVLGFLGTDHTGAGVSQSPRYKVTKRGQDVALRCDPISGHVSLY | 2559 |
| signal peptide, Cβ | WYRQALGQGPEFLTYFNYEAQQDKSGLPNDRFSAERPEGSISTLTIQRTEQR | |
| (substituted) | DSAMYRCASSHREPHTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTSLLCWVVLGFLGTDHTGAGVSQSPRYKVTKRGQDVALRCDPISGHVSLY | 2560 |
| signal peptide, Cβ | WYRQALGQGPEFLTYFNYEAQQDKSGLPNDRESAERPEGSISTLTIQRTEQR | |
| (substituted) | DSAMYRCASSHREPHTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR056 interacts with and/or is specific for p53. In some embodiments, the peptide is from a neoantigen of p53. In some embodiments, the neoantigen has the amino acid change R248W relative to the wild type p53 sequence. In some embodiments, TCR056 interacts with the neoantigen in the context of HLA-A*02:01, as described in

TABLE 6BE
Amino acid sequences of TCR057.

| | | SEQ |
|---|---|---|
| | | ID |
| Description | Sequence | NO: |
| CDR1α | TSGENG | 1561 |
| CDR2α | NVLDGL | 1562 |
| CDR3α | AVYTGGFKTI | 1563 |
| Vα without signal | QNIDQPTEMTATEGAIVQINCTYQTSGENGLFWYQQHAGEAPTFLSYNVLDG | 1564 |
| peptide (SignalP) | LEEKGRESSFLSRSKGYSYLLLKELQMKDSASYLCAVYTGGFKTIFGAGTRL | |
| | FVKA | |
| Vα without signal | GQNIDQPTEMTATEGAIVQINCTYQTSGFNGLFWYQQHAGEAPTFLSYNVLD | 1565 |
| peptide (IMGT) | GLEEKGRFSSFLSRSKGYSYLLLKELQMKDSASYLCAVYTGGFKTIFGAGTR | |
| | LFVKA | |
| Vα | MXGVFLLYVSMKMGGTTGQNIDQPTEMTATEGAIVQINCTYQTSGENGLFWY | 1566 |
| | QQHAGEAPTFLSYNVLDGLEEKGRESSFLSRSKGYSYLLLKELQMKDSASYL | 1567 |
| | CAVYTGGFKTIFGAGTRLFVKA | |
| | (X = any amino acid) | |
| α chain with WT signal | MWGVFLLYVSMKMGGTTGQNIDQPTEMTATEGAIVQINCTYQTSGENGLFWY | 1568 |
| peptide, Cα | QQHAGEAPTFLSYNVLDGLEEKGRESSFLSRSKGYSYLLLKELQMKDSASYL | |
| (substituted) | CAVYTGGFKTIFGAGTRLFVKANIQNPEPAVYQLKDPRSQDSTLCLFTDFDS | |
| | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGENLLMTLRL | |
| | WSS | |
| α | MAGVFLLYVSMKMGGTTGQNIDQPTEMTATEGAIVQINCTYQTSGENGLFWY | 1569 |
| chain with | QQHAGEAPTFLSYNVLDGLEEKGRESSFLSRSKGYSYLLLKELQMKDSASYL | |
| alternative signal | CAVYTGGFKTIFGAGTRLFVKANIQNPEPAVYQLKDPRSQDSTLCLFTDFDS | |
| peptide, Cα | QINVPKTMESGTFITDKCVLDMKAMDSKSNGAIAWSNQTSFTCQDIFKETNA | |
| (substituted) | TYPSSDVPCDATLTEKSFETDMNLNFQNLLVIVLRILLLKVAGENLLMTLRL | |
| | WSS | |
| α chain with | MHGVFLLYVSMKMGGTTGQNIDQPTEMTATEGAIVQINCTYQTSGENGLFWY | 1570 |
| alternative signal | QQHAGEAPTFLSYNVLDGLEEKGRESSFLSRSKGYSYLLLKELQMKDSASYL | |
| peptide, Cα | CAVYTGGFKTIFGAGTRLFVKANIQNPEPAVYQLKDPRSQDSTLCLFTDEDS | |
| (substituted) | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGENLLMTLRL | |
| | WSS | |
| CDR1β | SGHAT | 2561 |
| CDR2β | FQNNGV | 2562 |
| CDR3β | ASNLGGGSTDTQY | 2563 |
| Vβ without signal | GVAQSPRYKIIEKRQSVAFWCNPISGHATLYWYQQILGQGPKLLIQFQNNGV | 2564 |
| peptide (SignalP) | VDDSQLPKDRFSAERLKGVDSTLKIQPAKLEDSAVYLCASNLGGGSTDTQYF | |
| | GPGTRLTVL | |
| Vβ without signal | EAGVAQSPRYKIIEKRQSVAFWCNPISGHATLYWYQQILGQGPKLLIQFQNN | 2565 |
| peptide (IMGT) | GVVDDSQLPKDRFSAERLKGVDSTLKIQPAKLEDSAVYLCASNLGGGSTDTQ | |
| | YFGPGTRLTVL | |
| Vβ | MXTRLLCWAALCLLGAELTEAGVAQSPRYKIIEKRQSVAFWCNPISGHATLY | 2566 |
| | WYQQILGQGPKLLIQFQNNGVVDDSQLPKDRFSAERLKGVDSTLKIQPAKLE | 2567 |
| | DSAVYLCASNLGGGSTDTQYFGPGTRLTVL | |
| | (X = any amino acid) | |
| β chain with WT signal | MGTRLLCWAALCLLGAELTEAGVAQSPRYKIIEKRQSVAFWCNPISGHATLY | 2568 |
| peptide, Cβ | WYQQILGQGPKLLIQFQNNGVVDDSQLPKDRFSAERLKGVDSTLKIQPAKLE | |
| (substituted) | DSAVYLCASNLGGGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2569 |
| signal peptide, Cβ | WYQQILGQGPKLLIQFQNNGVVDDSQLPKDRFSAERLKGVDSTLKIQPAKLE | |
| (substituted) | DSAVYLCASNLGGGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2570 |
| signal peptide, Cβ | WYQQILGQGPKLLIQFQNNGVVDDSQLPKDRFSAERLKGVDSTLKIQPAKLE | |
| (substituted) | DSAVYLCASNLGGGSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR057 interacts with and/or is specific for p53. In some embodiments, the peptide is from a neoantigen of p53. In some embodiments, the neoantigen has the amino acid change R248W relative to the wild type p53 sequence. In some embodiments, TCR057 interacts with the neoantigen in the context of HLA-A*68:01, as described in

TABLE 6BF
Amino acid sequences of TCR058.

| | | SEQ |
|---|---|---|
| | | ID |
| Description | Sequence | NO: |
| CDR1α | NSASQS | 1571 |
| CDR2α | VYSSGN | 1572 |
| CDR3α | AVNPKYTGGFKTI | 1573 |
| Vα without signal | QRKEVEQDPGPFNVPEGATVAFNCTYSNSASQSFFWYRQDCRKEPKLLMSVY | 1574 |
| peptide (SignalP) | SSGNEDGRFTAHLNRASQYISLLIRDSKLSDSATYLCAVNPKYTGGFKTIFG | |
| | AGTRLFVKA | |
| Vα without signal | RKEVEQDPGPFNVPEGATVAFNCTYSNSASQSFFWYRQDCRKEPKLLMSVYS | 1575 |
| peptide (IMGT) | SGNEDGRFTAHLNRASQYISLLIRDSKLSDSATYLCAVNPKYTGGFKTIFGA | |
| | GTRLFVKA | |
| Vα | MXSLRVLLVILWLQLSWVWSQRKEVEQDPGPFNVPEGATVAFNCTYSNSASQ | 1576 |
| | SFFWYRQDCRKEPKLLMSVYSSGNEDGRFTAHLNRASQYISLLIRDSKLSDS | 1577 |
| | ATYLCAVNPKYTGGFKTIFGAGTRLFVKA | |
| | (X = any amino acid) | |
| α chain with WT signal | MISLRVLLVILWLQLSWVWSQRKEVEQDPGPFNVPEGATVAFNCTYSNSASQ | 1578 |
| peptide, Cα | SFFWYRQDCRKEPKLLMSVYSSGNEDGRFTAHLNRASQYISLLIRDSKLSDS | |
| (substituted) | ATYLCAVNPKYTGGFKTIFGAGTRLFVKANIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MASLRVLLVILWLQLSWVWSQRKEVEQDPGPFNVPEGATVAFNCTYSNSASQ | 1579 |
| alternative signal | SFFWYRQDCRKEPKLLMSVYSSGNEDGRFTAHLNRASQYISLLIRDSKLSDS | |
| peptide, Cα | ATYLCAVNPKYTGGFKTIFGAGTRLFVKANIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α | MHSLRVLLVILWLQLSWVWSQRKEVEQDPGPFNVPEGATVAFNCTYSNSASQ | 1580 |
| chain with | SFFWYRQDCRKEPKLLMSVYSSGNEDGRFTAHLNRASQYISLLIRDSKLSDS | |
| alternative signal | ATYLCAVNPKYTGGFKTIFGAGTRLFVKANIQNPEPAVYQLKDPRSQDSTLC | |
| peptide, Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| CDR1β | LGHNA | 2571 |
| CDR2β | YSLEER | 2572 |
| CDR3β | ASSQDVTSEWVDTIY | 2573 |
| Vβ without signal | ELVPMETGVTQTPRHLVMGMTNKKSLKCEQHLGHNAMYWYKQSAKKPLELMF | 2574 |
| peptide (SignalP) | VYSLEERVENNSVPSRFSPECPNSSHLFLHLHTLQPEDSALYLCASSQDVTS | |
| | EWVDTIYFGEGSWLTVV | |
| Vβ without signal | ETGVTQTPRHLVMGMTNKKSLKCEQHLGHNAMYWYKQSAKKPLELMFVYSLE | 2575 |
| peptide (IMGT) | ERVENNSVPSRESPECPNSSHLFLHLHTLQPEDSALYLCASSQDVTSEWVDT | |
| | IYFGEGSWLTVV | |
| Vβ | MXCRLLCCAVLCLLGAGELVPMETGVTQTPRHLVMGMTNKKSLKCEQHLGHN | 2576 |
| | AMYWYKQSAKKPLELMFVYSLEERVENNSVPSRFSPECPNSSHLFLHLHTLQ | 2577 |
| | PEDSALYLCASSQDVTSEWVDTIYFGEGSWLTVV | |
| | (X = any amino acid) | |
| β chain with WT signal | MGCRLLCCAVLCLLGAGELVPMETGVTQTPRHLVMGMTNKKSLKCEQHLGHN | 2578 |
| peptide, Cβ | AMYWYKQSAKKPLELMFVYSLEERVENNSVPSRESPECPNSSHLFLHLHTLQ | |
| (substituted) | PEDSALYLCASSQDVTSEWVDTIYFGEGSWLTVVEDLRNVTPPKVSLFEPSK | |
| | AEIANKQKATLVCLARGFFPDHVELSWWVNGKEVHSGVCTDPQAYKESNYSY | |
| | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |
| β chain with alternative | MACRLLCCAVLCLLGAGELVPMETGVTQTPRHLVMGMTNKKSLKCEQHLGHN | 2579 |
| signal peptide, Cβ | AMYWYKQSAKKPLELMFVYSLEERVENNSVPSRESPECPNSSHLFLHLHTLQ | |
| (substituted) | PEDSALYLCASSQDVTSEWVDTIYFGEGSWLTVVEDLRNVTPPKVSLFEPSK | |
| | AEIANKQKATLVCLARGFFPDHVELSWWVNGKEVHSGVCTDPQAYKESNYSY | |
| | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |
| β chain with alternative | MHCRLLCCAVLCLLGAGELVPMETGVTQTPRHLVMGMTNKKSLKCEQHLGHN | 2580 |
| signal peptide, Cβ | AMYWYKQSAKKPLELMFVYSLEERVENNSVPSRFSPECPNSSHLFLHLHTLQ | |
| (substituted) | PEDSALYLCASSQDVTSEWVDTIYFGEGSWLTVVEDLRNVTPPKVSLFEPSK | |
| | AEIANKQKATLVCLARGFFPDHVELSWWVNGKEVHSGVCTDPQAYKESNYSY | |
| | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |

In some embodiments, TCR058 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR058 interacts with the neoantigen in the context of HLA-C*01:02, as described in International Publication No. WO 2021/163477, incorporated herein by reference in its entirety.

TABLE 6BG
Amino acid sequences of TCR059.

| | | SEQ |
|---|---|---|
| | | ID |
| Description | Sequence | NO: |
| CDR1α | NSASQS | 1581 |
| CDR2α | VYSSGN | 1582 |
| CDR3α | VVDDQTGANNLF | 1583 |
| Vα without signal | QRKEVEQDPGPFNVPEGATVAFNCTYSNSASQSFFWYRQDCRKEPKLLMSVY | 1584 |
| peptide (SignalP) | SSGNEDGRFTAQLNRASQYISLLIRDSKLSDSATYLCVVDDQTGANNLFFGT | |
| | GTRLTVIP | |
| Vα without signal | RKEVEQDPGPFNVPEGATVAFNCTYSNSASQSFFWYRQDCRKEPKLLMSVYS | 1585 |
| peptide (IMGT) | SGNEDGRFTAQLNRASQYISLLIRDSKLSDSATYLCVVDDQTGANNLFFGTG | |
| | TRLTVIP | |
| Vα | MXSLRVLLVILWLQLSWVWSQRKEVEQDPGPENVPEGATVAFNCTYSNSASQ | 1586 |
| | SFFWYRQDCRKEPKLLMSVYSSGNEDGRFTAQLNRASQYISLLIRDSKLSDS | 1587 |
| | ATYLCVVDDQTGANNLFFGTGTRLTVIP | |
| | (X = any amino acid) | |
| α chain with WT signal | MISLRVLLVILWLQLSWVWSQRKEVEQDPGPFNVPEGATVAFNCTYSNSASQ | 1588 |
| peptide, Cα | SFFWYRQDCRKEPKLLMSVYSSGNEDGRFTAQLNRASQYISLLIRDSKLSDS | |
| (substituted) | ATYLCVVDDQTGANNLFFGTGTRLTVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α | MASLRVLLVILWLQLSWVWSQRKEVEQDPGPFNVPEGATVAFNCTYSNSASQ | 1589 |
| chain with | SFFWYRQDCRKEPKLLMSVYSSGNEDGRFTAQLNRASQYISLLIRDSKLSDS | |
| alternative signal | ATYLCVVDDQTGANNLFFGTGTRLTVIPNIQNPEPAVYQLKDPRSQDSTLCL | |
| peptide, Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| β | MHSLRVLLVILWLQLSWVWSQRKEVEQDPGPFNVPEGATVAFNCTYSNSASQ | 1590 |
| chain with | SFFWYRQDCRKEPKLLMSVYSSGNEDGRFTAQLNRASQYISLLIRDSKLSDS | |
| alternative signal | ATYLCVVDDQTGANNLFFGTGTRLTVIPNIQNPEPAVYQLKDPRSQDSTLCL | |
| peptide, Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| CDR1β | MNHEY | 2581 |
| CDR2β | SVGAGI | 2582 |
| CDR3β | ASRNLGDTQY | 2583 |
| Vβ without signal | GVTQTPKFQVLKTGQSMTLQCAQDMNHEYMSWYRQDPGMGLRLIHYSVGAGI | 2584 |
| peptide (SignalP) | TDQGEVPNGYNVSRSTTEDFPLRLLSAAPSQTSVYFCASRNLGDTQYFGPGT | |
| | RLTVL | |
| Vβ without signal | NAGVTQTPKFQVLKTGQSMTLQCAQDMNHEYMSWYRQDPGMGLRLIHYSVGA | 2585 |
| peptide (IMGT) | GITDQGEVPNGYNVSRSTTEDFPLRLLSAAPSQTSVYFCASRNLGDTQYFGP | |
| | GTRLTVL | |
| Vβ | MXIGLLCCAALSLLWAGPVNAGVTQTPKFQVLKTGQSMTLQCAQDMNHEYMS | 2586 |
| | WYRQDPGMGLRLIHYSVGAGITDQGEVPNGYNVSRSTTEDEPLRLLSAAPSQ | 2587 |
| | TSVYFCASRNLGDTQYFGPGTRLTVL | |
| | (X = any amino acid) | |
| β chain with WT signal | MSIGLLCCAALSLLWAGPVNAGVTQTPKFQVLKTGQSMTLQCAQDMNHEYMS | 2588 |
| peptide, Cβ | WYRQDPGMGLRLIHYSVGAGITDQGEVPNGYNVSRSTTEDFPLRLLSAAPSQ | |
| (substituted) | TSVYFCASRNLGDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with alternative | MAIGLLCCAALSLLWAGPVNAGVTQTPKFQVLKTGQSMTLQCAQDMNHEYMS | 2589 |
| signal peptide, Cβ | WYRQDPGMGLRLIHYSVGAGITDQGEVPNGYNVSRSTTEDFPLRLLSAAPSQ | |
| (substituted) | TSVYFCASRNLGDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with alternative | MHIGLLCCAALSLLWAGPVNAGVTQTPKFQVLKTGQSMTLQCAQDMNHEYMS | 2590 |
| signal peptide, Cβ | WYRQDPGMGLRLIHYSVGAGITDQGEVPNGYNVSRSTTEDFPLRLLSAAPSQ | |
| (substituted) | TSVYFCASRNLGDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR059 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR059 interacts with the neoantigen in the context of HLA-C*01:02, as described in International Publication No. WO 2021/163477, incorporated herein by reference in its entirety.

TABLE 6BH
Amino acid sequences of TCR060.

| | | SEQ |
|---|---|---|
| | | ID |
| Description | Sequence | NO: |
| CDR1α | TSINN | 1591 |
| CDR2α | IRSNERE | 1592 |
| CDR3α | ATDGETSGSRLT | 1593 |
| Vα without signal | QQGEEDPQALSIQEGENATMNCSYKTSINNLQWYRQNSGRGLVHLILIRSNE | 1594 |
| peptide (SignalP) | REKHSGRLRVTLDTSKKSSSLLITASRAADTASYFCATDGETSGSRLTFGEG | |
| | TQLTVNP | |
| Vα without signal | SQQGEEDPQALSIQEGENATMNCSYKTSINNLQWYRQNSGRGLVHLILIRSN | 1595 |
| peptide (IMGT) | EREKHSGRLRVTLDTSKKSSSLLITASRAADTASYFCATDGETSGSRLTFGE | |
| | GTQLTVNP | |
| Vα | MXTLLGVSLVILWLQLAVNSQQGEEDPQALSIQEGENATMNCSYKTSINNLQ | 1596 |
| | WYRQNSGRGLVHLILIRSNEREKHSGRLRVTLDTSKKSSSLLITASRAADTA | 1597 |
| | SYFCATDGETSGSRLTFGEGTQLTVNP | |
| | (X = any amino acid) | |
| α chain with WT signal | METLLGVSLVILWLQLAVNSQQGEEDPQALSIQEGENATMNCSYKTSINNLQ | 1598 |
| peptide, Cα | WYRQNSGRGLVHLILIRSNEREKHSGRLRVTLDTSKKSSSLLITASRAADTA | |
| (substituted) | SYFCATDGETSGSRLTFGEGTQLTVNPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain | MATLLGVSLVILWLQLAVNSQQGEEDPQALSIQEGENATMNCSYKTSINNLQ | 1599 |
| with | WYRQNSGRGLVHLILIRSNEREKHSGRLRVTLDTSKKSSSLLITASRAADTA | |
| alternative signal | SYFCATDGETSGSRLTFGEGTQLTVNPNIQNPEPAVYQLKDPRSQDSTLCLF | |
| peptide, Cα | TDFDSQINVPKTMESGTFITDKCVLDMKAMDSKSNGAIAWSNQTSFTCQDIF | |
| (substituted) | KETNATYPSSDVPCDATLTEKSFETDMNLNFQNLLVIVLRILLLKVAGENLL | |
| | MTLRLWSS | |
| α | MHTLLGVSLVILWLQLAVNSQQGEEDPQALSIQEGENATMNCSYKTSINNLQ | 1600 |
| chain with | WYRQNSGRGLVHLILIRSNEREKHSGRLRVTLDTSKKSSSLLITASRAADTA | |
| alternative signal | SYFCATDGETSGSRLTFGEGTQLTVNPNIQNPEPAVYQLKDPRSQDSTLCLF | |
| peptide, Cα | TDFDSQINVPKTMESGTFITDKCVLDMKAMDSKSNGAIAWSNQTSFTCQDIF | |
| (substituted) | KETNATYPSSDVPCDATLTEKSFETDMNLNFQNLLVIVLRILLLKVAGENLL | |
| | MTLRLWSS | |
| CDR1β | DFQATT | 2591 |
| CDR2β | SNEGSKA | 2592 |
| CDR3β | SASRGATGQPQH | 2593 |
| Vβ without signal | GSGLGAVVSQHPSWVICKSGTSVKIECRSLDFQATTMFWYRQFPKQSLMLMA | 2594 |
| peptide (SignalP) | TSNEGSKATYEQGVEKDKFLINHASLTLSTLTVTSAHPEDSSFYICSASRGA | |
| | TGQPQHFGDGTRLSIL | |
| β without signal | GAVVSQHPSWVICKSGTSVKIECRSLDFQATTMFWYRQFPKQSLMLMATSNE | 2595 |
| peptide (IMGT) | GSKATYEQGVEKDKFLINHASLTLSTLTVTSAHPEDSSFYICSASRGATGQP | |
| | QHFGDGTRLSIL | |
| Vβ | MXLLLLLLGPGISLLLPGSLAGSGLGAVVSQHPSWVICKSGTSVKIECRSLD | 2596 |
| | FQATTMFWYRQFPKQSLMLMATSNEGSKATYEQGVEKDKFLINHASLTLSTL | 2597 |
| | TVTSAHPEDSSFYICSASRGATGQPQHFGDGTRLSIL | |
| | (X = any amino acid) | |
| β chain with WT signal | MLLLLLLLGPGISLLLPGSLAGSGLGAVVSQHPSWVICKSGTSVKIECRSLD | 2598 |
| peptide, Cβ | FQATTMFWYRQFPKQSLMLMATSNEGSKATYEQGVEKDKFLINHASLTLSTL | |
| (substituted) | TVTSAHPEDSSFYICSASRGATGQPQHFGDGTRLSILEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHERCQVQFHGLSEEDKWPEGSPKPVTQNISA | |
| | EAWGRADCGITSASYQQGVLSATILYEILLGKATLYAVLVSTLVVMAMVKRK | |
| | NS | |
| β chain with alternative | MALLLLLLGPGISLLLPGSLAGSGLGAVVSQHPSWVICKSGTSVKIECRSLD | 2599 |
| signal peptide, Cβ | FQATTMFWYRQFPKQSLMLMATSNEGSKATYEQGVEKDKFLINHASLTLSTL | |
| (substituted) | TVTSAHPEDSSFYICSASRGATGQPQHFGDGTRLSILEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| β chain with alternative | MHLLLLLLGPGISLLLPGSLAGSGLGAVVSQHPSWVICKSGTSVKIECRSLD | 2600 |
| signal peptide, Cβ | FQATTMFWYRQFPKQSLMLMATSNEGSKATYEQGVEKDKFLINHASLTLSTL | |
| (substituted) | TVTSAHPEDSSFYICSASRGATGQPQHFGDGTRLSILEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |

In some embodiments, TCR060 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR060 interacts with the neoantigen in the context of an HLA-DPA1*01:03 chain and an HLA-DPB1*03:01 chain, as described in International Publication No. WO 2021/173902, incorporated herein by reference in its entirety.

TABLE 6BI
Amino acid sequences of TCR061.

| | | SEQ |
|---|---|---|
| | | ID |
| Description | Sequence | NO: |
| CDR1α | SSNFYA | 1601 |
| CDR2α | MTLNGDE | 1602 |
| CDR3α | AFTTGNQFY | 1603 |
| Vα w/o signal peptide | ILNVEQSPQSLHVQEGDSTNFTCSFPSSNFYALHWYRWETAKSPEALFVMTL | 1604 |
| (SignalP) | NGDEKKKGRISATLNTKEGYSYLYIKGSQPEDSATYLCAFTTGNQFYFGTGT | |
| | SLTVIP | |
| Vα w/o signal peptide | ILNVEQSPQSLHVQEGDSTNFTCSFPSSNFYALHWYRWETAKSPEALFVMTL | 1605 |
| (IMGT) | NGDEKKKGRISATLNTKEGYSYLYIKGSQPEDSATYLCAFTTGNQFYFGTGT | |
| | SLTVIP | |
| Vα | MXKNPLAAPLLILWFHLDCVSSILNVEQSPQSLHVQEGDSTNFTCSFPSSNF | 1606 |
| | YALHWYRWETAKSPEALFVMTLNGDEKKKGRISATLNTKEGYSYLYIKGSQP | 1607 |
| | EDSATYLCAFTTGNQFYFGTGTSLTVIP | |
| | (X = any amino acid) | |
| α chain w/WT signal | MEKNPLAAPLLILWFHLDCVSSILNVEQSPQSLHVQEGDSTNFTCSFPSSNF | 1608 |
| peptide, Cα | YALHWYRWETAKSPEALFVMTLNGDEKKKGRISATLNTKEGYSYLYIKGSQP | |
| (substituted) | EDSATYLCAFTTGNQFYFGTGTSLTVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MAKNPLAAPLLILWFHLDCVSSILNVEQSPQSLHVQEGDSTNFTCSFPSSNF | 1609 |
| signal peptide, Cα | YALHWYRWETAKSPEALFVMTLNGDEKKKGRISATLNTKEGYSYLYIKGSQP | |
| (substituted) | EDSATYLCAFTTGNQFYFGTGTSLTVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MHKNPLAAPLLILWFHLDCVSSILNVEQSPQSLHVQEGDSTNFTCSFPSSNF | 1610 |
| signal peptide, Cα | YALHWYRWETAKSPEALFVMTLNGDEKKKGRISATLNTKEGYSYLYIKGSQP | |
| (substituted) | EDSATYLCAFTTGNQFYFGTGTSLTVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| CDR1β | SGHDYL | 2601 |
| CDR2β | FINNNVP | 2602 |
| CDR3β | ASSSYGGYSNQPQH | 2603 |
| Vβ w/o signal peptide | GVIQSPRHEVTEMGQEVTLRCKPISGHDYLFWYRQTMMRGLELLIYENNNVP | 2604 |
| (SignalP) | IDDSGMPEDRFSAKMPNASFSTLKIQPSEPRDSAVYFCASSSYGGYSNQPQH | |
| | FGDGTRLSIL | |
| Vβ w/o signal peptide | DAGVIQSPRHEVTEMGQEVTLRCKPISGHDYLFWYRQTMMRGLELLIYENNN | 2605 |
| (IMGT) | VPIDDSGMPEDRFSAKMPNASFSTLKIQPSEPRDSAVYFCASSSYGGYSNQP | |
| | QHFGDGTRLSIL | |
| Vβ | MXSWTLCCVSLCILVAKHTDAGVIQSPRHEVTEMGQEVTLRCKPISGHDYLF | 2606 |
| | WYRQTMMRGLELLIYFNNNVPIDDSGMPEDRESAKMPNASFSTLKIQPSEPR | 2607 |
| | DSAVYFCASSSYGGYSNQPQHFGDGTRLSIL | |
| | (X = any amino acid) | |
| β chain w/WT signal | MGSWTLCCVSLCILVAKHTDAGVIQSPRHEVTEMGQEVTLRCKPISGHDYLF | 2608 |
| peptide, Cβ | WYRQTMMRGLELLIYFNNNVPIDDSGMPEDRESAKMPNASFSTLKIQPSEPR | |
| (substituted) | DSAVYFCASSSYGGYSNQPQHFGDGTRLSILEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/alternative | MASWTLCCVSLCILVAKHTDAGVIQSPRHEVTEMGQEVTLRCKPISGHDYLF | 2609 |
| signal peptide, Cβ | WYRQTMMRGLELLIYFNNNVPIDDSGMPEDRESAKMPNASFSTLKIQPSEPR | |
| (substituted) | DSAVYFCASSSYGGYSNQPQHFGDGTRLSILEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/alternative | MHSWTLCCVSLCILVAKHTDAGVIQSPRHEVTEMGQEVTLRCKPISGHDYLE | 2610 |
| signal peptide, Cβ | WYRQTMMRGLELLIYFNNNVPIDDSGMPEDRESAKMPNASESTLKIQPSEPR | |
| (substituted) | DSAVYFCASSSYGGYSNQPQHFGDGTRLSILEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |

In some embodiments, TCR061 interacts with and/or is specific for tumor protein KRAS (KRAS). In some embodiments, the peptide is from a neoantigen of KRAS. In some embodiments, the neoantigen has the amino acid change G12C relative to the wild type KRAS sequence. In some embodiments, TCR061 interacts with the neoantigen in the context of HLA-DRB1*11:01 as described in International Publication No. WO 2019/060349, incorporated herein by reference in its entirety.

TABLE 6BJ
Amino acid sequences of TCR062.

| | | SEQ |
|---|---|---|
| | | ID |
| Description | Sequence | NO: |
| CDR1α | NIATNDY | 1611 |
| CDR2α | GYKTK | 1612 |
| CDR3α | LVGDMDQAGTALI | 1613 |
| Vα w/o signal peptide | KTTQPISMDSYEGQEVNITCSHNNIATNDYITWYQQFPSQGPRFIIQGYKTK | 1614 |
| (SignalP) | VTNEVASLFIPADRKSSTLSLPRVSLSDTAVYYCLVGDMDQAGTALIFGKGT | |
| | TLSVSS | |
| Vα w/o signal peptide | LAKTTQPISMDSYEGQEVNITCSHNNIATNDYITWYQQFPSQGPRFIIQGYK | 1615 |
| (IMGT) | TKVTNEVASLFIPADRKSSTLSLPRVSLSDTAVYYCLVGDMDQAGTALIFGK | |
| | GTTLSVSS | |
| Vα | MXQVARVIVELTLSTLSLAKTTQPISMDSYEGQEVNITCSHNNIATNDYITW | 1616 |
| | YQQFPSQGPRFIIQGYKTKVTNEVASLFIPADRKSSTLSLPRVSLSDTAVYY | 1617 |
| | CLVGDMDQAGTALIFGKGTTLSVSS | |
| | (X = any amino acid) | |
| α chain w/WT signal | MRQVARVIVELTLSTLSLAKTTQPISMDSYEGQEVNITCSHNNIATNDYITW | 1618 |
| peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MAQVARVIVELTLSTLSLAKTTQPISMDSYEGQEVNITCSHNNIATNDYITW | 1619 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MHQVARVIVELTLSTLSLAKTTQPISMDSYEGQEVNITCSHNNIATNDYITW | 1620 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| CDR1β | SGHDT | 2611 |
| CDR2β | YYEEEE | 2612 |
| CDR3β | ASSLGEGRVDGYT | 2613 |
| Vβ w/o signal peptide | GVTQSPTHLIKTRGQQVTLRCSPKSGHDTVSWYQQALGQGPQFIFQYYEEEE | 2614 |
| (SignalP) | RQRGNFPDRFSGHQFPNYSSELNVNALLLGDSALYLCASSLGEGRVDGYTFG | |
| | SGTRLTVV | |
| Vβ w/o signal peptide | DAGVTQSPTHLIKTRGQQVTLRCSPKSGHDTVSWYQQALGQGPQFIFQYYEE | 2615 |
| (IMGT) | EERQRGNFPDRESGHQFPNYSSELNVNALLLGDSALYLCASSLGEGRVDGYT | |
| | FGSGTRLTVV | |
| Vβ | MXPGLLCWALLCLLGAGLVDAGVTQSPTHLIKTRGQQVTLRCSPKSGHDTVS | 2616 |
| | WYQQALGQGPQFIFQYYEEEERQRGNFPDRESGHQFPNYSSELNVNALLLGD | 2617 |
| | SALYLCASSLGEGRVDGYTFGSGTRLTVV | |
| | (X = any amino acid) | |
| β chain w/WT signal | MGPGLLCWALLCLLGAGLVDAGVTQSPTHLIKTRGQQVTLRCSPKSGHDTVS | 2618 |
| peptide, Cβ | WYQQALGQGPQFIFQYYEEEERQRGNFPDRFSGHQFPNYSSELNVNALLLGD | |
| (substituted) | SALYLCASSLGEGRVDGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MAPGLLCWALLCLLGAGLVDAGVTQSPTHLIKTRGQQVTLRCSPKSGHDTVS | 2619 |
| signal peptide, Cβ | WYQQALGQGPQFIFQYYEEEERQRGNFPDRESGHQFPNYSSELNVNALLLGD | |
| (substituted) | SALYLCASSLGEGRVDGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MHPGLLCWALLCLLGAGLVDAGVTQSPTHLIKTRGQQVTLRCSPKSGHDTVS | 2620 |
| signal peptide, Cβ | WYQQALGQGPQFIFQYYEEEERQRGNFPDRESGHQFPNYSSELNVNALLLGD | |
| (substituted) | SALYLCASSLGEGRVDGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR062 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR062 interacts with the neoantigen in the context of HLA-C*08:02 as described in International Publication No. WO 2018/026691, incorporated herein by reference in its entirety.

TABLE 6BK
Amino acid sequences of TCR063.

| | | SEQ |
|---|---|---|
| | | ID |
| Description | Sequence | NO: |
| CDR1α | NIATNDY | 1621 |
| CDR2α | GYKTK | 1622 |
| CDR3α | LVGDMDQAGTALI | 1623 |
| Vα w/o signal peptide | KTTQPISMDSYEGQEVNITCSHNNIATNDYITWYQQFPSQGPRFIIQGYKTK | 1624 |
| (SignalP) | VTNEVASLFIPADRKSSTLSLPRVSLSDTAVYYCLVGDMDQAGTALIFGKGT | |
| | TLSVSS | |
| Vα w/o signal peptide | LAKTTQPISMDSYEGQEVNITCSHNNIATNDYITWYQQFPSQGPRFIIQGYK | 1625 |
| (IMGT) | TKVTNEVASLFIPADRKSSTLSLPRVSLSDTAVYYCLVGDMDQAGTALIFGK | |
| | GTTLSVSS | |
| Vα | MXQVARVIVELTLSTLSLAKTTQPISMDSYEGQEVNITCSHNNIATNDYITW | 1626 |
| | YQQFPSQGPRFIIQGYKTKVTNEVASLFIPADRKSSTLSLPRVSLSDTAVYY | 1627 |
| | CLVGDMDQAGTALIFGKGTTLSVSS | |
| | (X = any amino acid) | |
| α chain w/WT signal | MRQVARVIVELTLSTLSLAKTTQPISMDSYEGQEVNITCSHNNIATNDYITW | 1628 |
| peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MAQVARVIVELTLSTLSLAKTTQPISMDSYEGQEVNITCSHNNIATNDYITW | 1629 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MHQVARVIVELTLSTLSLAKTTQPISMDSYEGQEVNITCSHNNIATNDYITW | 1630 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| CDR1β | SGHDT | 2621 |
| CDR2β | YYEEEE | 2622 |
| CDR3β | ASSLGRASNQPQH | 2623 |
| Vβ w/o signal peptide | GVTQSPTHLIKTRGQQVTLRCSPKSGHDTVSWYQQALGQGPQFIFQYYEEEE | 2624 |
| (SignalP) | RQRGNFPDRFSGHQFPNYSSELNVNALLLGDSALYLCASSLGRASNQPQHFG | |
| | DGTRLSIL | |
| Vβ w/o signal peptide | DAGVTQSPTHLIKTRGQQVTLRCSPKSGHDTVSWYQQALGQGPQFIFQYYEE | 2625 |
| (IMGT) | EERQRGNFPDRFSGHQFPNYSSELNVNALLLGDSALYLCASSLGRASNQPQH | |
| | FGDGTRLSIL | |
| Vβ | MXPGLLCWALLCLLGAGLVDAGVTQSPTHLIKTRGQQVTLRCSPKSGHDTVS | 2626 |
| | WYQQALGQGPQFIFQYYEEEERQRGNFPDRESGHQFPNYSSELNVNALLLGD | 2627 |
| | SALYLCASSLGRASNQPQHFGDGTRLSIL | |
| | (X = any amino acid) | |
| β chain w/WT signal | MGPGLLCWALLCLLGAGLVDAGVTQSPTHLIKTRGQQVTLRCSPKSGHDTVS | 2628 |
| peptide, Cβ | WYQQALGQGPQFIFQYYEEEERQRGNFPDRESGHQFPNYSSELNVNALLLGD | |
| (substituted) | SALYLCASSLGRASNQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MAPGLLCWALLCLLGAGLVDAGVTQSPTHLIKTRGQQVTLRCSPKSGHDTVS | 2629 |
| signal peptide, Cβ | WYQQALGQGPQFIFQYYEEEERQRGNFPDRESGHQFPNYSSELNVNALLLGD | |
| (substituted) | SALYLCASSLGRASNQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MHPGLLCWALLCLLGAGLVDAGVTQSPTHLIKTRGQQVTLRCSPKSGHDTVS | 2630 |
| signal peptide, Cβ | WYQQALGQGPQFIFQYYEEEERQRGNFPDRFSGHQFPNYSSELNVNALLLGD | |
| (substituted) | SALYLCASSLGRASNQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR063 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR063 interacts with the neoantigen in the context of HLA-C*08:02 as described in International Publication No. WO 2018/026691, incorporated herein by reference in its entirety.

TABLE 6BL
Amino acid sequences of TCR064.

| | | SEQ |
|---|---|---|
| | | ID |
| Description | Sequence | NO: |
| CDR1α | NIATNDY | 1631 |
| CDR2α | GYKTK | 1632 |
| CDR3α | LVGDRDQAGTALI | 1633 |
| Vα w/o signal peptide | KTTQPISMDSYEGQEVNITCSHNNIATNDYITWYQQFPSQGPRFIIQGYKTK | 1634 |
| (SignalP) | VTNEVASLFIPADRKSSTLSLPRVSLSDTAVYYCLVGDRDQAGTALIFGKGT | |
| | TLSVSS | |
| Vα w/o signal peptide | LAKTTQPISMDSYEGQEVNITCSHNNIATNDYITWYQQFPSQGPRFIIQGYK | 1635 |
| (IMGT) | TKVTNEVASLFIPADRKSSTLSLPRVSLSDTAVYYCLVGDRDQAGTALIFGK | |
| | GTTLSVSS | |
| Vα | MXQVARVIVELTLSTLSLAKTTQPISMDSYEGQEVNITCSHNNIATNDYITW | 1636 |
| | YQQFPSQGPRFIIQGYKTKVTNEVASLFIPADRKSSTLSLPRVSLSDTAVYY | 1637 |
| | CLVGDRDQAGTALIFGKGTTLSVSS | |
| | (X = any amino acid) | |
| α chain w/WT signal | MRQVARVIVELTLSTLSLAKTTQPISMDSYEGQEVNITCSHNNIATNDYITW | 1638 |
| peptide, Cα | YQQFPSQGPRFIIQGYKTKVTNEVASLFIPADRKSSTLSLPRVSLSDTAVYY | |
| (substituted) | CLVGDRDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MAQVARVIVELTLSTLSLAKTTQPISMDSYEGQEVNITCSHNNIATNDYITW | 1639 |
| signal peptide, Cα | YQQFPSQGPRFIIQGYKTKVTNEVASLFIPADRKSSTLSLPRVSLSDTAVYY | |
| (substituted) | CLVGDRDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MHQVARVIVFLTLSTLSLAKTTQPISMDSYEGQEVNITCSHNNIATNDYITW | 1640 |
| signal peptide, Cα | YQQFPSQGPRFIIQGYKTKVTNEVASLFIPADRKSSTLSLPRVSLSDTAVYY | |
| (substituted) | CLVGDRDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| CDR1β | SGHDT | 2631 |
| CDR2β | YYEEEE | 2632 |
| CDR3β | ASSFGQSSTYGYT | 2633 |
| Vβ w/o signal peptide | GVTQSPTHLIKTRGQQVTLRCSPKSGHDTVSWYQQALGQGPQFIFQYYEEEE | 2634 |
| (SignalP) | RQRGNFPDRESGHQFPNYSSELNVNALLLGDSALYLCASSFGQSSTYGYTFG | |
| | SGTRLTVV | |
| Vβ w/o signal peptide | DAGVTQSPTHLIKTRGQQVTLRCSPKSGHDTVSWYQQALGQGPQFIFQYYEE | 2635 |
| (IMGT) | EERQRGNFPDRESGHQFPNYSSELNVNALLLGDSALYLCASSFGQSSTYGYT | |
| | FGSGTRLTVV | |
| Vβ | MXPGLLCWALLCLLGAGLVDAGVTQSPTHLIKTRGQQVTLRCSPKSGHDTVS | 2636 |
| | WYQQALGQGPQFIFQYYEEEERQRGNFPDRFSGHQFPNYSSELNVNALLLGD | 2637 |
| | SALYLCASSFGQSSTYGYTFGSGTRLTVV | |
| | (X = any amino acid) | |
| β chain w/WT signal | MGPGLLCWALLCLLGAGLVDAGVTQSPTHLIKTRGQQVTLRCSPKSGHDTVS | 2638 |
| peptide, Cβ | WYQQALGQGPQFIFQYYEEEERQRGNFPDRESGHQFPNYSSELNVNALLLGD | |
| (substituted) | SALYLCASSFGQSSTYGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MAPGLLCWALLCLLGAGLVDAGVTQSPTHLIKTRGQQVTLRCSPKSGHDTVS | 2639 |
| signal peptide, Cβ | WYQQALGQGPQFIFQYYEEEERQRGNFPDRESGHQFPNYSSELNVNALLLGD | |
| (substituted) | SALYLCASSFGQSSTYGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MHPGLLCWALLCLLGAGLVDAGVTQSPTHLIKTRGQQVTLRCSPKSGHDTVS | 2640 |
| signal peptide, Cβ | WYQQALGQGPQFIFQYYEEEERQRGNFPDRFSGHQFPNYSSELNVNALLLGD | |
| (substituted) | SALYLCASSFGQSSTYGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR064 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR064 interacts with the neoantigen in the context of HLA-C*08:02 as described in International Publication No. WO 2018/026691, incorporated herein by reference in its entirety.

TABLE 6BM
Amino acid sequences of TCR065.

| | | SEQ |
|---|---|---|
| | | ID |
| Description | Sequence | NO: |
| CDR1α | NIATNDY | 1641 |
| CDR2α | GYKTK | 1642 |
| CDR3α | LVGDMDQAGTALI | 1643 |
| Vα w/o signal peptide | KTTQPISMDSYEGQEVNITCSHNNIATNDYITWYQQFPSQGPRFIIQGYKTK | 1644 |
| (SignalP) | VTNEVASLFIPADRKSSTLSLPRVSLSDTAVYYCLVGDMDQAGTALIFGKGT | |
| | TLSVSS | |
| Vα w/o signal peptide | LAKTTQPISMDSYEGQEVNITCSHNNIATNDYITWYQQFPSQGPRFIIQGYK | 1645 |
| (IMGT) | TKVTNEVASLFIPADRKSSTLSLPRVSLSDTAVYYCLVGDMDQAGTALIFGK | |
| | GTTLSVSS | |
| Vα | MXQVARVIVELTLSTLSLAKTTQPISMDSYEGQEVNITCSHNNIATNDYITW | 1646 |
| | YQQFPSQGPRFIIQGYKTKVTNEVASLFIPADRKSSTLSLPRVSLSDTAVYY | 1647 |
| | CLVGDMDQAGTALIFGKGTTLSVSS | |
| | (X = any amino acid) | |
| α chain w/WT signal | MRQVARVIVELTLSTLSLAKTTQPISMDSYEGQEVNITCSHNNIATNDYITW | 1648 |
| peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MAQVARVIVELTLSTLSLAKTTQPISMDSYEGQEVNITCSHNNIATNDYITW | 1649 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/alternative | MHQVARVIVELTLSTLSLAKTTQPISMDSYEGQEVNITCSHNNIATNDYITW | 1650 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| CDR1β | SGHDT | 2641 |
| CDR2β | YYEEEE | 2642 |
| CDR3β | ASSLGQTNYGYT | 2643 |
| Vβ w/o signal peptide | GVTQSPTHLIKTRGQQVTLRCSPKSGHDTVSWYQQALGQGPQFIFQYYEEEE | 2644 |
| (SignalP) | RQRGNFPDRESGHQFPNYSSELNVNALLLGDSALYLCASSLGQTNYGYTFGS | |
| | GTRLTVV | |
| Vβ w/o signal peptide | DAGVTQSPTHLIKTRGQQVTLRCSPKSGHDTVSWYQQALGQGPQFIFQYYEE | 2645 |
| (IMGT) | EERQRGNFPDRESGHQFPNYSSELNVNALLLGDSALYLCASSLGQTNYGYTE | |
| | GSGTRLTVV | |
| Vβ | MXPGLLCWALLCLLGAGLVDAGVTQSPTHLIKTRGQQVTLRCSPKSGHDTVS | 2646 |
| | WYQQALGQGPQFIFQYYEEEERQRGNFPDRFSGHQFPNYSSELNVNALLLGD | 2647 |
| | SALYLCASSLGQTNYGYTFGSGTRLTVV | |
| | (X = any amino acid) | |
| β chain w/WT signal | MGPGLLCWALLCLLGAGLVDAGVTQSPTHLIKTRGQQVTLRCSPKSGHDTVS | 2648 |
| peptide, Cβ | WYQQALGQGPQFIFQYYEEEERQRGNFPDRESGHQFPNYSSELNVNALLLGD | |
| (substituted) | SALYLCASSLGQTNYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAPGLLCWALLCLLGAGLVDAGVTQSPTHLIKTRGQQVTLRCSPKSGHDTVS | 2649 |
| signal peptide, Cβ | WYQQALGQGPQFIFQYYEEEERQRGNFPDRFSGHQFPNYSSELNVNALLLGD | |
| (substituted) | SALYLCASSLGQTNYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHPGLLCWALLCLLGAGLVDAGVTQSPTHLIKTRGQQVTLRCSPKSGHDTVS | 2650 |
| signal peptide, Cβ | WYQQALGQGPQFIFQYYEEEERQRGNFPDRESGHQFPNYSSELNVNALLLGD | |
| (substituted) | SALYLCASSLGQTNYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR065 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR065 interacts with the neoantigen in the context of HLA-Cw*08:02 as described in International Publication No. WO 2017/048593, incorporated herein by reference in its entirety.

TABLE 6BN
Amino acid sequences of TCR066.

| | | SEQ |
|---|---|---|
| | | ID |
| Description | Sequence | NO: |
| CDR1α | DRGSQS | 1651 |
| CDR2α | IYSNGD | 1652 |
| CDR3α | AAAMDSSYKLI | 1653 |
| Vα w/o signal peptide | QQKEVEQNSGPLSVPEGAIASLNCTYSDRGSQSFFWYRQYSGKSPELIMFIY | 1654 |
| (SignalP) | SNGDKEDGRFTAQLNKASQYVSLLIRDSQPSDSATYLCAAAMDSSYKLIFGS | |
| | GTRLLVRP | |
| Vα w/o signal peptide | QKEVEQNSGPLSVPEGAIASLNCTYSDRGSQSFFWYRQYSGKSPELIMFIYS | 1655 |
| (IMGT) | NGDKEDGRFTAQLNKASQYVSLLIRDSQPSDSATYLCAAAMDSSYKLIFGSG | |
| | TRLLVRP | |
| Vα | MXSLRVLLVILWLQLSWVWSQQKEVEQNSGPLSVPEGAIASLNCTYSDRGSQ | 1656 |
| | SFFWYRQYSGKSPELIMFIYSNGDKEDGRFTAQLNKASQYVSLLIRDSQPSD | 1657 |
| | SATYLCAAAMDSSYKLIFGSGTRLLVRP | |
| | (X = any amino acid) | |
| α chain w/WT signal | MKSLRVLLVILWLQLSWVWSQQKEVEQNSGPLSVPEGAIASLNCTYSDRGSQ | 1658 |
| peptide, Cα | SFFWYRQYSGKSPELIMFIYSNGDKEDGRFTAQLNKASQYVSLLIRDSQPSD | |
| (substituted) | SATYLCAAAMDSSYKLIFGSGTRLLVRPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MASLRVLLVILWLQLSWVWSQQKEVEQNSGPLSVPEGAIASLNCTYSDRGSQ | 1659 |
| signal peptide, Cα | SFFWYRQYSGKSPELIMFIYSNGDKEDGRFTAQLNKASQYVSLLIRDSQPSD | |
| (substituted) | SATYLCAAAMDSSYKLIFGSGTRLLVRPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MHSLRVLLVILWLQLSWVWSQQKEVEQNSGPLSVPEGAIASLNCTYSDRGSQ | 1660 |
| signal peptide, Cα | SFFWYRQYSGKSPELIMFIYSNGDKEDGRETAQLNKASQYVSLLIRDSQPSD | |
| (substituted) | SATYLCAAAMDSSYKLIFGSGTRLLVRPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| CDR1β | WSHSY | 2651 |
| CDR2β | SAAADI | 2652 |
| CDR3β | ASSDPGTEAF | 2653 |
| Vβ w/o signal peptide | GITQSPRYKITETGRQVTLMCHQTWSHSYMFWYRQDLGHGLRLIYYSAAADI | 2654 |
| (SignalP) | TDKGEVPDGYVVSRSKTENFPLTLESATRSQTSVYFCASSDPGTEAFFGQGT | |
| | RLTVV | |
| Vβ w/o signal peptide | DAGITQSPRYKITETGRQVTLMCHQTWSHSYMFWYRQDLGHGLRLIYYSAAA | 2655 |
| (IMGT) | DITDKGEVPDGYVVSRSKTENFPLTLESATRSQTSVYFCASSDPGTEAFFGQ | |
| | GTRLTVV | |
| Vβ | MXTRLFFYVALCLLWAGHRDAGITQSPRYKITETGRQVTLMCHQTWSHSYMF | 2656 |
| | WYRQDLGHGLRLIYYSAAADITDKGEVPDGYVVSRSKTENFPLTLESATRSQ | 2657 |
| | TSVYFCASSDPGTEAFFGQGTRLTVV | |
| | (X = any amino acid) | |
| ß chain w/WT signal | MGTRLFFYVALCLLWAGHRDAGITQSPRYKITETGRQVTLMCHQTWSHSYMF | 2658 |
| peptide, Cβ | WYRQDLGHGLRLIYYSAAADITDKGEVPDGYVVSRSKTENFPLTLESATRSQ | |
| (substituted) | TSVYFCASSDPGTEAFFGQGTRLTVVEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain w/alternative | MATRLFFYVALCLLWAGHRDAGITQSPRYKITETGRQVTLMCHQTWSHSYMF | 2659 |
| signal peptide, Cβ | WYRQDLGHGLRLIYYSAAADITDKGEVPDGYVVSRSKTENFPLTLESATRSQ | |
| (substituted) | TSVYFCASSDPGTEAFFGQGTRLTVVEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain w/alternative | MHTRLFFYVALCLLWAGHRDAGITQSPRYKITETGRQVTLMCHQTWSHSYMF | 2660 |
| signal peptide, Cβ | WYRQDLGHGLRLIYYSAAADITDKGEVPDGYVVSRSKTENFPLTLESATRSQ | |
| (substituted) | TSVYFCASSDPGTEAFFGQGTRLTVVEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR066 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR066 interacts with the neoantigen in the context of HLA-C*08:02 as described in International Publication No. WO 2018/026691, incorporated herein by reference in its entirety.

TABLE 6BO
Amino acid sequences of TCR067.

| | | SEQ |
|---|---|---|
| | | ID |
| Description | Sequence | NO: |
| CDR1α | TIYSNPF | 1661 |
| CDR2α | SFTDNKR | 1662 |
| CDR3α | ALRGNAGAKLT | 1663 |
| Vα w/o signal peptide | DGDSVTQTEGLVTLTEGLPVMLNCTYQTIYSNPFLFWYVQHLNESPRLLLKS | 1664 |
| (SignalP) | FTDNKRTEHQGFHATLHKSSSSFHLQKSSAQLSDSALYYCALRGNAGAKLTE | |
| | GGGTRLTVRPD | |
| Vα w/o signal peptide | GDSVTQTEGLVTLTEGLPVMLNCTYQTIYSNPFLFWYVQHLNESPRLLLKSF | 1665 |
| (IMGT) | TDNKRTEHQGFHATLHKSSSSFHLQKSSAQLSDSALYYCALRGNAGAKLTFG | |
| | GGTRLTVRPD | |
| Vα | MXPVTCSVLVLLLMLRRSNGDGDSVTQTEGLVTLTEGLPVMLNCTYQTIYSN | 1666 |
| | PFLFWYVQHLNESPRLLLKSFTDNKRTEHQGFHATLHKSSSSFHLQKSSAQL | 1667 |
| | SDSALYYCALRGNAGAKLTFGGGTRLTVRPD | |
| | (X = any amino acid) | |
| α chain w/WT signal | MRPVTCSVLVLLLMLRRSNGDGDSVTQTEGLVTLTEGLPVMLNCTYQTIYSN | 1668 |
| peptide, Cα | PFLFWYVQHLNESPRLLLKSFTDNKRTEHQGFHATLHKSSSSFHLQKSSAQL | |
| (substituted) | SDSALYYCALRGNAGAKLTFGGGTRLTVRPDIQNPEPAVYQLKDPRSQDSTL | |
| | CLFTDFDSQINVPKTMESGTFITDKTVLDMKAMDSKSNGAIAWSNQTSFTCQ | |
| | DIFKETNATYPSSDVPCDATLTEKSFETDMNLNFQNLSVMGLRILLLKVAGE | |
| | NLLMTLRLWSS | |
| α chain w/alternative | MAPVTCSVLVLLLMLRRSNGDGDSVTQTEGLVTLTEGLPVMLNCTYQTIYSN | 1669 |
| signal peptide, Cα | PFLFWYVQHLNESPRLLLKSFTDNKRTEHQGFHATLHKSSSSFHLQKSSAQL | |
| (substituted) | SDSALYYCALRGNAGAKLTFGGGTRLTVRPDIQNPEPAVYQLKDPRSQDSTL | |
| | CLFTDFDSQINVPKTMESGTFITDKTVLDMKAMDSKSNGAIAWSNQTSFTCQ | |
| | DIFKETNATYPSSDVPCDATLTEKSFETDMNLNFQNLSVMGLRILLLKVAGF | |
| | NLLMTLRLWSS | |
| α chain w/alternative | MHPVTCSVLVLLLMLRRSNGDGDSVTQTEGLVTLTEGLPVMLNCTYQTIYSN | 1670 |
| signal peptide, Cα | PFLFWYVQHLNESPRLLLKSFTDNKRTEHQGFHATLHKSSSSFHLQKSSAQL | |
| (substituted) | SDSALYYCALRGNAGAKLTFGGGTRLTVRPDIQNPEPAVYQLKDPRSQDSTL | |
| | CLFTDFDSQINVPKTMESGTFITDKTVLDMKAMDSKSNGAIAWSNQTSFTCQ | |
| | DIFKETNATYPSSDVPCDATLTEKSFETDMNLNFQNLSVMGLRILLLKVAGF | |
| | NLLMTLRLWSS | |
| CDR1ß | LGHDT | 2661 |
| CDR2ß | YNNKQL | 2662 |
| CDR3ß | ASSSRDWSAETLY | 2663 |
| Vß w/o signal peptide | AVFQTPNYHVTQVGNEVSFNCKQTLGHDTMYWYKQDSKKLLKIMFSYNNKQL | 2664 |
| (SignalP) | IVNETVPRRFSPQSSDKAHLNLRIKSVEPEDSAVYLCASSSRDWSAETLYFG | |
| | SGTRLTVL | |
| Vß w/o signal peptide | ETAVFQTPNYHVTQVGNEVSFNCKQTLGHDTMYWYKQDSKKLLKIMFSYNNK | 2665 |
| (IMGT) | QLIVNETVPRRESPQSSDKAHLNLRIKSVEPEDSAVYLCASSSRDWSAETLY | |
| | FGSGTRLTVL | |
| Vß | MXCRLLSCVAFCLLGIGPLETAVFQTPNYHVTQVGNEVSENCKQTLGHDTMY | 2666 |
| | WYKQDSKKLLKIMFSYNNKQLIVNETVPRRFSPQSSDKAHLNLRIKSVEPED | 2667 |
| | SAVYLCASSSRDWSAETLYFGSGTRLTVL | |
| | (X = any amino acid) | |
| ß chain w/WT signal | MGCRLLSCVAFCLLGIGPLETAVFQTPNYHVTQVGNEVSFNCKQTLGHDTMY | 2668 |
| peptide, Cß | WYKQDSKKLLKIMFSYNNKQLIVNETVPRRFSPQSSDKAHLNLRIKSVEPED | |
| (substituted) | SAVYLCASSSRDWSAETLYFGSGTRLTVLEDLRNVTPPKVSLFEPSKAEIAN | |
| | KQKATLVCLARGFFPDHVELSWWVNGKEVHSGVSTDPQAYKESNYSYCLSSR | |
| | LRVSATFWHNPRNHFRCQVQFHGLSEEDKWPEGSPKPVTQNISAEAWGRADC | |
| | GITSASYHQGVLSATILYEILLGKATLYAVLVSGLVLMAMVKKKNS | |
| ß chain w/alternative | MACRLLSCVAFCLLGIGPLETAVFQTPNYHVTQVGNEVSFNCKQTLGHDTMY | 2669 |
| signal peptide, Cß | WYKQDSKKLLKIMFSYNNKQLIVNETVPRRFSPQSSDKAHLNLRIKSVEPED | |
| (substituted) | SAVYLCASSSRDWSAETLYFGSGTRLTVLEDLRNVTPPKVSLFEPSKAEIAN | |
| | KQKATLVCLARGFFPDHVELSWWVNGKEVHSGVSTDPQAYKESNYSYCLSSR | |
| | LRVSATFWHNPRNHFRCQVQFHGLSEEDKWPEGSPKPVTQNISAEAWGRADC | |
| | GITSASYHQGVLSATILYEILLGKATLYAVLVSGLVLMAMVKKKNS | |
| ß chain w/alternative | MHCRLLSCVAFCLLGIGPLETAVFQTPNYHVTQVGNEVSFNCKQTLGHDTMY | 2670 |
| signal peptide, Cß | WYKQDSKKLLKIMFSYNNKQLIVNETVPRRFSPQSSDKAHLNLRIKSVEPED | |
| (substituted) | SAVYLCASSSRDWSAETLYFGSGTRLTVLEDLRNVTPPKVSLFEPSKAEIAN | |
| | KQKATLVCLARGFFPDHVELSWWVNGKEVHSGVSTDPQAYKESNYSYCLSSR | |
| | LRVSATFWHNPRNHFRCQVQFHGLSEEDKWPEGSPKPVTQNISAEAWGRADC | |
| | GITSASYHQGVLSATILYEILLGKATLYAVLVSGLVLMAMVKKKNS | |

In some embodiments, TCR067 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR067 interacts with the neoantigen in the context of HLA-A11, as described in International Publication No. WO 2016/085904, incorporated herein by reference in its entirety.

TABLE 6BP
Amino acid sequences of TCR068.

| | | SEQ |
|---|---|---|
| | | ID |
| Description | Sequence | NO: |
| CDR1α | DPNSYY | 1671 |
| CDR2α | VFSSTEI | 1672 |
| CDR3α | AVSGGTNSAGNKLT | 1673 |
| Vα w/o signal peptide | EQVEQRPPHLSVREGDSAVITCTYTDPNSYYFFWYKQEPGASLQLLMKVESS | 1674 |
| (SignalP) | TEINEGQGFTVLLNKKDKRLSLNLTAAHPGDSAAYFCAVSGGTNSAGNKLTF | |
| | GIGTRVLVRP | |
| Vα w/o signal peptide | GEQVEQRPPHLSVREGDSAVITCTYTDPNSYYFFWYKQEPGASLQLLMKVFS | 1675 |
| (IMGT) | STEINEGQGFTVLLNKKDKRLSLNLTAAHPGDSAAYFCAVSGGTNSAGNKLT | |
| | FGIGTRVLVRP | |
| Vα | MXTVTGPLFLCFWLQLNCVSRGEQVEQRPPHLSVREGDSAVITCTYTDPNSY | 1676 |
| | YFFWYKQEPGASLQLLMKVFSSTEINEGQGFTVLLNKKDKRLSLNLTAAHPG | 1677 |
| | DSAAYFCAVSGGTNSAGNKLTFGIGTRVLVRP | |
| | (X = any amino acid) | |
| α chain w/WT signal | MKTVTGPLFLCFWLQLNCVSRGEQVEQRPPHLSVREGDSAVITCTYTDPNSY | 1678 |
| peptide, Cα | YFFWYKQEPGASLQLLMKVFSSTEINEGQGFTVLLNKKDKRLSLNLTAAHPG | |
| (substituted) | DSAAYFCAVSGGTNSAGNKLTFGIGTRVLVRPDIQNPEPAVYQLKDPRSQDS | |
| | TLCLFTDFDSQINVPKTMESGTFITDKTVLDMKAMDSKSNGAIAWSNQTSFT | |
| | CQDIFKETNATYPSSDVPCDATLTEKSFETDMNLNFQNLSVMGLRILLLKVA | |
| | GFNLLMTLRLWSS | |
| α chain w/alternative | MATVTGPLFLCFWLQLNCVSRGEQVEQRPPHLSVREGDSAVITCTYTDPNSY | 1679 |
| signal peptide, Cα | YFFWYKQEPGASLQLLMKVFSSTEINEGQGFTVLLNKKDKRLSLNLTAAHPG | |
| (substituted) | DSAAYFCAVSGGTNSAGNKLTFGIGTRVLVRPDIQNPEPAVYQLKDPRSQDS | |
| | TLCLFTDFDSQINVPKTMESGTFITDKTVLDMKAMDSKSNGAIAWSNQTSFT | |
| | CQDIFKETNATYPSSDVPCDATLTEKSFETDMNLNFQNLSVMGLRILLLKVA | |
| | GENLLMTLRLWSS | |
| α chain w/alternative | MHTVTGPLFLCFWLQLNCVSRGEQVEQRPPHLSVREGDSAVITCTYTDPNSY | 1680 |
| signal peptide, Cα | YFFWYKQEPGASLQLLMKVFSSTEINEGQGFTVLLNKKDKRLSLNLTAAHPG | |
| (substituted) | DSAAYFCAVSGGTNSAGNKLTFGIGTRVLVRPDIQNPEPAVYQLKDPRSQDS | |
| | TLCLFTDFDSQINVPKTMESGTFITDKTVLDMKAMDSKSNGAIAWSNQTSFT | |
| | CQDIFKETNATYPSSDVPCDATLTEKSFETDMNLNFQNLSVMGLRILLLKVA | |
| | GFNLLMTLRLWSS | |
| CDR1ß | LGHDT | 2671 |
| CDR2ß | YNNKQL | 2672 |
| CDR3ß | ASSRDWGPAEQF | 2673 |
| Vß w/o signal peptide | AVFQTPNYHVTQVGNEVSFNCKQTLGHDTMYWYKQDSKKLLKIMFSYNNKQL | 2674 |
| (SignalP) | IVNETVPRRESPQSSDKAHLNLRIKSVEPEDSAVYLCASSRDWGPAEQFFGP | |
| | GTRLTVL | |
| Vß w/o signal peptide | ETAVFQTPNYHVTQVGNEVSFNCKQTLGHDTMYWYKQDSKKLLKIMFSYNNK | 2675 |
| (IMGT) | QLIVNETVPRRFSPQSSDKAHLNLRIKSVEPEDSAVYLCASSRDWGPAEQFF | |
| | GPGTRLTVL | |
| Vß | MXCRLLSCVAFCLLGIGPLETAVFQTPNYHVTQVGNEVSFNCKQTLGHDTMY | 2676 |
| | WYKQDSKKLLKIMFSYNNKQLIVNETVPRRFSPQSSDKAHLNLRIKSVEPED | 2677 |
| | SAVYLCASSRDWGPAEQFFGPGTRLTVL | |
| | (X = any amino acid) | |
| ß chain w/WT signal | MGCRLLSCVAFCLLGIGPLETAVFQTPNYHVTQVGNEVSFNCKQTLGHDTMY | 2678 |
| peptide, Cß | WYKQDSKKLLKIMFSYNNKQLIVNETVPRRFSPQSSDKAHLNLRIKSVEPED | |
| (substituted) | SAVYLCASSRDWGPAEQFFGPGTRLTVLEDLRNVTPPKVSLFEPSKAEIANK | |
| | QKATLVCLARGFFPDHVELSWWVNGKEVHSGVSTDPQAYKESNYSYCLSSRL | |
| | RVSATFWHNPRNHFRCQVQFHGLSEEDKWPEGSPKPVTQNISAEAWGRADCG | |
| | ITSASYHQGVLSATILYEILLGKATLYAVLVSGLVLMAMVKKKNS | |
| ß chain w/alternative | MACRLLSCVAFCLLGIGPLETAVFQTPNYHVTQVGNEVSFNCKQTLGHDTMY | 2679 |
| signal peptide, Cß | WYKQDSKKLLKIMFSYNNKQLIVNETVPRRFSPQSSDKAHLNLRIKSVEPED | |
| (substituted) | SAVYLCASSRDWGPAEQFFGPGTRLTVLEDLRNVTPPKVSLFEPSKAEIANK | |
| | QKATLVCLARGFFPDHVELSWWVNGKEVHSGVSTDPQAYKESNYSYCLSSRL | |
| | RVSATFWHNPRNHFRCQVQFHGLSEEDKWPEGSPKPVTQNISAEAWGRADCG | |
| | ITSASYHQGVLSATILYEILLGKATLYAVLVSGLVLMAMVKKKNS | |
| ß chain w/alternative | MHCRLLSCVAFCLLGIGPLETAVFQTPNYHVTQVGNEVSFNCKQTLGHDTMY | 2680 |
| signal peptide, Cß | WYKQDSKKLLKIMFSYNNKQLIVNETVPRRFSPQSSDKAHLNLRIKSVEPED | |
| (substituted) | SAVYLCASSRDWGPAEQFFGPGTRLTVLEDLRNVTPPKVSLFEPSKAEIANK | |
| | QKATLVCLARGFFPDHVELSWWVNGKEVHSGVSTDPQAYKESNYSYCLSSRL | |
| | RVSATFWHNPRNHFRCQVQFHGLSEEDKWPEGSPKPVTQNISAEAWGRADCG | |
| | ITSASYHQGVLSATILYEILLGKATLYAVLVSGLVLMAMVKKKNS | |

In some embodiments, TCR068 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR068 interacts with the neoantigen in the context of HLA-A11, as described in International Publication No. WO 2016/085904, incorporated herein by reference in its entirety.

TABLE 6BQ
Amino acid sequences of TCR069.

| | | SEQ |
|---|---|---|
| | | ID |
| Description | Sequence | NO: |
| CDR1α | NDMEDY | 1681 |
| CDR2α | VRSNVDK | 1682 |
| CDR3α | AAGDSGGSNYKLT | 1683 |
| Vα w/o signal peptide | QQKTGGQQVKQSSPSLTVQEGGILILNCDYENDMEDYFAWYKKYPDNSPTLL | 1684 |
| (SignalP) | ISVRSNVDKREDGRFTVFLNKSGKHFSLHITASQPEDTAVYLCAAGDSGGSN | |
| | YKLTFGKGTLLTVTP | |
| Vα w/o signal peptide | QQKTGGQQVKQSSPSLTVQEGGILILNCDYENDMEDYFAWYKKYPDNSPTLL | 1685 |
| (IMGT) | ISVRSNVDKREDGRFTVFLNKSGKHFSLHITASQPEDTAVYLCAAGDSGGSN | |
| | YKLTFGKGTLLTVTP | |
| Vα | MXGFLKALLLVLCLRPEWIKSQQKTGGQQVKQSSPSLTVQEGGILILNCDYE | 1686 |
| | NDMFDYFAWYKKYPDNSPTLLISVRSNVDKREDGRFTVFLNKSGKHFSLHIT | 1687 |
| | ASQPEDTAVYLCAAGDSGGSNYKLTFGKGTLLTVTP | |
| | (X = any amino acid) | |
| α chain w/WT signal | MTGFLKALLLVLCLRPEWIKSQQKTGGQQVKQSSPSLTVQEGGILILNCDYE | 1688 |
| peptide, Cα | NDMFDYFAWYKKYPDNSPTLLISVRSNVDKREDGRFTVFLNKSGKHFSLHIT | |
| (substituted) | ASQPEDTAVYLCAAGDSGGSNYKLTFGKGTLLTVTPNIQNPEPAVYQLKDPR | |
| | SQDSTLCLFTDFDSQINVPKTMESGTFITDKTVLDMKAMDSKSNGAIAWSNQ | |
| | TSFTCQDIFKETNATYPSSDVPCDATLTEKSFETDMNLNFQNLSVMGLRILL | |
| | LKVAGFNLLMTLRLWSS | |
| α chain w/alternative | MAGFLKALLLVLCLRPEWIKSQQKTGGQQVKQSSPSLTVQEGGILILNCDYE | 1689 |
| signal peptide, Cα | NDMFDYFAWYKKYPDNSPTLLISVRSNVDKREDGRFTVFLNKSGKHFSLHIT | |
| (substituted) | ASQPEDTAVYLCAAGDSGGSNYKLTFGKGTLLTVTPNIQNPEPAVYQLKDPR | |
| | SQDSTLCLFTDFDSQINVPKTMESGTFITDKTVLDMKAMDSKSNGAIAWSNQ | |
| | TSFTCQDIFKETNATYPSSDVPCDATLTEKSFETDMNLNFQNLSVMGLRILL | |
| | LKVAGFNLLMTLRLWSS | |
| α chain w/alternative | MHGFLKALLLVLCLRPEWIKSQQKTGGQQVKQSSPSLTVQEGGILILNCDYE | 1690 |
| signal peptide, Cα | NDMEDYFAWYKKYPDNSPTLLISVRSNVDKREDGRFTVFLNKSGKHFSLHIT | |
| (substituted) | ASQPEDTAVYLCAAGDSGGSNYKLTFGKGTLLTVTPNIQNPEPAVYQLKDPR | |
| | SQDSTLCLFTDFDSQINVPKTMESGTFITDKTVLDMKAMDSKSNGAIAWSNQ | |
| | TSFTCQDIFKETNATYPSSDVPCDATLTEKSFETDMNLNFQNLSVMGLRILL | |
| | LKVAGFNLLMTLRLWSS | |
| CDR1ß | NSHNY | 2681 |
| CDR2ß | SYGAGN | 2682 |
| CDR3ß | ASASWGGYAEQF | 2683 |
| Vß w/o signal peptide | AVTQSPRNKVTVTGGNVTLSCRQTNSHNYMYWYRQDTGHGLRLIHYSYGAGN | 2684 |
| (SignalP) | LQIGDVPDGYKATRTTQEDFFLLLELASPSQTSLYFCASASWGGYAEQFFGP | |
| | GTRLTVL | |
| Vß w/o signal peptide | EAAVTQSPRNKVTVTGGNVTLSCRQTNSHNYMYWYRQDTGHGLRLIHYSYGA | 2685 |
| (IMGT) | GNLQIGDVPDGYKATRTTQEDFFLLLELASPSQTSLYFCASASWGGYAEQFF | |
| | GPGTRLTVL | |
| Vß | MXSRLFLVLSLLCTKHMEAAVTQSPRNKVTVTGGNVTLSCRQTNSHNYMYWY | 2686 |
| | RQDTGHGLRLIHYSYGAGNLQIGDVPDGYKATRTTQEDFFLLLELASPSQTS | 2687 |
| | LYFCASASWGGYAEQFFGPGTRLTVL | |
| | (X = any amino acid) | |
| ß chain w/WT signal | MGSRLFLVLSLLCTKHMEAAVTQSPRNKVTVTGGNVTLSCRQTNSHNYMYWY | 2688 |
| peptide, Cß | RQDTGHGLRLIHYSYGAGNLQIGDVPDGYKATRTTQEDFFLLLELASPSQTS | |
| (substituted) | LYFCASASWGGYAEQFFGPGTRLTVLEDLRNVTPPKVSLFEPSKAEIANKQK | |
| | ATLVCLARGFFPDHVELSWWVNGKEVHSGVSTDPQAYKESNYSYCLSSRLRV | |
| | SATFWHNPRNHFRCQVQFHGLSEEDKWPEGSPKPVTQNISAEAWGRADCGIT | |
| | SASYHQGVLSATILYEILLGKATLYAVLVSGLVLMAMVKKKNS | |
| ß chain w/alternative | MASRLFLVLSLLCTKHMEAAVTQSPRNKVTVTGGNVTLSCRQTNSHNYMYWY | 2689 |
| signal peptide, Cß | RQDTGHGLRLIHYSYGAGNLQIGDVPDGYKATRTTQEDFFLLLELASPSQTS | |
| (substituted) | LYFCASASWGGYAEQFFGPGTRLTVLEDLRNVTPPKVSLFEPSKAEIANKQK | |
| | ATLVCLARGFFPDHVELSWWVNGKEVHSGVSTDPQAYKESNYSYCLSSRLRV | |
| | SATFWHNPRNHFRCQVQFHGLSEEDKWPEGSPKPVTQNISAEAWGRADCGIT | |
| | SASYHQGVLSATILYEILLGKATLYAVLVSGLVLMAMVKKKNS | |
| ß chain w/alternative | MHSRLFLVLSLLCTKHMEAAVTQSPRNKVTVTGGNVTLSCRQTNSHNYMYWY | 2690 |
| signal peptide, Cß | RQDTGHGLRLIHYSYGAGNLQIGDVPDGYKATRTTQEDFFLLLELASPSQTS | |
| (substituted) | LYFCASASWGGYAEQFFGPGTRLTVLEDLRNVTPPKVSLFEPSKAEIANKQK | |
| | ATLVCLARGFFPDHVELSWWVNGKEVHSGVSTDPQAYKESNYSYCLSSRLRV | |
| | SATFWHNPRNHFRCQVQFHGLSEEDKWPEGSPKPVTQNISAEAWGRADCGIT | |
| | SASYHQGVLSATILYEILLGKATLYAVLVSGLVLMAMVKKKNS | |

In some embodiments, TCR069 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR069 interacts with the neoantigen in the context of HLA-A11, as described in International Publication No. WO 2016/085904, incorporated herein by reference in its entirety.

TABLE 6BR
Amino acid sequences of TCR070.

| | | SEQ |
|---|---|---|
| | | ID |
| Description | Sequence | NO: |
| CDR1α | TTMRS | 1691 |
| CDR2α | LASGT | 1692 |
| CDR3α | AADSSNTGYQNFY | 1693 |
| Vα w/o signal peptide | DQVEQSPSALSLHEGTDSALRCNFTTTMRSVQWFRQNSRGSLISLFYLASGT | 1694 |
| (SignalP) | KENGRLKSAFDSKERRYSTLHIRDAQLEDSGTYFCAADSSNTGYQNFYFGKG | |
| | TSLTVIP | |
| Vα w/o signal peptide | GDQVEQSPSALSLHEGTDSALRCNFTTTMRSVQWFRQNSRGSLISLFYLASG | 1695 |
| (IMGT) | TKENGRLKSAFDSKERRYSTLHIRDAQLEDSGTYFCAADSSNTGYQNFYFGK | |
| | GTSLTVIP | |
| Vα | MXRNLGAVLGILWVQICWVRGDQVEQSPSALSLHEGTDSALRCNFTTTMRSV | 1696 |
| | QWFRQNSRGSLISLFYLASGTKENGRLKSAFDSKERRYSTLHIRDAQLEDSG | 1697 |
| | TYFCAADSSNTGYQNFYFGKGTSLTVIP | |
| | (X = any amino acid) | |
| α chain w/WT signal | MQRNLGAVLGILWVQICWVRGDQVEQSPSALSLHEGTDSALRCNFTTTMRSV | 1698 |
| peptide, Cα | QWFRQNSRGSLISLFYLASGTKENGRLKSAFDSKERRYSTLHIRDAQLEDSG | |
| (substituted) | TYFCAADSSNTGYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGENL | |
| | LMTLRLWSS | |
| α chain w/alternative | MARNLGAVLGILWVQICWVRGDQVEQSPSALSLHEGTDSALRCNFTTTMRSV | 1699 |
| signal peptide, Cα | QWFRQNSRGSLISLFYLASGTKENGRLKSAFDSKERRYSTLHIRDAQLEDSG | |
| (substituted) | TYFCAADSSNTGYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGENL | |
| | LMTLRLWSS | |
| α chain w/alternative | MHRNLGAVLGILWVQICWVRGDQVEQSPSALSLHEGTDSALRCNFTTTMRSV | 1700 |
| signal peptide, Cα | QWFRQNSRGSLISLFYLASGTKENGRLKSAFDSKERRYSTLHIRDAQLEDSG | |
| (substituted) | TYFCAADSSNTGYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGENL | |
| | LMTLRLWSS | |
| CDR1ß | SGHLS | 2691 |
| CDR2ß | HYDKME | 2692 |
| CDR3ß | ASSLTDPLDSDYT | 2693 |
| Vß w/o signal peptide | NSGVVQSPRYIIKGKGERSILKCIPISGHLSVAWYQQTQGQELKEFIQHYDK | 2694 |
| (SignalP) | MERDKGNLPSRFSVQQFDDYHSEMNMSALELEDSAVYFCASSLTDPLDSDYT | |
| | FGSGTRLLVI | |
| Vß w/o signal peptide | SGVVQSPRYIIKGKGERSILKCIPISGHLSVAWYQQTQGQELKFFIQHYDKM | 2695 |
| (IMGT) | ERDKGNLPSRFSVQQFDDYHSEMNMSALELEDSAVYFCASSLTDPLDSDYTF | |
| | GSGTRLLVI | |
| Vß | MXNTAFPDPAWNTTLLSWVALFLLGTSSANSGVVQSPRYIIKGKGERSILKC | 2696 |
| | IPISGHLSVAWYQQTQGQELKFFIQHYDKMERDKGNLPSRFSVQQFDDYHSE | 2697 |
| | MNMSALELEDSAVYFCASSLTDPLDSDYTFGSGTRLLVI | |
| | (X = any amino acid) | |
| ß chain w/WT signal | MSNTAFPDPAWNTTLLSWVALFLLGTSSANSGVVQSPRYIIKGKGERSILKC | 2698 |
| peptide, Cß | IPISGHLSVAWYQQTQGQELKFFIQHYDKMERDKGNLPSRFSVQQFDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |
| ß chain w/alternative | MANTAFPDPAWNTTLLSWVALFLLGTSSANSGVVQSPRYIIKGKGERSILKC | 2699 |
| signal peptide, Cß | IPISGHLSVAWYQQTQGQELKFFIQHYDKMERDKGNLPSRFSVQQFDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |
| ß chain w/alternative | MHNTAFPDPAWNTTLLSWVALFLLGTSSANSGVVQSPRYIIKGKGERSILKC | 2700 |
| signal peptide, Cß | IPISGHLSVAWYQQTQGQELKFFIQHYDKMERDKGNLPSRFSVQQFDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |

In some embodiments, TCR070 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR070 interacts with the neoantigen in the context of HLA-A11, as described in International Publication No. WO 2016/085904, incorporated herein by reference in its entirety.

TABLE 6BS
Amino acid sequences of TCR071.

| | | SEQ |
|---|---|---|
| | | ID |
| Description | Sequence | NO: |
| CDR1α | TTMRS | 1701 |
| CDR2α | LASGT | 1702 |
| CDR3α | AADSSNTZYQNFY | 1703 |
| | (Z = alanine, arginine, asparagine, aspartic acid, cysteine, | |
| | glutamic acid, glutamine, histidine, isoleucine, leucine, lysine, | |
| | methionine, phenylalanine,proline, serine, threonine, tryptophan, | |
| | tyrosine, or valine) | |
| Vα w/o signal peptide | DQVEQSPSALSLHEGTDSALRCNFTTTMRSVQWFRQNSRGSLISLFYLASGT | 1704 |
| (SignalP) | KENGRLKSAFDSKERRYSTLHIRDAQLEDSGTYFCAADSSNTZYQNFYFGKG | |
| | TSLTVIP | |
| | (Z = alanine, arginine, asparagine, aspartic acid, cysteine, | |
| | glutamic acid, glutamine, histidine, isoleucine, leucine, lysine, | |
| | methionine, phenylalanine, proline, serine, threonine, tryptophan, | |
| | tyrosine, or valine) | |
| Vα w/o signal peptide | GDQVEQSPSALSLHEGTDSALRCNFTTTMRSVQWFRQNSRGSLISLFYLASG | 1705 |
| (IMGT) | TKENGRLKSAFDSKERRYSTLHIRDAQLEDSGTYFCAADSSNTZYQNFYFGK | |
| | GTSLTVIP | |
| | (Z = alanine, arginine, asparagine, aspartic acid, cysteine, | |
| | glutamic acid, glutamine, histidine, isoleucine, leucine, lysine, | |
| | methionine, phenylalanine, proline, serine, threonine, tryptophan, | |
| | tyrosine, or valine) | |
| Vα | MXRNLGAVLGILWVQICWVRGDQVEQSPSALSLHEGTDSALRCNFTTTMRSV | 1706 |
| | QWFRQNSRGSLISLFYLASGTKENGRLKSAFDSKERRYSTLHIRDAQLEDSG | |
| | TYFCAADSSNTZYQNFYFGKGTSLTVIP | |
| | (X = any amino acid) | |
| | (Z = alanine, arginine, asparagine, aspartic acid, cysteine, | 1707 |
| | glutamic acid, glutamine, histidine, isoleucine, leucine, lysine, | |
| | methionine, phenylalanine, proline, serine, threonine, tryptophan, | |
| | tyrosine, or valine) | |
| α chain w/WT signal | MQRNLGAVLGILWVQICWVRGDQVEQSPSALSLHEGTDSALRCNFTTTMRSV | 1708 |
| peptide, Cα | QWFRQNSRGSLISLFYLASGTKENGRLKSAFDSKERRYSTLHIRDAQLEDSG | |
| (substituted) | TYFCAADSSNTZYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGENL | |
| | LMTLRLWSS | |
| | (Z = alanine, arginine, asparagine, aspartic acid, cysteine, | |
| | glutamic acid, glutamine, histidine, isoleucine, leucine, lysine, | |
| | methionine, phenylalanine, proline, serine, threonine, tryptophan, | |
| | tyrosine, or valine) | |
| α chain w/alternative | MARNLGAVLGILWVQICWVRGDQVEQSPSALSLHEGTDSALRCNFTTTMRSV | 1709 |
| signal peptide, Cα | QWFRQNSRGSLISLFYLASGTKENGRLKSAFDSKERRYSTLHIRDAQLEDSG | |
| (substituted) | TYFCAADSSNTZYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGENL | |
| | LMTLRLWSS | |
| | (Z = alanine, arginine, asparagine, aspartic acid, cysteine, | |
| | glutamic acid, glutamine, histidine, isoleucine, leucine, lysine, | |
| | methionine, phenylalanine, proline, serine, threonine, tryptophan, | |
| | tyrosine, or valine) | |
| α chain w/alternative | MHRNLGAVLGILWVQICWVRGDQVEQSPSALSLHEGTDSALRCNFTTTMRSV | 1710 |
| signal peptide, Cα | QWFRQNSRGSLISLFYLASGTKENGRLKSAFDSKERRYSTLHIRDAQLEDSG | |
| (substituted) | TYFCAADSSNTZYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGENL | |
| | LMTLRLWSS | |
| | (Z = alanine, arginine, asparagine, aspartic acid, cysteine, | |
| | glutamic acid, glutamine, histidine, isoleucine, leucine, lysine, | |
| | methionine, phenylalanine, proline, serine, threonine, tryptophan, | |
| | tyrosine, or valine) | |
| CDR1ß | SGHLS | 2701 |
| CDR2ß | HYDKME | 2702 |
| CDR3ß | CASSLTDPLDSDYTF | 2703 |
| Vß w/o signal peptide | NSGVVQSPRYIIKGKGERSILKCIPISGHLSVAWYQQTQGQELKFFIQHYDK | 2704 |
| (SignalP) | MERDKGNLPSRFSVQQFDDYHSEMNMSALELEDSAVYFCASSLTDPLDSDYT | |
| | FGSGTRLLVI | |
| Vß w/o signal peptide | SGVVQSPRYIIKGKGERSILKCIPISGHLSVAWYQQTQGQELKFFIQHYDKM | 2705 |
| (IMGT) | ERDKGNLPSRFSVQQFDDYHSEMNMSALELEDSAVYFCASSLTDPLDSDYTF | |
| | GSGTRLLVI | |
| Vß | MXNTAFPDPAWNTTLLSWVALFLLGTSSANSGVVQSPRYIIKGKGERSILKC | 2706 |
| | IPISGHLSVAWYQQTQGQELKFFIQHYDKMERDKGNLPSRFSVQQFDDYHSE | |
| | MNMSALELEDSAVYFCASSLTDPLDSDYTFGSGTRLLVI | 2707 |
| | (X = any amino acid) | |
| ß chain w/WT signal | MSNTAFPDPAWNTTLLSWVALFLLGTSSANSGVVQSPRYIIKGKGERSILKC | 2708 |
| peptide, Cß | IPISGHLSVAWYQQTQGQELKFFIQHYDKMERDKGNLPSRFSVQQFDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |
| ß chain w/alternative | MANTAFPDPAWNTTLLSWVALFLLGTSSANSGVVQSPRYIIKGKGERSILKC | 2709 |
| signal peptide, Cß | IPISGHLSVAWYQQTQGQELKFFIQHYDKMERDKGNLPSRFSVQQEDDYHSE | 2710 |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |

In some embodiments, TCR071 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR071 interacts with the neoantigen in the context of HLA-A11, as described in International Publication No. WO 2016/085904, incorporated herein by reference in its entirety.

TABLE 6BT
Amino acid sequences of TCR072.

| | | SEQ |
|---|---|---|
| | | ID |
| Description | Sequence | NO: |
| CDR1α | DRGSQS | 1711 |
| CDR2α | IYSNGD | 1712 |
| CDR3α | AVEGAGSYQLT | 1713 |
| Vα w/o signal peptide | QQKEVEQNSGPLSVPEGAIASLNCTYSDRGSQSFFWYRQYSGKSPELIMFIY | 1714 |
| (SignalP) | SNGDKEDGRFTAQLNKASQYVSLLIRDSQPSDSATYLCAVEGAGSYQLTFGK | |
| | GTKLSVIP | |
| Vα w/o signal peptide | QKEVEQNSGPLSVPEGAIASLNCTYSDRGSQSFFWYRQYSGKSPELIMFIYS | 1715 |
| (IMGT) | NGDKEDGRFTAQLNKASQYVSLLIRDSQPSDSATYLCAVEGAGSYQLTFGKG | |
| | TKLSVIP | |
| Vα | MXSLRVLLVILWLQLSWVWSQQKEVEQNSGPLSVPEGAIASLNCTYSDRGSQ | 1716 |
| | SFFWYRQYSGKSPELIMFIYSNGDKEDGRFTAQLNKASQYVSLLIRDSQPSD | 1717 |
| | SATYLCAVEGAGSYQLTFGKGTKLSVIP | |
| | (X = any amino acid) | |
| α chain w/WT signal | MKSLRVLLVILWLQLSWVWSQQKEVEQNSGPLSVPEGAIASLNCTYSDRGSQ | 1718 |
| peptide, Cα | SFFWYRQYSGKSPELIMFIYSNGDKEDGRFTAQLNKASQYVSLLIRDSQPSD | |
| (substituted) | SATYLCAVEGAGSYQLTFGKGTKLSVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MASLRVLLVILWLQLSWVWSQQKEVEQNSGPLSVPEGAIASLNCTYSDRGSQ | 1719 |
| signal peptide, Cα | SFFWYRQYSGKSPELIMFIYSNGDKEDGRFTAQLNKASQYVSLLIRDSQPSD | |
| (substituted) | SATYLCAVEGAGSYQLTFGKGTKLSVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MHSLRVLLVILWLQLSWVWSQQKEVEQNSGPLSVPEGAIASLNCTYSDRGSQ | 1720 |
| signal peptide, Cα | SFFWYRQYSGKSPELIMFIYSNGDKEDGRFTAQLNKASQYVSLLIRDSQPSD | |
| (substituted) | SATYLCAVEGAGSYQLTFGKGTKLSVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| CDR1ß | DFQATT | 2711 |
| CDR2ß | SNEGSKA | 2712 |
| CDR3ß | SASNRLAVNEQF | 2713 |
| Vß w/o signal peptide | GSGLGAVVSQHPSWVICKSGTSVKIECRSLDFQATTMFWYRQFPKQSLMLMA | 2714 |
| (SignalP) | TSNEGSKATYEQGVEKDKFLINHASLTLSTLTVTSAHPEDSSFYICSASNRL | |
| | AVNEQFFGPGTRLTVL | |
| Vß w/o signal peptide | GAVVSQHPSWVICKSGTSVKIECRSLDFQATTMFWYRQFPKQSLMLMATSNE | 2715 |
| (IMGT) | GSKATYEQGVEKDKFLINHASLTLSTLTVTSAHPEDSSFYICSASNRLAVNE | |
| | QFFGPGTRLTVL | |
| Vß | MXLLLLLLGPGISLLLPGSLAGSGLGAVVSQHPSWVICKSGTSVKIECRSLD | 2716 |
| | FQATTMFWYRQFPKQSLMLMATSNEGSKATYEQGVEKDKFLINHASLTLSTL | |
| | TVTSAHPEDSSFYICSASNRLAVNEQFFGPGTRLTVL | 2717 |
| | (X = any amino acid) | |
| ß chain w/WT signal | MLLLLLLLGPGISLLLPGSLAGSGLGAVVSQHPSWVICKSGTSVKIECRSLD | 2718 |
| peptide, Cß | FQATTMFWYRQFPKQSLMLMATSNEGSKATYEQGVEKDKFLINHASLTLSTL | |
| (substituted) | TVTSAHPEDSSFYICSASNRLAVNEQFFGPGTRLTVLEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| ß chain w/alternative | MALLLLLLGPGISLLLPGSLAGSGLGAVVSQHPSWVICKSGTSVKIECRSLD | 2719 |
| signal peptide, Cß | FQATTMFWYRQFPKQSLMLMATSNEGSKATYEQGVEKDKFLINHASLTLSTL | |
| (substituted) | TVTSAHPEDSSFYICSASNRLAVNEQFFGPGTRLTVLEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHERCQVQFHGLSEEDKWPEGSPKPVTQNISA | |
| | EAWGRADCGITSASYQQGVLSATILYEILLGKATLYAVLVSTLVVMAMVKRK | |
| | NS | |
| ß chain w/alternative | MHLLLLLLGPGISLLLPGSLAGSGLGAVVSQHPSWVICKSGTSVKIECRSLD | 2720 |
| signal peptide, Cß | FQATTMFWYRQFPKQSLMLMATSNEGSKATYEQGVEKDKFLINHASLTLSTL | |
| (substituted) | TVTSAHPEDSSFYICSASNRLAVNEQFFGPGTRLTVLEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |

In some embodiments, TCR072 interacts with and/or is specific for KRAS. In some embodiments, the peptide is from a neoantigen of KRAS. In some embodiments, the neoantigen has the amino acid change G12R relative to the wild type KRAS sequence. In some embodiments, TCR072 interacts with the neoantigen in the context of HLA-DQA1*05:05:HLA-DQB1*03:01 heterodimer as described in International Publication No. WO 2020/154275, incorporated herein by reference in its entirety.

TABLE 6BU
Amino acid sequences of TCR073.

| | | SEQ |
|---|---|---|
| | | ID |
| Description | Sequence | NO: |
| CDR1α | NSMEDY | 1721 |
| CDR2α | ISISSIKDK | 1722 |
| CDR3α | AASGNTGTASKLT | 1723 |
| Vα w/o signal peptide | QQKNDDQQVKQNSPSLSVQEGRISILNCDYTNSMFDYFLWYKKYPAEGPTEL | 1724 |
| (SignalP) | ISISSIKDKNEDGRFTVFLNKSAKHLSLHIVPSQPGDSAVYFCAASGNTGTA | |
| | SKLTFGTGTRLQVTL | |
| Vα w/o signal peptide | DQQVKQNSPSLSVQEGRISILNCDYTNSMFDYFLWYKKYPAEGPTFLISISS | 1725 |
| (IMGT) | IKDKNEDGRFTVFLNKSAKHLSLHIVPSQPGDSAVYFCAASGNTGTASKLTF | |
| | GTGTRLQVTL | |
| Vα | MXMLLGASVLILWLQPDWVNSQQKNDDQQVKQNSPSLSVQEGRISILNCDYT | 1726 |
| | NSMFDYFLWYKKYPAEGPTFLISISSIKDKNEDGRFTVFLNKSAKHLSLHIV | 1727 |
| | PSQPGDSAVYFCAASGNTGTASKLTFGTGTRLQVTL | |
| | (X = any amino acid) | |
| α chain w/WT signal | MAMLLGASVLILWLQPDWVNSQQKNDDQQVKQNSPSLSVQEGRISILNCDYT | 1728 |
| peptide, Cα | NSMFDYFLWYKKYPAEGPTFLISISSIKDKNEDGRFTVFLNKSAKHLSLHIV | |
| (substituted) | PSQPGDSAVYFCAASGNTGTASKLTFGTGTRLQVTLNIQNPEPAVYQLKDPR | |
| | SQDSTLCLFTDFDSQINVPKTMESGTFITDKCVLDMKAMDSKSNGAIAWSNQ | |
| | TSFTCQDIFKETNATYPSSDVPCDATLTEKSFETDMNLNFQNLLVIVLRILL | |
| | LKVAGENLLMTLRLWSS | |
| α chain w/alternative | MHMLLGASVLILWLQPDWVNSQQKNDDQQVKQNSPSLSVQEGRISILNCDYT | 1729 |
| signal peptide, Cα | NSMFDYFLWYKKYPAEGPTFLISISSIKDKNEDGRFTVFLNKSAKHLSLHIV | 1730 |
| (substituted) | PSQPGDSAVYFCAASGNTGTASKLTFGTGTRLQVTLNIQNPEPAVYQLKDPR | |
| | SQDSTLCLFTDFDSQINVPKTMESGTFITDKCVLDMKAMDSKSNGAIAWSNQ | |
| | TSFTCQDIFKETNATYPSSDVPCDATLTEKSFETDMNLNFQNLLVIVLRILL | |
| | LKVAGFNLLMTLRLWSS | |
| CDR1ß | LNHDA | 2721 |
| CDR2ß | SQIVND | 2722 |
| CDR3ß | ASSNQLVTGGYGYT | 2723 |
| Vß w/o signal peptide | GITQSPKYLFRKEGQNVTLSCEQNLNHDAMYWYRQDPGQGLRLIYYSQIVND | 2724 |
| (SignalP) | FQKGDIAEGYSVSREKKESFPLTVTSAQKNPTAFYLCASSNQLVTGGYGYTF | |
| | GSGTRLTVV | |
| Vß w/o signal peptide | DGGITQSPKYLFRKEGQNVTLSCEQNLNHDAMYWYRQDPGQGLRLIYYSQIV | 2725 |
| (IMGT) | NDFQKGDIAEGYSVSREKKESFPLTVTSAQKNPTAFYLCASSNQLVTGGYGY | |
| | TFGSGTRLTVV | |
| Vß | MXNQVLCCVVLCFLGANTVDGGITQSPKYLFRKEGQNVTLSCEQNLNHDAMY | 2726 |
| | WYRQDPGQGLRLIYYSQIVNDFQKGDIAEGYSVSREKKESFPLTVTSAQKNP | 2727 |
| | TAFYLCASSNQLVTGGYGYTFGSGTRLTVV | |
| | (X = any amino acid) | |
| ß chain w/WT signal | MSNQVLCCVVLCFLGANTVDGGITQSPKYLFRKEGQNVTLSCEQNLNHDAMY | 2728 |
| peptide, Cß | WYRQDPGQGLRLIYYSQIVNDFQKGDIAEGYSVSREKKESFPLTVTSAQKNP | |
| (substituted) | TAFYLCASSNQLVTGGYGYTFGSGTRLTVVEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MANQVLCCVVLCFLGANTVDGGITQSPKYLFRKEGQNVTLSCEQNLNHDAMY | 2729 |
| signal peptide, Cß | WYRQDPGQGLRLIYYSQIVNDFQKGDIAEGYSVSREKKESFPLTVTSAQKNP | |
| (substituted) | TAFYLCASSNQLVTGGYGYTFGSGTRLTVVEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MHNQVLCCVVLCFLGANTVDGGITQSPKYLFRKEGQNVTLSCEQNLNHDAMY | 2730 |
| signal peptide, Cß | WYRQDPGQGLRLIYYSQIVNDFQKGDIAEGYSVSREKKESFPLTVTSAQKNP | |
| (substituted) | TAFYLCASSNQLVTGGYGYTFGSGTRLTVVEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR073 interacts with and/or is specific for KRAS. In some embodiments, the peptide is from a neoantigen of KRAS. In some embodiments, the neoantigen has the amino acid change G12R relative to the wild type KRAS sequence. In some embodiments, TCR073 interacts with the neoantigen in the context of HLA-DRB5*01:HLA-DRA*01:01 heterodimer as described in International Publication No. WO 2020/154275, incorporated herein by reference in its entirety.

TABLE 6BV
Amino acid sequences of TCR074.

| | | SEQ |
|---|---|---|
| | | ID |
| Description | Sequence | NO: |
| CDR1α | TIYSNAF | 1731 |
| CDR2α | SSTDNKR | 1732 |
| CDR3α | ALSEGGNYKYV | 1733 |
| Vα w/o signal peptide | DGDSVTQKEGLVTLTEGLPVMLNCTYQTIYSNAFLFWYVHYLNESPRLLLKS | 1734 |
| (SignalP) | STDNKRTEHQGFHATLHKSSSSFHLQKSSAQLSDSALYYCALSEGGNYKYVF | |
| | GAGTRLKVIA | |
| Vα w/o signal peptide | GDSVTQKEGLVTLTEGLPVMLNCTYQTIYSNAFLFWYVHYLNESPRLLLKSS | 1735 |
| (IMGT) | TDNKRTEHQGFHATLHKSSSSFHLQKSSAQLSDSALYYCALSEGGNYKYVFG | |
| | AGTRLKVIA | |
| Vα | MXPGTCSVLVLLLMLRRSNGDGDSVTQKEGLVTLTEGLPVMLNCTYQTIYSN | 1736 |
| | AFLFWYVHYLNESPRLLLKSSTDNKRTEHQGFHATLHKSSSSFHLQKSSAQL | 1737 |
| | SDSALYYCALSEGGNYKYVFGAGTRLKVIA | |
| | (X = any amino acid) | |
| α chain w/WT signal | MRPGTCSVLVLLLMLRRSNGDGDSVTQKEGLVTLTEGLPVMLNCTYQTIYSN | 1738 |
| peptide, Cα | AFLFWYVHYLNESPRLLLKSSTDNKRTEHQGFHATLHKSSSSFHLQKSSAQL | |
| (substituted) | SDSALYYCALSEGGNYKYVFGAGTRLKVIANIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain w/alternative | MAPGTCSVLVLLLMLRRSNGDGDSVTQKEGLVTLTEGLPVMLNCTYQTIYSN | 1739 |
| signal peptide, Cα | AFLFWYVHYLNESPRLLLKSSTDNKRTEHQGFHATLHKSSSSFHLQKSSAQL | |
| (substituted) | SDSALYYCALSEGGNYKYVFGAGTRLKVIANIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain w/alternative | MHPGTCSVLVLLLMLRRSNGDGDSVTQKEGLVTLTEGLPVMLNCTYQTIYSN | 1740 |
| signal peptide, Cα | AFLFWYVHYLNESPRLLLKSSTDNKRTEHQGFHATLHKSSSSFHLQKSSAQL | |
| (substituted) | SDSALYYCALSEGGNYKYVFGAGTRLKVIANIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGE | |
| | NLLMTLRLWSS | |
| CDR1ß | NSQYPW | 2731 |
| CDR2ß | LRSPGD | 2732 |
| CDR3ß | TCSARHSAETLY | 2733 |
| Vß w/o signal peptide | DPTVTLLEQNPRWRLVPRGQAVNLRCILKNSQYPWMSWYQQDLQKQLQWLFT | 2734 |
| (SignalP) | LRSPGDKEVKSLPGADYLATRVTDTELRLQVANMSQGRTLYCTCSARHSAET | |
| | LYFGSGTRLTVL | |
| Vß w/o signal peptide | VTLLEQNPRWRLVPRGQAVNLRCILKNSQYPWMSWYQQDLQKQLQWLFTLRS | 2735 |
| (IMGT) | PGDKEVKSLPGADYLATRVTDTELRLQVANMSQGRTLYCTCSARHSAETLYF | |
| | GSGTRLTVL | |
| Vß | MXQFCILCLCVLMASVATDPTVTLLEQNPRWRLVPRGQAVNLRCILKNSQYP | 2736 |
| | WMSWYQQDLQKQLQWLFTLRSPGDKEVKSLPGADYLATRVTDTELRLQVANM | 2737 |
| | SQGRTLYCTCSARHSAETLYFGSGTRLTVL | |
| | (X = any amino acid) | |
| ß chain w/WT signal | MWQFCILCLCVLMASVATDPTVTLLEQNPRWRLVPRGQAVNLRCILKNSQYP | 2738 |
| peptide, Cß | WMSWYQQDLQKQLQWLFTLRSPGDKEVKSLPGADYLATRVTDTELRLQVANM | |
| (substituted) | SQGRTLYCTCSARHSAETLYFGSGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MAQFCILCLCVLMASVATDPTVTLLEQNPRWRLVPRGQAVNLRCILKNSQYP | 2739 |
| signal peptide, Cß | WMSWYQQDLQKQLQWLFTLRSPGDKEVKSLPGADYLATRVTDTELRLQVANM | |
| (substituted) | SQGRTLYCTCSARHSAETLYFGSGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MHQFCILCLCVLMASVATDPTVTLLEQNPRWRLVPRGQAVNLRCILKNSQYP | 2740 |
| signal peptide, Cß | WMSWYQQDLQKQLQWLFTLRSPGDKEVKSLPGADYLATRVTDTELRLQVANM | |
| (substituted) | SQGRTLYCTCSARHSAETLYFGSGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR074 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR074 interacts with the neoantigen in the context of HLA-A3 heterodimer as described in International Publication No. WO 2020/086827, incorporated herein by reference in its entirety.

TABLE 6BW
Amino acid sequences of TCR075.

| | | SEQ |
|---|---|---|
| | | ID |
| Description | Sequence | NO: |
| CDR1α | TSDPSYG | 1741 |
| CDR2α | QGSYDQQN | 1742 |
| CDR3α | AMRGASQGGSEKLV | 1743 |
| Vα w/o signal peptide | QKITQTQPGMFVQEKEAVTLDCTYDTSDPSYGLFWYKQPSSGEMIFLIYQGS | 1744 |
| (SignalP) | YDQQNATEGRYSLNFQKARKSANLVISASQLGDSAMYFCAMRGASQGGSEKL | |
| | VFGKGTKLTVNP | |
| Vα w/o signal peptide | AQKITQTQPGMFVQEKEAVTLDCTYDTSDPSYGLFWYKQPSSGEMIFLIYQG | 1745 |
| (IMGT) | SYDQQNATEGRYSLNFQKARKSANLVISASQLGDSAMYFCAMRGASQGGSEK | |
| | LVFGKGTKLTVNP | |
| Vα | MXLSSLLKVVTASLWLGPGIAQKITQTQPGMFVQEKEAVTLDCTYDTSDPSY | 1746 |
| | GLFWYKQPSSGEMIFLIYQGSYDQQNATEGRYSLNFQKARKSANLVISASQL | 1747 |
| | GDSAMYFCAMRGASQGGSEKLVFGKGTKLTVNP | |
| | (X = any amino acid) | |
| α chain w/WT signal | MSLSSLLKVVTASLWLGPGIAQKITQTQPGMFVQEKEAVTLDCTYDTSDPSY | 1748 |
| peptide, Cα | GLFWYKQPSSGEMIFLIYQGSYDQQNATEGRYSLNFQKARKSANLVISASQL | |
| (substituted) | GDSAMYFCAMRGASQGGSEKLVFGKGTKLTVNPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MALSSLLKVVTASLWLGPGIAQKITQTQPGMFVQEKEAVTLDCTYDTSDPSY | 1749 |
| signal peptide, Cα | GLFWYKQPSSGEMIFLIYQGSYDQQNATEGRYSLNFQKARKSANLVISASQL | |
| (substituted) | GDSAMYFCAMRGASQGGSEKLVFGKGTKLTVNPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MHLSSLLKVVTASLWLGPGIAQKITQTQPGMFVQEKEAVTLDCTYDTSDPSY | 1750 |
| signal peptide, Cα | GLFWYKQPSSGEMIFLIYQGSYDQQNATEGRYSLNFQKARKSANLVISASQL | |
| (substituted) | GDSAMYFCAMRGASQGGSEKLVFGKGTKLTVNPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| CDR1ß | SGHRS | 2741 |
| CDR2ß | YFSETQ | 2742 |
| CDR3ß | ASSLTSGGFDEQF | 2743 |
| Vß w/o signal peptide | GVTQTPRYLIKTRGQQVTLSCSPISGHRSVSWYQQTPGQGLQFLFEYFSETQ | 2744 |
| (SignalP) | RNKGNFPGRFSGRQFSNSRSEMNVSTLELGDSALYLCASSLTSGGFDEQFFG | |
| | PGTRLTVL | |
| Vß w/o signal peptide | KAGVTQTPRYLIKTRGQQVTLSCSPISGHRSVSWYQQTPGQGLQFLFEYFSE | 2745 |
| (IMGT) | TQRNKGNFPGRFSGRQFSNSRSEMNVSTLELGDSALYLCASSLTSGGFDEQF | |
| | FGPGTRLTVL | |
| Vß | MXSRLLCWVLLCLLGAGPVKAGVTQTPRYLIKTRGQQVTLSCSPISGHRSVS | 2746 |
| | WYQQTPGQGLQFLFEYFSETQRNKGNFPGRFSGRQFSNSRSEMNVSTLELGD | 2747 |
| | SALYLCASSLTSGGFDEQFFGPGTRLTVL | |
| | (X = any amino acid) | |
| ß chain w/WT signal | MGSRLLCWVLLCLLGAGPVKAGVTQTPRYLIKTRGQQVTLSCSPISGHRSVS | 2748 |
| peptide, Cß | WYQQTPGQGLQFLFEYFSETQRNKGNFPGRESGRQFSNSRSEMNVSTLELGD | |
| (substituted) | SALYLCASSLTSGGFDEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MASRLLCWVLLCLLGAGPVKAGVTQTPRYLIKTRGQQVTLSCSPISGHRSVS | 2749 |
| signal peptide, Cß | WYQQTPGQGLQFLFEYFSETQRNKGNFPGRFSGRQFSNSRSEMNVSTLELGD | |
| (substituted) | SALYLCASSLTSGGFDEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MHSRLLCWVLLCLLGAGPVKAGVTQTPRYLIKTRGQQVTLSCSPISGHRSVS | 2750 |
| signal peptide, Cß | WYQQTPGQGLQFLFEYFSETQRNKGNFPGRFSGRQFSNSRSEMNVSTLELGD | |
| (substituted) | SALYLCASSLTSGGFDEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR075 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR075 interacts with the neoantigen in the context of HLA-A*11:01, as described in International Publication No. WO 2019/112941, incorporated herein by reference in its entirety.

TABLE 6BX
Amino acid sequences of TCR076.

| | | SEQ |
|---|---|---|
| | | ID |
| Description | Sequence | NO: |
| CDR1α | DSASNY | 1751 |
| CDR2α | IRSNVGE | 1752 |
| CDR3α | AASTGGGNKLT | 1753 |
| Vα w/o signal peptide | ENVEQHPSTLSVQEGDSAVIKCTYSDSASNYFPWYKQELGKGPQLIIDIRSN | 1754 |
| (SignalP) | VGEKKDQRIAVTLNKTAKHFSLHITETQPEDSAVYFCAASTGGGNKLTFGTG | |
| | TQLKVEL | |
| Vα w/o signal peptide | GENVEQHPSTLSVQEGDSAVIKCTYSDSASNYFPWYKQELGKGPQLIIDIRS | 1755 |
| (IMGT) | NVGEKKDQRIAVTLNKTAKHFSLHITETQPEDSAVYFCAASTGGGNKLTFGT | |
| | GTQLKVEL | |
| Vα | MXSIRAVFIFLWLQLDLVNGENVEQHPSTLSVQEGDSAVIKCTYSDSASNYF | 1756 |
| | PWYKQELGKGPQLIIDIRSNVGEKKDQRIAVTLNKTAKHFSLHITETQPEDS | |
| | AVYFCAASTGGGNKLTFGTGTQLKVEL | 1757 |
| | (X = any amino acid) | |
| α chain w/WT signal | MTSIRAVFIFLWLQLDLVNGENVEQHPSTLSVQEGDSAVIKCTYSDSASNYE | 1758 |
| peptide, Cα | PWYKQELGKGPQLIIDIRSNVGEKKDQRIAVTLNKTAKHFSLHITETQPEDS | |
| (substituted) | AVYFCAASTGGGNKLTFGTGTQLKVELNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain w/alternative | MASIRAVFIFLWLQLDLVNGENVEQHPSTLSVQEGDSAVIKCTYSDSASNYF | 1759 |
| signal peptide, Cα | PWYKQELGKGPQLIIDIRSNVGEKKDQRIAVTLNKTAKHFSLHITETQPEDS | |
| (substituted) | AVYFCAASTGGGNKLTFGTGTQLKVELNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain w/alternative | MHSIRAVFIFLWLQLDLVNGENVEQHPSTLSVQEGDSAVIKCTYSDSASNYF | 1760 |
| signal peptide, Cα | PWYKQELGKGPQLIIDIRSNVGEKKDQRIAVTLNKTAKHFSLHITETQPEDS | |
| (substituted) | AVYFCAASTGGGNKLTFGTGTQLKVELNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| CDR1ß | DFQATT | 2751 |
| CDR2ß | SNEGSKA | 2752 |
| CDR3ß | SAREGAGGMGTQY | 2753 |
| Vß w/o signal peptide | AVVSQHPSRVICKSGTSVKIECRSLDFQATTMFWYRQFPKQSLMLMATSNEG | 2754 |
| (SignalP) | SKATYEQGVEKDKFLINHASLTLSTLTVTSAHPEDSSFYICSAREGAGGMGT | |
| | QYFGPGTRLLVL | |
| Vß w/o signal peptide | GAVVSQHPSRVICKSGTSVKIECRSLDFQATTMFWYRQFPKQSLMLMATSNE | 2755 |
| (IMGT) | GSKATYEQGVEKDKFLINHASLTLSTLTVTSAHPEDSSFYICSAREGAGGMG | |
| | TQYFGPGTRLLVL | |
| Vß | MXLLLLLLGPAGSGLGAVVSQHPSRVICKSGTSVKIECRSLDFQATTMFWYR | 2756 |
| | QFPKQSLMLMATSNEGSKATYEQGVEKDKFLINHASLTLSTLTVTSAHPEDS | |
| | SFYICSAREGAGGMGTQYFGPGTRLLVL | 2757 |
| | (X = any amino acid) | |
| ß chain w/WT signal | MLLLLLLLGPAGSGLGAVVSQHPSRVICKSGTSVKIECRSLDFQATTMFWYR | 2758 |
| peptide, Cß | QFPKQSLMLMATSNEGSKATYEQGVEKDKFLINHASLTLSTLTVTSAHPEDS | |
| (substituted) | SFYICSAREGAGGMGTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MALLLLLLGPAGSGLGAVVSQHPSRVICKSGTSVKIECRSLDFQATTMFWYR | 2759 |
| signal peptide, Cß | QFPKQSLMLMATSNEGSKATYEQGVEKDKFLINHASLTLSTLTVTSAHPEDS | |
| (substituted) | SFYICSAREGAGGMGTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MHLLLLLLGPAGSGLGAVVSQHPSRVICKSGTSVKIECRSLDFQATTMFWYR | 2760 |
| signal peptide, Cß | QFPKQSLMLMATSNEGSKATYEQGVEKDKFLINHASLTLSTLTVTSAHPEDS | |
| (substituted) | SFYICSAREGAGGMGTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR076 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR076 interacts with the neoantigen in the context of HLA-DRB1*07:01, as described in International Publication No. WO 2019/060349, incorporated herein by reference in its entirety.

TABLE 6BY
Amino acid sequences of TCR077.

| | | SEQ |
|---|---|---|
| | | ID |
| Description | Sequence | NO: |
| CDR1α | SSVPPY | 1761 |
| CDR2α | YTSAATLV | 1762 |
| CDR3α | AVSEDSNYQLI | 1763 |
| Vα w/o signal peptide | QSVTQLGSHVSVSEGALVLLRCNYSSSVPPYLFWYVQYPNQGLQLLLKYTSA | 1764 |
| (SignalP) | ATLVKGINGFEAEFKKSETSFHLTKPSAHMSDAAEYFCAVSEDSNYQLIWGA | |
| | GTKLIIKP | |
| Vα w/o signal peptide | AQSVTQLGSHVSVSEGALVLLRCNYSSSVPPYLFWYVQYPNQGLQLLLKYTS | 1765 |
| (IMGT) | AATLVKGINGFEAEFKKSETSFHLTKPSAHMSDAAEYFCAVSEDSNYQLIWG | |
| | AGTKLIIKP | |
| Vα | MXLLLVPVLEVIFTLGGTRAQSVTQLGSHVSVSEGALVLLRCNYSSSVPPYL | 1766 |
| | FWYVQYPNQGLQLLLKYTSAATLVKGINGFEAEFKKSETSFHLTKPSAHMSD | 1767 |
| | AAEYFCAVSEDSNYQLIWGAGTKLIIKP | |
| | (X = any amino acid) | |
| α chain w/WT signal | MLLLLVPVLEVIFTLGGTRAQSVTQLGSHVSVSEGALVLLRCNYSSSVPPYL | 1768 |
| peptide, Cα | FWYVQYPNQGLQLLLKYTSAATLVKGINGFEAEFKKSETSFHLTKPSAHMSD | |
| (substituted) | AAEYFCAVSEDSNYQLIWGAGTKLIIKPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MALLLVPVLEVIFTLGGTRAQSVTQLGSHVSVSEGALVLLRCNYSSSVPPYL | 1769 |
| signal peptide, Cα | FWYVQYPNQGLQLLLKYTSAATLVKGINGFEAEFKKSETSFHLTKPSAHMSD | |
| (substituted) | AAEYFCAVSEDSNYQLIWGAGTKLIIKPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/alternative | MHLLLVPVLEVIFTLGGTRAQSVTQLGSHVSVSEGALVLLRCNYSSSVPPYL | 1770 |
| signal peptide, Cα | FWYVQYPNQGLQLLLKYTSAATLVKGINGFEAEFKKSETSFHLTKPSAHMSD | |
| (substituted) | AAEYFCAVSEDSNYQLIWGAGTKLIIKPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| CDR1β | GTSNPN | 2761 |
| CDR2β | SVGIG | 2762 |
| CDR3β | AYSPGLASDTQY | 2763 |
| Vβ w/o signal peptide | QTIHQWPATLVQPVGSPLSLECTVEGTSNPNLYWYRQAAGRGLQLLFYSVGI | 2764 |
| (SignalP) | GQISSEVPQNLSASRPQDRQFILSSKKLLLSDSGFYLCAYSPGLASDTQYFG | |
| | PGTRLTVL | |
| Vβ w/o signal peptide | SQTIHQWPATLVQPVGSPLSLECTVEGTSNPNLYWYRQAAGRGLQLLFYSVG | 2765 |
| (IMGT) | IGQISSEVPQNLSASRPQDRQFILSSKKLLLSDSGFYLCAYSPGLASDTQYF | |
| | GPGTRLTVL | |
| Vβ | MXCSLLALLLGTFFGVRSQTIHQWPATLVQPVGSPLSLECTVEGTSNPNLYW | 2766 |
| | YRQAAGRGLQLLFYSVGIGQISSEVPQNLSASRPQDRQFILSSKKLLLSDSG | 2767 |
| | FYLCAYSPGLASDTQYFGPGTRLTVL | |
| | (X = any amino acid) | |
| β chain w/WT signal | MLCSLLALLLGTFFGVRSQTIHQWPATLVQPVGSPLSLECTVEGTSNPNLYW | 2768 |
| peptide, Cβ | YRQAAGRGLQLLFYSVGIGQISSEVPQNLSASRPQDRQFILSSKKLLLSDSG | |
| (substituted) | FYLCAYSPGLASDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain w/alternative | MACSLLALLLGTFFGVRSQTIHQWPATLVQPVGSPLSLECTVEGTSNPNLYW | 2769 |
| signal peptide, Cβ | YRQAAGRGLQLLFYSVGIGQISSEVPQNLSASRPQDRQFILSSKKLLLSDSG | |
| (substituted) | FYLCAYSPGLASDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain w/alternative | MHCSLLALLLGTFFGVRSQTIHQWPATLVQPVGSPLSLECTVEGTSNPNLYW | 2770 |
| signal peptide, Cβ | YRQAAGRGLQLLFYSVGIGQISSEVPQNLSASRPQDRQFILSSKKLLLSDSG | |
| (substituted) | FYLCAYSPGLASDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR077 interacts with and/or is specific for the epidermal growth factor receptor (EGFR) tumor protein. In some embodiments, the peptide is from a neoantigen of EGFR. In some embodiments, the neoantigen has the amino acid changes E746-A750del relative to the wild type EGFR sequence. In some embodiments, TCR077 interacts with the neoantigen in the context of a heterodimer of HLA-DPA1*02:01 and HLA-DPB1*01:01, as described in International Publication No. WO 2019/213195, incorporated herein by reference in its entirety.

TABLE 6BZ
Amino acid sequences of TCR078.

| | | SEQ |
|---|---|---|
| | | ID |
| Description | Sequence | NO: |
| CDR1α | TSINN | 1771 |
| CDR2α | IRSNERE | 1772 |
| CDR3α | ATDGETSGSRLT | 1773 |
| Vα without signal | QQGEEDPQALSIQEGENATMNCSYKTSINNLQWYRQNSGRGLVHLILIRSNE | 1774 |
| peptide (SignalP) | REKHSGRLRVTLDTSKKSSSLLITASRAADTASYFCATDGETSGSRLTFGEG | |
| | TQLTVNP | |
| Vα without signal | SQQGEEDPQALSIQEGENATMNCSYKTSINNLQWYRQNSGRGLVHLILIRSN | 1775 |
| peptide (IMGT) | EREKHSGRLRVTLDTSKKSSSLLITASRAADTASYFCATDGETSGSRLTFGE | |
| | GTQLTVNP | |
| Vα | MXTLLGVSLVILWLQLARVNSQQGEEDPQALSIQEGENATMNCSYKTSINNL | 1776 |
| | QWYRQNSGRGLVHLILIRSNEREKHSGRLRVTLDTSKKSSSLLITASRAADT | 1777 |
| | ASYFCATDGETSGSRLTFGEGTQLTVNP | |
| | (X = any amino acid) | |
| α chain with WT signal | METLLGVSLVILWLQLARVNSQQGEEDPQALSIQEGENATMNCSYKTSINNL | 1778 |
| peptide, Cα | QWYRQNSGRGLVHLILIRSNEREKHSGRLRVTLDTSKKSSSLLITASRAADT | |
| (substituted) | ASYFCATDGETSGSRLTFGEGTQLTVNPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain with | MATLLGVSLVILWLQLARVNSQQGEEDPQALSIQEGENATMNCSYKTSINNL | 1779 |
| alternative signal | QWYRQNSGRGLVHLILIRSNEREKHSGRLRVTLDTSKKSSSLLITASRAADT | |
| peptide, Cα | ASYFCATDGETSGSRLTFGEGTQLTVNPNIQNPEPAVYQLKDPRSQDSTLCL | |
| (substituted) | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| β | MHTLLGVSLVILWLQLARVNSQQGEEDPQALSIQEGENATMNCSYKTSINNL | 1780 |
| chain with | QWYRQNSGRGLVHLILIRSNEREKHSGRLRVTLDTSKKSSSLLITASRAADT | |
| alternative signal | ASYFCATDGETSGSRLTFGEGTQLTVNPNIQNPEPAVYQLKDPRSQDSTLCL | |
| peptide, Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| CDR1β | DFQATT | 2771 |
| CDR2β | SNEGSKA | 2772 |
| CDR3β | SASRGATGQPQH | 2773 |
| Vβ without signal | AVVSQHPSWVICKSGTSVKIECRSLDFQATTMFWYRQFPKQSLMLMATSNEG | 2774 |
| peptide (SignalP) | SKATYEQGVEKDKFLINHASLTLSTLTVTSAHPEDSSFYICSASRGATGQPQ | |
| | HFGDGTRLSIL | |
| Vβ without signal | GAVVSQHPSWVICKSGTSVKIECRSLDFQATTMFWYRQFPKQSLMLMATSNE | 2775 |
| peptide (IMGT) | GSKATYEQGVEKDKFLINHASLTLSTLTVTSAHPEDSSFYICSASRGATGQP | |
| | QHFGDGTRLSIL | |
| Vβ | MXLLLLLLGPGSGLGAVVSQHPSWVICKSGTSVKIECRSLDFQATTMFWYRQ | 2776 |
| | FPKQSLMLMATSNEGSKATYEQGVEKDKFLINHASLTLSTLTVTSAHPEDSS | 2777 |
| | FYICSASRGATGQPQHFGDGTRLSIL | |
| | (X = any amino acid) | |
| β chain with WT signal | MLLLLLLLGPGSGLGAVVSQHPSWVICKSGTSVKIECRSLDFQATTMFWYRQ | 2778 |
| peptide, Cβ | FPKQSLMLMATSNEGSKATYEQGVEKDKFLINHASLTLSTLTVTSAHPEDSS | |
| (substituted) | FYICSASRGATGQPQHFGDGTRLSILEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with alternative | MALLLLLLGPGSGLGAVVSQHPSWVICKSGTSVKIECRSLDFQATTMFWYRQ | 2779 |
| signal peptide, Cβ | FPKQSLMLMATSNEGSKATYEQGVEKDKFLINHASLTLSTLTVTSAHPEDSS | |
| (substituted) | FYICSASRGATGQPQHFGDGTRLSILEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with alternative | MHLLLLLLGPGSGLGAVVSQHPSWVICKSGTSVKIECRSLDFQATTMFWYRQ | 2789 |
| signal peptide, Cβ | FPKQSLMLMATSNEGSKATYEQGVEKDKFLINHASLTLSTLTVTSAHPEDSS | |
| (substituted) | FYICSASRGATGQPQHFGDGTRLSILEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR078 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR078 interacts with the neoantigen in the context of an HLA-DPA1*01:03 chain and an HLA-DPB1*03:01 chain, as described in International Publication No. WO 2021/173902, incorporated herein by reference in its entirety.

TABLE 6CA
Amino acid sequences of TCR079.

| | | SEQ |
|---|---|---|
| | | ID |
| Description | Sequence | NO: |
| CDR1α | NSASDY | 1781 |
| CDR2α | IRSNMDK | 1782 |
| CDR3α | AERGRGGKLI | 1783 |
| Vα without signal | ESVGLHLPTLSVQEGDNSIINCAYSNSASDYFIWYKQESGKGPQFIIDIRSN | 1784 |
| peptide (SignalP) | MDKRQGQRVTVLLNKTVKHLSLQIAATQPGDSAVYFCAERGRGGKLIFGQGT | |
| | ELSVKP | |
| Vα without signal | GESVGLHLPTLSVQEGDNSIINCAYSNSASDYFIWYKQESGKGPQFIIDIRS | 1785 |
| peptide (IMGT) | NMDKRQGQRVTVLLNKTVKHLSLQIAATQPGDSAVYFCAERGRGGKLIFGQG | |
| | TELSVKP | |
| Vα | MXGIRALFMYLWLQLDWVSRGESVGLHLPTLSVQEGDNSIINCAYSNSASDY | 1786 |
| | FIWYKQESGKGPQFIIDIRSNMDKRQGQRVTVLLNKTVKHLSLQIAATQPGD | 1787 |
| | SAVYFCAERGRGGKLIFGQGTELSVKP | |
| | (X = any amino acid) | |
| α chain with WT signal | MAGIRALFMYLWLQLDWVSRGESVGLHLPTLSVQEGDNSIINCAYSNSASDY | 1788 |
| peptide, Cα | FIWYKQESGKGPQFIIDIRSNMDKRQGQRVTVLLNKTVKHLSLQIAATQPGD | |
| (substituted) | SAVYFCAERGRGGKLIFGQGTELSVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain with | MAGIRALFMYLWLQLDWVSRGESVGLHLPTLSVQEGDNSIINCAYSNSASDY | 1789 |
| alternative signal | FIWYKQESGKGPQFIIDIRSNMDKRQGQRVTVLLNKTVKHLSLQIAATQPGD | |
| peptide, Cα | SAVYFCAERGRGGKLIFGQGTELSVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain with | MHGIRALFMYLWLQLDWVSRGESVGLHLPTLSVQEGDNSIINCAYSNSASDY | 1790 |
| alternative signal | FIWYKQESGKGPQFIIDIRSNMDKRQGQRVTVLLNKTVKHLSLQIAATQPGD | |
| peptide, Cα | SAVYFCAERGRGGKLIFGQGTELSVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| CDR1β | DFQATT | 2781 |
| CDR2β | SNEGSKA | 2782 |
| CDR3β | SAGRASTDTQY | 2783 |
| Vβ without signal | GSGLGAVVSQHPSWVICKSGTSVKIECRSLDFQATTMFWYRQFPKQSLMLMA | 2784 |
| peptide (SignalP) | TSNEGSKATYEQGVEKDKFLINHASLTLSTLTVTSAHPEDSSFYICSAGRAS | |
| | TDTQYFGPGTRLTVL | |
| Vβ without signal | GAVVSQHPSWVICKSGTSVKIECRSLDFQATTMFWYRQFPKQSLMLMATSNE | 2785 |
| peptide (IMGT) | GSKATYEQGVEKDKFLINHASLTLSTLTVTSAHPEDSSFYICSAGRASTDTQ | |
| | YFGPGTRLTVL | |
| Vβ | MXLLLLLLGPGISLLLPGSLAGSGLGAVVSQHPSWVICKSGTSVKIECRSLD | 2786 |
| | FQATTMFWYRQFPKQSLMLMATSNEGSKATYEQGVEKDKFLINHASLTLSTL | 2787 |
| | TVTSAHPEDSSFYICSAGRASTDTQYFGPGTRLTVL | |
| | (X = any amino acid) | |
| β chain with WT signal | MLLLLLLLGPGISLLLPGSLAGSGLGAVVSQHPSWVICKSGTSVKIECRSLD | 2788 |
| peptide, Cβ | FQATTMFWYRQFPKQSLMLMATSNEGSKATYEQGVEKDKFLINHASLTLSTL | |
| (substituted) | TVTSAHPEDSSFYICSAGRASTDTQYFGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHERCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MALLLLLLGPGISLLLPGSLAGSGLGAVVSQHPSWVICKSGTSVKIECRSLD | 2789 |
| signal peptide, Cβ | FQATTMFWYRQFPKQSLMLMATSNEGSKATYEQGVEKDKFLINHASLTLSTL | |
| (substituted) | TVTSAHPEDSSFYICSAGRASTDTQYFGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MHLLLLLLGPGISLLLPGSLAGSGLGAVVSQHPSWVICKSGTSVKIECRSLD | 2790 |
| signal peptide, Cβ | FQATTMFWYRQFPKQSLMLMATSNEGSKATYEQGVEKDKFLINHASLTLSTL | |
| (substituted) | TVTSAHPEDSSFYICSAGRASTDTQYFGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |

In some embodiments, TCR079 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR079 interacts with the neoantigen in the context of an HLA-DPA1*01:03 chain and an HLA-DPB1*03:01 chain, as described in International Publication No. WO 2021/173902, incorporated herein by reference in its entirety.

TABLE 6CB
Amino acid sequences of TCR080

| | | SEQ |
|---|---|---|
| | | ID |
| Description | Sequence | NO: |
| CDR1α | NSASDY | 1791 |
| CDR2α | IRSNMDK | 1792 |
| CDR3α | AERGRGGKLI | 1793 |
| Vα without signal | ESVGLHLPTLSVQEGDNSIINCAYSNSASDYFIWYKQESGKGPQFIIDIRSN | 1794 |
| peptide (SignalP) | MDKRQGQRVTVLLNKTVKHLSLQIAATQPGDSAVYFCAERGRGGKLIFGQGT | |
| | ELSVKP | |
| Vα without signal | GESVGLHLPTLSVQEGDNSIINCAYSNSASDYFIWYKQESGKGPQFIIDIRS | 1795 |
| peptide (IMGT) | NMDKRQGQRVTVLLNKTVKHLSLQIAATQPGDSAVYFCAERGRGGKLIFGQG | |
| | TELSVKP | |
| Vα | MXGIRALFMYLWLQLDWVSRGESVGLHLPTLSVQEGDNSIINCAYSNSASDY | 1796 |
| | FIWYKQESGKGPQFIIDIRSNMDKRQGQRVTVLLNKTVKHLSLQIAATQPGD | 1797 |
| | SAVYFCAERGRGGKLIFGQGTELSVKP | |
| | (X = any amino acid) | |
| α chain with WT signal | MAGIRALFMYLWLQLDWVSRGESVGLHLPTLSVQEGDNSIINCAYSNSASDY | 1798 |
| peptide, Cα | FIWYKQESGKGPQFIIDIRSNMDKRQGQRVTVLLNKTVKHLSLQIAATQPGD | |
| (substituted) | SAVYFCAERGRGGKLIFGQGTELSVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α | MAGIRALFMYLWLQLDWVSRGESVGLHLPTLSVQEGDNSIINCAYSNSASDY | 1799 |
| chain with | FIWYKQESGKGPQFIIDIRSNMDKRQGQRVTVLLNKTVKHLSLQIAATQPGD | |
| alternative signal | SAVYFCAERGRGGKLIFGQGTELSVKPNIQNPEPAVYQLKDPRSQDSTLCLF | |
| peptide, Cα | TDFDSQINVPKTMESGTFITDKCVLDMKAMDSKSNGAIAWSNQTSFTCQDIF | |
| (substituted) | KETNATYPSSDVPCDATLTEKSFETDMNLNFQNLLVIVLRILLLKVAGENLL | |
| | MTLRLWSS | |
| α chain with | MHGIRALFMYLWLQLDWVSRGESVGLHLPTLSVQEGDNSIINCAYSNSASDY | 1800 |
| alternative signal | FIWYKQESGKGPQFIIDIRSNMDKRQGQRVTVLLNKTVKHLSLQIAATQPGD | |
| peptide, Cα | SAVYFCAERGRGGKLIFGQGTELSVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| CDR1β | DFQATT | 2791 |
| CDR2β | SNEGSKA | 2792 |
| CDR3β | SAGRASTDTQY | 2793 |
| Vβ without signal | AVVSQHPSWVICKSGTSVKIECRSLDFQATTMFWYRQFPKQSLMLMATSNEG | 2794 |
| peptide (SignalP) | SKATYEQGVEKDKFLINHASLTLSTLTVTSAHPEDSSFYICSAGRASTDTQY | |
| | FGPGTRLTVL | |
| Vβ without signal | GAVVSQHPSWVICKSGTSVKIECRSLDFQATTMFWYRQFPKQSLMLMATSNE | 2795 |
| peptide (IMGT) | GSKATYEQGVEKDKFLINHASLTLSTLTVTSAHPEDSSFYICSAGRASTDTQ | |
| | YFGPGTRLTVL | |
| Vβ | MXLLLLLLGPGSGLGAVVSQHPSWVICKSGTSVKIECRSLDFQATTMFWYRQ | 2796 |
| | FPKQSLMLMATSNEGSKATYEQGVEKDKFLINHASLTLSTLTVTSAHPEDSS | 2797 |
| | FYICSAGRASTDTQYFGPGTRLTVL | |
| | (X = any amino acid) | |
| β chain with WT signal | MLLLLLLLGPGSGLGAVVSQHPSWVICKSGTSVKIECRSLDFQATTMFWYRQ | 2798 |
| peptide, Cβ | FPKQSLMLMATSNEGSKATYEQGVEKDKFLINHASLTLSTLTVTSAHPEDSS | |
| (substituted) | FYICSAGRASTDTQYFGPGTRLTVLEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain with alternative | MALLLLLLGPGSGLGAVVSQHPSWVICKSGTSVKIECRSLDFQATTMFWYRQ | 2799 |
| signal peptide, Cβ | FPKQSLMLMATSNEGSKATYEQGVEKDKFLINHASLTLSTLTVTSAHPEDSS | |
| (substituted) | FYICSAGRASTDTQYFGPGTRLTVLEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain with alternative | MHLLLLLLGPGSGLGAVVSQHPSWVICKSGTSVKIECRSLDFQATTMFWYRQ | 2800 |
| signal peptide, Cβ | FPKQSLMLMATSNEGSKATYEQGVEKDKFLINHASLTLSTLTVTSAHPEDSS | |
| (substituted) | FYICSAGRASTDTQYFGPGTRLTVLEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR080 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR080 interacts with the neoantigen in the context of an HLA-DPA1*01:03 chain and an HLA-DPB1*03:01 chain, as described in International Publication No. WO 2021/173902, incorporated herein by reference in its entirety.

The disclosure also provides for the use of other TCR Vα and Vβ sequences, as well as any other alpha or beta chains, in the polycistronic vectors, engineered cells or pharmaceutical compositions described herein. These TCR Vα and Vβ sequences and alpha or beta chains include those described in International Publication Nos. WO 2016/085904, WO 2017/048593, WO 2018/026691, WO 2019/060349, WO 2019/067243, WO 2019/070435, WO 2019/112941, WO 2019/213195, WO 2020/086827, WO 2020/154275, WO 2020/264269, WO 2021/163434, WO 2021/163477, and WO 2021/173902 incorporated by reference herein in their entireties.

The CDRs of a TCR disclosed herein can be defined using any art recognized numbering convention. Additionally or alternatively, the CDRs can be defined empirically, e.g., based upon structural analysis of the interaction of the TCR with a cognate antigen (e.g., a peptide or a peptide-MHC complex). In some embodiments, CDR3 of the TCR can further comprise an N-terminal cysteine and/or a C-terminal phenylalanine or tryptophan.

The TCRs disclosed herein can be used in any TCR structural format. For example, in certain embodiments, the TCR is a full-length TCR comprising a full-length α chain and a full-length β chain. The transmembrane regions (and optionally also the cytoplasmic regions) can be removed from a full-length TCR to produce a soluble TCR. Accordingly, in certain embodiments, the TCR is a soluble TCR lacking transmembrane and/or cytoplasmic region(s). The methods of producing soluble TCRs are well-known in the art. In some embodiments, the soluble TCR comprises an engineered disulfide bond that facilitates dimerization, see, e.g., U.S. Pat. No. 7,329,731, which is incorporated by reference herein in its entirety. In some embodiments, the soluble TCR is generated by fusing the extracellular domain of a TCR described herein to other protein domains, e.g., maltose binding protein, thioredoxin, human constant kappa domain, or leucine zippers, see, e.g., Løset et al., Front Oncol. 2014; 4: 378, which is incorporated by reference herein in its entirety. A single-chain TCR (scTCR) comprising Vα and Vβ linked by a peptide linker can also be generated. Such scTCRs can comprise Vα and Vβ, each linked to a TCR constant region. Alternatively, the scTCRs can comprise Vα and VO, where either the Vα, the Vβ, or both the Vα and Vβ are not linked to a TCR constant region. Exemplary scTCRs are described in PCT Publication Nos. WO 2003/020763, WO 2004/033685, and WO 2011/044186, each of which is incorporated by reference herein in its entirety. Furthermore, the TCRs disclosed herein can comprise two polypeptide chains (e.g., an α chain and a β chain) in which the chains have been engineered to each have a cysteine residue that can form an interchain disulfide bond. Accordingly, in certain embodiments, the TCRs disclosed herein comprise two polypeptide chains linked by an engineered disulfide bond. Exemplary TCRs having an engineered disulfide bond are described in U.S. Pat. Nos. 8,361,794 and 8,906,383, each of which is incorporated by reference herein in its entirety.

In certain embodiments, the TCRs disclosed herein comprise one or more chains (e.g., an α chain and/or a β chain) having a transmembrane region. In certain embodiments, the TCRs disclosed herein comprise two chains (e.g., an α chain and a β chain) having a transmembrane region. The transmembrane region can be the endogenous transmembrane region of that TCR chain, a variant of the endogenous transmembrane region, or a heterologous transmembrane region. In certain embodiments, the TCRs disclosed herein comprise an α chain and a β chain having endogenous transmembrane regions.

In certain embodiments, the TCRs disclosed herein comprise one or more chains (e.g., an α chain and/or a β chain) having a cytoplasmic region. In certain embodiments, the TCRs disclosed herein comprise two chains (e.g., an α chain and a β chain) each having a cytoplasmic region. The cytoplasmic region can be the endogenous cytoplasmic region of that TCR chain, variant of the endogenous cytoplasmic region, or a heterologous cytoplasmic region. In certain embodiments, the TCRs disclosed herein comprise two chains (e.g., an α chain and a β chain) where both chains have transmembrane regions, but one chain is lacking a cytoplasmic region. In certain embodiments, the TCRs disclosed herein comprise two chains (e.g., an α chain and a β chain) where both chains have endogenous transmembrane regions but lack an endogenous cytoplasmic region. In certain embodiments, the TCRs disclosed herein comprise an α chain and a β chain where both chains have endogenous transmembrane regions but lack an endogenous cytoplasmic region. In certain embodiments, the TCRs disclosed herein comprise a co-stimulatory signaling region from a co-stimulatory molecule; see, e.g., PCT Publication Nos.: WO 1996/018105, WO 1999/057268, and WO 2000/031239, and U.S. Pat. No. 7,052,906, all of which are incorporated herein by reference in their entireties.

In certain embodiments, the instant disclosure provides a polypeptide comprising an a chain variable region (Vα) and a β chain variable region (Vβ) of a TCR fused together. For example, such polypeptide may comprise, in order, the Vα and Vβ, or the Vβ and the Vα, optionally with a linker (e.g., a peptide linker) between the two regions. For example, a Furin and/or a 2A cleavage site (e.g., one of the sequences in Tables 2 or 3), or combinations thereof, may be used in the linker for the Vα/Vβ fusion polypeptide. In certain embodiments, the instant disclosure provides a polypeptide comprising an α chain and a β chain of a TCR fused together. For example, such polypeptide may comprise, in order, an α chain and a β chain, or a β chain and an α chain, optionally with a linker (e.g., a peptide linker) between the two chains. For example, a Furin and/or a 2A cleavage site (e.g., one of the sequences in Tables 2 or 3), or combinations thereof, may be used in the linker for the a/s fusion polypeptide. For example, a fusion polypeptide may comprise, from the N-terminus to the C-terminus: the α chain of a TCR, a furin cleavage site, a 2A cleavage site, and the β chain of the TCR. In certain embodiments, the polypeptide comprises, from the N-terminus to the C-terminus: the β chain of a TCR, a furin cleavage site, a 2A element, and the α chain of the TCR.

### 5.3 Methods of Use

In another aspect, the instant disclosure provides a method of treating a subject using the polycistronic polynucleotides, recombinant vectors, engineered cells (e.g., a cell comprising a heterologous and/or recombinant nucleic acid), or pharmaceutical compositions disclosed herein. Any disease or disorder in a subject that would benefit from treatment with a recombinant cell of the present disclosure, or a polynucleotide or vector of the present disclosure can be treated using the methods disclosed herein.

In certain embodiments, the method comprises administering to the subject an effective amount of a recombinant cell or population thereof as disclosed herein.

As disclosed infra, cells administered to the subject can be autologous or allogeneic to the subject. In certain embodiments, autologous cells are obtained from a cancer patient directly following a cancer treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, in certain embodiments, cells are collected from blood, bone marrow, lymph node, thymus, or another tissue or bodily fluid, or an apheresis product, during this recovery phase. Further, in certain aspects, mobilization and conditioning regimens can be used to create a condition in a subject wherein repopulation, recirculation, regeneration, and/or expansion of particular cell types is favored, especially during a defined window of time following therapy.

The number of cells that are employed will depend upon a number of circumstances including, the lifetime of the cells, the protocol to be used (e.g., the number of administrations), the ability of the cells to multiply, the stability of the recombinant construct, and the like. In certain embodiments, the cells are applied as a dispersion, generally being injected at or near the site of interest. The cells may be administered in any physiologically acceptable medium.

In certain embodiments, the cancer is cancer of the lung, bile duct cancer (e.g., cholangiocarcinoma), pancreatic cancer, colorectal cancer, ovarian, or gynecologic cancer. In certain embodiments, the cancer is leukemia (e.g., mixed lineage leukemia, acute lymphocytic leukemia, acute myeloid leukemia, chronic lymphocytic leukemia, or chronic myeloid leukemia), alveolar rhabdomyosarcoma, bone cancer, brain cancer (e.g., glioma, e.g., glioblastoma), breast cancer, cancer of the anus, anal canal, or anorectum, cancer of the eye, cancer of the intrahepatic bile duct (e.g., intrahepatic cholangiocellular cancer), cancer of the joints, cancer of the neck, gallbladder, or pleura, cancer of the nose, nasal cavity, or middle ear, cancer of the oral cavity, cancer of the vulva, myeloma (e.g., chronic myeloid cancer), colon cancer, esophageal cancer, cervical cancer, gastrointestinal cancer, gastrointestinal carcinoid tumor, Hodgkin's lymphoma, hypopharynx cancer, kidney cancer, larynx cancer, liver cancer (e.g., hepatocellular carcinoma), lung cancer (e.g., non-small cell lung cancer), malignant mesothelioma, melanoma, multiple myeloma, nasopharynx cancer, non-Hodgkin's lymphoma, ovarian cancer, pancreatic cancer, peritoneum, omentum, and mesentery cancer, pharynx cancer, prostate cancer, rectal cancer, renal cancer (e.g., renal cell carcinoma (RCC)), gastric cancer, small intestine cancer, soft tissue cancer, stomach cancer, carcinoma, sarcoma (e.g., synovial sarcoma, rhabdomyosarcoma), skin cancer, testicular cancer, thyroid cancer, head and neck cancer, ureter cancer, and urinary bladder cancer. In certain embodiments, the cancer is melanoma, breast cancer, lung cancer, prostate cancer, thyroid cancer, ovarian cancer, or synovial sarcoma. In one embodiment, the cancer is synovial sarcoma or liposarcoma (e.g., myxoid/round cell liposarcoma). In certain embodiments, the cancer is lung, cholangiocarcinoma, pancreatic, colorectal, gynecological or ovarian cancer.

A polycistronic polynucleotide, recombinant vector, engineered cell, or pharmaceutical composition described herein may be delivered to a subject by a variety of routes. These include, but are not limited to, parenteral, intranasal, intratracheal, oral, intradermal, topical, intramuscular, intraperitoneal, transdermal, intravenous, intratumoral, conjunctival, intrathecal, and subcutaneous routes. Pulmonary administration can also be employed, e.g., by use of an inhaler or nebulizer, and formulation with an aerosolizing agent for use as a spray. In certain embodiments, the polycistronic polynucleotide, recombinant vector, engineered cell, or pharmaceutical composition described herein is delivered intravenously. In certain embodiments, the polycistronic polynucleotide, vector, engineered cell, or pharmaceutical composition described herein is delivered subcutaneously. In certain embodiments, the polycistronic polynucleotide, recombinant vector, engineered cell, or pharmaceutical composition described herein is delivered intratumorally. In certain embodiments, the polycistronic polynucleotide, recombinant vector, engineered cell, or pharmaceutical composition described herein is delivered into a tumor draining lymph node.

The amount of the polycistronic polynucleotide, recombinant vector, engineered cell, or pharmaceutical composition which will be effective in the treatment and/or prevention of a condition will depend on the nature of the disease, and can be determined by standard clinical techniques.

The precise dose to be employed in a composition will also depend on various factors, including but not limited to the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, and health), whether the patient is a human or an animal, other medications administered, or whether treatment is prophylactic or therapeutic. Usually, the patient is a human but non-human mammals including transgenic mammals can also be treated. Treatment dosages are optimally titrated to optimize safety and efficacy.

### 5.4 IL-15/IL-15Rα Fusion Proteins

The disclosure also provides recombinant vectors that include cytokines. In some embodiments, the cytokine is an interleukin. In some embodiments, the cytokine is membrane bound. In some embodiments, the cytokine is a fusion protein comprising a soluble cytokine, or a functional fragment or functional variant thereof, operably linked to a cognate receptor of the cytokine, or a functional fragment or functional variant thereof, optionally a membrane-bound form thereof. In some embodiments, the fusion protein comprises human IL-15 (hIL-15) operably linked to human IL-15Rα (hIL-15Rα). In membrane-bound form, this fusion protein is referred to herein as membrane bound IL-15 (mbIL15). In some embodiments, hIL-15 is directly operably linked to hIL-15Rα. In some embodiments, hIL-15 is indirectly operably linked to hIL-15Rα. In some embodiments, hIL-15 is indirectly operably linked to hIL-15Rα via a peptide linker.

In some embodiments, the peptide linker comprises the amino acid sequence of SEQ ID NO: 81, or an amino acid sequence comprising 1, 2, 3, 4 or 5 amino acid modifications to the amino acid sequence of SEQ ID NO: 81. In some embodiments, the linker comprises the amino acid sequence of SEQ ID NO: 81. In some embodiments, the amino acid of the linker consists of the amino acid sequence of SEQ ID NO: 81, or an amino acid sequence comprising 1, 2, 3, 4 or 5 amino acid modifications to the amino acid sequence of SEQ ID NO: 81. In some embodiments, the amino acid of the linker consists of the amino acid sequence of SEQ ID NO: 81.

In some embodiments, the linker is encoded by a polynucleotide sequence at least 95%, 96%, 97%, 98%, 99%, or 100% identical to the polynucleotide sequence of SEQ ID NO: 82. In some embodiments, the linker is encoded by the polynucleotide sequence of SEQ ID NO: 82. In some embodiments, hIL-15 comprises an amino acid sequence at least 95%, 96%, 97%, 98%, 99% or 100% identical to the amino acid sequence of SEQ ID NO: 76. In some embodiments, hIL-15 comprises the amino acid sequence of SEQ ID NO: 76. In some embodiments, the amino acid sequence of hIL-15 consists of a sequence at least 95%, 96%, 97%, 98%, 99% or 100% identical to the amino acid sequence of SEQ ID NO: 76. In some embodiments, the amino acid sequence of hIL-15 consists of the amino acid sequence of SEQ ID NO: 76.

In some embodiments, hIL-15 is encoded by a polynucleotide sequence at least 75%, 80%, 85%, 90%, 95%, 96%, 97%, 98%, 99%, or 100% identical to the polynucleotide sequence of SEQ ID NO: 77. In some embodiments, hIL-15 is encoded by the polynucleotide sequence of SEQ ID NO: 77.

In some embodiments, hIL-15Rα comprises an amino acid sequence at least 95%, 96%, 97%, 98%, 99% or 100% identical to the amino acid sequence of SEQ ID NO: 78. In some embodiments, hIL-15Rα comprises the amino acid sequence of SEQ ID NO: 78. In some embodiments, the amino acid sequence of hIL-15Rα consists of a sequence at least 95%, 96%, 97%, 98%, 99% or 100% identical to the amino acid sequence of SEQ ID NO: 78. In some embodiments, the amino acid sequence of hIL-15Rα consists of the amino acid sequence of SEQ ID NO: 78.

In some embodiments, hIL-15Rα is encoded by a polynucleotide sequence at least 75%, 80%, 85%, 90%, 95%, 96%, 97%, 98%, 99%, or 100% identical to the polynucleotide sequence of SEQ ID NO: 79. In some embodiments, hIL-15Rα is encoded by the polynucleotide sequence of SEQ ID NO: 79

In some embodiments, the fusion protein comprises an amino acid sequence at least 95%, 96%, 97%, 98%, 99% or 100% identical to SEQ ID NO: 70 or 73. In some embodiments, the fusion protein comprises an amino acid sequence at least 95%, 96%, 97%, 98%, 99% or 100% identical to SEQ ID NO: 70. In some embodiments, the fusion protein comprises an amino acid sequence at least 95%, 96%, 97%, 98%, 99% or 100% identical to SEQ ID NO: 73. In some embodiments, the fusion protein comprises the amino acid sequence of SEQ ID NO: 70 or 73. In some embodiments, the fusion protein comprises the amino acid sequence of SEQ ID NO: 70. In some embodiments, the fusion protein comprises the amino acid sequence of SEQ ID NO: 73.

In some embodiments, the amino acid sequence of the fusion protein consists of a sequence at least 95%, 96%, 97%, 98%, 99% or 100% identical to the amino acid sequence of SEQ ID NO: 70 or 73. In some embodiments, the amino acid sequence of the fusion protein consists of a sequence at least 95%, 96%, 97%, 98%, 99% or 100% identical to the amino acid sequence of SEQ ID NO: 70. In some embodiments, the amino acid sequence of the fusion protein consists of a sequence at least 95%, 96%, 97%, 98%, 99% or 100% identical to the amino acid sequence of SEQ ID NO: 73. In some embodiments, the amino acid sequence of the fusion protein consists of the amino acid sequence of SEQ ID NO: 70 or 73. In some embodiments, the amino acid sequence of the fusion protein consists of the amino acid sequence of SEQ ID NO: 70. In some embodiments, the amino acid sequence of the fusion protein consists of the amino acid sequence of SEQ ID NO: 73.

In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 71 or 74. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 71. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 74.

In some embodiments, the fusion protein is encoded by the polynucleotide sequence of SEQ ID NO: 71 or 74. In some embodiments, the fusion protein is encoded by the polynucleotide sequence of SEQ ID NO: 71. In some embodiments, the fusion protein is encoded by the polynucleotide sequence of SEQ ID NO: 74.

Exemplary cytokine fusion proteins and components thereof are disclosed in Table 7. Additional exemplary mbIL15 fusions are disclosed in Hurton et al., “Tethered IL-15 augments antitumor activity and promotes a stem-cell memory subset in tumor-specific T cells,” PNAS, 113(48) E7788-E7797 (2016), the entire contents of which are incorporated by reference herein.

The amino acid sequence and polynucleotide sequence of exemplary cytokine fusion proteins and component polypeptides are provided in Table 7, herein.

TABLE 7
Amino acid and polynucleotide sequences of exemplary IL-15/IL-15Rα fusion
proteins and thereof.

| | | SEQ ID |
|---|---|---|
| Description | Sequence | NO |
| mbIL15 (with N- | MDWTWILFLVAAATRVHSNWVNVISDLKKIEDLIQSMHIDATLYTESDVHPSCK | 70 |
| terminal signal | VTAMKCFLLELQVISLESGDASIHDTVENLIILANNSLSSNGNVTESGCKECEE | |
| sequence) | LEEKNIKEFLQSFVHIVQMFINTSSGGGSGGGGSGGGGSGGGGSGGGSLQITCP | |
| (exemplary amino | PPMSVEHADIWVKSYSLYSRERYICNSGFKRKAGTSSLTECVLNKATNVAHWTT | |
| acid sequence) | PSLKCIRDPALVHQRPAPPSTVTTAGVTPQPESLSPSGKEPAASSPSSNNTAAT | |
| | TAAIVPGSQLMPSKSPSTGTTEISSHESSHGTPSQTTAKNWELTASASHQPPGV | |
| | YPQGHSDTTVAISTSTVLLCGLSAVSLLACYLKSRQTPPLASVEMEAMEALPVT | |
| | WGTSSRDEDLENCSHHL | |
| mbIL15 (with N- | ATGGATTGGACCTGGATTCTGTTTCTGGTGGCCGCTGCCACAAGAGTGCACAGC | 71 |
| terminal signal | AACTGGGTGAATGTGATCAGCGACCTGAAGAAGATCGAGGATCTGATCCAGAGC | |
| sequence) | ATGCACATTGATGCCACCCTGTACACAGAATCTGATGTGCACCCTAGCTGTAAA | |
| (exemplary | GTGACCGCCATGAAGTGTTTTCTGCTGGAGCTGCAGGTGATTTCTCTGGAAAGC | |
| nucleotide | GGAGATGCCTCTATCCACGACACAGTGGAGAATCTGATCATCCTGGCCAACAAT | |
| sequence) | AGCCTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGAGGA | |
| | GGAGGCAGTGGAGGCGGAGGATCTGGCGGAGGATCTCTGCAGATTACATGCCCT | |
| | CCTCCAATGTCTGTGGAGCACGCCGATATTTGGGTGAAGTCCTACAGCCTGTAC | |
| | AGCAGAGAGAGATACATCTGCAACAGCGGCTTTAAGAGAAAGGCCGGCACCTCT | |
| | TCTCTGACAGAGTGCGTGCTGAATAAGGCCACAAATGTGGCCCACTGGACAACA | |
| | CCTAGCCTGAAGTGCATTAGAGATCCTGCCCTGGTCCACCAGAGGCCTGCCCCT | |
| | CCATCTACAGTGACAACAGCCGGAGTGACACCTCAGCCTGAATCTCTGAGCCCT | |
| | TCTGGAAAAGAACCTGCCGCCAGCTCTCCTAGCTCTAATAATACCGCCGCCACA | |
| | ACAGCCGCCATTGTGCCTGGATCTCAGCTGATGCCTAGCAAGTCTCCTAGCACA | |
| | GGCACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGAGTG | |
| | TATCCTCAGGGCCACTCTGATACAACAGTGGCCATCAGCACATCTACAGTGCTG | |
| | CTGTGTGGACTGTCTGCCGTGTCTCTGCTGGCCTGTTACCTGAAGTCTAGACAG | |
| | ACACCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| mbIL15 (without | NWVNVISDLKKIEDLIQSMHIDATLYTESDVHPSCKVTAMKCFLLELQVISLES | 73 |
| N-terminal signal | GDASIHDTVENLIILANNSLSSNGNVTESGCKECEELEEKNIKEFLQSFVHIVQ | |
| sequence) | MFINTSSGGGSGGGGSGGGGSGGGGSGGGSLQITCPPPMSVEHADIWVKSYSLY | |
| (exemplary amino | SRERYICNSGFKRKAGTSSLTECVLNKATNVAHWTTPSLKCIRDPALVHQRPAP | |
| acid sequence) | PSTVTTAGVTPQPESLSPSGKEPAASSPSSNNTAATTAAIVPGSQLMPSKSPST | |
| | GTTEISSHESSHGTPSQTTAKNWELTASASHQPPGVYPQGHSDTTVAISTSTVL | |
| | LCGLSAVSLLACYLKSRQTPPLASVEMEAMEALPVTWGTSSRDEDLENCSHHL | |
| mbIL15 (without | AACTGGGTGAATGTGATCAGCGACCTGAAGAAGATCGAGGATCTGATCCAGAGC | 74 |
| N-terminal signal | ATGCACATTGATGCCACCCTGTACACAGAATCTGATGTGCACCCTAGCTGTAAA | |
| sequence) | GTGACCGCCATGAAGTGTTTTCTGCTGGAGCTGCAGGTGATTTCTCTGGAAAGC | |
| (exemplary | GGAGATGCCTCTATCCACGACACAGTGGAGAATCTGATCATCCTGGCCAACAAT | |
| nucleotide | AGCCTGAGCAGCAATGGCAATGTGACAGAGTCTGGCTGTAAGGAGTGTGAGGAG | |
| sequence) | CTGGAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGAGGA | |
| | GGAGGCAGTGGAGGCGGAGGATCTGGCGGAGGATCTCTGCAGATTACATGCCCT | |
| | CCTCCAATGTCTGTGGAGCACGCCGATATTTGGGTGAAGTCCTACAGCCTGTAC | |
| | AGCAGAGAGAGATACATCTGCAACAGCGGCTTTAAGAGAAAGGCCGGCACCTCT | |
| | TCTCTGACAGAGTGCGTGCTGAATAAGGCCACAAATGTGGCCCACTGGACAACA | |
| | CCTAGCCTGAAGTGCATTAGAGATCCTGCCCTGGTCCACCAGAGGCCTGCCCCT | |
| | CCATCTACAGTGACAACAGCCGGAGTGACACCTCAGCCTGAATCTCTGAGCCCT | |
| | TCTGGAAAAGAACCTGCCGCCAGCTCTCCTAGCTCTAATAATACCGCCGCCACA | |
| | ACAGCCGCCATTGTGCCTGGATCTCAGCTGATGCCTAGCAAGTCTCCTAGCACA | |
| | GGCACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGAGTG | |
| | TATCCTCAGGGCCACTCTGATACAACAGTGGCCATCAGCACATCTACAGTGCTG | |
| | CTGTGTGGACTGTCTGCCGTGTCTCTGCTGGCCTGTTACCTGAAGTCTAGACAG | |
| | ACACCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| Soluble hIL-15 | NWVNVISDLKKIEDLIQSMHIDATLYTESDVHPSCKVTAMKCFLLELQVISLES | 76 |
| (exemplary amino | GDASIHDTVENLIILANNSLSSNGNVTESGCKECEELEEKNIKEFLQSFVHIVQ | |
| acid sequence) | MFINTS | |
| Soluble hIL-15 | AACTGGGTGAATGTGATCAGCGACCTGAAGAAGATCGAGGATCTGATCCAGAGC | 77 |
| (exemplary | ATGCACATTGATGCCACCCTGTACACAGAATCTGATGTGCACCCTAGCTGTAAA | |
| nucleotide | GTGACCGCCATGAAGTGTTTTCTGCTGGAGCTGCAGGTGATTTCTCTGGAAAGC | |
| sequence) | GGAGATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTGCAG | |
| | ATGTTCATCAATACAAGC | |
| hIL-15Rα | ITCPPPMSVEHADIWVKSYSLYSRERYICNSGFKRKAGTSSLTECVLNKATNVA | 78 |
| (exemplary amino | HWTTPSLKCIRDPALVHQRPAPPSTVTTAGVTPQPESLSPSGKEPAASSPSSNN | |
| acid sequence) | TAATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAMEA | |
| | LPVTWGTSSRDEDLENCSHHL | |
| hIL-15Rα | ATTACATGCCCTCCTCCAATGTCTGTGGAGCACGCCGATATTTGGGTGAAGTCC | 79 |
| (exemplary | TACAGCCTGTACAGCAGAGAGAGATACATCTGCAACAGCGGCTTTAAGAGAAAG | |
| nucleotide | GCCGGCACCTCTTCTCTGACAGAGTGCGTGCTGAATAAGGCCACAAATGTGGCC | |
| sequence) | CACTGGACAACACCTAGCCTGAAGTGCATTAGAGATCCTGCCCTGGTCCACCAG | |
| | AGGCCTGCCCCTCCATCTACAGTGACAACAGCCGGAGTGACACCTCAGCCTGAA | |
| | TCTCTGAGCCCTTCTGGAAAAGAACCTGCCGCCAGCTCTCCTAGCTCTAATAAT | |
| | ACCGCCGCCACAACAGCCGCCATTGTGCCTGGATCTCAGCTGATGCCTAGCAAG | |
| | TCTCCTAGCACAGGCACAACAGAGATCAGCAGCCACGAATCTTCTCACGGAACA | |
| | CCTTCTCAGACCACCGCCAAGAATTGGGAGCTGACAGCCTCTGCCTCTCACCAG | |
| | CCTCCAGGAGTGTATCCTCAGGGCCACTCTGATACAACAGTGGCCATCAGCACA | |
| | TCTACAGTGCTGCTGTGTGGACTGTCTGCCGTGTCTCTGCTGGCCTGTTACCTG | |
| | AAGTCTAGACAGACACCTCCTCTGGCCTCTGTGGAGATGGAGGCCATGGAAGCC | |
| | CTGCCTGTGACATGGGGAACAAGCAGCAGAGATGAAGACCTGGAGAATTGTTCT | |
| | CACCACCTG | |
| Linker | SGGGSGGGGSGGGGSGGGGSGGGSLQ | 81 |
| (exemplary amino | | |
| acid sequence) | | |
| Linker | TCTGGCGGAGGATCTGGAGGAGGCGGATCTGGAGGAGGAGGCAGTGGAGGCGGA | 82 |
| (exemplary | GGATCTGGCGGAGGATCTCTGCAG | |
| nucleotide | | |
| sequence) | | |
| IgE N-terminal | MDWTWILFLVAAATRVHS | 83 |
| signal sequence | | |
| (exemplary amino | | |
| acid sequence) | | |
| IgE N-terminal | ATGGATTGGACCTGGATTCTGTTTCTGGTGGCCGCTGCCACAAGAGTGCACAGC | 84 |
| signal sequence | | |
| (exemplary | | |
| nucleotide | | |
| sequence) | | |

### 5.5 Marker Proteins

The marker proteins described herein function to allow for the selective depletion of cells contacted with the recombinant vector disclosed herein (e.g., “recombinant cells”) in vivo, through the administration of an agent, e.g., an antibody, that specifically binds to the marker protein and may mediate or catalyze killing of a recombinant cell. In some embodiments, marker proteins are expressed on the surface of the recombinant cell.

In some embodiments, the marker protein comprises the extracellular domain of a cell surface protein, or a functional fragment or functional variant thereof. In some embodiments, the cell surface protein is human epidermal growth factor receptor 1 (hHER1). In some embodiments, the marker protein comprises a truncated HER1 protein that is able to be bound by an anti-hHER1 antibody. In some embodiments, the marker protein comprises a variant of a truncated hHER1 protein that is able to be bound by an anti-hHER1 antibody. In some embodiments, the hHER1 marker protein provides a safety mechanism by allowing for depletion of infused recombinant cells through administering an antibody that recognizes the hHER1 marker protein expressed on the surface of recombinant cells. An exemplary antibody that binds the hHER1 marker protein is cetuximab.

In some embodiments, the hHER1 marker protein comprises from N terminus to C terminus: domain III of hHER1, or a functional fragment or functional variant thereof, an N-terminal portion of domain IV of hHER1; and the transmembrane region of human CD28.

In some embodiments, domain III of hHER1 comprises the amino acid sequence of SEQ ID NO: 104; or the amino acid sequence of SEQ ID NO: 104, comprising 1, 2, or 3 amino acid modifications. In some embodiments, the amino acid sequence of domain III of hHER1 consists of the amino acid sequence of SEQ ID NO: 104; or the amino acid sequence of SEQ ID NO: 10, comprising 1, 2, or 3 amino acid modifications.

In some embodiments, the N-terminal portion of domain IV of hHER1 comprises amino acids 1-40, 1-39, 1-38, 1-37, 1-36, 1-35, 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, or 1-10 of SEQ ID NO: 105. In some embodiments, the C terminus of domain III of hHER1 is directly fused to the N terminus of the N-terminal portion of domain IV of hHER1.

In some embodiments, the C terminus of the N-terminal portion of domain IV of hHER1 is indirectly fused to the N terminus of the CD28 transmembrane domain via a peptide linker. In some embodiments, the peptide linker comprises glycine and serine amino acid residues. In some embodiments, the peptide linker is from about 5-25, 5-20, 5-15, 5-10, 10-20, or 10-15 amino acids in length.

In some embodiments, the peptide linker comprises the amino acid sequence of SEQ ID NO: 108, or an amino acid sequence comprising 1, 2, 3, 4 or 5 amino acid modifications to the amino acid sequence of SEQ ID NO: 108. In some embodiments, the peptide linker comprises the amino acid sequence of SEQ ID NO: 108. In some embodiments, the amino acid sequence of the peptide linker consists of the amino acid sequence of SEQ ID NO: 108, or an amino acid sequence comprising 1, 2, 3, 4 or 5 amino acid modifications to the amino acid sequence of SEQ ID NO: 108. In some embodiments, the amino acid sequence of the peptide linker consists of the amino acid sequence of SEQ ID NO: 108.

In some embodiments, the marker protein comprises an amino acid sequence at least 95%, 96%, 97%, 98%, 99% or 100% identical to the amino acid sequence of SEQ ID NO: 100, 103, 112, or 113. In some embodiments, the marker protein comprises an amino acid sequence at least 95%, 96%, 97%, 98%, 99% or 100% identical to the amino acid sequence of SEQ ID NO: 100. In some embodiments, the marker protein comprises an amino acid sequence at least 95%, 96%, 97%, 98%, 99% or 100% identical to the amino acid sequence of SEQ ID NO: 103. In some embodiments, the marker protein comprises an amino acid sequence at least 95%, 96%, 97%, 98%, 99% or 100% identical to the amino acid sequence of SEQ ID NO: 112. In some embodiments, the marker protein comprises an amino acid sequence at least 95%, 96%, 97%, 98%, 99% or 100% identical to the amino acid sequence of SEQ ID NO: 113.

In some embodiments, the marker protein comprises the amino acid sequence of SEQ ID NO: 100 or 103. In some embodiments, the marker protein comprises the amino acid sequence of SEQ ID NO: 100. In some embodiments, the marker protein comprises the amino acid sequence of SEQ ID NO: 103.

In some embodiments, the marker protein consists of an amino acid sequence at least 95%, 96%, 97%, 98%, 99% or 100% identical to the amino acid sequence of SEQ ID NO: 100, 103, 112, or 113. In some embodiments, the marker protein consists of an amino acid sequence at least 95%, 96%, 97%, 98%, 99% or 100% identical to the amino acid sequence of SEQ ID NO: 100. In some embodiments, the marker protein consists of an amino acid sequence at least 95%, 96%, 97%, 98%, 99% or 100% identical to the amino acid sequence of SEQ ID NO: 103. In some embodiments, the marker protein consists of an amino acid sequence at least 95%, 96%, 97%, 98%, 99% or 100% identical to the amino acid sequence of SEQ ID NO: 112. In some embodiments, the marker protein consists of an amino acid sequence at least 95%, 96%, 97%, 98%, 99% or 100% identical to the amino acid sequence of SEQ ID NO: 113.

In some embodiments, the marker protein consists of the amino acid sequence of SEQ ID NO: 100, 103, 112, or 113. In some embodiments, the marker protein consists of the amino acid sequence of SEQ ID NO: 100. In some embodiments, the marker protein consists of the amino acid sequence of SEQ ID NO: 103. In some embodiments, the marker protein consists of the amino acid sequence of SEQ ID NO: 112. In some embodiments, the marker protein consists of the amino acid sequence of SEQ ID NO: 113.

In some embodiments, the marker protein is derived from human CD20 (hCD20). In some embodiments, the marker protein comprises a truncated hCD20 protein that comprises the extracellular region (hCD20t), or a functional fragment or functional variant thereof. In some embodiments, the hCD20 marker protein provides a safety mechanism by allowing for depletion of infused recombinant cells through administering an antibody that recognizes the hCD20 marker protein expressed on the surface of recombinant cells. An exemplary antibody that binds the hCD20 marker protein is rituximab.

The amino acid sequences of exemplary marker proteins are provided in Table 8, herein.

TABLE 8
Amino acid sequences of exemplary marker proteins.

| | | SEQ ID |
|---|---|---|
| Description | Amino Acid Sequence | NO |
| HER1t (with N-terminal | MRLPAQLLGLLMLWVPGSSGRKVCNGIGIGEFKDSLSINATNI | 100 |
| signal sequence) (exemplary | KHFKNCTSISGDLHILPVAFRGDSFTHTPPLDPQELDILKTVK | |
| amino acid sequence) | EITGELLIQAWPENRTDLHAFENLEIIRGRTKQHGQFSLAVVS | |
| | LNITSLGLRSLKEISDGDVIISGNKNLCYANTINWKKLFGTSG | |
| | QKTKIISNRGENSCKATGQVCHALCSPEGCWGPEPRDCVSGGG | |
| | GSGGGGSGGGGSGGGGSFWVLVVVGGVLACYSLLVTVAFIIFW | |
| | VRSKRS | |
| HERIt (without N-terminal | RKVCNGIGIGEFKDSLSINATNIKHFKNCTSISGDLHILPVAF | 103 |
| signal sequence) (exemplary | RGDSFTHTPPLDPQELDILKTVKEITGELLIQAWPENRTDLHA | |
| amino acid sequence) | FENLEIIRGRTKQHGQFSLAVVSLNITSLGLRSLKEISDGDVI | |
| | ISGNKNLCYANTINWKKLFGTSGQKTKIISNRGENSCKATGQV | |
| | CHALCSPEGCWGPEPRDCVSGGGGSGGGGSGGGGSGGGGSFWV | |
| | LVVVGGVLACYSLLVTVAFIIFWVRSKRS | |
| Domain III of hHER1 | RKVCNGIGIGEFKDSLSINATNIKHFKNCTSISGDLHILPVAF | 104 |
| (exemplary amino acid | RGDSFTHTPPLDPQELDILKTVKEITGFLLIQAWPENRTDLHA | |
| sequence) | FENLEIIRGRTKQHGQFSLAVVSLNITSLGLRSLKEISDGDVI | |
| | ISGNKNLCYANTINWKKLFGTSGQKTKIISNRGENSCKATGQ | |
| Domain IV of hHER1 | VCHALCSPEGCWGPEPRDCVSCRNVSRGRECVDKCNLLEGEPR | 105 |
| (exemplary amino acid | EFVENSECIQCHPECLPQAMNITCTGRGPDNCIQCAHYIDGPH | |
| sequence) | CVKTCPAGVMGENNTLVWKYADAGHVCHLCHPNCTYGCTGPGL | |
| | EGCPTNGPKIPS | |
| Truncated domain IV of | VCHALCSPEGCWGPEPRDCVS | 106 |
| hHER1 (exemplary amino | | |
| sequence) | | |
| CD28 transmembrane domain | FWVLVVVGGVLACYSLLVTVAFIIFWVRSKRS | 107 |
| (exemplary amino acid | | |
| sequence) | | |
| Linker (exemplary amino acid | GGGGSGGGGSGGGGSGGGGS | 108 |
| sequence) | | |
| Igκ N-terminal signal | MRLPAQLLGLLMLWVPGSSG | 109 |
| sequence (exemplary amino | | |
| acid sequence) | | |
| Igκ Variant 1 N-terminal | MRMRLPAQLLGLLMLWVPGSSG | 110 |
| signal sequence (exemplary | | |
| amino acid sequence) | | |
| Igκ Variant 2 N-terminal | PRMRLPAQLLGLLMLWVPGSSG | 111 |
| signal sequence (exemplary | | |
| amino acid sequence) | | |
| HER1t-2 (with N-terminal | MRLPAQLLGLLMLWVPGSSGRKVCNGIGIGEFKDSLSINATNI | 112 |
| signal sequence) (exemplary | KHFKNCTSISGDLHILPVAFRGDSFTHTPPLDPQELDILKTVK | |
| amino acid sequence) | EITGFLLIQAWPENRTDLHAFENLEIIRGRTKQHGQFSLAVVS | |
| | LNITSLGLRSLKEISDGDVIISGNKNLCYANTINWKKLFGTSG | |
| | QKTKIISNRGENSCKATGQVCHALCSPEGCWGPEPRDCVSCRN | |
| | VSRGRECVDKCNLLEGEPREFVENSECIQCHPECLPQAMNITC | |
| | TGRGPDNCIQCAHYIDGPHCVKTCPAGVMGENNTLVWKYADAG | |
| | HVCHLCHPNCTYGCTGPGLEGCPTNGPKIPSIATGMVGALLLL | |
| | LVVALGIGLFM | |
| HER1t-2 (without N-terminal | RKVCNGIGIGEFKDSLSINATNIKHFKNCTSISGDLHILPVAF | 113 |
| signal sequence) (exemplary | RGDSFTHTPPLDPQELDILKTVKEITGELLIQAWPENRTDLHA | |
| amino acid sequence) | FENLEIIRGRTKQHGQFSLAVVSLNITSLGLRSLKEISDGDVI | |
| | ISGNKNLCYANTINWKKLFGTSGQKTKIISNRGENSCKATGQV | |
| | CHALCSPEGCWGPEPRDCVSCRNVSRGRECVDKCNLLEGEPRE | |
| | FVENSECIQCHPECLPQAMNITCTGRGPDNCIQCAHYIDGPHC | |
| | VKTCPAGVMGENNTLVWKYADAGHVCHLCHPNCTYGCTGPGLE | |
| | GCPTNGPKIPSIATGMVGALLLLLVVALGIGLFM | |
| hCD20 (full length) | MTTPRNSVNGTFPAEPMKGPIAMQSGPKPLFRRMSSLVGPTQS | 114 |
| (exemplary amino acid | FFMRESKTLGAVQIMNGLFHIALGGLLMIPAGIYAPICVTVWY | |
| sequence) | PLWGGIMYIISGSLLAATEKNSRKCLVKGKMIMNSLSLFAAIS | |
| | GMILSIMDILNIKISHFLKMESLNFIRAHTPYINIYNCEPANP | |
| | SEKNSPSTQYCYSIQSLFLGILSVMLIFAFFQELVIAGIVENE | |
| | WKRTCSRPKSNIVLLSAEEKKEQTIEIKEEVVGLTETSSQPKN | |
| | EEDIEIIPIQEEEEEETETNFPEPPQDQESSPIENDSSP | |
| hCD20t-1 (exemplary amino | MTTPRNSVNGTFPAEPMKGPIAMQSGPKPLFRRMSSLVGPTQS | 115 |
| acid sequence) | FFMRESKTLGAVQIMNGLFHIALGGLLMIPAGIYAPICVTVWY | |
| | PLWGGIMYIISGSLLAATEKNSRKCLVKGKMIMNSLSLFAAIS | |
| | GMILSIMDILNIKISHFLKMESLNFIRAHTPYINIYNCEPANP | |
| | SEKNSPSTQYCYSIQSLFLGILSVMLIFAFFQELVIAGIVENE | |
| | WKRTCSRPKSNIVLLSAEEKKEQTIEIKEEVVGLTETSSQPKN | |
| | EEDIE | |

### 5.6 Vectors

In one aspect, provided herein are recombinant vectors comprising a polycistronic expression cassette that comprises at least three cistrons. In some embodiments, the polycistronic expression cassette comprises at least 4, 5, or 6 cistrons. In some embodiments, the polycistronic expression cassette comprises 3 cistrons. In some embodiments, the polycistronic expression cassette comprises 4 cistrons. In some embodiments, the polycistronic expression cassette comprises 5 cistrons. In some embodiments, the polycistronic expression cassette comprises 6 cistrons.

In some embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In some embodiments, the polycistronic vector is a DNA plasmid vector.

In some embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV2, 3, 5, 6, 8, 9), retrovirus vectors (MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector (e.g., MV-Edm), Newcastle disease virus vectors, poxvirus vectors (e.g., VV), measles virus, and picornavirus vectors (e.g., Coxsackievirus).

In some embodiments, the vector or polycistronic expression cassette comprises one or more additional elements. Additional elements include, but are not limited to, promoters, enhancers, polyadenylation (polyA) sequences, and selection genes.

In some embodiments, the vector comprises a polynucleotide sequence that encodes for a selectable marker that confers a specific trait on cells in which the selectable marker is expressed enabling artificial selection of those cells. Exemplary selectable markers include, but are not limited to, antibiotic resistance genes, e.g., resistance to kanamycin, ampicillin, or triclosan.

In some embodiments, the polycistronic expression cassette comprises a transcriptional regulatory element. Exemplary transcriptional regulatory elements include, but are not limited to promoters and enhancers. In some embodiments, the polycistronic expression cassette comprises a promoter sequence 5′ of the first 5′ cistron. In some embodiments, the promoter comprises a polynucleotide sequence at least 75%, 80%, 85%, 90%, 95%, 96%, 97%, 98%, 99%, or 100% identical to the polynucleotide sequence of SEQ ID NO: 150. In some embodiments, the promoter comprises the polynucleotide sequence of SEQ ID NO: 150. In some embodiments, the polynucleotide sequence of the promoter consists of a polynucleotide sequence at least 75%, 80%, 85%, 90%, 95%, 96%, 97%, 98%, 99%, or 100% identical to the polynucleotide sequence of SEQ ID NO: 150. In some embodiments, the polynucleotide sequence of the promoter consists of the polynucleotide sequence of SEQ ID NO: 150.

In some embodiments, the polycistronic expression cassette comprises a polyA sequence 3′ of the 3′ terminal cistron. In some embodiments, the polyA sequence comprises a polynucleotide sequence at least 75%, 80%, 85%, 90%, 95%, 96%, 97%, 98%, 99%, or 100% identical to the polynucleotide sequence of SEQ ID NO: 151. In some embodiments, the polyA sequence comprises the nucleic acid sequence of SEQ ID NO: 151. In some embodiments, the polyA sequence consists of a sequence at least 75%, 80%, 85%, 90%, 95%, 96%, 97%, 98%, 99%, or 100% identical to the polynucleotide sequence of SEQ ID NO: 151. In some embodiments, the polyA sequence consists of the nucleic acid sequence of SEQ ID NO: 151.

The polynucleotide sequence of exemplary promoters and polyA sequences are provided in Table 9, herein.

TABLE 9
sequences of exemplary promoters and polyA sequences.

| Description | Nucleic Acid Sequence | SEQ ID NO |
|---|---|---|
| hEF-1α | GGATCTGCGATCGCTCCGGTGCCCGTCAGTGGGCAGAGCGCACATC | 150 |
| Hybrid | GCCCACAGTCCCCGAGAAGTTGGGGGGAGGGGTCGGCAATTGAACC | |
| Promoter | GGTGCCTAGAGAAGGTGGCGCGGGGTAAACTGGGAAAGTGATGTCG | |
| | TGTACTGGCTCCGCCTTTTTCCCGAGGGTGGGGGAGAACCGTATAT | |
| | AAGTGCAGTAGTCGCCGTGAACGTTCTTTTTCGCAACGGGTTTGCC | |
| | GCCAGAACACAGCTGAAGCTTCGAGGGGCTCGCATCTCTCCTTCAC | |
| | GCGCCCGCCGCCCTACCTGAGGCCGCCATCCACGCCGGTTGAGTCG | |
| | CGTTCTGCCGCCTCCCGCCTGTGGTGCCTCCTGAACTGCGTCCGCC | |
| | GTCTAGGTAAGTTTAAAGCTCAGGTCGAGACCGGGCCTTTGTCCGG | |
| | CGCTCCCTTGGAGCCTACCTAGACTCAGCCGGCTCTCCACGCTTTG | |
| | CCTGACCCTGCTTGCTCAACTCTACGTCTTTGTTTCGTTTTCTGTT | |
| | CTGCGCCGTTACAGATCCAAGCTGTGACCGGCGCCTACCTGAGAT | |
| BGH | GATCTGCTGTGCCTTCTAGTTGCCAGCCATCTGTTGTTTGCCCCTC | 151 |
| poly A | CCCCGTGCCTTCCTTGACCCTGGAAGGTGCCACTCCCACTGTCCTT | |
| sequence | TCCTAATAAAATGAGGAAATTGCATCGCATTGTCTGAGTAGGTGTC | |
| | ATTCTATTCTGGGGGGTGGGGTGGGGCAGGACAGCAAGGGGGAGGA | |
| | TTGGGAAGACAATAGCAGGCATGCTGGGGATGCGGTGGGCTCTATG | |
| | G | |

In some embodiments, the polycistronic expression cassette comprises a polynucleotide sequence that encodes an amino acid sequence at least 90%, 95%, 96%, 97%, 98%, 99% or 100% identical to an amino acid sequence recited in Tables 10A-10C.

TABLE 10A
Exemplary amino acid sequences encoded by polycistronic expression cassettes.

| | | SEQ ID |
|---|---|---|
| Description | Sequence | NO: |
| Cα (murine, | XIQNPEPAVYQLKDPRSQDSTLCLFTDFDSQINVPKTMESGTFITDKXVLDM | 160 |
| degenerate)-fP2A | KAMDSKSNGAIAWSNQTSFTCQDIFKETNATYPSSDVPCDATLTEKSFETDM | |
| | NLNFQNLXVXXLRILLLKVAGFNLLMTLRLWSSRAKRSGSGATNFSLLKQAG | |
| | DVEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 161 |
| degenerate)-fT2A- | HSGVXTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHL | |
| | X at position 57 is Ser or Cys | |
| Cα (murine, | XIQNPEPAVYQLKDPRSQDSTLCLFTDFDSQINVPKTMESGTFITDKXVLDM | 162 |
| degenerate)-fT2A | KAMDSKSNGAIAWSNQTSFTCQDIFKETNATYPSSDVPCDATLTEKSFETDM | |
| | NLNFQNLXVXXLRILLLKVAGENLLMTLRLWSSRAKRSGSGEGRGSLLTCGD | |
| | VEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLEEPSKAEIANKQKATLVCLARGFFPDHVELSWWVNGKEV | 163 |
| degenerate)-fP2A- | HSGVXTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHL | |
| | X at position 57 is Ser or Cys | |
| Cα (murine, | XIQNPEPAVYQLKDPRSQDSTLCLFTDFDSQINVPKTMESGTFITDKXVLDM | 164 |
| degenerate)-fP2A- | KAMDSKSNGAIAWSNQTSFTCQDIFKETNATYPSSDVPCDATLTEKSFETDM | |
| mbIL15-fT2A | NLNFQNLXVXXLRILLLKVAGFNLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHLRAKRSGSGEGRGS | |
| | LLTCGDVEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cα (murine, | XIQNPEPAVYQLKDPRSQDSTLCLFTDFDSQINVPKTMESGTFITDKXVLDM | 165 |
| degenerate)-fT2A- | KAMDSKSNGAIAWSNQTSFTCQDIFKETNATYPSSDVPCDATLTEKSFETDM | |
| mbIL15-fP2A | NLNFQNLXVXXLRILLLKVAGFNLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHLRAKRSGSGATNESL | |
| | LKQAGDVEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 166 |
| degenerate)-fP2A | HSGVXTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGP | |
| | X at position 57 is Ser or Cys | |
| Cα (murine, | XIQNPEPAVYQLKDPRSQDSTLCLFTDFDSQINVPKTMESGTFITDKXVLDM | 167 |
| degenerate)-fT2A- | KAMDSKSNGAIAWSNQTSFTCQDIFKETNATYPSSDVPCDATLTEKSFETDM | |
| mbIL15 | NLNFQNLXVXXLRILLLKVAGENLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHL | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 168 |
| degenerate)-fT2A | HSGVXTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGP | |
| | X at position 57 is Ser or Cys | |
| Cα (murine, | XIQNPEPAVYQLKDPRSQDSTLCLFTDFDSQINVPKTMESGTFITDKXVLDM | 169 |
| degenerate)-fP2A- | KAMDSKSNGAIAWSNQTSFTCQDIFKETNATYPSSDVPCDATLTEKSFETDM | |
| mbIL15 | NLNFQNLXVXXLRILLLKVAGENLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHL | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 170 |
| degenerate)-fP2A- | HSGVXTDPQAYKESNYSYCLSSRLRVSATFWHNPRNHFRCQVQFHGLSEEDK | |
| mbIL15-fT2A | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHLRAKRSGSGEGRGSLLTCGDVEENPGP | |
| | X at position 57 is Ser or Cys | |
| Cβ (murine, | EDLRNVTPPKVSLEEPSKAEIANKQKATLVCLARGFFPDHVELSWWVNGKEV | 171 |
| degenerate)-fT2A- | HSGVXTDPQAYKESNYSYCLSSRLRVSATFWHNPRNHFRCQVQFHGLSEEDK | |
| mbIL15-fP2A | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHLRAKRSGSGATNFSLLKQAGDVEENPGP | |
| | X at position 57 is Ser or Cys | |
| mbIL15-fP2A | MDWTWILFLVAAATRVHSNWVNVISDLKKIEDLIQSMHIDATLYTESDVHPS | 172 |
| | CKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGS | |
| | LQITCPPPMSVEHADIWVKSYSLYSRERYICNSGFKRKAGTSSLTECVLNKA | |
| | TNVAHWTTPSLKCIRDPALVHQRPAPPSTVTTAGVTPQPESLSPSGKEPAAS | |
| | SPSSNNTAATTAAIVPGSQLMPSKSPSTGTTEISSHESSHGTPSQTTAKNWE | |
| | LTASASHQPPGVYPQGHSDTTVAISTSTVLLCGLSAVSLLACYLKSRQTPPL | |
| | ASVEMEAMEALPVTWGTSSRDEDLENCSHHLRAKRSGSGATNFSLLKQAGDV | |
| | EENPGP | |
| mbIL15-fT2A | MDWTWILFLVAAATRVHSNWVNVISDLKKIEDLIQSMHIDATLYTESDVHPS | 173 |
| | CKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGS | |
| | LQITCPPPMSVEHADIWVKSYSLYSRERYICNSGFKRKAGTSSLTECVLNKA | |
| | TNVAHWTTPSLKCIRDPALVHQRPAPPSTVTTAGVTPQPESLSPSGKEPAAS | |
| | SPSSNNTAATTAAIVPGSQLMPSKSPSTGTTEISSHESSHGTPSQTTAKNWE | |
| | LTASASHQPPGVYPQGHSDTTVAISTSTVLLCGLSAVSLLACYLKSRQTPPL | |
| | ASVEMEAMEALPVTWGTSSRDEDLENCSHHLRAKRSGSGEGRGSLLTCGDVE | |
| | ENPGP | |

TABLE 10B
Exemplary amino acid sequences encoded by polycistronic expression cassettes

| | | SEQ ID |
|---|---|---|
| Description | Sequence | NO: |
| Cα (murine, cysteine- | NIQNPEPAVYQLKDPRSQDSTLCLFTDFDSQINVPKTMESGTFITDKCVLDM | 180 |
| and LIV-substituted)- | KAMDSKSNGAIAWSNQTSFTCQDIFKETNATYPSSDVPCDATLTEKSFETDM | |
| fP2A | NLNFQNLLVIVLRILLLKVAGENLLMTLRLWSSRAKRSGSGATNFSLLKQAG | |
| | DVEENPGP | |
| Cβ (murine, cysteine- | EDLRNVTPPKVSLEEPSKAEIANKQKATLVCLARGFFPDHVELSWWVNGKEV | 181 |
| substituted)-fT2A- | HSGVCTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHL | |
| Cα (murine, cysteine- | NIQNPEPAVYQLKDPRSQDSTLCLFTDFDSQINVPKTMESGTFITDKCVLDM | 182 |
| and LIV-substituted)- | KAMDSKSNGAIAWSNQTSFTCQDIFKETNATYPSSDVPCDATLTEKSFETDM | |
| fT2A | NLNFQNLLVIVLRILLLKVAGENLLMTLRLWSSRAKRSGSGEGRGSLLTCGD | |
| | VEENPGP | |
| Cβ (murine, cysteine- | EDLRNVTPPKVSLFEPSKAEIANKQKATLVCLARGFFPDHVELSWWVNGKEV | 183 |
| substituted)-fP2A- | HSGVCTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHL | |
| Cα (murine, cysteine- | NIQNPEPAVYQLKDPRSQDSTLCLFTDFDSQINVPKTMESGTFITDKCVLDM | 184 |
| and LIV-substituted)- | KAMDSKSNGAIAWSNQTSFTCQDIFKETNATYPSSDVPCDATLTEKSFETDM | |
| fP2A-mbIL15-fT2A | NLNFQNLLVIVLRILLLKVAGENLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHLRAKRSGSGEGRGS | |
| | LLTCGDVEENPGP | |
| Cα (murine, cysteine- | NIQNPEPAVYQLKDPRSQDSTLCLFTDFDSQINVPKTMESGTFITDKCVLDM | 185 |
| and LIV-substituted)- | KAMDSKSNGAIAWSNQTSFTCQDIFKETNATYPSSDVPCDATLTEKSFETDM | |
| fT2A-mbIL15-fP2A | NLNFQNLLVIVLRILLLKVAGENLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHLRAKRSGSGATNESL | |
| | LKQAGDVEENPGP | |
| Cβ (murine, cysteine- | EDLRNVTPPKVSLFEPSKAEIANKQKATLVCLARGFFPDHVELSWWVNGKEV | 186 |
| substituted)-fP2A | HSGVCTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGP | |
| Cα (murine, cysteine- | NIQNPEPAVYQLKDPRSQDSTLCLFTDFDSQINVPKTMESGTFITDKCVLDM | 187 |
| and LIV-substituted)- | KAMDSKSNGAIAWSNQTSFTCQDIFKETNATYPSSDVPCDATLTEKSFETDM | |
| fT2A-mbIL15 | NLNFQNLLVIVLRILLLKVAGENLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHL | |
| Cβ (murine, cysteine- | EDLRNVTPPKVSLFEPSKAEIANKQKATLVCLARGFFPDHVELSWWVNGKEV | 188 |
| substituted)-fT2A | HSGVCTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGP | |
| Cα (murine, cysteine- | NIQNPEPAVYQLKDPRSQDSTLCLFTDFDSQINVPKTMESGTFITDKCVLDM | 189 |
| and LIV-substituted)- | KAMDSKSNGAIAWSNQTSFTCQDIFKETNATYPSSDVPCDATLTEKSFETDM | |
| fP2A-mbIL15 | NLNFQNLLVIVLRILLLKVAGENLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHL | |
| Cβ (murine, cysteine- | EDLRNVTPPKVSLFEPSKAEIANKQKATLVCLARGFFPDHVELSWWVNGKEV | 190 |
| substituted)-fP2A- | HSGVCTDPQAYKESNYSYCLSSRLRVSATFWHNPRNHFRCQVQFHGLSEEDK | |
| mbIL15-fT2A | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHLRAKRSGSGEGRGSLLTCGDVEENPGP | |
| Cβ (murine, cysteine- | EDLRNVTPPKVSLFEPSKAEIANKQKATLVCLARGFFPDHVELSWWVNGKEV | 191 |
| substituted)-fT2A- | HSGVCTDPQAYKESNYSYCLSSRLRVSATFWHNPRNHFRCQVQFHGLSEEDK | |
| mbIL15-fP2A | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHLRAKRSGSGATNFSLLKQAGDVEENPGP | |

TABLE 10C
Exemplary amino acid sequences encoded by polycistronic expression cassettes

| | | SEQ ID |
|---|---|---|
| Description | Sequence | NO: |
| Cα (murine, LIV | NIQNPEPAVYQLKDPRSQDSTLCLFTDFDSQINVPKTMESGTFITDKTVLDM | 210 |
| substituted)-fP2A | KAMDSKSNGAIAWSNQTSFTCQDIFKETNATYPSSDVPCDATLTEKSFETDM | |
| | NLNFQNLLVIVLRILLLKVAGENLLMTLRLWSSRAKRSGSGATNFSLLKQAG | |
| | DVEENPGP | |
| Cα (murine, LIV | NIQNPEPAVYQLKDPRSQDSTLCLFTDFDSQINVPKTMESGTFITDKTVLDM | 212 |
| substituted)-fT2A | KAMDSKSNGAIAWSNQTSFTCQDIFKETNATYPSSDVPCDATLTEKSFETDM | |
| | NLNFQNLLVIVLRILLLKVAGENLLMTLRLWSSRAKRSGSGEGRGSLLTCGD | |
| | VEENPGP | |
| Cα (murine, LIV | NIQNPEPAVYQLKDPRSQDSTLCLFTDFDSQINVPKTMESGTFITDKTVLDM | 214 |
| substituted)-fP2A- | KAMDSKSNGAIAWSNQTSFTCQDIFKETNATYPSSDVPCDATLTEKSFETDM | |
| mbIL15-fT2A | NLNFQNLLVIVLRILLLKVAGENLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHLRAKRSGSGEGRGS | |
| | LLTCGDVEENPGP | |
| Cα (murine, LIV | NIQNPEPAVYQLKDPRSQDSTLCLFTDFDSQINVPKTMESGTFITDKTVLDM | 215 |
| substituted)-fT2A- | KAMDSKSNGAIAWSNQTSFTCQDIFKETNATYPSSDVPCDATLTEKSFETDM | |
| mbIL15-fP2A | NLNFQNLLVIVLRILLLKVAGENLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHLRAKRSGSGATNFSL | |
| | LKQAGDVEENPGP | |
| Cα (murine, LIV | NIQNPEPAVYQLKDPRSQDSTLCLFTDFDSQINVPKTMESGTFITDKTVLDM | 217 |
| substituted)-fT2A- | KAMDSKSNGAIAWSNQTSFTCQDIFKETNATYPSSDVPCDATLTEKSFETDM | |
| mbIL15 | NLNFQNLLVIVLRILLLKVAGENLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHL | |
| Cα (murine, LIV | NIQNPEPAVYQLKDPRSQDSTLCLFTDFDSQINVPKTMESGTFITDKTVLDM | 219 |
| substituted)-fP2A- | KAMDSKSNGAIAWSNQTSFTCQDIFKETNATYPSSDVPCDATLTEKSFETDM | |
| mbIL15 | NLNFQNLLVIVLRILLLKVAGENLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHL | |

Tables 11A, B and C below provide exemplary polynucleotide sequences for use in constructing vectors of the present disclosure. As shown in Tables 11A, B and C, vectors of the present disclosure can include one or more of the following sequences: (1) an “AP” sequence which encodes (i) a Cα sequence disclosed herein and (ii) a P2A element sequence disclosed herein; (2) a “BT” sequence which encodes (i) a Cβ sequence disclosed herein and (ii) a T2A element sequence disclosed herein; (3) a “BT15” sequence which encodes (i) a Cβ sequence disclosed herein, (ii) a T2A element sequence disclosed herein, and (iii) a mbIL15 sequence disclosed herein; (4) an “AT” sequence which encodes (i) a Cα sequence disclosed herein and (ii) a T2A element sequence disclosed herein; (5) a “BP” sequence which encodes (i) a Cβ sequence disclosed herein and (ii) a P2A element sequence disclosed herein; (6) a “BP15” sequence which encodes (i) a Cβ sequence disclosed herein, (ii) a P2A element sequence disclosed herein, and (iii) a mbIL15 sequence disclosed herein; (7) an “AP15” sequence which encodes (i) a Cα sequence disclosed herein, (ii) a P2A element sequence disclosed herein, and (iii) a mbIL15 sequence disclosed herein; (8) a “15T” sequence which encodes (i) a mbIL15 sequence disclosed herein and (ii) a T2A element sequence disclosed herein; (9) an “AP15T” sequence which encodes (i) a Cα sequence disclosed herein, (ii) a P2A element sequence disclosed herein, (iii) a mbIL15 sequence disclosed herein, and (iv) a T2A element sequence disclosed herein (10) an “AT15” sequence which encodes (i) a Cα sequence disclosed herein, (ii) a T2A element sequence disclosed herein, and (iii) a mbIL15 sequence disclosed herein; (11) a “15P” sequence which encodes (i) a mbIL15 sequence disclosed herein, and (ii) a P2A element sequence disclosed herein; (12) an “AT15P” sequence which encodes (i) a Cα sequence disclosed herein, (ii) a T2A element sequence disclosed herein, (iii) a mbIL15 sequence disclosed herein, and (iv) a P2A element sequence disclosed herein; (13) an “BP15T” sequence which encodes (i) a Cβ sequence disclosed herein, (ii) a P2A element sequence disclosed herein, (iii) a mbIL15 sequence disclosed herein, and (iv) a T2A element sequence disclosed herein; (14) an “BT15P” sequence which encodes (i) a Cβ sequence disclosed herein, (ii) a T2A element sequence disclosed herein, (iii) a mbIL15 sequence disclosed herein, and (iv) a P2A element sequence disclosed herein.

The nucleotide sequences provided herein (and their corresponding amino acid sequences) may be used in any appropriate combination. An “appropriate combination” is a combination where desired molecular function(s) are provided by one or more of the sequences disclosed herein. For example, in general, any 2A element sequence provided herein can provide the function of ribosome skipping (via the 2A element) and, optionally, furin-mediated cleavage (via the furin recognition site). Thus, an “AT” sequence in a vector of the present disclosure could, in alternative embodiments, be replaced by an “AP” sequence of the present disclosure. Similarly, “AE” and “AF” sequences, comprising Cα region sequences and E2A or F2A element sequences can also be used. “BT,” “BP,” “BE,” and “BF” sequences comprising Cβ region sequences and 2A element sequences are all also interchangeable. “15T,” “15P,” “15E,” and “15F” sequences comprising mbIL15 sequences and 2A element sequences are all also interchangeable. Additionally, any combination of TCRα, TCRβ, and mbIL15 sequences may appear from 5′ to 3′ on a vector of the present disclosure in any order and may be separated by sequences which provide appropriate 2A element sequence function (e.g., ribosome skipping, furin cleavage).

Accordingly, sequences of the present disclosure provide ribosome skipping, furin recognition, TCRα function, TCRβ function, and mbIL15 function in any appropriate combination or 5′ to 3′ order.

TABLE 11A
Exemplary polynucleotide sequences for use in polycistronic expression

| | | SEQ ID |
|---|---|---|
| Description | Sequence | NO: |
| AP nucleotide | NNNATCCAGAATCCCGAGCCTGCGGTGTACCAGCTGAAGGACCCCCGCTCT | 230 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCA | |
| | NNN at positions 1-3 make up a | |
| | codon that encodes Asn, Asp, His, or Tyr; | |
| | NNN at positions 142-144 make up a | |
| | codon that encodes Thr or Cys; | |
| | NNN at positions 334-336 make up a | |
| | codon that encodes Ser, Ala, Val, Leu, | |
| | Ile, Pro, Phe, Met, or Trp; | |
| | NNN at positions 340-342 make up a | |
| | codon that encodes Met, Ala, Val, Leu, | |
| | Ile, Pro, Phe, or Trp; | |
| | NNN at positions 343-345 make up a | |
| | codon that encodes Gly, Ala, Val, Leu, | |
| | Ile, Pro, Phe, Met, or Trp | |
| BT nucleotide | GAGGACCTGAGGAACGTGACCCCACCTAAAGTGAGCCTGTTCGAGCCATCC | 231 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCT | |
| | NNN at positions 169-171 make up a | |
| | codon that encodes Ser or Cys | |
| BT15 nucleotide | GAGGACCTGAGGAACGTGACCCCACCTAAAGTGAGCCTGTTCGAGCCATCC | 232 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTG | |
| | NNN at positions 169-171 make up a | |
| | codon that encodes Ser or Cys | |
| AT nucleotide | NNNATCCAGAATCCCGAGCCTGCGGTGTACCAGCTGAAGGACCCCCGCTCT | 233 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCT | |
| | NNN at positions 1-3 make up a | |
| | codon that encodes Asn, Asp, His, or Tyr; | |
| | NNN at positions 142-144 make up a | |
| | codon that encodes Thr or Cys; | |
| | NNN at positions 334-336 make up a | |
| | codon that encodes Ser, Ala, Val, Leu, | |
| | Ile, Pro, Phe, Met, or Trp; | |
| | NNN at positions 340-342 make up a | |
| | codon that encodes Met, Ala, Val, Leu, | |
| | Ile, Pro, Phe, or Trp; | |
| | NNN at positions 343-345 make up a | |
| | codon that encodes Gly, Ala, Val, Leu, | |
| | Ile, Pro, Phe, Met, or Trp | |
| BP nucleotide | GAGGACCTGAGGAACGTGACCCCACCTAAAGTGAGCCTGTTCGAGCCATCC | 234 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCA | |
| | NNN at positions 169-171 make up a | |
| | codon that encodes Ser or Cys | |
| BP15 nucleotide | GAGGACCTGAGGAACGTGACCCCACCTAAAGTGAGCCTGTTCGAGCCATCC | 235 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| | NNN at positions 169-171 make up a | |
| | codon that encodes Ser or Cys | |
| AP15 nucleotide | NNNATCCAGAATCCCGAGCCTGCGGTGTACCAGCTGAAGGACCCCCGCTCT | 236 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTG | |
| | NNN at positions 1-3 make up a | |
| | codon that encodes Asn, Asp, His, or Tyr; | |
| | NNN at positions 142-144 make up a | |
| | codon that encodes Thr or Cys; | |
| | NNN at positions 334-336 make up a | |
| | codon that encodes Ser, Ala, Val, Leu, | |
| | Ile, Pro, Phe, Met, or Trp; | |
| | NNN at positions 340-342 make up a | |
| | codon that encodes Met, Ala, Val, Leu, | |
| | Ile, Pro, Phe, or Trp; | |
| | NNN at positions 343-345 make up a | |
| | codon that encodes Gly, Ala, Val, Leu, | |
| | Ile, Pro, Phe, Met, or Trp | |
| 15T nucleotide | ATGGATTGGACCTGGATTCTGTTTCTGGTGGCCGCTGCCACAAGAGTGCAC | 237 |
| sequence | AGCAACTGGGTGAATGTGATCAGCGACCTGAAGAAGATCGAGGATCTGATC | |
| | CAGAGCATGCACATTGATGCCACCCTGTACACAGAATCTGATGTGCACCCT | |
| | AGCTGTAAAGTGACCGCCATGAAGTGTTTTCTGCTGGAGCTGCAGGTGATT | |
| | TCTCTGGAAAGCGGAGATGCCTCTATCCACGACACAGTGGAGAATCTGATC | |
| | ATCCTGGCCAACAATAGCCTGAGCAGCAATGGCAATGTGACAGAGTCTGGC | |
| | TGTAAGGAGTGTGAGGAGCTGGAGGAGAAGAACATCAAGGAGTTTCTGCAG | |
| | AGCTTTGTGCACATCGTGCAGATGTTCATCAATACAAGCTCTGGCGGAGGA | |
| | TCTGGAGGAGGCGGATCTGGAGGAGGAGGCAGTGGAGGCGGAGGATCTGGC | |
| | GGAGGATCTCTGCAGATTACATGCCCTCCTCCAATGTCTGTGGAGCACGCC | |
| | GATATTTGGGTGAAGTCCTACAGCCTGTACAGCAGAGAGAGATACATCTGC | |
| | AACAGCGGCTTTAAGAGAAAGGCCGGCACCTCTTCTCTGACAGAGTGCGTG | |
| | CTGAATAAGGCCACAAATGTGGCCCACTGGACAACACCTAGCCTGAAGTGC | |
| | ATTAGAGATCCTGCCCTGGTCCACCAGAGGCCTGCCCCTCCATCTACAGTG | |
| | ACAACAGCCGGAGTGACACCTCAGCCTGAATCTCTGAGCCCTTCTGGAAAA | |
| | GAACCTGCCGCCAGCTCTCCTAGCTCTAATAATACCGCCGCCACAACAGCC | |
| | GCCATTGTGCCTGGATCTCAGCTGATGCCTAGCAAGTCTCCTAGCACAGGC | |
| | ACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGA | |
| | GTGTATCCTCAGGGCCACTCTGATACAACAGTGGCCATCAGCACATCTACA | |
| | GTGCTGCTGTGTGGACTGTCTGCCGTGTCTCTGCTGGCCTGTTACCTGAAG | |
| | TCTAGACAGACACCTCCTCTGGCCTCTGTGGAGATGGAGGCCATGGAAGCC | |
| | CTGCCTGTGACATGGGGAACAAGCAGCAGAGATGAAGACCTGGAGAATTGT | |
| | TCTCACCACCTGCGGGCGAAACGCTCTGGAAGCGGAGAGGGCAGAGGAAGT | |
| | CTTCTAACATGCGGTGACGTGGAGGAGAATCCCGGCCCT | |
| AP15T nucleotide | NNNATCCAGAATCCCGAGCCTGCGGTGTACCAGCTGAAGGACCCCCGCTCT | 238 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTGCGGGCG | |
| | AAACGCTCTGGAAGCGGAGAGGGCAGAGGAAGTCTTCTAACATGCGGTGAC | |
| | GTGGAGGAGAATCCCGGCCCT | |
| | NNN at positions 1-3 make up a | |
| | codon that encodes Asn, Asp, His, or Tyr; | |
| | NNN at positions 142-144 make up a | |
| | codon that encodes Thr or Cys; | |
| | NNN at positions 334-336 make up a | |
| | codon that encodes Ser, Ala, Val, Leu, | |
| | Ile, Pro, Phe, Met, or Trp; | |
| | NNN at positions 340-342 make up a | |
| | codon that encodes Met, Ala, Val, Leu, | |
| | Ile, Pro, Phe, or Trp; | |
| | NNN at positions 343-345 make up a | |
| | codon that encodes Gly, Ala, Val, Leu, | |
| | Ile, Pro, Phe, Met, or Trp | |
| AT15 nucleotide | NNNATCCAGAATCCCGAGCCTGCGGTGTACCAGCTGAAGGACCCCCGCTCT | 239 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTG | |
| | NNN at positions 1-3 make up a | |
| | codon that encodes Asn, Asp, His, or Tyr; | |
| | NNN at positions 142-144 make up a | |
| | codon that encodes Thr or Cys; | |
| | NNN at positions 334-336 make up a | |
| | codon that encodes Ser, Ala, Val, Leu, | |
| | Ile, Pro, Phe, Met, or Trp; | |
| | NNN at positions 340-342 make up a | |
| | codon that encodes Met, Ala, Val, Leu, | |
| | Ile, Pro, Phe, or Trp; | |
| | NNN at positions 343-345 make up a | |
| | codon that encodes Gly, Ala, Val, Leu, | |
| | Ile, Pro, Phe, Met, or Trp | |
| 15P nucleotide | ATGGATTGGACCTGGATTCTGTTTCTGGTGGCCGCTGCCACAAGAGTGCAC | 240 |
| sequence | AGCAACTGGGTGAATGTGATCAGCGACCTGAAGAAGATCGAGGATCTGATC | |
| | CAGAGCATGCACATTGATGCCACCCTGTACACAGAATCTGATGTGCACCCT | |
| | AGCTGTAAAGTGACCGCCATGAAGTGTTTTCTGCTGGAGCTGCAGGTGATT | |
| | TCTCTGGAAAGCGGAGATGCCTCTATCCACGACACAGTGGAGAATCTGATC | |
| | ATCCTGGCCAACAATAGCCTGAGCAGCAATGGCAATGTGACAGAGTCTGGC | |
| | TGTAAGGAGTGTGAGGAGCTGGAGGAGAAGAACATCAAGGAGTTTCTGCAG | |
| | AGCTTTGTGCACATCGTGCAGATGTTCATCAATACAAGCTCTGGCGGAGGA | |
| | TCTGGAGGAGGCGGATCTGGAGGAGGAGGCAGTGGAGGCGGAGGATCTGGC | |
| | GGAGGATCTCTGCAGATTACATGCCCTCCTCCAATGTCTGTGGAGCACGCC | |
| | GATATTTGGGTGAAGTCCTACAGCCTGTACAGCAGAGAGAGATACATCTGC | |
| | AACAGCGGCTTTAAGAGAAAGGCCGGCACCTCTTCTCTGACAGAGTGCGTG | |
| | CTGAATAAGGCCACAAATGTGGCCCACTGGACAACACCTAGCCTGAAGTGC | |
| | ATTAGAGATCCTGCCCTGGTCCACCAGAGGCCTGCCCCTCCATCTACAGTG | |
| | ACAACAGCCGGAGTGACACCTCAGCCTGAATCTCTGAGCCCTTCTGGAAAA | |
| | GAACCTGCCGCCAGCTCTCCTAGCTCTAATAATACCGCCGCCACAACAGCC | |
| | GCCATTGTGCCTGGATCTCAGCTGATGCCTAGCAAGTCTCCTAGCACAGGC | |
| | ACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGA | |
| | GTGTATCCTCAGGGCCACTCTGATACAACAGTGGCCATCAGCACATCTACA | |
| | GTGCTGCTGTGTGGACTGTCTGCCGTGTCTCTGCTGGCCTGTTACCTGAAG | |
| | TCTAGACAGACACCTCCTCTGGCCTCTGTGGAGATGGAGGCCATGGAAGCC | |
| | CTGCCTGTGACATGGGGAACAAGCAGCAGAGATGAAGACCTGGAGAATTGT | |
| | TCTCACCACCTGCGGGCGAAACGCTCTGGAAGCGGAGCGACCAATTTCAGC | |
| | CTGCTGAAGCAGGCGGGCGATGTGGAGGAGAACCCTGGCCCA | |
| AT15P nucleotide | NNNATCCAGAATCCCGAGCCTGCGGTGTACCAGCTGAAGGACCCCCGCTCT | 241 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTGCGGGCGAAA | |
| | CGCTCTGGAAGCGGAGCGACCAATTTCAGCCTGCTGAAGCAGGCGGGCGAT | |
| | GTGGAGGAGAACCCTGGCCCA | |
| | NNN at positions 1-3 make up a | |
| | codon that encodes Asn, Asp, His, or Tyr; | |
| | NNN at positions 142-144 make up a | |
| | codon that encodes Thr or Cys; | |
| | NNN at positions 334-336 make up a | |
| | codon that encodes Ser, Ala, Val, Leu, | |
| | Ile, Pro, Phe, Met, or Trp; | |
| | NNN at positions 340-342 make up a | |
| | codon that encodes Met, Ala, Val, Leu, | |
| | Ile, Pro, Phe, or Trp; | |
| | NNN at positions 343-345 make up a | |
| | codon that encodes Gly, Ala, Val, Leu, | |
| | Ile, Pro, Phe, Met, or Trp | |
| BP15T nucleotide | GAGGACCTGAGGAACGTGACCCCACCTAAAGTGAGCCTGTTCGAGCCATCC | 242 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| | CGGGCGAAACGCTCTGGAAGCGGAGAGGGCAGAGGAAGTCTTCTAACATGC | |
| | GGTGACGTGGAGGAGAATCCCGGCCCT | |
| | NNN at positions 169-171 make up a | |
| | codon that encodes Ser or Cys | |
| BT15P nucleotide | GAGGACCTGAGGAACGTGACCCCACCTAAAGTGAGCCTGTTCGAGCCATCC | 243 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTGCGG | |
| | GCGAAACGCTCTGGAAGCGGAGCGACCAATTTCAGCCTGCTGAAGCAGGCG | |
| | GGCGATGTGGAGGAGAACCCTGGCCCA | |
| | NNN at positions 169-171 make up a | |
| | codon that encodes Ser or Cys | |

TABLE 11B
Exemplary polynucleotide sequences for use in polycistronic expression
cassette.

| | | SEQ ID |
|---|---|---|
| Description | Sequence | NO: |
| AP nucleotide | AACATCCAGAATCCCGAGCCTGCGGTGTACCAGCTGAAGGACCCCCGCTCT | 250 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCA | |
| BT nucleotide | GAGGACCTGAGGAACGTGACCCCACCTAAAGTGAGCCTGTTCGAGCCATCC | 251 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCT | |
| BT15 nucleotide | GAGGACCTGAGGAACGTGACCCCACCTAAAGTGAGCCTGTTCGAGCCATCC | 252 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTG | |
| AT nucleotide | AACATCCAGAATCCCGAGCCTGCGGTGTACCAGCTGAAGGACCCCCGCTCT | 253 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCT | |
| BP nucleotide | GAGGACCTGAGGAACGTGACCCCACCTAAAGTGAGCCTGTTCGAGCCATCC | 254 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCA | |
| BP15 nucleotide | GAGGACCTGAGGAACGTGACCCCACCTAAAGTGAGCCTGTTCGAGCCATCC | 255 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| AP15 nucleotide | AACATCCAGAATCCCGAGCCTGCGGTGTACCAGCTGAAGGACCCCCGCTCT | 256 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTG | |
| AP15T nucleotide | AACATCCAGAATCCCGAGCCTGCGGTGTACCAGCTGAAGGACCCCCGCTCT | 258 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTGCGGGCG | |
| | AAACGCTCTGGAAGCGGAGAGGGCAGAGGAAGTCTTCTAACATGCGGTGAC | |
| | GTGGAGGAGAATCCCGGCCCT | |
| AT15 nucleotide | AACATCCAGAATCCCGAGCCTGCGGTGTACCAGCTGAAGGACCCCCGCTCT | 259 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTG | |
| AT15P nucleotide | AACATCCAGAATCCCGAGCCTGCGGTGTACCAGCTGAAGGACCCCCGCTCT | 261 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTGCGGGCGAAA | |
| | CGCTCTGGAAGCGGAGCGACCAATTTCAGCCTGCTGAAGCAGGCGGGCGAT | |
| | GTGGAGGAGAACCCTGGCCCA | |
| BP15T nucleotide | GAGGACCTGAGGAACGTGACCCCACCTAAAGTGAGCCTGTTCGAGCCATCC | 262 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| | CGGGCGAAACGCTCTGGAAGCGGAGAGGGCAGAGGAAGTCTTCTAACATGC | |
| | GGTGACGTGGAGGAGAATCCCGGCCCT | |
| BT15P nucleotide | GAGGACCTGAGGAACGTGACCCCACCTAAAGTGAGCCTGTTCGAGCCATCC | 263 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTGCGG | |
| | GCGAAACGCTCTGGAAGCGGAGCGACCAATTTCAGCCTGCTGAAGCAGGCG | |
| | GGCGATGTGGAGGAGAACCCTGGCCCA | |

TABLE 11C
Exemplary polynucleotide sequences for use in polycistronic expression cassette.

| | | SEQ ID |
|---|---|---|
| Description | Sequence | NO: |
| AP nucleotide | AACATCCAGAATCCCGAGCCTGCGGTGTACCAGCTGAAGGACCCCCGCTCT | 270 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCA | |
| AT nucleotide | AACATCCAGAATCCCGAGCCTGCGGTGTACCAGCTGAAGGACCCCCGCTCT | 273 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCT | |
| AP15 nucleotide | AACATCCAGAATCCCGAGCCTGCGGTGTACCAGCTGAAGGACCCCCGCTCT | 276 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTG | |
| AP15T nucleotide | AACATCCAGAATCCCGAGCCTGCGGTGTACCAGCTGAAGGACCCCCGCTCT | 278 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTGCGGGCG | |
| | AAACGCTCTGGAAGCGGAGAGGGCAGAGGAAGTCTTCTAACATGCGGTGAC | |
| | GTGGAGGAGAATCCCGGCCCT | |
| AT15 nucleotide | AACATCCAGAATCCCGAGCCTGCGGTGTACCAGCTGAAGGACCCCCGCTCT | 279 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTG | |
| AT15P nucleotide | AACATCCAGAATCCCGAGCCTGCGGTGTACCAGCTGAAGGACCCCCGCTCT | 281 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTGCGGGCGAAA | |
| | CGCTCTGGAAGCGGAGCGACCAATTTCAGCCTGCTGAAGCAGGCGGGCGAT | |
| | GTGGAGGAGAACCCTGGCCCA | |

### 5.7 Transposon and Transposase Systems

In some embodiments, transgenes of the recombinant vector are introduced into an immune effector cell via synthetic DNA transposable elements, e.g., a DNA transposon/transposase system, e.g., Sleeping Beauty (SB). SB belongs to the Tc1/mariner superfamily of DNA transposons. DNA transposons translocate from one DNA site to another in a simple, cut-and-paste manner. Transposition is a precise process in which a defined DNA segment is excised from one DNA molecule and moved to another site in the same or different DNA molecule or genome.

Exemplary DNA transposon/transposase systems include, but are not limited to, Sleeping Beauty (see, e.g., U.S. Pat. Nos. 6,489,458, 8,227,432, the contents of each of which are incorporated by reference in their entirety herein), piggyBac transposon system (see e.g., U.S. Pat. No. 9,228,180, Wilson et al, “PiggyBac Transposon-mediated Gene Transfer in Human Cells,” Molecular Therapy, 15:139-145 (2007), the contents of each of which are incorporated by reference in their entirety herein), piggyBac transposon system (see e.g., Mitra et al., “Functional characterization of piggyBac from the bat Myotis lucifugus unveils an active mammalian DNA transposon,” Proc. Natl. Acad. Sci USA 110:234-239 (2013), the contents of which are incorporated by reference in their entirety herein), TcBuster (see e.g., Woodard et al. “Comparative Analysis of the Recently Discovered hAT Transposon TcBuster in Human Cells,” PLOS ONE, 7(11): e42666 (November 2012), the contents of which are incorporated by reference in their entirety herein), and the Tol2 transposon system (see e.g., Kawakami, “Tol2: a versatile gene transfer vector in vertebrates,” Genome Biol. 2007; 8(Suppl 1): S7, the contents of each of which are incorporated by reference in their entirety herein). Additional exemplary transposon/transposase systems are provided in U.S. Pat. Nos. 7,148,203; 8,227,432; US20110117072; Mates et al., Nat Genet, 41(6):753-61 (2009); and Ivies et al., Cell, 91(4):501-10, (1997), the contents of each of which are incorporated by reference in their entirety herein).

In some embodiments, the transgenes described herein are introduced into an immune effector cell via the SB transposon/transposase system. The SB transposon system comprises a SB a transposase and SB transposon(s). The SB transposon system can comprise a naturally occurring SB transposase or a derivative, variant, and/or fragment that retains activity, and a naturally occurring SB transposon, or a derivative, variant, and/or fragment that retains activity. An exemplary SB system is described in, Hackett et al., “A Transposon and Transposase System for Human Application,” Mol Ther 18:674-83, (2010), the entire contents of which are incorporated by reference herein.

In some embodiments, the vector comprises a Left inverted terminal repeat (ITR), i.e., an ITR that is 5′ to an expression cassette, and a Right ITR, i.e., an ITR that is 3′ to an expression cassette. The Left ITR and Right ITR flank the polycistronic expression cassette of the vector. In some embodiments, the Left ITR is in reverse orientation relative to the polycistronic expression cassette, and the Right ITR is in the same orientation relative to the polycistronic expression cassette. In some embodiments, the Right ITR is in reverse orientation relative to the polycistronic expression cassette, and the Left ITR is in the same orientation relative to the polycistronic expression cassette.

In some embodiments, the Left ITR and the Right ITR are ITRs of a DNA transposon selected from the group consisting of a Sleeping Beauty transposon, a piggyBac transposon, TcBuster transposon, and a Tol2 transposon. In some embodiments, the Left ITR and the Right ITR are ITRs of the Sleeping Beauty DNA transposon.

In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 290 or 291. In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 290. In some embodiments, the Left ITR comprises the polynucleotide sequence of SEQ ID NO: 290. In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 291. In some embodiments, the Left ITR comprises the polynucleotide sequence of SEQ ID NO: 291. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 292, 293, or 294. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 292. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 293. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 294. In some embodiments, the Right ITR comprises the polynucleotide sequence of SEQ ID NO: 292. In some embodiments, the Right ITR comprises the polynucleotide sequence of SEQ ID NO: 293. In some embodiments, the Right ITR comprises the polynucleotide sequence of SEQ ID NO: 294.

The polynucleotide sequences of exemplary SB ITRs are provided in Table 12, herein.

TABLE 12
Polynucleotide sequence of exemplary SB ITRs.

| Description | Polynucleotide Sequence | SEQ ID NO |
|---|---|---|
| Left ITR-A | AGTTGAAGTCGGAAGTTTACATACACTTAAGTTGGAGTCATTAAAA | 290 |
| | CTCGTTTTTCAACTACTCCACAAATTTCTTGTTAACAAACAATAGT | |
| | TTTGGCAAGTCAGTTAGGACATCTACTTTGTGCATGACACAAGTCA | |
| | TTTTTCCAACAATTGTTTACAGACAGATTATTTCACTTATAATTCA | |
| | CTGTATCACAATTCCAGTGGGTCAGAAGTTTACATACACTAA | |
| Left ITR-A2 | TACAGTTGAAGTCGGAAGTTTACATACACTTAAGTTGGAGTCATTA | 295 |
| | AAACTCGTTTTTCAACTACTCCACAAATTTCTTGTTAACAAACAAT | |
| | AGTTTTGGCAAGTCAGTTAGGACATCTACTTTGTGCATGACACAAG | |
| | TCATTTTTCCAACAATTGTTTACAGACAGATTATTTCACTTATAAT | |
| | TCACTGTATCACAATTCCAGTGGGTCAGAAGTTTACATACACTAA | |
| Left ITR-B | ATATCAATTGAGTTGAAGTCGGAAGTTTACATACACTTAAGTTGGA | 291 |
| | GTCATTAAAACTCGTTTTTCAACTACACCACAAATTTCTTGTTAAC | |
| | AAACAATAGTTTTGGCAAGTCAGTTAGGACATCTACTTTGTGCATG | |
| | ACACAAGTCATTTTTCCAACAATTGTTTACAGACAGATTATTTCAC | |
| | TTATAATTCACTGTATCACAATTCCAGTGGGTCAGAAGTTTACATA | |
| | CACTAACAATTGATAT | |
| Right ITR-A | TTGAGTGTATGTAAACTTCTGACCCACTGGGAATGTGATGAAAGAA | 292 |
| | ATAAAAGCTGAAATGAATCATTCTCTCTACTATTATTCTGATATTT | |
| | CACATTCTTAAAATAAAGTGGTGATCCTAACTGACCTAAGACAGGG | |
| | AATTTTTACTAGGATTAAATGTCAGGAATTGTGAAAAAGTGAGTTT | |
| | AAATGTATTTGGCTAAGGTGTATGTAAACTTCCGACTTCAACTG | |
| Right ITR-A2 | TTGAGTGTATGTAAACTTCTGACCCACTGGGAATGTGATGAAAGAA | 296 |
| | ATAAAAGCTGAAATGAATCATTCTCTCTACTATTATTCTGATATTT | |
| | CACATTCTTAAAATAAAGTGGTGATCCTAACTGACCTAAGACAGGG | |
| | AATTTTTACTAGGATTAAATGTCAGGAATTGTGAAAAAGTGAGTTT | |
| | AAATGTATTTGGCTAAGGTGTATGTAAACTTCCGACTTCAACTGTA | |
| Right ITR-B | TTGAGTGTATGTTAACTTCTGACCCACTGGGAATGTGATGAAAGAA | 293 |
| | ATAAAAGCTGAAATGAATCATTCTCTCTACTATTATTCTGATATTT | |
| | CACATTCTTAAAATAAAGTGGTGATCCTAACTGACCTTAAGACAGG | |
| | GAATCTTTACTCGGATTAAATGTCAGGAATTGTGAAAAAGTGAGTT | |
| | TAAATGTATTTGGCTAAGGTGTATGTAAACTTCCGACTTCAACT | |
| Right ITR-B2 | TTGAGTGTATGTTAACTTCTGACCCACTGGGAATGTGATGAAAGAA | 297 |
| | ATAAAAGCTGAAATGAATCATTCTCTCTACTATTATTCTGATATTT | |
| | CACATTCTTAAAATAAAGTGGTGATCCTAACTGACCTTAAGACAGG | |
| | GAATCTTTACTCGGATTAAATGTCAGGAATTGTGAAAAAGTGAGTT | |
| | TAAATGTATTTGGCTAAGGTGTATGTAAACTTCCGACTTCAACTGT | |
| | A | |
| Right ITR-C | ATATCTCGAGTTGAGTGTATGTTAACTTCTGACCCACTGGGAATGT | 294 |
| | GATGAAAGAAATAAAAGCTGAAATGAATCATTCTCTCTACTATTAT | |
| | TCTGATATTTCACATTCTTAAAATAAAGTGGTGATCCTAACTGACC | |
| | TTAAGACAGGGAATCTTTACTCGGATTAAATGTCAGGAATTGTGAA | |
| | AAAGTGAGTTTAAATGTATTTGGCTAAGGTGTATGTAAACTTCCGA | |
| | CTTCAACTCTCGAGATAT | |

In some embodiments, the DNA transposase is a SB transposase. In some embodiments, the SB transposase is selected from the group consisting of SB11, SB100X, hSB110, and hSB81. In some embodiments, the SB transposase is SB11. Exemplary SB transposases are described in U.S. Pat. No. 9,840,696, US20160264949, U.S. Pat. No. 9,228,180, WO2019038197, U.S. Ser. No. 10/174,309, and U.S. Ser. No. 10/570,382, the full contents of each of which is incorporated by reference herein.

In some embodiments, the DNA transposase comprises an amino acid sequence at least 95%, 96%, 97%, 98%, 99% or 100% identical to the amino acid sequence of SEQ ID NO: 300. In some embodiments, the DNA transposase comprises the amino acid sequence of SEQ ID NO: 300. In some embodiments, the amino acid sequence of the DNA transposase consists of a sequence at least 95%, 96%, 97%, 98%, 99% or 100% identical to the amino acid sequence of SEQ ID NO: 300. In some embodiments, the amino acid sequence of the DNA transposase consists of the amino acid sequence of SEQ ID NO: 300.

In some embodiments, the DNA transposase comprises an amino acid sequence that lacks its N-terminal methionine. In some embodiments, the DNA transposase comprises an amino acid sequence at least 95%, 96%, 97%, 98%, 99% or 100% identical to the amino acid sequence of SEQ ID NO: 300 lacking its N-terminal methionine, i.e., amino acids 2-340 of SEQ ID NO:300. In some embodiments, the DNA transposase comprises the amino acid sequence of SEQ ID NO: 300 lacking its N-terminal methionine, i.e., amino acids 2-340 of SEQ ID NO:300. In some embodiments, the amino acid sequence of the DNA transposase consists of a sequence at least 95%, 96%, 97%, 98%, 99% or 100% identical to the amino acid sequence of SEQ ID NO: 300 lacking its N-terminal methionine, i.e., amino acids 2-340 of SEQ ID NO:300. In some embodiments, the amino acid sequence of the DNA transposase consists of the amino acid sequence of SEQ ID NO: 300 lacking its N-terminal methionine, i.e., amino acids 2-340 of SEQ ID NO:300.

In some embodiments, the DNA transposase is encoded by a polynucleotide sequence at least 75%, 80%, 85%, 90°/a, 95%, 96%, 97%, 98%, 99%, or 100% identical to the polynucleotide sequence of SEQ ID NO: 301. In some embodiments, the DNA transposase is encoded by the polynucleotide sequence of SEQ ID NO: 301.

In some embodiments, the DNA transposase is encoded by a polynucleotide that is introduced into a cell. In some embodiments, the polynucleotide encoding the DNA transposase is a DNA vector. In some embodiments, the polynucleotide encoding the DNA transposase is an RNA vector. In some embodiments, the DNA transposase is encoded on a first vector and the transgenes are encoded on a second vector. In some embodiments, the DNA transposase is directly introduced to a population of cells as a polypeptide.

The amino acid and polynucleotide sequence of an exemplary SB transposase is provided in Table 13, herein.

TABLE 13
Amino acid and polynucleotide sequence of an exemplary SB transposase.

| Description | Sequence | SEQ ID NO |
|---|---|---|
| SB11 (exemplary | MGKSKEISQDLRKKIVDLHKSGSSLGAISKRLKVPRSSVQTIVRKYKH | 300 |
| amino acid | HGTTQPSYRSGRRRVLSPRDERTLVRKVQINPRTTAKDLVKMLEETGT | |
| sequence) | KVSISTVKRVLYRHNLKGRSARKKPLLQNRHKKARLRFARAHGDKDRT | |
| | FWRNVLWSDETKIELFGHNDHRYVWRKKGEACKPKNTIPTVKHGGGSI | |
| | MLWGCFAAGGTGALHKIDGIMRKENYVDILKQHLKTSVRKLKLGRKWV | |
| | FQQDNDPKHTSKHVRKWLKDNKVKVLEWPSQSPDLNPIENLWAELKKR | |
| | VRARRPTNLTQLHQLCQEEWAKIHPTYCGKLVEGYPKRLTQVKQFKGN | |
| | ATKY | |
| SB11 (exemplary | ATGGGAAAATCAAAAGAAATCAGCCAAGACCTCAGAAAAAAAATTGTA | 301 |
| nucleotide | GACCTCCACAAGTCTGGTTCATCCTTGGGAGCAATTTCCAAACGCCTG | |
| sequence) | AAAGTACCACGTTCATCTGTACAAACAATAGTACGCAAGTATAAACAC | |
| | CATGGGACCACGCAGCCGTCATACCGCTCAGGAAGGAGACGCGTTCTG | |
| | TCTCCTAGAGATGAACGTACTTTGGTGCGAAAAGTGCAAATCAATCCC | |
| | AGAACAACAGCAAAGGACCTTGTGAAGATGCTGGAGGAAACAGGTACA | |
| | AAAGTATCTATATCCACAGTAAAACGAGTCCTATATCGACATAACCTG | |
| | AAAGGCCGCTCAGCAAGGAAGAAGCCACTGCTCCAAAACCGACATAAG | |
| | AAAGCCAGACTACGGTTTGCAAGAGCACATGGGGACAAAGATCGTACT | |
| | TTTTGGAGAAATGTCCTCTGGTCTGATGAAACAAAAATAGAACTGTTT | |
| | GGCCATAATGACCATCGTTATGTTTGGAGGAAGAAGGGGGAGGCTTGC | |
| | AAGCCGAAGAACACCATCCCAACCGTGAAGCACGGGGGTGGCAGCATC | |
| | ATGTTGTGGGGGTGCTTTGCTGCAGGAGGGACTGGTGCACTTCACAAA | |
| | ATAGATGGCATCATGAGGAAGGAAAATTATGTGGATATATTGAAGCAA | |
| | CATCTCAAGACATCAGTCAGGAAGTTAAAGCTTGGTCGCAAATGGGTC | |
| | TTCCAACAAGACAATGACCCCAAGCATACTTCCAAACACGTGAGAAAA | |
| | TGGCTTAAGGACAACAAAGTCAAGGTATTGGAGTGGCCATCACAAAGC | |
| | CCTGACCTCAATCCTATAGAAAATTTGTGGGCAGAACTGAAAAAGCGT | |
| | GTGCGAGCAAGGAGGCCTACAAACCTGACTCAGTTACACCAGCTCTGT | |
| | CAGGAGGAATGGGCCAAAATTCACCCAACTTATTGTGGGAAGCTTGTG | |
| | GAAGGCTACCCGAAACGTTTGACCCAAGTTAAACAATTTAAAGGCAAT | |
| | GCTACCAAATAC | |

### 5.8 Immune Effector Cells and Methods of Engineering

In one aspect, provided herein are cells, e.g., immune effector cells, comprising a recombinant vector comprising a polycistronic expression cassette (e.g., a vector described herein). In some embodiments, the immune effector cell is a T cell. For example, in certain embodiments, the T cell is selected from the group consisting of a naïve T cell (CD4+ or CD8+); a killer CD8+ T cell; a cytotoxic CD4+ T cell; a CD4+ T cell corresponding to Th1, Th2, Th9, Th17, Th22, follicular helper (Tfh), regulatory (Treg) lineages; a CD8⁺ cytotoxic T cell, a CD4⁺ cytotoxic T cell; a CD4⁺ helper T cell (e.g., a Th1 or a Th2 cell); a CD4/CD8 double positive T cell; a tumor infiltrating T cell (TIL); a thymocyte; a memory T cell, (e.g., a central memory T cell, an effector memory T cell, a stem cell-like memory T cell, or a stem cell memory T cell), and a natural killer T cell, e.g., an invariant natural killer T cell. In some embodiments, the T cell is a CD39^{neg}CD69^{neg} T cell or a CD8⁺CD39^{neg}CD69^{neg} cell, as described, e.g., in Krishna et al., “Stem-like CD8 T cells mediate response of adoptive cell immunotherapy against human cancer,” 2020 370(6522):1328-1334, which is incorporated by reference herein in its entirety. Precursor cells of the cellular immune system (e.g., precursors of T lymphocytes) are also useful for presenting a TCR disclosed herein because these cells may differentiate, develop, or mature into effector cells. Accordingly, in certain embodiments, the mammalian cell is a pluripotent stem cell (e.g., an embryonic stem cell, an induced pluripotent stem cell), a hematopoietic stem cell, or a lymphocyte progenitor cell. In certain embodiments, the hematopoietic stem cell or lymphocyte progenitor cell is isolated and/or enriched from, e.g., bone marrow, umbilical cord blood, or peripheral blood. In some embodiments, the immune effector cell is a CD4+ T cell. In some embodiments, the immune effector cell is a CD8+ T cell. In one aspect, provided herein is a population of immune effector cells comprising a polycistronic vector described herein. In some embodiments, the population of immune effector cells comprises CD4+ T cells and CD8+ T cells. In some embodiments, the population of immune effector cells are an ex vivo culture.

In one aspect, provided herein are methods of introducing a vector described herein into a plurality of cells, e.g., immune effector cells, to produce a plurality of engineered cells, e.g., immune effector cells. Methods of introducing vectors into a cell are well known in the art. In the context of an expression vector, the vector can be readily introduced into a host cell, e.g., mammalian (e.g., human) cell by any method in the art. For example, the expression vector can be transferred into a host cell by transfection or transduction. Exemplary methods for introducing a vector into a host cell, include, but are not limited to, electroporation (also referred to herein as electro-transfer), sonication, calcium phosphate precipitation, lipofection, particle bombardment, microinjection, mechanical deformation by passage through a microfluidic device, and the like, see, e.g., Sambrook et al. Molecular Cloning: A Laboratory Manual, Cold Spring Harbor Laboratory, New York (2001), the entire contents of which is incorporated by reference herein. In some embodiments, a polycistronic vector is introduced into an immune effector cell or population of immune effector cells via electroporation. Alternative delivery systems include, e.g., colloidal dispersion systems, such as macromolecule complexes, nanocapsules, microspheres, beads, and lipid-based systems including oil-in-water emulsions, micelles, mixed micelles, and liposomes. In some embodiments, the polycistronic vector is introduced into a population of cells, e.g., immune effector cells, ex vivo, in vitro, or in vivo. In some embodiments, the polycistronic vector is introduced into a population of cells, e.g., immune effector cells, ex vivo.

In some embodiments, co-expression of mbIL-15 with a transgenic TCR in T cells produces a final drug product that contains T stem cell memory cells (Tscm) which are capable of self-renewal and differentiation into other effector T cell subsets. The expression of mbIL-15 on T cells maintains a population of self-renewing T stem cell memory or T stem cell memory like (Tscm-like) cells that are defined by the surface marker phenotype CD45RA+CD45RO-CD62L+CD95+ or CD45RA+CD45RO+CD62L+CD95+, respectively. In some embodiments, expression of mbIL-15 on T cells is able to maintain Tscm or Tscm-like subsets as defined above in the absence of external growth and survival factors (i.e., cytokines or antigen stimulation).

In some embodiments, populations of T cells co-expressing mbIL-15 with a transgenic TCR produced by the tricistronic vectors described herein comprise more than 5%, 10%, 15%, 20%, 25%, 30%, 35%, 40%, 45% or 50% Tscm cells. In some embodiments, populations of T cells co-expressing mbIL-15 with a transgenic TCR produced by the tricistronic vectors described herein comprise more than 5%, 10%, 15%, 20%, 25%, 30%, 35%, 40%, 45% or 50% Tscm-like cells. In some embodiments, populations of T cells co-expressing mbIL-15 with a transgenic TCR produced by the tricistronic vectors described herein comprise more than 5%, 10%, 15%, 20%, 25%, 30%, 35%, 40%, 45% or 50% CD45RA+CD45RO−CD62L+CD95+ cells. In some embodiments, populations of T cells co-expressing mbIL-15 with a transgenic TCR produced by the tricistronic vectors described herein comprise more than 5%, 10%, 15%, 20%, 25%, 30%, 35%, 40%, 45% or 50% CD45RA+CD45RO+CD62L+CD95+ cells.

### 5.8.1 Sources of Immune Effector Cells

Immune effector cells may be obtained from a subject by any suitable method known in the art. For example, T cells (e.g., CD4+ T cells and CD8+ T cells) can be obtained from several sources, including peripheral blood mononuclear cells, bone marrow, lymph node tissue, cord blood, thymus tissue, tissue from a site of infection, ascites, pleural effusion, spleen tissue, and tumors. In some embodiments, immune effector cells (e.g., T cells) are obtained from blood collected from a subject using any number of techniques known to the skilled artisan. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. T cells are isolated from peripheral blood lymphocytes by lysing the red blood cells and depleting the monocytes, for example, by centrifugation through a Percoll gradient or by counter flow centrifugal elutriation.

The cells collected by apheresis can be washed to remove the plasma fraction and to place the cells in an appropriate buffer (e.g., phosphate buffered saline (PBS)) or media for subsequent processing steps. The washing step may be accomplished by methods known to those in the art, such as by using a semi-automated “flow-through” centrifuge. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

A specific subpopulation of cells can be further isolated by positive or negative selection techniques (e.g., antibody coated beads, flow cytometry, etc.). In some embodiments, a specific subpopulation of T cells, such as CD3+, CD28+, CD4+, CD8+, CD45RA+, and CD45RO+ T cells, can be further isolated by positive or negative selection techniques (e.g., antibody coated beads, flow cytometry, etc.).

In certain embodiments, the mammalian cell is a population of cells presenting a TCR disclosed herein on the cell surface. The population of cells can be heterogeneous or homogenous. In certain embodiments, at least 50% (e.g., at least 60%, 70%, 80%, 90%, 95%, 99%, 99.5%, or 99.9%) of the population is a cell as described herein. In certain embodiments, the population is substantially pure, wherein at least 50% (e.g., at least 60%, 70%, 80%, 90%, 95%, 99%, 99.5%, or 99.9%) of the population is homogeneous. In certain embodiments, the population is heterogeneous and comprises a mixed population of cells (e.g., the cells have different cell types, developmental stages, origins, are isolated, purified, or enriched by different methods, are stimulated with different agents, and/or are engineered by different methods). In certain embodiments, the cells are a population of peripheral blood mononuclear cells (PBMC) (e.g., human PBMCs).

Populations of cells can be enriched or purified, as needed. In certain embodiments, regulatory T cells (e.g., CD25⁺ T cells) are depleted from the population, e.g., by using an anti-CD25 antibody conjugated to a surface such as a bead, particle, or cell. In certain embodiments, an anti-CD25 antibody is conjugated to a fluorescent dye (e.g., for use in fluorescence-activated cell sorting). In certain embodiments, cells expressing checkpoint receptors (e.g., CTLA-4, PD-1, TIM-3, LAG-3, TIGIT, VISTA, BTLA, TIGIT, CD137, or CEACAM1) are depleted from the population, e.g., by using an antibody that binds specifically to a checkpoint receptor conjugated to a surface such as a bead, particle, or cell. In certain embodiments, a T cell population can be selected so that it expresses one or more of IFN-γ, TNFα, IL-17A, IL-2, IL-3, IL-4, GM-CSF, IL-13, granzyme (e.g., granzyme B), and perforin, or other appropriate molecules, e.g., other cytokines. Methods for determining such expression are described, for example, in PCT Publication No.: WO 2013/126712, which is incorporated by reference herein in its entirety.

### 5.8.2 Methods of Manufacture

Engineered cells described herein can be manufactured by any method known in the art. Exemplary methods are shown in U.S. Patent Publication No. 2020/0347350 and International Publication No. WO 2019/067242, incorporated by reference in their entireties.

### 5.9 Pharmaceutical Compositions

Provided herein are pharmaceutical compositions comprising a population of engineered immune effector cells disclosed herein having the desired degree of purity in a physiologically acceptable carrier, excipient or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutical compositions described herein can be useful in inducing an immune response in a subject and treating a condition, such as cancer. In one embodiment, the present disclosure provides a pharmaceutical composition comprising a population of engineered immune effector cells described herein for use as a medicament. In another embodiment, the disclosure provides a pharmaceutical composition for use in a method for the treatment of cancer. In some embodiments, pharmaceutical compositions comprise a population of engineered immune effector cells disclosed herein, and optionally one or more additional prophylactic or therapeutic agents, in a pharmaceutically acceptable carrier.

A pharmaceutical composition may be formulated for any route of administration to a subject. Specific examples of routes of administration include parenteral administration (e.g., intravenous, subcutaneous, intramuscular). In some embodiments, the pharmaceutical composition is formulated for intravenous administration. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In some embodiments, the pharmaceutical composition is formulated for intravenous administration. Suitable carriers for intravenous administration include physiological saline or phosphate buffered saline (PBS), and solutions containing thickening and solubilizing agents, such as glucose, polyethylene glycol, and polypropylene glycol and mixtures thereof.

The compositions to be used for in vivo administration can be sterile. This is readily accomplished by filtration through, e.g., sterile filtration membranes.

Pharmaceutically acceptable carriers used in parenteral preparations include for example, aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, Ringer's injection, isotonic dextrose injection, sterile water injection, dextrose and lactated Ringer's injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether treatment is prophylactic or therapeutic. Treatment dosages are optimally titrated to optimize safety and efficacy.

### 5.10 Kits

In one aspect, provided herein are kits comprising one or more pharmaceutical composition, population of engineered effector cells (e.g., recombinant cells), polynucleotide, or vector described herein and instructions for use. Such kits may include, e.g., a carrier, package, or container that is compartmentalized to receive one or more containers such as vials, tubes, and the like. Suitable containers include, for example, bottles, vials, syringes, and test tubes. In one embodiment, the containers are formed from a variety of materials such as glass or plastic.

In a specific embodiment, provided herein is a pharmaceutical kit comprising one or more containers filled with one or more of the ingredients of the pharmaceutical compositions described herein, population of engineered immune effector cells, polynucleotides, or vectors provided herein. In one embodiment, the kit comprises a pharmaceutical composition comprising a population of engineered immune effector cells described herein. In one embodiment, the kit comprises a pharmaceutical composition comprising a population of immune effector cells engineered according to a method described herein. In some embodiments, the kit contains a pharmaceutical composition described herein and a prophylactic or therapeutic agent. Optionally associated with such container(s) can be a notice in the form prescribed by a governmental agency regulating the manufacture, use or sale of pharmaceuticals or biological products, which notice reflects approval by the agency of manufacture, use or sale for human administration. 6. EXAMPLES

The examples of the present disclosure are offered by way of illustration and explanation, and are not intended to limit the scope of the present disclosure.

### 6.1 Example 1: Construction of Transposon Plasmids

To improve homogeneity of multigene co-expression and product manufacturability, recombinant nucleic acid SB transposon plasmids comprising polycistronic expression cassettes were constructed. The polycistronic expression cassettes each include a transcriptional regulatory element operably linked to a polycistronic polynucleotide that encodes the TCR α chain of TCR001 (referred to herein as “TCRα” or “A”), the TCR β chain of TCR001 (referred to herein as “TCRβ” or “B”), and membrane-bound IL-15/IL-15Rα fusion protein (referred to herein as “mbIL15” or “15”), each separated by a furin recognition site and either a P2A element or a T2A element that mediates ribosome skipping to enable expression of separate polypeptide chains.

The TCR used in this Example, TCR001, is a chimeric TCR with murine-derived constant regions and with human Vα and Vβ regions specific for the R175H mutation of the p53 protein (in which position 175 of the p53 protein is mutated from Arg to His) in the context of HLA-A*02:01.

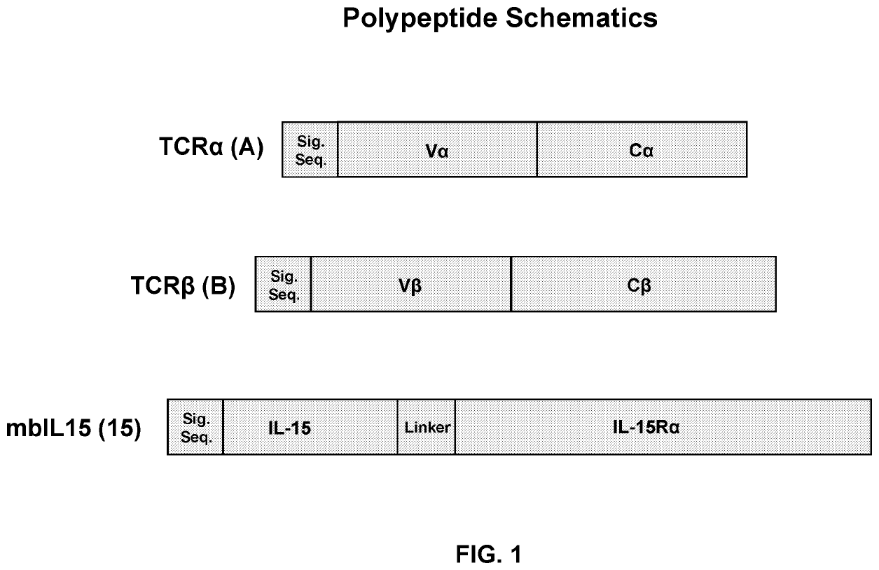

Briefly, TCRα was generated by fusing a human Vα region, including its N-terminal signal sequence (SEQ ID NO: 1006) with a glutamic acid at position 2, to a murine Cα region modified by substituting a cysteine at amino acid position 48, a leucine at amino acid position 112, an isoleucine at amino acid position 114, and a valine at amino acid position 115 (SEQ ID NO: 41). TCRβ was generated by fusing a human Vβ region, including its N-terminal signal sequence (SEQ ID NO: 2006) with an alanine at position 2, to a murine Cβ modified by substituting a cysteine at amino acid position 57 (SEQ ID NO: 51). mbIL15 was constructed by joining human IL-15 (SEQ ID NO: 76) to human IL-15Rα (SEQ ID NO: 78) via a Gly-Ser-rich linker peptide (SEQ ID NO: 81), with an IgE signal sequence (SEQ ID NO: 83) N-terminal to the human IL-15. Schematics of each of these three polypeptide constructs are shown in FIG. 1, from N terminus (left) to C terminus (right) for each construct.

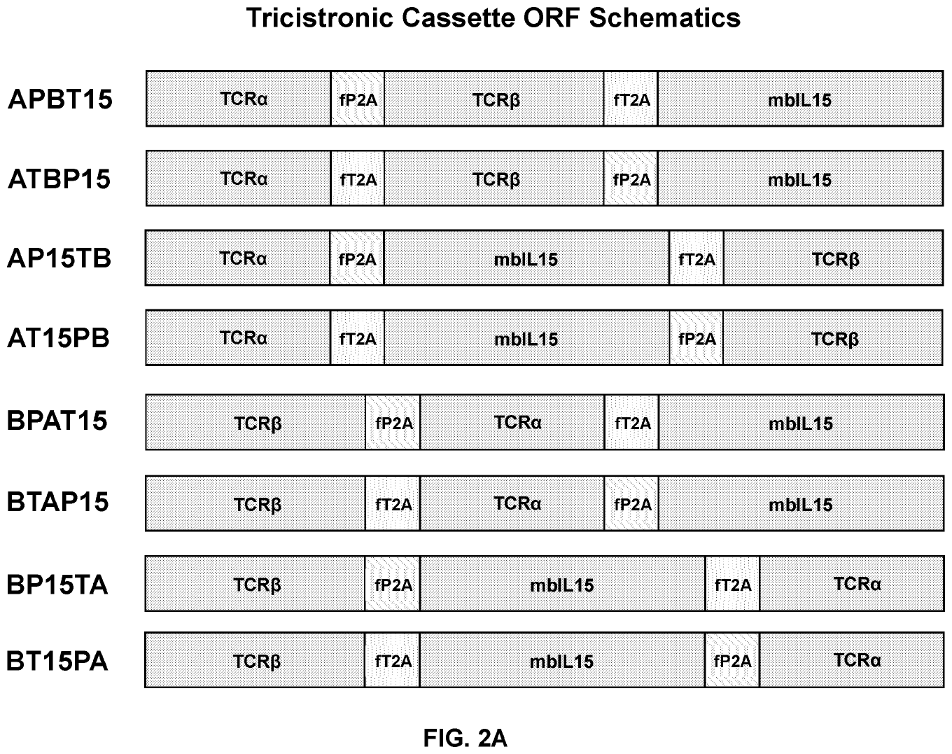

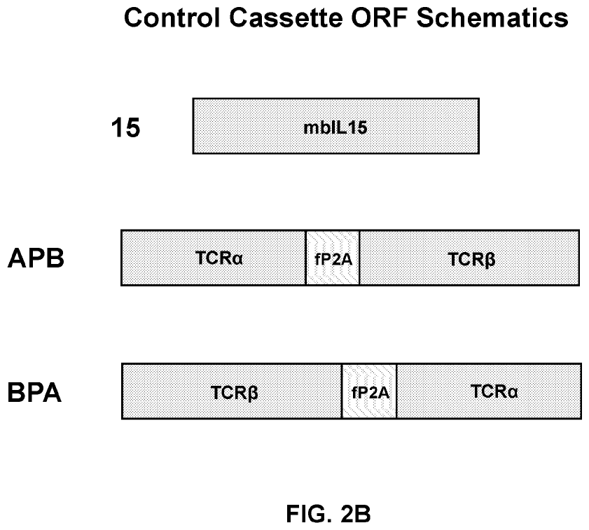

To explore the effect of gene/element order on expression and function, eight tricistronic polynucleotide expression cassettes were generated with polynucleotides encoding each of TCRα, TCRβ, and mbIL15. In each expression cassette, these three elements were fused pairwise with a) a polynucleotide encoding a furin recognition site joined to a P2A element (SEQ ID NO: 11) (referred to herein as “fP2A” or “P”) and b) a polynucleotide encoding a furin recognition site joined to a T2A element (SEQ ID NO: 13) (referred to herein as “fT2A” or “T”). The resulting tricistronic expression cassettes, including suitable transcriptional regulatory elements, were inserted between the ITRs of Sleeping Beauty (SB) transposon plasmids. The 5′ to 3′ order of elements in the open reading frame (ORF) of each expression cassette and SB Plasmid is shown in Table E1, and schematics of the ORFs of these eight expression cassettes are shown in FIG. 2A.

TABLE E1
Tricistronic SB transposon plasmids.

| Plasmid Name | Cassette Name | Order of Elements (5′ to 3′) |
|---|---|---|
| Plasmid APBT15 | Cassette APBT15 | TCRα-fP2A-TCRβ-fT2A-mbIL15 |
| Plasmid ATBP15 | Cassette ATBP15 | TCRα-fT2A-TCRβ-fP2A-mbIL15 |
| Plasmid AP15TB | Cassette AP15TB | TCRα-fP2A- mbIL15-fT2A-TCRβ |
| Plasmid AT15PB | Cassette AT15PB | TCRα-fT2A- mbIL15-fP2A-TCRβ |
| Plasmid BPAT15 | Cassette BPAT15 | TCRβ-fP2A-TCRα-fT2A-mbIL15 |
| Plasmid BTAP15 | Cassette BTAP15 | TCRβ-fT2A-TCRα-fP2A-mbIL15 |
| Plasmid BP15TA | Cassette BP15TA | TCRβ-fP2A- mbIL15-fT2A-TCRα |
| Plasmid BT15PA | Cassette BT15PA | TCRβ-fT2A- mbIL15-fP2A-TCRα |

The polynucleotide sequences of the ORFs of these transposon plasmids is shown in Table E2.

TABLE E2
Polynucleotide sequences of SB plasmid ORFs.

| | | SEQ |
|---|---|---|
| | | ID |
| Plasmid | Polynucleotide Sequence of ORF | NO: |
| Plasmid | ATGGAGTCCTTTCTGGGCGGCGTGCTGCTGATCCTGTGGCTGCAGGTGGACTGGGTGAAAT | 320 |
| APBT15 | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGCGACCAATTTCAGCCTGCTGAAGCA | |
| | GGCGGGCGATGTGGAGGAGAACCCTGGCCCAATGGCGACAAGACTGCTGTGCTGGGCGGCG | |
| | CTGTGCCTGCTGGGAGCGGAACTGACTGAAGCGGGGGTCGCGCAGAGCCCTCGATACAAAA | |
| | TCATTGAGAAGCGGCAGTCTGTGGCGTTCTGGTGCAACCCAATCAGCGGACACGCGACCCT | |
| | GTACTGGTATCAGCAGATCCTGGGCCAGGGCCCTAAGCTGCTGATTCAGTTCCAGAACAAT | |
| | GGCGTGGTGGACGATAGCCAGCTGCCAAAAGATAGATTTTCCGCGGAGAGGCTGAAGGGCG | |
| | TGGACTCTACACTGAAAATTCAGCCTGCGAAGCTGGAGGATAGCGCGGTGTACCTGTGCGC | |
| | GAGCTCCCTGGACCCAGGCGATACCGGAGAGCTGTTCTTTGGAGAGGGCAGCCGGCTGACA | |
| | GTGCTGGAGGACCTGAGGAACGTGACCCCACCTAAAGTGAGCCTGTTCGAGCCATCCAAGG | |
| | CGGAGATCGCGAATAAGCAGAAAGCGACCCTGGTGTGCCTGGCGAGGGGCTTCTTTCCCGA | |
| | TCACGTGGAGCTGTCCTGGTGGGTGAACGGCAAAGAGGTGCACTCTGGCGTGTGCACAGAC | |
| | CCTCAGGCGTACAAGGAGAGCAATTACTCCTATTGTCTGTCTAGCAGACTGAGGGTGAGCG | |
| | CGACCTTTTGGCACAACCCCCGGAATCACTTCCGCTGCCAGGTGCAGTTTCACGGCCTGTC | |
| | CGAGGAGGATAAATGGCCTGAGGGCTCTCCAAAGCCCGTGACACAGAATATCAGCGCGGAG | |
| | GCGTGGGGAAGAGCGGACTGTGGCATTACAAGCGCGTCCTATCAGCAGGGCGTGCTGTCCG | |
| | CGACCATCCTGTACGAGATTCTGCTGGGCAAGGCGACACTGTATGCGGTGCTGGTGTCCAC | |
| | CCTGGTGGTCATGGCGATGGTGAAGAGGAAAAACTCTCGGGCGAAACGCTCTGGAAGCGGA | |
| | GAGGGCAGAGGAAGTCTTCTAACATGCGGTGACGTGGAGGAGAATCCCGGCCCTATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGAGTCCTTTCTGGGCGGCGTGCTGCTGATCCTGTGGCTGCAGGTGGACTGGGTGAAAT | 321 |
| ATBP15 | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGAGGGCAGAGGAAGTCTTCTAACATG | |
| | CGGTGACGTGGAGGAGAATCCCGGCCCTATGGCGACAAGACTGCTGTGCTGGGCGGCGCTG | |
| | TGCCTGCTGGGAGCGGAACTGACTGAAGCGGGGGTCGCGCAGAGCCCTCGATACAAAATCA | |
| | TTGAGAAGCGGCAGTCTGTGGCGTTCTGGTGCAACCCAATCAGCGGACACGCGACCCTGTA | |
| | CTGGTATCAGCAGATCCTGGGCCAGGGCCCTAAGCTGCTGATTCAGTTCCAGAACAATGGC | |
| | GTGGTGGACGATAGCCAGCTGCCAAAAGATAGATTTTCCGCGGAGAGGCTGAAGGGCGTGG | |
| | ACTCTACACTGAAAATTCAGCCTGCGAAGCTGGAGGATAGCGCGGTGTACCTGTGCGCGAG | |
| | CTCCCTGGACCCAGGCGATACCGGAGAGCTGTTCTTTGGAGAGGGCAGCCGGCTGACAGTG | |
| | CTGGAGGACCTGAGGAACGTGACCCCACCTAAAGTGAGCCTGTTCGAGCCATCCAAGGCGG | |
| | AGATCGCGAATAAGCAGAAAGCGACCCTGGTGTGCCTGGCGAGGGGCTTCTTTCCCGATCA | |
| | CGTGGAGCTGTCCTGGTGGGTGAACGGCAAAGAGGTGCACTCTGGCGTGTGCACAGACCCT | |
| | CAGGCGTACAAGGAGAGCAATTACTCCTATTGTCTGTCTAGCAGACTGAGGGTGAGCGCGA | |
| | CCTTTTGGCACAACCCCCGGAATCACTTCCGCTGCCAGGTGCAGTTTCACGGCCTGTCCGA | |
| | GGAGGATAAATGGCCTGAGGGCTCTCCAAAGCCCGTGACACAGAATATCAGCGCGGAGGCG | |
| | TGGGGAAGAGCGGACTGTGGCATTACAAGCGCGTCCTATCAGCAGGGCGTGCTGTCCGCGA | |
| | CCATCCTGTACGAGATTCTGCTGGGCAAGGCGACACTGTATGCGGTGCTGGTGTCCACCCT | |
| | GGTGGTCATGGCGATGGTGAAGAGGAAAAACTCTCGGGCGAAACGCTCTGGAAGCGGAGCG | |
| | ACCAATTTCAGCCTGCTGAAGCAGGCGGGCGATGTGGAGGAGAACCCTGGCCCAATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGAGTCCTTTCTGGGCGGCGTGCTGCTGATCCTGTGGCTGCAGGTGGACTGGGTGAAAT | 322 |
| AP15TB | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGCGACCAATTTCAGCCTGCTGAAGCA | |
| | GGCGGGCGATGTGGAGGAGAACCCTGGCCCAATGGATTGGACCTGGATTCTGTTTCTGGTG | |
| | GCCGCTGCCACAAGAGTGCACAGCAACTGGGTGAATGTGATCAGCGACCTGAAGAAGATCG | |
| | AGGATCTGATCCAGAGCATGCACATTGATGCCACCCTGTACACAGAATCTGATGTGCACCC | |
| | TAGCTGTAAAGTGACCGCCATGAAGTGTTTTCTGCTGGAGCTGCAGGTGATTTCTCTGGAA | |
| | AGCGGAGATGCCTCTATCCACGACACAGTGGAGAATCTGATCATCCTGGCCAACAATAGCC | |
| | TGAGCAGCAATGGCAATGTGACAGAGTCTGGCTGTAAGGAGTGTGAGGAGCTGGAGGAGAA | |
| | GAACATCAAGGAGTTTCTGCAGAGCTTTGTGCACATCGTGCAGATGTTCATCAATACAAGC | |
| | TCTGGCGGAGGATCTGGAGGAGGCGGATCTGGAGGAGGAGGCAGTGGAGGCGGAGGATCTG | |
| | GCGGAGGATCTCTGCAGATTACATGCCCTCCTCCAATGTCTGTGGAGCACGCCGATATTTG | |
| | GGTGAAGTCCTACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAATGTGGCCCACT | |
| | GGACAACACCTAGCCTGAAGTGCATTAGAGATCCTGCCCTGGTCCACCAGAGGCCTGCCCC | |
| | TCCATCTACAGTGACAACAGCCGGAGTGACACCTCAGCCTGAATCTCTGAGCCCTTCTGGA | |
| | AAAGAACCTGCCGCCAGCTCTCCTAGCTCTAATAATACCGCCGCCACAACAGCCGCCATTG | |
| | TGCCTGGATCTCAGCTGATGCCTAGCAAGTCTCCTAGCACAGGCACAACAGAGATCAGCAG | |
| | CCACGAATCTTCTCACGGAACACCTTCTCAGACCACCGCCAAGAATTGGGAGCTGACAGCC | |
| | TCTGCCTCTCACCAGCCTCCAGGAGTGTATCCTCAGGGCCACTCTGATACAACAGTGGCCA | |
| | TCAGCACATCTACAGTGCTGCTGTGTGGACTGTCTGCCGTGTCTCTGCTGGCCTGTTACCT | |
| | GAAGTCTAGACAGACACCTCCTCTGGCCTCTGTGGAGATGGAGGCCATGGAAGCCCTGCCT | |
| | GTGACATGGGGAACAAGCAGCAGAGATGAAGACCTGGAGAATTGTTCTCACCACCTGCGGG | |
| | CGAAACGCTCTGGAAGCGGAGAGGGCAGAGGAAGTCTTCTAACATGCGGTGACGTGGAGGA | |
| | GAATCCCGGCCCTATGGCGACAAGACTGCTGTGCTGGGCGGCGCTGTGCCTGCTGGGAGCG | |
| | GAACTGACTGAAGCGGGGGTCGCGCAGAGCCCTCGATACAAAATCATTGAGAAGCGGCAGT | |
| | CTGTGGCGTTCTGGTGCAACCCAATCAGCGGACACGCGACCCTGTACTGGTATCAGCAGAT | |
| | CCTGGGCCAGGGCCCTAAGCTGCTGATTCAGTTCCAGAACAATGGCGTGGTGGACGATAGC | |
| | CAGCTGCCAAAAGATAGATTTTCCGCGGAGAGGCTGAAGGGCGTGGACTCTACACTGAAAA | |
| | TTCAGCCTGCGAAGCTGGAGGATAGCGCGGTGTACCTGTGCGCGAGCTCCCTGGACCCAGG | |
| | CGATACCGGAGAGCTGTTCTTTGGAGAGGGCAGCCGGCTGACAGTGCTGGAGGACCTGAGG | |
| | AACGTGACCCCACCTAAAGTGAGCCTGTTCGAGCCATCCAAGGCGGAGATCGCGAATAAGC | |
| | AGAAAGCGACCCTGGTGTGCCTGGCGAGGGGCTTCTTTCCCGATCACGTGGAGCTGTCCTG | |
| | GTGGGTGAACGGCAAAGAGGTGCACTCTGGCGTGTGCACAGACCCTCAGGCGTACAAGGAG | |
| | AGCAATTACTCCTATTGTCTGTCTAGCAGACTGAGGGTGAGCGCGACCTTTTGGCACAACC | |
| | CCCGGAATCACTTCCGCTGCCAGGTGCAGTTTCACGGCCTGTCCGAGGAGGATAAATGGCC | |
| | TGAGGGCTCTCCAAAGCCCGTGACACAGAATATCAGCGCGGAGGCGTGGGGAAGAGCGGAC | |
| | TGTGGCATTACAAGCGCGTCCTATCAGCAGGGCGTGCTGTCCGCGACCATCCTGTACGAGA | |
| | TTCTGCTGGGCAAGGCGACACTGTATGCGGTGCTGGTGTCCACCCTGGTGGTCATGGCGAT | |
| | GGTGAAGAGGAAAAACTCT | |
| Plasmid | ATGGAGTCCTTTCTGGGCGGCGTGCTGCTGATCCTGTGGCTGCAGGTGGACTGGGTGAAAT | 323 |
| AT15PB | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGAGGGCAGAGGAAGTCTTCTAACATG | |
| | CGGTGACGTGGAGGAGAATCCCGGCCCTATGGATTGGACCTGGATTCTGTTTCTGGTGGCC | |
| | GCTGCCACAAGAGTGCACAGCAACTGGGTGAATGTGATCAGCGACCTGAAGAAGATCGAGG | |
| | ATCTGATCCAGAGCATGCACATTGATGCCACCCTGTACACAGAATCTGATGTGCACCCTAG | |
| | CTGTAAAGTGACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAACAATAGCCTGA | |
| | GCAGCAATGGCAATGTGACAGAGTCTGGCTGTAAGGAGTGTGAGGAGCTGGAGGAGAAGAA | |
| | CATCAAGGAGTTTCTGCAGAGCTTTGTGCACATCGTGCAGATGTTCATCAATACAAGCTCT | |
| | GGCGGAGGATCTGGAGGAGGCGGATCTGGAGGAGGAGGCAGTGGAGGCGGAGGATCTGGCG | |
| | GAGGATCTCTGCAGATTACATGCCCTCCTCCAATGTCTGTGGAGCACGCCGATATTTGGGT | |
| | GAAGTCCTACAGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTGGCCCACTGGA | |
| | CAACACCTAGCCTGAAGTGCATTAGAGATCCTGCCCTGGTCCACCAGAGGCCTGCCCCTCC | |
| | ATCTACAGTGACAACAGCCGGAGTGACACCTCAGCCTGAATCTCTGAGCCCTTCTGGAAAA | |
| | GAACCTGCCGCCAGCTCTCCTAGCTCTAATAATACCGCCGCCACAACAGCCGCCATTGTGC | |
| | CTGGATCTCAGCTGATGCCTAGCAAGTCTCCTAGCACAGGCACAACAGAGATCAGCAGCCA | |
| | CGAATCTTCTCACGGAACACCTTCTCAGACCACCGCCAAGAATTGGGAGCTGACAGCCTCT | |
| | GCCTCTCACCAGCCTCCAGGAGTGTATCCTCAGGGCCACTCTGATACAACAGTGGCCATCA | |
| | GCACATCTACAGTGCTGCTGTGTGGACTGTCTGCCGTGTCTCTGCTGGCCTGTTACCTGAA | |
| | GTCTAGACAGACACCTCCTCTGGCCTCTGTGGAGATGGAGGCCATGGAAGCCCTGCCTGTG | |
| | ACATGGGGAACAAGCAGCAGAGATGAAGACCTGGAGAATTGTTCTCACCACCTGCGGGCGA | |
| | AACGCTCTGGAAGCGGAGCGACCAATTTCAGCCTGCTGAAGCAGGCGGGCGATGTGGAGGA | |
| | GAACCCTGGCCCAATGGCGACAAGACTGCTGTGCTGGGCGGCGCTGTGCCTGCTGGGAGCG | |
| | GAACTGACTGAAGCGGGGGTCGCGCAGAGCCCTCGATACAAAATCATTGAGAAGCGGCAGT | |
| | CTGTGGCGTTCTGGTGCAACCCAATCAGCGGACACGCGACCCTGTACTGGTATCAGCAGAT | |
| | CCTGGGCCAGGGCCCTAAGCTGCTGATTCAGTTCCAGAACAATGGCGTGGTGGACGATAGC | |
| | CAGCTGCCAAAAGATAGATTTTCCGCGGAGAGGCTGAAGGGCGTGGACTCTACACTGAAAA | |
| | TTCAGCCTGCGAAGCTGGAGGATAGCGCGGTGTACCTGTGCGCGAGCTCCCTGGACCCAGG | |
| | CGATACCGGAGAGCTGTTCTTTGGAGAGGGCAGCCGGCTGACAGTGCTGGAGGACCTGAGG | |
| | AACGTGACCCCACCTAAAGTGAGCCTGTTCGAGCCATCCAAGGCGGAGATCGCGAATAAGC | |
| | AGAAAGCGACCCTGGTGTGCCTGGCGAGGGGCTTCTTTCCCGATCACGTGGAGCTGTCCTG | |
| | GTGGGTGAACGGCAAAGAGGTGCACTCTGGCGTGTGCACAGACCCTCAGGCGTACAAGGAG | |
| | AGCAATTACTCCTATTGTCTGTCTAGCAGACTGAGGGTGAGCGCGACCTTTTGGCACAACC | |
| | CCCGGAATCACTTCCGCTGCCAGGTGCAGTTTCACGGCCTGTCCGAGGAGGATAAATGGCC | |
| | TGAGGGCTCTCCAAAGCCCGTGACACAGAATATCAGCGCGGAGGCGTGGGGAAGAGCGGAC | |
| | TGTGGCATTACAAGCGCGTCCTATCAGCAGGGCGTGCTGTCCGCGACCATCCTGTACGAGA | |
| | TTCTGCTGGGCAAGGCGACACTGTATGCGGTGCTGGTGTCCACCCTGGTGGTCATGGCGAT | |
| | GGTGAAGAGGAAAAACTCT | |
| Plasmid | ATGGCGACAAGACTGCTGTGCTGGGCGGCGCTGTGCCTGCTGGGAGCGGAACTGACTGAAG | 324 |
| BPAT15 | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGCGACCAATTTCAGCCTGCTGAAGCAGGCGG | |
| | GCGATGTGGAGGAGAACCCTGGCCCAATGGAGTCCTTTCTGGGCGGCGTGCTGCTGATCCT | |
| | GTGGCTGCAGGTGGACTGGGTGAAATCCCAGAAGATCGAGCAGAACTCTGAGGCGCTGAAT | |
| | ATTCAGGAGGGCAAGACCGCGACACTGACCTGCAACTACACAAATTATTCCCCAGCGTACC | |
| | TGCAGTGGTATAGGCAGGACCCAGGCAGGGGACCCGTGTTTCTGCTGCTGATTCGGGAGAA | |
| | TGAGAAGGAGAAAAGAAAGGAGAGGCTGAAAGTGACCTTCGATACCACACTGAAGCAGTCT | |
| | CTGTTTCACATCACAGCGTCTCAGCCAGCGGACAGCGCGACCTACCTGTGCGCGCTGGACA | |
| | TCTACCCTCACGATATGAGATTCGGCGCGGGCACAAGGCTGACCGTGAAACCAAACATCCA | |
| | GAATCCCGAGCCTGCGGTGTACCAGCTGAAGGACCCCCGCTCTCAGGATAGCACACTGTGC | |
| | CTGTTCACCGACTTTGATAGCCAGATCAACGTGCCTAAAACAATGGAGTCCGGCACCTTCA | |
| | TCACCGACAAGTGCGTGCTGGATATGAAAGCGATGGACTCCAAGTCTAACGGCGCGATCGC | |
| | GTGGTCCAATCAGACATCTTTCACCTGCCAGGATATCTTCAAGGAGACAAACGCGACCTAT | |
| | CCTTCCTCTGACGTGCCATGTGATGCGACACTGACCGAGAAGAGCTTCGAGACAGACATGA | |
| | ACCTGAATTTTCAGAATCTGCTGGTCATCGTGCTGAGAATCCTGCTGCTGAAGGTGGCGGG | |
| | CTTTAATCTGCTGATGACACTGCGGCTGTGGAGTTCCCGGGCGAAACGCTCTGGAAGCGGA | |
| | GAGGGCAGAGGAAGTCTTCTAACATGCGGTGACGTGGAGGAGAATCCCGGCCCTATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGCGACAAGACTGCTGTGCTGGGCGGCGCTGTGCCTGCTGGGAGCGGAACTGACTGAAG | 325 |
| BTAP15 | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGAGGGCAGAGGAAGTCTTCTAACATGCGGTG | |
| | ACGTGGAGGAGAATCCCGGCCCTATGGAGTCCTTTCTGGGCGGCGTGCTGCTGATCCTGTG | |
| | GCTGCAGGTGGACTGGGTGAAATCCCAGAAGATCGAGCAGAACTCTGAGGCGCTGAATATT | |
| | CAGGAGGGCAAGACCGCGACACTGACCTGCAACTACACAAATTATTCCCCAGCGTACCTGC | |
| | AGTGGTATAGGCAGGACCCAGGCAGGGGACCCGTGTTTCTGCTGCTGATTCGGGAGAATGA | |
| | GAAGGAGAAAAGAAAGGAGAGGCTGAAAGTGACCTTCGATACCACACTGAAGCAGTCTCTG | |
| | TTTCACATCACAGCGTCTCAGCCAGCGGACAGCGCGACCTACCTGTGCGCGCTGGACATCT | |
| | ACCCTCACGATATGAGATTCGGCGCGGGCACAAGGCTGACCGTGAAACCAAACATCCAGAA | |
| | TCCCGAGCCTGCGGTGTACCAGCTGAAGGACCCCCGCTCTCAGGATAGCACACTGTGCCTG | |
| | TTCACCGACTTTGATAGCCAGATCAACGTGCCTAAAACAATGGAGTCCGGCACCTTCATCA | |
| | CCGACAAGTGCGTGCTGGATATGAAAGCGATGGACTCCAAGTCTAACGGCGCGATCGCGTG | |
| | GTCCAATCAGACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACAGACATGAACC | |
| | TGAATTTTCAGAATCTGCTGGTCATCGTGCTGAGAATCCTGCTGCTGAAGGTGGCGGGCTT | |
| | TAATCTGCTGATGACACTGCGGCTGTGGAGTTCCCGGGCGAAACGCTCTGGAAGCGGAGCG | |
| | ACCAATTTCAGCCTGCTGAAGCAGGCGGGCGATGTGGAGGAGAACCCTGGCCCAATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGCGACAAGACTGCTGTGCTGGGCGGCGCTGTGCCTGCTGGGAGCGGAACTGACTGAAG | 326 |
| BP15TA | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGCGACCAATTTCAGCCTGCTGAAGCAGGCGG | |
| | GCGATGTGGAGGAGAACCCTGGCCCAATGGATTGGACCTGGATTCTGTTTCTGGTGGCCGC | |
| | TGCCACAAGAGTGCACAGCAACTGGGTGAATGTGATCAGCGACCTGAAGAAGATCGAGGAT | |
| | CTGATCCAGAGCATGCACATTGATGCCACCCTGTACACAGAATCTGATGTGCACCCTAGCT | |
| | GTAAAGTGACCGCCATGAAGTGTTTTCTGCTGGAGCTGCAGGTGATTTCTCTGGAAAGCGG | |
| | AGATGCCTCTATCCACGACACAGTGGAGAATCTGATCATCCTGGCCAACAATAGCCTGAGC | |
| | AGCAATGGCAATGTGACAGAGTCTGGCTGTAAGGAGTGTGAGGAGCTGGAGGAGAAGAACA | |
| | TCAAGGAGTTTCTGCAGAGCTTTGTGCACATCGTGCAGATGTTCATCAATACAAGCTCTGG | |
| | CGGAGGATCTGGAGGAGGCGGATCTGGAGGAGGAGGCAGTGGAGGCGGAGGATCTGGCGGA | |
| | GGATCTCTGCAGATTACATGCCCTCCTCCAATGTCTGTGGAGCACGCCGATATTTGGGTGA | |
| | AGTCCTACAGCCTGTACAGCAGAGAGAGATACATCTGCAACAGCGGCTTTAAGAGAAAGGC | |
| | CGGCACCTCTTCTCTGACAGAGTGCGTGCTGAATAAGGCCACAAATGTGGCCCACTGGACA | |
| | ACACCTAGCCTGAAGTGCATTAGAGATCCTGCCCTGGTCCACCAGAGGCCTGCCCCTCCAT | |
| | CTACAGTGACAACAGCCGGAGTGACACCTCAGCCTGAATCTCTGAGCCCTTCTGGAAAAGA | |
| | ACCTGCCGCCAGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATCAGCAGCCACG | |
| | AATCTTCTCACGGAACACCTTCTCAGACCACCGCCAAGAATTGGGAGCTGACAGCCTCTGC | |
| | CTCTCACCAGCCTCCAGGAGTGTATCCTCAGGGCCACTCTGATACAACAGTGGCCATCAGC | |
| | ACATCTACAGTGCTGCTGTGTGGACTGTCTGCCGTGTCTCTGCTGGCCTGTTACCTGAAGT | |
| | CTAGACAGACACCTCCTCTGGCCTCTGTGGAGATGGAGGCCATGGAAGCCCTGCCTGTGAC | |
| | ATGGGGAACAAGCAGCAGAGATGAAGACCTGGAGAATTGTTCTCACCACCTGCGGGCGAAA | |
| | CGCTCTGGAAGCGGAGAGGGCAGAGGAAGTCTTCTAACATGCGGTGACGTGGAGGAGAATC | |
| | CCGGCCCTATGGAGTCCTTTCTGGGCGGCGTGCTGCTGATCCTGTGGCTGCAGGTGGACTG | |
| | GGTGAAATCCCAGAAGATCGAGCAGAACTCTGAGGCGCTGAATATTCAGGAGGGCAAGACC | |
| | GCGACACTGACCTGCAACTACACAAATTATTCCCCAGCGTACCTGCAGTGGTATAGGCAGG | |
| | ACCCAGGCAGGGGACCCGTGTTTCTGCTGCTGATTCGGGAGAATGAGAAGGAGAAAAGAAA | |
| | GGAGAGGCTGAAAGTGACCTTCGATACCACACTGAAGCAGTCTCTGTTTCACATCACAGCG | |
| | TCTCAGCCAGCGGACAGCGCGACCTACCTGTGCGCGCTGGACATCTACCCTCACGATATGA | |
| | GATTCGGCGCGGGCACAAGGCTGACCGTGAAACCAAACATCCAGAATCCCGAGCCTGCGGT | |
| | GTACCAGCTGAAGGACCCCCGCTCTCAGGATAGCACACTGTGCCTGTTCACCGACTTTGAT | |
| | AGCCAGATCAACGTGCCTAAAACAATGGAGTCCGGCACCTTCATCACCGACAAGTGCGTGC | |
| | TGGATATGAAAGCGATGGACTCCAAGTCTAACGGCGCGATCGCGTGGTCCAATCAGACATC | |
| | TTTCACCTGCCAGGATATCTTCAAGGAGACAAACGCGACCTATCCTTCCTCTGACGTGCCA | |
| | TGTGATGCGACACTGACCGAGAAGAGCTTCGAGACAGACATGAACCTGAATTTTCAGAATC | |
| | TGCTGGTCATCGTGCTGAGAATCCTGCTGCTGAAGGTGGCGGGCTTTAATCTGCTGATGAC | |
| | ACTGCGGCTGTGGAGTTCC | |
| Plasmid | ATGGCGACAAGACTGCTGTGCTGGGCGGCGCTGTGCCTGCTGGGAGCGGAACTGACTGAAG | 327 |
| BT15PA | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGAGGGCAGAGGAAGTCTTCTAACATGCGGTG | |
| | ACGTGGAGGAGAATCCCGGCCCTATGGATTGGACCTGGATTCTGTTTCTGGTGGCCGCTGC | |
| | CACAAGAGTGCACAGCAACTGGGTGAATGTGATCAGCGACCTGAAGAAGATCGAGGATCTG | |
| | ATCCAGAGCATGCACATTGATGCCACCCTGTACACAGAATCTGATGTGCACCCTAGCTGTA | |
| | AAGTGACCGCCATGAAGTGTTTTCTGCTGGAGCTGCAGGTGATTTCTCTGGAAAGCGGAGA | |
| | TGCCTCTATCCACGACACAGTGGAGAATCTGATCATCCTGGCCAACAATAGCCTGAGCAGC | |
| | AATGGCAATGTGACAGAGTCTGGCTGTAAGGAGTGTGAGGAGCTGGAGGAGAAGAACATCA | |
| | AGGAGTTTCTGCAGAGCTTTGTGCACATCGTGCAGATGTTCATCAATACAAGCTCTGGCGG | |
| | AGGATCTGGAGGAGGCGGATCTGGAGGAGGAGGCAGTGGAGGCGGAGGATCTGGCGGAGGA | |
| | TCTCTGCAGATTACATGCCCTCCTCCAATGTCTGTGGAGCACGCCGATATTTGGGTGAAGT | |
| | CCTACAGCCTGTACAGCAGAGAGAGATACATCTGCAACAGCGGCTTTAAGAGAAAGGCCGG | |
| | CACCTCTTCTCTGACAGAGTGCGTGCTGAATAAGGCCACAAATGTGGCCCACTGGACAACA | |
| | CCTAGCCTGAAGTGCATTAGAGATCCTGCCCTGGTCCACCAGAGGCCTGCCCCTCCATCTA | |
| | CAGTGACAACAGCCGGAGTGACACCTCAGCCTGAATCTCTGAGCCCTTCTGGAAAAGAACC | |
| | TGCCGCCAGCTCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGCAGCCACGAAT | |
| | CTTCTCACGGAACACCTTCTCAGACCACCGCCAAGAATTGGGAGCTGACAGCCTCTGCCTC | |
| | TCACCAGCCTCCAGGAGTGTATCCTCAGGGCCACTCTGATACAACAGTGGCCATCAGCACA | |
| | TCTACAGTGCTGCTGTGTGGACTGTCTGCCGTGTCTCTGCTGGCCTGTTACCTGAAGTCTA | |
| | GACAGACACCTCCTCTGGCCTCTGTGGAGATGGAGGCCATGGAAGCCCTGCCTGTGACATG | |
| | GGGAACAAGCAGCAGAGATGAAGACCTGGAGAATTGTTCTCACCACCTGCGGGCGAAACGC | |
| | TCTGGAAGCGGAGCGACCAATTTCAGCCTGCTGAAGCAGGCGGGCGATGTGGAGGAGAACC | |
| | CTGGCCCAATGGAGTCCTTTCTGGGCGGCGTGCTGCTGATCCTGTGGCTGCAGGTGGACTG | |
| | GGTGAAATCCCAGAAGATCGAGCAGAACTCTGAGGCGCTGAATATTCAGGAGGGCAAGACC | |
| | GCGACACTGACCTGCAACTACACAAATTATTCCCCAGCGTACCTGCAGTGGTATAGGCAGG | |
| | ACCCAGGCAGGGGACCCGTGTTTCTGCTGCTGATTCGGGAGAATGAGAAGGAGAAAAGAAA | |
| | GGAGAGGCTGAAAGTGACCTTCGATACCACACTGAAGCAGTCTCTGTTTCACATCACAGCG | |
| | TCTCAGCCAGCGGACAGCGCGACCTACCTGTGCGCGCTGGACATCTACCCTCACGATATGA | |
| | GATTCGGCGCGGGCACAAGGCTGACCGTGAAACCAAACATCCAGAATCCCGAGCCTGCGGT | |
| | GTACCAGCTGAAGGACCCCCGCTCTCAGGATAGCACACTGTGCCTGTTCACCGACTTTGAT | |
| | AGCCAGATCAACGTGCCTAAAACAATGGAGTCCGGCACCTTCATCACCGACAAGTGCGTGC | |
| | TGGATATGAAAGCGATGGACTCCAAGTCTAACGGCGCGATCGCGTGGTCCAATCAGACATC | |
| | TTTCACCTGCCAGGATATCTTCAAGGAGACAAACGCGACCTATCCTTCCTCTGACGTGCCA | |
| | TGTGATGCGACACTGACCGAGAAGAGCTTCGAGACAGACATGAACCTGAATTTTCAGAATC | |
| | TGCTGGTCATCGTGCTGAGAATCCTGCTGCTGAAGGTGGCGGGCTTTAATCTGCTGATGAC | |
| | ACTGCGGCTGTGGAGTTCC | |

The corresponding theoretical polypeptide translation product resulting from each ORF, not accounting for N-terminal signal sequence cleavage or ribosomal skipping at each P2A and T2A site, is shown in Table E3.

TABLE E3
Polypeptide sequences encoded by SB plasmid ORFs.

| | | SEQ |
|---|---|---|
| | | ID |
| Plasmid | Amino Acid Translation of ORF | NO: |
| Plasmid | MESFLGGVLLILWLQVDWVKSQKIEQNSEALNIQEGKTATLTCNYTNYSPAYLQWYRQDPG | 330 |
| APBT15 | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGFNLLMTLRLWSSRAKRSGSGATNFSLLKQAGDVEENPGPMATRLLCWAA | |
| | LCLLGAELTEAGVAQSPRYKIIEKRQSVAFWCNPISGHATLYWYQQILGQGPKLLIQFQNN | |
| | GVVDDSQLPKDRFSAERLKGVDSTLKIQPAKLEDSAVYLCASSLDPGDTGELFFGEGSRLT | |
| | VLEDLRNVTPPKVSLFEPSKAEIANKQKATLVCLARGFFPDHVELSWWVNGKEVHSGVCTD | |
| | PQAYKESNYSYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKNSRAKRSGSG | |
| | EGRGSLLTCGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MESFLGGVLLILWLQVDWVKSQKIEQNSEALNIQEGKTATLTCNYTNYSPAYLQWYRQDPG | 331 |
| ATBP15 | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGENLLMTLRLWSSRAKRSGSGEGRGSLLTCGDVEENPGPMATRLLCWAAL | |
| | CLLGAELTEAGVAQSPRYKIIEKRQSVAFWCNPISGHATLYWYQQILGQGPKLLIQFQNNG | |
| | VVDDSQLPKDRFSAERLKGVDSTLKIQPAKLEDSAVYLCASSLDPGDTGELFFGEGSRLTV | |
| | LEDLRNVTPPKVSLFEPSKAEIANKQKATLVCLARGFFPDHVELSWWVNGKEVHSGVCTDP | |
| | QAYKESNYSYCLSSRLRVSATFWHNPRNHFRCQVQFHGLSEEDKWPEGSPKPVTQNISAEA | |
| | WGRADCGITSASYQQGVLSATILYEILLGKATLYAVLVSTLVVMAMVKRKNSRAKRSGSGA | |
| | TNFSLLKQAGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MESFLGGVLLILWLQVDWVKSQKIEQNSEALNIQEGKTATLTCNYTNYSPAYLQWYRQDPG | 332 |
| AP15TB | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGFNLLMTLRLWSSRAKRSGSGATNFSLLKQAGDVEENPGPMDWTWILFLV | |
| | AAATRVHSNWVNVISDLKKIEDLIQSMHIDATLYTESDVHPSCKVTAMKCFLLELQVISLE | |
| | SGDASIHDTVENLIILANNSLSSNGNVTESGCKECEELEEKNIKEFLQSFVHIVQMFINTS | |
| | SGGGSGGGGSGGGGSGGGGSGGGSLQITCPPPMSVEHADIWVKSYSLYSRERYICNSGFKR | |
| | KAGTSSLTECVLNKATNVAHWTTPSLKCIRDPALVHQRPAPPSTVTTAGVTPQPESLSPSG | |
| | KEPAASSPSSNNTAATTAAIVPGSQLMPSKSPSTGTTEISSHESSHGTPSQTTAKNWELTA | |
| | SASHQPPGVYPQGHSDTTVAISTSTVLLCGLSAVSLLACYLKSRQTPPLASVEMEAMEALP | |
| | VTWGTSSRDEDLENCSHHLRAKRSGSGEGRGSLLTCGDVEENPGPMATRLLCWAALCLLGA | |
| | ELTEAGVAQSPRYKIIEKRQSVAFWCNPISGHATLYWYQQILGQGPKLLIQFQNNGVVDDS | |
| | QLPKDRFSAERLKGVDSTLKIQPAKLEDSAVYLCASSLDPGDTGELFFGEGSRLTVLEDLR | |
| | NVTPPKVSLFEPSKAEIANKQKATLVCLARGFFPDHVELSWWVNGKEVHSGVCTDPQAYKE | |
| | SNYSYCLSSRLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| Plasmid | MESFLGGVLLILWLQVDWVKSQKIEQNSEALNIQEGKTATLTCNYTNYSPAYLQWYRQDPG | 333 |
| AT15PB | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGFNLLMTLRLWSSRAKRSGSGEGRGSLLTCGDVEENPGPMDWTWILFLVA | |
| | AATRVHSNWVNVISDLKKIEDLIQSMHIDATLYTESDVHPSCKVTAMKCELLELQVISLES | |
| | GDASIHDTVENLIILANNSLSSNGNVTESGCKECEELEEKNIKEFLQSFVHIVQMFINTSS | |
| | GGGSGGGGSGGGGSGGGGSGGGSLQITCPPPMSVEHADIWVKSYSLYSRERYICNSGFKRK | |
| | AGTSSLTECVLNKATNVAHWTTPSLKCIRDPALVHQRPAPPSTVTTAGVTPQPESLSPSGK | |
| | EPAASSPSSNNTAATTAAIVPGSQLMPSKSPSTGTTEISSHESSHGTPSQTTAKNWELTAS | |
| | ASHQPPGVYPQGHSDTTVAISTSTVLLCGLSAVSLLACYLKSRQTPPLASVEMEAMEALPV | |
| | TWGTSSRDEDLENCSHHLRAKRSGSGATNFSLLKQAGDVEENPGPMATRLLCWAALCLLGA | |
| | ELTEAGVAQSPRYKIIEKRQSVAFWCNPISGHATLYWYQQILGQGPKLLIQFQNNGVVDDS | |
| | QLPKDRFSAERLKGVDSTLKIQPAKLEDSAVYLCASSLDPGDTGELFFGEGSRLTVLEDLR | |
| | NVTPPKVSLFEPSKAEIANKQKATLVCLARGFFPDHVELSWWVNGKEVHSGVCTDPQAYKE | |
| | SNYSYCLSSRLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| Plasmid | MATRLLCWAALCLLGAELTEAGVAQSPRYKIIEKRQSVAFWCNPISGHATLYWYQQILGQG | 334 |
| BPAT15 | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGATNFSLLKQAGDVEENPGPMESFLGGVLLILWLQVDWVKSQKIEQNSEALN | |
| | IQEGKTATLTCNYTNYSPAYLQWYRQDPGRGPVFLLLIRENEKEKRKERLKVTFDTTLKQS | |
| | LFHITASQPADSATYLCALDIYPHDMRFGAGTRLTVKPNIQNPEPAVYQLKDPRSQDSTLC | |
| | LFTDFDSQINVPKTMESGTFITDKCVLDMKAMDSKSNGAIAWSNQTSFTCQDIFKETNATY | |
| | PSSDVPCDATLTEKSFETDMNLNFQNLLVIVLRILLLKVAGENLLMTLRLWSSRAKRSGSG | |
| | EGRGSLLTCGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MATRLLCWAALCLLGAELTEAGVAQSPRYKIIEKRQSVAFWCNPISGHATLYWYQQILGQG | 335 |
| BTAP15 | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGEGRGSLLTCGDVEENPGPMESFLGGVLLILWLQVDWVKSQKIEQNSEALNI | |
| | QEGKTATLTCNYTNYSPAYLQWYRQDPGRGPVFLLLIRENEKEKRKERLKVTFDTTLKQSL | |
| | FHITASQPADSATYLCALDIYPHDMRFGAGTRLTVKPNIQNPEPAVYQLKDPRSQDSTLCL | |
| | FTDFDSQINVPKTMESGTFITDKCVLDMKAMDSKSNGAIAWSNQTSFTCQDIFKETNATYP | |
| | SSDVPCDATLTEKSFETDMNLNFQNLLVIVLRILLLKVAGENLLMTLRLWSSRAKRSGSGA | |
| | TNFSLLKQAGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MATRLLCWAALCLLGAELTEAGVAQSPRYKIIEKRQSVAFWCNPISGHATLYWYQQILGQG | 336 |
| BP15TA | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGATNFSLLKQAGDVEENPGPMDWTWILFLVAAATRVHSNWVNVISDLKKIED | |
| | LIQSMHIDATLYTESDVHPSCKVTAMKCFLLELQVISLESGDASIHDTVENLIILANNSLS | |
| | SNGNVTESGCKECEELEEKNIKEFLQSFVHIVQMFINTSSGGGSGGGGSGGGGSGGGGSGG | |
| | GSLQITCPPPMSVEHADIWVKSYSLYSRERYICNSGFKRKAGTSSLTECVLNKATNVAHWT | |
| | TPSLKCIRDPALVHQRPAPPSTVTTAGVTPQPESLSPSGKEPAASSPSSNNTAATTAAIVP | |
| | GSQLMPSKSPSTGTTEISSHESSHGTPSQTTAKNWELTASASHQPPGVYPQGHSDTTVAIS | |
| | TSTVLLCGLSAVSLLACYLKSRQTPPLASVEMEAMEALPVTWGTSSRDEDLENCSHHLRAK | |
| | RSGSGEGRGSLLTCGDVEENPGPMESFLGGVLLILWLQVDWVKSQKIEQNSEALNIQEGKT | |
| | ATLTCNYTNYSPAYLQWYRQDPGRGPVFLLLIRENEKEKRKERLKVTFDTTLKQSLFHITA | |
| | SQPADSATYLCALDIYPHDMRFGAGTRLTVKPNIQNPEPAVYQLKDPRSQDSTLCLFTDED | |
| | SQINVPKTMESGTFITDKCVLDMKAMDSKSNGAIAWSNQTSFTCQDIFKETNATYPSSDVP | |
| | CDATLTEKSFETDMNLNFQNLLVIVLRILLLKVAGENLLMTLRLWSS | |
| Plasmid | MATRLLCWAALCLLGAELTEAGVAQSPRYKIIEKRQSVAFWCNPISGHATLYWYQQILGQG | 337 |
| BT15PA | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGEGRGSLLTCGDVEENPGPMDWTWILFLVAAATRVHSNWVNVISDLKKIEDL | |
| | IQSMHIDATLYTESDVHPSCKVTAMKCFLLELQVISLESGDASIHDTVENLIILANNSLSS | |
| | NGNVTESGCKECEELEEKNIKEFLQSFVHIVQMFINTSSGGGSGGGGSGGGGSGGGGSGGG | |
| | SLQITCPPPMSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTAATTAAIVPG | |
| | SQLMPSKSPSTGTTEISSHESSHGTPSQTTAKNWELTASASHQPPGVYPQGHSDTTVAIST | |
| | STVLLCGLSAVSLLACYLKSRQTPPLASVEMEAMEALPVTWGTSSRDEDLENCSHHLRAKR | |
| | SGSGATNFSLLKQAGDVEENPGPMESFLGGVLLILWLQVDWVKSQKIEQNSEALNIQEGKT | |
| | ATLTCNYTNYSPAYLQWYRQDPGRGPVFLLLIRENEKEKRKERLKVTFDTTLKQSLFHITA | |
| | SQPADSATYLCALDIYPHDMRFGAGTRLTVKPNIQNPEPAVYQLKDPRSQDSTLCLFTDED | |
| | SQINVPKTMESGTFITDKCVLDMKAMDSKSNGAIAWSNQTSFTCQDIFKETNATYPSSDVP | |
| | CDATLTEKSFETDMNLNFQNLLVIVLRILLLKVAGENLLMTLRLWSS | |

For control purposes, three additional SB transposon plasmids were prepared. Plasmid 15 contains a monocistronic expression cassette, Cassette 15, encoding mb15. Plasmid APB contains abicistronic expression cassette, Cassette APB, encoding TCRα (5′) and TCRβ (3′) with an intervening fP2A element. Plasmid BPA contains a bicistronic expression cassette, Cassette BPA, encoding TCRβ (5′) and TCRα (3′) with an intervening fP32A element. These expression cassettes, including suitable transcriptional regulatory elements, were inserted between the ITRs of SB transposon plasmids. The 5′ to 3′ order of elements in the ORF of each control expression cassette and SB Plasmid is shown in Table E4, and schematics of the ORFs of these three expression cassettes are shown in FIG. 2B.

TABLE E4
Control SB transposon plasmids.

| Plasmid Name | Cassette Name | Order of Elements (5′ to 3′) |
|---|---|---|
| Plasmid 15 | Cassette 15 | mbIL15 |
| Plasmid APB | Cassette APB | TCRα-fP2A-TCRβ |
| Plasmid BPA | Cassette BPA | TCRβ-fP2A-TCRα |

A plasmid encoding SB11 transposase, Plasmid TA, was also constructed.

### 6.2 Example 2: Generation and Evaluation of T Cells

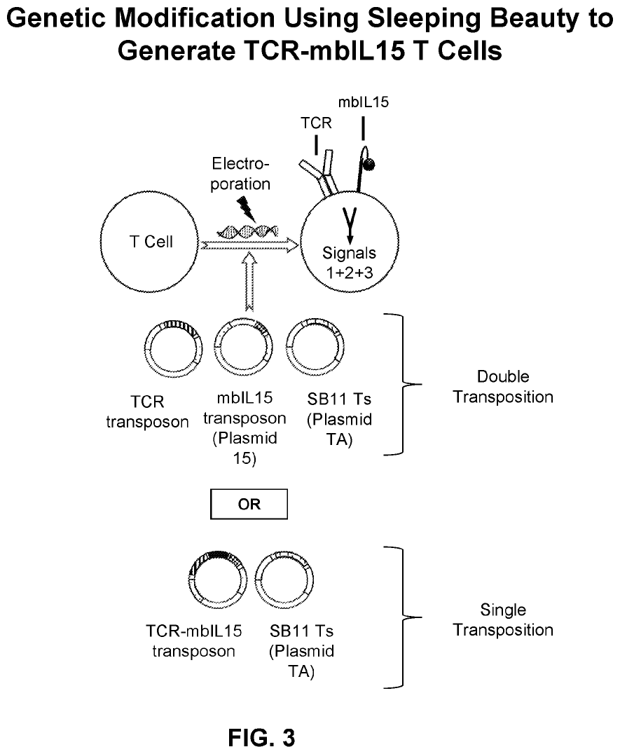

This Example describes the generation and evaluation of T cells co-expressing TCRα, TCRβ, and mbIL15 from the plasmids described in Example 1. A schematic of the gene transfer process for both double transposition (using separate plasmids encoding TCRα/TCRβ and mbIL15) and single transposition (using a tricistronic plasmid encoding TCRα/TCRβ and mbIL15 together) is shown in FIG. 3.

Briefly, peripheral blood mononuclear cells (PBMCs) were enriched from leukapheresis product obtained from a normal donor (Discovery Life Sciences, Austin, TX). The resulting PBMCs were collected, cryopreserved, and stored in the vapor phase of a liquid nitrogen tank.

To generate the TCR-T cells described in this Example 2, the plasmids described in Example 1 were electroporated into the enriched PBMCs. Briefly, cryopreserved PBMCs were thawed, resuspended in supplemented media, and incubated in a 37° C./5% CO₂ incubator for one hour. The PBMC test articles listed in Table E5 were then prepared.

TABLE E5
PBMC test articles.

| Group | Name | Description |
|---|---|---|
| 1 | NT cells | Non-transposed PBMCs |
| 2 | mbIL15 cells | PBMCs transposed with Plasmid 15 and Plasmid TA |
| 3 | APB cells | PBMCs transposed with Plasmid APB and Plasmid TA |
| 4 | BPA cells | PBMCs transposed with Plasmid BPA and Plasmid TA |
| 5 | APB+15 cells | PBMCs transposed with Plasmid APB, Plasmid 15, and Plasmid TA |
| 6 | BPA+15 cells | PBMCs transposed with Plasmid BPA, Plasmid 15, and Plasmid TA |
| 7 | APBT15 cells | PBMCs transposed with Plasmid APBT15 and Plasmid TA |
| 8 | ATBP15 cells | PBMCs transposed with Plasmid ATBP15 and Plasmid TA |
| 9 | AP15TB cells | PBMCs transposed with Plasmid AP15TB and Plasmid TA |
| 10 | AT15PB cells | PBMCs transposed with Plasmid AT15PB and Plasmid TA |
| 11 | BPAT15 cells | PBMCs transposed with Plasmid BPAT15 and Plasmid TA |
| 12 | BTAP15 cells | PBMCs transposed with Plasmid BTAP15 and Plasmid TA |
| 13 | BP15TA cells | PBMCs transposed with Plasmid BP15TA and Plasmid TA |
| 14 | BT15PA cells | PBMCs transposed with Plasmid BT15PA and Plasmid TA |

Test articles were prepared as follows:

Group 1: Rested cells were harvested, spun down, resuspended in supplemented media, and incubated in a 37° C./5% CO₂ incubator overnight.

Groups 2-14: Rested cells were harvested, spun down, resuspended in electroporation buffer together with the plasmids listed in Table E5, and electroporated. Following electroporation, cell suspensions were collected, transferred to supplemented media, and incubated in a 37° C./5% CO₂ incubator overnight.

Within 24 hours post-electroporation (Day 1), the cells were harvested from culture, counted, and sampled by flow cytometry to determine mbIL15 and TCR transgene expression. Briefly, up to 1×10⁶ cells of each test article were stained with human Fc Block (BD Biosciences 564220) first to reduce background staining for 10 minutes at room temperature. Cell suspensions were further stained with fluorochrome conjugated antibodies (listed in Table 1) diluted in Brilliant Stain Buffer (BD Biosciences 566349) for 30 minutes at 4° C. TCR expression was detected using PerCP-Cy5.5 conjugated anti-mouse TCRβ antibody specific for the murine constant region of TCRβ. Other fluorescently conjugated antibodies used included: CD3 (Clone OKT-3), IL-15 (34559), CD8 (Clone RPA-T8), and Invitrogen violet live/Dead dye (Table E6).

TABLE E6
Fluorescently Conjugated Antibodies.

| Antibody Target | Clone | Fluorophore | Company & Cat # |
|---|---|---|---|
| Live/Dead | | Pacific Blue | Invitrogen L34955A |
| Human CD3 | OKT3 | BV510 | Biolegend 317332 |
| Human CD8 | RPA-T8 | PE-Cy7 | BD Biosciences 557746 |
| Human IL-15 | 34559 | PE | R&D Systems IC2471P |
| Mouse TCR | H57-597 | PerCP-Cy5.5 | BD Biosciences 560657 |

Cells were washed with FACS buffer (PBS, 2% FBS, 0.1% sodium azide). Data were acquired using an NovoCyte Quanteon flow cytometer system (Agilent) and analyzed with FlowJo software (version 10.7.1; TreeStar, Ashland, OR) to determine the percentage of each transgenic subpopulation (mbIL15⁺mTCR⁺, mbIL15^{neg}mTCR⁺, mbIL15⁺mTCR^{neg}, mbIL15^{neg}mTCR^{neg}) present in each test article. Unless described otherwise, transgene expression was assessed on gated cell events, singlets, viable events, and CD3+ cells.

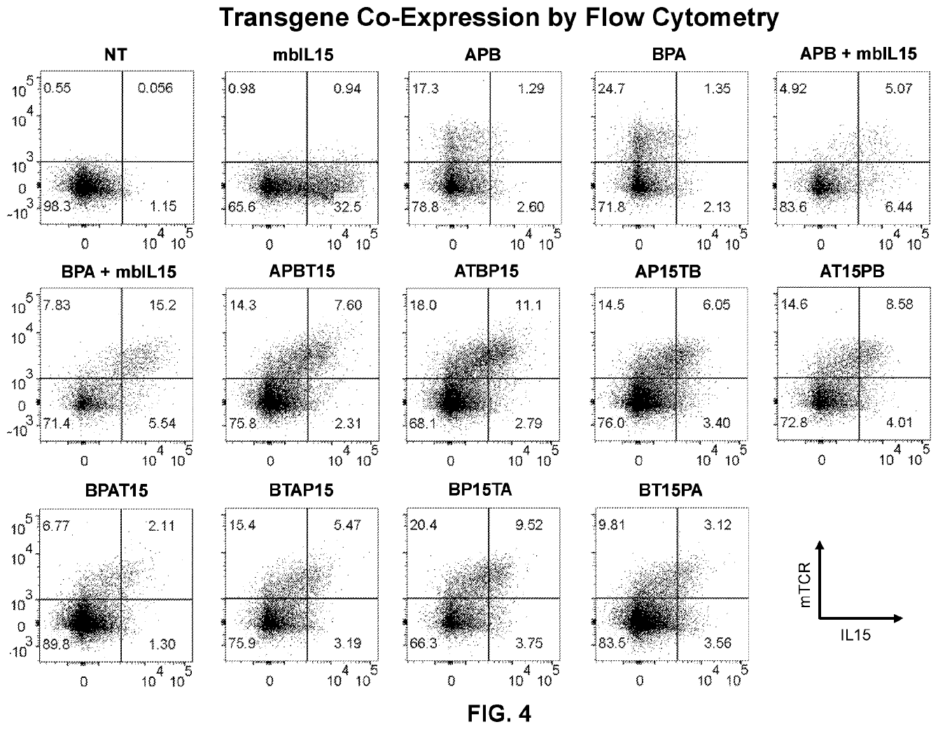

Results of flow cytometry are shown in FIG. 4 and Table E7.

TABLE E7
Day 1 post-electroporation specifications and transgene
expression of genetically modified T cells.

| | | Viability | mbIL15⁺ | mTCR⁺ | mbIL15⁺mTCR⁺ |
|---|---|---|---|---|---|
| Group | Name | (%) | (% CD3⁺) | (% CD3⁺) | (% CD3⁺) |
| 1 | NT cells | 97.5 | N/A | N/A | N/A |
| 2 | mbIL15 cells | 83.9 | 33.4 | N/A | N/A |
| 3 | APB cells | 68.3 | N/A | 18.6 | N/A |
| 4 | BPA cells | 75.4 | N/A | 26.1 | N/A |
| 5 | APB+15 cells | 65.7 | 11.5 | 9.99 | 5.07 |
| 6 | BPA+15 cells | 78.5 | 20.7 | 23.0 | 15.2 |
| 7 | APBT15 cells | 78.0 | 9.91 | 21.9 | 7.60 |
| 8 | ATBP15 cells | 82.7 | 13.9 | 29.1 | 11.1 |
| 9 | AP15TB cells | 84.1 | 9.45 | 20.6 | 6.05 |
| 10 | AT15PB cells | 81.6 | 12.6 | 23.2 | 8.58 |
| 11 | BPAT15 cells | 64.3 | 3.41 | 8.88 | 2.11 |
| 12 | BTAP15 cells | 73.8 | 8.66 | 20.9 | 5.47 |
| 13 | BP15TA cells | 83.7 | 13.3 | 29.9 | 9.52 |
| 14 | BT15PA cells | 74.5 | 6.68 | 12.9 | 3.12 |

### 6.3 Example 3: Generation and Evaluation of Expanded T Cells

This Example describes the generation and evaluation of T cells co-expressing TCRα, TCRβ, and mbIL15 from the plasmids described in Example 1. TCR-T cells described in this Example 3 were generated similarly to those described in Example 2 except as indicated below.

Briefly, cryopreserved PBMCs were thawed, resuspended in supplemented media (IL-7+IL-15), and incubated in a 37° C./5% CO₂ incubator for one hour.

Test articles as listed above in Table E5 were then prepared as follows:

Group 1: Rested cells were harvested, spun down, resuspended in recovery media (50:50 media containing IL-7+IL-15+n-acetylcysteine (NAC)), and incubated in a 37° C./5% CO₂ incubator overnight.

Groups 2-14: Rested cells were harvested, spun down, resuspended in electroporation buffer together with the plasmids listed in Table E5, and electroporated. Following electroporation, cell suspensions were collected, transferred to recovery media (50:50 media containing IL-7+IL-15+NAC), and incubated in a 37° C./5% CO₂ incubator overnight.

Groups 3-14: Within 24 hours post-electroporation (Day 1), mTCR positive (mTCR+) cells were isolated using mTCR antibody and MACS® Cell Separation system (Miltenyi Biotec). Live cells from groups 1 & 2 and live TCR+ enriched cells from groups 3-14 were transferred to G-REX® culture plates (Wilson Wolf Manufacturing) and incubated with a first expansion media (50:50 media containing IL-21+IL-7) with irradiated allogeneic feeder cells and OKT3 antibody. Cells were fed regularly with cytokines. After 13 days of first phase expansion, cells were harvested, and expression of mTCR and mbIL15 was detected on CD3+ gated population with mouse TCR beta antibody and IL-15 antibody as described in Example 2. Cell count and viability was accessed with a NC3000 cell counter. Unless described otherwise, transgene expression was assessed on gated cell events, singlets, viable events, and CD3+ cells.

Expression of mTCR and mbIL15 and cell viability was assessed for each test article at two separate time points: 1) after electroporation (Day 1), and 2) after first expansion phase (Day 13).

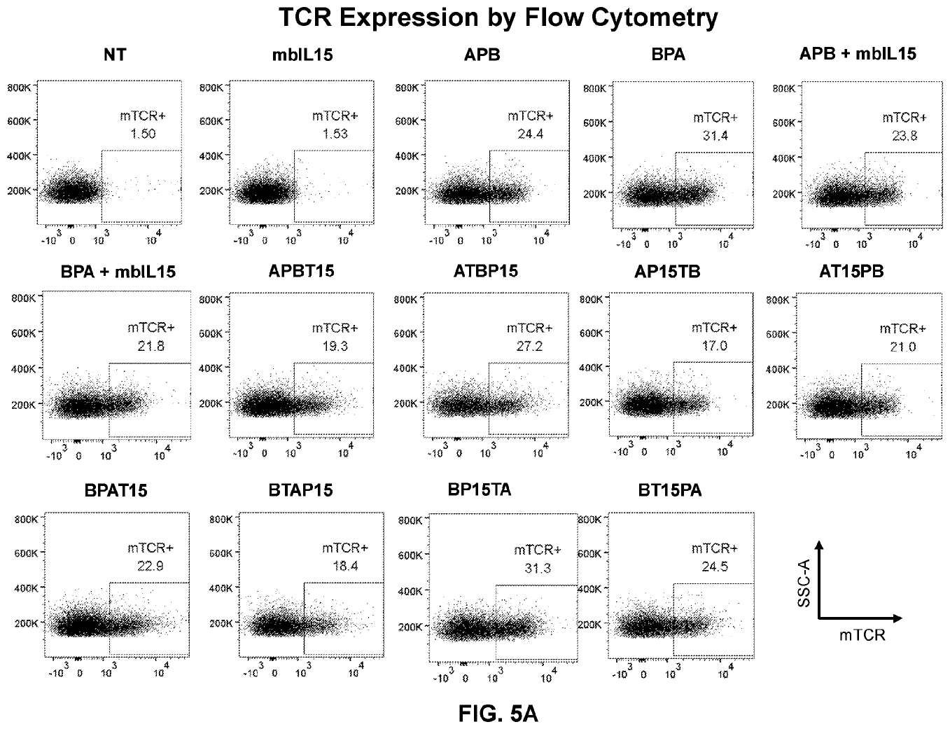

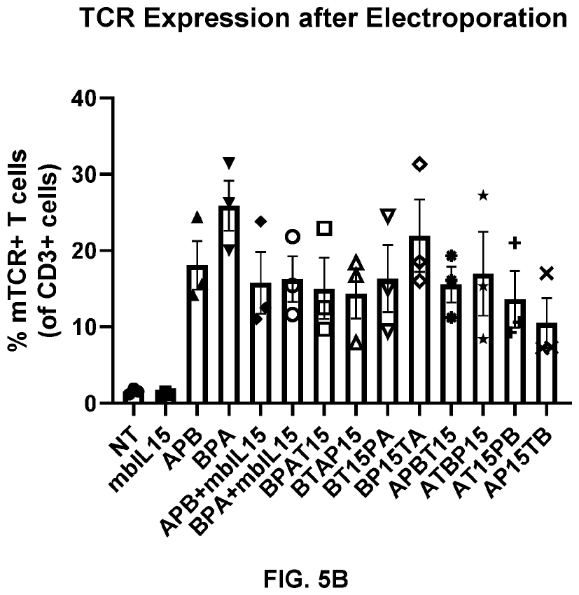

TCR expression after electroporation (Day 1) is shown in FIG. 5A-5B. FIG. 5A provides representative TCR expression data from each test article. FIG. 5B provides TCR expression data from three donors presented as % mTCR+ cells out of CD3+ cells.

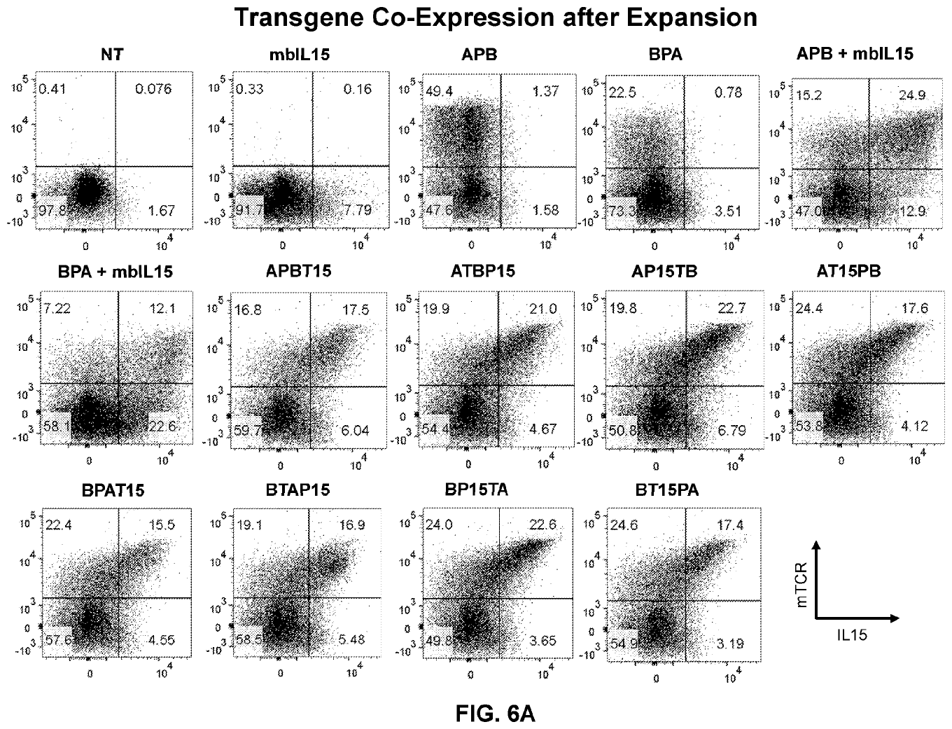

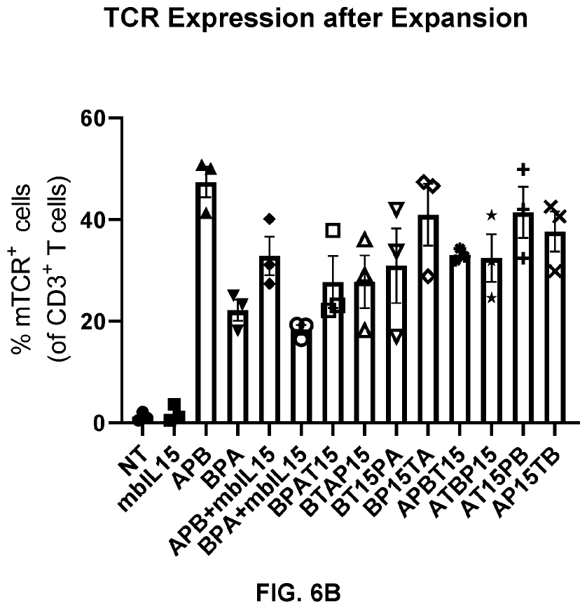

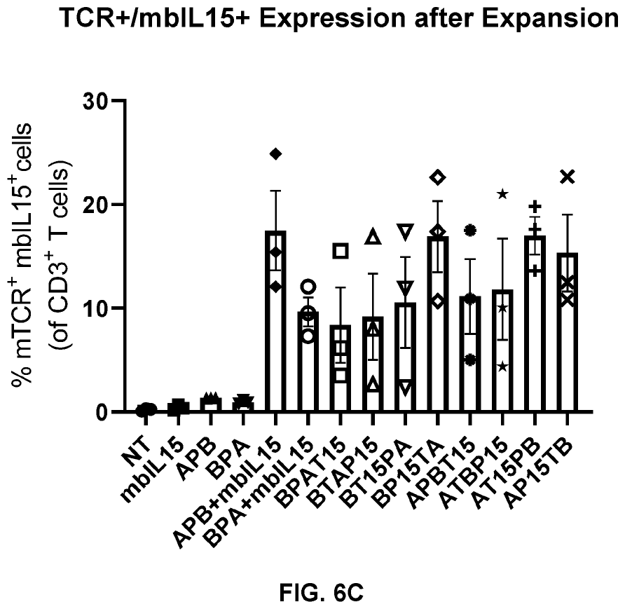

TCR and mbIL15 expression after first phase expansion (Day 13) is shown in FIG. 6A-6C. FIG. 6A provides representative TCR and mbIL15 expression data from each test article. FIG. 6B provides TCR expression data from three donors presented as % mTCR+ cells out of CD3+ cells and FIG. 6C provides % TCR+mbIL15+ cells out of CD3+ cells.

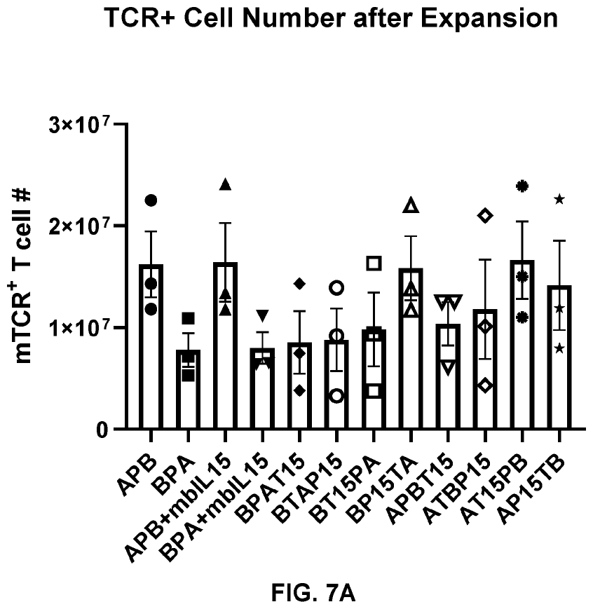

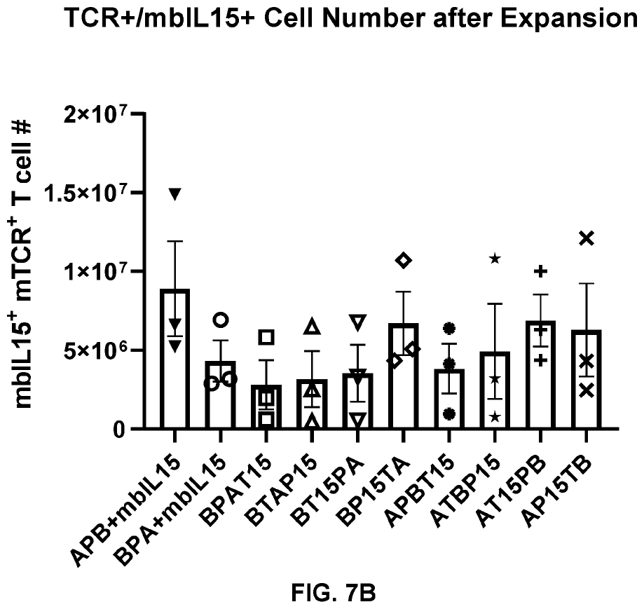

TCR+ and TCR+mbIL15+ cell number was also assessed after first phase expansion (Day 13) as shown in FIG. 7A-7B. FIG. 7A provides TCR expression data from three donors presented as total number of mTCR+ T cells and FIG. 7B provides total number of TCR+mbIL15+ T cells.

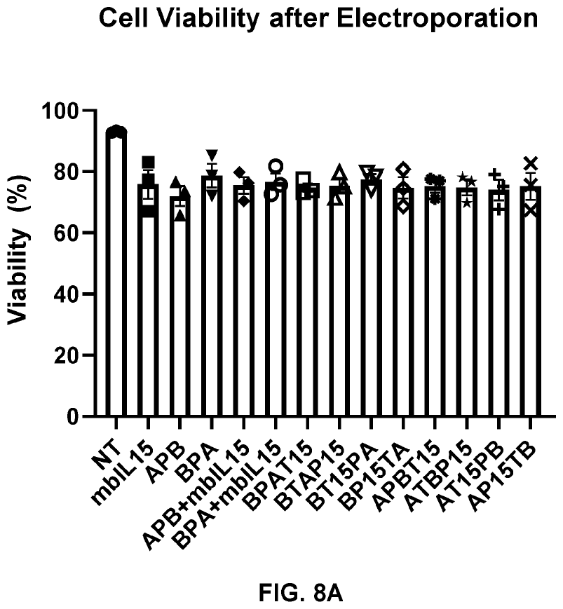

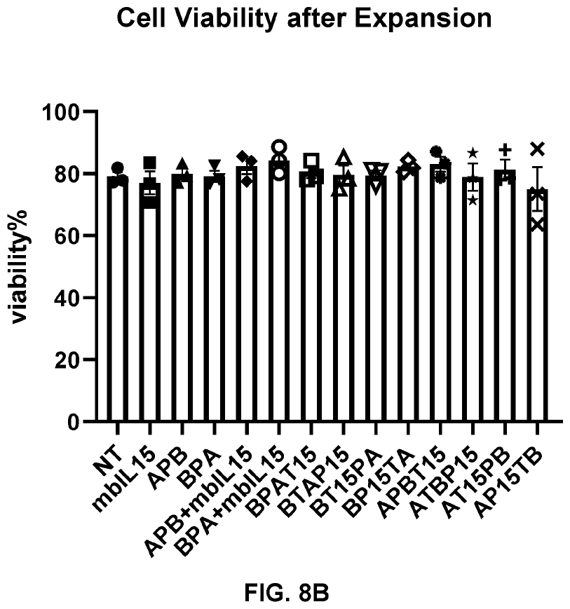

Cell viability after electroporation (Day 1) and after first phase expansion (Day 13) is shown in FIGS. 8A & 8B, respectively.

The transgene expression data and cell count data demonstrate that BP15TA and AP15 TB are the most potent candidates to have mbIL15+ TCR+ T cells with the highest level of TCR and mbIL15 expression. Viability data demonstrated that despite of the size of the tricistronic mbIL15+ TCR vectors (Groups 7-14), the viability is similar to the two-vector co-transfection system (Groups 5 & 6).

Functionality of the TCR-T cells was also measured following first phase expansion (Day 13) as described below.

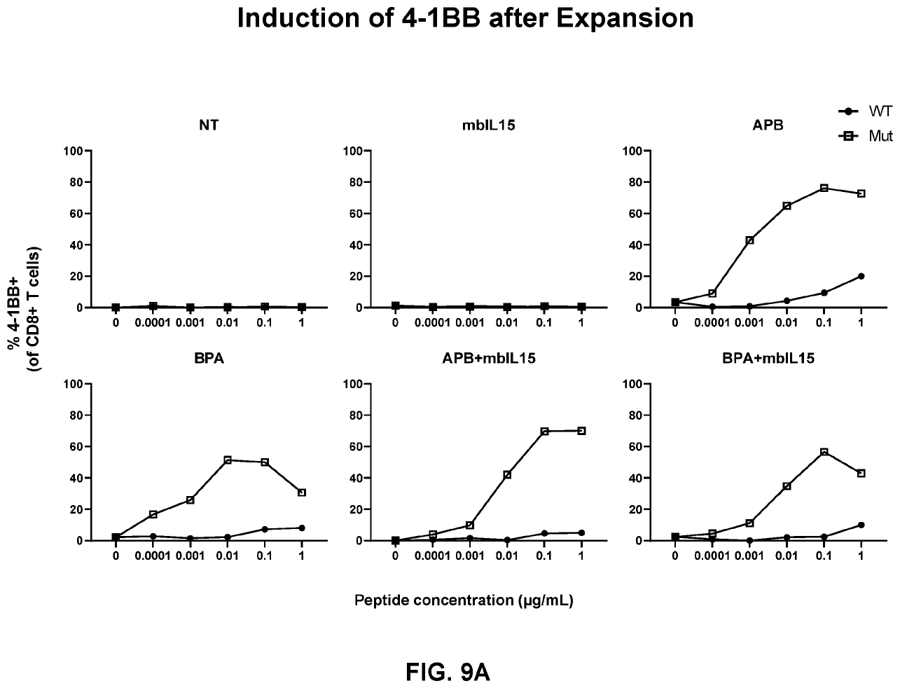

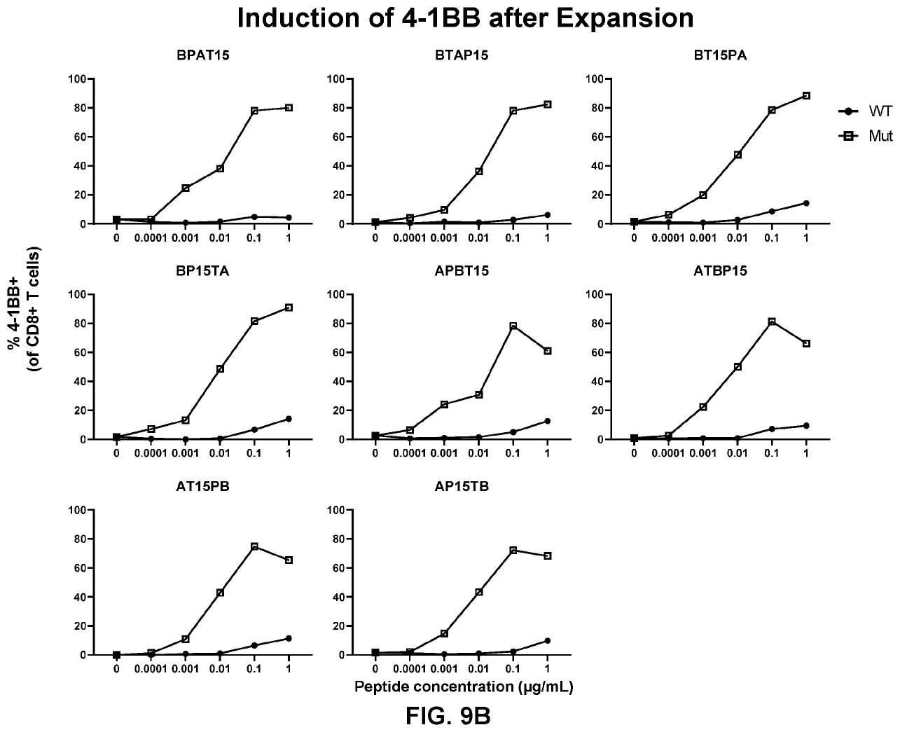

Activation of TCR-T cells generated by electroporation with different polycistronic plasmids was assessed. After 13 days of first phase expansion, cells were co-cultured with wild-type or mutant neoantigen peptide pulsed T2 cells which have endogenous expression of HLA-A*02:01. After overnight incubation, cells were harvested and induction of 4-1BB molecule on CD3+CD8+ cells was detected with 4-1BB antibody. Results are shown in FIG. 9A-9B demonstrating that mbIL15/TCR-T cells were highly avid and specific to the target neoantigen as measured by upregulation of 4-1BB co-stimulatory receptor with negligible recognition of wild type sequence. There was no significant difference in function between the mbIL15/TCR-T cells generated using different polycistronic plasmids.

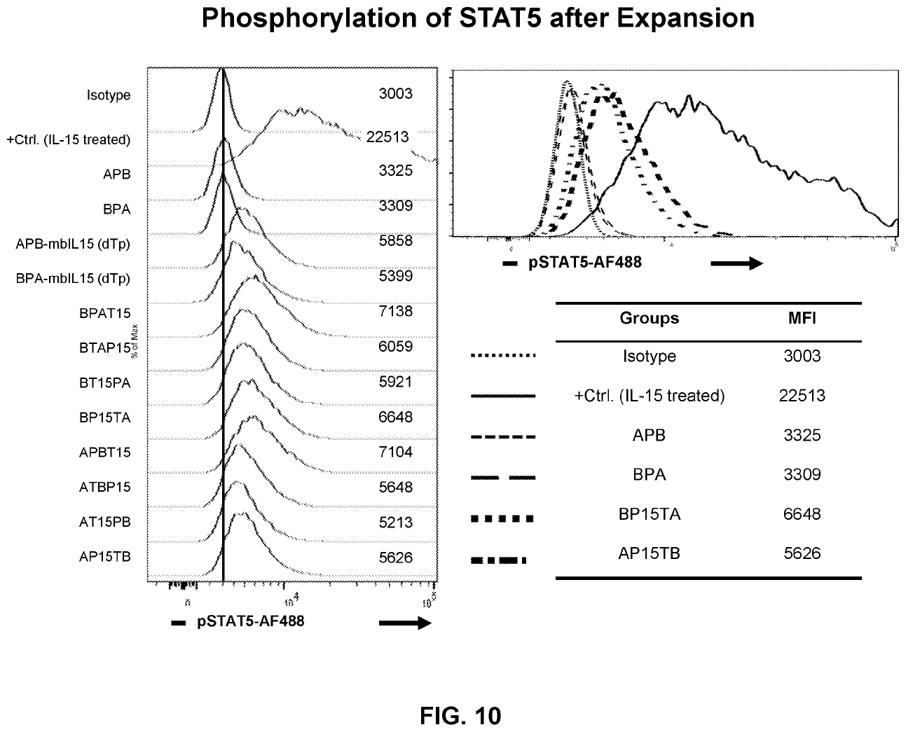

The level of phosphorylated STAT5 was also assessed for TCR-T cells electroporated with different polycistronic plasmids. After 13 days of first phase expansion, cells were washed and incubated in cytokine-free 50:50 media overnight to stabilize the phosphorylation of STAT5. Phosphorylation of STAT5 was detected the following day on CD3+ cells using pSTAT5 (pY694). Results are shown in FIG. 10 demonstrating that the expressed mbIL15 is functional. IL15 signaling was activated, inducing phosphorylation of STAT5 (downstream of IL15 receptor). Phosphorylation of STAT5 in mbIL15 TCR-T cells generated with different polycistronic plasmids was not significantly different between plasmids but was increased in all mbIL15 containing plasmids relative to TCR only conditions that lacked mbIL15.

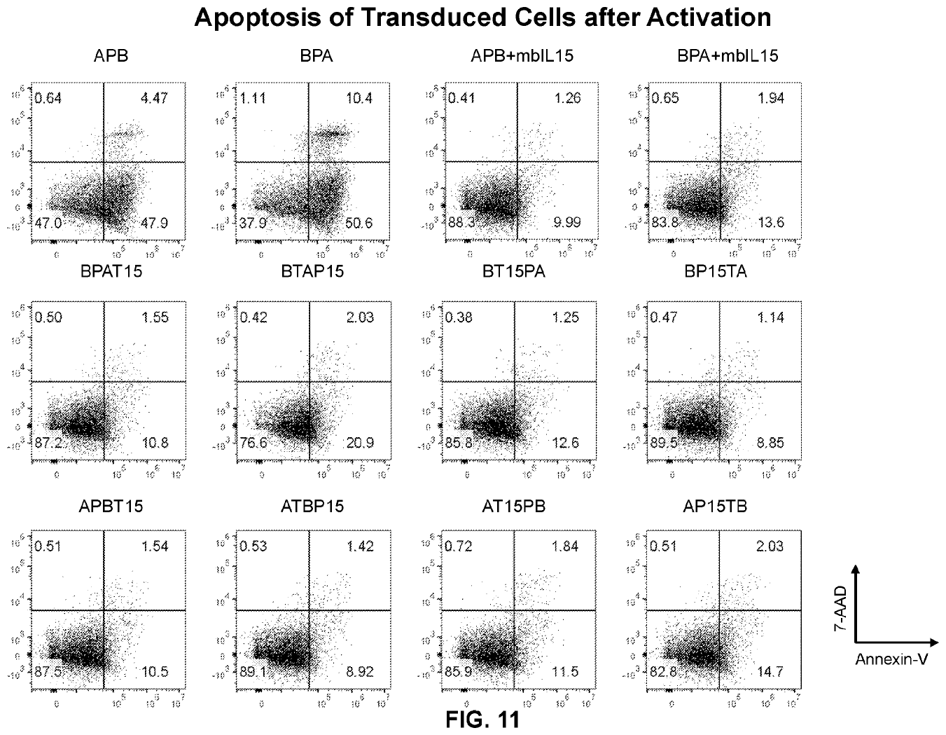

The level of apoptosis after 9 days of activation was assessed for TCR-T cells electroporated with different polycistronic plasmids. After 13 days of first phase expansion, cells were washed and activated with CD3/CD28 Dynabeads® (ThermoFisher) for 9 days. After activation, apoptosis of CD3+ TCR+ cells was monitored with AnnexinV/7ADD kit (Biolegend). Results are shown in FIG. 11 demonstrating that expression of mbIL15 on CD3+ TCR+ cells inhibited AICD (activation-induced cell death) as measured by fewer cells stained for AnnexinV and/or PI. This inhibition of AICD was not significantly different between the different polycistronic plasmids tested, nor was it different from two-vector systems (APB+mbIL15 and BPA+mbIL15).

A second expansion phase was performed as described below and vector copy number (VCN) following the second expansion phase was assessed. Briefly, T cells from Groups 3-14 were isolated by MACS using mTCR antibodies. T cells from Groups 2-14 were then incubated with a second expansion media (50:50 media containing IL-21) and irradiated feeder cells and OKT3 antibody. Cells were fed regularly with cytokines. After 15 days second phase expansion, cells were harvested and VCN was detected using qPCR as average number of Sleeping Beauty transgene DNA copy per cell in a sample. Results are shown in Table E8 demonstrating that low levels of vector were detected in TCR-T cells and mbIL15/TCR-T cells after two rounds of expansion.

TABLE E8
Vector Copy Number (VCN) after second expansion phase.

| Group | Name | VCN |
|---|---|---|
| 2 | mbIL15 cells | 0.3 |
| 3 | APB cells | 0.4 |
| 4 | BPA cells | 1.4 |
| 5 | APB+15 cells | 2.6 |
| 6 | BPA+15 cells | 2.1 |
| 7 | APBT15 cells | 4.7 |
| 8 | ATBP15 cells | 4.5 |
| 9 | AP15TB cells | 5.3 |
| 10 | AT15PB cells | 3.9 |
| 11 | BPAT15 cells | 1.5 |
| 12 | BTAP15 cells | 2.1 |
| 13 | BP15TA cells | 2.3 |
| 14 | BT15PA cells | 2.9 |

Conclusion: The series of data described in this example illustrate that BP15TA and AP15 TB are the most potent candidates to generate mbIL15 TCR-T cell with the highest level of TCR and mbIL15 expression. All plasmids evaluated expressing mbIL15 increased phosphorylation of STAT5 indicating the mbIL15 expressed at sufficient levels in T cells to elicit downstream signaling. Moreover, co-expression of mbIL15 with a transgenic TCR, reduces AICD following T cell activation. Furthermore, all tricistronic mbIL15/TCR plasmids tested resulted in acceptable VCN values.

### 6.4 Example 4: Evaluation of Polycistronic TCR Constructs with Different Murine Constant Regions

This Example evaluates the effect of different murine constant regions on the TCR constructs described above in Examples 1-3.

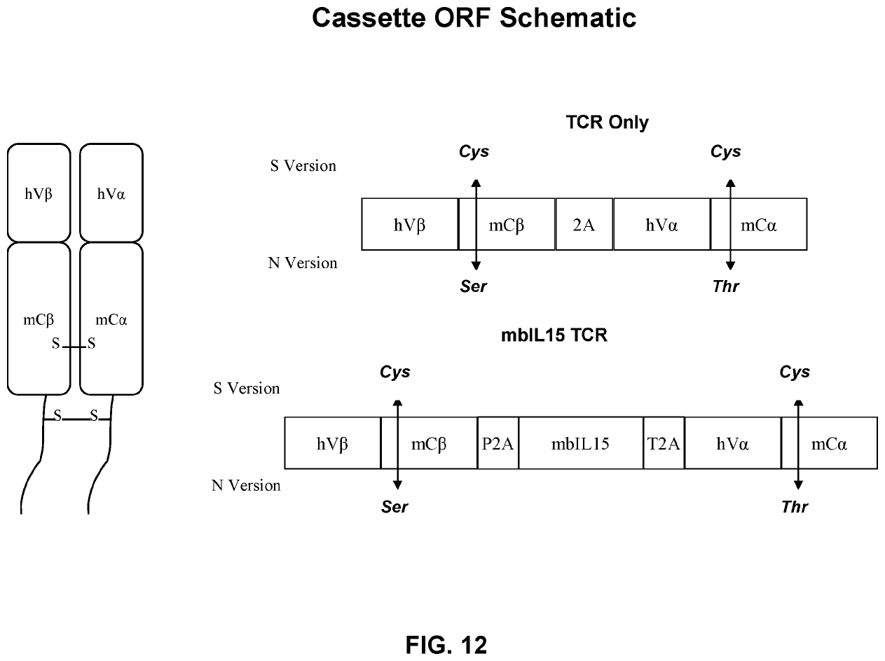

Briefly, the amino acid sequences of the TCRα chain and TCR3 chain examined here are identical to the TCRα chain and TCR chain described in Examples 1-3 except that the constant region of each chain is not cysteine-substituted. Specifically, the TCRα chain was generated by fusing a human Vα region, including its N-terminal signal sequence (SEQ ID NO: 1006) with a glutamic acid at position 2, to a murine Cα region modified by substituting a leucine at amino acid position 112, an isoleucine at amino acid position 114, and a valine at amino acid position 115 (SEQ ID NO: 42). The TCR3 chain was generated by fusing a human Vβ region, including its N-terminal signal sequence (SEQ ID NO: 2006) with an alanine at position 2, to a murine wild-type Cβ (SEQ ID NO: 52). The constructs containing the cysteine-substituted constant domains, as described in Examples 1-3, are referred to below as the “S version” and the newly-generated constructs containing the non-cysteine-substituted constant domains are referred to below as the “N version”. A schematic of these constructs is provided in FIG. 12.

The unified plasmids, “NU version” referred to below, vary in the nucleotide sequence of the TCR constant regions compared to the “N version”. All “NU versions” contain the same nucleotide sequences encoding the TCR constant regions (Cα and Cβ); however, the amino acid sequences of the TCR constant regions encoded by the “NU version” are identical to those of the “N version.” No other differences exist between the “N version” and “NU version.”

To generate the TCR-T cells described in this Example 4, the plasmids described above were electroporated into the enriched PBMCs. Briefly, cryopreserved PBMCs were thawed, exchanged into 50:50 media and electroporated. The PBMC test articles listed in Table E9 were then prepared. Where it is indicated that cells were transposed, cells were co-electroporated with the indicated plasmid and plasmid TA.

TABLE E9
PBMC test articles.

| Group | Name | Description |
|---|---|---|
| 2.1 | NT cells | Non-transposed PBMCs |
| 2.2 | BPA | PBMCs transposed with |
| | cells | Plasmid BPA |
| 2.3 | BPA-N | PBMCs transposed with N |
| | cells | version of Plasmid BPA |
| 2.4 | AP15TB | PBMCs transposed with |
| | cells | Plasmid AP15TB |
| 2.5 | BP15TA | PBMCs transposed with |
| | cells | Plasmid BP15TA |
| 2.6 | AP15TB-N | PBMCs transposed with N |
| | cells | version of Plasmid AP15TB |
| 2.7 | BP15TA-N | PBMCs transposed with N |
| | cells | version of Plasmid BP15TA |
| 2.8 | AP15TB-NU | PBMCs transposed with unified |
| | cells | N version of Plasmid AP15TB |
| 2.9 | BP15TA-NU | PBMCs transposed with unified |
| | cells | N version of Plasmid BP15TB |

Test articles were prepared as follows:

Group 2.1: Cells were harvested, spun down, resuspended in recovery media (50:50 media containing IL-7+IL-15+n-acetylcysteine (NAC)), and incubated in a 37° C./5% CO₂ incubator overnight.

Groups 2.2-2.9: Cells were harvested, spun down, resuspended in electroporation buffer together with the plasmids listed in Table E9, and electroporated. Following electroporation, cell suspensions were collected, transferred to recovery media (50:50 media containing IL-7+IL-15+NAC), and incubated in a 37° C./5% CO₂ incubator overnight.

Within 24 hours post-electroporation (Day 1), live cells were transferred to G-REX® culture plates (Wilson Wolf Manufacturing) and incubated with a first expansion media (50:50 media containing IL-21+IL-7+IL-12+ T Cell TransAct™). Cells were fed regularly with cytokines. After 11 days of first phase expansion, TCR+ cells were isolated with mTCR antibody. The isolated TCR+ T cells were transferred to G-REX® culture plates (Wilson Wolf Manufacturing) and incubated with a second expansion media (50:50 media containing 3000 IU/ml of IL-2+ T Cell TransAct™). Cells were fed regularly with cytokines. After 11 or 16 days of second phase expansion, cells were harvested, and the various assays described below were performed.

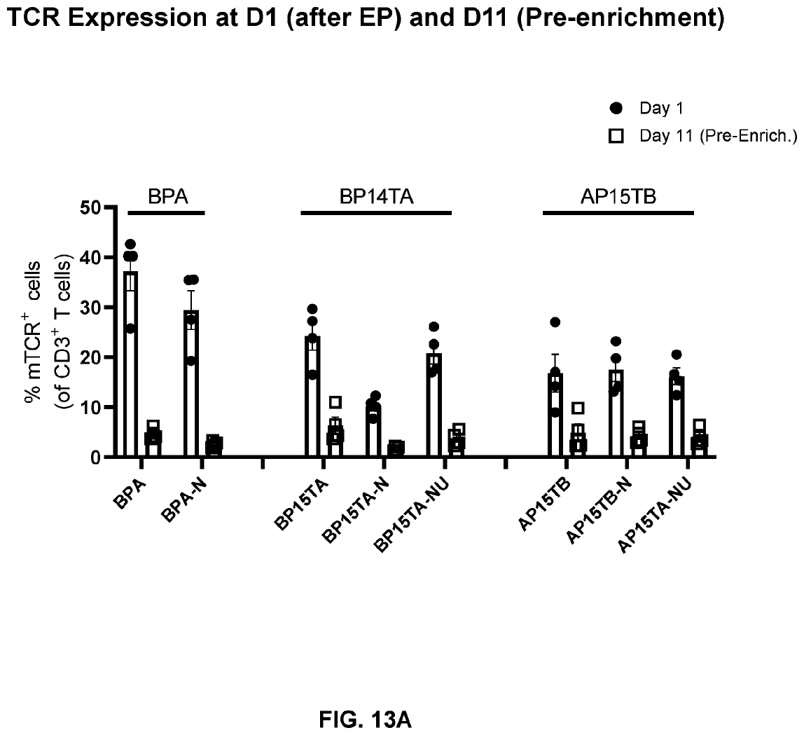

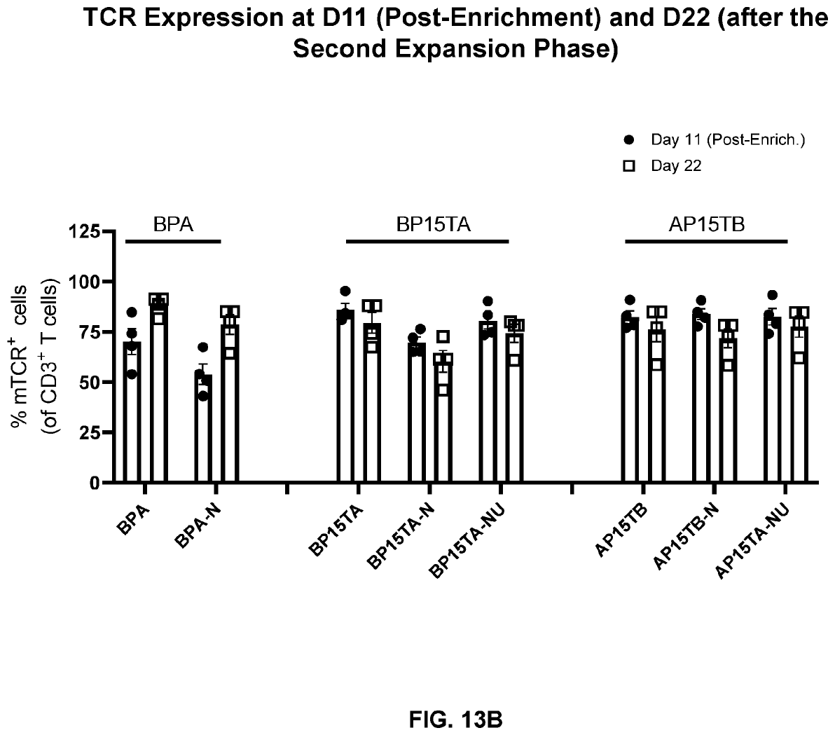

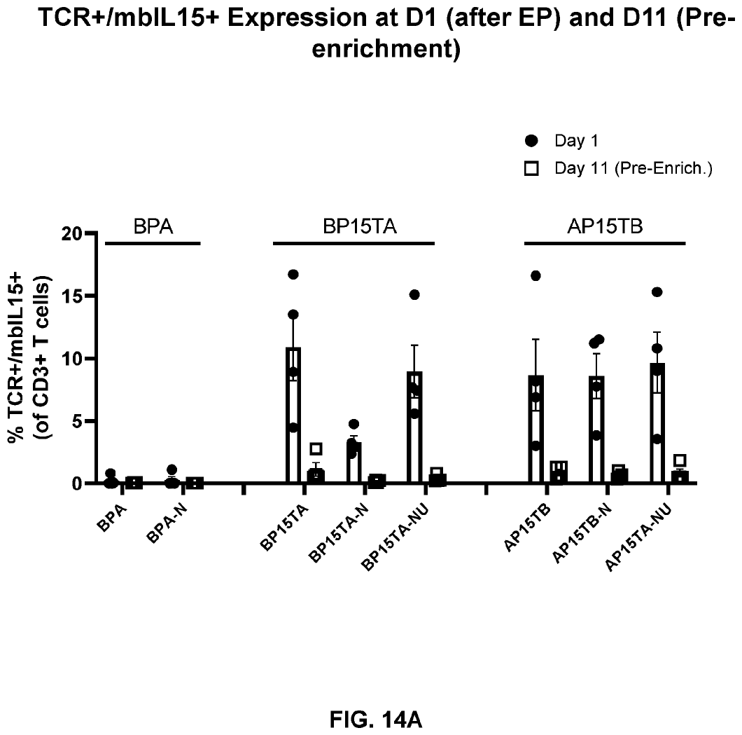

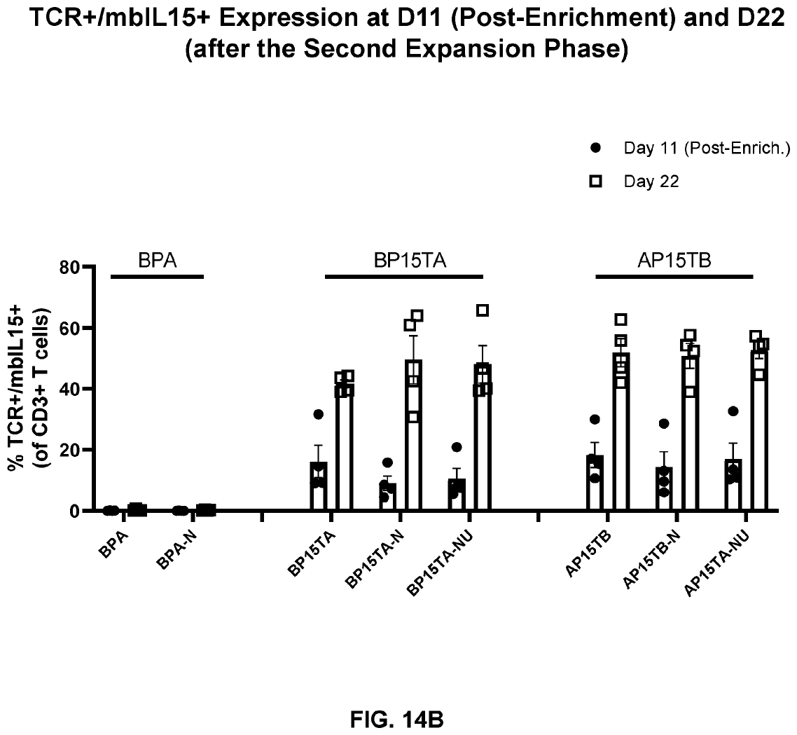

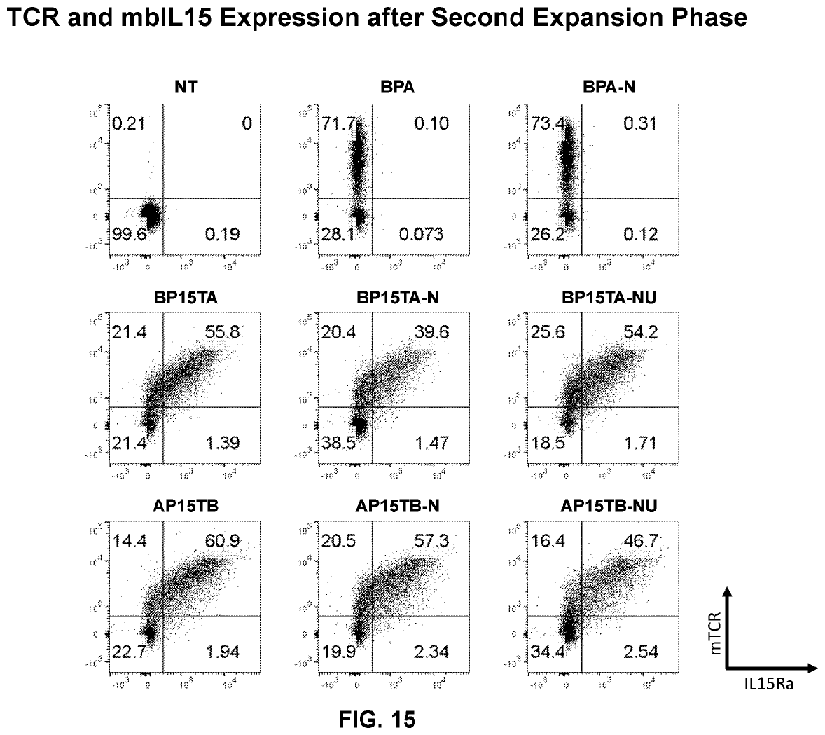

Transgene expression was assessed for T cells electroporated with different polycistronic plasmids. On Day 1 (post-electroporation), Day 11 pre-enrichment (post-1′ phase expansion), Day 11 post-enrichment, and Day 22 (post-2^{nd} phase expansion), cells were harvested and the expression of mTCR and mbIL15 was detected on CD3+ gated population with mouse TCR beta antibody and IL-15Rα antibody. On Day 1 after electroporation the level of TCR expression was similar between the different versions of polycistronic plasmids (FIG. 13A). Prior to enrichment on Day 11, TCR expression trended lower in mbIL15/TCR T cells compared to TCR only; however, TCR expression was comparable post-enrichment on Day 11 (FIG. 13B). On Day 22, when the cells were harvested, TCR expression was comparable with no notable difference between “S” and “N” versions of the polycistronic plasmids. Co-expression of TCR and mbIL15 was found to be similar between the different versions of polycistronic plasmids throughout the process (FIGS. 14-15).

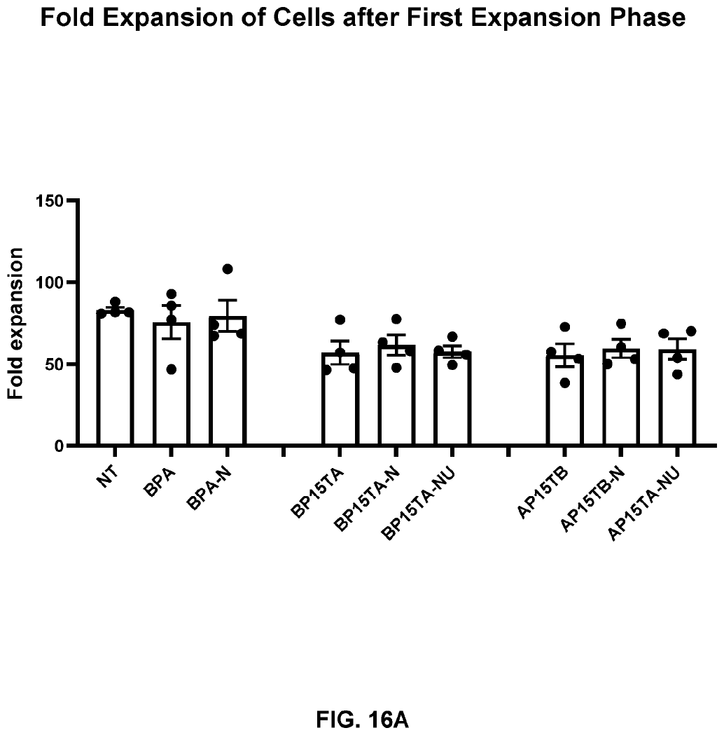

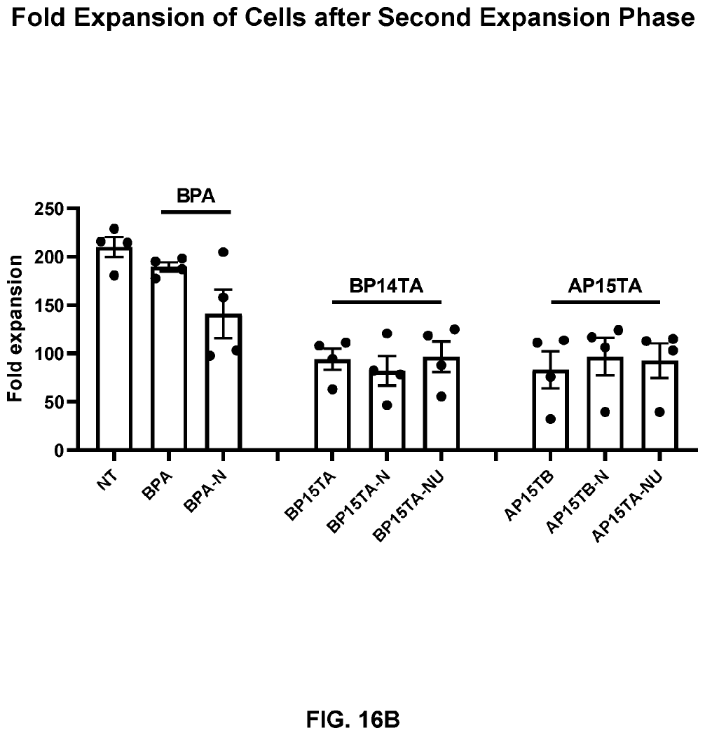

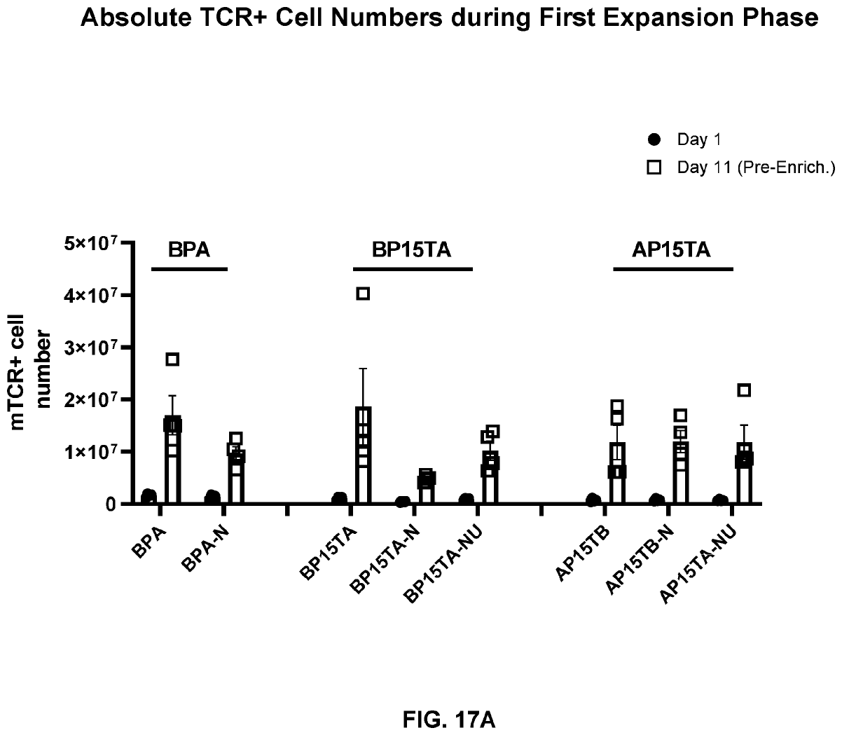

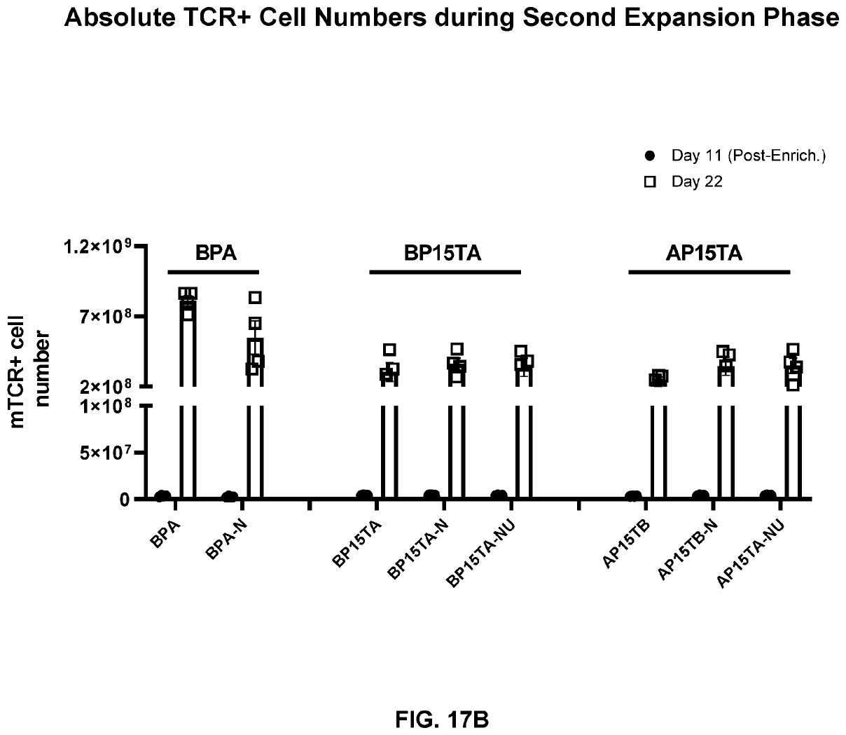

Fold expansion of total cell count (FIG. 16A-16B) and mTCR+ cell count (FIG. 17A-17B) was assessed for T cells electroporated with different polycistronic plasmids. Fold expansion value was calculated as: Cell number on Day 11/Cell number on Day 1 (FIG. 16A) and Cell number on Day 22/Cell number on Day 11 (FIG. 16B). Cells transposed with mbIL15/TCR tricistronic plasmids tended to expand less than cells transposed with TCR only bicistronic plasmids during both first and second phase expansion. However, significant degrees of expansion were achieved in all groups and no difference was seen between the different versions of the polycistronic plasmids. mTCR+ cell number was calculated as: Total cell number X CD3 population (%) X mTCR population (%).

The above transgene expression and cell growth data demonstrate that cells generated using N version and NU versions of the polycistronic plasmids were not phenotypically different from cells generated using the S version of the polycistronic plasmids.

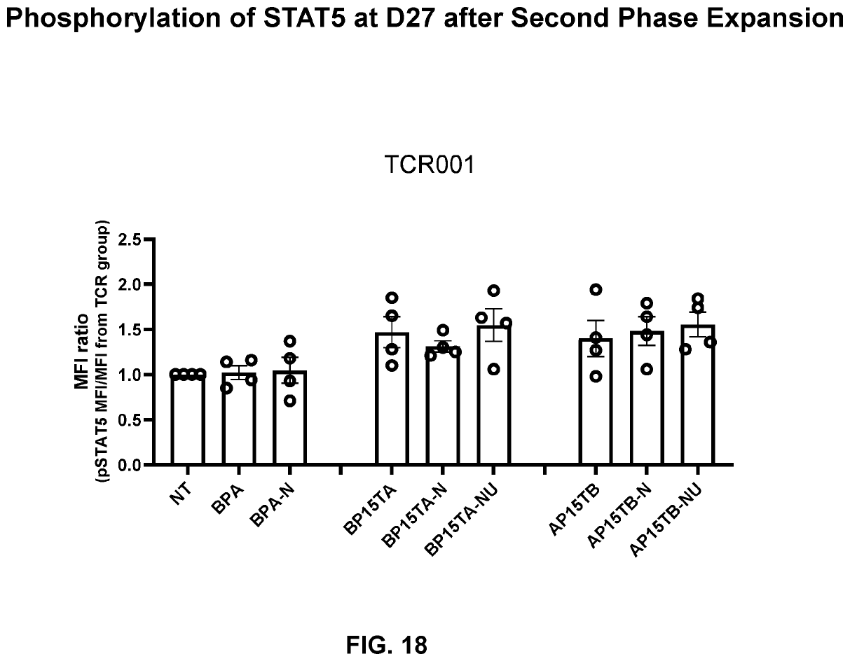

To carry out the pSTAT5 assay, the 4-1BB induction assay, and IFN-γ assay described below, the second expansion phase was extended to 16 days (due to the logistic load). Phosphorylation of STAT5 in T cells at Day 27 was detected on CD3+ cells with pSTAT5 (pY694). The pSTAT5 data shown in FIG. 18 demonstrated that the expressed mbIL15 is functional. IL15 signaling was activated, inducing phosphorylation of STAT5 (downstream of IL15 receptor). Phosphorylation of STAT5 in mbIL15 TCR-T cells generated with the different versions of polycistronic plasmids was not significantly different. The levels of phosphorylated STAT5 trended higher in cells co-expressing mbIL15 and TCR compared to those expressing TCR alone.

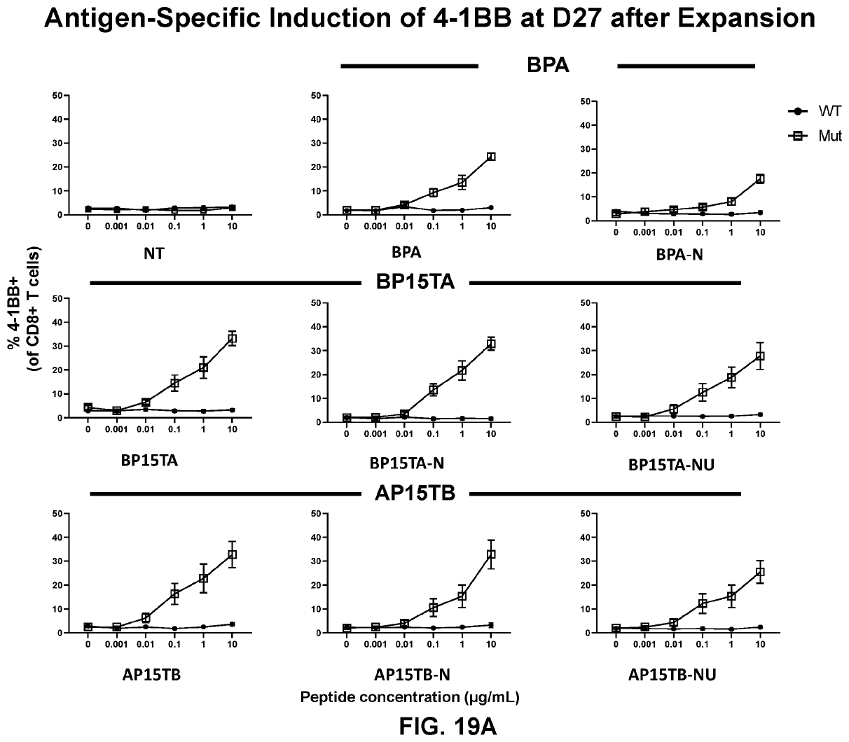

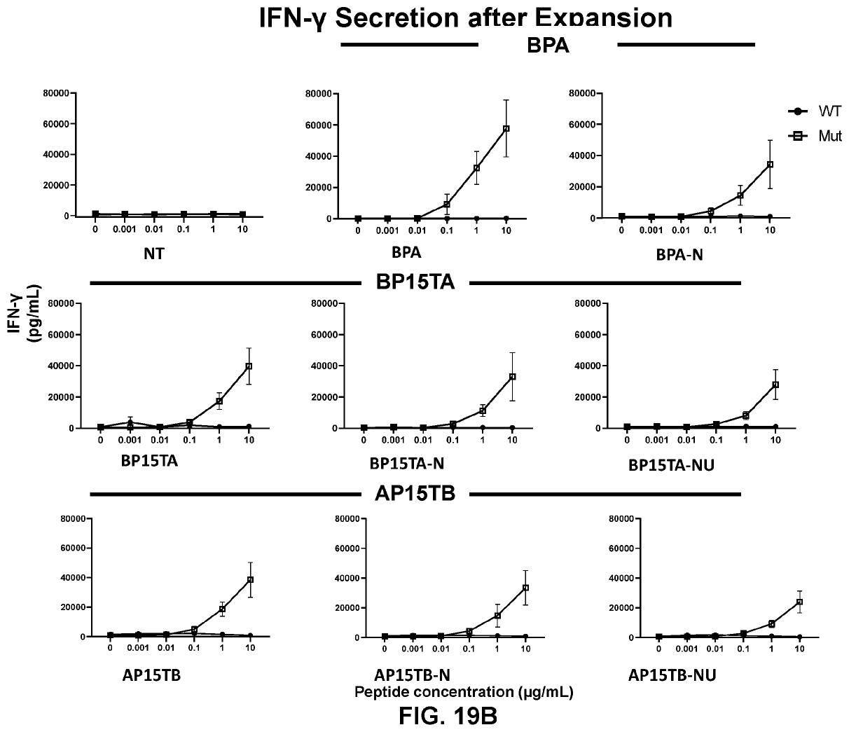

To assess antigen-specific activation of the generated TCR-T cells, overnight co-culture of the generated TCR-T cells with wild-type or mutant neoantigen pulsed DCs (HLA matched) was performed after 16 days of second phase expansion and 4-1BB induction and IFN-7 secretion were measured. Induction of 4-1BB on CD3+CD8+ cells was detected with 4-1BB antibody. Secretion of IFN-γ measured by ELISA (Clone 2G1 and B133.5, Thermo Fisher). The 4-1BB induction results shown in FIG. 19A, and IFN-γ secretion results shown in FIG. 19B demonstrate that the function of mbIL15 TCR-T cells generated with different versions of the polycistronic plasmids was not significantly different.

The long-term withdrawal (LTWD) assay was performed to examine the transgene expression, survival and activation of T cells cultured under cytokine-free conditions. The LTWD assay was performed as follows. The engineered T cells at Day 22 (post-first and second phase expansion) were transferred to T25 flask and cultured for 4 weeks in cytokine-free media (50:50). 50% of media was exchanged every week. For the control groups (groups 2.2 & 2.3, Table E9), cells were treated with 300 U/ml IL-2 twice a week while exchanging the 50% of media. Flow data were acquired using an NovoCyte Quanteon flow cytometer system (Agilent) and analyzed with FlowJo software (version 10.7.1; TreeStar, Ashland, OR). (Data n=4, pooled from 2 independent experiments)

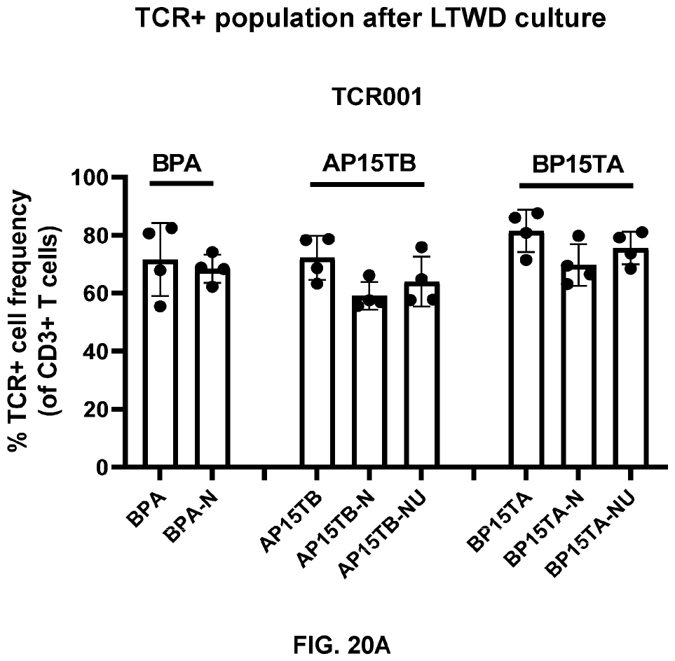

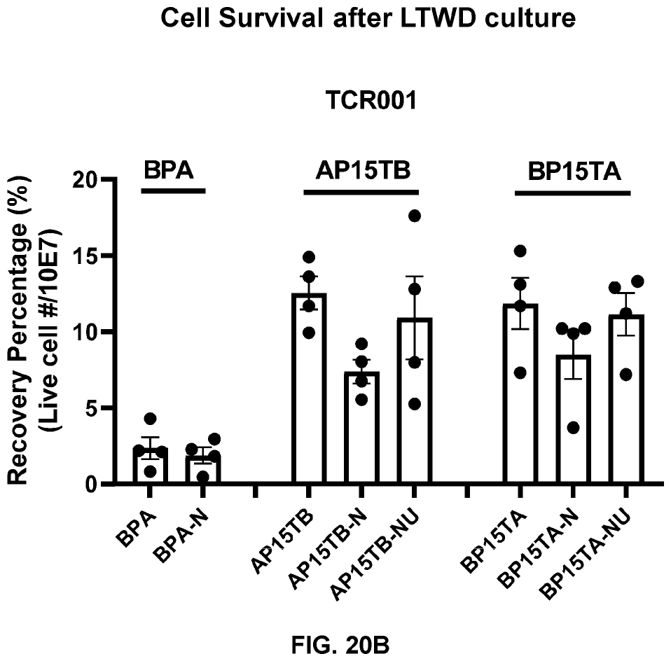

After 4 weeks LTWD incubation, the expression of mTCR was detected on CD3+ gated population with mouse TCR beta antibody (FIG. 20A) and cell count and viability were accessed (FIG. 20B). This mTCR expression and cell count data demonstrated no significant difference between mbIL15 TCR-T cells generated with different versions of the polycistronic plasmids. The number of viable cells decreased after long-term cytokine withdrawal in all groups, but cells from the groups co-expressing mbIL15 and TCR survived 5-6 fold more compared to cells from the TCR only groups.

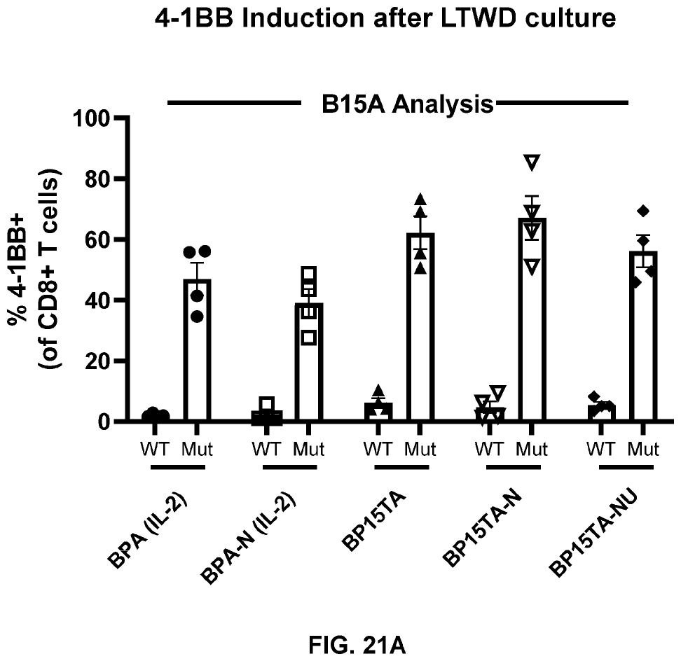

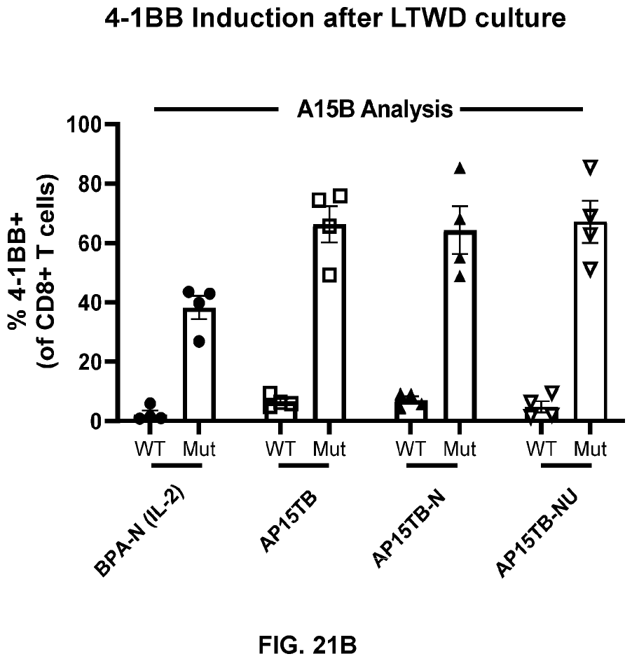

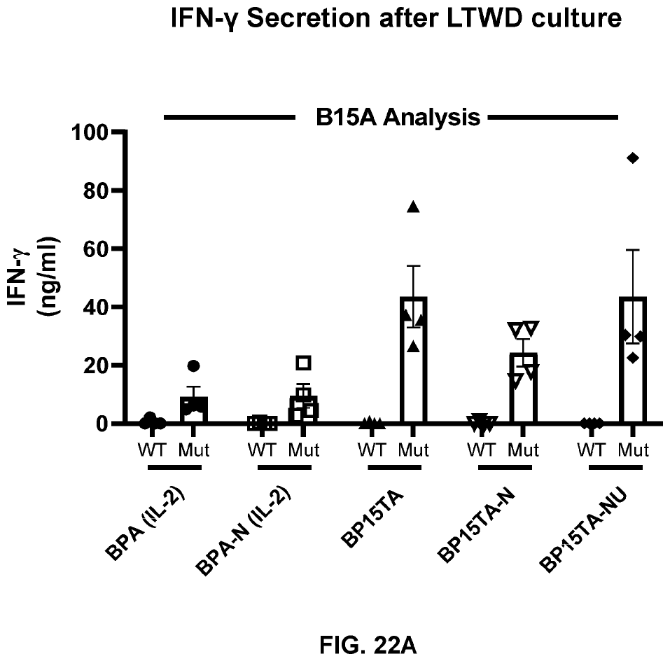

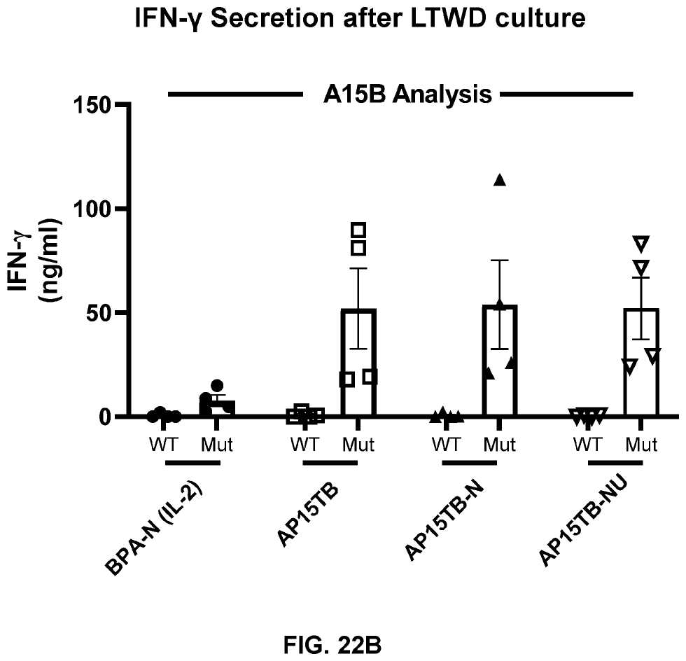

The activation of TCR-T cells after LTWD culture was assessed by 4-1BB induction and IFN-γ secretion after overnight co-culture with wild-type or mutant neoantigen (10 μg/mL) pulsed DCs (HLA matched). As described above, induction of 4-1BB on CD8+ T cells was detected with 4-1BB antibody (FIG. 21A-21B) and IFN-γ secretion was measured by ELISA (FIG. 22A-22B). These data demonstrate that mbIL15 TCR-T cells which survived LTWD culture retained their specific function and demonstrated more potent activation than T cells from control TCR only groups (IL-2 treated); however, the function of mbIL15 TCR-T cells generated with different versions of the polycistronic plasmids was not significantly different.

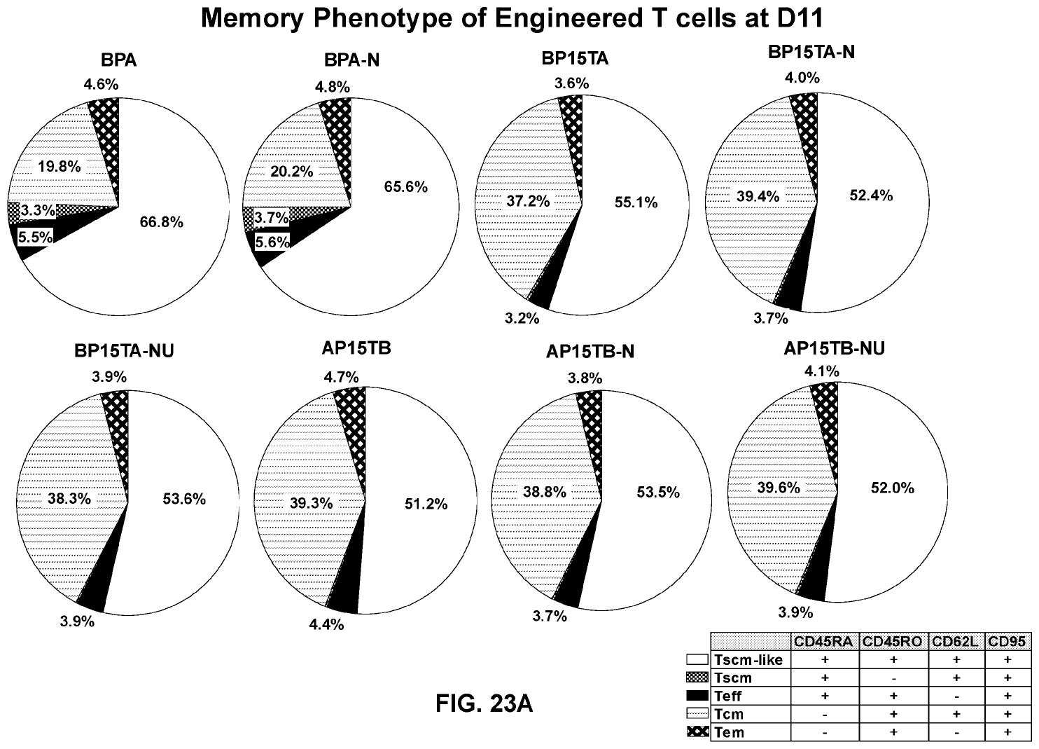

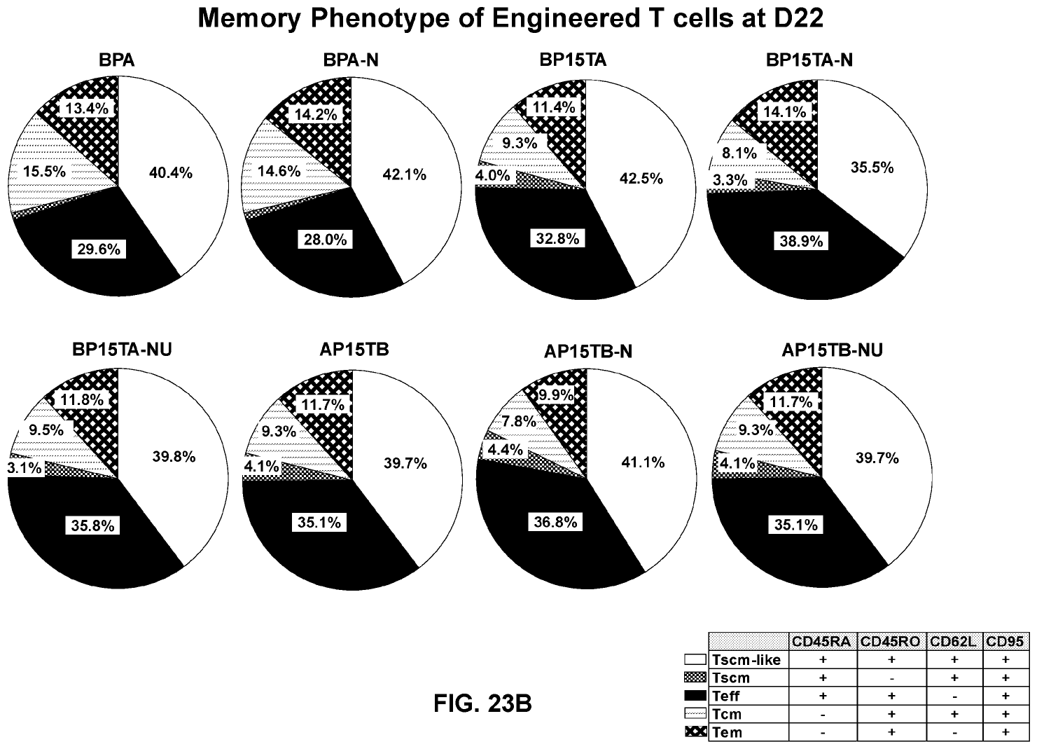

Memory phenotype of TCR-T cells electroporated with different polycistronic vectors was also assessed. T cell memory subsets are defined as: CD45RA+CD45RO+CD62L+CD95+=stem cell memory-like (Tscm-like); CD45RA+CD45RO−CD62L+CD95+=stem cell memory (Tscm); CD45RA-CD45RO+CD62L+CD95+=central memory (Tcm); CD45RA-CD45RO+CD62L-CD95+=effector memory (Tem). T cell effector (Teff) are defined as CD45RA+CD45RO+CD62L-CD95+. The pie charts in FIG. 23A-23C show the mean frequency of live CD3⁺ T cell memory and effector subsets at day 11 post-first phase expansion (FIG. 23A), day 22 post-second phase expansion (FIG. 23B), and after 4 weeks of LTWD culture (FIG. 23C) in cells transposed with the tested plasmids.

Memory phenotype data shows the kinetics of TCR-T memory and effector differentiation. At days 11 and 22 post-expansion, there is no difference between the different polycistronic TCR plasmids (FIG. 23A-23B). There were more Tscm and Teff cells and fewer Tcm cells in the TCR-T cells expressing mbIL15 relative to TCR-T cells without mbIL15. After 4 weeks of culture in absence or presence of IL-2, TCR-T cells predominantly differentiated into Teff cells (over 85%). TCR-T cells expressing mbIL15 cultured for 4 weeks in the absence of cytokines differentiated into 3 main subsets: Teff, Tscm-like and Tscm cells (FIG. 23C). These results suggest that mbIL15 is sufficient to support the Tscm phenotype.

Conclusions: The mbIL15 TCR-T cells generated from different versions of the polycistronic plasmids showed comparable features including TCR expression, memory phenotype, specificity, and IFN-γ secretion. This data supports that removal of cysteine-substitutions in the mouse constant domains used in the first-generation vectors and use of unified mouse constant regions will not produce any significant changes in the mbIL15 TCR-T cell product.

### 6.5 Example 5: Generation and Evaluation of T Cells Generated Using Various Tricistronic TCR/mbIL15 Vectors

This Example describes the evaluation of T cells expressing mbIL15 in combination with different TCRα/TCRβ chains generated using tricistronic vectors as described below. Similar to the vectors described in Example 4, the tricistronic expression cassettes used in this Example each include a transcriptional regulatory element operably linked to a polycistronic polynucleotide that encodes a TCR α chain (referred to herein as “TCRα” or “A”), a TCR β chain (referred to herein as “TCRβ” or “B”), and membrane-bound IL-15/IL-15Rα fusion protein (referred to herein as “mbIL15” or “15”), each separated by a furin recognition site and either a P2A element or a T2A element that mediates ribosome skipping to enable expression of separate polypeptide chains.

The nine TCRs used in this Example are each directed against a different target as shown in Table E10. The Vα amino acid sequences and Vβ amino acid sequences for each of the nine TCRs listed correspond to the sequences provided in Table 6. Each TCR α chain was generated by fusing the Vα sequence to a murine Cα region modified by substituting a leucine at amino acid position 112, an isoleucine at amino acid position 114, and a valine at amino acid position 115 (SEQ ID NO: 42). Each TCRβ chain was generated by fusing the Vβ sequence to a murine wild-type Co (SEQ ID NO: 52).

TABLE E10
TCR Targets.

| Target Protein | Mutation | HLA Type | TCR |
|---|---|---|---|
| TP53 | R175H | A*02:01 | TCR001 |
| | | DRB1*13:01 | TCR009 |
| | R248W | A*68:01 | TCR057 |
| | Y220C | DRB3*02:02 | TCR016 |
| KRAS | G12D | A*11:01 | TCR022 |
| | | C*08:02 | TCR064 |
| | G12V | A*11:01 | TCR075 |
| | | C*01:02 | TCR055 |
| EGFR | E746-A750del | DPA1*02:01/DPB1*01:01 | TCR077 |

For each of the TCRs above, three vectors were constructed and evaluated: 1) TCR only (BA); 2) A15B; and 3) B15A. The TCR only (BA) vectors contain a bicistronic expression cassette encoding TCR β chain and TCR α chain separated by a furin recognition site and a P2A element in the following orientation from 5′ to 3′: TCRβ-TCRα. The AP15 TB vectors contain a tricistronic expression cassette encoding TCR α chain, TCR β chain, and mbIL15 in the following orientation from 5′ to 3′: TCRα-mbIL15-TCRβ. The BP15TA vectors contain a tricistronic expression cassette encoding TCR α chain, TCR β chain, and mbIL15 in the following orientation from 5′ to 3′: TCRβ-mbIL15-TCRα.

TCR-T cells described in this Example were generated similarly to those described in Examples 2-4 except as indicated below. Where it is indicated that cells were transposed, cells were co-electroporated with the indicated plasmid as well as plasmid TA or similar Transposase expression plasmid unless otherwise stated.

Briefly, cryopreserved PBMCs were thawed, resuspended in 50:50 media and placed in a 37° C./5% CO₂ incubator before electroporation.

Test articles as listed in Table E11 were then prepared.

TABLE E11
PBMC test articles.

| Group | TCR | Name | Description |
|---|---|---|---|
| 3.1 | None | NT | Non-transposed PBMCs |
| 3.2 | TCR001 | BPA-N¹ | PBMCs transposed with TCR001 BPA-N |
| 3.3 | TCR001 | AP15TB-NU² | PBMCs transposed with TCR001 AP15TB-NU |
| 3.4 | TCR001 | BP15TA-NU³ | PBMCs transposed with TCR001 BP15TA-NU |
| 3.5 | TCR057 | BPA-N | PBMCs transposed with TCR057 BPA-N |
| 3.6 | TCR057 | AP15TB-NU | PBMCs transposed with TCR057 AP15TB-NU |
| 3.7 | TCR057 | BP15TA-NU | PBMCs transposed with TCR057 BP15TA-NU |
| 3.8 | TCR009 | BPA-N | PBMCs transposed with TCR009 BPA-N |
| 3.9 | TCR009 | AP15TB-NU | PBMCs transposed with TCR009 AP15TB-NU |
| 3.10 | TCR009 | BP15TA-NU | PBMCs transposed with TCR009 BP15TA-NU |
| 3.11 | TCR016 | BPA-N | PBMCs transposed with TCR016 BPA-N |
| 3.12 | TCR016 | AP15TB-NU | PBMCs transposed with TCR016 AP15TB-NU |
| 3.13 | TCR016 | BP15TA-NU | PBMCs transposed with TCR016 BP15TA-NU |
| 3.14 | None | NT | Non-transposed PBMCs |
| 3.15 | TCR022 | BPA-N | PBMCs transposed with TCR022 BPA-N |
| 3.16 | TCR022 | AP15TB-NU | PBMCs transposed with TCR022 AP15TB-NU |
| 3.17 | TCR022 | BP15TA-NU | PBMCs transposed with TCR022 BP15TA-NU |
| 3.18 | TCR075 | BPA-N | PBMCs transposed with TCR075 BPA-N |
| 3.19 | TCR075 | AP15TB-NU | PBMCs transposed with TCR075 AP15TB-NU |
| 3.20 | TCR075 | BP15TA-NU | PBMCs transposed with TCR075 BP15TA-NU |
| 3.21 | TCR055 | BPA-N | PBMCs transposed with TCR055 BPA-N |
| 3.22 | TCR055 | AP15TB-NU | PBMCs transposed with TCR055 AP15TB-NU |
| 3.23 | TCR055 | BP15TA-NU | PBMCs transposed with TCR055 BP15TA-NU |
| 3.24 | TCR064 | BPA-N | PBMCs transposed with TCR064 BPA-N |
| 3.25 | TCR064 | AP15TB-NU | PBMCs transposed with TCR064 AP15TB-NU |
| 3.26 | TCR064 | BP15TA-NU | PBMCs transposed with TCR064 BP15TA-NU |
| 3.27 | None | NT | Non-transposed PBMCs |
| 3.28 | TCR077 | BPA-N | PBMCs transposed with TCR077 BPA-N |
| 3.29 | TCR077 | AP15TB-NU | PBMCs transposed with TCR077 AP15TB-NU |
| 3.30 | TCR077 | BP15TA-NU | PBMCs transposed with TCR077 BP15TA-NU |

¹Generated using the same plasmid as BPA-N group in Example 4.
²Generated using the same plasmid as AP15TB-NU group in Example 4.
³Generated using the same plasmid as BP15TA-NU group in Example 4.

Test articles were prepared in three batches (Batch 1=Groups 3.1-3.13; Batch 2=Groups 3.14-3.26; Batch 3=Groups 3.27-3.30) as follows:

Groups 3.1, 3.14, & 3.27: Cells were harvested, spun down, resuspended in recovery media (50:50 media containing IL-7+IL-15+n-acetylcysteine (NAC)), and incubated in a 37° C./5% CO₂ incubator overnight.

Groups 3.2-3.13, 3.15-3.26, & 3.28-3.30: Cells were harvested, spun down, resuspended in electroporation buffer together with the plasmids listed in Table E10, and electroporated. Following electroporation, cell suspensions were collected, transferred to recovery media (50:50 media containing IL-7+IL-15+NAC), and incubated in a 37° C./5% CO₂ incubator overnight.

Within 24 hours post-electroporation (Day 1), live cells were transferred to G-REX® culture plates (Wilson Wolf Manufacturing) and incubated with a first expansion media (50:50 media containing IL-21+IL-7+IL-12+ T Cell TransAct™). Cells were fed regularly with cytokines. After 11 days of first phase expansion, TCR+ cells were isolated with mTCR antibody. The isolated TCR+ T cells were transferred to G-REX® culture plates (Wilson Wolf Manufacturing) and incubated with a second expansion media (50:50 media containing 3000 U/ml of IL-2+ T Cell TransAct™). During this second expansion phase, cells were fed regularly with cytokines. After 11 or 16 days of second phase expansion, cells were harvested, and the various assays described below were performed.

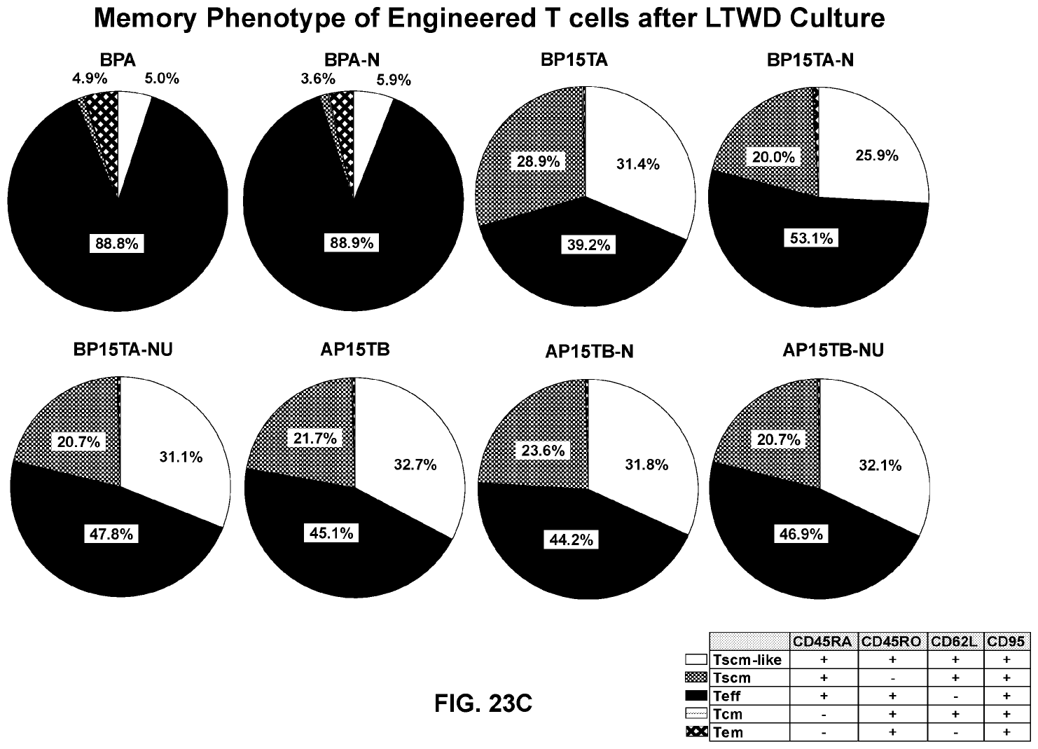

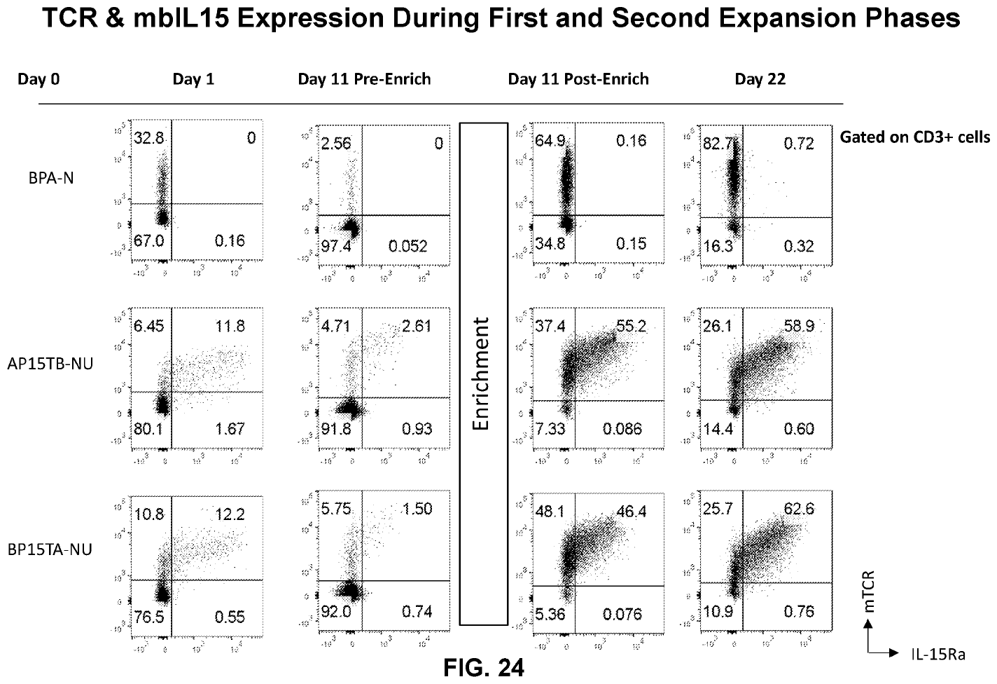

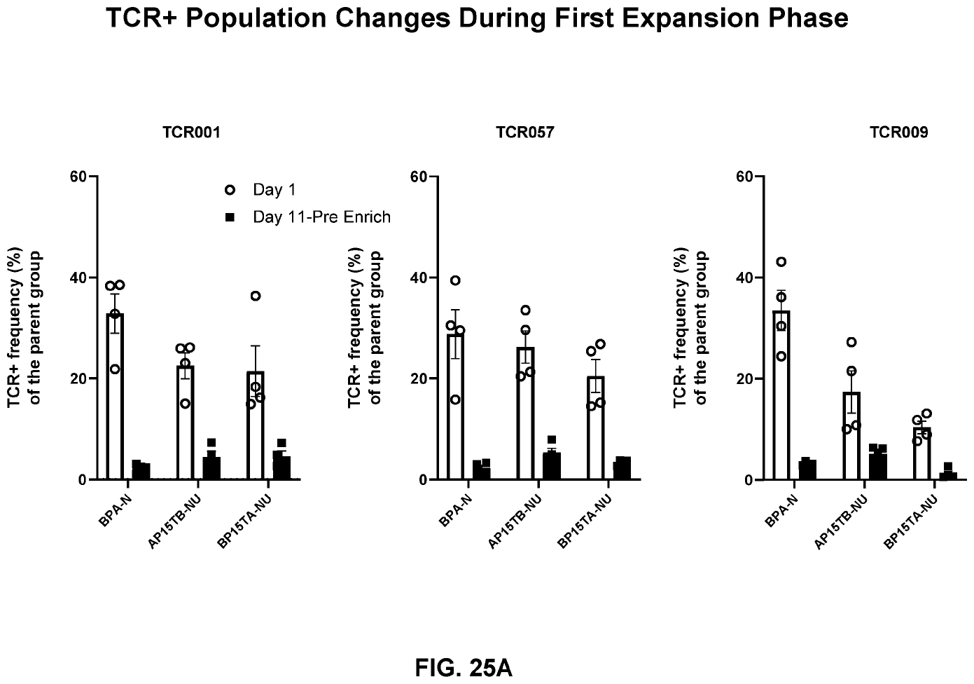

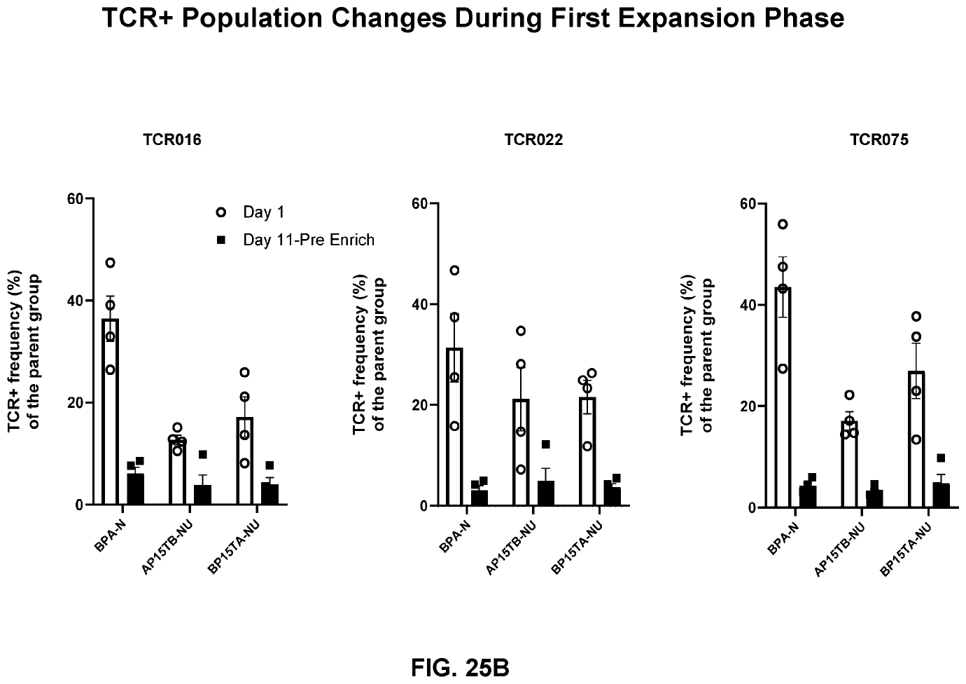

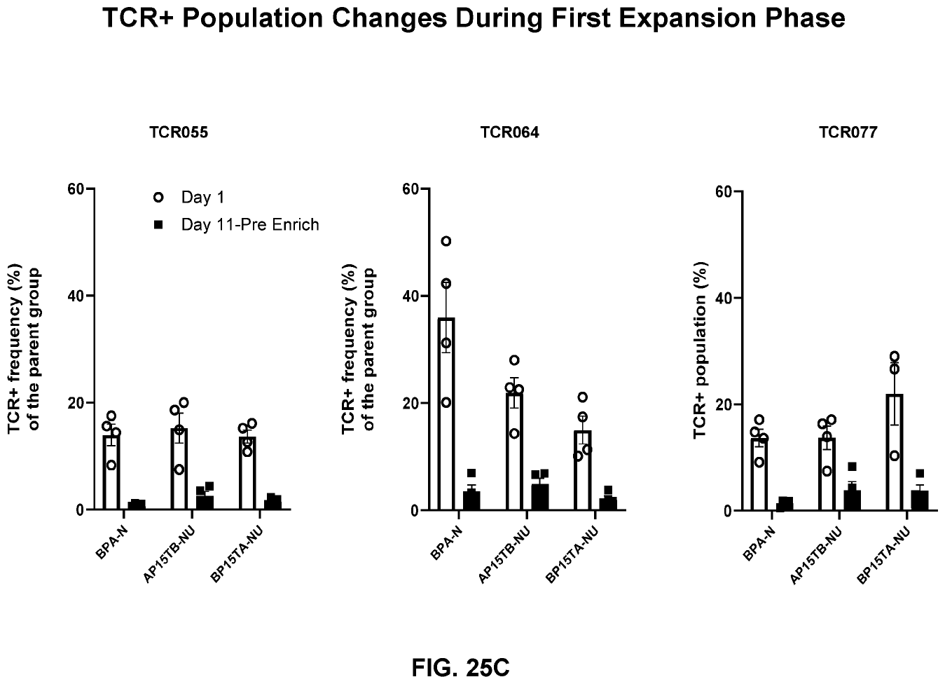

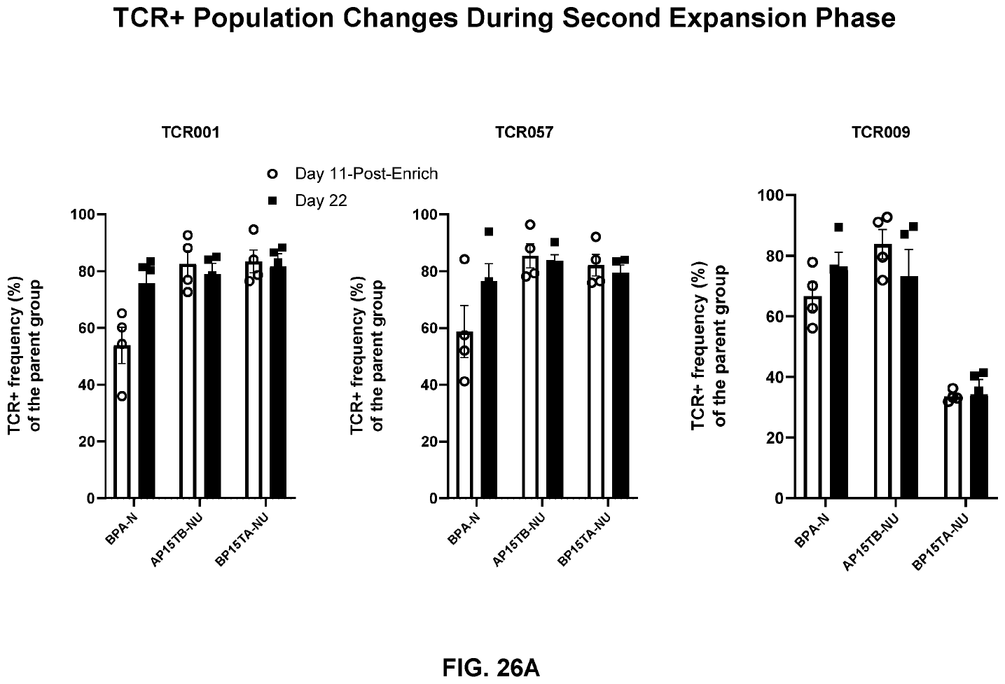

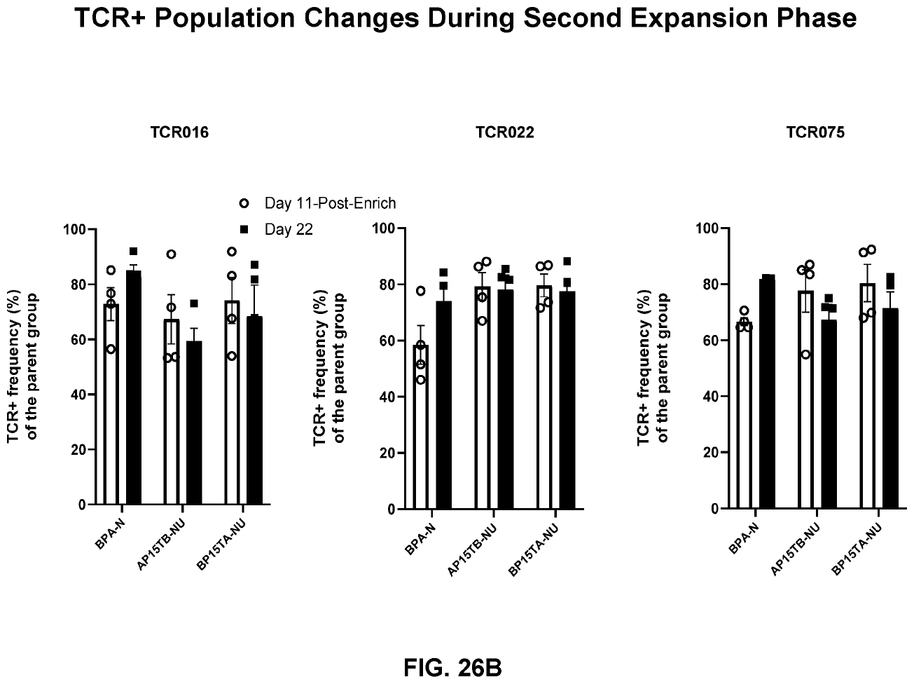

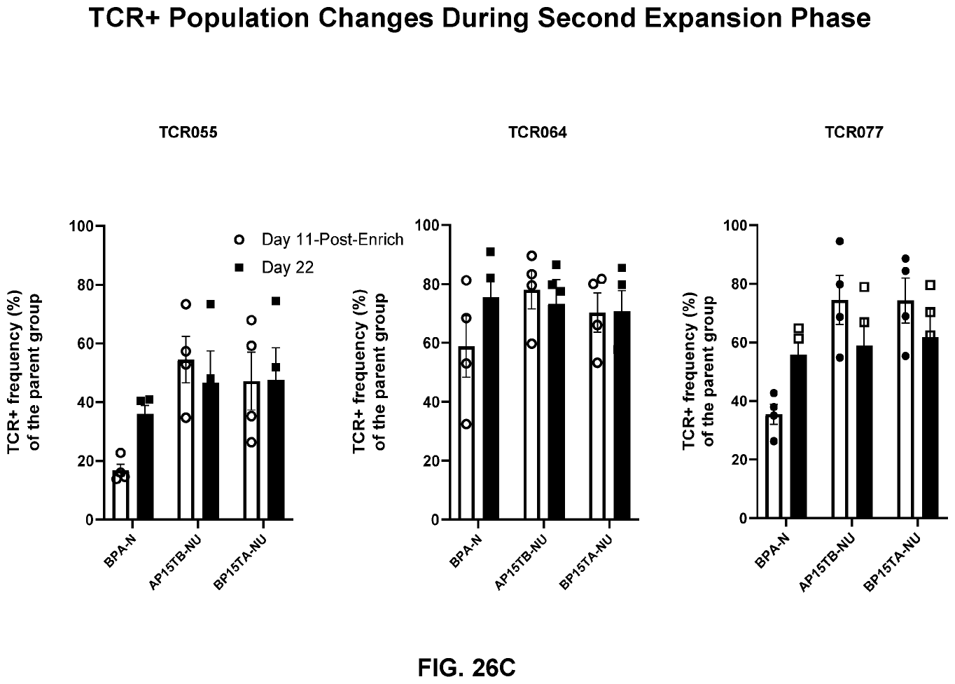

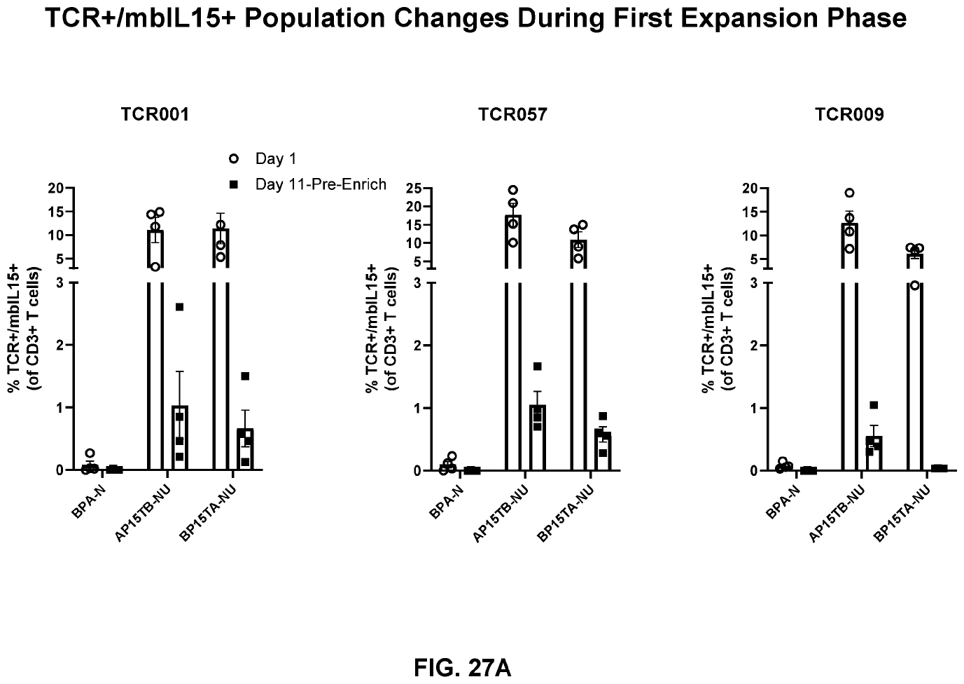

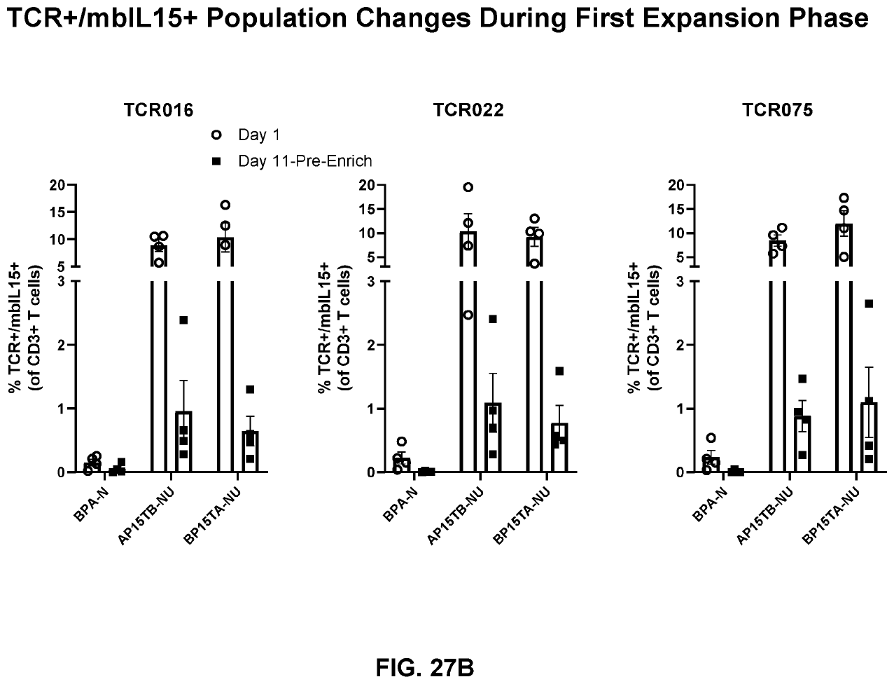

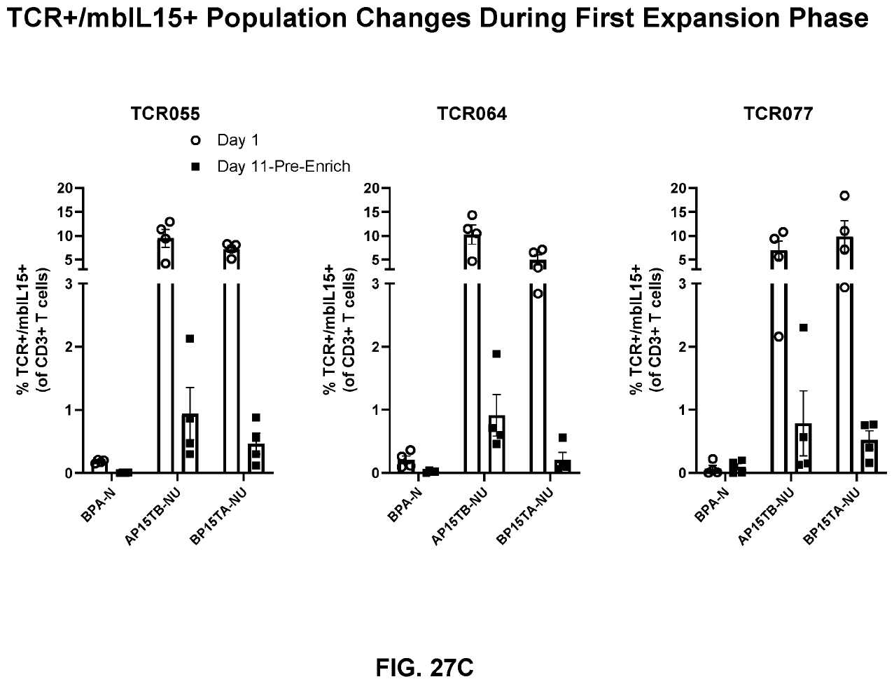

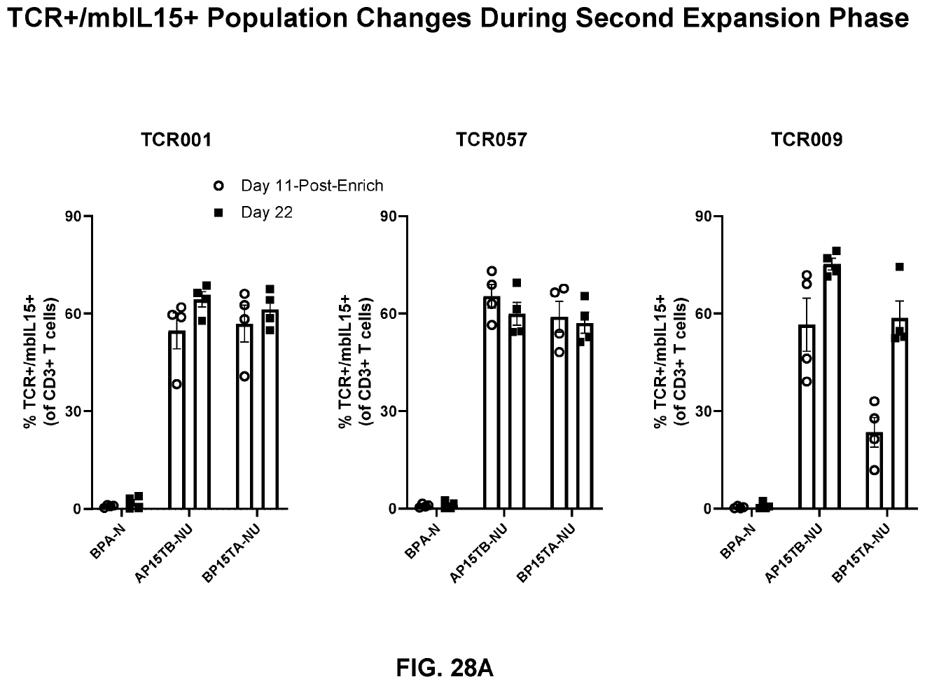

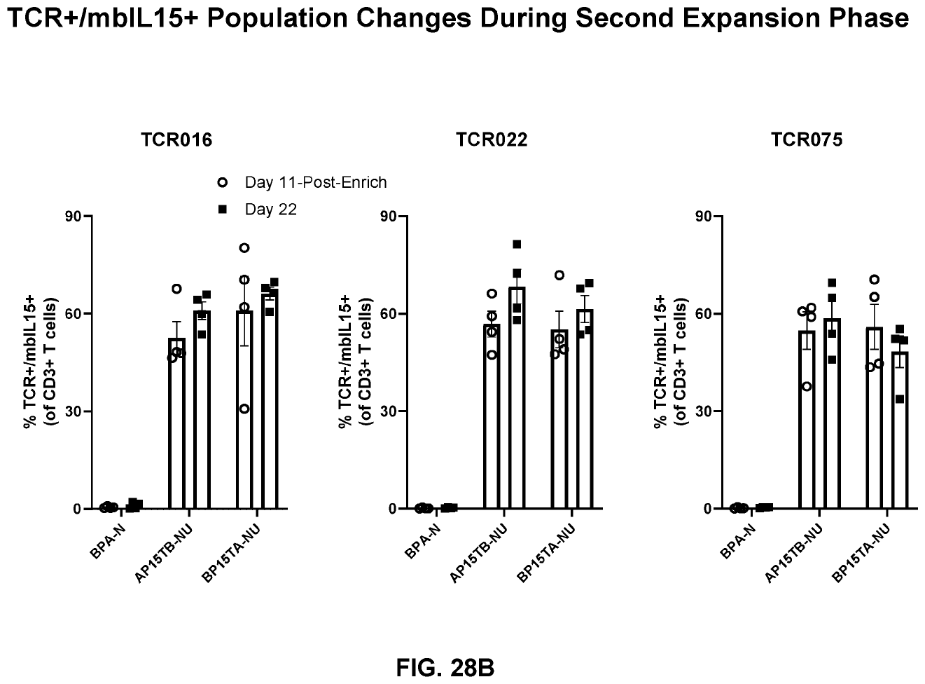

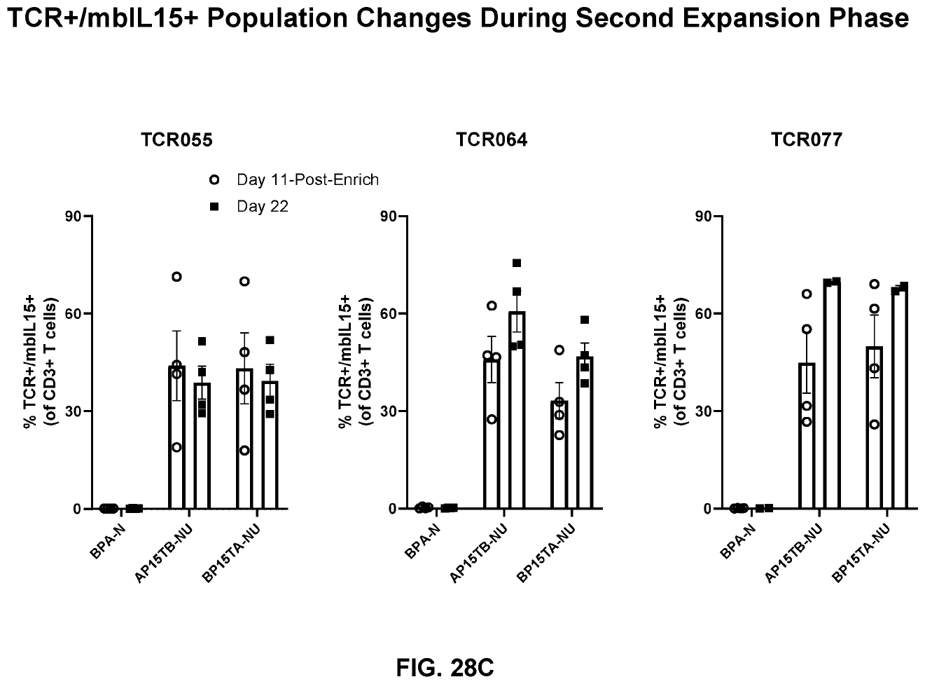

Transgene expression was assessed for T cells electroporated with different polycistronic plasmids. On Day 1 (post-electroporation), Day 11 (post-1′ phase expansion) and Day 22 (post-2^{nd} phase expansion), cells were harvested and the expression of mTCR and mbIL15 was detected on CD3+ gated population with mouse TCR beta antibody and IL-15Rα antibody. The results are shown in FIGS. 24-28.

Fold expansion of total cell count and mTCR+ cell count was assessed for T cells electroporated with different polycistronic plasmids. Fold expansion value was calculated as: Cell number on Day 11/Cell number on Day 1 and Cell number on Day 22/Cell number on Day 11. mTCR+ cell number was calculated as: Total cell number X CD3 population (%) X mTCR population (%). The results are shown in Table E12.

TABLE E12
Fold expansion of total cells and mTCR+ cell count during first and second
expansion phases.

| | | Fold Expansion (FE) | | | | | | | |
|---|---|---|---|---|---|---|---|---|---|
| | | Total Cell | | | | mTCR+ Cell | | | |
| | | First expansion | | Second expansion | | First expansion | | Second expansion | |
| Group | | Mean | SEM | Mean | SEM | Mean | SEM | Mean | SEM |
| 1 | TCR001-BPA-N | 83.86 | 19.60 | 121.97 | 11.81 | 6.11 | 2.09 | 171.56 | 9.57 |
| | TCR001-AP15TB-NU | 66.89 | 13.09 | 107.01 | 20.38 | 18.38 | 9.60 | 101.00 | 17.14 |
| | TCR001-BP15TA-NU | 62.93 | 17.70 | 108.50 | 20.15 | 19.04 | 7.72 | 104.08 | 16.79 |
| 2 | TCR057-BPA-N | 98.21 | 36.03 | 110.40 | 7.05 | 8.16 | 4.73 | 146.17 | 13.45 |
| | TCR057-AP15TB-NU | 58.90 | 16.21 | 107.38 | 16.76 | 17.34 | 9.77 | 104.42 | 16.05 |
| | TCR057-BP15TA-NU | 58.78 | 16.21 | 108.69 | 3.91 | 14.19 | 6.44 | 104.04 | 3.64 |
| 3 | TCR009-BPA-N | 104.97 | 30.03 | 142.34 | 25.63 | 11.62 | 4.92 | 154.58 | 28.38 |
| | TCR009-AP15TB-NU | 51.68 | 13.48 | 95.38 | 13.31 | 24.76 | 12.75 | 83.83 | 16.19 |
| | TCR009-BP15TA-NU | 49.99 | 4.70 | 75.43 | 10.92 | 7.03 | 3.49 | 77.03 | 17.45 |
| 4 | TCR016-BPA-N | 87.13 | 9.63 | 127.79 | 25.67 | 17.38 | 5.29 | 142.91 | 26.78 |
| | TCR016-AP15TB-NU | 61.46 | 12.58 | 95.42 | 16.34 | 26.02 | 18.67 | 82.78 | 10.34 |
| | TCR016-BP15TA-NU | 66.33 | 15.42 | 93.38 | 14.09 | 23.39 | 15.51 | 83.30 | 14.44 |
| 5 | TCR022-BPA-N | 85.99 | 6.51 | 169.56 | 16.12 | 8.39 | 1.15 | 202.20 | 37.63 |
| | TCR022-AP15TB-NU | 59.11 | 5.90 | 95.36 | 12.47 | 12.21 | 1.59 | 102.12 | 22.10 |
| | TCR022-BP15TA-NU | 63.98 | 5.28 | 120.10 | 17.30 | 11.70 | 1.79 | 161.60 | 69.66 |
| 6 | TCR075-BPA-N | 89.45 | 6.14 | 182.86 | 15.27 | 9.12 | 0.75 | 207.74 | 21.54 |
| | TCR075-AP15TB-NU | 63.30 | 7.06 | 106.07 | 14.15 | 12.71 | 2.11 | 102.37 | 32.40 |
| | TCR075-BP15TA-NU | 59.06 | 5.91 | 107.40 | 20.65 | 10.57 | 2.47 | 114.25 | 43.15 |
| 7 | TCR055-BPA-N | 78.21 | 3.52 | 165.43 | 25.40 | 6.05 | 1.34 | 198.11 | 98.27 |
| | TCR055-AP15TB-NU | 58.84 | 1.25 | 95.98 | 18.09 | 10.28 | 1.79 | 67.59 | 20.05 |
| | TCR055-BP15TA-NU | 59.15 | 6.69 | 87.57 | 13.96 | 8.76 | 2.03 | 78.66 | 25.73 |
| 8 | TCR064-BPA-N | 86.10 | 10.54 | 177.48 | 15.01 | 8.47 | 1.48 | 212.18 | 55.16 |
| | TCR064-AP15TB-NU | 55.46 | 5.95 | 120.98 | 12.99 | 12.61 | 1.78 | 162.44 | 67.74 |
| | TCR064-BP15TA-NU | 59.66 | 4.38 | 110.54 | 16.75 | 9.78 | 1.98 | 137.63 | 59.08 |
| 9 | TCR077-BPA-N | 91.68 | 6.43 | 217.14 | 17.84 | 12.72 | 5.26 | 332.66 | 29.27 |
| | TCR077-AP15TB-NU | 57.10 | 5.54 | 97.22 | 13.45 | 16.66 | 7.28 | 75.79 | 4.98 |
| | TCR077-BP15TA-NU | 63.14 | 3.27 | 94.38 | 19.73 | 13.41 | 3.60 | 75.86 | 14.16 |

Cells generated using the polycistronic plasmids containing different TCR sequences were not phenotypically different from each other as demonstrated by transgene expression and cell growth data.

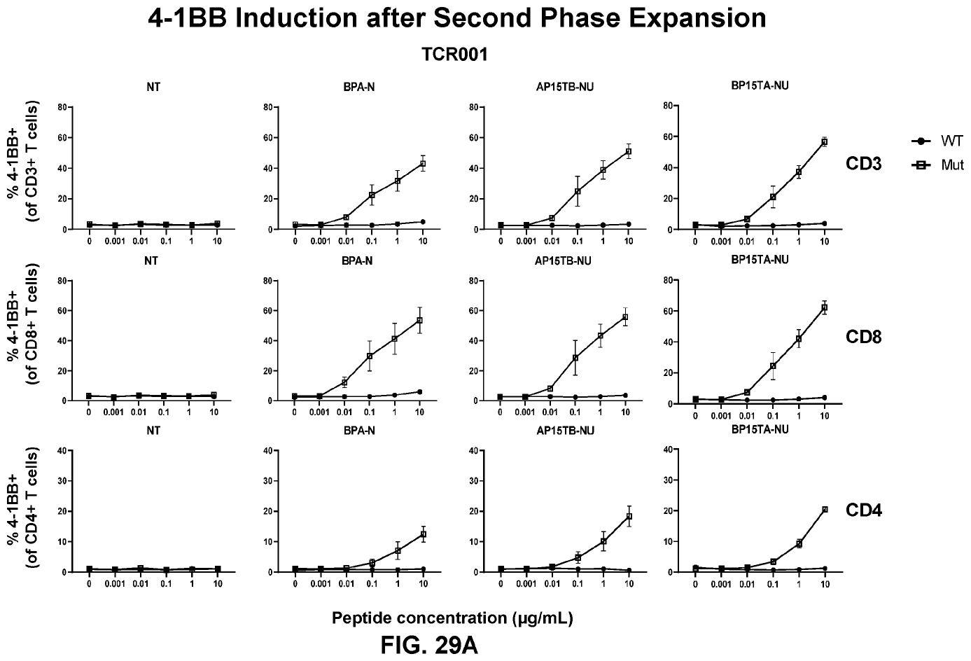

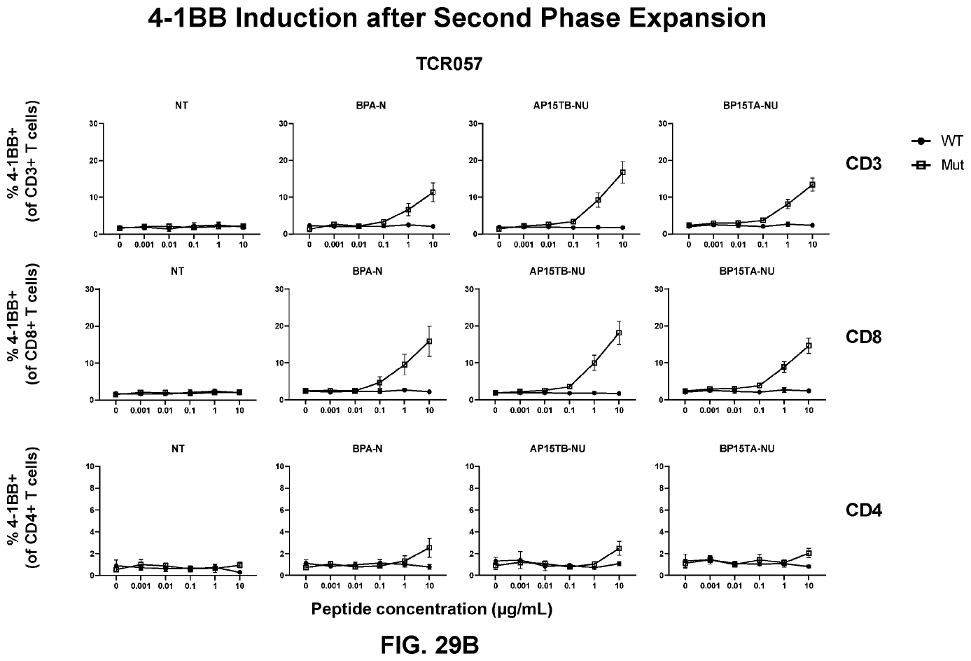

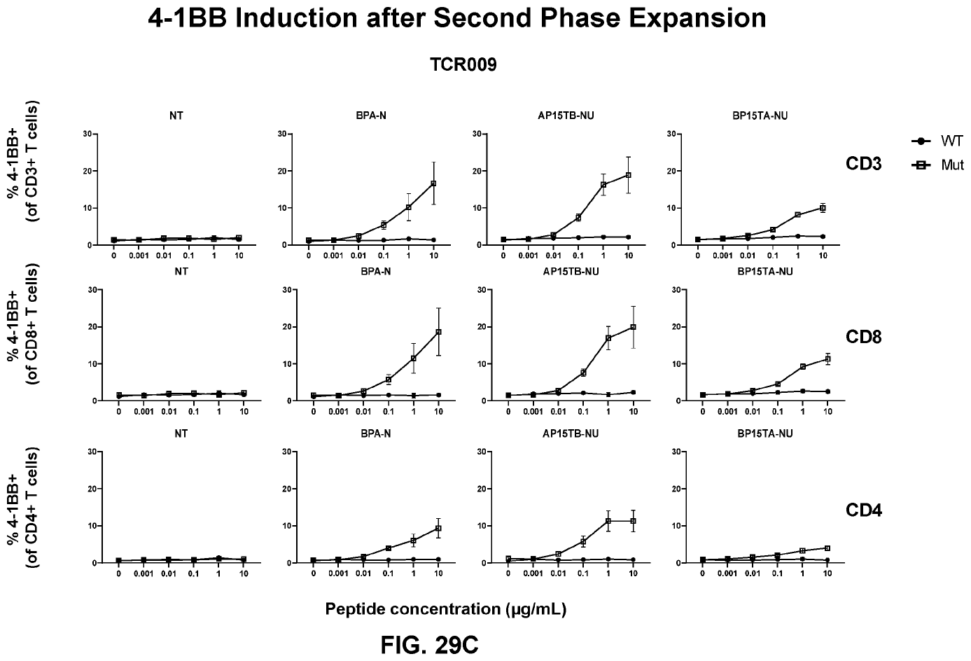

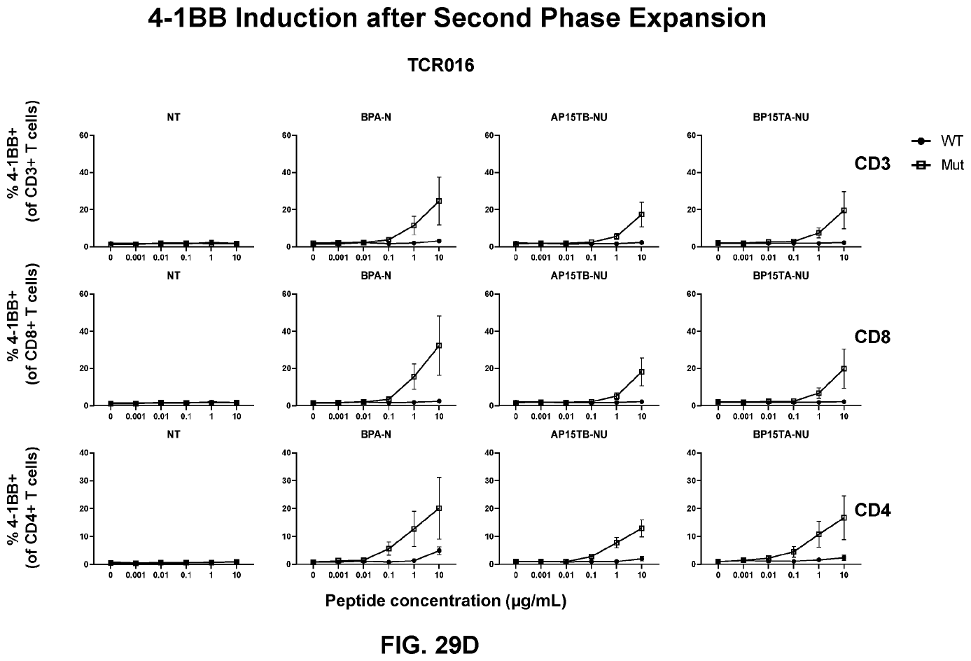

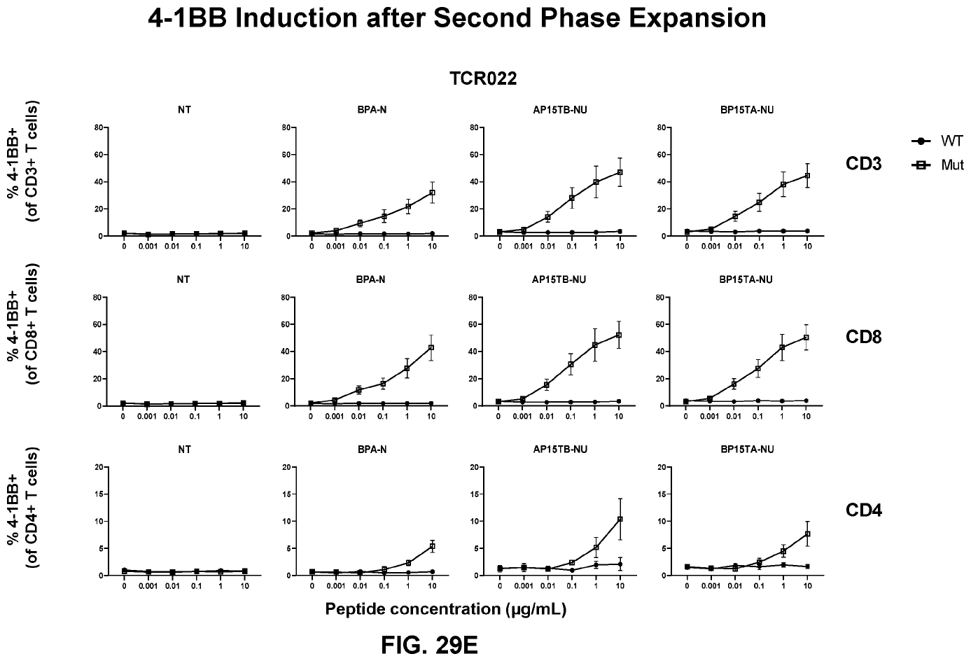

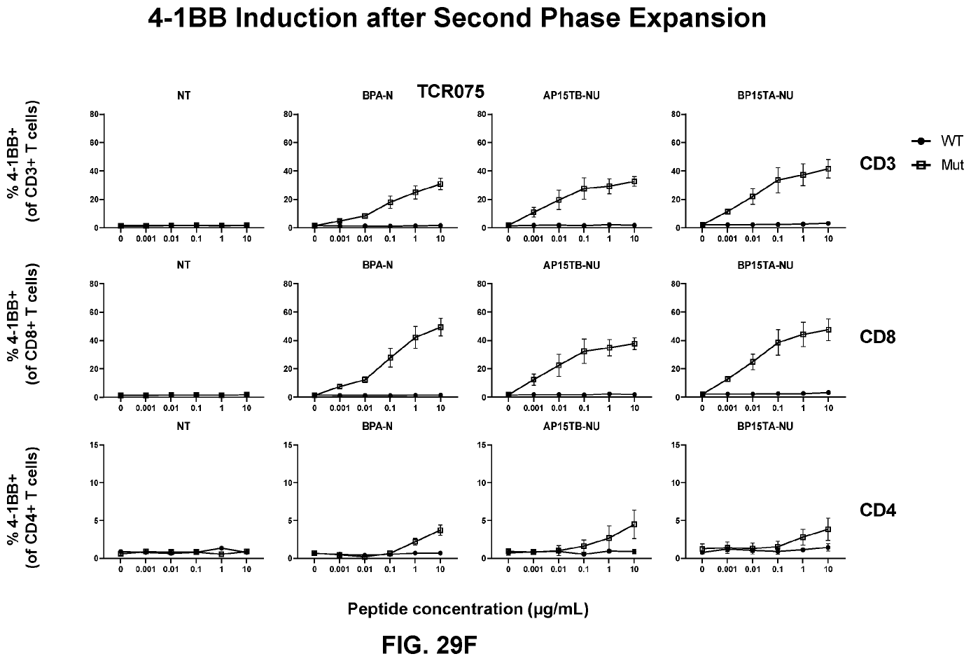

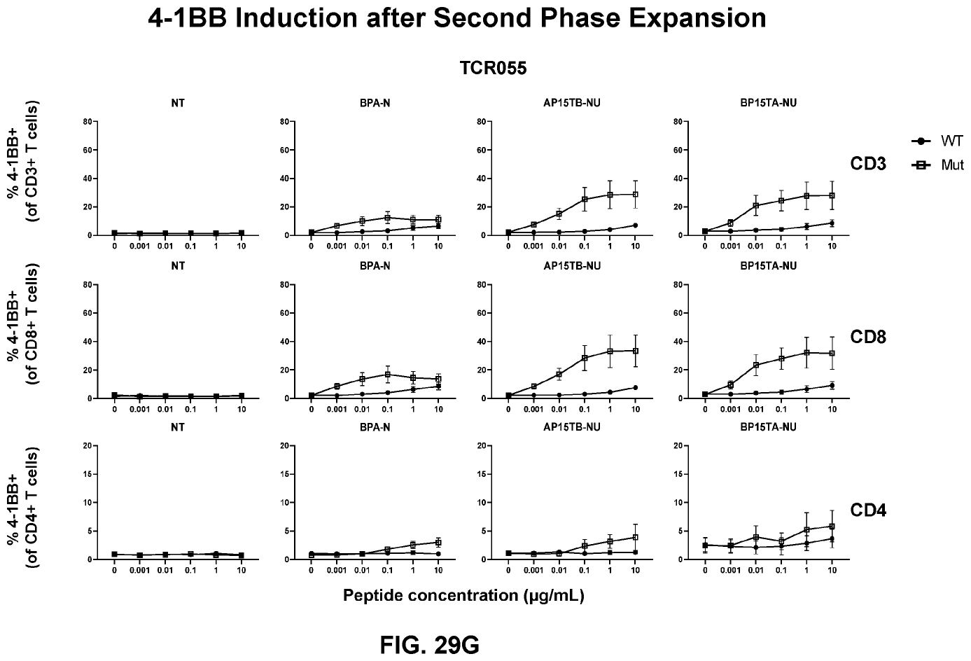

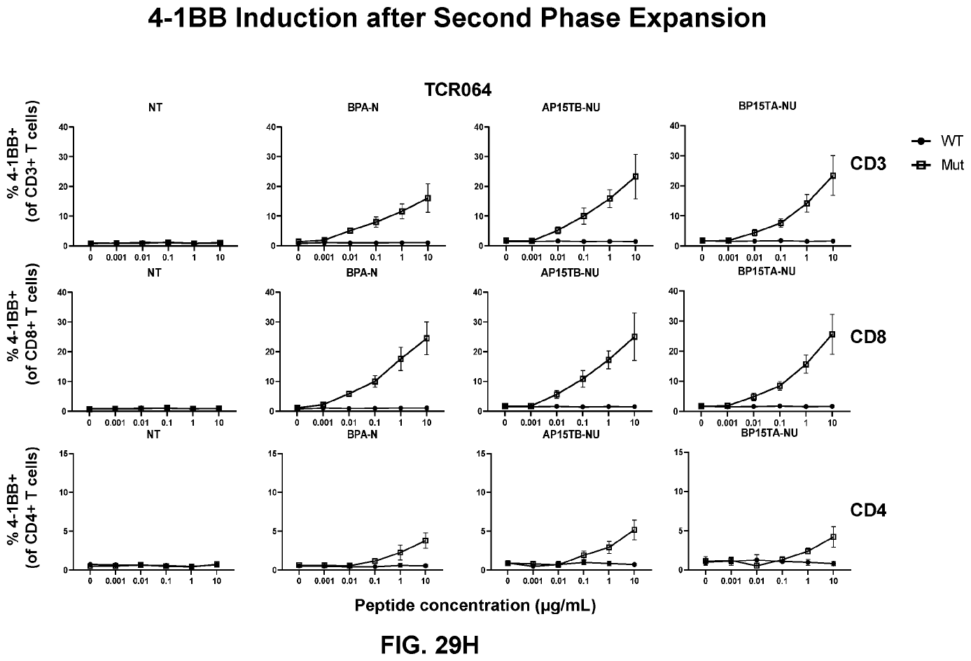

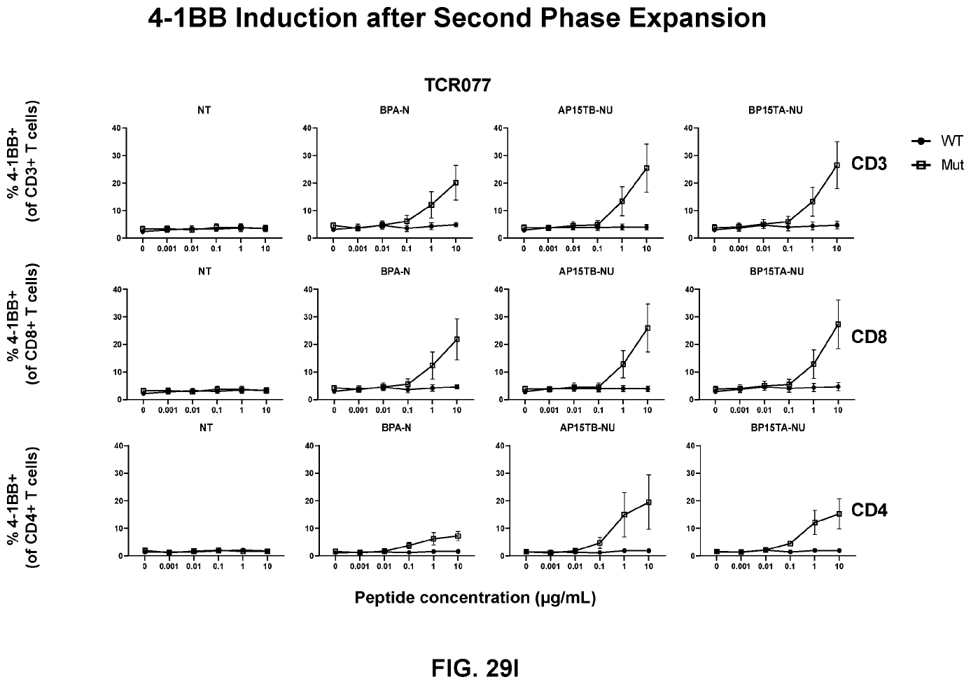

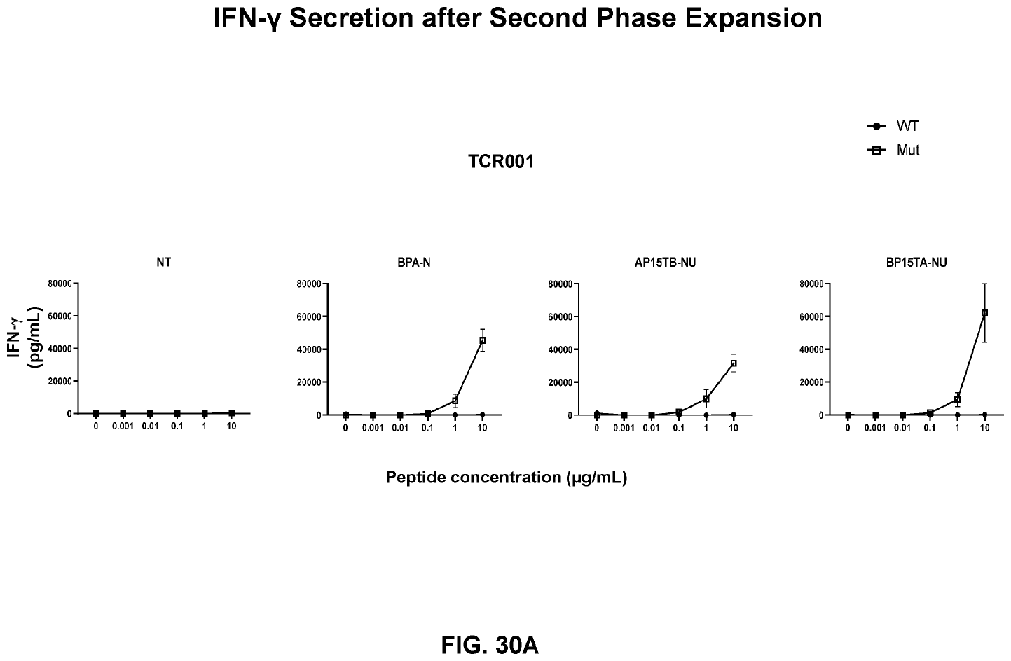

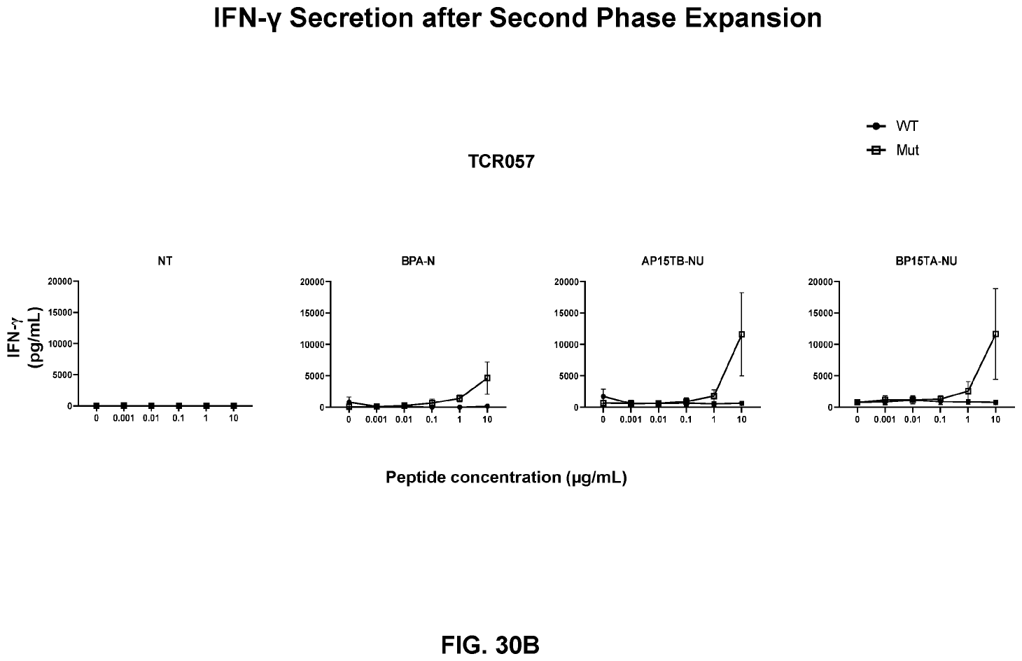

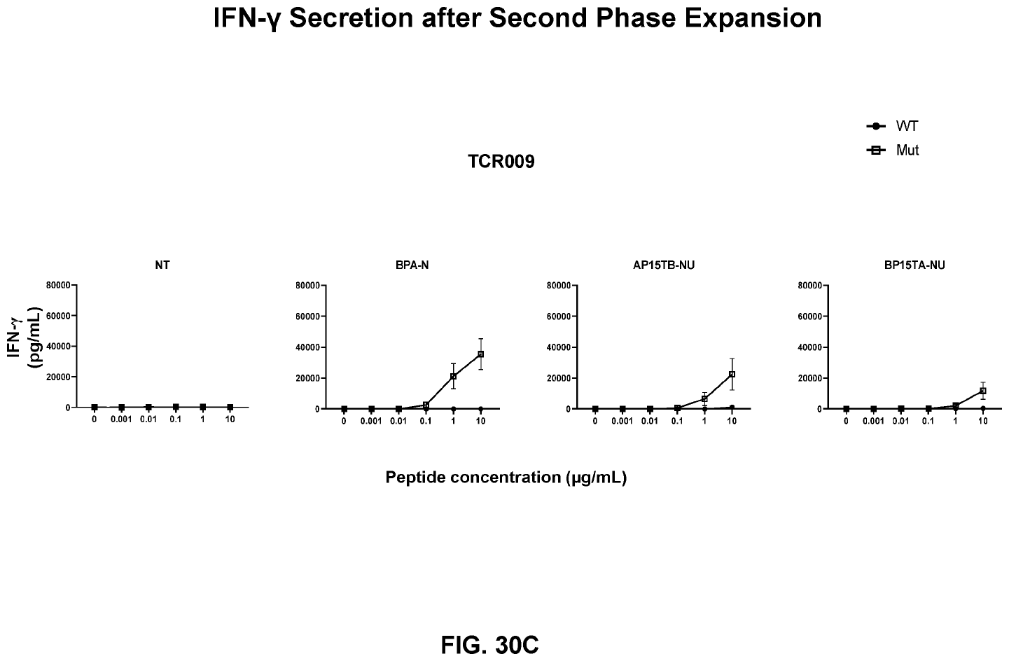

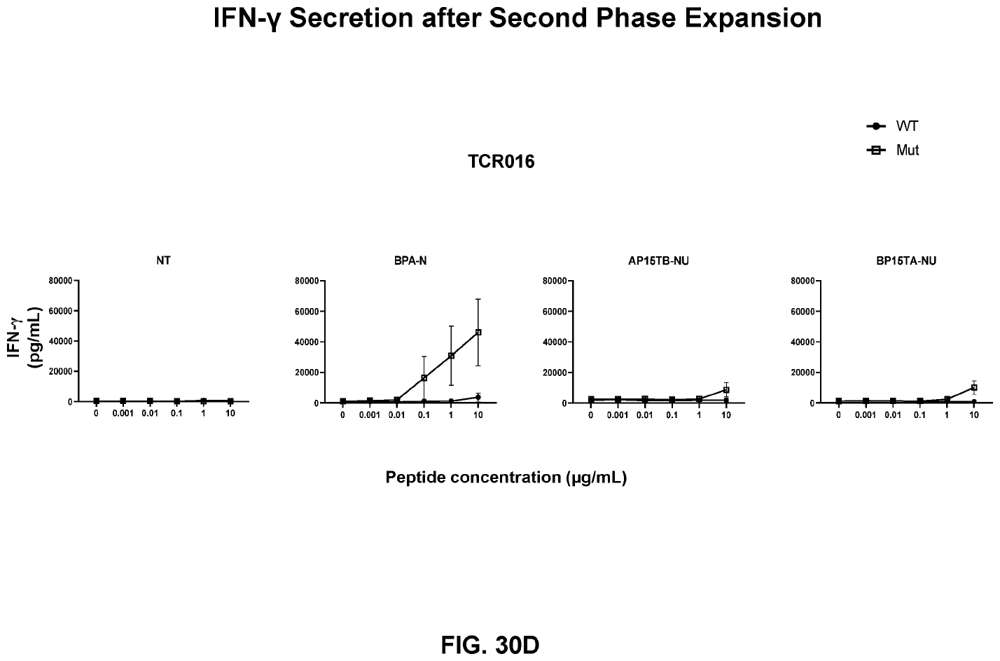

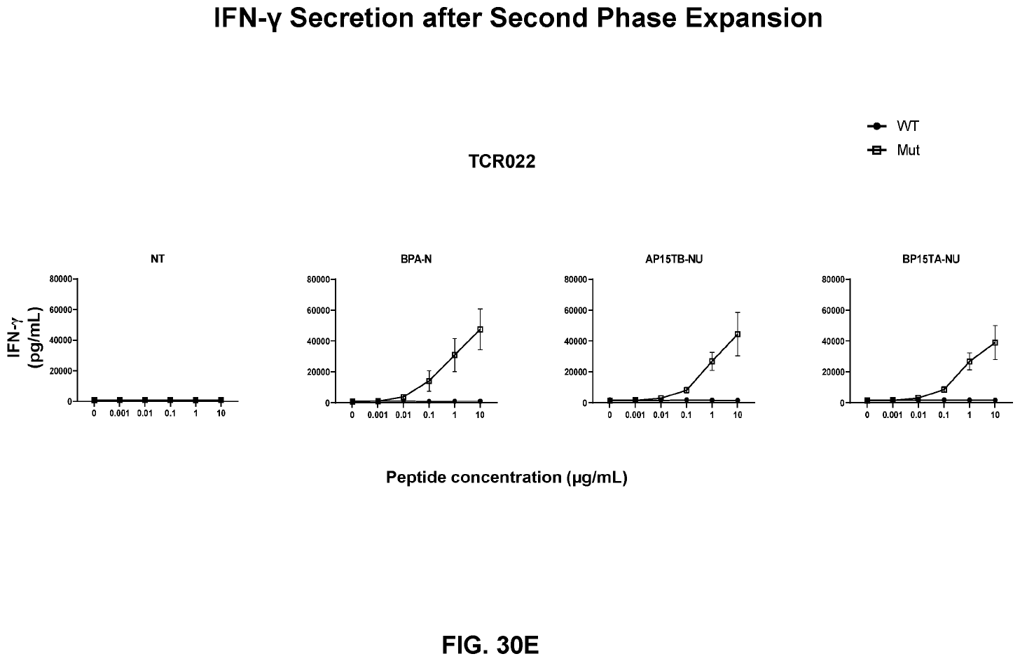

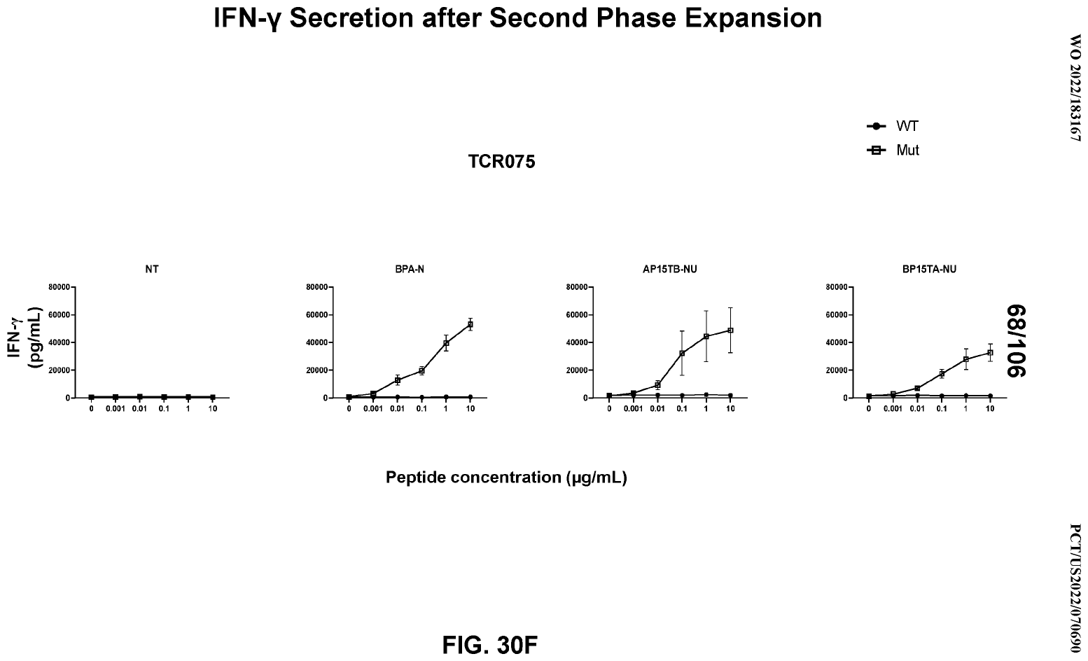

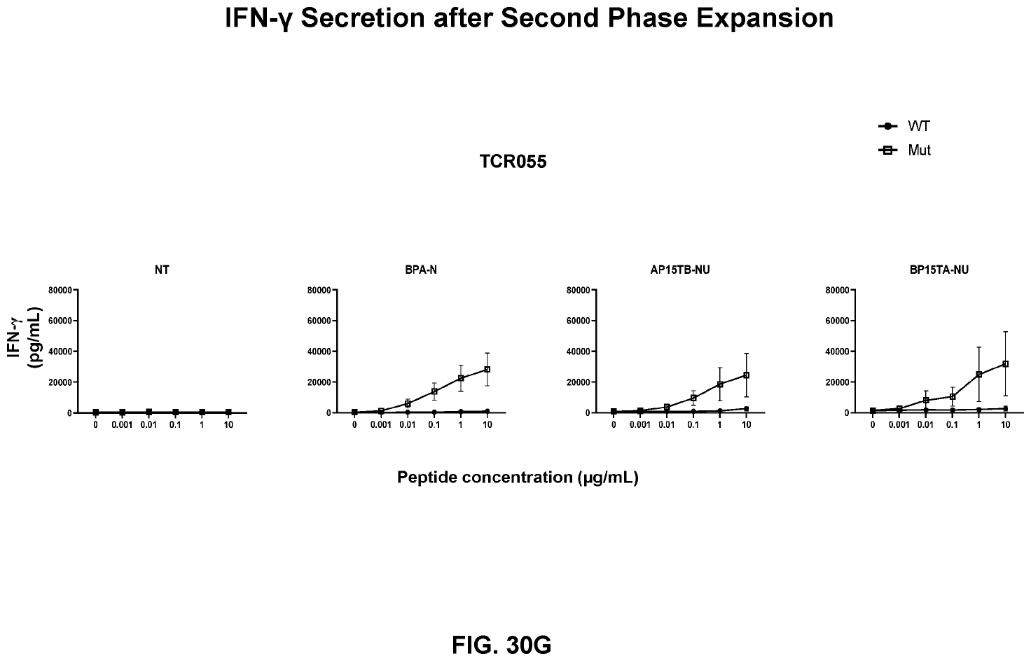

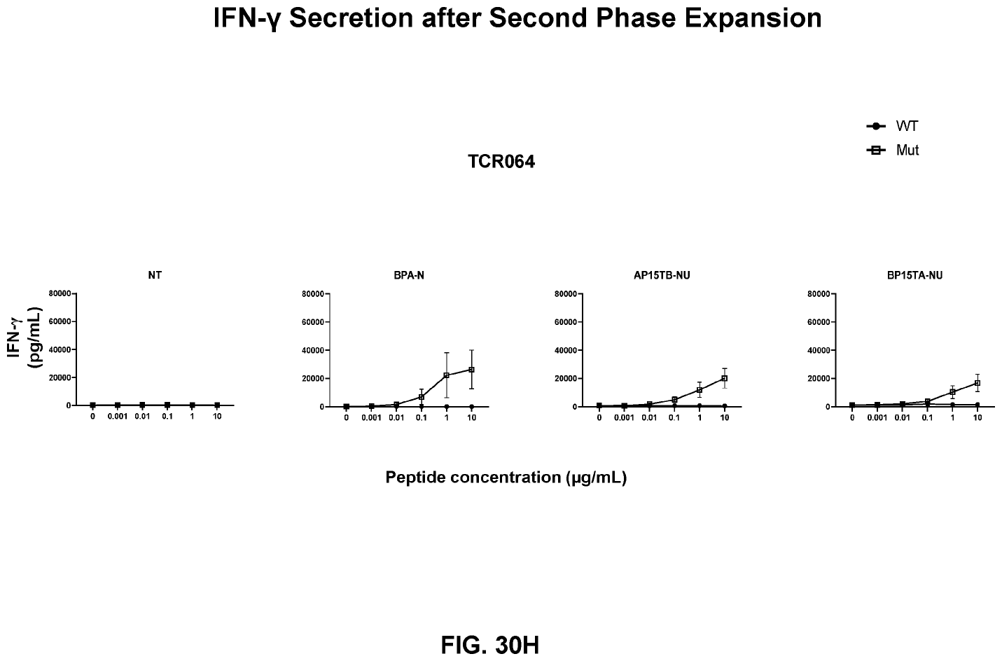

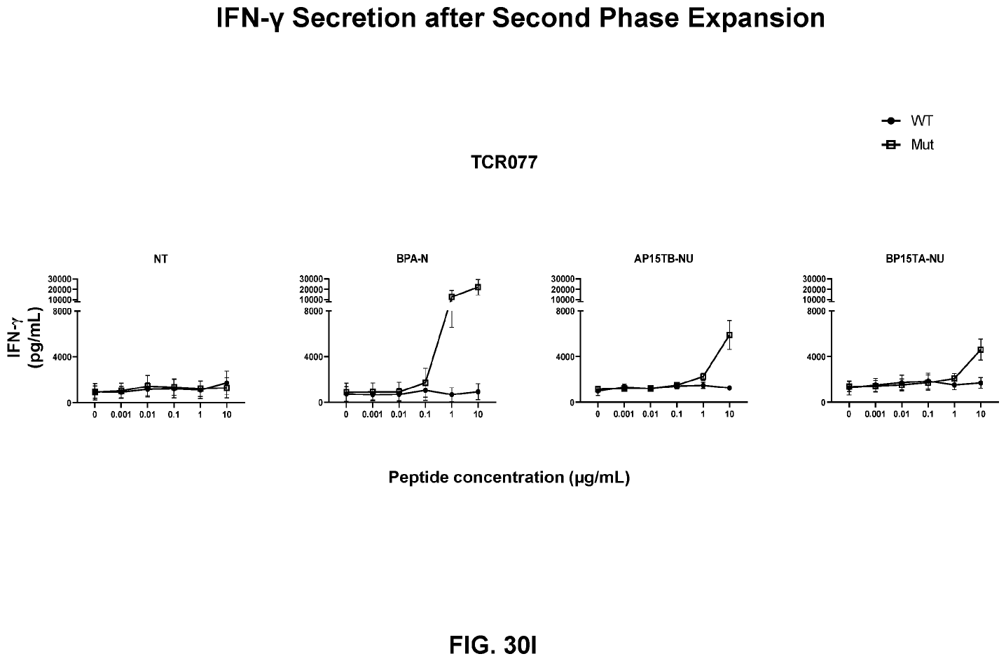

To assess activation of the generated TCR-T cells, overnight co-culture of the generated TCR-T cells with wild-type or mutant neoantigen pulsed DCs (HLA matched) was performed after 16 days second phase expansion and 4-1BB induction and IFN-γ secretion were measured. Induction of 4-1BB on CD3+CD8+ cells was detected with 4-1BB antibody. Secretion of IFN-γ measured with the ELISA antibody pair. The 4-1BB induction results are shown in FIG. 29A-29I and IFN-γ secretion results are shown in FIG. 30A-30I. The results demonstrate that when challenged with their cognate neoantigen, mbIL-15 TCR-T cells were highly avid and specific to the target neoantigens as measured by upregulation of 4-1BB co-stimulatory receptor and secretion of IFN-γ with negligible recognition of wild type sequences.

All data from electroporation to the second expansion phase of TCR vetting demonstrated that tricistronic system, expressing TCRα, TCRβ and mbIL15 with one plasmid successfully generated mbIL15 TCR-T cells and the features of the generated mbIL15 TCR-T cells are comparable to TCR-T cells in terms of transgene expression, cell growth, and functional specificity (4-1BB induction and IFN-γ secretion).

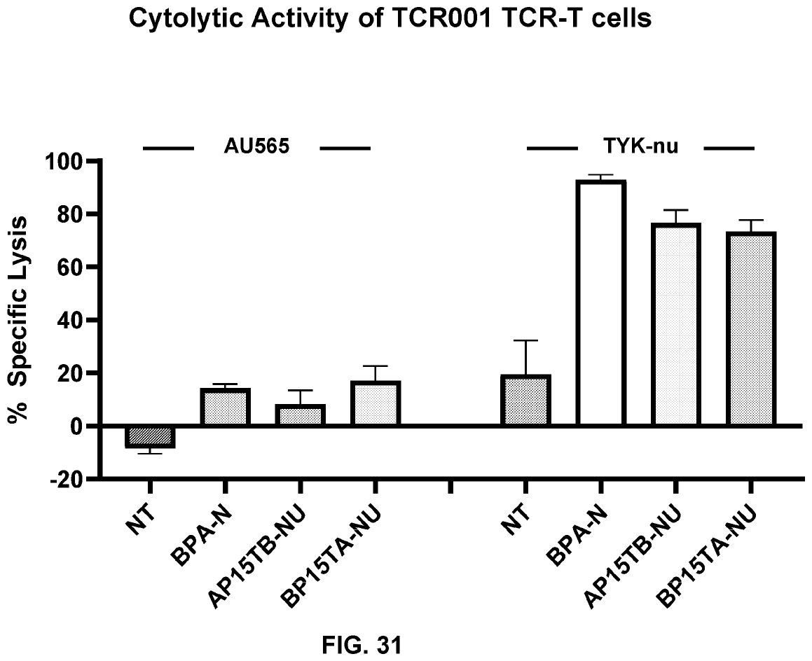

Cytolytic activity of TCR-T cells was assessed for T cells electroporated with polycistronic plasmids encoding TCR001+/−mbIL15 generated as described above (overnight recovery+11 days first phase expansion+11 days second phase expansion) and then harvested and frozen on Day 22. On experimental day, frozen Day 22 TCR-T cells were thawed and recovered for 3 days in media containing 3000 U/ml of IL-2. Then, the recovered TCR-T cells were incubated with AU565 (Mut+HLAneg) or Tyk-nu (Mut+HLA+) cells. After overnight incubation, the remaining T cells were extensively washed, and the extent of viable cells left in the culture after TCR-specific cytolysis was measured using the CellTiter Glo luminescence-based assay. The results are shown in FIG. 31.

Specific lysis was calculated from background subtracted values as:

$$\\ = [ \frac{( \begin{matrix}Tumor & TCR-T value - \\ ( TCR-T Value + Mean Tumor Value )\end{matrix} )}{TCR-T Value - ( TCR-T Value + Mean Tumor Value )} ] \times 100$$

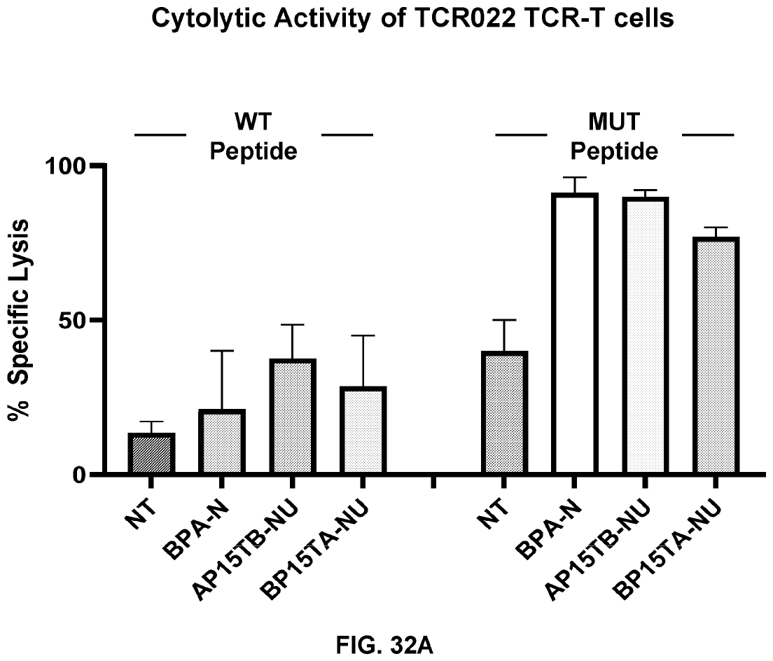

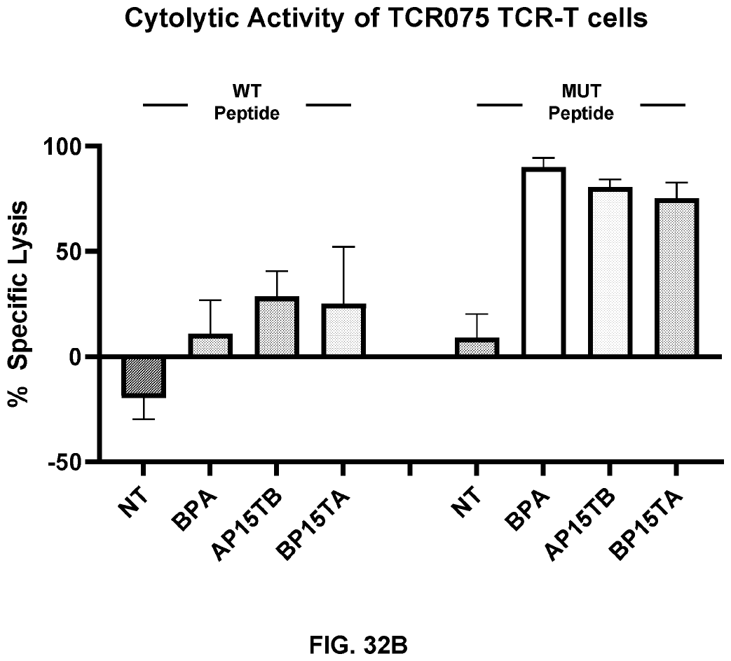

Cytolytic activity of TCR-T cells was also assessed for T cells electroporated with polycistronic plasmids encoding TCR022+/−mbIL15 or TCR075+/−mbIL15 generated as described above (overnight recovery+11 days first phase expansion+11 days expansion) and then harvested and frozen on Day 22. On experimental day, frozen Day 22 TCR-T cells were thawed and recovered for 3 days in media containing 3000 U/ml of IL-2. Meantime, Saos-2 cells were plated in 96 well plate. After overnight incubation, HLA*11:01 plasmid was transfected into the Saos-2 cells and on the following day, WT or MUT neoantigenic peptides (1 ug/ml) were loaded on the transfected Saos-2 cells for 2 hours. Then, the recovered TCR-T cells were incubated with the resulting Saos-2 cells overnight. After the overnight incubation, the remaining T cells were extensively washed, and the extent of viable cells left in the culture after TCR-specific cytolysis was measured using the CellTiter Glo luminescence-based assay. The results are shown in FIG. 32A-32B.

Specific lysis was calculated from background subtracted values as:

$$\\ = [ \frac{( \begin{matrix}Peptide loaded tumor & TCR-T value - \\ ( TCR-T Value + Mean Peptide loaded Tumor Value\end{matrix} )}{\begin{matrix}TCR-T Value - \\ ( TCR-T Value - Mean Peptide loaded tumor Value )\end{matrix}} ] \times 100$$

The cytolytic activity data demonstrated that mbIL15 TCR-T cells generated using the tricistronic system exhibited specific lytic activity against target tumor cells.

The long-term withdrawal (LTWD) assay was performed to examine the transgene expression, survival and activation of T cells cultured under cytokine-free conditions. The LTWD assay was performed as follows. The engineered T cells at Day 22 (post first phase and second phase expansion) were transferred to T25 flask and cultured for 4 weeks in cytokine-free media (50:50). 50% of media was exchanged every week. For the control TCR only (BA) groups, cells were treated with 300 U/ml IL-2 twice a week while exchanging the 50% of media. Flow data were acquired using an NovoCyte Quanteon flow cytometer system (Agilent) and analyzed with FlowJo software (version 10.7.1; TreeStar, Ashland, OR). (Data n=4, pooled from 2 independent experiments)

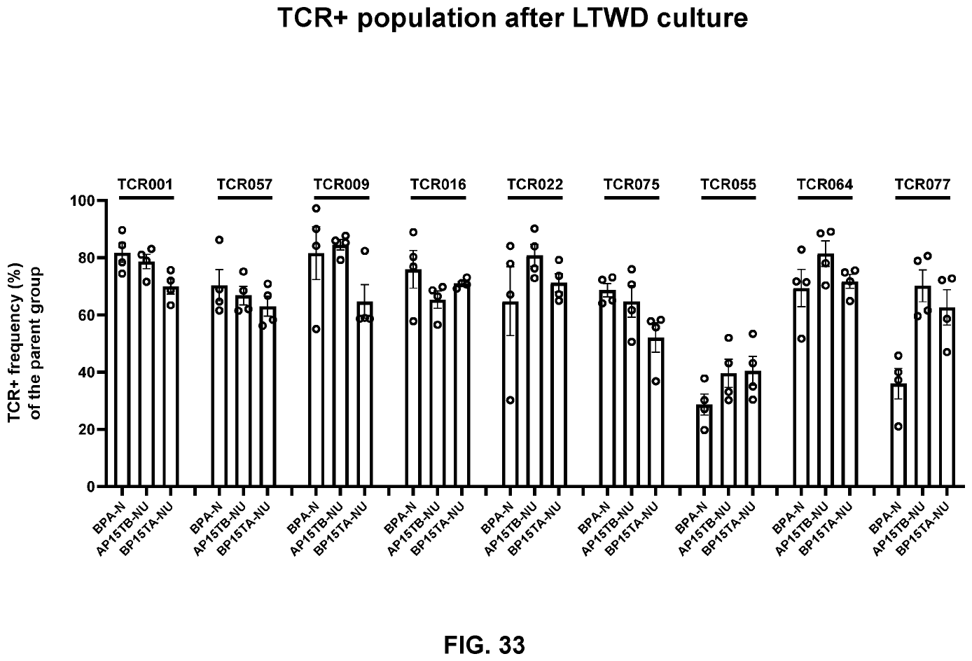

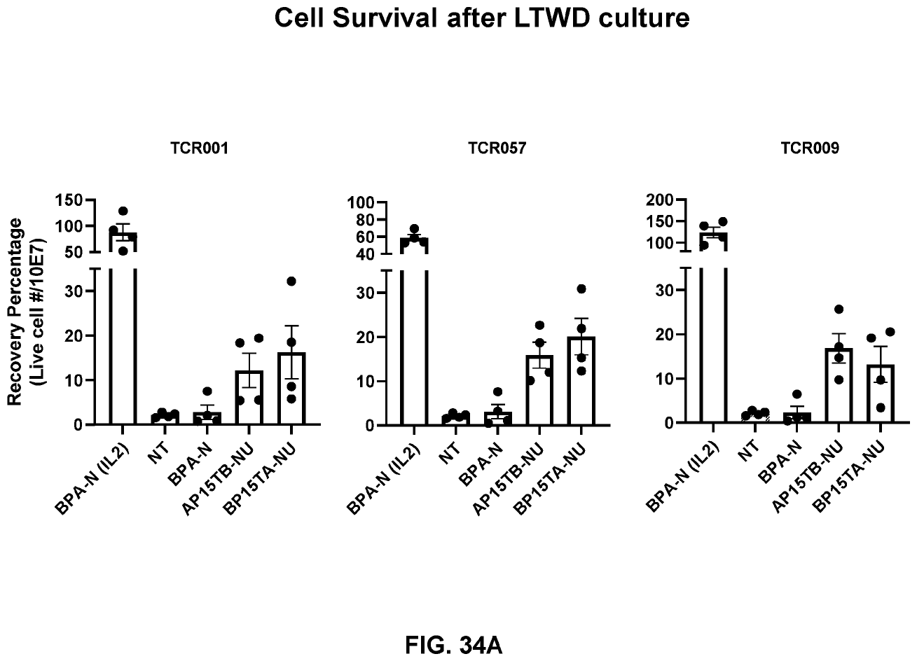

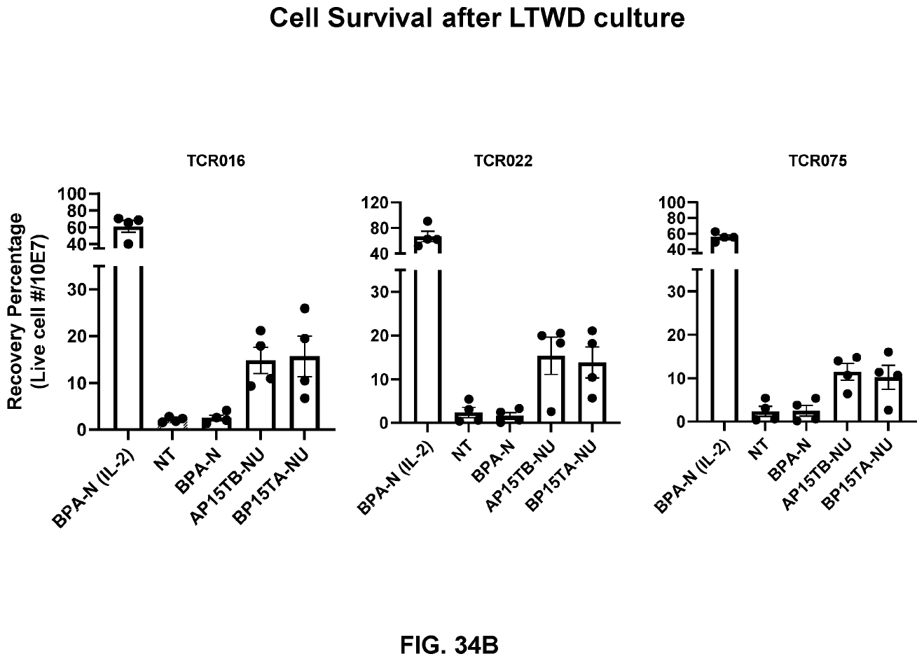

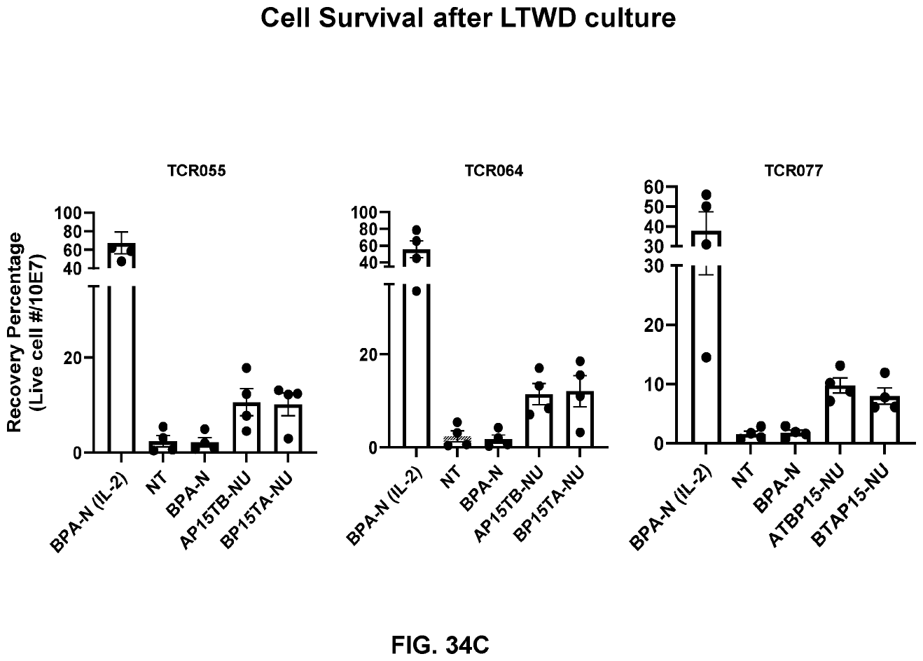

After 4 weeks LTWD incubation, the expression of mTCR was detected on CD3+ gated population with mouse TCR beta antibody (FIG. 33) and cell count and viability were accessed (FIG. 34A-34C). This mTCR expression and cell count data demonstrated no significant difference between mbIL15 TCR-T cells generated with the different polycistronic plasmids. The number of viable cells decreased after long-term cytokine withdrawal in all groups, but cells from the groups co-expressing mbIL15 and TCR survived 5-6 fold more compared to cells from the TCR only groups.

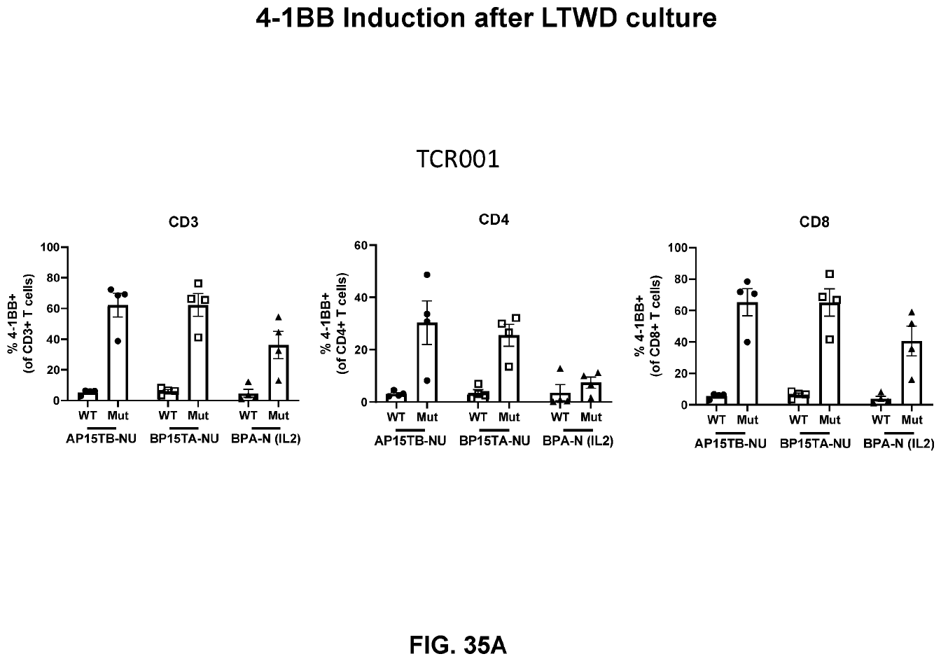

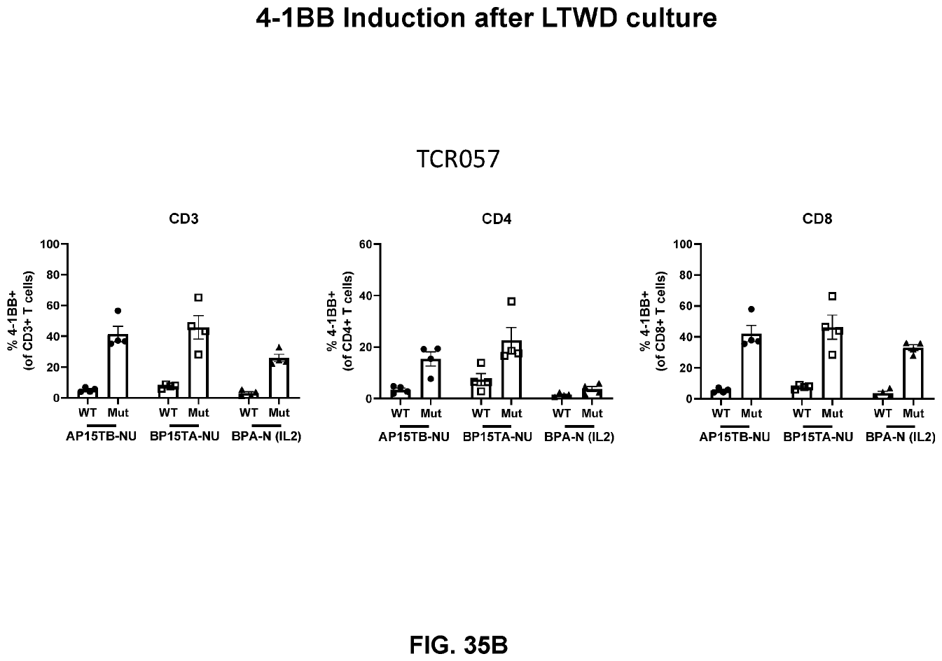

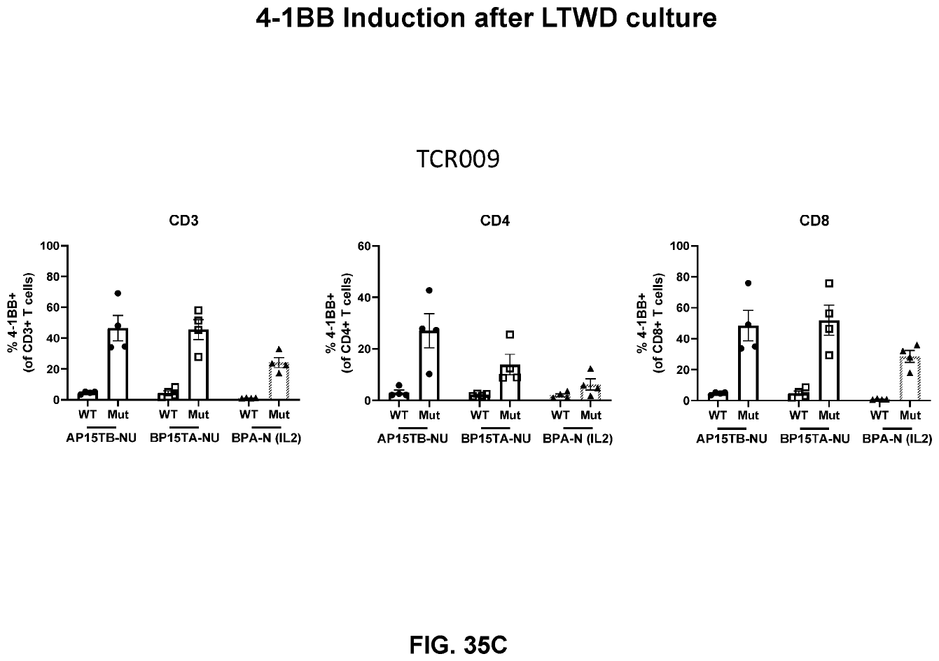

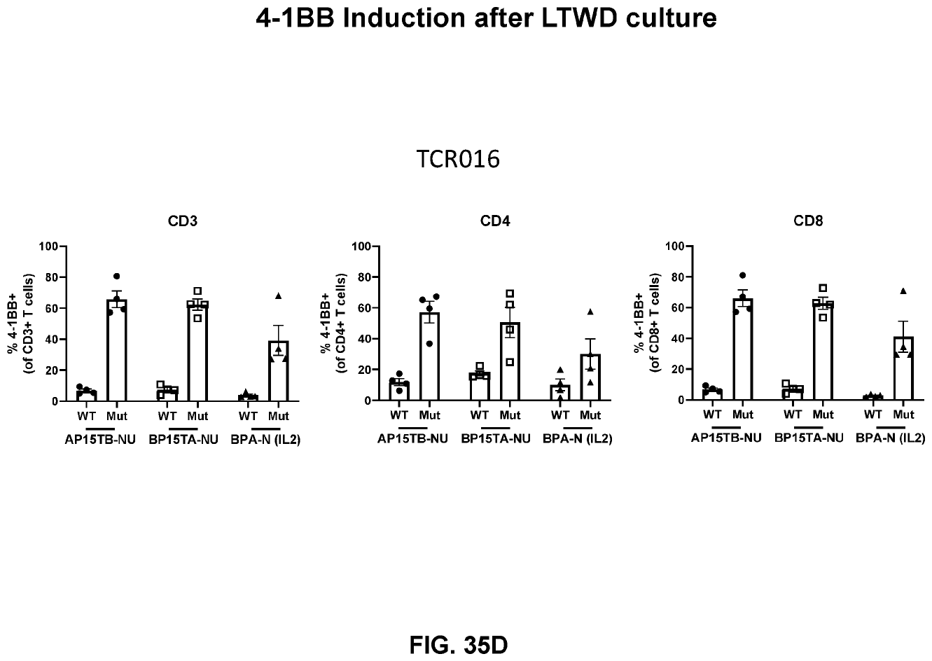

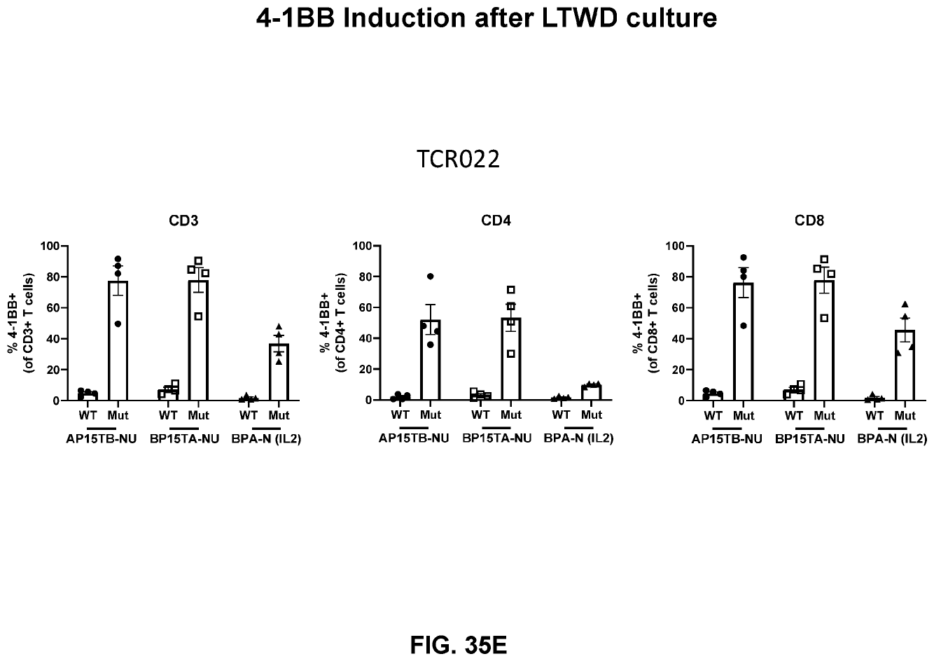

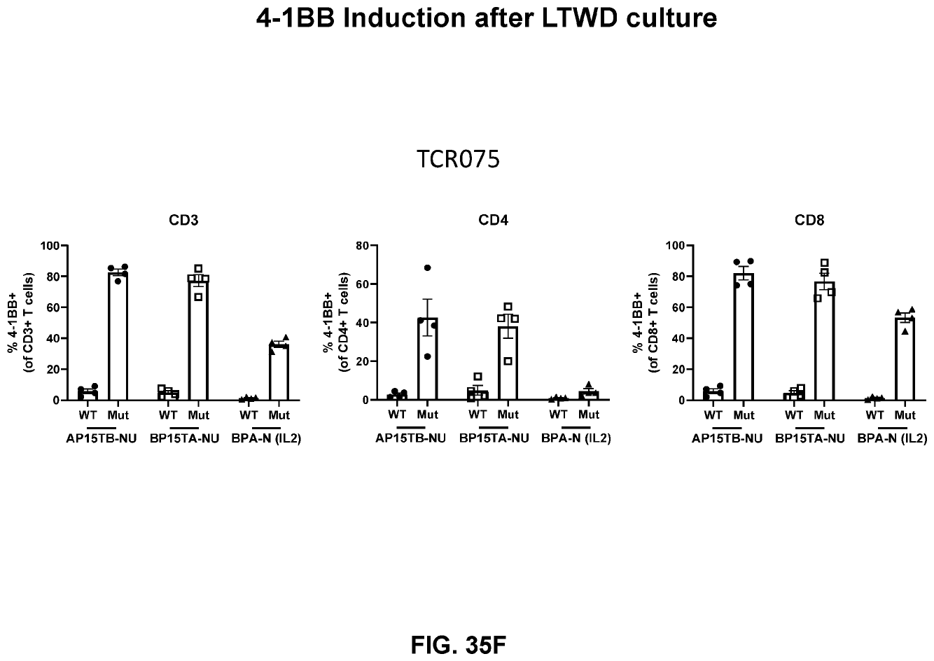

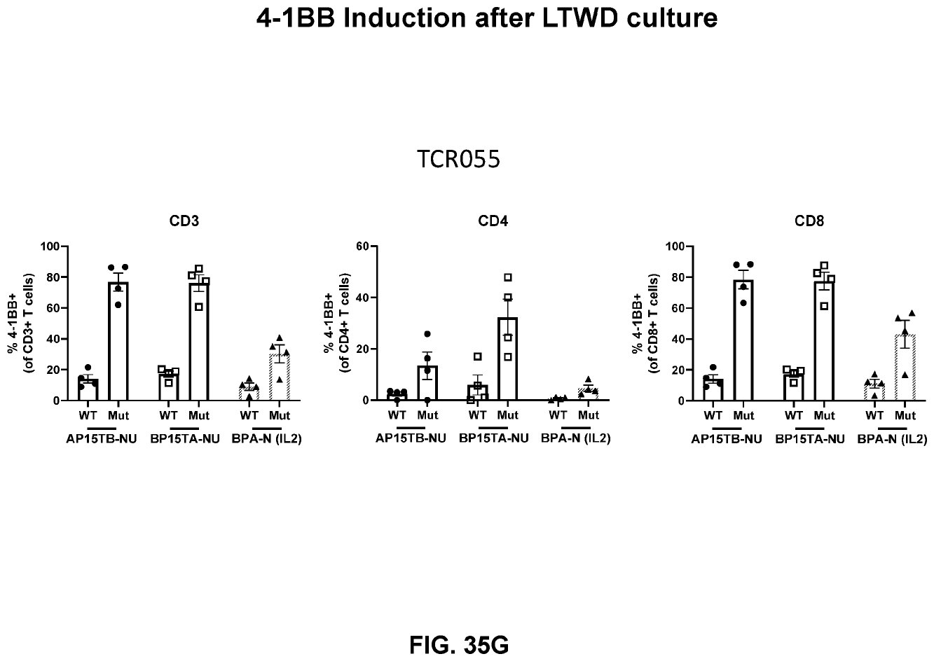

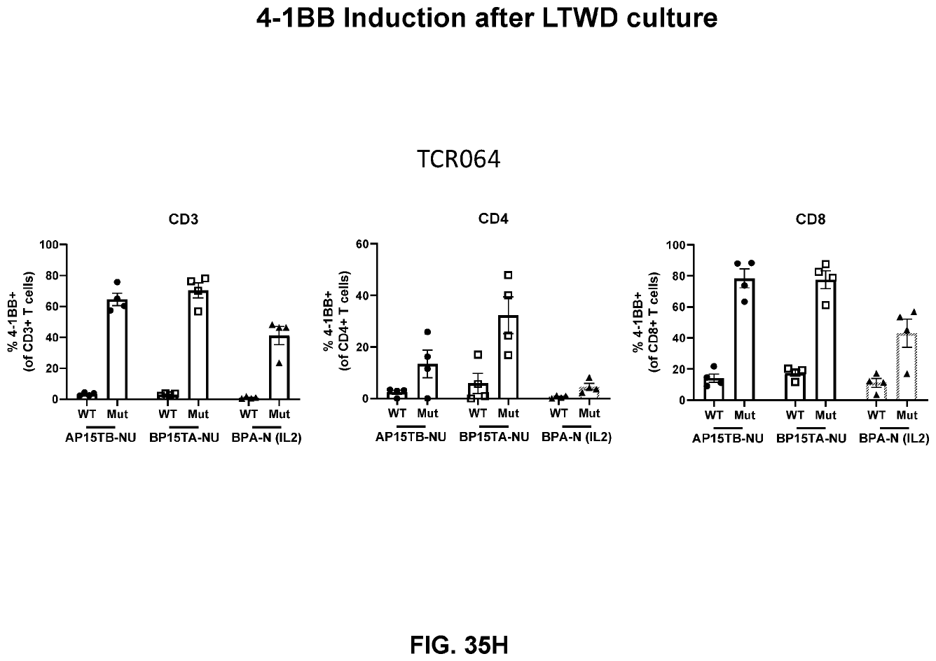

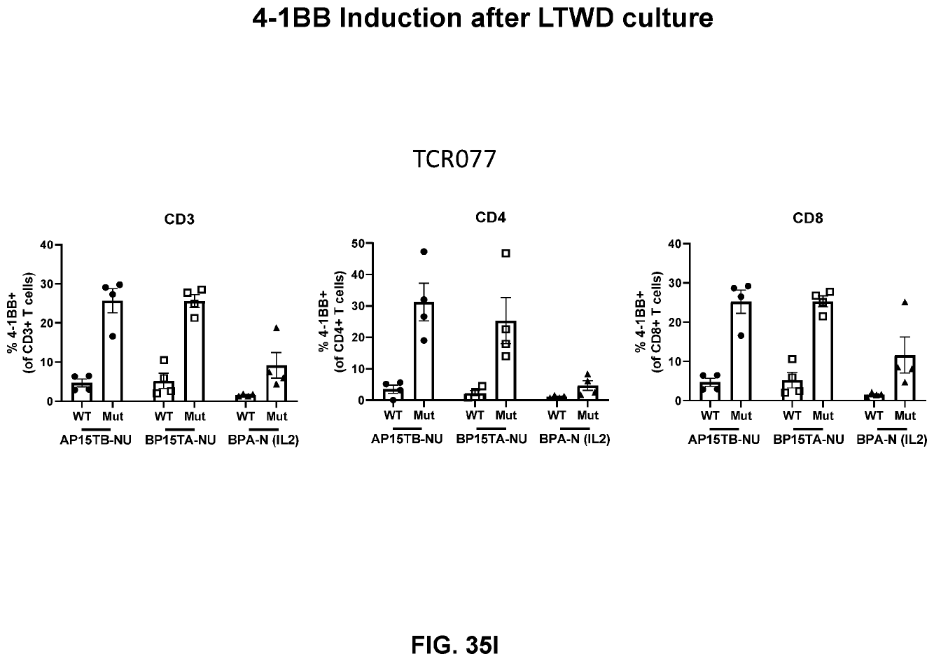

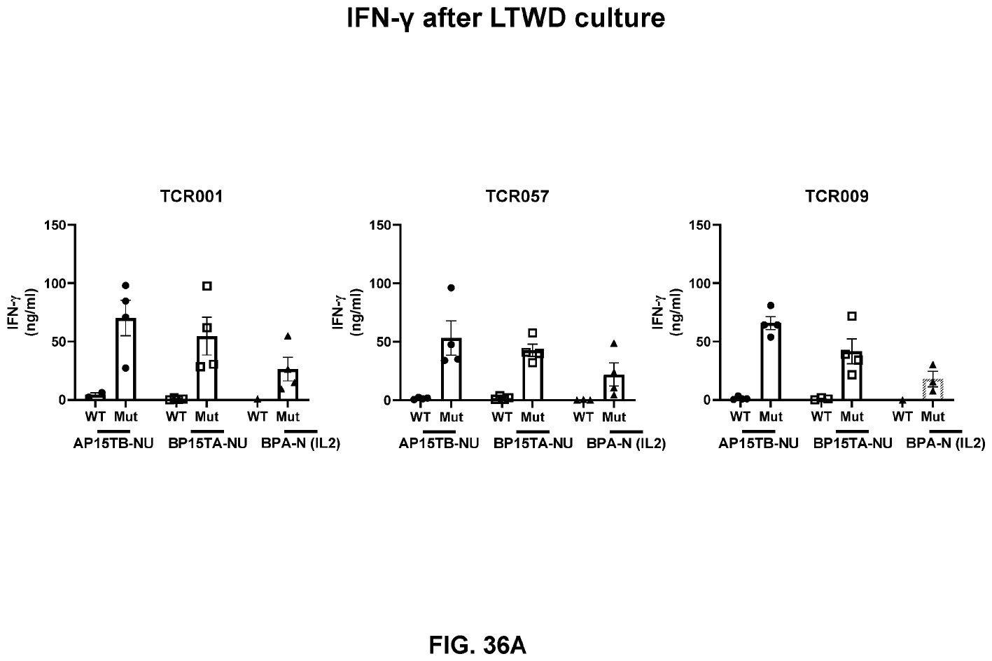

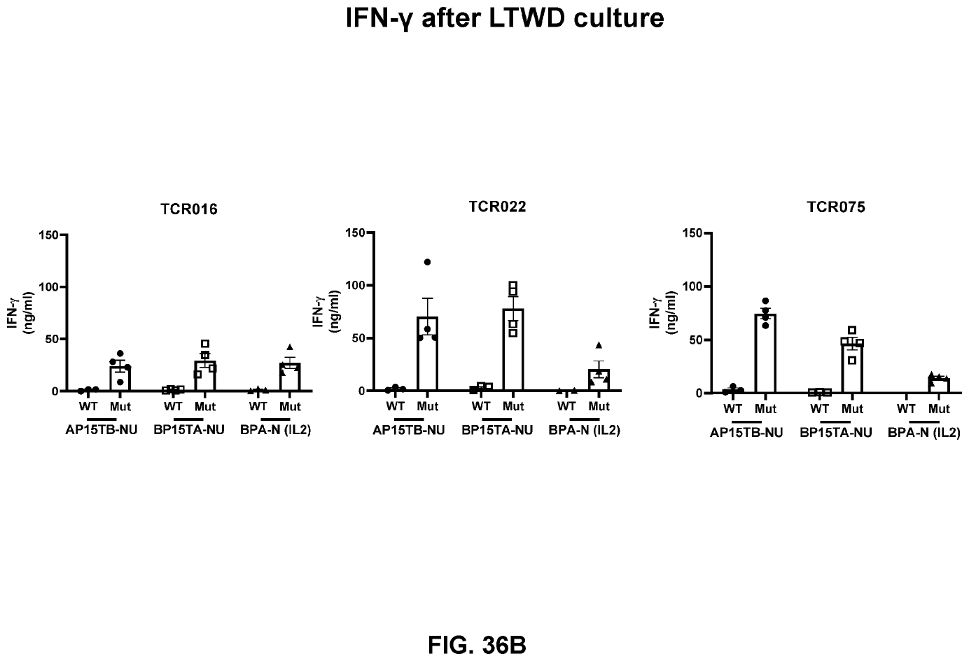

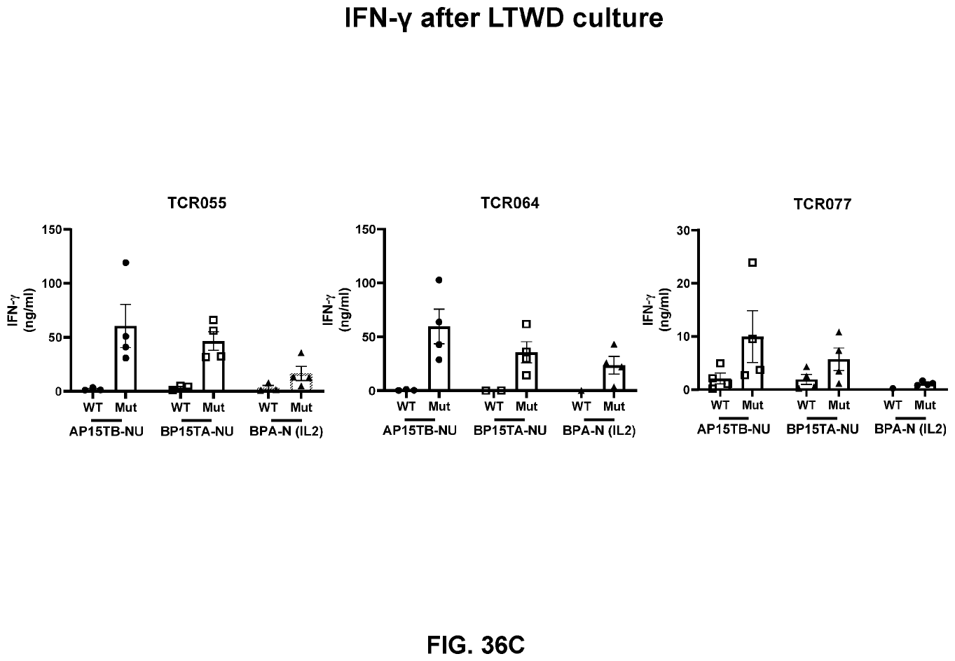

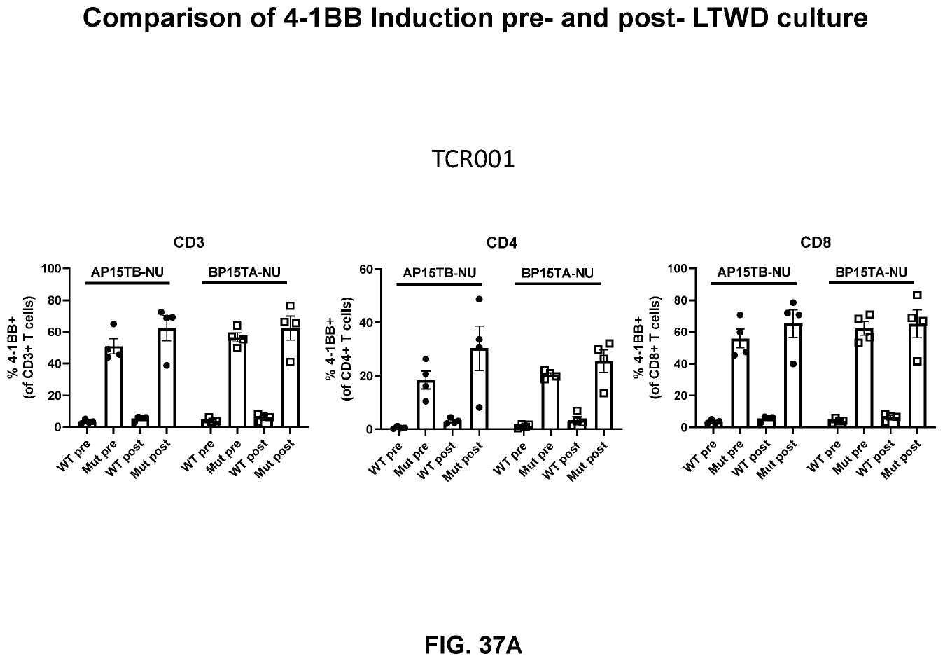

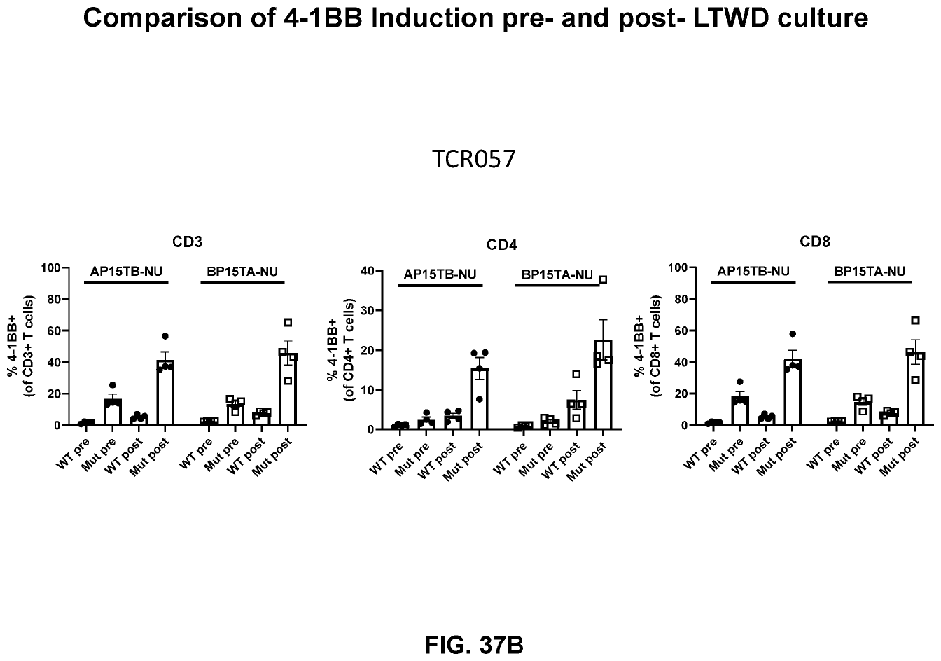

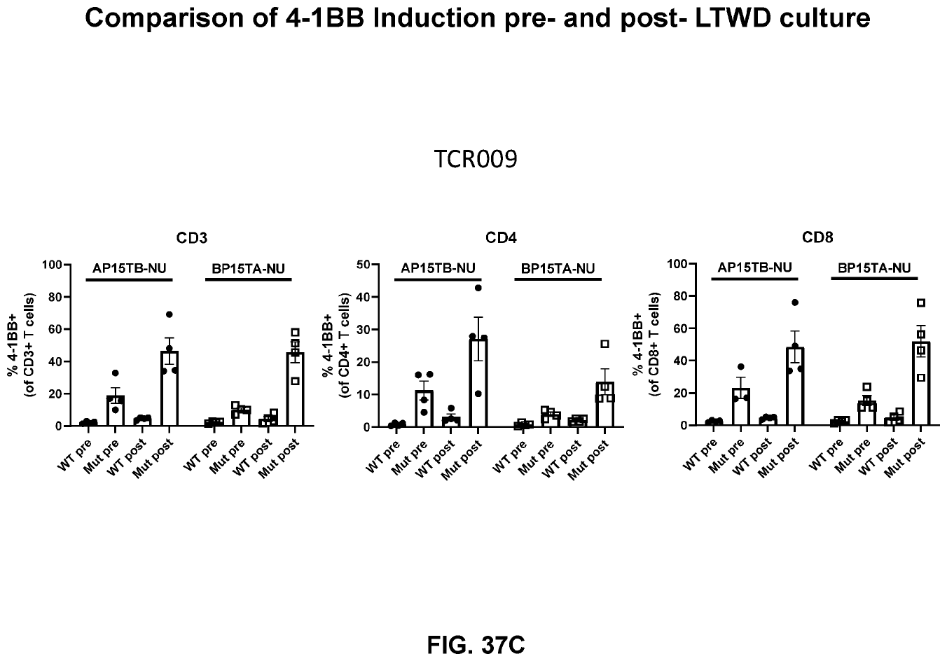

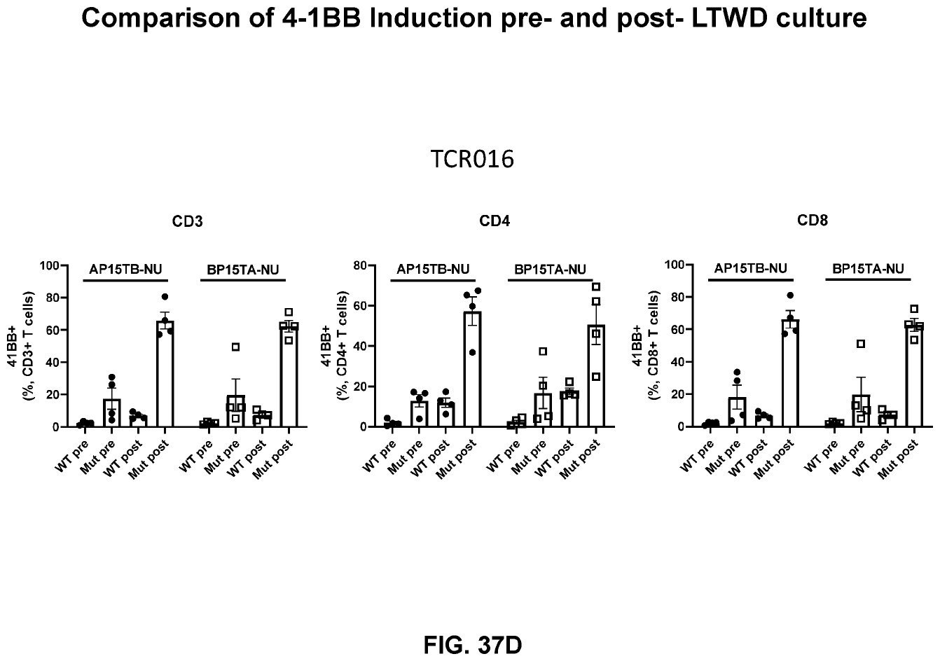

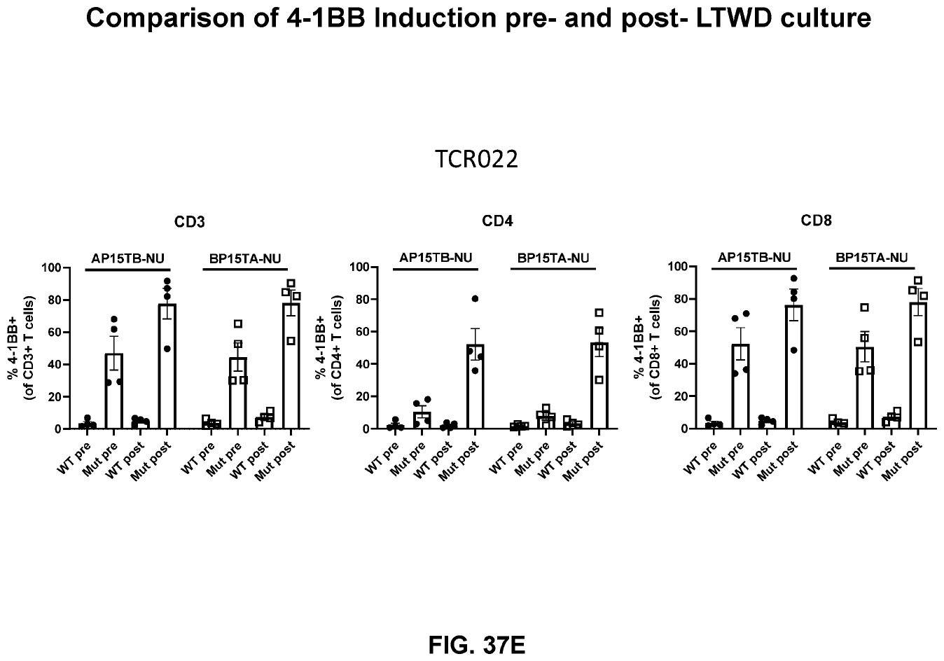

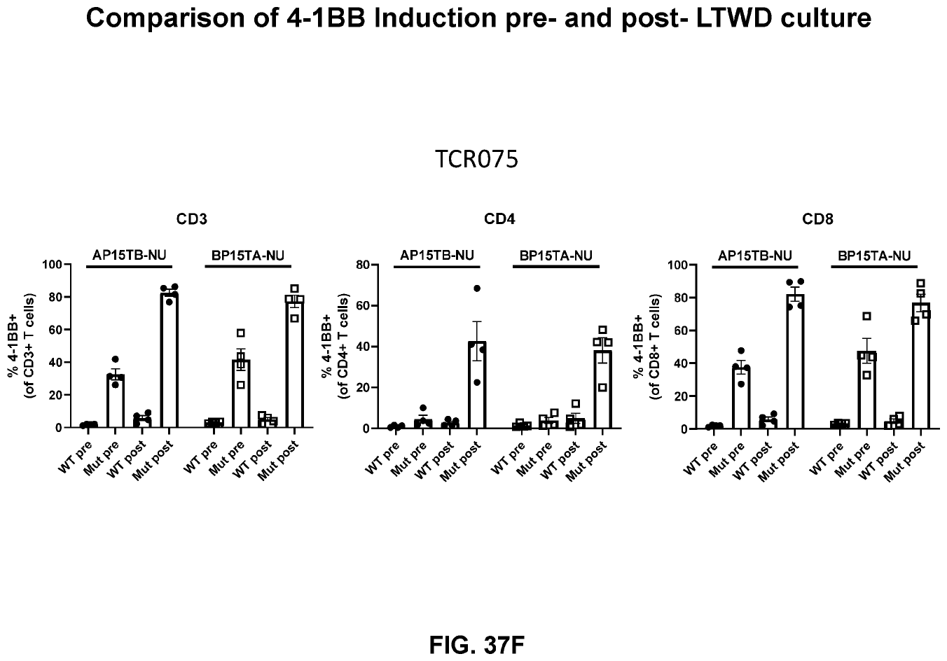

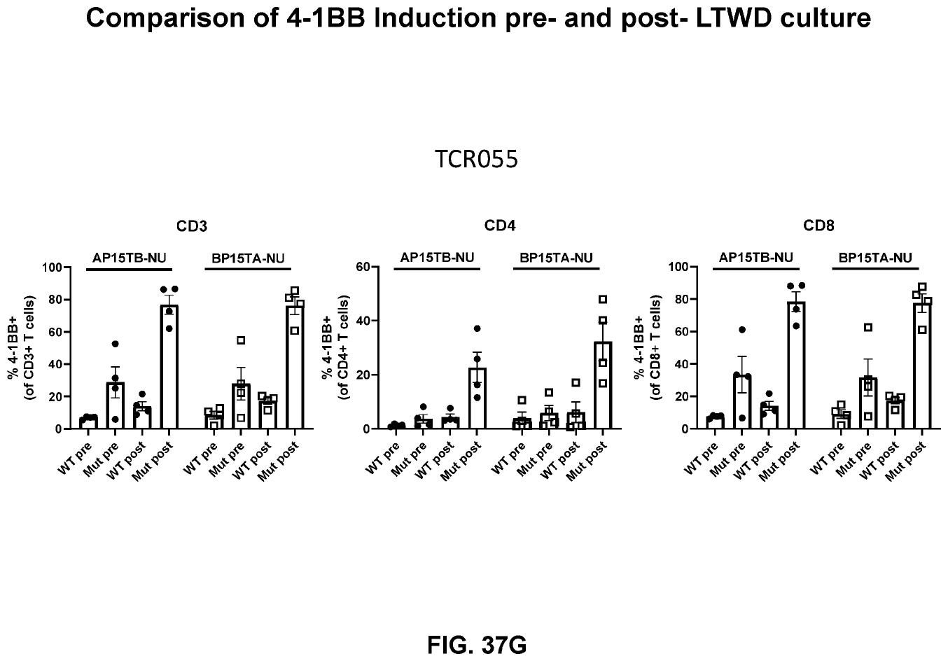

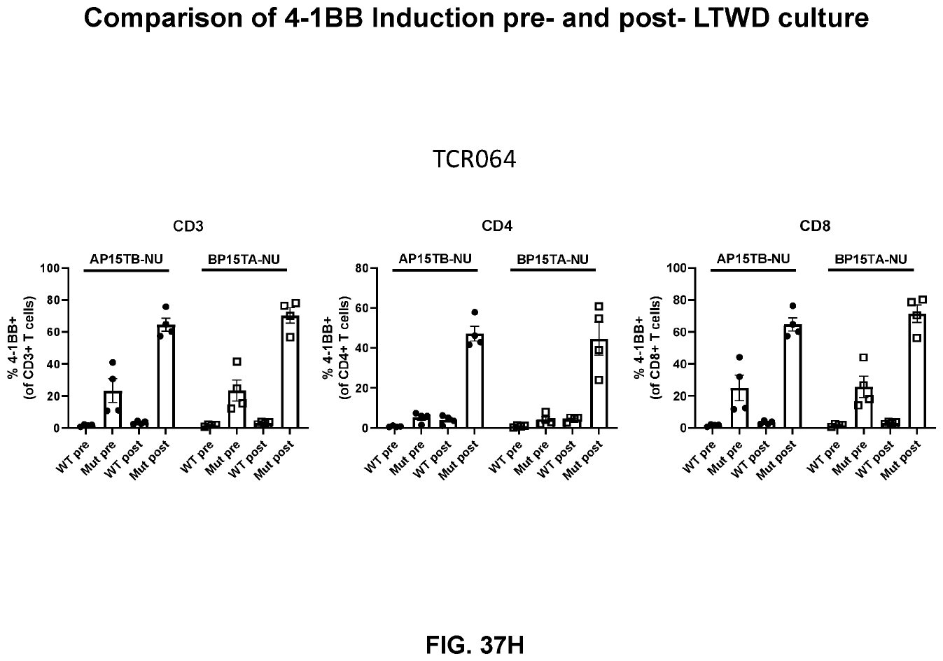

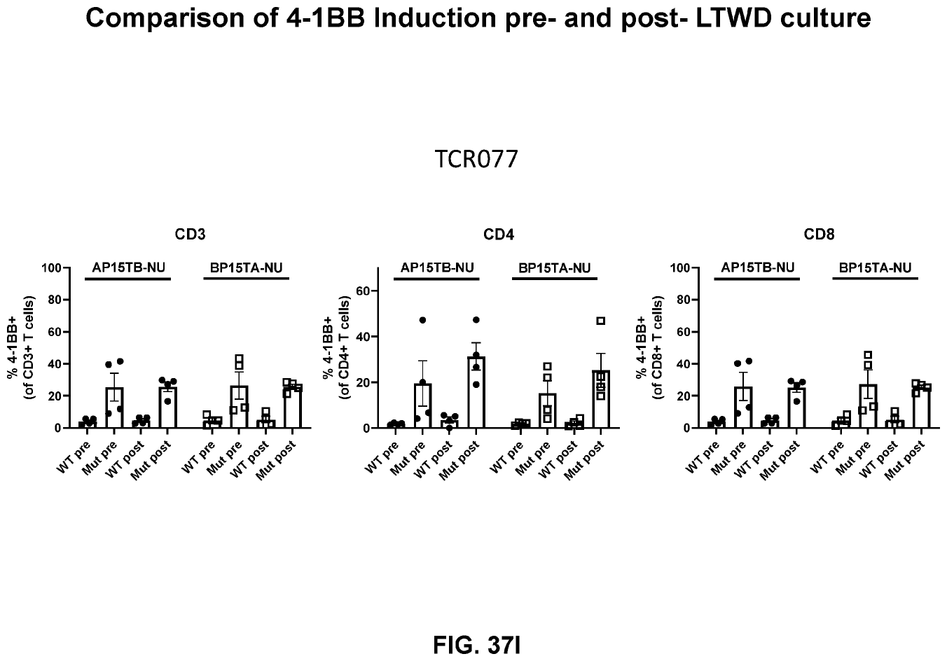

The activation of TCR-T cells after LTWD culture was assessed by 4-1BB induction and IFN-γ secretion after overnight co-culture with wild-type or mutant neoantigen pulsed DCs (HLA matched). As described above, induction of 4-1BB on CD3+CD8+ cells was detected with 4-1BB antibody (FIG. 35A-35I) and IFN-γ secretion was measured with the ELISA antibody pair (FIG. 36A-36C). A comparison of 4-1BB induction assessed for T cells harvested at Day 27 and after LTWD is shown in FIG. 37A-37I. These data demonstrate that mbIL15 TCR-T cells which survived LTWD culture are still functional and were more strongly activated than cells from TCR only groups. The data also demonstrate that after 4 week of cytokine withdrawal (LTWD), mbIL15 TCR-T cells showed even more potent induction of 4-1BB compared to those cells after the second expansion phase.

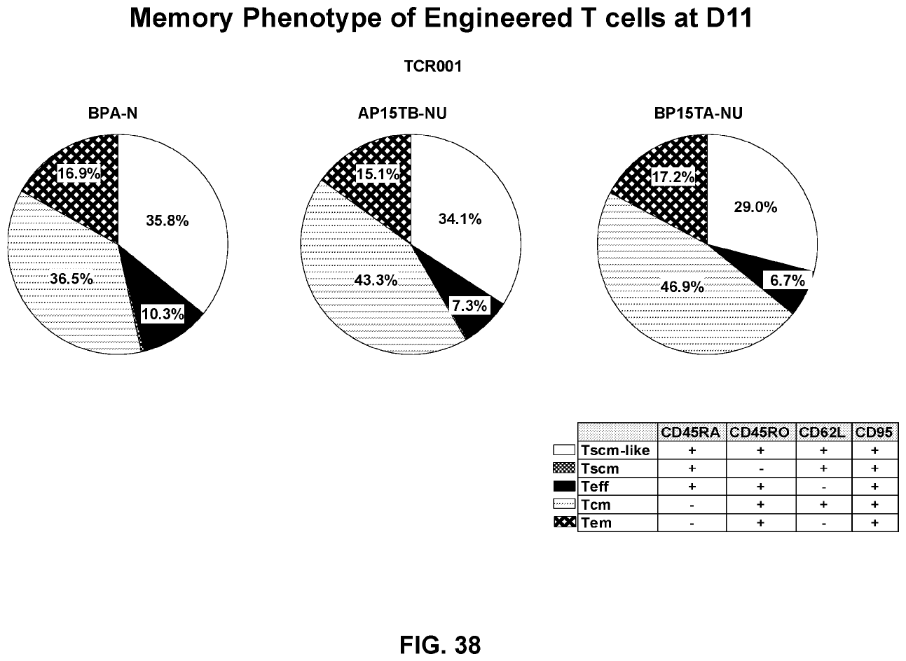

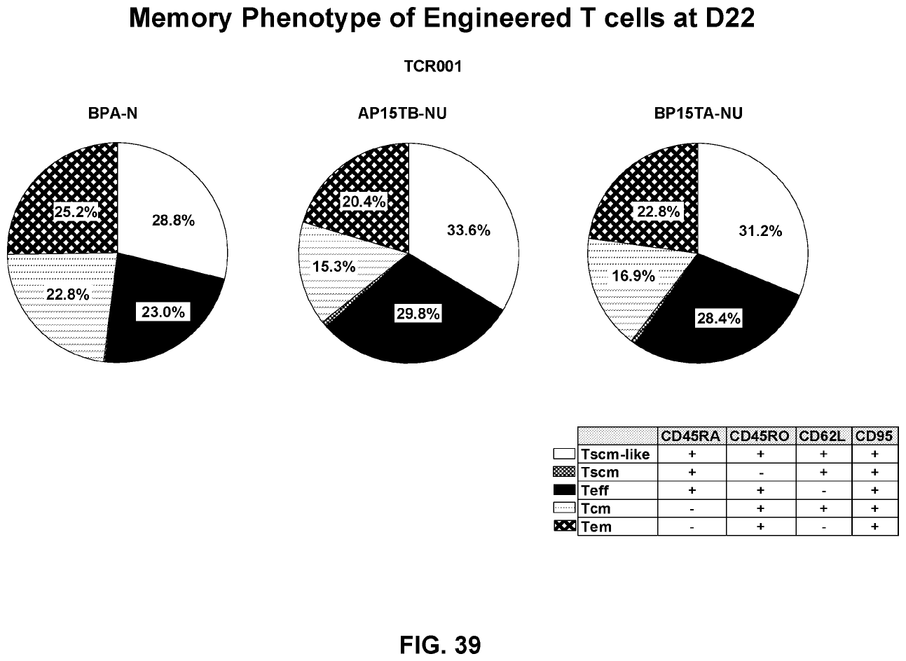

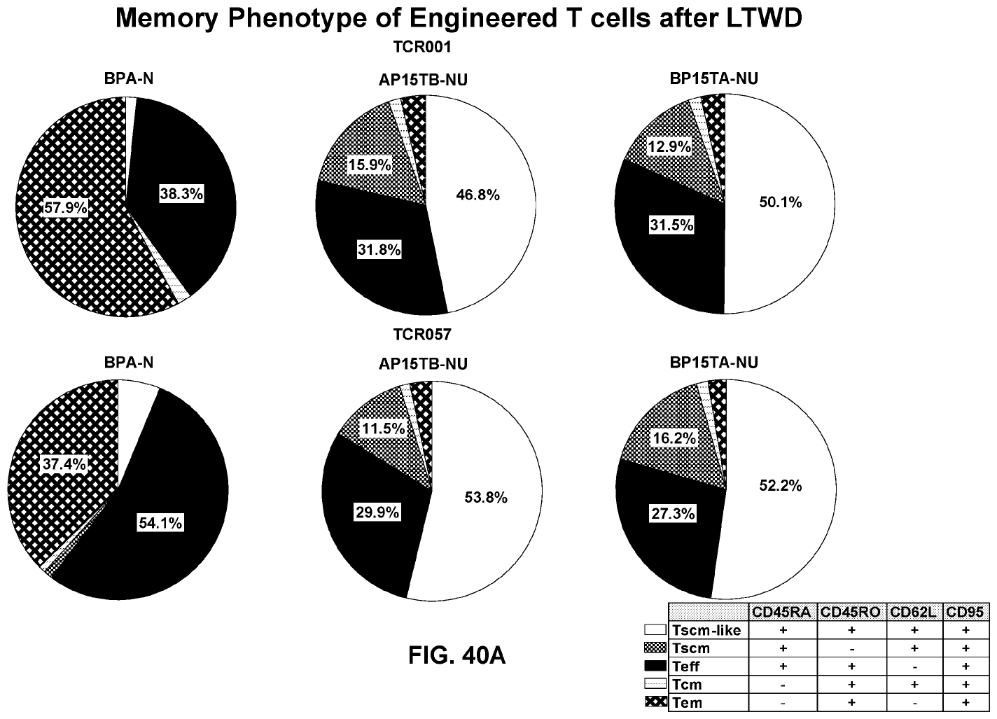

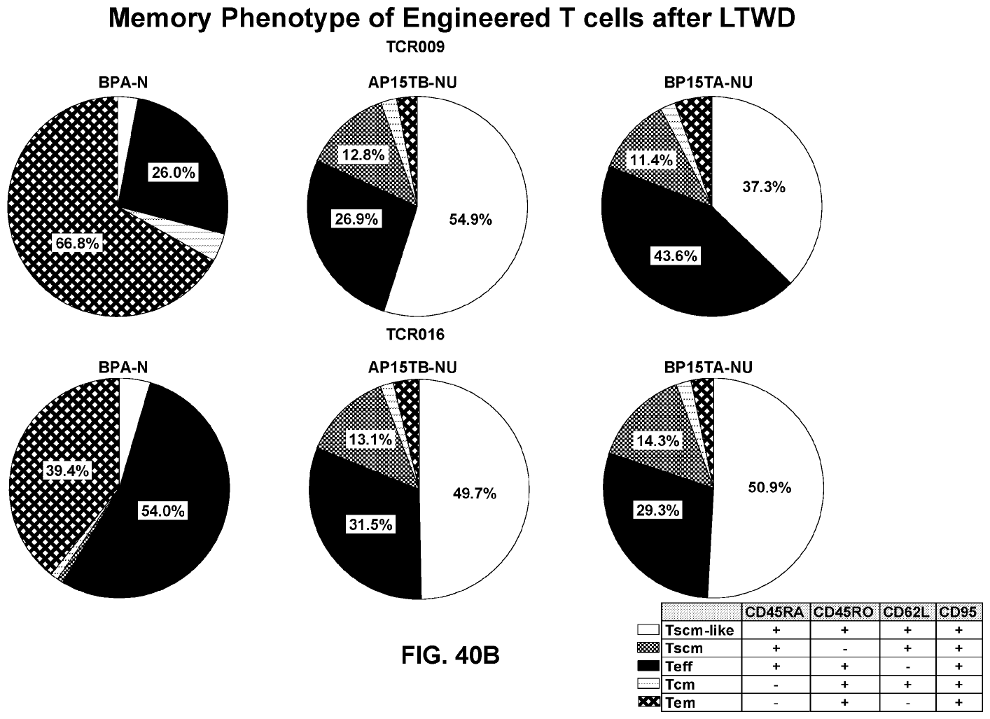

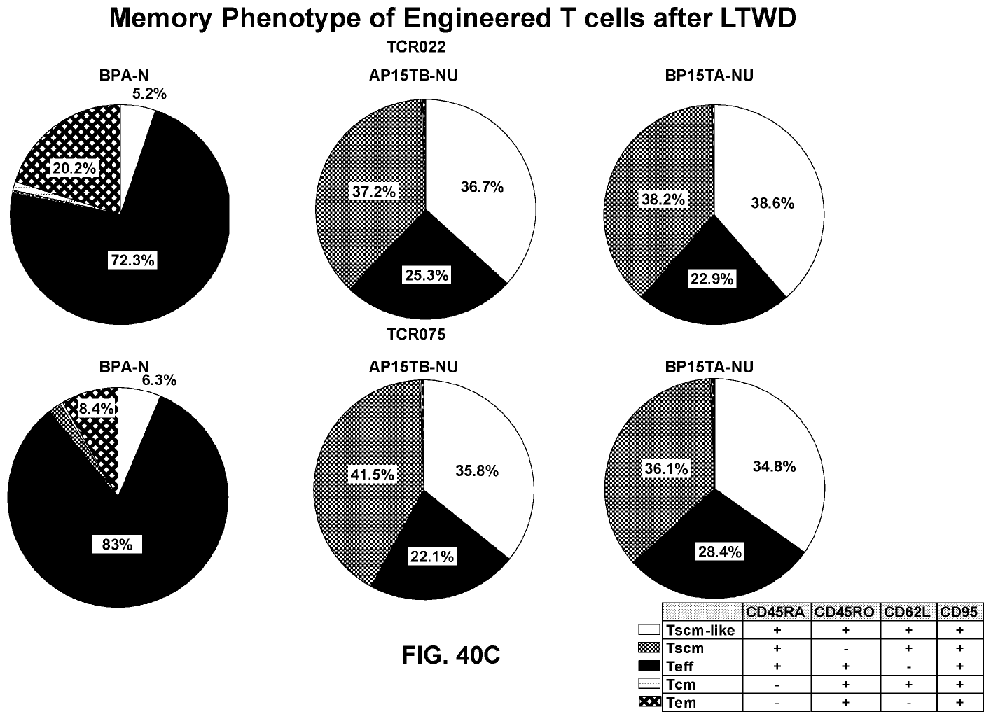

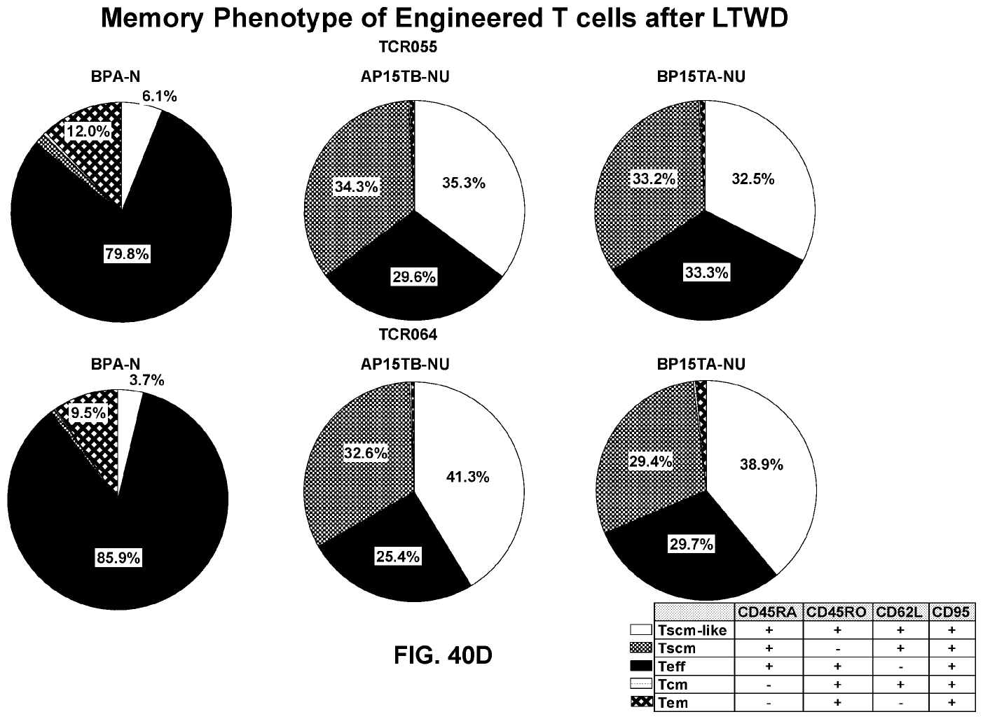

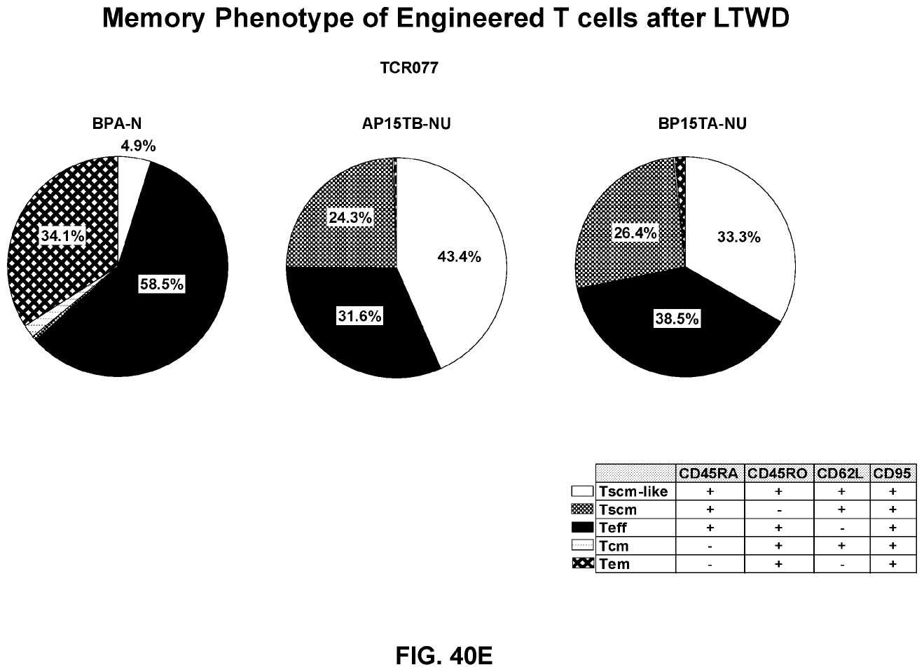

Memory phenotype of TCR-T cells electroporated with different polycistronic vectors was also assessed. T cell memory subsets are defined as: CD45RA+CD45RO+CD62L+CD95+=stem cell memory-like (Tscm-like); CD45RA+CD45RO−CD62L+CD95+=stem cell memory (Tscm); CD45RA-CD45RO+CD62L+CD95+=central memory (Tcm); CD45RA-CD45RO+CD62L-CD95+=effector memory (Tem). T cell effector (Teff) are defined as CD45RA+CD45RO+CD62L-CD95+. The data in Tables E13 and E14 and representative pie charts in FIGS. 38-40 show the mean frequency of live CD3+ T cell memory and effector subsets at day 11 post-expansion (Table E13 & FIG. 38), day 22 post-expansion (Table E14 & FIG. 39), and after 4 weeks of LTWD culture (FIGS. 40A-40E) in cells transposed with the tested plasmids.

TABLE E13
Memory phenotype of engineered T cells at D11.

| | | % Tscm- | % | % | % | % |
|---|---|---|---|---|---|---|
| Plasmid | | like | Tscm | Teff | Tcm | Tem |
| 1 | TCR001-BPA-N | 35.8 | 0.5 | 10.3 | 36.5 | 16.9 |
| | TCR001-AP15TB-NU | 34.1 | 0.2 | 7.3 | 43.3 | 15.1 |
| | TCR001-BP15TA-NU | 29.0 | nd | 6.7 | 46.9 | 17.2 |
| 2 | TCR057-BPA-N | 37.1 | 0.4 | 12.3 | 31.6 | 18.6 |
| | TCR057-AP15TB-NU | 33.7 | 0.1 | 7.7 | 43.5 | 15.0 |
| | TCR057-BP15TA-NU | 31.4 | 0.2 | 6.8 | 45.5 | 16.1 |
| 3 | TCR009-BPA-N | 38.3 | 0.6 | 11.8 | 29.9 | 19.4 |
| | TCR009-AP15TB-NU | 34.8 | 0.1 | 7.1 | 44.0 | 14.0 |
| | TCR009-BP15TA-NU | 28.5 | 0.2 | 6.3 | 47.7 | 17.3 |
| 4 | TCR016-BPA-N | 40.2 | 0.6 | 12.6 | 30.0 | 16.6 |
| | TCR016-AP15TB-NU | 32.8 | 0.4 | 6.8 | 44.8 | 15.2 |
| | TCR016-BP15TA-NU | 32.1 | 0.3 | 7.0 | 45.1 | 15.5 |
| 5 | TCR022-BPA-N | 58.1 | 2.9 | 7.5 | 25.6 | 5.9 |
| | TCR022-AP15TB-NU | 55.1 | 0.8 | 2.4 | 38.7 | 3.0 |
| | TCR022-BP15TA-NU | 56.9 | 0.7 | 2.3 | 37.4 | 2.7 |
| 6 | TCR075-BPA-N | 59.7 | 2.8 | 8.5 | 23.9 | 5.1 |
| | TCR075-AP15TB-NU | 53.5 | 0.7 | 2.1 | 40.6 | 3.1 |
| | TCR075-BP15TA-NU | 56.8 | 0.8 | 2.5 | 37.2 | 2.7 |
| 7 | TCR055-BPA-N | 55.6 | 2.5 | 8.2 | 27.8 | 5.9 |
| | TCR055-AP15TB-NU | 56.0 | 0.9 | 2.5 | 38.1 | 2.5 |
| | TCR055-BP15TA-NU | 56.6 | 0.8 | 2.5 | 37.3 | 2.8 |
| 8 | TCR064-BPA-N | 57.3 | 3.3 | 8.2 | 25.5 | 5.7 |
| | TCR064-AP15TB-NU | 53.3 | 0.8 | 2.5 | 40.1 | 3.3 |
| | TCR064-BP15TA-NU | 54.0 | 0.6 | 2.4 | 39.6 | 3.4 |
| 9 | TCR077-BPA-N | 63.8 | 3.0 | 5.3 | 22.0 | 5.8 |
| | TCR077-AP15TB-NU | 50.7 | 0.2 | 3.7 | 41.2 | 4.2 |
| | TCR077-BP15TA-NU | 50.7 | 0.2 | 4.3 | 40.4 | 4.4 |

TABLE E14
Memory phenotype of engineered T cells at D22.

| | | % Tscm- | % | % | % | % |
|---|---|---|---|---|---|---|
| Plasmid | | like | Tscm | Teff | Tcm | Tem |
| 1 | TCR001-BPA-N | 28.8 | 0.2 | 23.0 | 22.8 | 25.2 |
| | TCR001-AP15TB-NU | 33.6 | 0.9 | 29.8 | 15.3 | 20.4 |
| | TCR001-BP15TA-NU | 31.2 | 0.7 | 28.4 | 16.9 | 22.8 |
| 2 | TCR057-BPA-N | 31.2 | 0.2 | 22.7 | 23.1 | 22.8 |
| | TCR057-AP15TB-NU | 33.5 | 0.4 | 28.1 | 18.0 | 20.0 |
| | TCR057-BP15TA-NU | 34.4 | 0.7 | 28.5 | 16.5 | 19.9 |
| 3 | TCR009-BPA-N | 26.3 | 0.1 | 19.0 | 27.7 | 26.9 |
| | TCR009-AP15TB-NU | 34.7 | 0.2 | 29.4 | 15.3 | 19.4 |
| | TCR009-BP15TA-NU | 27.5 | 0.8 | 32.9 | 12.7 | 26.1 |
| 4 | TCR016-BPA-N | 30.7 | 0.1 | 22.1 | 23.3 | 23.8 |
| | TCR016-AP15TB-NU | 35.3 | 0.8 | 29.6 | 14.1 | 20.2 |
| | TCR016-BP15TA-NU | 33.4 | 0.8 | 28.9 | 14.7 | 22.2 |
| 5 | TCR022-BPA-N | 28.7 | 0.1 | 15.3 | 31.8 | 24.1 |
| | TCR022-AP15TB-NU | 32.3 | 0.7 | 22.1 | 25.1 | 19.8 |
| | TCR022-BP15TA-NU | 30.4 | 0.5 | 22.8 | 24.1 | 22.2 |
| 6 | TCR075-BPA-N | 26.9 | 0.2 | 15.6 | 33.4 | 23.9 |
| | TCR075-AP15TB-NU | 31.7 | 0.7 | 22.6 | 22.3 | 22.7 |
| | TCR075-BP15TA-NU | 31.4 | 0.5 | 23.9 | 22.2 | 22.0 |
| 7 | TCR055-BPA-N | 22.9 | 0.2 | 14.9 | 35.1 | 26.9 |
| | TCR055-AP15TB-NU | 28.8 | 0.5 | 27.5 | 19.7 | 23.5 |
| | TCR055-BP15TA-NU | 28.8 | 0.4 | 25.7 | 20.2 | 24.9 |
| 8 | TCR064-BPA-N | 24.1 | 0.1 | 13.9 | 34.3 | 27.6 |
| | TCR064-AP15TB-NU | 28.7 | 0.3 | 21.1 | 25.8 | 24.1 |
| | TCR064-BP15TA-NU | 28.8 | 0.6 | 22.5 | 23.6 | 24.5 |
| 9 | TCR077-BPA-N | 34.3 | 0.3 | 27.8 | 19.2 | 18.4 |
| | TCR077-AP15TB-NU | 37.8 | 2.2 | 34.8 | 10.7 | 14.6 |
| | TCR077-BP15TA-NU | 37.8 | 1.9 | 36.4 | 10.1 | 13.8 |

Memory phenotype data shows the kinetics of TCR-T memory and effector differentiation. The addition of mbIL15 to TCR-T cells resulted in changes to the memory phenotype in the expanded product to contain fewer central memory cells (Tcm) and more effector (Teff) and stem cell memory (Tscm) populations relative to conventional TCR-T cells. After 4 weeks of culture in presence of EL-2, TCR-T cells predominantly differentiated into Teff cells. TCR-T cells expressing mbIL15 cultured for 4 weeks in the absence of cytokines differentiated into 3 main subsets: Teff, Tscm-like and Tscm cells. These results suggest that mbIL15 is sufficient to guide T cell differentiation to the Tscm phenotype.

Conclusions: mbIL15 TCR-T cells were successfully generated using 18 different constructs (2 different orientations; AP15 TB and BP15TA X 9 TCRs). The addition of mbIL15 to TCR-T cells resulted in changes to the memory phenotype in the expanded product to contain fewer central memory cells (Tcm) and more effector (Teff) and stem cell memory (Tscm) populations relative to conventional TCR-T cells. Furthermore, long-term withdrawal of cytokine support (LTWD) demonstrated survival of a fraction of mbIL15 TCR-T cells which was significantly higher than survival of TCR-T cells lacking mbIL15. Functional and phenotypic evaluation of the persistent mbIL15 TCR-T cells revealed that they retained their functional neoantigen specificity and potency while displaying a preponderance of Tscm TCR-T cells capable of regenerating TCR-T cell effector pools. This suggested that mbIL15 TCR-T cells could likely establish long-lived tumor-specific TCR-T cells that potentially overcome suppression by the tumor microenvironment or other negative regulators. This non-clinical data supports clinical application of mbIL15 TCR-T cell platform and provides evidence that this strategy could result in improved efficacy for cancer treatment.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes. Other embodiments are within the following claims.

What is claimed:

1. A recombinant vector comprising a polycistronic expression cassette, wherein the polycistronic expression cassette comprises a transcriptional regulatory element operably linked to a polycistronic polynucleotide that comprises:
a. a first polynucleotide sequence that encodes a T cell receptor (TCR) alpha chain comprising an alpha chain variable (Vα) region and an alpha chain constant (Cα) region;
b. a second polynucleotide sequence that comprises a first 2A element;
c. a third polynucleotide sequence that encodes a TCR beta chain comprising a beta chain variable (Vβ) region and a beta chain constant (Cβ) region;
d. a fourth polynucleotide sequence that comprises a second 2A element; and
e. a fifth polynucleotide sequence that encodes a fusion protein that comprises IL-15, or a functional fragment or functional variant thereof, and IL-15Rα, or a functional fragment or functional variant thereof.

2. The recombinant vector of claim 1, wherein either or both of the first 2A element and the second 2A element, independently, is a P2A element, a T2A element, an F2A element, or an E2A element.

3. The recombinant vector of claim 1, wherein the first 2A element is a P2A element.

4. The recombinant vector of claim 2 or 3, wherein the P2A element comprises a polynucleotide sequence that encodes the amino acid sequence of SEQ ID NO: 18 or 20, or the amino acid sequence of SEQ ID NO: 18 or 20 comprising 1, 2, or 3 amino acid modifications.

5. The recombinant vector of claim 2 or 3, wherein the P2A element comprises a polynucleotide sequence at least 75%, 80%, 85%, 90%, 95%, 96%, 97%, 98%, 99%, or 100% identical to the polynucleotide sequence of SEQ ID NO: 19 or 21.

6. The recombinant vector of any one of claims 1 or 3-5, wherein the second 2A element is a T2A element.

7. The recombinant vector of claim 2 or 6, wherein the T2A element comprises a polynucleotide sequence that encodes the amino acid sequence of SEQ ID NO: 22 or 24, or the amino acid sequence of SEQ ID NO: 22 or 24 comprising 1, 2, or 3 amino acid modifications.

8. The recombinant vector of claim 2 or 6, wherein the T2A element comprises a polynucleotide sequence at least 75%, 80%, 85%, 90%, 95%, 96%, 97%, 98%, 99%, or 100% identical to the polynucleotide sequence of SEQ ID NO: 23 or 25.

9. The recombinant vector of any one of claims 1-8, wherein either or both of the second polynucleotide sequence and the fourth polynucleotide sequence, independently, encode a furin recognition site.

10. The recombinant vector of claim 9, wherein the furin recognition site comprises the amino acid sequence of SEQ ID NO: 2 or 4, or the amino acid sequence of SEQ ID NO: 2 or 4 comprising 1, 2, or 3 amino acid modifications.

11. The recombinant vector of claim 9, wherein the furin recognition site is encoded by the polynucleotide sequence of SEQ ID NO: 3 or 5 or the polynucleotide sequence of SEQ ID NO: 3 or 5 comprising 1, 2, or 3 nucleotide modifications.

12. The recombinant vector of any one of claims 1-11, wherein the second polynucleotide sequence comprises a polynucleotide sequence that encodes the amino acid sequence of SEQ ID NO: 10, or the amino acid sequence of SEQ ID NO: 10 comprising 1, 2, or 3 amino acid modifications.

13. The recombinant vector of any one of claims 1-11, wherein the second polynucleotide sequence comprises a polynucleotide sequence at least 75%, 80%, 85%, 90%, 95%, 96%, 97%, 98%, 99%, or 100% identical to the polynucleotide sequence of SEQ ID NO: 11.

14. The recombinant vector of any one of claims 1-13, wherein the fourth polynucleotide sequence comprises a polynucleotide sequence that encodes the amino acid sequence of SEQ ID NO: 12, or the amino acid sequence of SEQ ID NO: 12 comprising 1, 2, or 3 amino acid modifications.

15. The recombinant vector of any one of claims 1-13, wherein the fourth polynucleotide sequence comprises a polynucleotide sequence at least 75%, 80%, 85%, 90%, 95%, 96%, 97%, 98%, 99%, or 100% identical to the polynucleotide sequence of SEQ ID NO: 13.

16. The recombinant vector of any one of claims 1-15, wherein the IL-15, or functional fragment or functional variant thereof, comprises an amino acid sequence at least 90%, 95%, 96%, 97%, 98%, 99%, or 100% identical to the amino acid sequence of SEQ ID NO: 76.

17. The recombinant vector of any one of claims 1-15, wherein the IL-15, or functional fragment or functional variant thereof, is encoded by a polynucleotide sequence at least 75%, 80%, 85%, 90%, 95%, 96%, 97%, 98%, 99%, or 100% identical to the polynucleotide sequence of SEQ ID NO: 77.

18. The recombinant vector of any one of claims 1-15, wherein the IL-15Rα, or functional fragment or functional variant thereof, comprises an amino acid sequence at least 90%, 95%, 96%, 97%, 98%, 99%, or 100% identical to the amino acid sequence of SEQ ID NO: 78.

19. The recombinant vector of any one of claims 1-15, wherein the IL-15Rα, or functional fragment or functional variant thereof, is encoded by a polynucleotide sequence at least 75%, 80%, 85%, 90%, 95%, 96%, 97%, 98%, 99%, or 100% identical to the polynucleotide sequence of SEQ ID NO: 79.

20. The recombinant vector of any one of claims 1-19, wherein the IL-15, or functional fragment or functional variant thereof, is operably linked to the IL-15Rα, or functional fragment or functional variant thereof, via a peptide linker.

21. The recombinant vector of claim 20, wherein the peptide linker comprises the amino acid sequence of SEQ ID NO: 81, or the amino acid sequence of SEQ ID NO: 81 comprising 1, 2, or 3 amino acid modifications.

22. The recombinant vector of claim 20, wherein the peptide linker is encoded by a polynucleotide sequence at least 75%, 80%, 85%, 90%, 95%, 96%, 97%, 98%, 99%, or 100% identical to the polynucleotide sequence of SEQ ID NO: 82.

23. The recombinant vector of any one of claims 1-22, wherein the fusion protein is membrane bound.

24. The recombinant vector of any one of claims 1-23, wherein the fusion protein comprises an amino acid sequence at least 90%, 95%, 96%, 97%, 98%, 99%, or 100% identical to the amino acid sequence of SEQ ID NO: 70 or 73.

25. The recombinant vector of any one of claims 1-22, wherein the fusion protein is encoded by a polynucleotide sequence at least 75%, 80%, 85%, 90%, 95%, 96%, 97%, 98%, 99%, or 100% identical to the polynucleotide sequence of SEQ ID NO: 71 or 74.

26. The recombinant vector of any one of claims 1-24, wherein the Cα region comprises an amino acid sequence at least 90%, 95%, 96%, 97%, 98%, 99%, or 100% identical to the amino acid sequence of SEQ ID NO: 40-49.

27. The recombinant vector of any one of claims 1-24, wherein the Cα region is encoded by a polynucleotide sequence at least 75%, 80%, 85%, 90%, 95%, 96%, 97%, 98%, 99%, or 100% identical to the polynucleotide sequence of SEQ ID NO: 55, 57, or 58.

28. The recombinant vector of any one of claims 1-26, wherein the Cβ region comprises an amino acid sequence at least 90%, 95%, 96%, 97%, 98%, 99%, or 100% identical to the amino acid sequence of SEQ ID NO: 50-54 or 60.

29. The recombinant vector of any one of claims 1-26, wherein the Cβ region is encoded by a polynucleotide sequence at least 75%, 80%, 85%, 90%, 95%, 96%, 97%, 98%, 99%, or 100% identical to the polynucleotide sequence of SEQ ID NO: 56 or 59.

30. The recombinant vector of any one of claims 1-29, wherein the polycistronic polynucleotide comprises, in order from 5′ to 3′: the first polynucleotide sequence, the second polynucleotide sequence, the third polynucleotide sequence, the fourth polynucleotide sequence, and the fifth polynucleotide sequence.

31. The recombinant vector of claim 30, wherein the first polynucleotide sequence and the second polynucleotide sequence together comprise a first combination polynucleotide sequence that encodes the amino acid sequence of SEQ ID NO: 160; the third polynucleotide sequence and the fourth polynucleotide sequence together comprise a second combination polynucleotide sequence that encodes the amino acid sequence of SEQ ID NO: 168; or the third polynucleotide sequence, the fourth polynucleotide sequence, and the fifth polynucleotide sequence together comprise a third combination polynucleotide sequence that encodes the amino acid sequence of SEQ ID NO: 161.

32. The recombinant vector of claim 31, wherein the first combination polynucleotide sequence comprises the polynucleotide sequence of SEQ ID NO: 230; the second combination polynucleotide sequence comprises the polynucleotide sequence of SEQ ID NO: 231; or the third combination polynucleotide sequence comprises the polynucleotide sequence of SEQ ID NO: 232.

33. The recombinant vector of claim 30, wherein the first polynucleotide sequence and the second polynucleotide sequence together comprise a first combination polynucleotide sequence that encodes the amino acid sequence of SEQ ID NO: 160; and the third polynucleotide sequence, the fourth polynucleotide sequence, and the fifth polynucleotide sequence together comprise a third combination polynucleotide sequence that encodes the amino acid sequence of SEQ ID NO: 161.

34. The recombinant vector of claim 30, wherein the first polynucleotide sequence and the second polynucleotide sequence together comprise a first combination polynucleotide sequence that encodes the amino acid sequence of SEQ ID NO: 180 or 210; and the third polynucleotide sequence, the fourth polynucleotide sequence, and the fifth polynucleotide sequence together comprise a third combination polynucleotide sequence that encodes the amino acid sequence of SEQ ID NO: 181.

35. The recombinant vector of claim 33, wherein the first combination polynucleotide sequence comprises the polynucleotide sequence of SEQ ID NO: 230; and the third combination polynucleotide sequence comprises the polynucleotide sequence of SEQ ID NO: 232.

36. The recombinant vector of claim 35, wherein the first combination polynucleotide sequence comprises the polynucleotide sequence of SEQ ID NO: 250 or 270; and the third combination polynucleotide sequence comprises the polynucleotide sequence of SEQ ID NO: 252.

37. The recombinant vector of any one of claims 1-29, wherein the polycistronic polynucleotide comprises, in order from 5′ to 3′: the first polynucleotide sequence, the fourth polynucleotide sequence, the third polynucleotide sequence, the second polynucleotide sequence, and the fifth polynucleotide sequence.

38. The recombinant vector of claim 37, wherein the first polynucleotide sequence and the fourth polynucleotide sequence together comprise a fourth combination polynucleotide sequence that encodes the amino acid sequence of SEQ ID NO: 162; the third polynucleotide sequence and the second polynucleotide sequence together comprise a fifth combination polynucleotide sequence that encodes the amino acid sequence of SEQ ID NO: 166; or the third polynucleotide sequence, the second polynucleotide sequence, and the fifth polynucleotide sequence together comprise a sixth combination polynucleotide sequence that encodes the amino acid sequence of SEQ ID NO: 163.

39. The recombinant vector of claim 38, wherein the fourth combination polynucleotide sequence comprises the polynucleotide sequence of SEQ ID NO: 233; the fifth combination polynucleotide sequence comprises the polynucleotide sequence of SEQ ID NO: 234; or the sixth combination polynucleotide sequence comprises the polynucleotide sequence of SEQ ID NO: 235.

40. The recombinant vector of claim 37, wherein the first polynucleotide sequence and the fourth polynucleotide sequence together comprise a fourth combination polynucleotide sequence that encodes the amino acid sequence of SEQ ID NO: 162; and the third polynucleotide sequence, the second polynucleotide sequence, and the fifth polynucleotide sequence together comprise a sixth combination polynucleotide sequence that encodes the amino acid sequence of SEQ ID NO: 163.

41. The recombinant vector of claim 37, wherein the first polynucleotide sequence and the fourth polynucleotide sequence together comprise a fourth combination polynucleotide sequence that encodes the amino acid sequence of SEQ ID NO: 182 or 212; and the third polynucleotide sequence, the second polynucleotide sequence, and the fifth polynucleotide sequence together comprise a sixth combination polynucleotide sequence that encodes the amino acid sequence of SEQ ID NO: 183.

42. The recombinant vector of claim 40, wherein the fourth combination polynucleotide sequence comprises the polynucleotide sequence of SEQ ID NO: 233; and the sixth combination polynucleotide sequence comprises the polynucleotide sequence of SEQ ID NO: 235.

43. The recombinant vector of claim 41, wherein the fourth combination polynucleotide sequence comprises the polynucleotide sequence of SEQ ID NO: 253 or 273; and the sixth combination polynucleotide sequence comprises the polynucleotide sequence of SEQ ID NO: 255.

44. The recombinant vector of any one of claims 1-29, wherein the polycistronic polynucleotide comprises, in order from 5′ to 3′: the first polynucleotide sequence, the second polynucleotide sequence, the fifth polynucleotide sequence, the fourth polynucleotide sequence, and the third polynucleotide sequence.

45. The recombinant vector of claim 44, wherein the first polynucleotide sequence and the second polynucleotide sequence together comprise a first combination polynucleotide sequence that encodes the amino acid sequence of SEQ ID NO: 160; the first polynucleotide sequence, the second polynucleotide sequence, and the fifth polynucleotide sequence together comprise a seventh combination polynucleotide sequence that encodes the amino acid sequence of SEQ ID NO: 169; the fifth polynucleotide sequence and the fourth polynucleotide sequence together comprise an eighth combination polynucleotide sequence that encodes the amino acid sequence of SEQ ID NO: 173; or the first polynucleotide sequence, the second polynucleotide sequence, the fifth polynucleotide sequence, and the fourth polynucleotide sequence together comprise a ninth combination polynucleotide sequence that encodes the amino acid sequence of SEQ ID NO: 164.

46. The recombinant vector of claim 45, wherein the first combination polynucleotide sequence comprises the polynucleotide sequence of SEQ ID NO: 230; the seventh combination polynucleotide sequence comprises the polynucleotide sequence of SEQ ID NO: 236; the eighth combination polynucleotide sequence comprises the polynucleotide sequence of SEQ ID NO: 237; or the ninth combination polynucleotide sequence comprises the polynucleotide sequence of SEQ ID NO: 238.

47. The recombinant vector of claim 44, wherein the first polynucleotide sequence, the second polynucleotide sequence, the fifth polynucleotide sequence, and the fourth polynucleotide sequence together comprise a ninth combination polynucleotide sequence that encodes the amino acid sequence of SEQ ID NO: 164; and the third polynucleotide sequence encodes the amino acid sequence of SEQ ID NO: 50.

48. The recombinant vector of claim 44, wherein the first polynucleotide sequence, the second polynucleotide sequence, the fifth polynucleotide sequence, and the fourth polynucleotide sequence together comprise a ninth combination polynucleotide sequence that encodes the amino acid sequence of SEQ ID NO: 184 or 214; and the third polynucleotide sequence encodes the amino acid sequence of SEQ ID NO: 51.

49. The recombinant vector of claim 47, wherein the ninth combination polynucleotide sequence comprises the polynucleotide sequence of SEQ ID NO: 238; and the third polynucleotide sequence comprises the polynucleotide sequence of SEQ ID NO: 59.

50. The recombinant vector of claim 48, wherein the ninth combination polynucleotide sequence comprises the polynucleotide sequence of SEQ ID NO: 258 or 278; and the third polynucleotide sequence comprises the polynucleotide sequence of SEQ ID NO: 56.

51. The recombinant vector of any one of claims 1-29, wherein the polycistronic polynucleotide comprises, in order from 5′ to 3′: the first polynucleotide sequence, the fourth polynucleotide sequence, the fifth polynucleotide sequence, the second polynucleotide sequence, and the third polynucleotide sequence.

52. The recombinant vector of claim 51, wherein the first polynucleotide sequence and the fourth polynucleotide sequence together comprise a fourth combination polynucleotide sequence that encodes the amino acid sequence of SEQ ID NO: 162; the first polynucleotide sequence, the fourth polynucleotide sequence, and the fifth polynucleotide sequence together comprise a tenth combination polynucleotide sequence that encodes the amino acid sequence of SEQ ID NO: 167; the fifth polynucleotide sequence and the second polynucleotide sequence together comprise an eleventh combination polynucleotide sequence that encodes the amino acid sequence of SEQ ID NO: 172; or the first polynucleotide sequence, the fourth polynucleotide sequence, the fifth polynucleotide sequence, and the second polynucleotide sequence together comprise a twelfth combination polynucleotide sequence that encodes the amino acid sequence of SEQ ID NO: 165.

53. The recombinant vector of claim 52, wherein the fourth combination polynucleotide sequence comprises the polynucleotide sequence of SEQ ID NO: 233; the tenth combination polynucleotide sequence comprises the polynucleotide sequence of SEQ ID NO: 239; the eleventh combination polynucleotide sequence comprises the polynucleotide sequence of SEQ ID NO: 240; or the twelfth combination polynucleotide sequence comprises the polynucleotide sequence of SEQ ID NO: 241.

54. The recombinant vector of claim 51, wherein the first polynucleotide sequence, the fourth polynucleotide sequence, the fifth polynucleotide sequence, and the second polynucleotide sequence together comprise a twelfth combination polynucleotide sequence that encodes the amino acid sequence of SEQ ID NO: 165; and the third polynucleotide sequence encodes the amino acid sequence of SEQ ID NO: 50.

55. The recombinant vector of claim 51, wherein the first polynucleotide sequence, the fourth polynucleotide sequence, the fifth polynucleotide sequence, and the second polynucleotide sequence together comprise a twelfth combination polynucleotide sequence that encodes the amino acid sequence of SEQ ID NO: 185 or 215; and the third polynucleotide sequence encodes the amino acid sequence of SEQ ID NO: 51.

56. The recombinant vector of claim 54, wherein the twelfth combination polynucleotide sequence comprises the polynucleotide sequence of SEQ ID NO: 241; and the third polynucleotide sequence comprises the polynucleotide sequence of SEQ ID NO: 59.

57. The recombinant vector of claim 55, wherein the twelfth combination polynucleotide sequence comprises the polynucleotide sequence of SEQ ID NO: 261 or 281; and the third polynucleotide sequence comprises the polynucleotide sequence of SEQ ID NO: 56.

58. The recombinant vector of any one of claims 1-29, wherein the polycistronic polynucleotide comprises, in order from 5′ to 3′: the third polynucleotide sequence, the second polynucleotide sequence, the first polynucleotide sequence, the fourth polynucleotide sequence, and the fifth polynucleotide sequence.

59. The recombinant vector of claim 58, wherein the first polynucleotide sequence and the fourth polynucleotide sequence together comprise a fourth combination polynucleotide sequence that encodes the amino acid sequence of SEQ ID NO: 162; the third polynucleotide sequence and the second polynucleotide sequence together comprise a fifth combination polynucleotide sequence that encodes the amino acid sequence of SEQ ID NO: 166; or the first polynucleotide sequence, the fourth polynucleotide sequence, and the fifth polynucleotide sequence together comprise a tenth combination polynucleotide sequence that encodes the amino acid sequence of SEQ ID NO: 167.

60. The recombinant vector of claim 59, wherein the fourth combination polynucleotide sequence comprises the polynucleotide sequence of SEQ ID NO: 233; the fifth combination polynucleotide sequence comprises the polynucleotide sequence of SEQ ID NO: 234; or the tenth combination polynucleotide sequence comprises the polynucleotide sequence of SEQ ID NO: 239.

61. The recombinant vector of claim 58, wherein the third polynucleotide sequence and the second polynucleotide sequence together comprise a fifth combination polynucleotide sequence that encodes the amino acid sequence of SEQ ID NO: 166; and the first polynucleotide sequence, the fourth polynucleotide sequence, and the fifth polynucleotide sequence together comprise a tenth combination polynucleotide sequence that encodes the amino acid sequence of SEQ ID NO: 167.

62. The recombinant vector of claim 58, wherein the third polynucleotide sequence and the second polynucleotide sequence together comprise a fifth combination polynucleotide sequence that encodes the amino acid sequence of SEQ ID NO: 186; and the first polynucleotide sequence, the fourth polynucleotide sequence, and the fifth polynucleotide sequence together comprise a tenth combination polynucleotide sequence that encodes the amino acid sequence of SEQ ID NO: 187 or 217.

63. The recombinant vector of claim 61, wherein the fifth combination polynucleotide sequence comprises the polynucleotide sequence of SEQ ID NO: 234; and the tenth combination polynucleotide sequence comprises the polynucleotide sequence of SEQ ID NO: 239.

64. The recombinant vector of claim 62, wherein the fifth combination polynucleotide sequence comprises the polynucleotide sequence of SEQ ID NO: 254; and the tenth combination polynucleotide sequence comprises the polynucleotide sequence of SEQ ID NO: 259 or 279.

65. The recombinant vector of any one of claims 1-29, wherein the polycistronic polynucleotide comprises, in order from 5′ to 3′: the third polynucleotide sequence, the fourth polynucleotide sequence, the first polynucleotide sequence, the second polynucleotide sequence, and the fifth polynucleotide sequence.

66. The recombinant vector of claim 65, wherein the first polynucleotide sequence and the second polynucleotide sequence together comprise a first combination polynucleotide sequence that encodes the amino acid sequence of SEQ ID NO: 160; the third polynucleotide sequence, and the fourth polynucleotide sequence together comprise a second combination polynucleotide sequence that encodes the amino acid sequence of SEQ ID NO: 168; or the first polynucleotide sequence, the second polynucleotide sequence, and the fifth polynucleotide sequence together comprise a seventh combination polynucleotide sequence that encodes the amino acid sequence of SEQ ID NO: 169.

67. The recombinant vector of claim 66, wherein the first combination polynucleotide sequence comprises the polynucleotide sequence of SEQ ID NO: 230; the second combination polynucleotide sequence comprises the polynucleotide sequence of SEQ ID NO: 231; or the seventh combination polynucleotide sequence comprises the polynucleotide sequence of SEQ ID NO: 236.

68. The recombinant vector of claim 65, wherein the third polynucleotide sequence, and the fourth polynucleotide sequence together comprise a second combination polynucleotide sequence that encodes the amino acid sequence of SEQ ID NO: 168; and the first polynucleotide sequence, the second polynucleotide sequence, and the fifth polynucleotide sequence together comprise a seventh combination polynucleotide sequence that encodes the amino acid sequence of SEQ ID NO: 169.

69. The recombinant vector of claim 65, wherein the third polynucleotide sequence, and the fourth polynucleotide sequence together comprise a second combination polynucleotide sequence that encodes the amino acid sequence of SEQ ID NO: 188; and the first polynucleotide sequence, the second polynucleotide sequence, and the fifth polynucleotide sequence together comprise a seventh combination polynucleotide sequence that encodes the amino acid sequence of SEQ ID NO: 189 or 219.

70. The recombinant vector of claim 68, wherein the second combination polynucleotide sequence comprises the polynucleotide sequence of SEQ ID NO: 231; and the seventh combination polynucleotide sequence comprises the polynucleotide sequence of SEQ ID NO: 236.

71. The recombinant vector of claim 69, wherein the second combination polynucleotide sequence comprises the polynucleotide sequence of SEQ ID NO: 251; and the seventh combination polynucleotide sequence comprises the polynucleotide sequence of SEQ ID NO: 256 or 276.

72. The recombinant vector of any one of claims 1-29, wherein the polycistronic polynucleotide comprises, in order from 5′ to 3′: the third polynucleotide sequence, the second polynucleotide sequence, the fifth polynucleotide sequence, the fourth polynucleotide sequence, and the first polynucleotide sequence.

73. The recombinant vector of claim 72, wherein the third polynucleotide sequence and the second polynucleotide sequence together comprise a fifth combination polynucleotide sequence that encodes the amino acid sequence of SEQ ID NO: 166; the third polynucleotide sequence, the second polynucleotide sequence, and the fifth polynucleotide sequence together comprise a sixth combination polynucleotide sequence that encodes the amino acid sequence of SEQ ID NO: 163; the fifth polynucleotide sequence and the fourth polynucleotide sequence together comprise an eighth combination polynucleotide sequence that encodes the amino acid sequence of SEQ ID NO: 173; or the third polynucleotide sequence, the second polynucleotide sequence, the fifth polynucleotide sequence, and the fourth polynucleotide sequence together comprise a thirteenth combination polynucleotide sequence that encodes the amino acid sequence of SEQ ID NO: 170.

74. The recombinant vector of claim 73, wherein the fifth combination polynucleotide sequence comprises the polynucleotide sequence of SEQ ID NO: 234; the sixth combination polynucleotide sequence comprises the polynucleotide sequence of SEQ ID NO: 235; the eighth combination polynucleotide sequence comprises the polynucleotide sequence of SEQ ID NO: 237; or the thirteenth combination polynucleotide sequence comprises the polynucleotide sequence of SEQ ID NO: 242.

75. The recombinant vector of claim 72, wherein the third polynucleotide sequence, the second polynucleotide sequence, the fifth polynucleotide sequence, and the fourth polynucleotide sequence together comprise a thirteenth combination polynucleotide sequence that encodes the amino acid sequence of SEQ ID NO: 170; and the first polynucleotide sequence encodes the amino acid sequence of SEQ ID NO: 40.

76. The recombinant vector of claim 72, wherein the third polynucleotide sequence, the second polynucleotide sequence, the fifth polynucleotide sequence, and the fourth polynucleotide sequence together comprise a thirteenth combination polynucleotide sequence that encodes the amino acid sequence of SEQ ID NO: 190; and the first polynucleotide sequence encodes the amino acid sequence of SEQ ID NO: 41 or 42.

77. The recombinant vector of claim 75, wherein the thirteenth combination polynucleotide sequence comprises the polynucleotide sequence of SEQ ID NO: 242; and the first polynucleotide sequence comprises the polynucleotide sequence of SEQ ID NO: 57.

78. The recombinant vector of claim 76, wherein the thirteenth combination polynucleotide sequence comprises the polynucleotide sequence of SEQ ID NO: 262; and the first polynucleotide sequence comprises the polynucleotide sequence of SEQ ID NO: 55 or 58.

79. The recombinant vector of any one of claims 1-29, wherein the polycistronic polynucleotide comprises, in order from 5′ to 3′: the third polynucleotide sequence, the fourth polynucleotide sequence, the fifth polynucleotide sequence, the second polynucleotide sequence, and the first polynucleotide sequence.

80. The recombinant vector of claim 79, wherein the third polynucleotide sequence and the fourth polynucleotide sequence together comprise a second combination polynucleotide sequence that encodes the amino acid sequence of SEQ ID NO: 168; the third polynucleotide sequence, the fourth polynucleotide sequence, and the fifth polynucleotide sequence together comprise a third combination polynucleotide sequence that encodes the amino acid sequence of SEQ ID NO: 161; the fifth polynucleotide sequence and the second polynucleotide sequence together comprise an eleventh combination polynucleotide sequence that encodes the amino acid sequence of SEQ ID NO: 172; or the third polynucleotide sequence, the fourth polynucleotide sequence, the fifth polynucleotide sequence, and the second polynucleotide sequence together comprise a fourteenth combination polynucleotide sequence that encodes the amino acid sequence of SEQ ID NO: 171.

81. The recombinant vector of claim 80, wherein the second combination polynucleotide sequence comprises the polynucleotide sequence of SEQ ID NO: 231; the third combination polynucleotide sequence comprises the polynucleotide sequence of SEQ ID NO: 232; the eleventh combination polynucleotide sequence comprises the polynucleotide sequence of SEQ ID NO: 240; or the fourteenth combination polynucleotide sequence comprises the polynucleotide sequence of SEQ ID NO: 243.

82. The recombinant vector of claim 79, wherein the third polynucleotide sequence, the fourth polynucleotide sequence, the fifth polynucleotide sequence, and the second polynucleotide sequence together comprise a fourteenth combination polynucleotide sequence that encodes the amino acid sequence of SEQ ID NO: 171; and the first polynucleotide sequence encodes the amino acid sequence of SEQ ID NO: 40.

83. The recombinant vector of claim 79, wherein the third polynucleotide sequence, the fourth polynucleotide sequence, the fifth polynucleotide sequence, and the second polynucleotide sequence together comprise a fourteenth combination polynucleotide sequence that encodes the amino acid sequence of SEQ ID NO: 191; and the first polynucleotide sequence encodes the amino acid sequence of SEQ ID NO: 41 or 42.

84. The recombinant vector of claim 82, wherein the fourteenth combination polynucleotide sequence comprises the polynucleotide sequence of SEQ ID NO: 243; and the first polynucleotide sequence comprises the polynucleotide sequence of SEQ ID NO: 57.

85. The recombinant vector of claim 83, wherein the fourteenth combination polynucleotide sequence comprises the polynucleotide sequence of SEQ ID NO: 263; and the first polynucleotide sequence comprises the polynucleotide sequence of SEQ ID NO: 55 or 58.

86. The recombinant vector of any one of claims 1-29, wherein the polycistronic polynucleotide comprises, in order from 5′ to 3′: the fifth polynucleotide sequence, the second polynucleotide sequence, the first polynucleotide sequence, the fourth polynucleotide sequence, and the third polynucleotide sequence.

87. The recombinant vector of claim 86, wherein the first polynucleotide sequence and the fourth polynucleotide sequence together comprise a fourth combination polynucleotide sequence that encodes the amino acid sequence of SEQ ID NO: 162; or the fifth polynucleotide sequence and the second polynucleotide sequence together comprise an eleventh combination polynucleotide sequence that encodes the amino acid sequence of SEQ ID NO: 172.

88. The recombinant vector of claim 87, wherein the fourth combination polynucleotide sequence comprises the polynucleotide sequence of SEQ ID NO: 233; or the eleventh combination polynucleotide sequence comprises the polynucleotide sequence of SEQ ID NO: 240.

89. The recombinant vector of claim 86, wherein the first polynucleotide sequence and the fourth polynucleotide sequence together comprise a fourth combination polynucleotide sequence that encodes the amino acid sequence of SEQ ID NO: 162; the fifth polynucleotide sequence and the second polynucleotide sequence together comprise an eleventh combination polynucleotide sequence that encodes the amino acid sequence of SEQ ID NO: 172; and the third polynucleotide sequence encodes the amino acid sequence of SEQ ID NO: 50.

90. The recombinant vector of claim 86, wherein the first polynucleotide sequence and the fourth polynucleotide sequence together comprise a fourth combination polynucleotide sequence that encodes the amino acid sequence of SEQ ID NO: 182 or 212; the fifth polynucleotide sequence and the second polynucleotide sequence together comprise an eleventh combination polynucleotide sequence that encodes the amino acid sequence of SEQ ID NO: 172; and the third polynucleotide sequence encodes the amino acid sequence of SEQ ID NO: 51.

91. The recombinant vector of claim 89, wherein the fourth combination polynucleotide sequence comprises the polynucleotide sequence of SEQ ID NO: 233; and the eleventh combination polynucleotide sequence comprises the polynucleotide sequence of SEQ ID NO: 240; and the third polynucleotide sequence comprises the polynucleotide sequence of SEQ ID NO: 59.

92. The recombinant vector of claim 90, wherein the fourth combination polynucleotide sequence comprises the polynucleotide sequence of SEQ ID NO: 253 or 273; the eleventh combination polynucleotide sequence comprises the polynucleotide sequence of SEQ ID NO: 240; and the third polynucleotide sequence comprises the polynucleotide sequence of SEQ ID NO: 56.

93. The recombinant vector of any one of claims 1-29, wherein the polycistronic polynucleotide comprises, in order from 5′ to 3′: the fifth polynucleotide sequence, the fourth polynucleotide sequence, the first polynucleotide sequence, the second polynucleotide sequence, and the third polynucleotide sequence.

94. The recombinant vector of claim 93, wherein the first polynucleotide sequence and the second polynucleotide sequence together comprise a first combination polynucleotide sequence that encodes the amino acid sequence of SEQ ID NO: 160; or the fifth polynucleotide sequence and the fourth polynucleotide sequence together comprise an eighth combination polynucleotide sequence that encodes the amino acid sequence of SEQ ID NO: 173.

95. The recombinant vector of claim 94, wherein the first combination polynucleotide sequence comprises the polynucleotide sequence of SEQ ID NO: 230; or the eighth combination polynucleotide sequence comprises the polynucleotide sequence of SEQ ID NO: 237.

96. The recombinant vector of claim 93, wherein the first polynucleotide sequence and the second polynucleotide sequence together comprise a first combination polynucleotide sequence that encodes the amino acid sequence of SEQ ID NO: 160; the fifth polynucleotide sequence and the fourth polynucleotide sequence together comprise an eighth combination polynucleotide sequence that encodes the amino acid sequence of SEQ ID NO: 173; and the third polynucleotide sequence encodes the amino acid sequence of SEQ ID NO: 50.

97. The recombinant vector of claim 93, wherein the first polynucleotide sequence and the second polynucleotide sequence together comprise a first combination polynucleotide sequence that encodes the amino acid sequence of SEQ ID NO: 180 or 210; the fifth polynucleotide sequence and the fourth polynucleotide sequence together comprise an eighth combination polynucleotide sequence that encodes the amino acid sequence of SEQ ID NO: 173; and the third polynucleotide sequence encodes the amino acid sequence of SEQ ID NO: 51.

98. The recombinant vector of claim 96, wherein the first combination polynucleotide sequence comprises the polynucleotide sequence of SEQ ID NO: 230; the eighth combination polynucleotide sequence comprises the polynucleotide sequence of SEQ ID NO: 237; and the third polynucleotide sequence comprises the polynucleotide sequence of SEQ ID NO: 59.

99. The recombinant vector of claim 97, wherein the first combination polynucleotide sequence comprises the polynucleotide sequence of SEQ ID NO: 250 or 270; the eighth combination polynucleotide sequence comprises the polynucleotide sequence of SEQ ID NO: 237; and the third polynucleotide sequence comprises the polynucleotide sequence of SEQ ID NO: 56.

100. The recombinant vector of any one of claims 1-29, wherein the polycistronic polynucleotide comprises, in order from 5′ to 3′: the fifth polynucleotide sequence, the second polynucleotide sequence, the third polynucleotide sequence, the fourth polynucleotide sequence, and the first polynucleotide sequence.

101. The recombinant vector of claim 100, wherein the third polynucleotide sequence and the fourth polynucleotide sequence together comprise a second combination polynucleotide sequence that encodes the amino acid sequence of SEQ ID NO: 168; or the fifth polynucleotide sequence and the second polynucleotide sequence together comprise an eleventh combination polynucleotide sequence that encodes the amino acid sequence of SEQ ID NO: 172.

102. The recombinant vector of claim 101, wherein the second combination polynucleotide sequence comprises the polynucleotide sequence of SEQ ID NO: 231; or the eleventh combination polynucleotide sequence comprises the polynucleotide sequence of SEQ ID NO: 240.

103. The recombinant vector of claim 100, wherein the third polynucleotide sequence and the fourth polynucleotide sequence together comprise a second combination polynucleotide sequence that encodes the amino acid sequence of SEQ ID NO: 168; the fifth polynucleotide sequence and the second polynucleotide sequence together comprise an eleventh combination polynucleotide sequence that encodes the amino acid sequence of SEQ ID NO: 172; and the first polynucleotide sequence encodes the amino acid sequence of SEQ ID NO: 40.

104. The recombinant vector of claim 100, wherein the third polynucleotide sequence and the fourth polynucleotide sequence together comprise a second combination polynucleotide sequence that encodes the amino acid sequence of SEQ ID NO: 188; the fifth polynucleotide sequence and the second polynucleotide sequence together comprise an eleventh combination polynucleotide sequence that encodes the amino acid sequence of SEQ ID NO: 172; and the first polynucleotide sequence encodes the amino acid sequence of SEQ ID NO: 41 or 42.

105. The recombinant vector of claim 103, wherein the second combination polynucleotide sequence comprises the polynucleotide sequence of SEQ ID NO: 231; the eleventh combination polynucleotide sequence comprises the polynucleotide sequence of SEQ ID NO: 240; and the first polynucleotide sequence comprises the polynucleotide sequence of SEQ ID NO: 57.

106. The recombinant vector of claim 104, wherein the second combination polynucleotide sequence comprises the polynucleotide sequence of SEQ ID NO: 251; the eleventh combination polynucleotide sequence comprises the polynucleotide sequence of SEQ ID NO: 240; and the first polynucleotide sequence comprises the polynucleotide sequence of SEQ ID NO: 55 or 58.

107. The recombinant vector of any one of claims 1-29, wherein the polycistronic polynucleotide comprises, in order from 5′ to 3′: the fifth polynucleotide sequence, the fourth polynucleotide sequence, the third polynucleotide sequence, the second polynucleotide sequence, and the first polynucleotide sequence.

108. The recombinant vector of claim 107, wherein the third polynucleotide sequence and the second polynucleotide sequence together comprise a fifth combination polynucleotide sequence that encodes the amino acid sequence of SEQ ID NO: 166; or the fifth polynucleotide sequence and the fourth polynucleotide sequence together comprise an eighth combination polynucleotide sequence that encodes the amino acid sequence of SEQ ID NO: 173.

109. The recombinant vector of claim 108, wherein the fifth combination polynucleotide sequence comprises the polynucleotide sequence of SEQ ID NO: 234; or the eighth combination polynucleotide sequence comprises the polynucleotide sequence of SEQ ID NO: 237.

110. The recombinant vector of claim 107, wherein the third polynucleotide sequence and the second polynucleotide sequence together comprise a fifth combination polynucleotide sequence that encodes the amino acid sequence of SEQ ID NO: 166; the fifth polynucleotide sequence and the fourth polynucleotide sequence together comprise an eighth combination polynucleotide sequence that encodes the amino acid sequence of SEQ ID NO: 173; and the first polynucleotide sequence encodes the amino acid sequence of SEQ ID NO: 40.

111. The recombinant vector of claim 107, wherein the third polynucleotide sequence and the second polynucleotide sequence together comprise a fifth combination polynucleotide sequence that encodes the amino acid sequence of SEQ ID NO: 186; the fifth polynucleotide sequence and the fourth polynucleotide sequence together comprise an eighth combination polynucleotide sequence that encodes the amino acid sequence of SEQ ID NO: 173; and the first polynucleotide sequence encodes the amino acid sequence of SEQ ID NO: 41 or 42.

112. The recombinant vector of claim 110, wherein the fifth combination polynucleotide sequence comprises the polynucleotide sequence of SEQ ID NO: 234; the eighth combination polynucleotide sequence comprises the polynucleotide sequence of SEQ ID NO: 237; and the first polynucleotide sequence comprises the polynucleotide sequence of SEQ ID NO: 57.

113. The recombinant vector of claim 111, wherein the fifth combination polynucleotide sequence comprises the polynucleotide sequence of SEQ ID NO: 254; the eighth combination polynucleotide sequence comprises the polynucleotide sequence of SEQ ID NO: 237; and the first polynucleotide sequence comprises the polynucleotide sequence of SEQ ID NO: 55 or 58.

114. The recombinant vector of any one of claims 1-113, wherein the polycistronic polynucleotide further comprises
f. a sixth polynucleotide sequence that comprises a third 2A element; and
g. a seventh polynucleotide sequence that comprises a marker protein.

115. The recombinant vector of claim 114, wherein the third 2A element is a P2A element, a T2A element, an F2A element, or an E2A element.

116. The recombinant vector of any one of claims 114 or 115, wherein the marker protein comprises: domain III of HER1, or a functional fragment or functional variant thereof; an N-terminal portion of domain IV of HER1; and a transmembrane domain of CD28, or a functional fragment or functional variant thereof.

117. The recombinant vector of claim 116, wherein the domain III of HER1, or a functional fragment or functional variant thereof, comprises an amino acid sequence at least 90%, 95%, 96%, 97%, 98%, 99%, or 100% identical to the amino acid sequence of SEQ ID NO: 104.

118. The recombinant vector of claim 116, wherein the N-terminal portion of domain IV of HER1 comprises amino acids 1-40, 1-39, 1-38, 1-37, 1-36, 1-35, 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, or 1-10 of SEQ ID NO: 105.

119. The recombinant vector of claim 116, wherein the N-terminal portion of domain IV of HER1 comprises amino acids 1-21 of SEQ ID NO: 105.

120. The recombinant vector of claim 116, wherein the N-terminal portion of domain IV of HER1 comprises the amino acid sequence of SEQ ID NO: 106, or the amino acid sequence of SEQ ID NO: 106, comprising 1, 2, or 3 amino acid modifications.

121. The recombinant vector of any one of claims 114-120, wherein the transmembrane region of CD28 comprises the amino acid sequence of SEQ ID NO: 107, or the amino acid sequence of SEQ ID NO: 107, comprising 1, 2, or 3 amino acid modifications.

122. The recombinant vector of any one of claims 114-121, wherein the marker protein comprises an amino acid sequence at least 90%, 95%, 96%, 97%, 98%, 99%, or 100% identical to the amino acid sequence of SEQ ID NO: 100, 103, or 112.

123. The recombinant vector of any one of claims 1-122, wherein the Vα region comprises complementarity determining region 1α (CDR1α), CDR2α, and CDR3α, comprising the amino acid sequences of SEQ ID NO: 1001+10n, 1002+10n, and 1003+10n, respectively, wherein n is an integer from 0 to 79.

124. The recombinant vector of any one of claims 1-123, wherein the Vβ region comprises CDR1β, CDR2β, and CDR3β, comprising the amino acid sequences of SEQ ID NO: 2001+10n, 2002+10n, and 2003+10n, respectively, wherein n is an integer from 0 to 79.

125. The recombinant vector of any one of claims 1-124, wherein the Vα region comprises the CDR1a, CDR2α, and CDR3α from a Vα region comprising the amino acid sequence of SEQ ID NO: 1004+10n, 1005+10n, 1006+10n, or 1007+10n, wherein n is an integer from 0 to 79.

126. The recombinant vector of any one of claims 1-125, wherein the Vβ region comprises the CDR1β, CDR2β, and CDR30 from a Vβ region comprising the amino acid sequence of SEQ ID NO: 2004+10n, 2005+10n, 2006+10n, or 2007+10n, wherein n is an integer from 0 to 79.

127. The recombinant vector of any one of claims 1-126, wherein the Vα region comprises an amino acid sequence at least 90%, 95%, 96%, 97%, 98%, 99%, or 100% identical to the amino acid sequence of SEQ ID NO: 1004+10n, 1005+10n, 1006+10n, or 1007+10n, wherein n is an integer from 0 to 79.

128. The recombinant vector of any one of claims 1-127, wherein the Vβ region comprises an amino acid sequence at least 90%, 95%, 96%, 97%, 98%, 99%, or 100% identical to the amino acid sequence of SEQ ID NO: 2004+10n, 2005+10n, 2006+10n, or 2007+10n, wherein n is an integer from 0 to 79.

129. The recombinant vector of any one of claims 1-128, wherein the Vα region comprises an amino acid sequence at least 90%, 95%, 96%, 97%, 98%, 99%, or 100% identical to the amino acid sequence of SEQ ID NO: 1004+10n, wherein the Vβ region comprises an amino acid sequence at least 90%, 95%, 96%, 97%, 98%, 99%, or 100% identical to the amino acid sequence of SEQ ID NO: 2004+10n, and wherein n is an integer from 0 to 79; wherein the Vα region comprises an amino acid sequence at least 90%, 95%, 96%, 97%, 98%, 99%, or 100% identical to the amino acid sequence of SEQ ID NO: 1005+10n, wherein the Vβ region comprises an amino acid sequence at least 90%, 95%, 96%, 97%, 98%, 99%, or 100% identical to the amino acid sequence of SEQ ID NO: 2005+10n, and wherein n is an integer from 0 to 79; wherein the Vα region comprises an amino acid sequence at least 90%, 95%, 96%, 97%, 98%, 99%, or 100% identical to the amino acid sequence of SEQ ID NO: 1006+10n, wherein the Vβ region comprises an amino acid sequence at least 90%, 95%, 96%, 97%, 98%, 99%, or 100% identical to the amino acid sequence of SEQ ID NO: 2006+10n, and wherein n is an integer from 0 to 79; or wherein the Vα region comprises an amino acid sequence at least 90%, 95%, 96%, 97%, 98%, 99%, or 100% identical to the amino acid sequence of SEQ ID NO: 1007+10n, wherein the Vβ region comprises an amino acid sequence at least 90%, 95%, 96%, 97%, 98%, 99%, or 100% identical to the amino acid sequence of SEQ ID NO: 2007+10n, and wherein n is an integer from 0 to 79.

130. The recombinant vector of any one of claims 1-129, wherein the TCR alpha chain comprises an amino acid sequence at least 90%, 95%, 96%, 97%, 98%, 99%, or 100% identical to the amino acid sequence of SEQ ID NO: 1008+10n, wherein n is an integer from 0 to 79.

131. The recombinant vector of any one of claims 1-129, wherein the TCR alpha chain comprises an amino acid sequence at least 90%, 95%, 96%, 97%, 98%, 99%, or 100% identical to the amino acid sequence of SEQ ID NO: 1009+10n, wherein n is an integer from 0 to 79.

132. The recombinant vector of any one of claims 1-129, wherein the TCR alpha chain comprises an amino acid sequence at least 90%, 95%, 96%, 97%, 98%, 99%, or 100% identical to the amino acid sequence of SEQ ID NO: 1010+10n, wherein n is an integer from 0 to 79.

133. The recombinant vector of any one of claims 1-132, wherein the TCR beta chain comprises an amino acid sequence at least 90%, 95%, 96%, 97%, 98%, 99%, or 100% identical to the amino acid sequence of SEQ ID NO: 2008+10n, wherein n is an integer from 0 to 79.

134. The recombinant vector of any one of claims 1-132, wherein the TCR beta chain comprises an amino acid sequence at least 90%, 95%, 96%, 97%, 98%, 99%, or 100% identical to the amino acid sequence of SEQ ID NO: 2009+10n, wherein n is an integer from 0 to 79.

135. The recombinant vector of any one of claims 1-132, wherein the TCR beta chain comprises an amino acid sequence at least 90%, 95%, 96%, 97%, 98%, 99%, or 100% identical to the amino acid sequence of SEQ ID NO: 2010+10n, wherein n is an integer from 0 to 79.

136. The recombinant vector of any one of claims 1-135, wherein the transcriptional regulatory element comprises a promoter.

137. The recombinant vector of claim 136, wherein the promoter is a human elongation factor 1-alpha (hEF-1α) hybrid promoter.

138. The recombinant vector of claim 136 or 137, wherein the promoter comprises a polynucleotide sequence at least 75%, 80%, 85%, 90%, 95%, 96%, 97%, 98%, 99%, or 100% identical to the polynucleotide sequence of SEQ ID NO:150.

139. The recombinant vector of any one of claims 1-138, further comprising a polyA sequence at the 3′ end of the polycistronic expression cassette.

140. The recombinant vector of claim 139, wherein the polyA sequence comprises a polynucleotide sequence at least 75%, 80%, 85%, 90%, 95%, 96%, 97%, 98%, 99%, or 100% identical to the polynucleotide sequence of SEQ ID NO:151.

141. The recombinant vector of any one of claims 1-140, further comprising a Left inverted terminal repeat (ITR) and a Right ITR, wherein the Left ITR and the Right ITR flank the polycistronic expression cassette.

142. The recombinant vector of claim 141, which comprises, in order from 5′ to 3′:
i. the Left ITR;
ii. the transcriptional regulatory element;
iii. the first polynucleotide sequence;
iv. the second polynucleotide sequence;
v. the third polynucleotide sequence;
vi. the fourth polynucleotide sequence;
vii. the fifth polynucleotide sequence; and
viii. the Right ITR.

143. The recombinant vector of any one of claims 1-142, wherein the recombinant vector is a non-viral vector.

144. The recombinant vector of claim 143, wherein the non-viral vector is a plasmid.

145. The recombinant vector of any one of claims 1-142, wherein the recombinant vector is a viral vector.

146. The recombinant vector of any one of claims 1-145, wherein the recombinant vector is a polynucleotide.

147. A polynucleotide encoding an amino acid sequence at least 90%, 95%, 96%, 97%, 98%, 99%, or 100% identical to an amino acid sequence selected from the group consisting of SEQ ID NOs: 161, 163, 164, 165, 167, 169, 170, and 171.

148. A polynucleotide comprising a polynucleotide sequence at least 75%, 80%, 85%, 90%, 95%, 96%, 97%, 98%, 99%, or 100% identical to a polynucleotide sequence selected from the group consisting of SEQ ID NOs: 232, 235, 236, 238, 239, 241, 242, and 243.

149. A population of cells that comprise the recombinant vector of any one of claims 1-146 or the polynucleotide of claim 147 or 148.

150. The population of cells of claim 149, wherein the recombinant vector or the polynucleotide is integrated into the genome of the population of cells.

151. The population of cells of claims 149 or 150, wherein the cells are immune effector cells.

152. The population of cells of claim 151, wherein the immune effector cells are selected from the group consisting of T cells, natural killer (NK) cells, B cells, mast cells, and myeloid-derived phagocytes.

153. The population of cells of claim 152, wherein the immune effector cells are one or more T cells selected from the group consisting of naïve T cells (CD4+ or CD8+); killer CD8+ T cells; cytotoxic CD4+ T cells; helper CD4+ T cells; CD4+ T cells corresponding to Th1, Th2, Th9, Th17, Th22, follicular helper (Th), regulatory (Treg) lineages; tumor infiltrating lymphocytes (TILs); and memory T cells (central memory, effector memory, stem cell memory, stem cell-like memory).

154. The population of cells of claim 152, comprising alpha/beta T cells, gamma/delta T cells, or natural killer T (NKT) cells.

155. The population of cells of claim 152, comprising CD4⁺ T cells, CD8⁺ T cells, or both CD4⁺ T cells and CD8⁺ T cells.

156. The population of cells of any one of claims 149-155, wherein the cells are ex vivo.

157. The population of cells of any one of claims 149-156, wherein the cells are human.

158. The population of cells of any one of claims 149-157, wherein the population of cells are T cells that comprise more than 5%, 10%, 15%, 20%, 25%, 30%, 35%, 40%, 45% or 50% CD45RA+CD45RO−CD62L+CD95+ cells.

159. The population of cells of any one of claims 149-157, wherein the population of cells are T cells that comprise more than 5%, 10%, 15%, 20%, 25%, 30%, 35%, 40%, 45% or 50% CD45RA+CD45RO+CD62L+CD95+ cells.

160. A population of cells comprising a polycistronic expression cassette comprising
a. a first cistron comprising a polynucleotide sequence that encodes a fusion protein that comprises IL-15, or a functional fragment or functional variant thereof, and IL-15Rα, or a functional fragment or functional variant thereof,
b. a second cistron comprising a polynucleotide sequence that encodes a TCR beta chain comprising a Vβ region and a Cβ region; and
c. a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO−CD62L+CD95+ cells.

161. A population of cells comprising a polycistronic expression cassette comprising
a. a first cistron comprising a polynucleotide sequence that encodes a fusion protein that comprises IL-15, or a functional fragment or functional variant thereof, and IL-15Rα, or a functional fragment or functional variant thereof,
b. a second cistron comprising a polynucleotide sequence that encodes a TCR beta chain comprising a Vβ region and a Cβ region; and
c. a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO+CD62L+CD95+ cells.

162. A method of producing a population of engineered cells, comprising:
introducing into a population of cells the recombinant vector of claim 141 or 142, and a DNA transposase or a polynucleotide encoding a DNA transposase; and
culturing the population of cells under conditions wherein the transposase integrates the polycistronic expression cassette into the genome of the population of cells, thereby producing the population of engineered cells.

163. The method of claim 162, wherein the Left ITR and the Right ITR are ITRs of a DNA transposon selected from the group consisting of a Sleeping Beauty transposon, a piggyBac transposon, a TcBuster transposon, and a Tol2 transposon.

164. The method of claim 163, wherein the DNA transposon is the Sleeping Beauty transposon.

165. The method of any one of claims 162-164, wherein the transposase is a Sleeping Beauty transposase.

166. The method of claim 165, wherein the Sleeping Beauty transposase is selected from the group consisting of SB11, SB10, SB100X, hSB110, and hSB81.

167. The method of claim 166, wherein the Sleeping Beauty transposase is SB11.

168. The method of claim 167, wherein the SB11 comprises an amino acid sequence at least 90%, 95%, 96%, 97%, 98%, 99%, or 100% identical to the amino acid sequence of SEQ ID NO: 300.

169. The method of claim 167, wherein the SB11 is encoded by a polynucleotide sequence at least 75%, 80%, 85%, 90%, 95%, 96%, 97%, 98%, 99%, or 100% identical to the polynucleotide sequence of SEQ ID NO: 301.

170. The method of any one of claims 162-169, wherein the polynucleotide encoding the DNA transposase is a DNA vector or an RNA vector.

171. The method of any one of claims 162-170, wherein the Left ITR comprises a polynucleotide sequence at least 75%, 80%, 85%, 90%, 95%, 96%, 97%, 98%, 99%, or 100% identical to the polynucleotide sequence of SEQ ID NO: 290 or 291; and the Right ITR comprises a polynucleotide sequence at least 75%, 80%, 85%, 90%, 95%, 96%, 97%, 98%, 99%, or 100% identical to the polynucleotide sequence of SEQ ID NO: 292, 293 or 294.

172. The method of any one of claims 162-171, wherein the recombinant vector, and the DNA transposase or polynucleotide encoding the DNA transposase, are introduced to the population of cells using electroporation, sonication, calcium phosphate precipitation, lipofection, particle bombardment, microinjection, mechanical deformation by passage through a microfluidic device, or a colloidal dispersion system.

173. The method of claim 172, wherein the recombinant vector, and the DNA transposase or polynucleotide encoding the DNA transposase, are introduced to the population of cells using electroporation.

174. The method of any one of claims 162-173, wherein the method is completed within 30 days, 25 days, 20 days, 15 days, 14 days, 10 days, 7 days, 6 days, 5 days, 4 days, 3 days, 2 days, or 1 day.

175. The method of any one of claims 162-173, wherein the method is completed in less than 30 days, 25 days, 20 days, 15 days, 14 days, 10 days, 7 days, 6 days, 5 days, 4 days, 3 days, 2 days, or 1 day.

176. The method of any one of claims 162-175, wherein the population of cells is cryopreserved and thawed before introduction of the recombinant vector and the DNA transposase or polynucleotide encoding the DNA transposase.

177. The method of any one of claims 162-176, wherein the population of cells is rested before introduction of the recombinant vector and the DNA transposase or polynucleotide encoding the DNA transposase.

178. The method of any one of claims 162-177, wherein the population of cells is not rested before introduction of the recombinant vector and the DNA transposase or polynucleotide encoding the DNA transposase.

179. The method of any one of claims 162-178, wherein the population of cells comprises expanded human ex vivo cells.

180. The method of any one of claims 162-179, wherein the population of cells is not activated ex vivo.

181. The method of any one of claims 162-180, wherein the population of cells comprises T cells.

182. A method of treating cancer in a subject in need thereof comprising administering to the subject a therapeutically effective amount of the population of cells of any one of claims 149-161, thereby treating the cancer.

183. The method of claim 182, wherein the cancer is selected from lung, cholangiocarcinoma, pancreatic, colorectal, gynecological and ovarian cancer.

184. A method of treating an autoimmune disease or disorder in a subject in need thereof comprising administering to the subject a therapeutically effective amount of the population of cells of any one of claims 149-161, thereby treating the autoimmune disease or disorder.
